(19) United States
(12) Patent Application Publication
(10) Pub. No.: US 2025/0289840 A1
(43) Pub. Date: Sep. 18, 2025

(54) RNA STABILIZING SUBSTANCES AND METHODS OF USE

(71) Applicant: Team Medical, LLC, Boulder, CO (US)

(72) Inventors: Kyle HEIM, Boulder, CO (US); Warren P. HEIM, Boulder, CO (US)

(21) Appl. No.: 19/059,030

(22) Filed: Feb. 20, 2025

Related U.S. Application Data
(60) Provisional application No. 63/555,500, filed on Feb. 20, 2024.
(60) Provisional application No. 63/675,023, filed on Jul. 24, 2024.

(51) Int. Cl. C07H 1/06; A61K 31/7105; A61K 39/00; A61K 47/26; A61K 47/30

(52) U.S. Cl. CPC C07H 1/06; A61K 31/7105; A61K 39/00; A61K 47/26; A61K 47/30; A61K 2039/53

(57) ABSTRACT

The present disclosure provides compositions and methods for producing and manufacturing compositions comprising at least one or more RNA stabilizing substance and at least one or more substance comprising extracellular RNA, wherein the compositions and methods of use provided herein improve the storage and stability of substances comprising extracellular RNA or based on RNA at temperatures above freezing temperatures without lyophilization. The present disclosure, further provides compositions for improving the stability of pharmaceutical compositions comprising RNA substances or based on RNA substances, including the stability of RNA in conjunction with nanoparticles or lipid-nanoparticles and applications for improving the storage and stability of pharmaceutical compositions comprising RNA or RNA substances above freezing temperatures without lyophilization. The present disclosure also provides descriptions of kits and methods for providing or producing kits for producing compositions comprising at least one or more RNA stabilizing substance.

### FIELD OF THE INVENTION

The present disclosure relates to compositions and methods of using the compositions to improve the stability of various types of extracellular RNA and substances based on various types of extracellular RNA for storage and use in non-clinical applications and clinical applications including for therapeutic uses to diagnose the health or improve the health of living organisms including plants and animals including treating humans including diagnosis of diseases and treatment of diseases or other adverse health effects in animals including in humans.

### BACKGROUND OF THE INVENTION

Ribonucleic acid (RNA) is responsible for the transcription of the genetic information stored in deoxyribonucleic acid (DNA) in a form that can be used in cells to synthesize proteins. The use of therapeutic RNA to beneficially produce proteins, regulate gene expression, or induce immune responses to specific antigens and biomarkers has become an emerging part of the field of nucleic acid therapy. The potential applications and recognized advantages of RNA based nucleic acid therapies continue to increase. For example, the recent COVID-19 infections in humans have led to vaccines developed using messenger RNA (mRNA). RNA therapies, such as vaccines using mRNA have advantages compared to other therapies, such as traditional vaccines that use inactivated, attenuated, or genetically modified microorganisms, purified antigens, or viral vectors. These other therapies can lead to adverse reactions, side effects, allergic reactions, or can develop mutations, either during manufacturing or administration, that can alter the efficacy or lead to safety concerns. RNA encodes the genetic information for the target therapy to be produced endogenously within the host cell without the need to synthesize and purify individual antigens, thereby creating greater flexibility to specifically tailor different therapies for a variety of diseases and simplifying the manufacturing process by allowing the target cells to facilitate the production of the necessary proteins and reduce or eliminate the complications of traditional vaccines.

Using RNA for nucleic acid therapies has distinct advantages compared to DNA based therapies due to the relatively short half-life of RNA and the transient message encoded within the RNA that does not require entry into the nucleus for proper expression necessary to carry out the desired function. Furthermore, DNA can potentially integrate into the host cell genome and alter genomic DNA or also become inherited by progeny. However, the investigation of uses of RNA and the production of products using RNA is complicated by the limited stability of RNA. RNA is not as stable as DNA due to RNA's single stranded nature in many biological systems and the substitution of ribose within the sugar phosphate backbone (instead of deoxyribose in DNA), leading to the presence of a 2′-hydroxy group within the structure of the RNA backbone. The single stranded nature of RNA and the presence of the 2′-hydroxy makes RNA susceptible to hydrolysis, in which the RNA molecule is cleaved by breaking the phosphodiester bond in the sugar-phosphate backbone, leading to degradation of the RNA molecule. Storing RNA, including storing mRNA-based vaccines or therapeutics, requires conditions that slow or prevent RNA from degrading, as described below, and interfering with the desired effects that RNA induces in targeted living cells.

Storage at extreme low temperatures below the freezing point of water, such as at or below about −20° C. or even at or below about −80° C., is known to be useful or even required for durable storage of RNA, including, but not limited to, for example, vaccines or therapeutics based on mRNA.

Storing at extreme low temperatures is more complicated than storing at more easily achieved temperatures such as temperatures approximately at the freezing point of water or refrigerated temperatures or even room temperatures or other ambient temperatures. Among the complications associated with temperatures below ambient temperatures are that refrigeration means are needed. Such refrigeration means includes cooling using thermodynamic cycles such as mechanical refrigeration (including freezers and refrigerators), frozen carbon dioxide (dry ice), or frozen water (ice).

Storing at extreme low temperatures such as at or below about −70° C. or even at or below about −80° C. or at or below about −20° C. or at or below about 4° C. complicates and adds expense to storing substances containing or based on RNA, including the storage, transportation, and therapeutic access for administration of vaccines or other RNA-based therapeutics. The complexity of using RNA is reduced the closer to room temperatures or other ambient temperatures that RNA substances can be stable.

To reduce the complexity of storing RNA substances, including mRNA and substances containing or based on mRNA such as mRNA vaccines or other therapeutic products, materials and methods for improving the stability of RNA substances so that storage can be done at temperatures greater than extremely cold temperatures are needed.

Lyophilization or freeze-drying RNA substances is used to improve the storage stability of RNA and reduce the need for storing RNA at cold temperatures or even extreme cold temperatures. However, freeze-drying and lyophilization requires specialized equipment and adds significant time, expense, and complexity to the production and storage of RNA, including but not limited to mRNA and substances containing or based on mRNA such as mRNA vaccines or other therapeutic products.

Encapsulating or complexing RNA substances with nanoparticles or lipid nanoparticles is used to improve the delivery of an RNA substance to a cell or tissue. Nanoparticles may incorporate polyethylene glycol (PEG) modified lipids, PEG conjugated lipids, or similar polymer modifications to improve nanoparticle stability. These modifications may improve the stability of the nanoparticle by decreasing aggregation and agglomeration, as well as reducing protein binding and opsonization. However, improving nanoparticle stability relates to maintaining consistent nanoparticle size distribution and retention of the encapsulated RNA within the nanoparticle as well as improving circulation half-life and reducing systemic clearance of nanoparticles following administration of the encapsulated or complexed RNA and does not necessarily improve RNA stability by preventing or usefully reducing RNA degradation during storage or shipping or reducing the need for storing or shipping RNA or nanoparticles comprising RNA at cold temperatures or even extreme cold temperatures.

### SUMMARY OF THE INVENTION

Accordingly, a primary objective of the present disclosure is to provide substances to the fields of therapeutics, diagnostics, and agriculture (including, without being limited to, both human and non-human animals and plants) that increase the stability of RNA substances and reduce degradation of RNA substances in storage environments, including storage above temperatures that require freezing or refrigeration temperatures above about −80° C., or above about −20° C., or above about 0° C., or above about 4° C., or above about 10° C.

Another primary objective of the present disclosure is to provide substances for storage environments for RNA substances and methods using substances in storage environments for RNA substances that reduces degradation of RNA substances so that RNA substances have improved stability when stored at temperatures at or above about −20° C. Another primary objective of the present disclosure is to provide substances for storage environments and methods using substances in storage environments for RNA substances that reduce degradation of RNA substances so that RNA substances have improved stability when stored at temperatures at or above about 4° C., or even at or above about 10° C.

Another primary objective of the present disclosure is to provide storage environments for RNA substances that reduce the needs for storing, transporting, distributing, or storing at a point of use such as a point for therapeutic administration (collectively “storage” or “storage and use” hereinafter) using thermodynamic cycle cooling such as vapor compression mechanical cooling or absorption cooling. Another primary objective of the present disclosure is to provide storage environments for RNA substances that reduce the needs for storage and use of RNA substances using dry ice or even frozen water ice. Another primary objective of the present disclosure is to provide storage environments for RNA substances that reduce the needs for storage and use of RNA substances at temperatures of about −80° C., or reduce the needs for storage and use of RNA substances at temperatures of about −20° C., or even about 4° C.

Another primary objective of the present disclosure is to provide compositions that may be used as storage environments for RNA substances in conjunction with cellular uptake agents (also known as transfection agents or uptake agents) that reduce the needs for storage or use of compositions at temperatures of about −80° C., or reduce the needs for storage or use of compositions at temperatures of about −20° C., or even about 4° C. As a non-limiting example RNA stabilizing substances may improve the storage or use when present in compositions comprising an RNA substance and a cellular uptake agent (such as lipids or lipid-nanoparticles (LNPs), as non-limiting examples) and improve the stability of the RNA substance in conjunction with the cellular uptake agent. As a non-limiting example, RNA stabilizing substances may improve the stability of an RNA substance in conjunction with LNPs by reducing RNA degradation in the presence of LNPs. As a non-limiting example, RNA stabilizing substances may also improve the stability of LNPs comprising RNA substances by improving LNP particle integrity or by improving the association of the RNA with the LNP or helping maintain an even LNP size distribution. As a non-limiting example, RNA stabilizing substances may also improve the stability of an RNA substance in conjunction with other cellular uptake agents, such as ionizable polymers as non-limiting examples, by reducing RNA degradation in the presence of an ionizable polymer, or reducing aggregation or agglomeration of the RNA/polymer complex.

The storage environment for RNA substances may contain at least one or more RNA stabilizing substance that is at least intimately associated with or at least partially contacting or at least partially surrounding at least one or more RNA substance achieved, such as by combining one or more RNA stabilizing substance with one or more RNA substance by mixing, pipetting, blending, submerging, vortexing, shaking, lyophilizing, vaporizing, or sublimating. The storage environment includes the immediate environment of the RNA substance such as occurs when the RNA substance is mixed or is otherwise in close association or at least partially or substantially contacting one or more RNA stabilizing substances. As a non-limiting example, the storage environment for RNA substances may be at least some RNA stabilizing substance contacting at least part of one or more RNA substances at the molecular level such as may result from submerging, blending, or mixing one or more RNA substances with the one or more RNA stabilizing substance.

The inventors have discovered that mixtures comprising selected previously known substances (that were not previously known to stabilize RNA or RNA substances) and one or more RNA substance improves RNA stability. The inventors have also discovered that RNA stability may be improved with mixtures comprising one or more RNA substance and two, three, four, five, or more selected previously known substances, not previously recognized as RNA stabilizing substances.

### Terminology

As used herein, the terms stabilize RNA or stabilizing RNA means reducing degradation of RNA substances. RNA degradation refers to cleavage of the phosphodiester backbone resulting in the reduction in molecular weight of one or more RNA molecules, such as by the loss of one or more nucleotides. As a non-limiting example, RNA degradation may be measured by the reduction in molecular weight of an RNA molecule, such as by gel electrophoresis, mass spectrometry, size exclusion chromatography, bioanalyzer, or similar methods, as non-limiting examples.

As used herein, an RNA stabilizing substance means a substance that stabilizes RNA of at least one or more RNA substance. RNA stabilizing substances provide a storage environment for the RNA substance that makes the RNA substance at least as stable at a higher temperature than the stability the RNA substance would have at a lower temperature. As a non-limiting example, an RNA stabilizing substance may provide a storage environment wherein an RNA substance may have similar stability at 4° C. as compared to the same RNA substance at −80° C. without an RNA stabilizing substance.

As used herein, a combination of one or more RNA substance with one or more RNA stabilizing substance comprises one or more RNA substance and one or more RNA stabilizing substance and the combination may include one or more additional other substances.

As used herein, the term cells includes in vivo, in vitro, in situ, or ex vivo cells, including but not limited to eukaryotic cells, prokaryotic cells, plant cells, fungal cells, insect cells, bacterial cells, mycoplasma, protozoa, plasmodium, or mammalian cells, including but not limited to the cells of vertebrate animals and the cells of humans.

As used herein, nucleic acid, is a compound or substance that comprises a polymer of nucleotides linked via a phosphodiester bond. Non-limiting examples of nucleic acids include deoxyribonucleic acid (DNA), ribonucleic acid (RNA), single-stranded, double-stranded, or hybrids thereof and may include chemical modifications or analogs thereof. Modifications may include but are not limited to modifications comprising backbone modifications, sugar modifications, or base modifications. As a non-limiting example, a polymer of nucleotides or an oligonucleotide may comprise 20 nucleotides or more linked via phosphodiester bonds.

As used herein, the term RNA means ribonucleic acid, wherein ribonucleic acid comprises a polymer of ribonucleotides linked via a phosphodiester bond, and may include chemical modifications or analogs thereof, with the exception of a chemical modification rendering the RNA into DNA. RNA may include RNA analogs, including nucleotide analogs. RNA may also include non-natural synthetic ribonucleotides. The RNA may be provided by one or more means known in the art, including but not limited to, in vitro transcription, purification from a cell or an organism, chemical synthesis, or a combination thereof. The RNA may be, but is not limited to, mRNA, rRNA, tRNA, microRNA, small interfering RNA (siRNA), self-amplifying RNA, circular RNA, small activating RNA, tmRNA, dsRNA, shRNA, snRNA, antisense RNA (asRNA), eRNA, RNA enzymes, CRISPR RNA, or total RNA. The RNA may be purified RNA (e.g., purified mRNA, purified rRNA, purified tRNA, purified microRNA, purified siRNA, purified self-amplifying RNA, purified circular RNA, purified small activating RNA, purified tmRNA, purified dsRNA, purified shRNA, purified snRNA, purified asRNA, purified eRNA, purified RNA enzymes, purified CRISPR RNA, or purified total RNA). Furthermore, RNA modifications may include sugar modifications or base modifications. As a non-limiting example, a polymer of ribonucleotides may comprise 20 nucleotides or more linked via phosphodiester bonds.

As used herein, the terms RNA substance or RNA substances means a substance or substances comprising at least one of extracellular RNA or purified extracellular RNA. RNA substance or RNA substances may include substances comprising one or more polymeric forms of RNA, including, but not limited to, single stranded or double stranded forms that may include, but are not limited to, coding or non-coding forms of RNA. RNA substance or RNA substances may also include, but are not limited to, mRNA and vaccines, therapeutics, diagnostics, or medicaments based on RNA, mRNA, or sections of RNA or other forms of ribonucleic acid that may be used for, including but not limited to, therapeutic, diagnostic, analytic, in vitro, in vivo, ex vivo, in situ, or other purposes. RNA substance or RNA substances may include, but are not limited to, mRNA, self-amplifying RNA, circular RNA, small activating RNA, IRNA, tRNA, microRNA, siRNA, tmRNA, dsRNA, shRNA, snRNA, asRNA, eRNA, RNA enzymes, CRISPR RNA, or total RNA. RNA substance or RNA substances may include, but are not limited to, purified RNA, including but not limited to, purified mRNA, purified rRNA, purified tRNA, purified microRNA, purified siRNA, purified self-amplifying RNA, purified circular RNA, purified small activating RNA, purified tmRNA, purified dsRNA, purified shRNA, purified snRNA, purified asRNA, purified eRNA, purified RNA enzymes, purified CRISPR RNA, or purified total RNA.

In some embodiments RNA substances may comprise an open reading frame. In some embodiments RNA substances may comprise at least one of a 5′ UTR, a 3′ UTR, or a poly (A)-tail, or combinations thereof (e.g. a 5′ UTR and a poly (A)-tail, or a 3′ UTR and a poly(A)-tail, or a 5′ UTR, 3′ UTR, and a poly-(A)-tail, as non-limiting examples). In some embodiments RNA substances may be polymeric single stranded RNA. In some embodiments RNA substances may be polymeric double stranded RNA. In some embodiments RNA substances may be polymeric circular RNA.

In some embodiments RNA substances may have one or more complimentary strands or partially complimentary strands, wherein a complimentary or partially complementary strand may include, but is not limited to, an RNA strand, DNA strand, peptide nucleic acid strand or other type of complementary or partially complementary strand.

In some embodiments RNA substances may comprise a coding RNA. In some embodiments RNA substances may comprise a coding RNA, wherein the coding RNA comprises a translatable region for a polypeptide. As a non-limiting example, a coding RNA may include, but is not limited to, mRNA or self-amplifying RNA.

In some embodiments RNA substances may comprise at least one of the following: mRNA or self-amplifying RNA. In some embodiments an RNA substance may comprise a translatable region for a polypeptide.

In some embodiments RNA substances may comprise a non-coding RNA. As a non-limiting example, a non-coding RNA may include, but is not limited to, microRNA, siRNA, CRISPR RNA, antisense RNA, small activating RNA, or RNA enzymes.

In some embodiments RNA substances may comprise at least one of the following: microRNA, siRNA, CRISPR RNA, antisense RNA, small activating RNA, or RNA enzymes.

In some embodiments RNA substances may comprise a polymer of at least 10 or more nucleotides, or at least 20 or more nucleotides, or at least 50 or more nucleotides, or at least 100 or more nucleotides, or at least 200 or more nucleotides, or at least 400 or more nucleotides, or at least 500 or more nucleotides, or at least 700 or more nucleotides, or at least 1000 or more nucleotides.

As used herein, the term storage environment means the substances in which an RNA substance is present (such as a composition comprising an RNA substance, as a non-limiting example), except when the RNA substance is being synthesized, produced, transcribed, or deployed for immediate use. As a non-limiting example, an RNA substance may be synthesized, at least partially purified, and then stored in an appropriate storage environment wherein the storage environment is a composition comprising at least one or more RNA stabilizing substance and one or more RNA substance.

As non-limiting examples, RNA synthesis may include chemical synthesis, transcription, in vitro transcription, enzymatic synthesis, or synthesis inside of cell (e.g. cellular synthesis or cellular transcription, including synthesis inside of an organism, plant, microbe, yeast, bacteria, eukaryotic cell, or prokaryotic cell), or other methods known in the art, or combinations thereof.

As a non-limiting example, a purified or at least partially purified RNA or RNA substance may be a polymeric RNA that has been at least partially isolated from at least one or more cellular materials or one or more components used during RNA synthesis. As a non-limiting example, a purified or at least partially purified RNA substance may be a polymeric RNA that has been at least partially isolated by removing or reducing the amount of at least one or more cellular materials (non-limiting examples including, membranes, polysaccharides, lipopolysaccharide, endotoxin, lipids, proteins, aggregates, DNA, other RNA, salts, ions, or enzymes) or one or more components used during synthesis (non-limiting examples including, unincorporated nucleotides, nucleosides, or nucleobases, ribose sugars, or amidites (e.g. phosphoramidites), or other nucleotide building blocks, or DNA, other RNA, enzymes, buffers, salts, ions, solvents, proteins, or other compounds used during synthesis).

As a non-limiting example, a purified or at least partially purified RNA or RNA substance may be a polymeric RNA that has undergone at least one or more purification steps (such as to at least partially remove or reduce the amount of one or more cellular materials or components used during RNA synthesis, as non-limiting examples). As a non-limiting example, a purified or at least partially purified RNA or RNA substance may be a polymeric RNA that has undergone at least one or more purification steps, wherein one or more purification steps may include, chromatography (including, but not limited to solid phase extraction chromatography, liquid phase extraction chromatography, supported liquid phase extraction chromatography, liquid chromatography, FPLC, HPLC, spin columns, silica or silica gel, centrifugal partition chromatography, column chromatography, reverse phase chromatography, ion exchange chromatography, cation exchange chromatography, anion exchange chromatography, size exclusion chromatography or gel-filtration chromatography, affinity chromatography, or combinations thereof), extraction (including, but not limited to, liquid-liquid extraction, phenol/chloroform extraction or extraction using organic solvents, as non-limiting examples), homogenization, treatment with an enzyme (including, but not limited to, DNase, proteinase, or specific RNase (e.g. RNase H, RNaseIII or other RNase that may degrade one or more types of RNA, as non-limiting examples)) or combinations thereof), crystallization, precipitation, dialysis, desalting, buffer exchange, centrifugation, filtration (including tangential flow filtration, vacuum filtration, or gradient or stepwise filtration, as non-limiting examples), gel electrophoresis, fractionation, evaporation, distillation, adsorption, or combinations thereof, as non-limiting examples.

As used herein, the term parenteral or parenterally means input of drugs or medications in a way not involving the intestines or the digestive tract, non-limiting examples include subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, or sublingual injection or infusion techniques.

Descriptions of substances herein may include one or more forms of the substance. Non-limiting examples of these forms may include, ionic forms, conjugate bases, conjugate acids, protonated or deprotonated forms, stereoisomers (including enantiomers or diastereomers), tautomers, salts, or combinations thereof.

As used herein, organic or organic substance means a substance comprising at least one or more carbon, wherein at least one or more carbon is covalently bonded to at least one or more hydrogen.

As used herein, inorganic or inorganic substance means a substance that does not comprise a carbon bonded to a hydrogen. A non-limiting example of an inorganic substance is an inorganic cation (K), or an inorganic salt (NaCl).

As used herein, an anion is an atom or group of atoms, comprising a negative charge at at least one pH value between about pH 5-9.

As used herein, a cation is an atom or group of atoms, comprising a positive charge at at least one pH value between about pH 4-9.

As used herein, a zwitterion comprises both a positively charged cationic moiety and a negatively charged anionic moiety at at least one pH value between about pH 5-9.

As used herein, an organosulfate comprises a sulfate group, wherein at least one oxygen of the sulfate group is covalently bonded to at least one carbon. As a non-limiting example, an organosulfate may have the formula R—O—SO₃⁻, wherein R comprises a carbon bonded to an oxygen of the sulfate group.

As used herein, an organophosphate comprises a phosphate group, wherein at least one oxygen is covalently bonded to a carbon. Furthermore, an organophosphate may comprise one oxygen of a phosphate group covalently bonded to a carbon or two oxygen atoms of a phosphate group each covalently bonded to different carbon atoms. As a non-limiting example, an organophosphate may have the formula R—O—PO₃²⁻, wherein R comprises a carbon bonded to an oxygen of the phosphate group. As another non-limiting example, an organophosphate may have the formula R—O—(PO₃⁻)—O—R′, wherein R and R′ both comprise a carbon bonded to an oxygen of the phosphate group

As used herein, a heteroatom is oxygen (O), nitrogen (N), or sulfur(S).

As used herein, alkyl refers to an acyclic branched or unbranched hydrocarbon group that lacks any double bonds; an alkyl may nevertheless be substituted to result in a substituted alkyl that may comprise (a) one or more atoms other than hydrogen and carbon and/or (b) one or more double bonds. When alkyl is methyl, which lacks any double bonds, as a non-limiting example, then alkyl may be substituted with oxo to result in formyl or alkyl may be substituted with phenyl to result in benzyl, both of which substituted alkyls comprise double bonds.

As used herein, alkenyl refers to an acyclic branched or unbranched hydrocarbon group that comprises at least one carbon-carbon double bond and that lacks any triple bonds.

As used herein, an alkyl or alkenyl substituted with hydroxy refers to the replacement of a hydrogen atom on a carbon with a hydroxy group.

As used herein, an alkyl or alkenyl substituted with amino refers to the replacement of a hydrogen atom on a carbon with an amino group.

As used herein, an alkyl or alkenyl substituted with oxo refers to the replacement of two hydrogen atoms on a single carbon with an oxygen atom resulting in a carbonyl carbon.

As used herein, an alkyl or alkenyl substituted with acetoxy refers to the replacement of a hydrogen atom on a carbon with an acetoxy group.

As used herein, one or two of hydroxy or oxo can refer to substitutions with one hydroxy, two hydroxy, one hydroxy and one oxo, one oxo, or two oxo.

As used herein, one or two of hydroxy, oxo, or amino can refer to substitutions with one hydroxy, two hydroxy, one hydroxy and one oxo, one oxo, two oxo, one amino, two amino, one hydroxy and one amino, or one oxo and one amino.

As used herein the term quaternary ammonium means a quaternary amine wherein the nitrogen is bonded to 4 carbon atoms. As a non-limiting example, a quaternary ammonium may have the formula [NR₄]⁺ wherein each R comprises a carbon atom bonded to the nitrogen atom.

As used herein, NDSB means non-detergent sulfobetaine.

As used herein, an RNA stabilizing composition is a composition comprising at least one or more RNA substance and at least one or more RNA stabilizing substance. As a non-limiting example, one or more RNA stabilizing substance may be combined with one or more RNA substance to produce an RNA stabilizing composition. As another non-limiting example, an RNA stabilizing composition comprising one or more RNA substance and one or more RNA stabilizing substance, may also comprise one or more additional substances described herein (such as one or more cellular uptake agents, additional RNA stabilizing substances, additional RNA substances, additive substances, inorganic cations (including salts comprising one or more inorganic cation), buffering agents, water, solvents, or one or more other substances described herein, as non-limiting examples).

### BRIEF DESCRIPTION OF THE DRAWINGS

For a more complete understanding of the present disclosure, and further advantages thereof, reference is now made to the following detailed description, taken in conjunction with the drawings, as described below.

FIG. 1 shows purified RNA following in vitro transcription analyzed by denaturing agarose gel electrophoresis in both A) Black and White and B) Grayscale images.

FIGS. 2A & B show the results of denaturing agarose gel electrophoresis of room temperature, 4° C., −20° C., and −80° C. stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 3A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 4A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 5A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 6A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 7A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 8A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 9A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 10A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 11A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 12A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 13A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 14A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 15A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 16A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 17A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 18A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 19A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 20A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 21A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 22A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 23A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 24A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 25A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 26A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 27A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 28A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 29A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 30A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 31A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 32A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 33A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 34A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 35A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 36A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 37A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 38A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 39A & B show the results of denaturing agarose gel electrophoresis of accelerated stability testing comparing RNA compositions to a control in both A) Black and White and B) Grayscale images in accordance with the present disclosure.

FIGS. 40A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 41A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 42A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 43A & B show the results of denaturing agarose gel electrophoresis of accelerated stability testing comparing RNA compositions to a control in both A) Black and White and B) Grayscale images in accordance with the present disclosure.

FIGS. 44A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 45A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 46A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 47A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 48A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 49A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 50A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 51A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 52A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 53A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 54A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 55A & B show the results of denaturing agarose gel electrophoresis of accelerated stability testing comparing RNA compositions to a control in both A) Black and White and B) Grayscale images in accordance with the present disclosure.

FIGS. 56A & B show the results of denaturing agarose gel electrophoresis of accelerated stability testing comparing RNA compositions to a control in both A) Black and White and B) Grayscale images in accordance with the present disclosure.

FIG. 57 shows a multi-compartment syringe loaded with components of an RNA stabilizing composition in accordance with the present disclosure.

FIG. 58 shows an RNA stabilizing composition kit in accordance with the present disclosure.

FIG. 59 is a flow chart illustrating a process for producing and using an RNA product in accordance with the present disclosure.

FIG. 60 is a flow chart illustrating a process for producing and using an RNA product in accordance with the present disclosure.

FIG. 61 shows a vial filled with components of an RNA stabilizing composition in accordance with the present disclosure.

FIG. 62 shows a single-compartment syringe filled with components of an RNA stabilizing composition in accordance with the present disclosure.

FIG. 63 shows an embedded complex with components of an RNA stabilizing composition in accordance with the present disclosure.

FIG. 64 schematically portrays an embodiment of the present disclosure comprising an RNA substance and an RNA stabilizing substance.

FIG. 65 schematically portrays an embodiment of the present disclosure comprising a chamber and an RNA substance and an RNA stabilizing substance.

FIG. 66 schematically portrays an embodiment of the present disclosure comprising a kit.

FIG. 67 schematically illustrates an embodiment of the present disclosure comprising an RNA substance and an RNA stabilizing substance.

FIGS. 68A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 69A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 70A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 71A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 72 A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 73A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 74A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 75A, B, & C each show an RNA stabilizing composition kit in accordance with the present disclosure.

FIG. 76 shows an RNA stabilizing composition kit in accordance with the present disclosure.

FIGS. 77A, B, C, D, & E each show a chamber that may be used in an RNA stabilizing composition kit in accordance with the present disclosure.

FIGS. 78A, B, C, & D each show a multi-well reservoir that may be used in an RNA stabilizing composition kit in accordance with the present disclosure.

FIG. 79 is a flow chart illustrating a process for producing and using an RNA stabilizing composition kit comprising an RNA stabilizing substance in accordance with the present disclosure.

FIG. 80 is a flow chart illustrating a process for providing and using an RNA stabilizing composition kit comprising an RNA stabilizing substance in accordance with the present disclosure.

FIGS. 81A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 82A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 83A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 84A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 85A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 86A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of room temperature stability testing comparing RNA compositions to a control in both C) Black and White and D) Grayscale images in accordance with the present disclosure.

FIGS. 87A & B show the results of denaturing agarose gel electrophoresis of room temperature stability testing comparing RNA compositions to a control in both A) Black and White and B) Grayscale images in accordance with the present disclosure.

FIGS. 88A & B show the results of denaturing agarose gel electrophoresis of room temperature stability testing comparing RNA compositions to a control in both A) Black and White and B) Grayscale images in accordance with the present disclosure.

FIGS. 89A & B show the results of denaturing agarose gel electrophoresis of room temperature stability testing comparing RNA compositions to a control in both A) Black and White and B) Grayscale images in accordance with the present disclosure.

FIGS. 90A & B show the results of denaturing agarose gel electrophoresis of room temperature stability testing comparing RNA compositions to a control in both A) Black and White and B) Grayscale images in accordance with the present disclosure.

FIGS. 91A & B show the results of denaturing agarose gel electrophoresis of accelerated stability testing of RNA with lipid nanoparticles comparing RNA compositions to a control in both A) Black and White and B) Grayscale images in accordance with the present disclosure.

FIGS. 92A & B show the results of denaturing agarose gel electrophoresis of accelerated stability testing of RNA with lipid nanoparticles comparing RNA compositions to a control in both A) Black and White and B) Grayscale images in accordance with the present disclosure.

FIGS. 93A & B show the results of denaturing agarose gel electrophoresis of room temperature stability testing of RNA with lipid nanoparticles comparing RNA compositions to a control in both A) Black and White and B) Grayscale images in accordance with the present disclosure.

FIGS. 94A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 95A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of room temperature stability testing comparing RNA compositions to a control in both C) Black and White and D) Grayscale images in accordance with the present disclosure.

FIGS. 96A & B show the results of denaturing agarose gel electrophoresis of room temperature stability testing comparing RNA compositions to a control in both A) Black and White and B) Grayscale images in accordance with the present disclosure.

FIGS. 97A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of accelerated stability testing comparing RNA compositions to a control in both C) Black and White and D) Grayscale images in accordance with the present disclosure.

FIGS. 98A & B show the results of denaturing agarose gel electrophoresis of accelerated stability testing comparing RNA compositions to a control in both A) Black and White and B) Grayscale images in accordance with the present disclosure. C & D show the results of denaturing agarose gel electrophoresis of room temperature stability testing comparing RNA compositions to a control in both C) Black and White and D) Grayscale images in accordance with the present disclosure.

FIGS. 99A & B show the results of denaturing agarose gel electrophoresis of room temperature stability testing comparing RNA compositions to a control in both A) Black and White and B) Grayscale images in accordance with the present disclosure.

### DETAILED DESCRIPTION OF THE INVENTION

Before explaining various embodiments of RNA stabilizing substances and storage environments of RNA substances in detail, it should be noted that the illustrative embodiments and examples are not limited in application or use to the details of construction and arrangement of parts or components illustrated in the accompanying drawings and description. The illustrative embodiments and examples may be implemented or incorporated in other embodiments, variations and modifications, and may be practiced or carried out in various ways. Further, unless otherwise indicated, the terms and expressions employed herein have been chosen for the purpose of describing the illustrative embodiments for the convenience of the reader and are not for the purpose of limitation thereof. Also, it will be appreciated that one or more of the following-described embodiments, expressions of embodiments and/or methods or examples, may be combined with one or more of the other following-described embodiments, expressions of embodiments and/or methods or examples.

As used herein the term “such as” followed by one or more examples or other descriptions is used to mean that the one or more examples or descriptions are non-limiting, whether or not “such as” is preceded by a comma (“,”).

As used herein the term “e.g.” followed by one or more examples or other descriptions is used to mean that the one or more examples or descriptions are non-limiting.

The headings in this section are present for convenience and are not limiting descriptions of embodiments of the present disclosure.

It is of great interest to the field of vaccines, therapeutics, diagnostics, and agriculture to increase the stability of RNA substances and reduce degradation of RNA substances. Described herein, are compositions (including pharmaceutical compositions) and methods for the design, preparation, manufacture and/or formulation of storage environments to stabilize RNA substances. Also provided are systems, processes, kits, methods, and devices for selection, design, and/or utilization of the storage environments to stabilize RNA substances described herein.

This disclosure describes newly discovered compositions that include RNA stabilizing substances that stabilize RNA and RNA substances. The present inventors have discovered RNA stabilizing substances that surprisingly stabilize RNA and RNA substances.

The present inventors have discovered that these RNA stabilizing substances may be used to create various compositions that surprisingly stabilize RNA or RNA substances. Furthermore, the present inventors have discovered that various combinations of RNA stabilizing substances surprisingly stabilize RNA or RNA substances.

The present inventors have discovered that selected RNA stabilizing substances may be used individually or as combinations of RNA stabilizing substances and that compositions comprising these RNA stabilizing substances and RNA substances may increase RNA stability. The present inventors have discovered that compositions comprising at least one or more RNA stabilizing substance and at least one or more RNA substance may increase RNA stability compared to compositions containing at least one RNA substance without at least one or more RNA stabilizing substances.

The inventors have also discovered that the stability of RNA substances may be enhanced in compositions comprising an RNA substance and one or more RNA stabilizing substance. The inventors have also discovered that the stability of RNA substances may be enhanced in an RNA storage environment comprising one or more RNA stabilizing substance.

The inventors have also discovered that RNA stabilizing substances may improve RNA stability in compositions comprising one or more RNA substance, one or more RNA stabilizing substance, and one or more cellular uptake agent (such as polymers, lipids, or lipid particles, such as LNPs, as non-limiting examples). The inventors have also discovered that RNA stabilizing substances may improve the stability of RNA in conjunction with one or more cellular uptake agent in compositions comprising one or more RNA substance, one or more RNA stabilizing substance, and one or more cellular uptake agent.

As a non-limiting example, RNA stabilizing substances may improve the storage or use when present in compositions comprising an RNA substance and a cellular uptake agent (such as lipids or lipid-nanoparticles (LNPs), as non-limiting examples) and improve the stability of the RNA substance in conjunction with the cellular uptake agent. As a non-limiting example, RNA stabilizing substances may improve the stability of an RNA substance in conjunction with LNPs by reducing RNA degradation in the presence of LNPs. As a non-limiting example, RNA stabilizing substances may also improve the stability of LNPs comprising RNA substances by improving LNP particle integrity or by improving the association of the RNA with the LNP or helping maintain an even LNP size distribution. As a non-limiting example, RNA stabilizing substances may also improve the stability of an RNA substance in conjunction with other cellular uptake agents, such as ionizable polymers as non-limiting examples, by reducing RNA degradation in the presence of an ionizable polymer, or reducing aggregation or agglomeration of the RNA/polymer complex.

Non-limiting embodiments of the present disclosure may comprise one or more RNA stabilizing substances. As a non-limiting example, one or more RNA stabilizing substances may be prepared and packaged or otherwise stored for later use in a kit comprising a package with one or more RNA stabilizing substances and labeling that may, for example, describe the contents of the kit and its intended use to be mixed with at least one RNA substance to improve the stability of the at least one RNA substance. As another non-limiting example, the one or more RNA stabilizing substance may be a component in a composition or mixture further comprising an RNA substance.

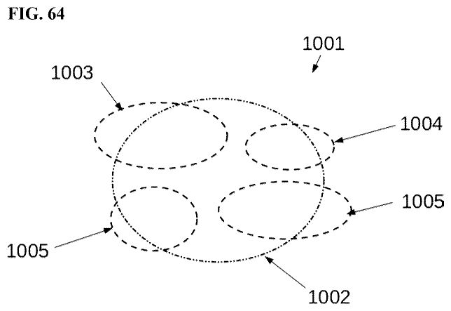

Non-limiting embodiments of the present disclosure may comprise one or more RNA substance and one or more RNA stabilizing substance. FIG. 64 schematically portrays a non-limiting example of the present disclosure comprising an RNA stabilizing substance and an RNA substance. Stabilized composition 1001 comprises at least one RNA substance 1002 and RNA stabilizing substance 1 1003. Stabilized composition 1001 may also comprise additional RNA stabilizing substances, as illustrated by RNA stabilizing substance 2 1004. Stabilizing composition 1001 may comprise property enhancing substance 1005 that enhances properties of stabilized composition 1001. As a non-limiting example, property enhancing substance 1005, may improve the biological performance of stabilized composition 1001, when stabilized composition 1001 is a medicament that will be administered to provide one or more therapies, such as property enhancing substance 1005 being a cellular uptake agent. In some embodiments of the present disclosure a composition comprising one or more RNA substance and one or more RNA stabilizing substance may be at least partially biocompatible.

Non-limiting embodiments of the present disclosure may include compositions comprising one or more RNA substance and one or more RNA stabilizing substance. Non-limiting embodiments of the present disclosure may include compositions of materials that comprise one or more RNA substance and at least one or more RNA stabilizing substance. Non-limiting embodiments of the present disclosure include compositions with improved RNA stability that may comprise one or more RNA substance and one or more RNA stabilizing substance. Non-limiting embodiments of the present disclosure may include compositions comprising one or more RNA stabilizing substance and one or more nucleic acid substance. Non-limiting embodiments of the present disclosure may include compositions comprising one or more RNA stabilizing substance and one or more DNA substance.

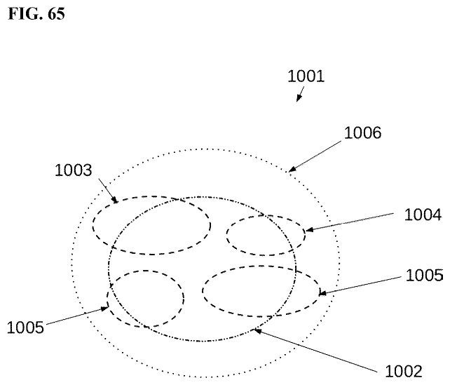

Non-limiting embodiments of the present disclosure may include compositions comprising one or more RNA substance and one or more RNA stabilizing substance in a chamber. FIG. 65 schematically portrays a non-limiting example of the present disclosure comprising an RNA stabilizing substance, an RNA containing substance, and a chamber. Stabilized composition 1001 comprises the components illustrated in FIG. 64 and those components are in chamber 1006. Stabilized composition chamber 1006 will be larger than nanoscale, as a non-limiting example, with at least one exterior dimension (e.g., length, width, thickness, diameter, perimeter) larger than about 10 micrometers. Stabilized composition chamber 1006 may be, as non-limiting examples, an ingestible capsule containing at least one RNA substance 1002 and at least one RNA stabilizing substance 1 1003 or an implantable chamber containing at least one RNA substance 1002 and at least one RNA stabilizing substance 1 1003. Chamber 1006 may be, as non-limiting examples, a hermetically sealed vial containing at least one RNA substance 1002 and at least one RNA stabilizing substance 1 1003 or a prefilled syringe containing at least one RNA substance 1002 and at least one RNA stabilizing substance 1 1003. Non-limiting embodiments of the present disclosure that improve the stability of RNA substances may comprise a storage environment comprising at least one or more vapor, liquid, powder, or solid RNA stabilizing substance.

In non-limiting example embodiments of the present disclosure, the storage environment may comprise an RNA stabilizing substance that is a liquid at the storage temperature. In non-limiting example embodiments of the present disclosure, the storage environment may comprise an RNA stabilizing substance that is a solid or gel at the storage temperature. As a non-limiting example, some substances, such as DMSO, change from being at least approximately a solid to at least approximately a liquid at its melting point of about 19° C. at one atmosphere pressure. As a non-limiting example, the storage environment may comprise a material that undergoes a phase change from solid to liquid with the solid phase condition being desired for at least part of the storage duration and the liquid phase being desired for at least another part of the storage duration. As a non-limiting example, the solid phase may provide better RNA stability than the liquid phase and the liquid phase may provide better egress or dispensing from storage chambers. As a non-limiting example, the transitions between liquid to solid or solid to liquid may help stabilize the temperature of the storage environment (and thus help maintain the temperature of RNA substances) by releasing energy when the phase changes from liquid to solid or by absorbing energy when the phase changes from solid to liquid.

As another non-limiting example, the storage environment may comprise an RNA stabilizing composition comprising water that may undergo a phase change from solid to liquid, or change physical properties, such as changing from a gel to a liquid, following the addition of a diluent comprising water (such as a buffer or water, that dilute the composition or adjust the pH, as non-limiting examples), wherein the solid or gel phase condition may be desired during storage and shipping and the liquid phase may be desired prior to use for a desired application (such as administering a vaccine or pharmaceutical composition, as non-limiting examples).

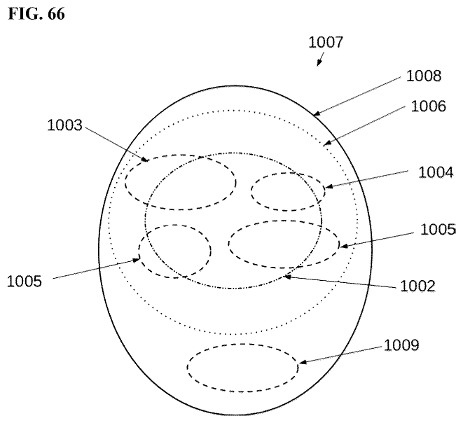

In non-limiting example embodiments of the present disclosure, components included in a composition comprising one or more RNA substance and one or more RNA stabilizing substance, may be stored separately, such as in a kit, or such as individual substances or as mixtures of one or more substance, and then combined later to produce a composition comprising one or more RNA substance and one or more RNA stabilizing substance. In non-limiting embodiments of the present disclosure, components of a composition comprising one or more RNA substance and one or more RNA stabilizing substance, may be stored together and be part of a kit comprising instructions for use or other labeling. FIG. 66 schematically portrays a non-limiting example of the present disclosure comprising an RNA stabilizing composition kit 1007. Stabilized composition kit 1007 comprises the components illustrated in FIG. 65 and those components are in kit package 1008. As non-limiting examples, kit package 1008 may be an outer wrap such as a box or bag made from a suitable material such as hard or flexible plastic or paper or cardboard. Stabilized composition kit 1007 may also comprise labeling 1009 which, as a non-limiting example, may comprise directions for use or descriptions of RNA substance 1002. Such labeling may be part of kit 1007 by, as non-limiting examples, being at least partially affixed to or inside of kit package 1008 or being part of chamber 1006 by, as non-limiting examples, being labeling affixed to or being part of chamber 1006. As a non-limiting example, a kit may comprise labeling providing storage conditions or expiration dates, or instructions for diluting or mixing one or more component of the kit prior to use, such as adding a diluent comprising water to an RNA stabilizing composition, such as a pharmaceutical composition, prior to administration to a subject.

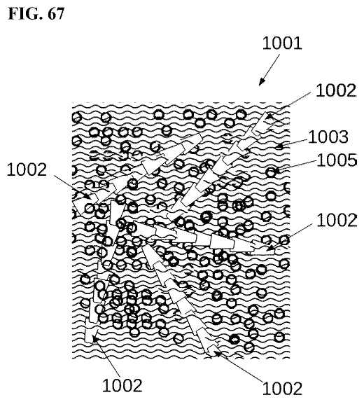

FIG. 67 schematically illustrates a non-limiting embodiment of the present disclosure. Stabilized composition 1001 comprises at least one RNA substance 1002 that at least partly contacts RNA stabilizing substance 1003. As described in more detail later, substance 1005 may be another RNA stabilizing substance or may be a substance that enhances properties of stabilized composition 1001, such as improving the biological performance of stabilized composition 1001 when it is a medicament that may be administered to provide one or more therapies. As a non-limiting example, RNA stabilizing substance 1003 may be at least part of a liquid, solution, or gel, at approximately 20° C. as a non-limiting example, that is stabilizing RNA substance 1002 that is at least substantially the same temperature as RNA stabilizing substance 1003. As a non-limiting example, stabilized composition 1001 may be part of a kit (not shown) in which the kit (not shown) comprises a chamber (not shown) containing stabilized composition 1001. In non-limiting embodiments of the present disclosure a composition comprising one or more RNA substance and one or more RNA stabilizing substance may be at least partially biocompatible.

Embodiments of the present disclosure may comprise one or more composition described herein comprising at least one RNA stabilizing substance and may also include one or more additional other substance, such as one or more cellular uptake agent, or one or more additional RNA stabilizing substance, additive substances, or water as non-limiting examples. Such embodiments may be used as at least part of pharmaceutical compositions (including medicaments or vaccines, such as, as a non-limiting example, vaccines deploying mRNA to one or more living organisms (which may include humans or may include non-human animals) with at least one RNA stabilizing substance improving the stability of the therapeutic substance and, if present, at least one cellular uptake agent improving the efficacy of the therapeutic substance.

Embodiments of the present disclosure may comprise one or more RNA stabilizing substance and one or more cellular uptake agent. Such embodiments may be used as at least part of one or more pharmaceutical compositions (including medicaments or vaccines, such as, as a non-limiting example, vaccines deploying mRNA to one or more living organisms (which may include humans or may include non-human animals) with at least one RNA stabilizing substance improving the stability of the therapeutic substance and at least one cellular uptake agent improving the efficacy of the therapeutic substance.

Non-limiting embodiments of the present disclosure may include a combination or mixture comprising one or more RNA substance and one or more RNA stabilizing substance. Non-limiting embodiments of the present disclosure that comprise one or more RNA substance and one or more RNA stabilizing substance may include combining, such as by mixing, one or more RNA substance with one or more substance that comprises at least one or more RNA stabilizing substance. In non-limiting embodiments of the present disclosure compositions with improved RNA stability may comprise a combination or mixture of one or more RNA substance and one or more RNA stabilizing substance. Non-limiting embodiments of the present disclosure that comprise one or more RNA substance and one or more RNA stabilizing substance may include combining, such as by mixing, one or more RNA substance with one or more substance that comprises at least one or more RNA stabilizing substance. In non-limiting embodiments of the present disclosure a composition comprising one or more RNA substance and one or more RNA stabilizing substance may be produced by mixing, or otherwise combining, at least one or more RNA substance and at least one or more RNA stabilizing substance. As non-limiting embodiments of the present disclosure, an RNA stabilizing composition comprising at least one or more RNA substance and one or more RNA stabilizing substance may comprise a combination or mixture of at least one or more RNA substance and at least one or more RNA stabilizing substance.

### Mechanisms and Theory

RNA is naturally unstable, including in aqueous solutions. RNA is susceptible to degradation including, but not limited to the following types of degradation: enzymatic, metal-catalyzed, hydrolysis, temperature induced, pH induced, chemically induced, oxidation induced, or radiation induced. Without being bound to any particular mechanism or mode of action, it is believed that the present disclosure provides a storage environment that increases the stability of RNA substances at warmer than extreme cold conditions by reducing the chemical reactions that degrade RNA such as by reducing exposure of RNA substances to substances within the storage environment that may induce RNA degradation.

The present inventors have discovered that RNA stabilizing substances can surprisingly increase the stability of RNA substances at temperatures above about −80° C. The present inventors have also discovered that RNA stabilizing substances can surprisingly increase the stability of RNA substances at temperatures above about −20° C., or at temperatures above about 0° C., or at temperatures above about 4° C., or at temperatures above about 10° C., or at temperatures above about 20° C., or greater.

The inventors have discovered that RNA stabilizing substances may improve the stability of RNA substances in the presence of water. Embodiments of the present disclosure may include compositions that may comprise at least one or more RNA stabilizing substance, at least one RNA substance, and water. These embodiments that may comprise water may be one or more RNA stabilizing composition described herein that may also comprise water. Embodiments of the present disclosure may comprise one or more RNA stabilizing composition described herein and may include one or more additional other substances of which water may be one of the additional other substances.

Without being bound to any particular mechanism or mode of action, RNA hydrolysis can be initiated by a base removing a proton from the 2′-OH on the ribose sugar, leading to the subsequent nucleophilic attack of the 2′ oxygen on the adjacent phosphorus atom. Base catalyzed hydrolysis activates the 2′-OH by removing the proton and creating a negatively charged 2′ oxygen and promoting nucleophilic attack of the 2′ oxygen on the adjacent phosphorus atom of the phosphodiester backbone of RNA. Water's protic nature to both donate and accept protons allows water to act as both an acid and a base at about physiologic pH. Therefore, water is capable of acting as a proton acceptor and activating the 2′-OH to promote the nucleophilic attack of the 2′ oxygen on the adjacent phosphorus atom of the phosphodiester backbone of the RNA molecule to promote RNA hydrolysis.

Without being bound to any particular mechanism or mode of action, RNA stabilizing substances of the present disclosure may at least inhibit a base removing a proton from the 2′-OH on the ribose sugar or nucleophilic attack of the 2′ oxygen on the adjacent phosphorus atom. For chemical reactions to occur reactants or reagents need to be jointly accessible and enough energy needs to be present to overcome the activation energy required for a reaction to proceed. As a non-limiting example, increasing the activation energy required for a reaction to proceed or depriving access or availability to reactants or reagents may be one mechanism by which RNA stabilizing substances improve RNA stability. Without being bound to any particular mechanism or mode of action, one or more RNA stabilizing substance may at least reduce access to the RNA substance by materials that may promote RNA hydrolysis or degradation of the RNA substance. In a non-limiting example, one or more RNA stabilizing substance in the environment of the RNA substance may create an environment that excludes water from the RNA substance or reduces the concentration of water in the environment around the RNA substance or alters the water structure or hydrogen bonding network of water or the environment around the RNA substance. Therefore, if one or more RNA stabilizing substances substantially displace all of the water in the environment of the RNA substance then the RNA substance is substantially not exposed to water, ions, or other materials that may promote RNA hydrolysis. In another non-limiting example, one or more RNA stabilizing substance in the environment of the RNA substance may also create an environment that limits the molecular mobility of water, ions, or other materials and thereby limit and/or prevent the exposure of the RNA substance to water, ions, or other materials that may promote RNA hydrolysis.

Double stranded RNA substances are more stable than single stranded RNA substances. Without being bound to any particular mechanism or mode of action, the increased stability of double stranded RNA is at least partially a result of the decreased flexibility of the double stranded RNA substance which reduces the movement of the RNA substance creating a lower probability that a 2′-OH will be in close enough proximity to an adjacent phosphorus atom to perform a nucleophilic attack and initiate RNA hydrolysis. In a non-limiting example, one or more RNA stabilizing substance that reduces the flexibility or molecular movement of a single stranded RNA substance reduces the likelihood that a 2′-OH will be in close enough proximity to an adjacent phosphorus atom to perform a nucleophilic attack and initiate RNA hydrolysis.

### RNA Stabilizing Substances

The inventors have discovered that RNA stabilizing substances may stabilize RNA substances.

The inventors have also discovered that the stability of RNA substances may be enhanced in an RNA storage environment comprising one or more RNA stabilizing substance.

The inventors have also discovered that the stability of RNA substances may be enhanced in compositions comprising an RNA substance and one or more RNA stabilizing substance.

Embodiments of the present disclosure may include a composition comprising one or more RNA substance and one or more RNA stabilizing substance.

Embodiments of the present disclosure may include a combination or mixture comprising one or more RNA substance and one or more RNA stabilizing substance.

Embodiments of the present disclosure that comprise one or more RNA substance and one or more RNA stabilizing substance may include combining, such as by mixing, one or more RNA substance with one or more substance that comprises at least one or more RNA stabilizing substance.

Embodiments of the present disclosure may include compositions of materials that comprise one or more RNA substance and at least one or more RNA stabilizing substance. The storage environment that improves the stability of RNA substances may comprise at least one or more vapor, liquid, fluid, gel, powder, or solid RNA stabilizing substance. As a non-limiting example, an RNA stabilizing composition may comprise a liquid or solution comprising one or more RNA stabilizing substance, or may comprise a gel or thixotropic fluid comprising water that also comprises one or more RNA stabilizing substance.

Embodiments of the present disclosure may include combinations of materials that comprise one or more RNA substance and at least one or more RNA stabilizing substance.

In some embodiments of the present disclosure a composition comprising one or more RNA substance and one or more RNA stabilizing substance may be produced by mixing, or otherwise combining, at least one or more RNA substance and at least one or more RNA stabilizing substance.

In some embodiments of the present disclosure a composition comprises a combination or mixture of at least one or more RNA substance and at least one or more RNA stabilizing substance.

In some embodiments of the present disclosure a composition with improved RNA stability may comprise one or more RNA substance and one or more RNA stabilizing substance.

In some embodiments of the present disclosure a composition with improved RNA stability may comprise a combination or mixture of one or more RNA substance and one or more RNA stabilizing substance.

In some embodiments of the present disclosure, each component included in a composition comprising one or more RNA substance and one or more RNA stabilizing substance, may be stored separately, such as in a kit, or such as individual substances or as mixtures of one or more substance, and then combined later to produce a composition comprising one or more RNA substance and one or more RNA stabilizing substance.

In some embodiments of the present disclosure a composition comprising one or more RNA substance and one or more RNA stabilizing substance may be at least partially biocompatible.

The inventors have also discovered that the stability of RNA substances may be enhanced in an RNA storage environment comprising multiple RNA stabilizing substances, wherein a composition may comprise an RNA substance and two or more RNA stabilizing substances, or three or more RNA stabilizing substances, or four or more RNA stabilizing substances, or five or more RNA stabilizing substances, or greater.

The inventors have also discovered that the stability of RNA substances may be enhanced in compositions comprising an RNA substance and multiple RNA stabilizing substances, wherein a composition may comprise an RNA substance and two or more RNA stabilizing substances, or three or more RNA stabilizing substances, or four or more RNA stabilizing substances, or five or more RNA stabilizing substances, or greater.

Embodiments of the present disclosure may include compositions comprising one or more RNA substance and one or more RNA stabilizing substance. Other embodiments of the present disclosure may include compositions comprising one or more RNA substance and multiple RNA stabilizing substances, wherein a composition may comprise an RNA substance and two or more RNA stabilizing substances, or three or more RNA stabilizing substances, or four or more RNA stabilizing substances, or five or more RNA stabilizing substances, or greater.

Embodiments of the present disclosure may include a combination or mixture comprising one or more RNA substance and one or more RNA stabilizing substance. Other embodiments of the present disclosure may include a combination or mixture comprising one or more RNA substance and multiple RNA stabilizing substances, wherein a combination or mixture may comprise an RNA substance and two or more RNA stabilizing substances, or three or more RNA stabilizing substances, or four or more RNA stabilizing substances, or five or more RNA stabilizing substances, or greater.

The inventors have discovered that compositions comprising at least one or more RNA substance and at least one or more RNA stabilizing substance may improve RNA stability. The inventors have also discovered that RNA stability may be improved with compositions comprising at least one or more RNA substance and multiple RNA stabilizing substances, wherein a composition may comprise an RNA substance and two or more RNA stabilizing substances, or three or more RNA stabilizing substances, or four or more RNA stabilizing substances, or five or more RNA stabilizing substances, or greater.

Embodiments comprising one or more RNA substance and one or more RNA stabilizing substance, may also comprise multiple types of RNA substances, wherein the number of different types of RNA substances may be two or more, three or more, four or more, five or more, six or more, or greater. As a non-limiting example, embodiments comprising multiple types of RNA substances, the RNA substances may be a coding RNA or non-coding RNA, or may include combinations of coding RNA and non-coding RNA.

Embodiments comprising one or more RNA substance and multiple RNA stabilizing substances, may include multiple RNA stabilizing substances from the same category of RNA stabilizing substances or different categories of RNA stabilizing substances.

In other non-limiting example embodiments, one or more RNA stabilizing composition described herein comprising a combination or mixture of one or more RNA stabilizing substance and one or more RNA substance, may also comprise one or more additional substances, including, but not limited to, one or more cellular uptake agents, additional RNA stabilizing substances, additional RNA substances, additive substances, inorganic cations (including salts comprising one or more inorganic cation), buffering agents, or water, as non-limiting examples.

In other non-limiting example embodiments, embodiments comprising a combination or mixture of one or more RNA stabilizing substance and one or more RNA substance, may also comprise one or more additional substances, including, but not limited to, one or more cellular uptake agents, additional RNA stabilizing substances, additional RNA substances, additive substances, inorganic cations (or salts thereof), buffering agents, or water, as non-limiting examples.

In some embodiments of the present disclosure is the method whereby one or more RNA stabilizing substance may be combined, such as by mixing, with at least one or more RNA substance to produce a combination or mixture comprising at least one or more RNA stabilizing substance and at least one or more RNA substance. As a non-limiting example, one or more RNA substance may be mixed, or otherwise combined, with one or more RNA stabilizing substance or multiple RNA stabilizing substances. These same methods may be used to combine or mix one or more additional substances, including, as non-limiting examples, one or more cellular uptake agents, additional RNA stabilizing substances, additional RNA substances, additive substances, inorganic cations (or salts thereof), buffering agents, or water, with one or more RNA substance and one or more RNA stabilizing substance to produce a combination or mixture comprising one or more RNA substance, one or more RNA stabilizing substance, and one or more additional substances.

In some embodiments of the present disclosure is the method whereby one or more RNA stabilizing substance may be combined, such as by mixing, with at least one or more RNA substance to produce a composition comprising at least one or more RNA substance and at least one or more RNA stabilizing substance. As a non-limiting example, one or more RNA substance may be mixed, or otherwise combined, with one or more RNA stabilizing substance or multiple RNA stabilizing substances to produce a composition comprising one or more RNA substance and one or more RNA stabilizing substance or multiple RNA stabilizing substances. These same methods may be used to mix, or otherwise combine, one or more additional substances, including, as non-limiting examples, one or more cellular uptake agents, additional RNA stabilizing substances, additional RNA substances, additive substances, inorganic cations (including salts comprising one or more inorganic cation), buffering agents, or water, with one or more RNA substance and one or more RNA stabilizing substance to produce a composition comprising one or more RNA substance, one or more RNA stabilizing substance, and one or more additional substances.

Embodiments of the present disclosure may include compositions that comprise one or more RNA stabilizing substance, at least one or more RNA substance, and water. These embodiments comprising water may include one or more RNA stabilizing composition described herein that may also comprise water.

Embodiments of the present disclosure may comprise one or more composition described herein and one or more substances that are not RNA stabilizing substances or RNA substances of which water may be one substance that is not an RNA stabilizing substance or an RNA substance.

Embodiments of the present disclosure may include compositions that comprise one or more RNA stabilizing substance, at least one or more RNA substance, and one or more cellular uptake agent. These embodiments comprising one or more cellular uptake agent may include one or more RNA stabilizing composition described herein that may also comprise one or more cellular uptake agent.

In some embodiments an RNA substance may be stored in a storage environment comprising at least one or more RNA stabilizing substance. As a non-limiting example, one or more RNA substance may stored in a storage environment comprising one or more RNA stabilizing substance, wherein the storage environment may be one or more RNA stabilizing composition described herein.

### Multiple RNA Stabilizing Substances

The inventors have discovered that combinations of RNA stabilizing substances comprising compounds from more than one RNA stabilizing substance category may be synergistic and provide better RNA stability than either individual compound. As a non-limiting example trimethylglycine (TMG) may be combined with DMSO to provide better RNA stability than either TMG or DMSO alone. Non-limiting embodiments of the present disclosure include RNA stabilizing substances that may comprise one or more compounds that are members of stabilizing substance categories as described herein. Embodiments of the present disclosure may include a composition comprising a combination or mixture of one or more RNA substance and two or more RNA stabilizing substances, wherein the RNA stabilizing substances may be from one or more different categories of RNA stabilizing substances described herein. As a non-limiting example, a composition may comprise one or more RNA substance and two or more RNA stabilizing substances, wherein the RNA stabilizing substances may be from the same or different categories of RNA stabilizing substances.

The inventors have discovered that compositions comprising at least one or more RNA substance and at least one or more RNA stabilizing substance may improve RNA stability. The inventors have discovered that RNA stability may be improved in compositions comprising at least one or more RNA substance and multiple RNA stabilizing substances such as X or more RNA stabilizing substances where X may be 2, or 3, or 4, or 5, or more than 5. As a non-limiting example, RNA stability may be improved in compositions comprising at least one or more RNA substance and multiple RNA stabilizing substances where the number of multiple RNA stabilizing substances may be five, where the RNA stabilizing substances may be from the same or different categories of RNA stabilizing substances.

Other non-limiting example embodiments of the present disclosure may include compositions that may comprise at least one RNA substance and multiple RNA stabilizing substances, in which the composition may comprise X or more RNA stabilizing substances where X may be 2, or 3, or 4, or 5, or an integer greater than 5. As a non-limiting example, an RNA stabilizing composition may comprise at least one or more RNA substance and multiple RNA stabilizing substances where the number of multiple RNA stabilizing substances may be four, where the RNA stabilizing substances may be from the same or different categories of RNA stabilizing substances.

A non-limiting example embodiment of the present disclosure may include a composition that may comprise at least one or more RNA substance and at least one or more RNA stabilizing substance that improves RNA stability. The inventors have discovered that RNA stability may be improved with compositions comprising at least one or more RNA substance and multiple RNA stabilizing substances such as the composition comprising X or more RNA stabilizing substances where X may be between two and five, or greater than five. As non-limiting example embodiments, RNA stability may be improved with compositions comprising at least one or more RNA substance and multiple RNA stabilizing substances such as comprising X RNA stabilizing substances where X may be 2, or 3, or 4, or 5, or more than 5.

### Categories of RNA Stabilizing Substances

The inventors have discovered that RNA stabilizing substances may stabilize RNA substances. Furthermore, the inventors have discovered that RNA stabilizing substances may comprise defined categories of compounds. These categories of RNA stabilizing substances may comprise defined chemical structures. Therefore, the inventors have classified RNA stabilizing substances into sets of defined chemical compounds comprising defined chemical structures. As described herein, these categories of RNA stabilizing substances may be used either individually or combined to produce a composition comprising one or more RNA stabilizing substance and one or more RNA substance.

As described below, the inventors have discovered RNA stabilizing substances comprising defined compounds from one or more of the defined chemical structures and additional defined example compounds. Embodiments of the present disclosure comprise one or more of these defined compounds. Other embodiments of the present disclosure may comprise one or more RNA substance and one or more substance of the defined compounds of RNA stabilizing substances.

The descriptions herein of RNA stabilizing substances use chemical structures, formulas, and examples to clarify the invention. For conciseness and clarity, it is understood that a person of ordinary skill in the art will understand from the disclosures herein, including the descriptions and examples herein disclosed, that RNA stabilizing substances may be made and formulated and used alone or in combinations using the disclosed descriptions. The disclosed descriptions of RNA stabilizing substances and those compounds and compositions, formulations, and uses are embodiments of the present disclosure. The different stereoisomers, tautomers, conjugate acids, and conjugate bases, of the compounds described herein are also embodiments of the present disclosure.

The inventors have surprisingly discovered, that while different categories of RNA stabilizing substances may vary in chemical formula or structure, they often share specific elements that act as a common thread. The inventors have surprisingly discovered families of compounds comprising certain moieties may be used to improve the stability of RNA substances. These specific chemical moieties are often shared across selected categories of RNA stabilizing substances.

The inventors have surprisingly discovered that adding, or otherwise combining, exogenous substances comprising mono-nucleosides or mono-nucleotides to compositions or storage environments containing RNA or RNA substances may stabilize RNA or RNA substances. The inventors have surprisingly discovered that mono-nucleoside substances or mono-nucleotide substances may stabilize RNA or RNA substances. The inventors have surprisingly discovered that substances comprising mono-nucleosides or mono-nucleotides are often shared across specific types of RNA stabilizing substances. As a non-limiting example, the inventors have surprisingly discovered that several RNA stabilizing substances may comprise mono-nucleosides or mono-nucleotides such as uridine, inosine, uridine-5′-monophosphate, inosine-5′-monophosphate, or guanosine-5′-monophosphate, as non-limiting examples.

Without being bound to any particular mechanism or mode of action, the inventors believe that substances comprising mono-nucleoside substances or mono-nucleotide substances may interact with RNA bases or unpaired nucleotides of single stranded RNA to help increase rigidity and reduce movement in flexible regions of an RNA strand that may be prone to hydrolysis.

Without being bound to any particular mechanism or mode of action, the inventors believe that substances comprising mono-nucleoside substances or mono-nucleotide substances may induce folding or interact with one or more grooves of folded RNA and help to stabilize RNA folding and prevent denaturing of RNA tertiary structure and thereby help shield RNA from ions or compounds that may promote hydrolysis of the RNA.

In some embodiments one or more exogenous mono-nucleoside substance or one or more exogenous mono-nucleotide substance may be combined with one or more RNA substance to produce a composition comprising one or more mono-nucleoside substance or one or more mono-nucleotide substance and one or more RNA substance. In some embodiments a composition may comprise one or more RNA substance and one or more exogenous mono-nucleoside substance or one or more exogenous mono-nucleotide substance. In some embodiments a composition may comprise a purified or at least partially purified RNA substance, wherein an RNA substance may be at least partially purified following synthesis, and mixed, or otherwise combined, with one or more mono-nucleoside substance or one or more mono-nucleotide substance to produce a composition comprising one or more RNA substance and one or more mono-nucleoside substance or one or more mono-nucleotide substance.

Additionally, the inventors have also discovered families of compounds comprising quaternary ammonium, tertiary sulfonium, carboxylate, sulfonate, sulfate, and phosphate groups (collectively NSCSSP compounds or NSCSSP groups or NSCSSP moieties) may be RNA stabilizing substances. Substances comprising these specified NSCSSP moieties are often shared across different categories of RNA stabilizing substances. As a non-limiting example, the inventors have surprisingly discovered that many RNA stabilizing substances often contain the following groups:

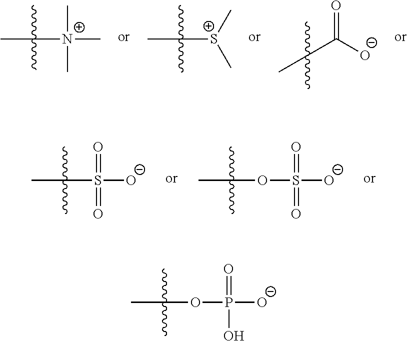

These defined groups or moieties described in this disclosure are often shared among the different types of RNA stabilizing substances and help to classify RNA stabilizing substances into several families or categories.

Without being bound to any particular mechanism or mode of action, the inventors believe that quaternary ammonium and tertiary sulfonium compounds may interact with the negatively charged phosphodiester backbone of an RNA molecule and may offer rigidity or help stabilize regions of an RNA molecule that may be prone to hydrolysis. Furthermore, quaternary ammonium and tertiary sulfonium may thereby help to shield the RNA backbone from the surrounding environment.

Without being bound to any particular mechanism or mode of action, the inventors believe that the electron resonance between oxygen atoms of carboxylate, sulfonate, sulfate, and phosphate groups may help to stabilize the water structure and hydrogen ion exchange in an aqueous solution to help prevent RNA hydrolysis. Furthermore, the negatively charged groups may help to repel the negatively charged RNA backbone or hydrogen bond with regions of RNA bases capable of being hydrogen bond donors and limit RNA flexibility in solution.

Without being bound to any particular mechanism or mode of action, the inventors have surprisingly discovered that RNA stabilizing substances comprising one or more of these specified moieties (e.g. NSCSSP groups, mono-nucleosides, or mono-nucleotides) often display synergies in improving RNA stability. While RNA stabilizing substances may improve the stability of RNA, a composition containing multiple RNA stabilizing substances may often increase the stability beyond any one RNA stabilizing substance. This synergy and greater RNA stability when more than one RNA stabilizing substance is present, may be a result of multiple RNA stabilizing substances improving RNA stability through multiple interactions that may not necessarily be achieved by a single RNA stabilizing substance.

The following detailed descriptions of RNA stabilizing substances and structures of RNA stabilizing substances often follow a common theme of compounds containing at least one or more NSCSSP group, mono-nucleoside, or mono-nucleotide. However, in some cases an RNA stabilizing substance may not contain an NSCSSP group, mono-nucleoside, or mono-nucleotide, therefore the following descriptions disclose additional embodiments and are not intended as limitations.

The inventors have discovered families of RNA stabilizing substances comprising mono-nucleoside substances, mono-nucleotide substances, or NSCSSP moieties may stabilize RNA substances in chambers larger than nanoscale and in solution without lyophilization at refrigerated temperatures and above refrigerated temperatures for hours, days, or months.

The inventors have also discovered compounds that may not comprise mono-nucleosides, mono-nucleotides, or NSCSSP moieties that stabilize RNA substances (as a non-limiting example, 3-O-ethyl-L-ascorbic acid) and those substances also have defined compositions described herein.

The inventors have also discovered that compositions comprising RNA stabilizing substances comprising mono-nucleosides or mono-nucleotides or analogs thereof and defined compositions of those RNA stabilizing substances are described herein (as non-limiting examples, uridine, inosine, uridine-5′-monosphate, inosine-5′-monosphate, or guanosine-5′-monophosphate).

The inventors have also discovered that compositions comprising RNA stabilizing substances comprising multiple NSCSSP moieties and defined compositions of those RNA stabilizing substances are described herein (as a non-limiting example, dimethylsulfoniopropionate (DMSP) or trimethylglycine (TMG)).

The inventors have also discovered that compositions comprising RNA stabilizing substances comprising modified carbohydrates comprising NSCSSP moieties and defined compositions of those RNA stabilizing substances are described herein (as a non-limiting example, phytate or diethylaminoethyl-dextran (DEAE-dextran)).

The inventors have also discovered that compositions comprising RNA stabilizing substances comprising polymers comprising NSCSSP moieties and defined compositions of those RNA stabilizing substances are described herein (as a non-limiting example, poly(2-(trimethylamino)ethyl methacrylate) (PTMAEMA)).

As described in this disclosure, the families of RNA stabilizing substances comprising mono-nucleosides, mono-nucleotides, or NSCSSP moieties are classified into categories of chemical compounds with defined compositions.

One of ordinary skill in the art would appreciate that the moieties and groups described herein (such as carboxylate, sulfate, sulfonate, and phosphate groups, as non-limiting examples) may include one or more conjugate acid or conjugate base. These moieties and groups may also include one or more associated counterions to balance one or more charges (e.g. in the form of a salt, as a non-limiting example), such as positively charged (e.g. Li, Na, K, Ca, Mg, or NH₄, or others, as non-limiting examples) or negatively charged (e.g. Cl, Br, or SO₄, or others, as non-limiting examples) counterions, as non-limiting examples. As non-limiting examples, a counterion may be an inorganic ion (such as an inorganic cation, e.g., K, as a non-limiting example) or an organic ion (such as an organic anion, e.g. tartrate, or an organic cation, e.g. choline, as non-limiting examples), or combinations thereof. Non-limiting embodiments of the present disclosure may include one or more conjugate acid or conjugate base of one or more compound or substance described herein. Non-limiting embodiments may also include one or more salt comprising one or more compound or substance described herein and one or more associated counterions. Non-limiting examples of conjugate acids or conjugate bases of one or more moieties or functional groups described herein may include, carboxylic acid (—COOH)/carboxylate (—COO⁻), or monohydrogen phosphate (—O—PO₃⁻H)/phosphate anion (—O—PO₃²⁻).

The following descriptions of structures and formulas are meant to clarify the invention and not the limitation thereof. The following formulas and structures are intended as a guide and follow standard bonding rules and may or may not include descriptions or depictions of hydrogens. In some cases, hydrogens may or may not be shown, such as hydrogens attached to carbons or protons that may be part of one or more conjugate acid, conjugate base, or tautomer, as non-limiting examples.

The compounds or substances described herein may be asymmetric (e.g., having one or more stereocenters). Embodiments of the present disclosure include stereoisomers (such as enantiomers, diastereomers, or cis-trans isomers, as non-limiting examples), of one or more compound or substance describe herein, unless stated otherwise. Compounds or substances of the present disclosure that contain asymmetrically substituted carbon atoms may be isolated in optically active or racemic forms. Methods on how to prepare optically active forms from optically active starting materials are known in the art, such as by resolution of racemic mixtures or by stereoselective synthesis. As a non-limiting example, stereoisomers of compounds or substances described herein may be isolated as a mixture of isomers or as separated isomeric forms.

The compounds or substances of the present disclosure may also include one or more tautomeric forms (e.g. tautomers). Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include keto-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms may be in equilibrium or sterically locked into one form by appropriate substitution.

Embodiments of the present disclosure may also include one or more salts (including pharmaceutically acceptable salts) of the compounds or substances described herein. Non-limiting examples of salts (including pharmaceutically acceptable salts) may include, but are not limited to, mineral or organic acid salts of basic residues (such as amines as a non-limiting example); alkali or organic salts of acidic residues (such as carboxylic acids or phosphoric acids as non-limiting examples). As non-limiting examples, salts (including pharmaceutically acceptable salts) may include one or more conventional non-toxic salts of the parent compound formed from non-toxic inorganic or organic acids, as non-limiting examples. As a non-limiting example, one or more salts or pharmaceutically acceptable salts of the present disclosure may be synthesized from a parent compound comprising a basic or acidic moiety by conventional chemical methods. As a non-limiting example, one or more salts may be prepared by reacting the free acid or base forms of a compound comprising a basic or acidic moiety with a stoichiometric amount of an appropriate base or acid in water or in an organic solvent, or in a mixture of the two, as non-limiting examples. As a non-limiting example, a salt may comprise an inorganic counterion (e.g. Na, K, or Cl, as non-limiting examples) or an organic counterion (e.g. tartrate, or choline, as non-limiting examples), or combinations thereof.

### Mono-Nucleoside and Mono-Nucleotide Section

In some embodiments an RNA stabilizing substance may comprise a mono-nucleoside substance or a mono-nucleotide substance.

In some embodiments a mono-nucleoside substance may comprise a mono-nucleoside. In some embodiments a mono-nucleoside substance may comprise a modified mono-nucleoside. In some embodiments a mono-nucleoside substance may comprise a mono-nucleoside analog.

In some embodiments a mono-nucleotide substance may comprise a mono-nucleotide. In some embodiments a mono-nucleotide substance may comprise a modified mono-nucleotide. In some embodiments a mono-nucleotide substance may comprise a mono-nucleotide analog.

In some embodiments a mono-nucleoside substance or a mono-nucleotide substance may comprise a nucleobase. In some embodiments a mono-nucleoside substance or a mono-nucleotide substance may comprise a modified nucleobase or a nucleobase analog.

As used herein, a mono-nucleoside comprises ribose bonded to a nucleobase. Non-limiting examples of a mono-nucleoside may include uridine, guanosine, or inosine.

As used herein, a mono-nucleotide comprises a mono-nucleoside (e.g. ribose bonded to a nucleobase), wherein at least one hydroxy group on the ribose sugar is substituted with a phosphate group. As a non-limiting example, at least one of the 2′-OH, 3′-OH, or 5′-OH, of a mono-nucleotide may be substituted with a phosphate group, such as monophosphate, diphosphate, triphosphate, or 2′,3′-cyclic-monophosphate, or 3′-5′-cyclic-monophosphate, as non-limiting examples. Non-limiting examples of a mono-nucleotide may include uridine-5′-monophosphate, guanosine-5′-monophosphate, or guanosine-3′,5′-cyclic-monophosphate.

In some embodiments a mono-nucleotide substance may comprise a mono-nucleotide or modified mono-nucleotide, wherein at least one hydroxy group on the ribose sugar of the mono-nucleotide may be substituted with a phosphate group. In some embodiments a mono-nucleotide substance may comprise a modified mono-nucleotide, wherein a hydroxy group on the ribose sugar of the modified mono-nucleotide may be substituted with a sulfate group in place of, or in addition to, a phosphate group. In some embodiments a mono-nucleotide substance may comprise a mono-nucleotide or modified mono-nucleotide, wherein at least one hydroxy group on the ribose sugar of the mono-nucleotide may be substituted with at least one of a sulfate group or a phosphate group.

In some embodiments a mono-nucleoside substance may comprise a modified mono-nucleoside, wherein a modified mono-nucleoside may comprise deoxy ribose instead of ribose, such as 2′-deoxyribose. In some embodiments a mono-nucleotide substance may comprise a modified mono-nucleotide, wherein a modified mono-nucleotide may comprise deoxy ribose instead of ribose, such as 2′-deoxyribose.

Modified mono-nucleosides and modified mono-nucleotides are known in the art (including mono-nucleoside and mono-nucleotide analogs or derivatives, as non-limiting examples). As non-limiting examples, modified mono-nucleosides or modified mono-nucleotides may comprise one or more modifications or substitutions to one or more canonical nucleobases. Non-limiting example modified mono-nucleosides may include: 1-methyl-pseudouridine, 5-methyl-cytidine, 7-methyl-guanosine, N6-methyl-adenosine, 8-oxo-guanosine, 1-methyl-pseudocytidine, inosine, orotidine, xanthosine, N2-methyl-isocytidine, or isocytidine. Non-limiting examples and synthesis of modified mono-nucleosides and modified mono-nucleotides are described in U.S. Pat. Nos. 9,428,535; 9,334,328; and 10,898,574, each of which are incorporated herein by reference.

Additional non-limiting examples and synthesis of nucleobase modifications are described in “Napoli, L. D., Messere, A., Montesarchio, D., Piccialli, G. & Varra, M. 6-Chloroxanthosine, a Useful Intermediate for the Efficient Syntheses of [6-15N]-Isoguanosine, Isoinosine and Other Purine Nucleoside Analogues. Nucleosides, Nucleotides & Nucleic Acids (1997) doi: 10.1080/07328319708002532.”; and “Beaussire, J.-J. & Pochet, S. Chemical and Enzymatic Synthesis of 2′-Deoxy-iso-inosine and Its Incorporation into DNA. Nucleosides and Nucleotides 14, 805-808 (1995).”, each of which are incorporated herein by reference.

In some embodiments a mono-nucleoside substance or a mono-nucleotide substance may comprise a modified mono-nucleoside or a modified mono-nucleotide. As non-limiting examples a modified mono-nucleoside or a modified mono-nucleotide may comprise one or more ribose modifications, wherein one or more hydroxy groups on the ribose sugar may be substituted with a substituent. As a non-limiting example, a mono-nucleoside substance or a mono-nucleotide substance may comprise a modified mono-nucleoside or modified mono-nucleotide comprising a 2′-deoxyribose, 2′-O-methyl-ribose, 2′,3′-dideoxyribose, or 2′-O-acetyl-ribose, as non-limiting examples.

In some embodiments an RNA stabilizing substance may comprise a mono-nucleoside substance or mono-nucleotide substance that has the formula [Formula 1-A]:

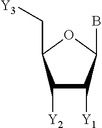

wherein:
B is a nucleobase selected from uracil, thymine, guanine, adenine, or cytosine;
Y₁-Y₃ are each independent Y groups and a Y group is selected from hydrogen (H), hydroxy (—OH), methoxy (—O—CH₃), ethoxy (—O—CH₂—CH₃), or acetoxy (—O—(C═O)—CH₃);

If the substance is a mono-nucleotide substance, then a Y group is further selected from phosphate (—O—PO₃²⁻), diphosphate ((—O—PO₂⁻)—(O—PO₃²⁻)), or triphosphate ((—O—PO₂⁻)₂—(O—PO₃²⁻)), wherein at least one Y group must be phosphate, diphosphate, or triphosphate.

In some embodiments a mono-nucleotide substance of [Formula 1-A] may comprise a Y group that is further selected from ((—O—PO₂⁻)—(O—PO₃⁻)—(CH₂)₂—N(CH₃)₃):

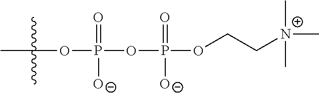 [SMILES: *OP(=O)([O-])OP(=O)([O-])OCC[N+](C)(C)C]

In some embodiments a Y group may be further selected from sulfate (—O—SO₃⁻).

In some embodiments a nucleobase B in [Formula 1-A] may be further selected from xanthine, hypoxanthine, isohypoxanthine, isoguanine, isocytosine, or orotate.

In some embodiments a mono-nucleoside substance or a mono-nucleotide substance of Formula 1-A may comprise a purine nucleobase bonded at the N9 position of the purine ring. In some embodiments a mono-nucleoside substance or mono-nucleotide substance of Formula 1-A may comprise a pyrimidine nucleobase bonded at one of the N1 position or the C5 position of the pyrimidine ring. In some embodiments a mono-nucleoside substance or mono-nucleotide substance of Formula 1-A may comprise a pyrimidine nucleobase bonded at the N1 position of the pyrimidine ring.

In some embodiments of a mono-nucleoside substance of Formula 1-A, a Y group may be selected from hydrogen (H), hydroxy (—OH), methoxy (—O—CH₃), ethoxy (—O—CH₂—CH₃), or acetoxy (—O—(C═O)—CH₃). In some embodiments of a mono-nucleoside substance of Formula 1-A, a Y group may be selected from hydrogen (H), hydroxy (—OH), methoxy (—O—CH₃), or acetoxy (—O—(C—O)—CH₃). In some embodiments of a mono-nucleoside substance of Formula 1-A, a Y group may be selected from hydrogen (H), hydroxy (—OH), or methoxy (—O—CH₃).

In some embodiments of a mono-nucleotide substance of Formula 1-A, a Y group may be selected from hydrogen (H), hydroxy (—OH), methoxy (—O—CH₃), ethoxy (—O—CH₂—CH₃), acetoxy (—O—(C═O)—CH₃), phosphate (—O—PO₃²⁻), diphosphate ((—O—PO₂⁻)—(O—PO₃²⁻)), triphosphate ((—O—PO₂⁻)₂—(O—PO₃²⁻)), or sulfate (—O—SO₃⁻). In some embodiments of a mono-nucleotide substance of Formula 1-A, a Y group may be selected from hydrogen (H), hydroxy (—OH), methoxy (—O—CH₃), ethoxy (—O—CH₂—CH₃), acetoxy (—O—(C═O)—CH₃), or phosphate (—O—PO₃²⁻). In some embodiments of a mono-nucleotide substance of Formula 1-A, a Y group may be selected from hydrogen (H), hydroxy (—OH), methoxy (—O—CH₃), or phosphate (—O—PO₃²⁻).

In some embodiments of a mono-nucleotide substance of Formula 1-A, Y₁ may be selected from hydrogen (H), hydroxy (—OH), methoxy (—O—CH₃), or acetoxy (—O—(C═O)—CH₃). In some embodiments of a mono-nucleotide substance of Formula 1-A, Y₁ may be selected from hydrogen (H), hydroxy (—OH), or methoxy (—O—CH₃). In some embodiments of a mono-nucleotide substance of Formula 1-A, Y₁ may be selected from hydrogen (H) or hydroxy (—OH).

In some embodiments of a mono-nucleotide substance of Formula 1-A, Y₂ may be selected from hydrogen (H), hydroxy (—OH), phosphate (—O—PO₃²⁻), diphosphate ((—O—PO₂⁻)—(O—PO₃²⁻)), or triphosphate ((—O—PO₂⁻)₂—(O—PO₃²⁻)). In some embodiments of a mono-nucleotide substance of Formula 1-A, Y₂ may be selected from hydrogen (H), hydroxy (—OH), or phosphate (—O—PO₃²⁻). In some embodiments of a mono-nucleotide substance of Formula 1-A, Y₂ may be selected from hydrogen (H), hydroxy (—OH), methoxy (—O—CH₃²⁻), or acetoxy (—O—(C═O)—CH₃²⁻). In some embodiments of a mono-nucleotide substance of Formula 1-A, Y₂ may be selected from hydrogen (H), hydroxy (—OH), or methoxy (—O—CH₃). In some embodiments of a mono-nucleotide substance of Formula 1-A, Y₂ may be selected from hydrogen (H), or hydroxy (—OH).

In some embodiments of a mono-nucleotide substance of Formula 1-A, Y₃ may be selected from hydrogen (H), hydroxy (—OH), phosphate (—O—PO₃²⁻), diphosphate ((—O—PO₂⁻)—(O—PO₃²⁻)), or triphosphate ((—O—PO₂⁻)₂—(O—PO₃²⁻)). In some embodiments of a mono-nucleotide substance of Formula 1-A, Y₃ may be selected from hydrogen (H), hydroxy (—OH), or phosphate (—O—PO₃²⁻). In some embodiments of a mono-nucleotide substance of Formula 1-A, Y₃ may be phosphate (—O—PO₃²⁻).

In some embodiments of a mono-nucleotide substance of Formula 1-A, a Y group comprising phosphate, diphosphate, or triphosphate, may include one or more conjugate acid or conjugate base, such as (—O—PO₃²⁻) or (—O—PO₃⁻H), as non-limiting examples.

In some embodiments of a mono-nucleoside substance of Formula 1-A, at least 2 Y groups may be hydroxy. In some embodiments of a mono-nucleoside substance of Formula 1-A, at least one Y group may be hydroxy. In some embodiments of a mono-nucleoside substance of Formula 1-A, at least 2 Y groups may be one of hydroxy or hydrogen. In some embodiments of a mono-nucleoside substance of Formula 1-A, at least three Y groups may be one of acetoxy, hydroxy, or hydrogen. In some embodiments of a mono-nucleoside substance of Formula 1-A, at least two Y groups may be one of methoxy, hydroxy, or hydrogen. In some embodiments of a mono-nucleoside substance of Formula 1-A, at least 2 Y groups may be one of hydroxy, acetoxy, or methoxy.

In some embodiments of a mono-nucleotide substance of Formula 1-A, at least 2 Y groups may be hydroxy. In some embodiments of a mono-nucleotide substance of Formula 1-A, at least one Y group may be hydroxy. In some embodiments of a mono-nucleotide substance of Formula 1-A, at least 2 Y groups may be one of hydroxy or phosphate. In some embodiments of a mono-nucleotide substance of Formula 1-A, at least one Y group may be phosphate. In some embodiments of a mono-nucleotide substance of Formula 1-A, at least 2 Y groups may one of hydroxy, hydrogen, or phosphate. In some embodiments of a mono-nucleotide substance of Formula 1-A, at least 2 Y groups may be one of hydroxy, hydrogen, phosphate, or methoxy.

A non-limiting example of a mono-nucleoside substance of [Formula 1-A] is 2′-O-methyluridine wherein B is the nucleobase uracil, Y₁ is methoxy, and Y₂ and Y₃ are hydroxy.

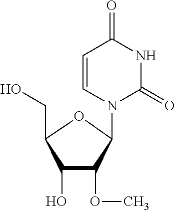

A non-limiting example of a mono-nucleotide substance of [Formula 1-A] is guanosine 5′-monophosphate wherein B is the nucleobase guanine, Y₁ and Y₂ are hydroxy, and Y₃ is phosphate.

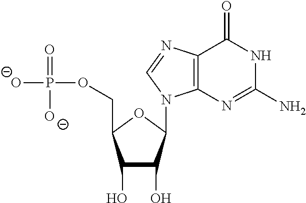

In some embodiments B in [Formula 1-A] is a nucleobase that has the formula [Formula 2-A1 or 2-A2]:

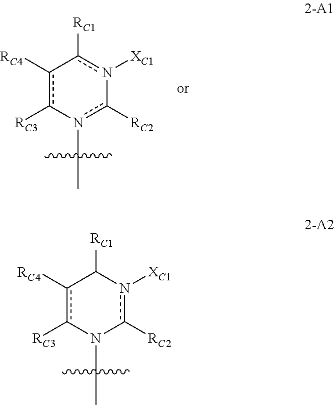

wherein:
[symbol] denotes a single or double bond;
R_{C1}-R_{C4} are each independent R_{C} groups and an R_{C} group is independently selected from hydrogen (H), hydroxy (—OH), oxo (═O), carboxylate (—COO⁻), amide ((—C═O)—NH₂)), amino (—NH₂), methylamino (—NH—CH₃), dimethylamino (—N(CH₃)₂), methyl (—CH₃), hydroxymethyl (—CH₂—OH), methoxy (—O—CH₃), ethoxy (—O—CH₂CH₃), carboxymethyl (—CH₂—(COO⁻)), or carboxymethyl ester (—(C═O)—O—CH₃);
If an R group is oxo, then the adjacent [symbol] bond within the six membered ring is a single bond;
X_{C1} is an independent X_{C} group and an X_{C} group is absent or independently selected from hydrogen (H) or methyl (—CH₃).

In some embodiments an R_{C} group may be selected from hydrogen (H), hydroxy (—OH), oxo (═O), carboxylate (—COO⁻), amide ((—C═O)—NH₂)), amino (—NH₂), methylamino (—NH—CH₃), dimethylamino (—N(CH₃)₂), methyl (—CH₃), hydroxymethyl (—CH₂—OH), methoxy (—O—CH₃), ethoxy (—O—CH₂CH₃), carboxymethyl (—CH₂—(COO⁻)) or carboxymethyl ester (—(C═O)—O—CH₃). In some embodiments an R_{C} group may be selected from hydrogen (H), hydroxy (—OH), oxo (═O), carboxylate (—COO⁻), amino (—NH₂), methylamino (—NH—CH₃), or dimethylamino (—N(CH₃)₂), methyl (—CH₃). In some embodiments an R_{C} group may be selected from hydrogen (H), hydroxy (—OH), oxo (═O), amino (—NH₂), methyl (—CH₃), or methylamino (—NH—CH₃). In some embodiments an R_{C} group may be selected from hydrogen (H), oxo (═O), amino (—NH₂), methylamino (—NH—CH₃), or dimethylamino (—N(CH₃)₂).

In some embodiments R_{C1} may be selected from hydrogen (H), oxo (═O), amino (—NH₂), methylamino (—NH—CH₃), or dimethylamino (—N(CH₃)₂). In some embodiments R_{C1} may be selected from hydrogen (H), oxo (═O), or amino (—NH₂).

In some embodiments R_{C2} may be selected from hydrogen (H), oxo (═O), amino (—NH₂), methylamino (—NH—CH₃), or dimethylamino (—N(CH₃)₂). In some embodiments R_{C2} may be selected from hydrogen (H), oxo (═O), or amino (—NH₂).

In some embodiments R_{C4} may be selected from hydrogen (H), methyl (—CH₃), hydroxymethyl (—CH₂—OH), or methoxy (—O—CH₃). In some embodiments R_{C4} may be selected from hydrogen (H), or methyl (—CH₃).

In some embodiments R_{C3} may be selected from hydrogen (H) or carboxylate (—COO⁻). In some embodiments R_{C3} may be hydrogen (H).

In some embodiments an X_{C} group may be absent or selected from hydrogen (H) or methyl (—CH₃). In some embodiments an X_{C} group may be absent or hydrogen (H).

In some embodiments X_{C1} may be absent or selected from hydrogen (H) or methyl (—CH₃). In some embodiments X_{C1} may be absent or hydrogen (H).

In some embodiments at least 1 R_{C} group may be oxo. In some embodiments at least 2 R_{C} groups may be oxo. In some embodiments at least 1 R_{C} group may be one of oxo or amino. In some embodiments at least one R_{C} group may be one of oxo or carboxylate. In some embodiments at least 2 R_{C} groups may be one of oxo, amino, methylamino, or dimethylamino. In some embodiments at least 2 R_{C} groups may be one of oxo or methyl. In some embodiments at least one X_{C} group may be one of hydrogen or methyl. In some embodiments at least one X_{C} group may be one of absent or hydrogen.

A non-limiting example of a mono-nucleotide substance of [Formula 1-A] is uridine 5′-monophosphate wherein B is a nucleobase of [Formula 2-A1], where R_{C1} and R_{C2} are oxo, R_{C3} and R_{C4} are H, X_{C1} is H, and Y₁ and Y₂ are hydroxy and Y₃ is phosphate.

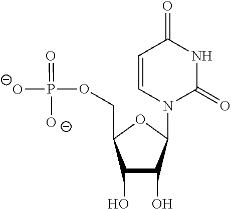

A non-limiting example of a mono-nucleotide substance of [Formula 1-A] is cytidine 3′-monophosphate wherein B is a nucleobase of [Formula 2-A1], where R_{C1} is amino, R_{C2} is oxo, R_{C3} and R_{C4} are H, X_{C1} is absent, and Y₁ and Y₃ are hydroxy and Y₂ is phosphate.

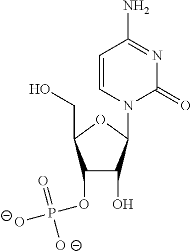

In some embodiments B in Formula 1-A is a nucleobase that has the formula [Formula 2-B1 or 2-B2]:

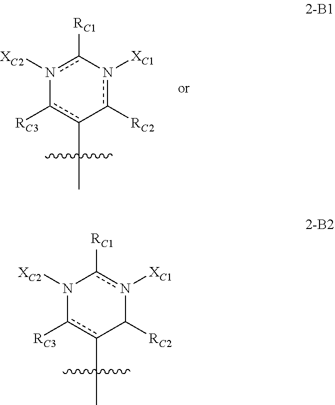

wherein:
[symbol] denotes a single or double bond;
R_{C1}-R_{C3} are each independent R_{C} groups described in this section;
If an R group is oxo, then the adjacent [symbol] bond within the six membered ring is a single bond;
X_{C1}-X_{C2} are independent X_{C} groups described in this section.

In some embodiments X_{C2} may be absent or selected from hydrogen (H) or methyl (—CH₃). In some embodiments X_{C2} may be absent or hydrogen (H).

A non-limiting example of a mono-nucleotide substance of [Formula 1-A] is N1-methyl-pseudouridine-5′-monophosphate wherein B is a nucleobase of [Formula 2-B1], where R_{C1} and R_{C2} are oxo, R_{C3} is H, X_{C1} is H, and X_{C2} is methyl, and Y₁ and Y₂ are hydroxy and Y₃ is phosphate.

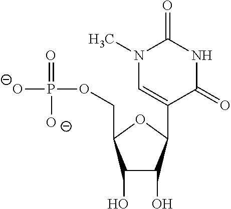

A non-limiting example of a mono-nucleotide substance of [Formula 1-A] is pseudoisocytidine-5′-monophosphate wherein B is a nucleobase of [Formula 2-B2], where R_{C1} is amino and R_{C2} is oxo, R_{C3} is H, X_{C1} is absent, and X_{C2} is H, and Y₁ and Y₂ are hydroxy and Y₃ is phosphate.

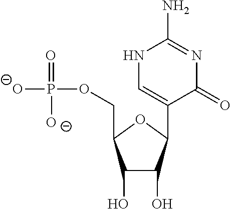

In some embodiments B in Formula 1-A is a nucleobase that has the formula [Formula 2-C1 or 2-C2]:

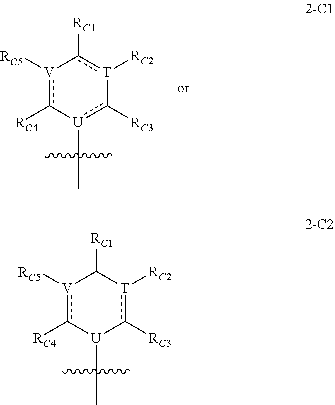

wherein:
[symbol] denotes a single or double bond;
T is independently selected from carbon (C) or nitrogen (N);
U is independently selected from carbon (C) or nitrogen (N);
V is independently selected from carbon (C) or nitrogen (N);
R_{C1}-R_{C5} are each independent R_{C} groups described in this section;
If T is nitrogen (N) then R_{C2} is absent or selected from hydrogen or methyl (—CH₃);
If V is nitrogen (N) then R_{C5} is absent or selected from hydrogen or methyl (—CH₃);
If an R group is oxo, then the adjacent [symbol] bond within the six membered ring is a single bond.

A non-limiting example of a mono-nucleotide substance of [Formula 1-A] is 1-(beta-D-ribofuranosyl)-pyridin-4-one-5′-monophosphate wherein B is a nucleobase of [Formula 2-C2], where U is N, and V and T are C, R_{C1} is oxo and R_{C2}-R_{C5} are H, and Y₁ and Y₂ are hydroxy and Y₃ is phosphate.

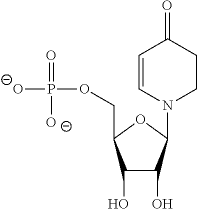

In some embodiments B in Formula 1-A is a nucleobase that has the formula [Formula 2-D1 or 2-D2]:

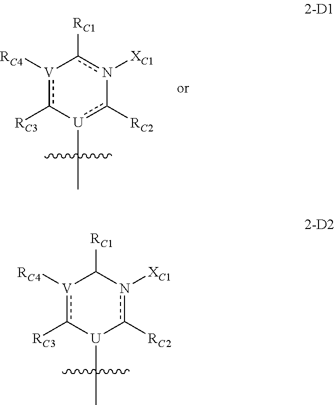

wherein:
[symbol] denotes a single or double bond;
U is independently selected from carbon (C) or nitrogen (N);
V is independently selected from carbon (C) or nitrogen (N);
R_{C1}-R_{C4} are each independent R_{C} groups described in this section;
If V is nitrogen (N) then R_{C4} is absent or selected from hydrogen or methyl (—CH₃);
If an R group is oxo, then the adjacent [symbol] bond within the six membered ring is a single bond;
X_{C1} is an independent X_{C} group described in this section.

A non-limiting example of a mono-nucleotide substance of [Formula 1-A] is 5-aza-cytidine-3′-monophosphate wherein B is a nucleobase of [Formula 2-D1], where V and U are N, R_{C1} is amino, R_{C2} is oxo, R_{C3} is H, and R_{C4} and X_{C1} are absent, and Y₁ and Y₃ are hydroxy and Y₂ is phosphate.

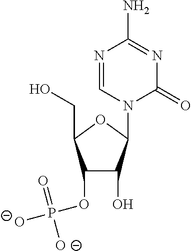

Other non-limiting examples of mono-nucleoside or mono-nucleotide substances may include the following:

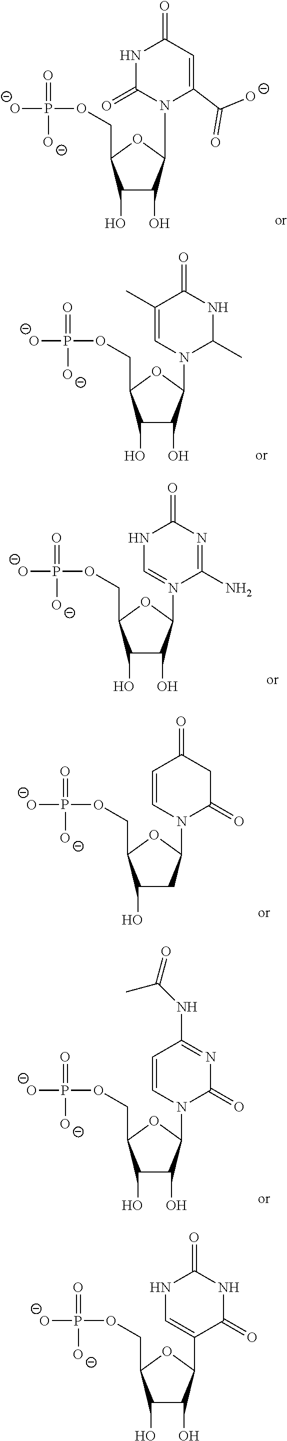

In some embodiments a mono-nucleoside substance or mono-nucleotide substance may comprise a pyrimidine nucleobase. In some embodiments a mono-nucleoside substance or mono-nucleotide substance may comprise a pyrimidine nucleobase wherein the pyrimidine nucleobase may be an unmodified or modified nucleobase. In some embodiments a mono-nucleoside substance or mono-nucleotide substance may comprise a pyrimidine nucleobase wherein the pyrimidine nucleobase may be a nucleobase analog.

In some embodiments a mono-nucleoside substance or mono-nucleotide substance may comprise a uracil nucleobase, wherein the nucleobase may a modified or unmodified nucleobase.

A mono-nucleoside or mono-nucleotide comprising a uracil nucleobase is herein referred to as a uridine-mono-nucleoside or uridine-mono-nucleotide.

In some embodiments a mono-nucleoside substance or mono-nucleotide substance may comprise a modified or unmodified uridine-mono-nucleoside or a modified or unmodified uridine-mono-nucleotide.

In some embodiments non-limiting examples of a modified or unmodified uracil nucleobase may include: uracil; 5-methyl-uracil; N3-methyl-uracil; 5-hydroxy-uracil; 5-hydroxymethyl-uracil; 5,6-dihydro-uracil; 5-carboxymethyl-uracil; 5-methoxy-uracil; or N3,5-dimethyl-uracil.

In some embodiments non-limiting examples of a modified or unmodified uridine-mono-nucleoside may include: uridine; 5-methyl-uridine; N3-methyl-uridine; 5-hydroxy-uridine; 5-hydroxymethyl-uridine; 5,6-dihydro-uridine; 5-carboxymethyl-uridine; 5-methoxy-uridine; N3,5-dimethyl-uridine; 2′-deoxyuridine; 5-methyl-2′-deoxyuridine; N3-methyl-2′-deoxyuridine; 5-hydroxy-2′-deoxyuridine; 5-hydroxymethyl-2′-deoxyuridine; 5,6-dihydro-2′-deoxyuridine; 5-carboxymethyl-2′-deoxyuridine; 5-methoxy-2′-deoxyuridine; N3,5-dimethyl-2′-deoxyuridine; 2′-O-methyluridine; 5-methyl-2′-O-methyluridine; N3-methyl-2′-O-methyluridine; 5-hydroxy-2′-O-methyluridine; 5-hydroxymethyl-2′-O-methyluridine; 5,6-dihydro-2′-O-methyluridine; 5-carboxymethyl-2′-O-methyluridine; 5-methoxy-2′-O-methyluridine; N3,5-dimethyl-2′-O-methyluridine; 2′,3′,5′-tri-O-acetyluridine; 5-methyl-2′,3′,5′-tri-O-acetyluridine; N3-methyl-2′,3′,5′-tri-O-acetyluridine; 5-hydroxy-2′,3′,5′-tri-O-acetyluridine; 5-hydroxymethyl-2′,3′,5′-tri-O-acetyluridine; 5,6-dihydro-2′,3′,5′-tri-O-acetyluridine; 5-carboxymethyl-2′,3′,5′-tri-O-acetyluridine; 5-methoxy-2′,3′,5′-tri-O-acetyluridine; or N3,5-dimethyl-2′,3′,5′-tri-O-acetyluridine.

In some embodiments non-limiting examples of a modified or unmodified uridine-mono-nucleotide may include: uridine-5′-monophosphate; 5-methyl-uridine-5′-monophosphate; N3-methyl-uridine-5′-monophosphate; 5-hydroxy-uridine-5′-monophosphate; 5-hydroxymethyl-uridine-5′-monophosphate; 5,6-dihydro-uridine-5′-monophosphate; 5-carboxymethyl-uridine-5′-monophosphate; 5-methoxy-uridine-5′-monophosphate; N3,5-dimethyl-uridine-5′-monophosphate; uridine-3′-monophosphate; 5-methyl-uridine-3′-monophosphate; N3-methyl-uridine-3′-monophosphate; 5-hydroxy-uridine-3′-monophosphate; 5-hydroxymethyl-uridine-3′-monophosphate; 5,6-dihydro-uridine-3′-monophosphate; 5-carboxymethyl-uridine-3′-monophosphate; 5-methoxy-uridine-3′-monophosphate; N3,5-dimethyl-uridine-3′-monophosphate; 2′-deoxyuridine-5′-monophosphate; 5-methyl-2′-deoxyuridine-5′-monophosphate; N3-methyl-2′-deoxyuridine-5′-monophosphate; 5-hydroxy-2′-deoxyuridine-5′-monophosphate; 5-hydroxymethyl-2′-deoxyuridine-5′-monophosphate; 5,6-dihydro-2′-deoxyuridine-5′-monophosphate; 5-carboxymethyl-2′-deoxyuridine-5′-monophosphate; 5-methoxy-2′-deoxyuridine-5′-monophosphate; N3,5-dimethyl-2′-deoxyuridine-5′-monophosphate; 2′-deoxyuridine-3′-monophosphate; 5-methyl-2′-deoxyuridine-3′-monophosphate; N3-methyl-2′-deoxyuridine-3′-monophosphate; 5-hydroxy-2′-deoxyuridine-3′-monophosphate; 5-hydroxymethyl-2′-deoxyuridine-3′-monophosphate; 5,6-dihydro-2′-deoxyuridine-3′-monophosphate; 5-carboxymethyl-2′-deoxyuridine-3′-monophosphate; 5-methoxy-2′-deoxyuridine-3′-monophosphate; or N3,5-dimethyl-2′-deoxyuridine-3′-monophosphate.

In some embodiments a mono-nucleoside substance or mono-nucleotide substance may comprise a cytosine nucleobase or isocytosine nucleobase, wherein the nucleobase may be a modified or unmodified nucleobase. As used herein, an isocytosine nucleobase comprises an amino at position 2 and an oxo at position 4 of the pyrimidine ring.

A mono-nucleoside or mono-nucleotide comprising an isocytosine nucleobase is herein referred to as an isocytidine-mono-nucleoside or isocytidine-mono-nucleotide. A mono-nucleoside or mono-nucleotide comprising a cytosine nucleobase is herein referred to as a cytidine-mono-nucleoside or cytidine-mono-nucleotide.

In some embodiments a mono-nucleoside substance or mono-nucleotide substance may comprise a modified or unmodified cytidine-mono-nucleoside or a modified or unmodified cytidine-mono-nucleotide; or a modified or unmodified isocytidine-mono-nucleoside or a modified or unmodified isocytidine-mono-nucleotide

In some embodiments non-limiting examples of a modified or unmodified cytosine nucleobase or a modified or unmodified isocytosine nucleobase may include: cytosine; N4-methyl-cytosine; N4,N4-dimethyl-cytosine; N3-methyl-cytosine; 5-methyl-cytosine; N4-acetyl-cytosine; 5-hydroxy-cytosine; 5-hydroxymethyl-cytosine; 5-methoxy-cytosine; isocytosine; N2-methyl-isocytosine; N2,N2-dimethyl-isocytosine; N3-methyl-isocytosine; 5-methyl-isocytosine; N2-acetyl-isocytosine; 5-hydroxy-isocytosine; 5-hydroxymethyl-isocytosine; or 5-methoxy-isocytosine.

In some embodiments non-limiting examples of a modified or unmodified cytidine-mono-nucleoside or a modified or unmodified isocytidine-mono-nucleoside may include: cytidine; N4-methyl-cytidine; N4,N4-dimethyl-cytidine; N3-methyl-cytidine; 5-methyl-cytidine; N4-acetyl-cytidine; 5-hydroxy-cytidine; 5-hydroxymethyl-cytidine; 5-methoxy-cytidine; 2′-deoxycytidine; N4-methyl-2′-deoxycytidine; N4,N4-dimethyl-2′-deoxycytidine; N3-methyl-2′-deoxycytidine; 5-methyl-2′-deoxycytidine; N4-acetyl-2′-deoxycytidine; 5-hydroxy-2′-deoxycytidine; 5-hydroxymethyl-2′-deoxycytidine; 5-methoxy-2′-deoxycytidine; 2′-O-methylcytidine; N4-methyl-2′-O-methylcytidine; N4,N4-dimethyl-2′-O-methylcytidine; N3-methyl-2′-O-methylcytidine; 5-methyl-2′-O-methylcytidine; N4-acetyl-2′-O-methylcytidine; 5-hydroxy-2′-O-methylcytidine; 5-hydroxymethyl-2′-O-methylcytidine; 5-methoxy-2′-O-methylcytidine; 2′,3′,5′-tri-O-acetylcytidine; N4-methyl-2′,3′,5′-tri-O-acetylcytidine; N4,N4-dimethyl-2′,3′,5′-tri-O-acetylcytidine; N3-methyl-2′,3′,5′-tri-O-acetylcytidine; 5-methyl-2′,3′,5′-tri-O-acetylcytidine; N4-acetyl-2′,3′,5′-tri-O-acetylcytidine; 5-hydroxy-2′,3′,5′-tri-O-acetylcytidine; 5-hydroxymethyl-2′,3′,5′-tri-O-acetylcytidine; 5-methoxy-2′,3′,5′-tri-O-acetylcytidine; isocytidine; N2-methyl-isocytidine; N2,N2-dimethyl-isocytidine; N3-methyl-isocytidine; 5-methyl-isocytidine; N2-acetyl-isocytidine; 5-hydroxy-isocytidine; 5-hydroxymethyl-isocytidine; 5-methoxy-isocytidine; 2′-deoxy-isocytidine; N2-methyl-2′-deoxy-isocytidine; N2,N2-dimethyl-2′-deoxy-isocytidine; N3-methyl-2′-deoxy-isocytidine; 5-methyl-2′-deoxy-isocytidine; N2-acetyl-2′-deoxy-isocytidine; 5-hydroxy-2′-deoxy-isocytidine; 5-hydroxymethyl-2′-deoxy-isocytidine; 5-methoxy-2′-deoxy-isocytidine; 2′-O-methyl-isocytidine; N2-methyl-2′-O-methyl-isocytidine; N2,N2-dimethyl-2′-O-methyl-isocytidine; N3-methyl-2′-O-methyl-isocytidine; 5-methyl-2′-O-methyl-isocytidine; N2-acetyl-2′-O-methyl-isocytidine; 5-hydroxy-2′-O-methyl-isocytidine; 5-hydroxymethyl-2′-O-methyl-isocytidine; 5-methoxy-2′-O-methyl-isocytidine; 2′,3′,5′-tri-O-acetyl-isocytidine; N2-methyl-2′,3′,5′-tri-O-acetyl-isocytidine; N2,N2-dimethyl-2′,3′,5′-tri-O-acetyl-isocytidine; N3-methyl-2′,3′,5′-tri-O-acetyl-isocytidine; 5-methyl-2′,3′,5′-tri-O-acetyl-isocytidine; N2-acetyl-2′,3′,5′-tri-O-acetyl-isocytidine; 5-hydroxy-2′,3′,5′-tri-O-acetyl-isocytidine; 5-hydroxymethyl-2′,3′,5′-tri-O-acetyl-isocytidine; or 5-methoxy-2′,3′,5′-tri-O-acetyl-isocytidine.

In some embodiments non-limiting examples of a modified or unmodified cytidine-mono-nucleotide or a modified or unmodified isocytidine-mono-nucleotide may include: cytidine-5′-monophosphate; N4-methyl-cytidine-5′-monophosphate; N4,N4-dimethyl-cytidine-5′-monophosphate; N3-methyl-cytidine-5′-monophosphate; 5-methyl-cytidine-5′-monophosphate; N4-acetyl-cytidine-5′-monophosphate; 5-hydroxy-cytidine-5′-monophosphate; 5-hydroxymethyl-cytidine-5′-monophosphate; 5-methoxy-cytidine-5′-monophosphate; cytidine-3′-monophosphate; N4-methyl-cytidine-3′-monophosphate; N4,N4-dimethyl-cytidine-3′-monophosphate; N3-methyl-cytidine-3′-monophosphate; 5-methyl-cytidine-3′-monophosphate; N4-acetyl-cytidine-3′-monophosphate; 5-hydroxy-cytidine-3′-monophosphate; 5-hydroxymethyl-cytidine-3′-monophosphate; 5-methoxy-cytidine-3′-monophosphate; 2′-deoxycytidine-5′-monophosphate; N4-methyl-2′-deoxycytidine-5′-monophosphate; N4,N4-dimethyl-2′-deoxycytidine-5′-monophosphate; N3-methyl-2′-deoxycytidine-5′-monophosphate; 5-methyl-2′-deoxycytidine-5′-monophosphate; N4-acetyl-2′-deoxycytidine-5′-monophosphate; 5-hydroxy-2′-deoxycytidine-5′-monophosphate; 5-hydroxymethyl-2′-deoxycytidine-5′-monophosphate; 5-methoxy-2′-deoxycytidine-5′-monophosphate; 2′-deoxycytidine-3′-monophosphate; N4-methyl-2′-deoxycytidine-3′-monophosphate; N4,N4-dimethyl-2′-deoxycytidine-3′-monophosphate; N3-methyl-2′-deoxycytidine-3′-monophosphate; 5-methyl-2′-deoxycytidine-3′-monophosphate; N4-acetyl-2′-deoxycytidine-3′-monophosphate; 5-hydroxy-2′-deoxycytidine-3′-monophosphate; 5-hydroxymethyl-2′-deoxycytidine-3′-monophosphate; 5-methoxy-2′-deoxycytidine-3′-monophosphate; isocytidine-5′-monophosphate; N2-methyl-isocytidine-5′-monophosphate; N2,N2-dimethyl-isocytidine-5′-monophosphate; N3-methyl-isocytidine-5′-monophosphate; 5-methyl-isocytidine-5′-monophosphate; N2-acetyl-isocytidine-5′-monophosphate; 5-hydroxy-isocytidine-5′-monophosphate; 5-hydroxymethyl-isocytidine-5′-monophosphate; 5-methoxy-isocytidine-5′-monophosphate; isocytidine-3′-monophosphate; N2-methyl-isocytidine-3′-monophosphate; N2,N2-dimethyl-isocytidine-3′-monophosphate; N3-methyl-isocytidine-3′-monophosphate; 5-methyl-isocytidine-3′-monophosphate; N2-acetyl-isocytidine-3′-monophosphate; 5-hydroxy-isocytidine-3′-monophosphate; 5-hydroxymethyl-isocytidine-3′-monophosphate; 5-methoxy-isocytidine-3′-monophosphate; 2′-deoxy-isocytidine-5′-monophosphate; N2-methyl-2′-deoxy-isocytidine-5′-monophosphate; N2,N2-dimethyl-2′-deoxy-isocytidine-5′-monophosphate; N3-methyl-2′-deoxy-isocytidine-5′-monophosphate; 5-methyl-2′-deoxy-isocytidine-5′-monophosphate; N2-acetyl-2′-deoxy-isocytidine-5′-monophosphate; 5-hydroxy-2′-deoxy-isocytidine-5′-monophosphate; 5-hydroxymethyl-2′-deoxy-isocytidine-5′-monophosphate; 5-methoxy-2′-deoxy-isocytidine-5′-monophosphate; 2′-deoxy-isocytidine-3′-monophosphate; N2-methyl-2′-deoxy-isocytidine-3′-monophosphate; N2,N2-dimethyl-2′-deoxy-isocytidine-3′-monophosphate; N3-methyl-2′-deoxy-isocytidine-3′-monophosphate; 5-methyl-2′-deoxy-isocytidine-3′-monophosphate; N2-acetyl-2′-deoxy-isocytidine-3′-monophosphate; 5-hydroxy-2′-deoxy-isocytidine-3′-monophosphate; 5-hydroxymethyl-2′-deoxy-isocytidine-3′-monophosphate; or 5-methoxy-2′-deoxy-isocytidine-3′-monophosphate.

In some embodiments a mono-nucleoside substance or mono-nucleotide substance may comprise a pseudocytosine nucleobase or pseudoisocytosine nucleobase, wherein the nucleobase may be a modified or unmodified nucleobase. As used herein, a pseudoisocytosine nucleobase comprises isocytosine, with an amino at position 2 and an oxo at position 4 of the pyrimidine ring, wherein the nucleobase may form a C5-glycoside isomer when bonded to ribose. As used herein, a pseudocytosine nucleobase comprises cytosine, with an amino at position 4 and an oxo at position 2 of the pyrimidine ring, wherein the nucleobase may form a C5-glycoside isomer when bonded to ribose.

A mono-nucleoside or mono-nucleotide comprising a pseudocytosine nucleobase is herein referred to as a pseudocytidine-mono-nucleoside or pseudocytidine-mono-nucleotide.

A mono-nucleoside or mono-nucleotide comprising a pseudoisocytosine nucleobase is herein referred to as a pseudoisocytidine-mono-nucleoside or pseudoisocytidine-mono-nucleotide.

In some embodiments a mono-nucleoside substance or mono-nucleotide substance may comprise a modified or unmodified pseudocytidine-mono-nucleoside or a modified or unmodified pseudocytidine-mono-nucleotide; or a modified or unmodified pseudoisocytidine-mono-nucleoside or a modified or unmodified pseudoisocytidine-mono-nucleotide.

In some embodiments non-limiting examples of a modified or unmodified pseudocytosine nucleobase or a modified or unmodified pseudoisocytosine nucleobase may include: pseudocytosine; N1-methyl-pseudocytosine; N3-methyl-pseudocytosine; N4-methyl-pseudocytosine; N4,N4-dimethyl-pseudocytosine; N4-acetyl-pseudocytosine; N1,N4-dimethyl-pseudocytosine; N1,N4,N4-trimethyl-pseudocytosine; pseudoisocytosine; N1-methyl-pseudoisocytosine; N3-methyl-pseudoisocytosine; N2-methyl-pseudoisocytosine; N2,N2-dimethyl-pseudoisocytosine; N2-acetyl-pseudoisocytosine; N1,N2-dimethyl-pseudoisocytosine; or N1,N2,N2-trimethyl-pseudoisocytosine.

In some embodiments non-limiting examples of a modified or unmodified pseudocytidine-mono-nucleoside or a modified or unmodified pseudoisocytidine-mono-nucleoside may include: pseudocytidine; N1-methyl-pseudocytidine; N3-methyl-pseudocytidine; N4-methyl-pseudocytidine; N4,N4-dimethyl-pseudocytidine; N4-acetyl-pseudocytidine; N1,N4-dimethyl-pseudocytidine; N1,N4,N4-trimethyl-pseudocytidine; 2′-deoxy-pseudocytidine; N1-methyl-2′-deoxy-pseudocytidine; N3-methyl-2′-deoxy-pseudocytidine; N4-methyl-2′-deoxy-pseudocytidine; N4,N4-dimethyl-2′-deoxy-pseudocytidine; N4-acetyl-2′-deoxy-pseudocytidine; N1,N4-dimethyl-2′-deoxy-pseudocytidine; N1,N4,N4-trimethyl-2′-deoxy-pseudocytidine; 2′-O-methyl-pseudocytidine; N1-methyl-2′-O-methyl-pseudocytidine; N3-methyl-2′-O-methyl-pseudocytidine; N4-methyl-2′-O-methyl-pseudocytidine; N4,N4-dimethyl-2′-O-methyl-pseudocytidine; N4-acetyl-2′-O-methyl-pseudocytidine; N1,N4-dimethyl-2′-O-methyl-pseudocytidine; N1,N4,N4-trimethyl-2′-O-methyl-pseudocytidine; 2′,3′,5′-tri-O-acetyl-pseudocytidine; N1-methyl-2′,3′,5′-tri-O-acetyl-pseudocytidine; N3-methyl-2′,3′,5′-tri-O-acetyl-pseudocytidine; N4-methyl-2′,3′,5′-tri-O-acetyl-pseudocytidine; N4,N4-dimethyl-2′,3′,5′-tri-O-acetyl-pseudocytidine; N4-acetyl-2′,3′,5′-tri-O-acetyl-pseudocytidine; N1,N4-dimethyl-2′,3′,5′-tri-O-acetyl-pseudocytidine; N1,N4,N4-trimethyl-2′,3′,5′-tri-O-acetyl-pseudocytidine; pseudoisocytidine; N1-methyl-pseudoisocytidine; N3-methyl-pseudoisocytidine; N2-methyl-pseudoisocytidine; N2,N2-dimethyl-pseudoisocytidine; N2-acetyl-pseudoisocytidine; N1,N2-dimethyl-pseudoisocytidine; N1,N2,N2-trimethyl-pseudoisocytidine; 2′-deoxy-pseudoisocytidine; N1-methyl-2′-deoxy-pseudoisocytidine; N3-methyl-2′-deoxy-pseudoisocytidine; N2-methyl-2′-deoxy-pseudoisocytidine; N2,N2-dimethyl-2′-deoxy-pseudoisocytidine; N2-acetyl-2′-deoxy-pseudoisocytidine; N1,N2-dimethyl-2′-deoxy-pseudoisocytidine; N1,N2,N2-trimethyl-2′-deoxy-pseudoisocytidine; 2′-O-methyl-pseudoisocytidine; N1-methyl-2′-O-methyl-pseudoisocytidine; N3-methyl-2′-O-methyl-pseudoisocytidine; N2-methyl-2′-O-methyl-pseudoisocytidine; N2,N2-dimethyl-2′-O-methyl-pseudoisocytidine; N2-acetyl-2′-O-methyl-pseudoisocytidine; N1,N2-dimethyl-2′-O-methyl-pseudoisocytidine; N1,N2,N2-trimethyl-2′-O-methyl-pseudoisocytidine; 2′,3′,5′-tri-O-acetyl-pseudoisocytidine; N1-methyl-2′,3′,5′-tri-O-acetyl-pseudoisocytidine; N3-methyl-2′,3′,5′-tri-O-acetyl-pseudoisocytidine; N2-methyl-2′,3′,5′-tri-O-acetyl-pseudoisocytidine; N2,N2-dimethyl-2′,3′,5′-tri-O-acetyl-pseudoisocytidine; N2-acetyl-2′,3′,5′-tri-O-acetyl-pseudoisocytidine; N1,N2-dimethyl-2′,3′,5′-tri-O-acetyl-pseudoisocytidine; or N1,N2,N2-trimethyl-2′,3′,5′-tri-O-acetyl-pseudoisocytidine.

In some embodiments non-limiting examples of a modified or unmodified pseudocytidine-mono-nucleotide or a modified or unmodified pseudoisocytidine-mono-nucleotide may include: pseudocytidine-5′-monophosphate; N1-methyl-pseudocytidine-5′-monophosphate; N3-methyl-pseudocytidine-5′-monophosphate; N4-methyl-pseudocytidine-5′-monophosphate; N4,N4-dimethyl-pseudocytidine-5′-monophosphate; N4-acetyl-pseudocytidine-5′-monophosphate; N1,N4-dimethyl-pseudocytidine-5′-monophosphate; N1,N4,N4-trimethyl-pseudocytidine-5′-monophosphate; pseudocytidine-3′-monophosphate; N1-methyl-pseudocytidine-3′-monophosphate; N3-methyl-pseudocytidine-3′-monophosphate; N4-methyl-pseudocytidine-3′-monophosphate; N4,N4-dimethyl-pseudocytidine-3′-monophosphate; N4-acetyl-pseudocytidine-3′-monophosphate; N1,N4-dimethyl-pseudocytidine-3′-monophosphate; N1,N4,N4-trimethyl-pseudocytidine-3′-monophosphate; 2′-deoxy-pseudocytidine-5′-monophosphate; N1-methyl-2′-deoxy-pseudocytidine-5′-monophosphate; N3-methyl-2′-deoxy-pseudocytidine-5′-monophosphate; N4-methyl-2′-deoxy-pseudocytidine-5′-monophosphate; N4,N4-dimethyl-2′-deoxy-pseudocytidine-5′-monophosphate; N4-acetyl-2′-deoxy-pseudocytidine-5′-monophosphate; N1,N4-dimethyl-2′-deoxy-pseudocytidine-5′-monophosphate; N1,N4,N4-trimethyl-2′-deoxy-pseudocytidine-5′-monophosphate; 2′-deoxy-pseudocytidine-3′-monophosphate; N1-methyl-2′-deoxy-pseudocytidine-3′-monophosphate; N3-methyl-2′-deoxy-pseudocytidine-3′-monophosphate; N4-methyl-2′-deoxy-pseudocytidine-3′-monophosphate; N4,N4-dimethyl-2′-deoxy-pseudocytidine-3′-monophosphate; N4-acetyl-2′-deoxy-pseudocytidine-3′-monophosphate; N1,N4-dimethyl-2′-deoxy-pseudocytidine-3′-monophosphate; N1,N4,N4-trimethyl-2′-deoxy-pseudocytidine-3′-monophosphate; pseudoisocytidine-5′-monophosphate; N1-methyl-pseudoisocytidine-5′-monophosphate; N3-methyl-pseudoisocytidine-5′-monophosphate; N2-methyl-pseudoisocytidine-5′-monophosphate; N2,N2-dimethyl-pseudoisocytidine-5′-monophosphate; N2-acetyl-pseudoisocytidine-5′-monophosphate; N1,N2-dimethyl-pseudoisocytidine-5′-monophosphate; N1,N2,N2-trimethyl-pseudoisocytidine-5′-monophosphate; pseudoisocytidine-3′-monophosphate; N1-methyl-pseudoisocytidine-3′-monophosphate; N3-methyl-pseudoisocytidine-3′-monophosphate; N2-methyl-pseudoisocytidine-3′-monophosphate; N2,N2-dimethyl-pseudoisocytidine-3′-N2-acetyl-pseudoisocytidine-3′-monophosphate; N1,N2-dimethyl-N1,N2,N2-trimethyl-pseudoisocytidine-3′-pseudoisocytidine-3′-monophosphate; monophosphate; monophosphate; 2′-deoxy-pseudoisocytidine-5′-monophosphate; N1-methyl-2′-deoxy-pseudoisocytidine-5′-monophosphate; N3-methyl-2′-deoxy-pseudoisocytidine-5′-monophosphate; N2-methyl-2′-deoxy-pseudoisocytidine-5′-monophosphate; N2,N2-dimethyl-2′-deoxy-pseudoisocytidine-5′-monophosphate; N2-acetyl-2′-deoxy-pseudoisocytidine-5′-monophosphate; N1,N2-dimethyl-2′-deoxy-pseudoisocytidine-5′-monophosphate; N1,N2,N2-trimethyl-2′-deoxy-pseudoisocytidine-5′-monophosphate; 2′-deoxy-pseudoisocytidine-3′-monophosphate; N1-methyl-2′-deoxy-pseudoisocytidine-3′-monophosphate; N3-methyl-2′-deoxy-pseudoisocytidine-3′-monophosphate; N2-methyl-2′-deoxy-pseudoisocytidine-3′-monophosphate; N2,N2-dimethyl-2′-deoxy-pseudoisocytidine-3′-monophosphate; N2-acetyl-2′-deoxy-pseudoisocytidine-3′-monophosphate; N1,N2-dimethyl-2′-deoxy-pseudoisocytidine-3′-monophosphate; or N1,N2,N2-trimethyl-2′-deoxy-pseudoisocytidine-3′-monophosphate.

In some embodiments a mono-nucleoside substance or mono-nucleotide substance may comprise a pseudouracil nucleobase, wherein the nucleobase may be a modified or unmodified nucleobase. As used herein, a pseudouracil nucleobase comprises uracil, with an oxo at position 2 and position 4 of the pyrimidine ring, wherein the nucleobase may form a C5-glycoside isomer when bonded to ribose.

A mono-nucleoside or mono-nucleotide comprising a pseudouracil nucleobase is herein referred to as a pseudouridine-mono-nucleoside or pseudouridine-mono-nucleotide.

In some embodiments a mono-nucleoside substance or mono-nucleotide substance may comprise a modified or unmodified pseudouridine-mono-nucleoside or a modified or unmodified pseudouridine-mono-nucleotide.

In some embodiments non-limiting examples of a modified or unmodified pseudouracil nucleobase may include: pseudouracil; N1-methyl-pseudouracil; N3-methyl-pseudouracil; or N1,N3-dimethyl-pseudouracil.

In some embodiments non-limiting examples of a modified or unmodified pseudouridine-mono-nucleoside may include: pseudouridine; N1-methyl-pseudouridine; N3-methyl-pseudouridine; N1,N3-dimethyl-pseudouridine; 2′-deoxy-pseudouridine; N1-methyl-2′-deoxy-pseudouridine; N3-methyl-2′-deoxy-pseudouridine; N1,N3-dimethyl-2′-deoxy-pseudouridine; 2′-O-methyl-pseudouridine; N1-methyl-2′-O-methyl-pseudouridine; N3-methyl-2′-O-methyl-pseudouridine; N1,N3-dimethyl-2′-O-methyl-pseudouridine; 2′,3′,5′-tri-O-acetyl-pseudouridine; N1-methyl-2′,3′,5′-tri-O-acetyl-pseudouridine; N3-methyl-2′,3′,5′-tri-O-acetyl-pseudouridine; or N1,N3-dimethyl-2′,3′,5′-tri-O-acetyl-pseudouridine.

In some embodiments non-limiting examples of a modified or unmodified pseudouridine-mono-nucleotide may include: pseudouridine-5′-monophosphate; N1-methyl-pseudouridine-5′-monophosphate; N3-methyl-pseudouridine-5′-monophosphate; N1,N3-dimethyl-pseudouridine-5′-monophosphate; pseudouridine-3′-monophosphate; N1-methyl-pseudouridine-3′-monophosphate; N3-methyl-pseudouridine-3′-monophosphate; N1,N3-dimethyl-pseudouridine-3′-monophosphate; 2′-deoxy-pseudouridine-5′-monophosphate; N1-methyl-2′-deoxy-pseudouridine-5′-monophosphate; N3-methyl-2′-deoxy-pseudouridine-5′-monophosphate; N1,N3-dimethyl-2′-deoxy-pseudouridine-5′-monophosphate; 2′-deoxy-pseudouridine-3′-monophosphate; N1-methyl-2′-deoxy-pseudouridine-3′-monophosphate; N3-methyl-2′-deoxy-pseudouridine-3′-monophosphate; or N1,N3-dimethyl-2′-deoxy-pseudouridine-3′-monophosphate.

In some embodiments a mono-nucleoside substance or mono-nucleotide substance may comprise an orotate nucleobase, wherein the nucleobase may be a modified or unmodified nucleobase. As used herein, an orotate nucleobase comprises orotate (also known as orotic acid), wherein the nucleobase may form an N-glycosidic bond with ribose at the N1 position of the pyrimidine ring.

In some embodiments a mono-nucleoside substance or mono-nucleotide substance may comprise a modified or unmodified orotidine-mono-nucleoside or a modified or unmodified orotidine-mono-nucleotide.

A mono-nucleoside or mono-nucleotide comprising an orotate nucleobase is herein referred to as an orotidine-mono-nucleoside or orotidine-mono-nucleotide.

In some embodiments non-limiting examples of a modified or unmodified orotate nucleobase may include: orotate; 3-methyl-orotate; or 5,6-dihydro-orotate.

In some embodiments non-limiting examples of a modified or unmodified orotidine-mono-nucleoside may include: orotidine; 3-methyl-orotidine; 5,6-dihydro-orotidine; 2′-deoxy-orotidine; 3-methyl-2′-deoxy-orotidine; 5,6-dihydro-2′-deoxy-orotidine; 2′-O-methyl-orotidine; 3-methyl-2′-O-methyl-orotidine; 5,6-dihydro-2′-O-methyl-orotidine; 2′,3′,5′-tri-O-acetyl-orotidine; 3-methyl-2′,3′,5′-tri-O-acetyl-orotidine; or 5,6-dihydro-2′,3′,5′-tri-O-acetyl-orotidine.

In some embodiments non-limiting examples of a modified or unmodified orotidine-mono-nucleotide may include: orotidine-5′-monophosphate; 3-methyl-orotidine-5′-monophosphate; 5,6-dihydro-orotidine-5′-monophosphate; orotidine-3′-monophosphate; 3-methyl-orotidine-3′-monophosphate; 5,6-dihydro-orotidine-3′-monophosphate; 2′-deoxy-orotidine-5′-monophosphate; 3-methyl-2′-deoxy-orotidine-5′-monophosphate; 5,6-dihydro-2′-deoxy-orotidine-5′-monophosphate; 2′-deoxy-orotidine-3′-monophosphate; 3-methyl-2′-deoxy-orotidine-3′-monophosphate; or 5,6-dihydro-2′-deoxy-orotidine-3′-monophosphate.

In some embodiments a mono-nucleoside substance or mono-nucleotide substance may comprise a thymine nucleobase, wherein a thymine nucleobase may also be known as 5-methyl-uracil.

In some embodiments a mono-nucleoside substance may comprise a thymidine-mono-nucleoside (e.g. thymidine), wherein thymidine is also known as 5-methyl-2′-deoxyuridine. In some embodiments a mono-nucleotide substance may comprise a thymidine-mono-nucleotide (e.g. thymidine-5′-monophosphate), wherein thymidine-5′-monophosphate is also known as 5-methyl-2′-deoxyuridine-5′-monophosphate.

In some embodiments a mono-nucleoside substance or mono-nucleotide substance may comprise a 2-pyrimidinone (also known as 2-hydroxypyrimidine) nucleobase or 4-pyrimidinone (also known as 4-hydroxypyrimidine) nucleobase, wherein the nucleobase may form an N-glycosidic bond with ribose at the N1 position of the pyrimidine ring.

In some embodiments a mono-nucleoside substance or mono-nucleotide substance may comprise a modified or unmodified pyrimidin-2-one-mono-nucleoside or a modified or unmodified pyrimidin-2-one-mono-nucleotide; or a modified or unmodified pyrimidin-4-one-mono-nucleoside or a modified or unmodified pyrimidin-4-one-mono-nucleotide.

A mono-nucleoside or mono-nucleotide comprising a 2-pyrimidinone nucleobase is herein referred to as a pyrimidin-2-one-mono-nucleoside or pyrimidin-2-one-mono-nucleotide.

A mono-nucleoside or mono-nucleotide comprising a 4-pyrimidinone nucleobase is herein referred to as a pyrimidin-4-one-mono-nucleoside or pyrimidin-4-one-mono-nucleotide.

In some embodiments a mono-nucleoside substance or mono-nucleotide substance may comprise a pyridine nucleobase, wherein the pyridine nucleobase may form an N-glycosidic bond with ribose at the N1 position of the pyridine ring.

In some embodiments a mono-nucleoside substance or mono-nucleotide substance may comprise a modified or unmodified pyridine-mono-nucleoside or a modified or unmodified pyridine-mono-nucleotide.

A mono-nucleoside or mono-nucleotide comprising a pyridine-nucleobase is herein referred to as a pyridine-mono-nucleoside or pyridine-mono-nucleotide.

In some embodiments non-limiting examples of a pyridine-nucleobase may include: 2-pyridone (also known as 2-hydroxypyridine), 4-pyridone (also known as 4-hydroxypyridine), or 2,4-dihydroxypyridine (also known as 2,4-pyridinediol, or 3-deazauracil).

In some embodiments non-limiting examples of other nucleobases may include: 2-pyrimidinone or 4-pyrimidinone.

In some embodiments non-limiting examples of other mono-nucleoside substances may include: 1-(beta-D-ribofuranosyl)-pyrimidin-2-one (also known as zebularine); 1-(beta-D-ribofuranosyl)-pyrimidin-4-one; 1-(beta-D-ribofuranosyl)-pyridin-2-one; 1-(beta-D-ribofuranosyl)-pyridin-4-one; 1-(beta-D-ribofuranosyl)-pyridin-2,4-dione; pyrrolocytidine; wyosine; wybutosine; 1-(2-deoxy-beta-D-ribofuranosyl)-pyrimidin-2-one (also known as 2′-deoxy-zebularine); 1-(2-deoxy-beta-D-ribofuranosyl)-pyrimidin-4-one; 1-(2-deoxy-beta-D-ribofuranosyl)-pyridin-2-one; 1-(2-deoxy-beta-D-ribofuranosyl)-pyridin-4-one; 1-(2-deoxy-beta-D-ribofuranosyl)-pyridin-2,4-dione; 2′-deoxy-pyrrolocytidine; 2′-deoxy-wyosine; or 2′-deoxy-wybutosine.

In some embodiments non-limiting examples of other mono-nucleotide substances may include: citicoline (also known as cytidine diphosphate-choline, CDP-choline, or cytidine 5′-diphosphocholine); guanosine-3′,5′-cyclic-monophosphate; guanosine-2′,3′-cyclic-monophosphate; 2′-deoxyguanosine-3′,5′-cyclic-monophosphate; isoguanosine-3′,5′-cyclic-monophosphate; isoguanosine-2′,3′-cyclic-monophosphate; 2′-deoxy-isoguanosine-3′,5′-cyclic-monophosphate; inosine-3′,5′-cyclic-monophosphate; inosine-2′,3′-cyclic-monophosphate; 2′-deoxyinosine-3′,5′-cyclic-monophosphate; isoinosine-3′,5′-cyclic-monophosphate; isoinosine-2′,3′-cyclic-monophosphate; 2′-deoxy-isoinosine-3′,5′-cyclic-monophosphate; uridine-3′,5′-cyclic-monophosphate; uridine-2′,3′-cyclic-monophosphate; 2′-deoxyuridine-3′,5′-cyclic-monophosphate; xanthosine-3′,5′-cyclic-monophosphate; xanthosine-2′,3′-cyclic-monophosphate; 2′-deoxyxanthosine-3′,5′-cyclic-monophosphate; 1-(beta-D-ribofuranosyl)-pyrimidin-2-one-5′-monophosphate (also known as zebularine-5′-monophosphate); 1-(beta-D-ribofuranosyl)-pyrimidin-4-one-5′-monophosphate; 1-(beta-D-ribofuranosyl)-pyridin-2-one-5′-monophosphate; 1-(beta-D-ribofuranosyl)-pyridin-4-one-5′-monophosphate; 1-(beta-D-ribofuranosyl)-pyridin-2,4-dione-5′-monophosphate; pyrrolocytidine-5′-monophosphate; wyosine-5′-monophosphate; wybutosine-5′-monophosphate; 1-(2-deoxy-beta-D-ribofuranosyl)-pyrimidin-2-one-5′-monophosphate (also known as 2′-deoxy-zebularine-5′-monophosphate); 1-(2-deoxy-beta-D-ribofuranosyl)-pyrimidin-4-one-5′-monophosphate; 1-(2-deoxy-beta-D-ribofuranosyl)-pyridin-2-one-5′-monophosphate; 1-(2-deoxy-beta-D-ribofuranosyl)-pyridin-4-one-5′-monophosphate; 1-(2-deoxy-beta-D-ribofuranosyl)-pyridin-2,4-dione-5′-monophosphate; 2′-deoxy-pyrrolocytidine-5′-monophosphate; 2′-deoxy-wyosine-5′-monophosphate; or 2′-deoxy-wybutosine-5′-monophosphate.

In some embodiments B in [Formula 1-A] is a nucleobase that has the formula [Formula 2-E]:

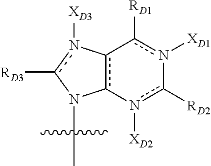

wherein:
[symbol] denotes a single or double bond;
R_{P1}-R_{P3} are each independent R_{P} groups and an R_{P} group is independently selected from hydrogen (H), hydroxy (—OH), oxo (═O), amino (—NH₂), methylamino (—NH—CH₃), dimethylamino (—N(CH₃)₂), methyl (—CH₃), hydroxymethyl (—CH₂—OH), methoxy (—O—CH₃), ethoxy (—O—CH₂CH₃), or carboxymethyl ester (—(C═O)—O—CH₃);
If an R group is oxo, then the adjacent [symbol] bond within the five or six membered ring is a single bond;
X_{D1}-X_{D3} are independent X_{D} groups and an X_{D} group is absent or independently selected from hydrogen (H) or methyl (—CH₃).

In some embodiments an R_{P} group may be selected from hydrogen (H), hydroxy (—OH), oxo (═O), amino (—NH₂), methylamino (—NH—CH₃), dimethylamino (—N(CH₃)₂), methyl (—CH₃), hydroxymethyl (—CH₂—OH), methoxy (—O—CH₃), ethoxy (—OCH₂CH₃), or carboxymethyl ester (—(C—O)—O—CH₃). In some embodiments an R_{P} group may be selected from hydrogen (H), hydroxy (—OH), oxo (═O), amino (—NH₂), methylamino (—NH—CH₃), dimethylamino (—N(CH₃)₂), or methyl (—CH₃). In some embodiments an R_{P} group may be selected from hydrogen (H), oxo (═O), amino (—NH₂), methylamino (—NH—CH₃), or dimethylamino (—N(CH₃)₂).

In some embodiments an X_{D} group may be absent or selected from hydrogen (H) or methyl (—CH₃). In some embodiments an X_{D} group may be absent or selected from hydrogen (H).

In some embodiments R_{P1} may be selected from hydrogen (H), oxo (═O), amino (—NH₂), methylamino (—NH—CH₃), or dimethylamino (—N(CH₃)₂). In some embodiments R_{P1} may be selected from hydrogen (H), oxo (═O), or amino (—NH₂).

In some embodiments R_{P2} may be selected from hydrogen (H), oxo (═O), amino (—NH₂), methylamino (—NH—CH₃), or dimethylamino (—N(CH₃)₂). In some embodiments R_{P2} may be selected from hydrogen (H), oxo (═O), or amino (—NH₂).

In some embodiments R_{P3} may be selected from hydrogen (H), oxo (═O), or hydroxy (—OH). In some embodiments R_{P3} may be selected from hydrogen (H), or oxo (═O). In some embodiments R_{P3} may be hydrogen (H).

In some embodiments X_{D1} may be absent or selected from hydrogen (H) or methyl (—CH₃). In some embodiments X_{D1} may be absent or selected from hydrogen (H).

In some embodiments X_{D2} may be absent or selected from hydrogen (H) or methyl (—CH₃). In some embodiments X_{D2} may be absent or selected from hydrogen (H).

In some embodiments X_{D3} may be absent or selected from hydrogen (H) or methyl (—CH₃). In some embodiments X_{D3} may be absent or selected from hydrogen (H).

In some embodiments at least 2 R_{P} groups may be one of oxo, amino, methylamino, or dimethylamino. In some embodiments at least 1 R_{P} group may be oxo. In some embodiments at least 2 R_{P} groups may be one of hydrogen or oxo. In some embodiments at least 2 R_{P} groups may be one of hydrogen, oxo, or amino. In some embodiments at least 2 R_{P} groups may be one of hydrogen, oxo, hydroxy, or amino. In some embodiments at least 2 X_{D} groups may be absent or hydrogen. In some embodiments at least 2 X_{D} groups may be one of hydrogen or methyl.

A non-limiting example of a mono-nucleoside substance of [Formula 1-A] is inosine wherein B is a nucleobase of [Formula 2-E], where R_{P1} is Oxo, R_{P2} and R_{P3} are H, X_{D1} is H, X_{D2} and X_{D3} are absent, and Y₁-Y₃ are hydroxy.

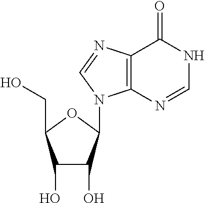

A non-limiting example of a mono-nucleotide substance of [Formula 1-A] is N6-methyladenosine-5′-monophosphate wherein B is a nucleobase of [Formula 2-E], where R_{P1} is methylamino, R_{P2} and R_{P3} are H, X_{D1}, X_{D2} and X_{D3} are absent, and Y₁ and Y₂ are hydroxy, and Y₃ is phosphate.

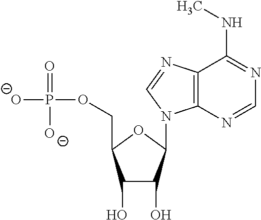

In some embodiments B in [Formula 1-A] is a nucleobase that has the formula [Formula 2-F]:

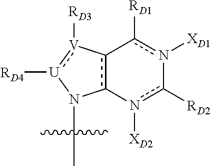

wherein:
[symbol] denotes a single or double bond;
V is selected from carbon (C) or nitrogen (N);
U is selected from carbon (C) or nitrogen (N);
If V is nitrogen (N) then U is carbon (C);
If U is nitrogen (N) then V is carbon (C);
If V is carbon (C) then U is selected from carbon (C) or nitrogen (N);
R_{P1}-R_{P4} are each independent R_{P} groups described in this section;
If V is nitrogen (N) then R_{P3} is absent or selected from hydrogen or methyl (—CH₃);
If U is nitrogen (N) then R_{P4} is absent or selected from hydrogen or methyl (—CH₃);
If an R group is oxo, then the adjacent [symbol] bond within the five or six membered ring is a single bond;
X_{D1}-X_{D2} are independent X_{D} groups described in this section.

A non-limiting example of a mono-nucleotide substance of [Formula 1-A] is 7-deaza-8-aza-guanosine-3′-monophosphate wherein B is a nucleobase of [Formula 2-F], where V is C and U is N, R_{P1} is oxo, R_{P2} is amino, R_{P3} is H, and R_{P4} is absent, X_{D1} is H, and X_{D2} is absent, and Y₁ and Y₃ are hydroxy, and Y₂ is phosphate.

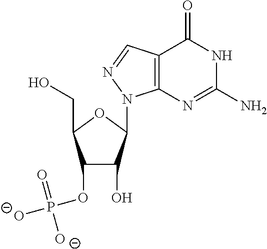

Other non-limiting examples of mono-nucleoside or mono-nucleotide substances may include the following:

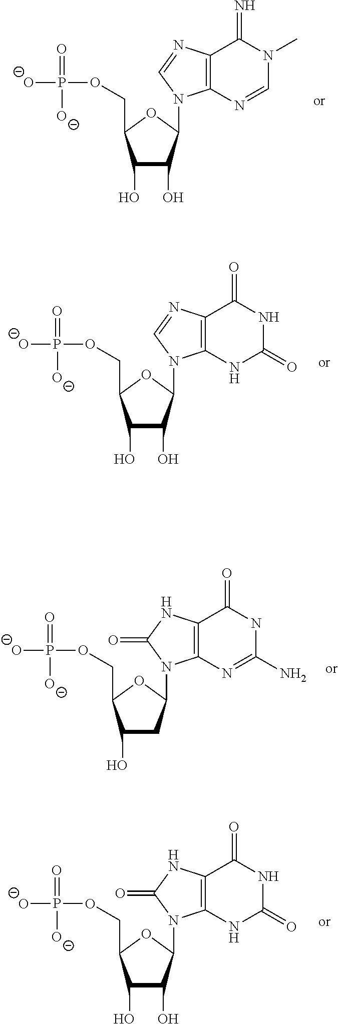

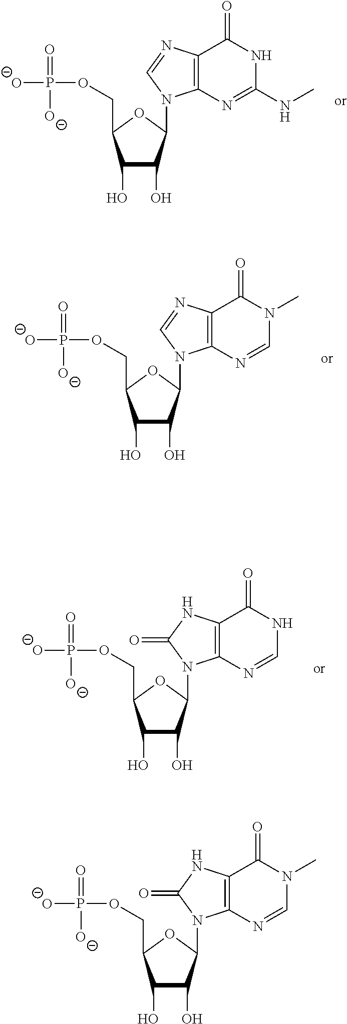

In some embodiments an RNA stabilizing substance may comprise a mono-nucleotide substance that has the formula [Formula 1-B]:

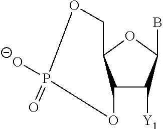

wherein:
B is a nucleobase described in this section;
Y₁ is an independent Y group described in this section.

A non-limiting example of a mono-nucleotide substance of [Formula 1-B] is guanosine-3′,5′-cyclic-monophosphate (also known as cyclic guanosine monophosphate, cyclic-GMP, or cGMP) wherein B is the nucleobase guanine and Y₁ is hydroxy.

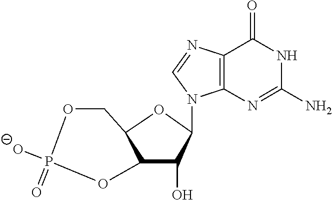

In some embodiments an RNA stabilizing substance may comprise a mono-nucleotide substance that has the formula [Formula 1-C]:

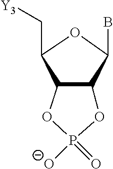

wherein:
B is a nucleobase described in this section;
Y₃ is an independent Y group described in this section.

A non-limiting example of a mono-nucleotide substance of [Formula 1-C] is uridine-2′,3′-cyclic monophosphate (also known as 2′,3′ cyclic UMP) wherein B is the nucleobase uracil and Y₃ is hydroxy.

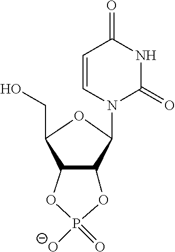

In some embodiments a mono-nucleoside substance or mono-nucleotide substance may comprise a purine nucleobase. In some embodiments a mono-nucleoside substance or mono-nucleotide substance may comprise a purine nucleobase wherein the purine nucleobase may be an unmodified or modified nucleobase. In some embodiments a mono-nucleoside substance or mono-nucleotide substance may comprise a purine nucleobase wherein the purine nucleobase may be a nucleobase analog.

In some embodiments a mono-nucleoside substance or mono-nucleotide substance may comprise a guanine nucleobase or isoguanine nucleobase, wherein the nucleobase may be a modified or unmodified nucleobase.

As used herein, an isoguanine nucleobase comprises an oxo at position 2 and an amino at position 6 of the purine ring (as a non-limiting example, isoguanine may also be known as 2-oxoadenine).

A mono-nucleoside or mono-nucleotide comprising a guanine nucleobase is herein referred to as a guanosine-mono-nucleoside or guanosine-mono-nucleotide. A mono-nucleoside or mono-nucleotide comprising an isoguanine nucleobase is herein referred to as an isoguanosine-mono-nucleoside or isoguanosine-mono-nucleotide.

In some embodiments a mono-nucleoside substance or mono-nucleotide substance may comprise a modified or unmodified guanosine-mono-nucleoside or a modified or unmodified guanosine-mono-nucleotide; or a modified or unmodified isoguanosine-mono-nucleoside or a modified or unmodified isoguanosine-mono-nucleotide.

In some embodiments non-limiting examples of a modified or unmodified guanine nucleobase or a modified or unmodified isoguanine nucleobase may include: guanine; N1-methyl-guanine; N2-methyl-guanine; N2,N2-dimethyl-guanine; N2-acetyl-guanine; N3-methyl-guanine; N7-methyl-guanine; 8-oxo-guanine; 8-hydroxy-guanine; N1,N2-dimethyl-guanine; N1,N2,N2-trimethyl-guanine; N1,N3-dimethyl-guanine; N2,N3-dimethyl-guanine; N2,N2,N3-trimethyl-guanine; N1,N2,N3-trimethyl-guanine; N1,N2,N2,N3-tetramethyl-guanine; isoguanine; N6-methyl-isoguanine; N6,N6-dimethyl-isoguanine; N6-acetyl-isoguanine; N1-methyl-isoguanine; N3-methyl-isoguanine; N7-methyl-isoguanine; 8-oxo-isoguanine; 8-hydroxy-isoguanine; N1,N6-dimethyl-isoguanine; N1,N6,N6-trimethyl-isoguanine; N1,N7-dimethyl-isoguanine; N2,N7-dimethyl-isoguanine; N2,N2,N7-trimethyl-isoguanine; N1,N2,N7-trimethyl-isoguanine; or N1,N2,N2,N7-tetramethyl-isoguanine.

In some embodiments non-limiting examples of a modified or unmodified guanosine-mono-nucleoside or a modified or unmodified isoguanosine-mono-nucleoside may include: guanosine; N1-methyl-guanosine; N2-methyl-guanosine; N2,N2-dimethyl-guanosine; N2-acetyl-guanosine; N3-methyl-guanosine; N7-methyl-guanosine; 8-oxo-guanosine; 8-hydroxy-guanosine; N1,N2-dimethyl-guanosine; N1,N2,N2-trimethyl-guanosine; N1,N3-dimethyl-guanosine; N2,N3-dimethyl-guanosine; N2,N2,N3-trimethyl-guanosine; N1,N2,N3-trimethyl-guanosine; N1,N2,N2,N3-tetramethyl-guanosine; 2′-deoxyguanosine; N1-methyl-2′-deoxyguanosine; N2-methyl-2′-deoxyguanosine; N2,N2-dimethyl-2′-deoxyguanosine; N2-acetyl-2′-deoxyguanosine; N3-methyl-2′-deoxyguanosine; N7-methyl-2′-deoxyguanosine; 8-oxo-2′-deoxyguanosine; 8-hydroxy-2′-deoxyguanosine; N1,N2-dimethyl-2′-deoxyguanosine; N1,N2,N2-trimethyl-2′-deoxyguanosine; N1,N3-dimethyl-2′-deoxyguanosine; N2,N3-dimethyl-2′-deoxyguanosine; N2,N2,N3-trimethyl-2′-deoxyguanosine; N1,N2,N3-trimethyl-2′-deoxyguanosine; N1,N2,N2,N3-tetramethyl-2′-deoxyguanosine; 2′-O-methylguanosine; N1-methyl-2′-O-methylguanosine; N2-methyl-2′-O-methylguanosine; N2,N2-dimethyl-2′-O-methylguanosine; N2-acetyl-2′-O-methylguanosine; N3-methyl-2′-O-methylguanosine; N7-methyl-2′-O-methylguanosine; 8-oxo-2′-O-methylguanosine; 8-hydroxy-2′-O-methylguanosine; N1,N2-dimethyl-2′-O-methylguanosine; N1,N2,N2-trimethyl-2′-O-methylguanosine; N1,N3-dimethyl-2′-O-methylguanosine; N2,N3-dimethyl-2′-O-methylguanosine; N2,N2,N3-trimethyl-2′-O-methylguanosine; N1,N2,N3-trimethyl-2′-O-methylguanosine; N1,N2,N2,N3-tetramethyl-2′-O-methylguanosine; 2′,3′,5′-tri-O-acetylguanosine; N1-methyl-2′,3′,5′-tri-O-acetylguanosine; N2-methyl-2′,3′,5′-tri-O-acetylguanosine; N2,N2-dimethyl-2′,3′,5′-tri-O-acetylguanosine; N2-acetyl-2′,3′,5′-tri-O-acetylguanosine; N3-methyl-2′,3′,5′-tri-O-acetylguanosine; N7-methyl-2′,3′,5′-tri-O-acetylguanosine; 8-oxo-2′,3′,5′-tri-O-acetylguanosine; 8-hydroxy-2′,3′,5′-tri-O-acetylguanosine; N1,N2-dimethyl-2′,3′,5′-tri-O-acetylguanosine; N1,N2,N2-trimethyl-2′,3′,5′-tri-O-acetylguanosine; N1,N3-dimethyl-2′,3′,5′-tri-O-acetylguanosine; N2,N3-dimethyl-2′,3′,5′-tri-O-acetylguanosine; N2,N2,N3-trimethyl-2′,3′,5′-tri-O-acetylguanosine; N1,N2,N3-trimethyl-2′,3′,5′-tri-O-acetylguanosine; N1,N2,N2,N3-tetramethyl-2′,3′,5′-tri-O-acetylguanosine; isoguanosine; N6-methyl-isoguanosine; N6,N6-dimethyl-isoguanosine; N6-acetyl-isoguanosine; N1-methyl-isoguanosine; N3-methyl-isoguanosine; N7-methyl-isoguanosine; 8-oxo-isoguanosine; 8-hydroxy-isoguanosine; N1,N6-dimethyl-isoguanosine; N1,N6,N6-trimethyl-isoguanosine; N1,N7-dimethyl-isoguanosine; N2,N7-dimethyl-isoguanosine; N2,N2,N7-trimethyl-isoguanosine; N1,N2,N7-trimethyl-isoguanosine; N1,N2,N2,N7-tetramethyl-isoguanosine; 2′-deoxy-isoguanosine; N6-methyl-2′-deoxy-isoguanosine; N6,N6-dimethyl-2′-deoxy-isoguanosine; N6-acetyl-2′-deoxy-isoguanosine; N1-methyl-2′-deoxy-isoguanosine; N3-methyl-2′-deoxy-isoguanosine; N7-methyl-2′-deoxy-isoguanosine; 8-oxo-2′-deoxy-isoguanosine; 8-hydroxy-2′-deoxy-isoguanosine; N1,N6-dimethyl-2′-deoxy-isoguanosine; N1,N6,N6-trimethyl-2′-deoxy-isoguanosine; N1,N7-dimethyl-2′-deoxy-isoguanosine; N2,N7-dimethyl-2′-deoxy-isoguanosine; N2,N2,N7-trimethyl-2′-deoxy-isoguanosine; N1,N2,N7-trimethyl-2′-deoxy-isoguanosine; N1,N2,N2,N7-tetramethyl-2′-deoxy-isoguanosine; 2′-O-methyl-isoguanosine; N6-methyl-2′-O-methyl-isoguanosine; N6,N6-dimethyl-2′-O-methyl-isoguanosine; N6-acetyl-2′-O-methyl-isoguanosine; N1-methyl-2′-O-methyl-isoguanosine; N3-methyl-2′-O-methyl-isoguanosine; N7-methyl-2′-O-methyl-isoguanosine; 8-oxo-2′-O-methyl-isoguanosine; 8-hydroxy-2′-O-methyl-isoguanosine; N1,N6-dimethyl-2′-O-methyl-isoguanosine; N1,N6,N6-trimethyl-2′-O-methyl-isoguanosine; N1,N7-dimethyl-2′-O-methyl-isoguanosine; N2,N7-dimethyl-2′-O-methyl-isoguanosine; N2,N2,N7-trimethyl-2′-O-methyl-isoguanosine; N1,N2,N7-trimethyl-2′-O-methyl-isoguanosine; N1,N2,N2,N7-tetramethyl-2′-O-methyl-isoguanosine; 2′,3′,5′-tri-O-acetyl-isoguanosine; N6-methyl-2′,3′,5′-tri-O-acetyl-isoguanosine; N6,N6-dimethyl-2′,3′,5′-tri-O-acetyl-isoguanosine; N6-acetyl-2′,3′,5′-tri-O-acetyl-isoguanosine; N1-methyl-2′,3′,5′-tri-O-acetyl-isoguanosine; N3-methyl-2′,3′,5′-tri-O-acetyl-isoguanosine; N7-methyl-2′,3′,5′-tri-O-acetyl-isoguanosine; 8-oxo-2′,3′,5′-tri-O-acetyl-isoguanosine; 8-hydroxy-2′,3′,5′-tri-O-acetyl-isoguanosine; N1,N6-dimethyl-2′,3′,5′-tri-O-acetyl-isoguanosine; N1,N6,N6-trimethyl-2′,3′,5′-tri-O-acetyl-isoguanosine; N1,N7-dimethyl-2′,3′,5′-tri-O-acetyl-isoguanosine; N2,N7-dimethyl-2′,3′,5′-tri-O-acetyl-isoguanosine; N2,N2,N7-trimethyl-2′,3′,5′-tri-O-acetyl-isoguanosine; N1,N2,N7-trimethyl-2′,3′,5′-tri-O-acetyl-isoguanosine; or N1,N2,N2,N7-tetramethyl-2′,3′,5′-tri-O-acetyl-isoguanosine.

In some embodiments non-limiting examples of a modified or unmodified guanosine-mono-nucleotide or a modified or unmodified isoguanosine-mono-nucleotide may include: guanosine-5′-monophosphate; N1-methyl-guanosine-5′-monophosphate; N2-methyl-guanosine-5′-monophosphate; N2,N2-dimethyl-guanosine-5′-monophosphate; N2-acetyl-guanosine-5′-monophosphate; N3-methyl-guanosine-5′-monophosphate; N7-methyl-guanosine-5′-monophosphate; 8-oxo-guanosine-5′-monophosphate; 8-hydroxy-guanosine-5′-monophosphate; N1,N2-dimethyl-guanosine-5′-monophosphate; N1,N2,N2-trimethyl-guanosine-5′-monophosphate; N1,N3-dimethyl-guanosine-5′-monophosphate; N2,N3-dimethyl-guanosine-5′-monophosphate; N2,N2,N3-trimethyl-guanosine-5′-monophosphate; N1,N2,N3-trimethyl-guanosine-5′-monophosphate; N1,N2,N2,N3-tetramethyl-guanosine-5′-monophosphate; guanosine-3′-monophosphate; N1-methyl-guanosine-3′-monophosphate; N2-methyl-guanosine-3′-monophosphate; N2,N2-dimethyl-guanosine-3′-monophosphate; N2-acetyl-guanosine-3′-monophosphate; N3-methyl-guanosine-3′-monophosphate; N7-methyl-guanosine-3′-monophosphate; 8-oxo-guanosine-3′-monophosphate; 8-hydroxy-guanosine-3′-monophosphate; N1,N2-dimethyl-guanosine-3′-monophosphate; N1,N2,N2-trimethyl-guanosine-3′-monophosphate; N1,N3-dimethyl-guanosine-3′-monophosphate; N2,N3-dimethyl-guanosine-3′-monophosphate; N2,N2,N3-trimethyl-guanosine-3′-monophosphate; N1,N2,N3-trimethyl-guanosine-3′-monophosphate; N1,N2,N2,N3-tetramethyl-guanosine-3′-monophosphate; 2′-deoxyguanosine-5′-monophosphate; N1-methyl-2′-deoxyguanosine-5′-monophosphate; N2-methyl-2′-deoxyguanosine-5′-monophosphate; N2,N2-dimethyl-2′-deoxyguanosine-5′-monophosphate; N2-acetyl-2′-deoxyguanosine-5′-monophosphate; N3-methyl-2′-deoxyguanosine-5′-monophosphate; N7-methyl-2′-deoxyguanosine-5′-monophosphate; 8-oxo-2′-deoxyguanosine-5′-monophosphate; 8-hydroxy-2′-deoxyguanosine-5′-monophosphate; N1,N2-dimethyl-2′-deoxyguanosine-5′-monophosphate; N1,N2,N2-trimethyl-2′-deoxyguanosine-5′-monophosphate; N1,N3-dimethyl-2′-deoxyguanosine-5′-monophosphate; N2,N3-dimethyl-2′-deoxyguanosine-5′-monophosphate; N2,N2,N3-trimethyl-2′-deoxyguanosine-5′-monophosphate; N1,N2,N3-trimethyl-2′-deoxyguanosine-5′-monophosphate; N1,N2,N2,N3-tetramethyl-2′-deoxyguanosine-5′-monophosphate; 2′-deoxyguanosine-3′-monophosphate; N1-methyl-2′-deoxyguanosine-3′-monophosphate; N2-methyl-2′-deoxyguanosine-3′-monophosphate; N2,N2-dimethyl-2′-deoxyguanosine-3′-monophosphate; N2-acetyl-2′-deoxyguanosine-3′-monophosphate; N3-methyl-2′-deoxyguanosine-3′-monophosphate; N7-methyl-2′-deoxyguanosine-3′-monophosphate; 8-oxo-2′-deoxyguanosine-3′-monophosphate; 8-hydroxy-2′-deoxyguanosine-3′-monophosphate; N1,N2-dimethyl-2′-deoxyguanosine-3′-monophosphate; N1,N2,N2-trimethyl-2′-deoxyguanosine-3′-monophosphate; N1,N3-dimethyl-2′-deoxyguanosine-3′-monophosphate; N2,N3-dimethyl-2′-deoxyguanosine-3′-monophosphate; N2,N2,N3-trimethyl-2′-deoxyguanosine-3′-monophosphate; N1,N2,N3-trimethyl-2′-deoxyguanosine-3′-monophosphate; N1,N2,N2,N3-tetramethyl-2′-deoxyguanosine-3′-monophosphate; isoguanosine-5′-monophosphate; N6-methyl-isoguanosine-5′-monophosphate; N6,N6-dimethyl-isoguanosine-5′-monophosphate; N6-acetyl-isoguanosine-5′-monophosphate; N1-methyl-isoguanosine-5′-monophosphate; N3-methyl-isoguanosine-5′-monophosphate; N7-methyl-isoguanosine-5′-monophosphate; 8-oxo-isoguanosine-5′-monophosphate; 8-hydroxy-isoguanosine-5′-monophosphate; N1,N6-dimethyl-isoguanosine-5′-monophosphate; N1,N6,N6-trimethyl-isoguanosine-5′-monophosphate; N1,N7-dimethyl-isoguanosine-5′-monophosphate; N2,N7-dimethyl-isoguanosine-5′-monophosphate; N2,N2,N7-trimethyl-isoguanosine-5′-monophosphate; N1,N2,N7-trimethyl-isoguanosine-5′-monophosphate; N1,N2,N2,N7-tetramethyl-isoguanosine-5′-monophosphate; isoguanosine-3′-monophosphate; N6-methyl-isoguanosine-3′-monophosphate; N6,N6-dimethyl-isoguanosine-3′-monophosphate; N6-acetyl-isoguanosine-3′-monophosphate; N1-methyl-isoguanosine-3′-monophosphate; N3-methyl-isoguanosine-3′-monophosphate; N7-methyl-isoguanosine-3′-monophosphate; 8-oxo-isoguanosine-3′-monophosphate; 8-hydroxy-isoguanosine-3′-monophosphate; N1,N6-dimethyl-isoguanosine-3′-monophosphate; N1,N6,N6-trimethyl-isoguanosine-3′-monophosphate; N1,N7-dimethyl-isoguanosine-3′-monophosphate; N2,N7-dimethyl-isoguanosine-3′-monophosphate; N2,N2,N7-trimethyl-isoguanosine-3′-monophosphate; N1,N2,N7-trimethyl-isoguanosine-3′-monophosphate; N1,N2,N2,N7-tetramethyl-isoguanosine-3′-monophosphate; 2′-deoxy-isoguanosine-5′-monophosphate; N6-methyl-2′-deoxy-isoguanosine-5′-monophosphate; N6,N6-dimethyl-2′-deoxy-isoguanosine-5′-monophosphate; N6-acetyl-2′-deoxy-isoguanosine-5′-monophosphate; N1-methyl-2′-deoxy-isoguanosine-5′-monophosphate; N3-methyl-2′-deoxy-isoguanosine-5′-monophosphate; N7-methyl-2′-deoxy-isoguanosine-5′-monophosphate; 8-oxo-2′-deoxy-isoguanosine-5′-monophosphate; 8-hydroxy-2′-deoxy-isoguanosine-5′-monophosphate; N1,N6-dimethyl-2′-deoxy-isoguanosine-5′-monophosphate; N1,N6,N6-trimethyl-2′-deoxy-isoguanosine-5′-monophosphate; N1,N7-dimethyl-2′-deoxy-isoguanosine-5′-monophosphate; N2,N7-dimethyl-2′-deoxy-isoguanosine-5′-monophosphate; N2,N2,N7-trimethyl-2′-deoxy-isoguanosine-5′-monophosphate; N1,N2,N7-trimethyl-2′-deoxy-isoguanosine-5′-monophosphate; N1,N2,N2,N7-tetramethyl-2′-deoxy-isoguanosine-5′-monophosphate; 2′-deoxy-isoguanosine-3′-monophosphate; N6-methyl-2′-deoxy-isoguanosine-3′-monophosphate; N6,N6-dimethyl-2′-deoxy-isoguanosine-3′-monophosphate; N6-acetyl-2′-deoxy-isoguanosine-3′-monophosphate; N1-methyl-2′-deoxy-isoguanosine-3′-monophosphate; N3-methyl-2′-deoxy-isoguanosine-3′-monophosphate; N7-methyl-2′-deoxy-isoguanosine-3′-monophosphate; 8-oxo-2′-deoxy-isoguanosine-3′-monophosphate; 8-hydroxy-2′-deoxy-isoguanosine-3′-monophosphate; N1,N6-dimethyl-2′-deoxy-isoguanosine-3′-monophosphate; N1,N6,N6-trimethyl-2′-deoxy-isoguanosine-3′-monophosphate; N1,N7-dimethyl-2′-deoxy-isoguanosine-3′-monophosphate; N2,N7-dimethyl-2′-deoxy-isoguanosine-3′-monophosphate; N2,N2,N7-trimethyl-2′-deoxy-isoguanosine-3′-monophosphate; N1,N2,N7-trimethyl-2′-deoxy-isoguanosine-3′-monophosphate; or N1,N2,N2,N7-tetramethyl-2′-deoxy-isoguanosine-3′-monophosphate.

In some embodiments a mono-nucleoside substance or mono-nucleotide substance may comprise an adenine nucleobase, wherein the nucleobase may be a modified or unmodified nucleobase.

A mono-nucleoside or mono-nucleotide comprising an adenine nucleobase is herein referred to as an adenosine-mono-nucleoside or adenosine-mono-nucleotide.

In some embodiments a mono-nucleoside substance or mono-nucleotide substance may comprise a modified or unmodified adenosine-mono-nucleoside or a modified or unmodified adenosine-mono-nucleotide.

In some embodiments non-limiting examples of a modified or unmodified adenine nucleobase may include: adenine; N6-methyl-adenine; N6,N6-dimethyl-adenine; N6-acetyl-adenine; N1-methyl-adenine; N3-methyl-adenine; N7-methyl-adenine; 8-oxo-adenine; 8-hydroxy-adenine; N1,N6-dimethyl-adenine; N1,N6,N6-trimethyl-adenine; N1,N3-dimethyl-adenine; N1,N3,N6-trimethyl-adenine; N1,N3,N6,N6-tetramethyl-adenine; N1-methyl-8-oxo-adenine; or N6,N6,-dimethyl-8-oxo-adenine.

In some embodiments non-limiting examples of a modified or unmodified adenosine-mono-nucleoside may include: adenosine; N6-methyl-adenosine; N6,N6-dimethyl-adenosine; N6-acetyl-adenosine; N1-methyl-adenosine; N3-methyl-adenosine; N7-methyl-adenosine; 8-oxo-adenosine; 8-hydroxy-adenosine; N1,N6-dimethyl-adenosine; N1,N6,N6-trimethyl-adenosine; N1,N3-dimethyl-adenosine; N1,N3,N6-trimethyl-adenosine; N1,N3,N6,N6-tetramethyl-adenosine; N1-methyl-8-oxo-adenosine; N6,N6,-dimethyl-8-oxo-adenosine; 2′-deoxyadenosine; N6-methyl-2′-deoxyadenosine; N6,N6-dimethyl-2′-deoxyadenosine; N6-acetyl-2′-deoxyadenosine; N1-methyl-2′-deoxyadenosine; N3-methyl-2′-deoxyadenosine; N7-methyl-2′-deoxyadenosine; 8-oxo-2′-deoxyadenosine; 8-hydroxy-2′-deoxyadenosine; N1,N6-dimethyl-2′-deoxyadenosine; N1,N6,N6-trimethyl-2′-deoxyadenosine; N1,N3-dimethyl-2′-deoxyadenosine; N1,N3,N6-trimethyl-2′-deoxyadenosine; N1,N3,N6,N6-tetramethyl-2′-deoxyadenosine; N1-methyl-8-oxo-2′-deoxyadenosine; N6,N6,-dimethyl-8-oxo-2′-deoxyadenosine; 2′-O-methyladenosine; N6-methyl-2′-O-methyladenosine; N6,N6-dimethyl-2′-O-methyladenosine; N6-acetyl-2′-O-methyladenosine; N1-methyl-2′-O-methyladenosine; N3-methyl-2′-O-methyladenosine; N7-methyl-2′-O-methyladenosine; 8-oxo-2′-O-methyladenosine; 8-hydroxy-2′-O-methyladenosine; N1,N6-dimethyl-2′-O-methyladenosine; N1,N6,N6-trimethyl-2′-O-methyladenosine; N1,N3-dimethyl-2′-O-methyladenosine; N1,N3,N6-trimethyl-2′-O-methyladenosine; N1,N3,N6,N6-tetramethyl-2′-O-methyladenosine; N1-methyl-8-oxo-2′-O-methyladenosine; N6,N6,-dimethyl-8-oxo-2′-O-methyladenosine; 2′,3′,5′-tri-O-acetyladenosine; N6-methyl-2′,3′,5′-tri-O-acetyladenosine; N6,N6-dimethyl-2′,3′,5′-tri-O-acetyladenosine; N6-acetyl-2′,3′,5′-tri-O-acetyladenosine; N1-methyl-2′,3′,5′-tri-O-acetyladenosine; N3-methyl-2′,3′,5′-tri-O-acetyladenosine; N7-methyl-2′,3′,5′-tri-O-acetyladenosine; 8-oxo-2′,3′,5′-tri-O-acetyladenosine; 8-hydroxy-2′,3′,5′-tri-O-acetyladenosine; N1,N6-dimethyl-2′,3′,5′-tri-O-acetyladenosine; N1,N6,N6-trimethyl-2′,3′,5′-tri-O-acetyladenosine; N1,N3-dimethyl-2′,3′,5′-tri-O-acetyladenosine; N1,N3,N6-trimethyl-2′,3′,5′-tri-O-acetyladenosine; N1,N3,N6,N6-tetramethyl-2′,3′,5′-tri-O-acetyladenosine; N1-methyl-8-oxo-2′,3′,5′-tri-O-acetyladenosine; or N6,N6,-dimethyl-8-oxo-2′,3′,5′-tri-O-acetyladenosine.

In some embodiments non-limiting examples of a modified or unmodified adenosine-mono-nucleotide may include: adenosine-5′-monophosphate; N6-methyl-adenosine-5′-monophosphate; N6,N6-dimethyl-adenosine-5′-monophosphate; N6-acetyl-adenosine-5′-monophosphate; N1-methyl-adenosine-5′-monophosphate; N3-methyl-adenosine-5′-monophosphate; N7-methyl-adenosine-5′-monophosphate; 8-oxo-adenosine-5′-monophosphate; 8-hydroxy-adenosine-5′-monophosphate; N1,N6-dimethyl-adenosine-5′-monophosphate; N1,N6,N6-trimethyl-adenosine-5′-monophosphate; N1,N3-dimethyl-adenosine-5′-monophosphate; N1,N3,N6-trimethyl-adenosine-5′-monophosphate; N1,N3,N6,N6-tetramethyl-adenosine-5′-monophosphate; N1-methyl-8-oxo-adenosine-5′-monophosphate; N6,N6,-dimethyl-8-oxo-adenosine-5′-monophosphate; adenosine-3′-monophosphate; N6-methyl-adenosine-3′-monophosphate; N6,N6-dimethyl-adenosine-3′-monophosphate; N6-acetyl-adenosine-3′-monophosphate; N1-methyl-adenosine-3′-monophosphate; N3-methyl-adenosine-3′-monophosphate; N7-methyl-adenosine-3′-monophosphate; 8-oxo-adenosine-3′-monophosphate; 8-hydroxy-adenosine-3′-monophosphate; N1,N6-dimethyl-adenosine-3′-monophosphate; N1,N6,N6-trimethyl-adenosine-3′-monophosphate; N1,N3-dimethyl-adenosine-3′-monophosphate; N1,N3,N6-trimethyl-adenosine-3′-monophosphate; N1,N3,N6,N6-tetramethyl-adenosine-3′-monophosphate; N1-methyl-8-oxo-adenosine-3′-monophosphate; N6,N6,-dimethyl-8-oxo-adenosine-3′-monophosphate; 2′-deoxyadenosine-5′-monophosphate; N6-methyl-2′-deoxyadenosine-5′-monophosphate; N6,N6-dimethyl-2′-deoxyadenosine-5′-monophosphate; N6-acetyl-2′-deoxyadenosine-5′-monophosphate; N1-methyl-2′-deoxyadenosine-5′-monophosphate; N3-methyl-2′-deoxyadenosine-5′-monophosphate; N7-methyl-2′-deoxyadenosine-5′-monophosphate; 8-oxo-2′-deoxyadenosine-5′-monophosphate; 8-hydroxy-2′-deoxyadenosine-5′-monophosphate; N1,N6-dimethyl-2′-deoxyadenosine-5′-monophosphate; N1,N6,N6-trimethyl-2′-deoxyadenosine-5′-monophosphate; N1,N3-dimethyl-2′-deoxyadenosine-5′-monophosphate; N1,N3,N6-trimethyl-2′-deoxyadenosine-5′-monophosphate; N1,N3,N6,N6-tetramethyl-2′-deoxyadenosine-5′-monophosphate; N1-methyl-8-oxo-2′-deoxyadenosine-5′-monophosphate; N6,N6,-dimethyl-8-oxo-2′-deoxyadenosine-5′-monophosphate; 2′-deoxyadenosine-3′-monophosphate; N6-methyl-2′-deoxyadenosine-3′-monophosphate; N6,N6-dimethyl-2′-deoxyadenosine-3′-monophosphate; N6-acetyl-2′-deoxyadenosine-3′-monophosphate; N1-methyl-2′-deoxyadenosine-3′-monophosphate; N3-methyl-2′-deoxyadenosine-3′-monophosphate; N7-methyl-2′-deoxyadenosine-3′-monophosphate; 8-oxo-2′-deoxyadenosine-3′-monophosphate; 8-hydroxy-2′-deoxyadenosine-3′-monophosphate; N1,N6-dimethyl-2′-deoxyadenosine-3′-monophosphate; N1,N6,N6-trimethyl-2′-deoxyadenosine-3′-monophosphate; N1,N3-dimethyl-2′-deoxyadenosine-3′-monophosphate; N1,N3,N6-trimethyl-2′-deoxyadenosine-3′-monophosphate; N1,N3,N6,N6-tetramethyl-2′-deoxyadenosine-3′-monophosphate; N1-methyl-8-oxo-2′-deoxyadenosine-3′-monophosphate; or N6,N6,-dimethyl-8-oxo-2′-deoxyadenosine-3′-monophosphate.

In some embodiments a nucleobase may comprise hypoxanthine (also known as 6-hydroxypurine or 6-oxopurine, depending on the tautomer) or isohypoxanthine (also known as 2-hydroxypurine or 2-oxopurine, depending on the tautomer).

In some embodiments a mono-nucleoside substance or mono-nucleotide substance may comprise a hypoxanthine nucleobase or isohypoxanthine nucleobase, wherein the nucleobase may be a modified or unmodified nucleobase.

A mono-nucleoside or mono-nucleotide comprising a hypoxanthine nucleobase is herein referred to as an inosine-mono-nucleoside or inosine-mono-nucleotide. A mono-nucleoside or mono-nucleotide comprising an isohypoxanthine nucleobase is herein referred to as an isoinosine-mono-nucleoside or isoinosine-mono-nucleotide.

In some embodiments a mono-nucleoside substance or mono-nucleotide substance may comprise a modified or unmodified inosine-mono-nucleoside or a modified or unmodified inosine-mono-nucleotide; or a modified or unmodified isoinosine-mono-nucleoside or a modified or unmodified isoinosine-mono-nucleotide.

In some embodiments non-limiting examples of a modified or unmodified hypoxanthine nucleobase or modified or unmodified isohypoxanthine nucleobase may include: hypoxanthine; N1-methyl-hypoxanthine; N3-methyl-hypoxanthine; N7-methyl-hypoxanthine; 8-oxo-hypoxanthine; 8-hydroxy-hypoxanthine; N1,N3-dimethyl-hypoxanthine; N3,N7-dimethyl-hypoxanthine; N1,N7-dimethyl-hypoxanthine; N1,N3,N7-trimethyl-hypoxanthine; N1-methyl-8-oxo-hypoxanthine; N3-methyl-8-oxo-hypoxanthine; N1,N3-dimethyl-8-oxo-hypoxanthine; isohypoxanthine; N1-methyl-isohypoxanthine; N3-methyl-isohypoxanthine; N7-methyl-isohypoxanthine; 8-oxo-isohypoxanthine; 8-hydroxy-isohypoxanthine; N1,N3-dimethyl-isohypoxanthine; N3,N7-dimethyl-isohypoxanthine; N1,N7-dimethyl-isohypoxanthine; N1,N3,N7-trimethyl-isohypoxanthine; N1-methyl-8-oxo-isohypoxanthine; N3-methyl-8-oxo-isohypoxanthine; or N1,N3-dimethyl-8-oxo-isohypoxanthine.

In some embodiments non-limiting examples of a modified or unmodified inosine-mono-nucleoside or a modified or unmodified isoinosine-mono-nucleoside may include: inosine; N1-methyl-inosine; N3-methyl-inosine; N7-methyl-inosine; 8-oxo-inosine; 8-hydroxy-inosine; N1,N3-dimethyl-inosine; N3,N7-dimethyl-inosine; N1,N7-dimethyl-inosine; N1,N3,N7-trimethyl-inosine; N1-methyl-8-oxo-inosine; N3-methyl-8-oxo-inosine; N1,N3-dimethyl-8-oxo-inosine; 2′-deoxyinosine; N1-methyl-2′-deoxyinosine; N3-methyl-2′-deoxyinosine; N7-methyl-2′-deoxyinosine; 8-oxo-2′-deoxyinosine; 8-hydroxy-2′-deoxyinosine; N1,N3-dimethyl-2′-deoxyinosine; N3,N7-dimethyl-2′-deoxyinosine; N1,N7-dimethyl-2′-deoxyinosine; N1,N3,N7-trimethyl-2′-deoxyinosine; N1-methyl-8-oxo-2′-deoxyinosine; N3-methyl-8-oxo-2′-deoxyinosine; N1,N3-dimethyl-8-oxo-2′-deoxyinosine; 2′-O-methylinosine; N1-methyl-2′-O-methylinosine; N3-methyl-2′-O-methylinosine; N7-methyl-2′-O-methylinosine; 8-oxo-2′-O-methylinosine; 8-hydroxy-2′-O-methylinosine; N1,N3-dimethyl-2′-O-methylinosine; N3,N7-dimethyl-2′-O-methylinosine; N1,N7-dimethyl-2′-O-methylinosine; N1,N3,N7-trimethyl-2′-O-methylinosine; N1-methyl-8-oxo-2′-O-methylinosine; N3-methyl-8-oxo-2′-O-methylinosine; N1,N3-dimethyl-8-oxo-2′-O-methylinosine; 2′,3′,5′-tri-O-acetylinosine; N1-methyl-2′,3′,5′-tri-O-acetylinosine; N3-methyl-2′,3′,5′-tri-O-acetylinosine; N7-methyl-2′,3′,5′-tri-O-acetylinosine; 8-oxo-2′,3′,5′-tri-O-acetylinosine; 8-hydroxy-2′,3′,5′-tri-O-acetylinosine; N1,N3-dimethyl-2′,3′,5′-tri-O-acetylinosine; N3,N7-dimethyl-2′,3′,5′-tri-O-acetylinosine; N1,N7-dimethyl-2′,3′,5′-tri-O-acetylinosine; N1,N3,N7-trimethyl-2′,3′,5′-tri-O-acetylinosine; N1-methyl-8-oxo-2′,3′,5′-tri-O-acetylinosine; N3-methyl-8-oxo-2′,3′,5′-tri-O-acetylinosine; N1,N3-dimethyl-8-oxo-2′,3′,5′-tri-O-acetylinosine; isoinosine; N1-methyl-isoinosine; N3-methyl-isoinosine; N7-methyl-isoinosine; 8-oxo-isoinosine; 8-hydroxy-isoinosine; N1,N3-dimethyl-isoinosine; N3,N7-dimethyl-isoinosine; N1,N7-dimethyl-isoinosine; N1,N3,N7-trimethyl-isoinosine; N1-methyl-8-oxo-isoinosine; N3-methyl-8-oxo-isoinosine; N1,N3-dimethyl-8-oxo-isoinosine; 2′-deoxy-isoinosine; N1-methyl-2′-deoxy-isoinosine; N3-methyl-2′-deoxy-isoinosine; N7-methyl-2′-deoxy-isoinosine; 8-oxo-2′-deoxy-isoinosine; 8-hydroxy-2′-deoxy-isoinosine; N1,N3-dimethyl-2′-deoxy-isoinosine; N3,N7-dimethyl-2′-deoxy-isoinosine; N1,N7-dimethyl-2′-deoxy-isoinosine; N1,N3,N7-trimethyl-2′-deoxy-isoinosine; N1-methyl-8-oxo-2′-deoxy-isoinosine; N3-methyl-8-oxo-2′-deoxy-isoinosine; N1,N3-dimethyl-8-oxo-2′-deoxy-isoinosine; 2′-O-methyl-isoinosine; N1-methyl-2′-O-methyl-isoinosine; N3-methyl-2′-O-methyl-isoinosine; N7-methyl-2′-O-methyl-isoinosine; 8-oxo-2′-O-methyl-isoinosine; 8-hydroxy-2′-O-methyl-isoinosine; N1,N3-dimethyl-2′-O-methyl-isoinosine; N3,N7-dimethyl-2′-O-methyl-isoinosine; N1,N7-dimethyl-2′-O-methyl-isoinosine; N1,N3,N7-trimethyl-2′-O-methyl-isoinosine; N1-methyl-8-oxo-2′-O-methyl-isoinosine; N3-methyl-8-oxo-2′-O-methyl-isoinosine; N1,N3-dimethyl-8-oxo-2′-O-methyl-isoinosine; 2′,3′,5′-tri-O-acetyl-isoinosine; N1-methyl-2′,3′,5′-tri-O-acetyl-isoinosine; N3-methyl-2′,3′,5′-tri-O-acetyl-isoinosine; N7-methyl-2′,3′,5′-tri-O-acetyl-isoinosine; 8-oxo-2′,3′,5′-tri-O-acetyl-isoinosine; 8-hydroxy-2′,3′,5′-tri-O-acetyl-isoinosine; N1,N3-dimethyl-2′,3′,5′-tri-O-acetyl-isoinosine; N3,N7-dimethyl-2′,3′,5′-tri-O-acetyl-isoinosine; N1,N7-dimethyl-2′,3′,5′-tri-O-acetyl-isoinosine; N1,N3,N7-trimethyl-2′,3′,5′-tri-O-acetyl-isoinosine; N1-methyl-8-oxo-2′,3′,5′-tri-O-acetyl-isoinosine; N3-methyl-8-oxo-2′,3′,5′-tri-O-acetyl-isoinosine; or N1,N3-dimethyl-8-oxo-2′,3′,5′-tri-O-acetyl-isoinosine.

In some embodiments non-limiting examples of a modified or unmodified inosine-mono-nucleotide or a modified or unmodified isoinosine-mono-nucleotide may include: inosine-5′-monophosphate; N1-methyl-inosine-5′-monophosphate; N3-methyl-inosine-5′-monophosphate; N7-methyl-inosine-5′-monophosphate; 8-oxo-inosine-5′-monophosphate; 8-hydroxy-inosine-5′-monophosphate; N1,N3-dimethyl-inosine-5′-monophosphate; N3,N7-dimethyl-inosine-5′-monophosphate; N1,N7-dimethyl-inosine-5′-monophosphate; N1,N3,N7-trimethyl-inosine-5′-monophosphate; N1-methyl-8-oxo-inosine-5′-monophosphate; N3-methyl-8-oxo-inosine-5′-monophosphate; N1,N3-dimethyl-8-oxo-inosine-5′-monophosphate; inosine-3′-monophosphate; N1-methyl-inosine-3′-monophosphate; N3-methyl-inosine-3′-monophosphate; N7-methyl-inosine-3′-monophosphate; 8-oxo-inosine-3′-monophosphate; 8-hydroxy-inosine-3′-monophosphate; N1,N3-dimethyl-inosine-3′-monophosphate; N3,N7-dimethyl-inosine-3′-monophosphate; N1,N7-dimethyl-inosine-3′-monophosphate; N1,N3,N7-trimethyl-inosine-3′-monophosphate; N1-methyl-8-oxo-inosine-3′-monophosphate; N3-methyl-8-oxo-inosine-3′-monophosphate; N1,N3-dimethyl-8-oxo-inosine-3′-monophosphate; 2′-deoxyinosine-5′-monophosphate; N1-methyl-2′-deoxyinosine-5′-monophosphate; N3-methyl-2′-deoxyinosine-5′-monophosphate; N7-methyl-2′-deoxyinosine-5′-monophosphate; 8-oxo-2′-deoxyinosine-5′-monophosphate; 8-hydroxy-2′-deoxyinosine-5′-monophosphate; N1,N3-dimethyl-2′-deoxyinosine-5′-monophosphate; N3,N7-dimethyl-2′-deoxyinosine-5′-monophosphate; N1,N7-dimethyl-2′-deoxyinosine-5′-monophosphate; N1,N3,N7-trimethyl-2′-deoxyinosine-5′-monophosphate; N1-methyl-8-oxo-2′-deoxyinosine-5′-monophosphate; N3-methyl-8-oxo-2′-deoxyinosine-5′-monophosphate; N1,N3-dimethyl-8-oxo-2′-deoxyinosine-5′-monophosphate; 2′-deoxyinosine-3′-monophosphate; N1-methyl-2′-deoxyinosine-3′-monophosphate; N3-methyl-2′-deoxyinosine-3′-monophosphate; N7-methyl-2′-deoxyinosine-3′-monophosphate; 8-oxo-2′-deoxyinosine-3′-monophosphate; 8-hydroxy-2′-deoxyinosine-3′-monophosphate; N1,N3-dimethyl-2′-deoxyinosine-3′-monophosphate; N3,N7-dimethyl-2′-deoxyinosine-3′-monophosphate; N1,N7-dimethyl-2′-deoxyinosine-3′-monophosphate; N1,N3,N7-trimethyl-2′-deoxyinosine-3′-monophosphate; N1-methyl-8-oxo-2′-deoxyinosine-3′-monophosphate; N3-methyl-8-oxo-2′-deoxyinosine-3′-monophosphate; N1,N3-dimethyl-8-oxo-2′-deoxyinosine-3′-monophosphate; isoinosine-5′-monophosphate; N1-methyl-isoinosine-5′-monophosphate; N3-methyl-isoinosine-5′-monophosphate; N7-methyl-isoinosine-5′-monophosphate; 8-oxo-isoinosine-5′-monophosphate; 8-hydroxy-isoinosine-5′-monophosphate; N1,N3-dimethyl-isoinosine-5′-monophosphate; N3,N7-dimethyl-isoinosine-5′-monophosphate; N1,N7-dimethyl-isoinosine-5′-monophosphate; N1,N3,N7-trimethyl-isoinosine-5′-monophosphate; N1-methyl-8-oxo-isoinosine-5′-monophosphate; N3-methyl-8-oxo-isoinosine-5′-monophosphate; N1,N3-dimethyl-8-oxo-isoinosine-5′-monophosphate; isoinosine-3′-monophosphate; N1-methyl-isoinosine-3′-monophosphate; N3-methyl-isoinosine-3′-monophosphate; N7-methyl-isoinosine-3′-monophosphate; 8-oxo-isoinosine-3′-monophosphate; 8-hydroxy-isoinosine-3′-monophosphate; N1,N3-dimethyl-isoinosine-3′-monophosphate; N3,N7-dimethyl-isoinosine-3′-monophosphate; N1,N7-dimethyl-isoinosine-3′-monophosphate; N1,N3,N7-trimethyl-isoinosine-3′-monophosphate; N1-methyl-8-oxo-isoinosine-3′-monophosphate; N3-methyl-8-oxo-isoinosine-3′-monophosphate; N1,N3-dimethyl-8-oxo-isoinosine-3′-monophosphate; 2′-deoxy-isoinosine-5′-monophosphate; N1-methyl-2′-deoxy-isoinosine-5′-monophosphate; N3-methyl-2′-deoxy-isoinosine-5′-monophosphate; N7-methyl-2′-deoxy-isoinosine-5′-monophosphate; 8-oxo-2′-deoxy-isoinosine-5′-monophosphate; 8-hydroxy-2′-deoxy-isoinosine-5′-monophosphate; N1,N3-dimethyl-2′-deoxy-isoinosine-5′-monophosphate; N3,N7-dimethyl-2′-deoxy-isoinosine-5′-monophosphate; N1,N7-dimethyl-2′-deoxy-isoinosine-5′-monophosphate; N1,N3,N7-trimethyl-2′-deoxy-isoinosine-5′-monophosphate; N1-methyl-8-oxo-2′-deoxy-isoinosine-5′-monophosphate; N3-methyl-8-oxo-2′-deoxy-isoinosine-5′-monophosphate; N1,N3-dimethyl-8-oxo-2′-deoxy-isoinosine-5′-monophosphate; 2′-deoxy-isoinosine-3′-monophosphate; N1-methyl-2′-deoxy-isoinosine-3′-monophosphate; N3-methyl-2′-deoxy-isoinosine-3′-monophosphate; N7-methyl-2′-deoxy-isoinosine-3′-monophosphate; 8-oxo-2′-deoxy-isoinosine-3′-monophosphate; 8-hydroxy-2′-deoxy-isoinosine-3′-monophosphate; N1,N3-dimethyl-2′-deoxy-isoinosine-3′-monophosphate; N3,N7-dimethyl-2′-deoxy-isoinosine-3′-monophosphate; N1,N7-dimethyl-2′-deoxy-isoinosine-3′-monophosphate; N1,N3,N7-trimethyl-2′-deoxy-isoinosine-3′-monophosphate; N1-methyl-8-oxo-2′-deoxy-isoinosine-3′-monophosphate; N3-methyl-8-oxo-2′-deoxy-isoinosine-3′-monophosphate; or N1,N3-dimethyl-8-oxo-2′-deoxy-isoinosine-3′-monophosphate.

In some embodiments a nucleobase may comprise xanthine.

In some embodiments a mono-nucleoside substance or mono-nucleotide substance may comprise a xanthine nucleobase, wherein the nucleobase may be a modified or unmodified nucleobase.

A mono-nucleoside or mono-nucleotide comprising a xanthine nucleobase is herein referred to as a xanthosine-mono-nucleoside or xanthosine-mono-nucleotide.

In some embodiments a mono-nucleoside substance or mono-nucleotide substance may comprise a modified or unmodified xanthosine-mono-nucleoside or a modified or unmodified xanthosine-mono-nucleotide.

In some embodiments non-limiting examples of a modified or unmodified xanthine nucleobase may include: xanthine; N1-methyl-xanthine; N3-methyl-xanthine; N7-methyl-xanthine; 8-oxo-xanthine; 8-hydroxy-xanthine; N1,N3-dimethyl-xanthine; N3,N7-dimethyl-xanthine; N1,N7-dimethyl-xanthine; N1,N3,N7-trimethyl-xanthine; N1-methyl-8-oxo-xanthine; N3-methyl-8-oxo-xanthine; or N1,N3-dimethyl-8-oxo-xanthine.

In some embodiments non-limiting examples of a modified or unmodified xanthosine-mono-nucleoside may include: xanthosine; N1-methyl-xanthosine; N3-methyl-xanthosine; N7-methyl-xanthosine; 8-oxo-xanthosine; 8-hydroxy-xanthosine; N1,N3-dimethyl-xanthosine; N3,N7-dimethyl-xanthosine; N1,N7-dimethyl-xanthosine; N1,N3,N7-trimethyl-xanthosine; N1-methyl-8-oxo-xanthosine; N3-methyl-8-oxo-xanthosine; N1,N3-dimethyl-8-oxo-xanthosine; 2′-deoxyxanthosine; N1-methyl-2′-deoxyxanthosine; N3-methyl-2′-deoxyxanthosine; N7-methyl-2′-deoxyxanthosine; 8-oxo-2′-deoxyxanthosine; 8-hydroxy-2′-deoxyxanthosine; N1,N3-dimethyl-2′-deoxyxanthosine; N3,N7-dimethyl-2′-deoxyxanthosine; N1,N7-dimethyl-2′-deoxyxanthosine; N1,N3,N7-trimethyl-2′-deoxyxanthosine; N1-methyl-8-oxo-2′-deoxyxanthosine; N3-methyl-8-oxo-2′-deoxyxanthosine; N1,N3-dimethyl-8-oxo-2′-deoxyxanthosine; 2′-O-methylxanthosine; N1-methyl-2′-O-methylxanthosine; N3-methyl-2′-O-methylxanthosine; N7-methyl-2′-O-methylxanthosine; 8-oxo-2′-O-methylxanthosine; 8-hydroxy-2′-O-methylxanthosine; N1,N3-dimethyl-2′-O-methylxanthosine; N3,N7-dimethyl-2′-O-methylxanthosine; N1,N7-dimethyl-2′-O-methylxanthosine; N1,N3,N7-trimethyl-2′-O-methylxanthosine; N1-methyl-8-oxo-2′-O-methylxanthosine; N3-methyl-8-oxo-2′-O-methylxanthosine; N1,N3-dimethyl-8-oxo-2′-O-methylxanthosine; 2′,3′,5′-tri-O-acetylxanthosine; N1-methyl-2′,3′,5′-tri-O-acetylxanthosine; N3-methyl-2′,3′,5′-tri-O-acetylxanthosine; N7-methyl-2′,3′,5′-tri-O-acetylxanthosine; 8-oxo-2′,3′,5′-tri-O-acetylxanthosine; 8-hydroxy-2′,3′,5′-tri-O-acetylxanthosine; N1,N3-dimethyl-2′,3′,5′-tri-O-acetylxanthosine; N3,N7-dimethyl-2′,3′,5′-tri-O-acetylxanthosine; N1,N7-dimethyl-2′,3′,5′-tri-O-acetylxanthosine; N1,N3,N7-trimethyl-2′,3′,5′-tri-O-acetylxanthosine; N1-methyl-8-oxo-2′,3′,5′-tri-O-acetylxanthosine; N3-methyl-8-oxo-2′,3′,5′-tri-O-acetylxanthosine; or N1,N3-dimethyl-8-oxo-2′,3′,5′-tri-O-acetylxanthosine.

In some embodiments non-limiting examples of a modified or unmodified xanthosine-mono-nucleotide may include: xanthosine-5′-monophosphate; N1-methyl-xanthosine-5′-monophosphate; N3-methyl-xanthosine-5′-monophosphate; N7-methyl-xanthosine-5′-monophosphate; 8-oxo-xanthosine-5′-monophosphate; 8-hydroxy-xanthosine-5′-monophosphate; N1,N3-dimethyl-xanthosine-5′-monophosphate; N3,N7-dimethyl-xanthosine-5′-monophosphate; N1,N7-dimethyl-xanthosine-5′-monophosphate; N1,N3,N7-trimethyl-xanthosine-5′-monophosphate; N1-methyl-8-oxo-xanthosine-5′-monophosphate; N3-methyl-8-oxo-xanthosine-5′-monophosphate; N1,N3-dimethyl-8-oxo-xanthosine-5′-monophosphate; xanthosine-3′-monophosphate; N1-methyl-xanthosine-3′-monophosphate; N3-methyl-xanthosine-3′-monophosphate; N7-methyl-xanthosine-3′-monophosphate; 8-oxo-xanthosine-3′-monophosphate; 8-hydroxy-xanthosine-3′-monophosphate; N1,N3-dimethyl-xanthosine-3′-monophosphate; N3,N7-dimethyl-xanthosine-3′-monophosphate; N1,N7-dimethyl-xanthosine-3′-monophosphate; N1,N3,N7-trimethyl-xanthosine-3′-monophosphate; N1-methyl-8-oxo-xanthosine-3′-monophosphate; N3-methyl-8-oxo-xanthosine-3′-monophosphate; N1,N3-dimethyl-8-oxo-xanthosine-3′-monophosphate; 2′-deoxyxanthosine-5′-monophosphate; N1-methyl-2′-deoxyxanthosine-5′-monophosphate; N3-methyl-2′-deoxyxanthosine-5′-monophosphate; N7-methyl-2′-deoxyxanthosine-5′-monophosphate; 8-oxo-2′-deoxyxanthosine-5′-monophosphate; 8-hydroxy-2′-deoxyxanthosine-5′-monophosphate; N1,N3-dimethyl-2′-deoxyxanthosine-5′-monophosphate; N3,N7-dimethyl-2′-deoxyxanthosine-5′-monophosphate; N1,N7-dimethyl-2′-deoxyxanthosine-5′-monophosphate; N1,N3,N7-trimethyl-2′-deoxyxanthosine-5′-monophosphate; N1-methyl-8-oxo-2′-deoxyxanthosine-5′-monophosphate; N3-methyl-8-oxo-2′-deoxyxanthosine-5′-monophosphate; N1,N3-dimethyl-8-oxo-2′-deoxyxanthosine-5′-monophosphate; 2′-deoxyxanthosine-3′-monophosphate; N1-methyl-2′-deoxyxanthosine-3′-monophosphate; N3-methyl-2′-deoxyxanthosine-3′-monophosphate; N7-methyl-2′-deoxyxanthosine-3′-monophosphate; 8-oxo-2′-deoxyxanthosine-3′-monophosphate; 8-hydroxy-2′-deoxyxanthosine-3′-monophosphate; N1,N3-dimethyl-2′-deoxyxanthosine-3′-monophosphate; N3,N7-dimethyl-2′-deoxyxanthosine-3′-monophosphate; N1,N7-dimethyl-2′-deoxyxanthosine-3′-monophosphate; N1,N3,N7-trimethyl-2′-deoxyxanthosine-3′-monophosphate; N1-methyl-8-oxo-2′-deoxyxanthosine-3′-monophosphate; N3-methyl-8-oxo-2′-deoxyxanthosine-3′-monophosphate; or N1,N3-dimethyl-8-oxo-2′-deoxyxanthosine-3′-monophosphate.

In some embodiments a mono-nucleoside substance or mono-nucleotide substance may comprise a nucleobase described herein, wherein the nucleobase may be a modified or unmodified nucleobase. As non-limiting examples, a nucleobase may include: a uracil nucleobase, a cytosine nucleobase, an isocytosine nucleobase, a pseudocytosine nucleobase, a pseudoisocytosine nucleobase, a pseudouracil nucleobase, an orotate nucleobase, a guanine nucleobase, an isoguanine nucleobase, an adenine nucleobase, a hypoxanthine nucleobase, an isohypoxanthine nucleobase, a xanthine nucleobase, a pyridine-nucleobase, a 2-pyrimidinone nucleobase, or a 4-pyrimidinone nucleobase.

Non-limiting examples of a pyrimidine nucleobase may include: a uracil nucleobase, a cytosine nucleobase, an isocytosine nucleobase, a pseudocytosine nucleobase, a pseudoisocytosine nucleobase, a pseudouracil nucleobase, an orotate nucleobase, a 2-pyrimidinone nucleobase, or a 4-pyrimidinone nucleobase.

Non-limiting examples of a purine nucleobase may include: a guanine nucleobase, an isoguanine nucleobase, an adenine nucleobase, a hypoxanthine nucleobase, an isohypoxanthine nucleobase, or a xanthine nucleobase.

In some embodiments a mono-nucleoside substance or mono-nucleotide substance may comprise a purine nucleobase, wherein the nucleobase may form an N-glycosidic bond with ribose at the N9 position of the purine ring. As a non-limiting example, a purine nucleobase may be bonded to ribose via a beta-N9-glycosidic bond. In some embodiments a mono-nucleoside substance or mono-nucleotide substance may comprise a pyrimidine nucleobase, wherein the nucleobase may form an N-glycosidic bond with ribose at the N1 position of the pyrimidine ring. As a non-limiting example, a pyrimidine nucleobase may be bonded to ribose via a beta-N1-glycosidic bond. In some embodiments a mono-nucleoside substance or mono-nucleotide substance may comprise a pyrimidine nucleobase, wherein the nucleobase may form a C5-glycoside via a C—C bond with ribose at the C5 position of the pyrimidine ring. As a non-limiting example, a pyrimidine nucleobase may be bonded to ribose via a C5-C1′ bond forming a C5-glycoside. In some embodiments a mono-nucleoside substance or mono-nucleotide substance may comprise a pyridine nucleobase, wherein the nucleobase may form an N-glycosidic bond with ribose at the N1 position of the pyridine ring. As a non-limiting example, a pyridine nucleobase may be bonded to ribose via a beta-N1-glycosidic bond.

As a non-limiting example, a mono-nucleoside substance or a mono-nucleotide substance may comprise ribose (e.g. beta-D-ribose) wherein the anomeric carbon of ribose may be bonded to a nucleobase. As a non-limiting example, a mono-nucleoside substance or a mono-nucleotide substance may comprise beta-D-ribose wherein the anomeric carbon of beta-D-ribose may be bonded to one of a purine or pyrimidine nucleobase.

In some embodiments a mono-nucleoside substance or a mono-nucleotide substance may comprise ribose bonded to a nucleobase. In some embodiments a mono-nucleoside substance or a mono-nucleotide substance may comprise beta-D-ribose bonded to a nucleobase. In some embodiments a mono-nucleoside substance or a mono-nucleotide substance may comprise ribose bonded to a nucleobase, wherein ribose may be alpha-D-ribose, beta-D-ribose, alpha-L-ribose, or beta-L-ribose.

As used herein, the term ribose refers to ribofuranose. As a non-limiting example, ribose may include alpha-D-ribofuranose, beta-D-ribofuranose, alpha-L-ribofuranose, or beta-L-ribofuranose.

In some embodiments a mono-nucleoside substance or mono-nucleotide substance may comprise a different type of aldopentose (e.g. an aldopentofuranose) substituted for ribose. As a non-limiting example, a mono-nucleoside substance or a mono-nucleotide substance may comprise a different type of aldopentose (e.g. an aldopentofuranose) selected from arabinofuranose, xylofuranose, or lyxofuranose, wherein a different type of aldopentose may be substituted for ribose. As a non-limiting example, an aldopentose may be an alpha-D, beta-D, alpha-L, or beta-L aldopentose.

In some embodiments a mono-nucleoside substance may comprise a modified mono-nucleoside, wherein a modified mono-nucleoside may comprise one or more ribose modifications. Non-limiting example modified mono-nucleoside ribose modifications may include where one or more hydroxy group on the ribose sugar may be substituted with one or more of the following substituents: hydrogen (H), methoxy (—O—CH₃), ethoxy (—O—CH₂—CH₃), or acetoxy (—O—(C═O)—CH₃).

In some embodiments a mono-nucleoside substance may comprise a modified mono-nucleoside, where ribose may have up to 3 hydroxy groups substituted with a substituent (e.g. 1, 2, or 3 substituents), up to 2 hydroxy groups substituted with a substituent (e.g. 1 or 2 substituents), or up to 1 hydroxy group substituted with a substituent; wherein the substituents may be the same or different. A non-limiting example of a mono-nucleoside substance comprising ribose wherein ribose may have up to 3 substituents is 2′,3′,5′-tri-O-acetyl-uridine.

As a non-limiting example, a mono-nucleoside substance may comprise one of the following bonded to a nucleobase: ribose, 2′-deoxyribose; 3′-deoxyribose; 2′,3′-dideoxyribose; 2′-O-methyl-ribose; 2′-O-methyl-3′-deoxyribose; 3′-O-methyl-ribose; 3′-O-methyl-2′-deoxyribose; 2′,3′-O-dimethyl-ribose; 5′-O-methyl-ribose; 5′-O-methyl-2′-deoxyribose; 5′-O-methyl-2′,3′-dideoxyribose; 2′,5′-O-dimethyl-ribose; 2′-O-acetylribose; 3′-O-acetylribose; 5′-O-acetylribose; 2′,3′-di-O-acetylribose; 2′,5′-di-O-acetylribose; 3′,5′-di-O-acetylribose; 2′,3′,5′-tri-O-acetylribose; 3′-O-acetyl-2′-deoxyribose; 5′-O-acetyl-2′-deoxyribose; 3′,5′-di-O-acetyl-2′-deoxyribose; 3′-O-acetyl-2′-O-methyl-ribose; 5′-O-acetyl-2′-O-methyl-ribose; or 3′,5′-di-O-acetyl-2′-O-methyl-ribose, as non-limiting examples.

In some embodiments a mono-nucleotide substance may comprise a modified mono-nucleotide, wherein a modified mono-nucleotide may comprise one or more ribose modifications. Non-limiting example modified mono-nucleotide ribose modifications may include where one or more hydroxy group on the ribose sugar may be substituted with one or more of the following substituents: hydrogen (H), methoxy (—O—CH₃), ethoxy (—O—CH₂—CH₃), acetoxy (—O—(C═O)—CH₃), phosphate (—O—PO₃²⁻), diphosphate ((—O—PO₂⁻)—(—O—PO₃²⁻)), triphosphate ((—O—PO₂⁻)₂—(—O—PO₃²⁻)), or cyclic-monophosphate (e.g. 3′,5′-cyclic monophosphate, or 2′,3′-cyclic monophosphate, described in Formula 1-B and Formula 1-C); such that at least one hydroxy group is substituted with at least one phosphate group (e.g. phosphate, diphosphate, triphosphate, or cyclic-monophosphate).

In some embodiments a mono-nucleotide substance may comprise a modified mono-nucleotide, where ribose may have up to 3 hydroxy groups substituted with a substituent (e.g. 1, 2, or 3 substituents), or up to 2 hydroxy groups substituted with a substituent (e.g. 1 or 2 substituents); wherein the substituents may be the same or different, such that at least one hydroxy group is substituted with at least one phosphate group. A non-limiting example of a mono-nucleotide substance comprising ribose wherein ribose may have up to 3 substituents is 2′-deoxyguanosine-3′,5′-cyclic-monophosphate. Another non-limiting example of a mono-nucleotide substance comprising ribose wherein ribose may have up to 2 substituents is 2′-deoxyinosine-5′-monophosphate.

As a non-limiting example, a mono-nucleotide substance may comprise one of the following bonded to a nucleobase: ribose-5′-monophosphate; ribose-3′-monophosphate; ribose-2′-monophosphate; ribose-3′,5′-bismonophosphate; ribose-2′,5′-bismonophosphate; ribose-2′,3′-cyclic monophosphate; ribose-3′,5′-cyclic monophosphate; 2′-deoxy-ribose-5′-monophosphate; 2′-deoxy-ribose-3′-monophosphate; 2′-deoxy-ribose-3′,5′-bismonophosphate; 3′-deoxy-ribose-5′-monophosphate; 2′,3′-dideoxy-ribose-5′-monophosphate; 5′-deoxy-ribose-3′-monophosphate; 2′,5′-dideoxy-ribose-3′-monophosphate; 2′-O-methyl-ribose-5′-monophosphate; 2′-O-methyl-ribose-3′-monophosphate; 2′-O-methyl-ribose-3′,5′-bismonophosphate; 3′-O-methyl-ribose-5′-monophosphate; 2′,3′-O-dimethyl-ribose-5′-monophosphate; 5′-O-methyl-ribose-3′-monophosphate; 2′,5′-O-dimethyl-ribose-3′-monophosphate; 5′-O-methyl-ribose-2′-monophosphate; 3′-O-methyl-ribose-2′-monophosphate; 5′-O-methyl-ribose-2′,3′-cyclic monophosphate; 5′-deoxyribose-2′,3′-cyclic monophosphate; 2′-O-methyl-ribose-3′,5′-cyclic monophosphate; 2′-deoxyribose-3′,5′-cyclic monophosphate; 2′-O-methyl-3′-deoxy-ribose-5′-monophosphate; 3′-O-methyl-2′-deoxy-ribose-5′-monophosphate; 2′-O-methyl-5′-deoxy-ribose-3′-monophosphate; 5′-O-methyl-2′-deoxy-ribose-3′-monophosphate; ribose-5′-diphosphate; ribose-3′-diphosphate; ribose-5′-triphosphate; or ribose-3′-triphosphate, as non-limiting examples.

In some embodiments a composition may comprise one or more RNA stabilizing substance (such as one or more mono-nucleoside substance or one or more mono-nucleotide substance, as non-limiting examples) and one or more RNA substance, wherein the RNA substance may be a polymeric RNA. In some embodiments a composition may comprise one or more RNA stabilizing substance (such as one or more mono-nucleoside substance or one or more mono-nucleotide substance, as non-limiting examples) and one or more RNA substance, wherein the RNA substance may be a polymeric RNA comprising a polymer of at least 10 or more nucleotides, or at least 20 or more nucleotides, or at least 50 or more nucleotides, or at least 100 or more nucleotides, or at least 200 or more nucleotides, or at least 500 or more nucleotides, or at least 1,000 or more nucleotides, or at least 1,500 or more nucleotides, or at least 2,000 or more nucleotides.

In some embodiments a composition may comprise one or more RNA stabilizing substance (such as one or more mono-nucleoside substance or one or more mono-nucleotide substance, as non-limiting examples) and one or more RNA substance, wherein the RNA substance may be an RNA that has been at least partially purified (such as an RNA substance that has undergone at least one or more purification steps as a non-limiting example).

In some embodiments a composition may comprise one or more RNA stabilizing substance (such as one or more mono-nucleoside substance or one or more mono-nucleotide substance, as non-limiting examples) and one or more RNA substance, wherein the RNA substance may be a polymeric RNA that has been at least partially purified after the RNA has been synthesized (including chemical synthesis, transcription (including in vitro transcription), cellular synthesis (including synthesis inside of an organism, plant, microbe, yeast, bacteria, eukaryotic cell, or prokaryotic cell), as non-limiting examples).

As a non-limiting example, an RNA or RNA substance that has been purified or at least partially purified may be a polymeric RNA that has undergone at least one or more purification steps (such as, to at least partially remove or reduce the amount of one or more cellular materials or components used during RNA synthesis, as non-limiting examples). As a non-limiting example, an RNA or RNA substance that has been purified or at least partially purified may be a polymeric RNA that has undergone at least one or more purification steps, wherein one or more purification steps may include, chromatography (including, but not limited to solid phase extraction chromatography, liquid phase extraction chromatography, supported liquid phase extraction chromatography, liquid chromatography, FPLC, HPLC, spin columns, silica or silica gel, centrifugal partition chromatography, column chromatography, reverse phase chromatography, ion exchange chromatography, cation exchange chromatography, anion exchange chromatography, size exclusion chromatography or gel-filtration chromatography, affinity chromatography, or combinations thereof), extraction (including, but not limited to, liquid-liquid extraction, phenol/chloroform extraction or extraction using organic solvents, as non-limiting examples), homogenization, treatment with an enzyme (including, but not limited to, DNase, proteinase, or specific RNase (e.g. RNase H, RNaseIII or other RNase that may degrade one or more types of RNA, as non-limiting examples)) or combinations thereof), crystallization, precipitation, dialysis, desalting, buffer exchange, centrifugation, filtration (including tangential flow filtration, vacuum filtration, or gradient or stepwise filtration, as non-limiting examples), gel electrophoresis, fractionation, evaporation, distillation, adsorption, or combinations thereof, as non-limiting examples.

As non-limiting examples, an at least partially purified RNA substance may be a polymeric RNA wherein following synthesis at least 5% of all non-polymeric RNA components present during synthesis (excluding water) have been removed (as measured on a weight-by-weight basis). As non-limiting examples, an at least partially purified RNA substance may be a polymeric RNA wherein following synthesis at least 5%, or at least 10%, or at least 15%, or at least 20%, or at least 25%, or at least 30%, or at least 40%, or at least 50%, or at least 60%, or at least 70%, or at least 75%, or at least 80%, or at least 85%, or at least 90%, or at least 95% of all non-polymeric RNA components present during synthesis (excluding water) have been removed (as measured on a weight-by-weight basis). Non-limiting examples of one or more non-polymeric RNA components that may be present during RNA synthesis, where one or more non-polymeric RNA component may be at least one of reduced or partially removed by at least one or more purification steps, may include membranes, lipids, proteins, aggregates, lipopolysaccharide, endotoxin, polysaccharide, DNA, unincorporated nucleotides, nucleosides, or nucleobases, ribose sugars, amidites, phosphoramidites, enzymes, buffers, salts, ions, or organic solvents (such as acetonitrile, DMSO, methanol, ethanol, dichloromethane, acetone, trifluoroacetic acid, N-methyl-2-pyrrolidone, phenol, chloroform, or N,N-dimethylformamide, as non-limiting examples).

As another non-limiting example, an RNA stabilizing composition comprising an at least partially purified RNA substance (such as a polymeric RNA that has been at least partially purified following synthesis in a bacterial or microbial cell, as a non-limiting example) may have an endotoxin level less than 100,000,000 endotoxin units/mL; or less than 10,000,000 endotoxin units/mL; or less than 1,000,000 endotoxin units/mL; or less than 100,000 endotoxin units/mL; or less than 10,000 endotoxin units/mL; or less than 1,000 endotoxin units/mL; or less than 500 endotoxin units/mL; or less than 200 endotoxin units/mL; or less than 100 endotoxin units/mL; or less than 50 endotoxin units/mL; or less than 20 endotoxin units/mL; or less than 10 endotoxin units/mL; or less than 5 endotoxin units/mL; or less than 2 endotoxin units/mL; or less than 1 endotoxin units/mL. One of ordinary skill in the art would appreciate that endotoxin testing is known art, as a non-limiting example, endotoxin level may be measured using a limulus amebocyte lysate (LAL) assay, or other suitable method.

In some embodiments a composition may comprise one or more mono-nucleoside substance or one or more mono-nucleotide substance and one or more RNA substance, wherein a mono-nucleoside substance or a mono-nucleotide substance may be an exogenous mono-nucleoside or an exogenous mono-nucleotide. As a non-limiting example, an exogenous mono-nucleoside or an exogenous mono-nucleotide may be a mono-nucleoside or a mono-nucleotide wherein the mono-nucleoside or mono-nucleotide may be added to (e.g. mixed with or otherwise combined with) a polymeric RNA substance after the RNA has been synthesized (such as chemical synthesis, cellular synthesis, or transcription, as non-limiting examples) or at least partially purified, to produce a composition comprising one or more RNA stabilizing substance and one or more RNA substance where the total amount (e.g. total weight) of all nucleosides or all nucleotides within the composition is increased compared to the total amount of nucleosides or nucleotides within the composition prior to the addition of an exogenous mono-nucleoside or exogenous mono-nucleotide.

In some embodiments a composition may comprise an RNA stabilizing substance (such as a mono-nucleoside substance or a mono-nucleotide substance, as non-limiting examples) and one or more RNA substance, wherein the composition may be free of or substantially free of RNA polymerase. In some embodiments a composition may comprise an RNA stabilizing substance (such as a mono-nucleoside substance or a mono-nucleotide substance, as non-limiting examples) and one or more RNA substance, wherein the total amount of RNA polymerase may be less than about 1 picogram (pg), or less than about 10 pg, or less than about 100 pg, or less than about 1 nanogram (ng), or less than about 2 ng, or less than about 5 ng, or less than about 10 ng, or less than about 20 ng, or less than about 50 ng, or less than about 100 ng, or less than about 200 ng, or less than about 500 ng, or less than about 1 μg, or less than about 2 μg, or less than about 5 μg, or less than about 10 μg, or less than about 20 μg, or less than about 50 μg.

In some embodiments a composition may comprise one or more RNA substance and one or more RNA stabilizing substance, where the RNA stabilizing substance may comprise a mono-nucleoside substance or a mono-nucleotide substance. In some embodiments an RNA stabilizing substance comprising a mono-nucleoside substance or a mono-nucleotide substance may be used in or to produce one or more RNA stabilizing composition described herein.

Embodiments comprising mono-nucleoside substances or mono-nucleotide substances, may include one or more conjugate acid or conjugate base, tautomer, stereoisomer, or salt thereof.

In some embodiments an RNA stabilizing substance comprising a mono-nucleoside substance or a mono-nucleotide substance may be used in or to produce one or more RNA stabilizing composition described herein, where the concentration of a mono-nucleoside substance or a mono-nucleotide substance may be between about 5 mM-1M, or between about 20 mM-1M, or between about 20 mM-500 mM, as non-limiting examples (e.g. about 5 mM, 10 mM, 20 mM, 50 mM, 100 mM, 150 mM, 200 mM, 250 mM, 300 mM, 400 mM, 500 mM, 600 mM, 800 mM, or 1M, as non-limiting examples).

In some embodiments an RNA stabilizing substance comprising a mono-nucleoside substance or a mono-nucleotide substance may be used in or to produce one or more RNA stabilizing composition described herein, where the concentration of a mono-nucleoside substance or a mono-nucleotide substance may be at least 1 mM or greater, or may be at least 5 mM or greater, or may be at least 10 mM or greater, or may be at least 20 mM or greater, or may be at least 30 mM or greater, or may be at least 40 mM or greater, or may be at least 50 mM or greater, or may be at least 100 mM or greater.

In some embodiments an RNA stabilizing substance comprising a mono-nucleoside substance or a mono-nucleotide substance may be used in or to produce one or more RNA stabilizing composition described herein, where the total concentration of all mono-nucleosides or all mono-nucleotides in a composition may be at least 1 mM or greater, or may be at least 5 mM or greater, or may be at least 10 mM or greater, or may be at least 20 mM or greater, or may be at least 30 mM or greater, or may be at least 40 mM or greater, or may be at least 50 mM or greater, or may be at least 100 mM or greater.

In some embodiments an RNA stabilizing substance comprising a mono-nucleoside substance or a mono-nucleotide substance may be used in or to produce one or more RNA stabilizing composition described herein, where the total amount (e.g. total weight) of all mono-nucleosides or all mono-nucleotides in a composition may be at least 25 μg or greater, or may be at least 50 μg or greater, or may be at least 100 μg or greater, or may be at least 250 μg or greater, or may be at least 500 μg or greater, or may be at least 1 mg or greater, or may be at least 2 mg or greater, or may be at least 5 mg or greater, or may be at least 10 mg or greater, or may be at least 25 mg or greater, or may be at least 50 mg or greater.

In some embodiments an RNA stabilizing substance comprising a mono-nucleoside substance or a mono-nucleotide substance may be used in or to produce one or more RNA stabilizing composition described herein, where the total concentration of all mono-nucleosides or all mono-nucleotides in a composition may be at least 1 mg/mL or greater, or may be at least 2 mg/mL or greater, or may be at least 5 mg/mL or greater, or may be at least 10 mg/mL or greater, or may be at least 15 mg/mL or greater, or may be at least 20 mg/mL or greater, or may be at least 25 mg/mL or greater, or may be at least 30 mg/mL or greater, or may be at least 40 mg/mL or greater, or may be at least 50 mg/mL or greater, or may be at least 100 mg/mL or greater.

In some embodiments an RNA stabilizing substance comprising a mono-nucleoside substance or a mono-nucleotide substance may be used in or to produce one or more RNA stabilizing composition described herein, where the total weight percent of all mono-nucleosides or all mono-nucleotides in a composition may be at least 0.01% or greater, or may be at least 0.02% or greater, or may be at least 0.05% or greater, or may be at least 0.1% or greater, or may be at least 0.2% or greater, or may be at least 0.5% or greater, or may be at least 1.0% or greater, or may be at least 2.0% or greater, or may be at least 3.0% or greater, or may be at least 4.0% or greater, or may be at least 5.0% or greater.

In some embodiments a composition comprising a mono-nucleoside substance or a mono-nucleotide substance may be used in or to produce an RNA stabilizing composition comprising a gel (such as a hydrogel as a non-limiting example) or viscous fluid comprising water (such as a thixotropic fluid comprising water as a non-limiting example) and one or more mono-nucleoside substance or one or more mono-nucleotide substance and one or more RNA substance. Formation of gels or viscous fluids, including hydrogels, by mono-nucleosides or mono-nucleotides is known in the art. As a non-limiting example, guanosine mono-nucleosides or mono-nucleotides may form a gel or viscous fluid comprising water through the assembly of a network of monomers assembled via hydrogen bonds, electrostatic interactions, or base stacking.

The inventors have surprisingly discovered the novel configuration of combining one or more RNA substance with one or more mono-nucleoside substance or one or more mono-nucleotide substance to create one or more compositions that improve RNA stability, wherein a mono-nucleoside substance or a mono-nucleotide substance may form a gel or viscous fluid comprising water (such as a thixotropic fluid comprising water) in the presence of one or more RNA substance. In some embodiments one or more RNA stabilizing compositions described herein may comprise one or more mono-nucleoside substance or one or more mono-nucleotide substance and one or more RNA substance, wherein a mono-nucleoside substance or a mono-nucleotide substance may form a gel (such as a hydrogel as a non-limiting example) or viscous fluid comprising water (such as a thixotropic fluid comprising water as a non-limiting example). As a non-limiting example, a gel or viscous fluid comprising water (e.g. a thixotropic fluid) may comprise a network of monomers comprising one or more mono-nucleoside substance or one or more mono-nucleotide substance. As a non-limiting example, a network of monomers comprising one or more mono-nucleoside substance or one or more mono-nucleotide substance may comprise a supramolecular assembly of individual monomers into structures (such as helices, tubes, rods, sheets, rings, crystals, or fibrils, as non-limiting examples) assembled via hydrogen bonds, base stacking, or electrostatic interactions.

As a non-limiting example, mono-nucleoside substances or mono-nucleotide substances (such as guanosine-5′-monophosphate, as a non-limiting example) may be used to produce compositions comprising a network of monomers assembled via hydrogen bonds, electrostatic interactions, or base stacking with gel-like properties (such as allowing the diffusion of water or ions through a viscous fluid or ionically bonded network, as a non-limiting example), wherein the network of monomers or viscosity of the composition may be adjusted by changing the pH, or changing the ionic strength, or increasing or decreasing the concentration of one or more constituents within a composition.

As a non-limiting example, these networks of monomers comprising one or more mono-nucleoside substance or one or more mono-nucleotide substance may be fully or partially reversible or may be reassembled in selected ways to produce tunable (e.g. reversible, semi-reversible, or selectively adjustable, as non-limiting examples) compositions or RNA storage environments comprising one or more RNA substance and one or more RNA stabilizing substance. Without being bound to a particular theory or mode action, the inventors believe that the formation of these networks of monomers, comprising one or more mono-nucleoside or mono-nucleotide assembled via hydrogen bonds, base stacking, or electrostatic interactions, may substantially reduce the movement or flexibility of one or more RNA substances within a composition or storage environment and thereby restrict conformational changes that may promote RNA hydrolysis or degradation.

In some embodiments one or more RNA stabilizing composition described herein may comprise at least one of a gel, hydrogel, or viscous fluid comprising water (such as a thixotropic fluid comprising water as a non-limiting example). In some embodiments one or more RNA stabilizing composition described herein may comprise one or more mono-nucleoside substance or one or more mono-nucleotide substance and one or more RNA substance, wherein the composition may comprise at least one of a gel, hydrogel, or viscous fluid comprising water (such as a thixotropic fluid comprising water as a non-limiting example). In some embodiments one or more RNA stabilizing composition comprising one or more mono-nucleoside substance or one or more mono-nucleotide substance and one or more RNA substance, wherein the composition may comprise a gel, hydrogel, or viscous fluid comprising water (such as a thixotropic fluid comprising water as a non-limiting example), may also comprise one or more additional RNA stabilizing substance. In some embodiments one or more RNA stabilizing composition comprising one or more mono-nucleoside substance or one or more mono-nucleotide substance and one or more RNA substance, wherein the composition may comprise a gel, hydrogel, or viscous fluid comprising water (such as a thixotropic fluid comprising water as a non-limiting example), may also comprise one or more of one or more additional substance, such as an RNA stabilizing substance, additive substance, inorganic cation (or salt thereof), cellular uptake agent, or water.

In some embodiments a composition comprising one or more mono-nucleoside substance or one or more mono-nucleotide substance and one or more RNA substance may have a selectively adjustable viscosity wherein the viscosity of the composition may be selectively adjusted using non-thermal methods, such as changing the pH, or changing the ionic strength, or increasing or decreasing the concentration of one or more constituents within a composition (such as increasing or decreasing the concentration of one or more RNA stabilizing substance, additive substance, or one or more inorganic ions, as non-limiting examples). As a non-limiting example, a composition comprising a mono-nucleoside substance or a mono-nucleotide substance and one or more RNA substance may comprise a high viscosity gel or thixotropic fluid comprising water at a pH of about 5-6 and then may comprise a low viscosity liquid when the pH is then increased to a pH of about 7 or higher. As another non-limiting example, a composition comprising a mono-nucleoside substance or a mono-nucleotide substance and one or more RNA substance may comprise a low viscosity liquid at a low concentration of a selected inorganic cation (such as K⁺ as a non-limiting example) but then may comprise a high viscosity gel or thixotropic fluid comprising water upon increasing the concentration of a selected inorganic cation.

In some embodiments one or more RNA stabilizing composition may comprise a gel (such as hydrogel) or thixotropic fluid comprising water. In some embodiments one or more RNA stabilizing composition may have a selectively adjustable viscosity, where the viscosity of a composition may be adjusted by one or more non-thermal methods described herein. As a non-limiting example, an RNA stabilizing composition with a selectively adjustable viscosity may be capable of converting from a low viscosity liquid to a gel or thixotropic fluid comprising water and then reverting to a low viscosity liquid using non-thermal methods such as by adjusting the pH or concentration of one or more inorganic cations in the composition.

In some embodiments one or more RNA stabilizing composition described herein, may comprise one or more mono-nucleoside substance or one or more mono-nucleotide substance and one or more RNA substance wherein the composition may comprise a gel, hydrogel, or viscous fluid comprising water (such as a thixotropic fluid as a non-limiting example) in the presence of one or more inorganic cation. Non-limiting example inorganic cations may include one of the following: Li, Na, K, Rb, Cs, Ag, Au, Pt, Ti, Cu, NH₄, Mg, Mn, Zn, Fe, Co, Ca, or Ni, or salts thereof (including inorganic and organic salts comprising one or more inorganic cation). In some embodiments one or more RNA stabilizing composition described herein, may comprise one or more mono-nucleoside substance or one or more mono-nucleotide substance and one or more RNA substance wherein the composition may also comprise one or more inorganic cation such as (Li, Na, K, Cs, Ag, Au, Pt, Ti, Rb, NH₄, Mg, Mn, or Zn, as non-limiting examples) or salt thereof (including inorganic and organic salts comprising one or more inorganic cation). In some embodiments one or more RNA stabilizing composition described herein, may comprise one or more mono-nucleoside substance or one or more mono-nucleotide substance and one or more RNA substance, wherein the composition may also comprise one or more inorganic cation where the inorganic cation concentration may be at least 1 mM or greater, or may be at least 5 mM or greater, or may be at least 10 mM or greater, or may be at least 25 mM or greater, or may be at least 50 mM or greater, or may be at least 75 mM or greater, or may be at least 100 mM or greater, or may be at least 125 mM or greater, or may be at least 150 mM or greater, or may be at least 200 mM or greater.

Inorganic cations described herein may include one or more salts comprising an inorganic cation (including inorganic and organic salts comprising one or more inorganic cation). Non-limiting examples of a salt comprising an inorganic cation may include: KCl, potassium acetate, potassium glutamate, potassium aspartate, or potassium citrate.

### Compositions and Viscosity

Methods comprising producing compositions comprising at least one RNA stabilizing substance and at least one RNA substance may comprise the step of combining a first RNA stabilizing substance with at least one RNA substance and may further comprise the step of mixing an additional substance such as a second RNA stabilizing substance or an additional substance (such as a substance comprising an inorganic cation or a substance with specified pH value) that may change at least one physical property of the composition to a different value. Non-limiting examples of physical properties that may change comprise changing the viscosity or changing the rheology to contribute to the composition or mixture being a gel or thixotropic.

Non-limiting embodiments comprising methods of combining or mixing one or more substance described herein may be independent of the order in which each substance may be combined or mixed together. The non-limiting example described above describes steps, not necessarily the sequence of steps that the method comprises. As a non-limiting example, the step of mixing at least one substance that may change at least one physical property may occur after or before mixing the first RNA stabilizing substance with at least one RNA substance.

Methods comprising producing compositions comprising at least one RNA stabilizing substance and at least one RNA substance may comprise one or more steps for producing compositions comprising at least one RNA stabilizing substance and at least one RNA substance wherein the viscosity of the composition may be changed from a first viscosity value or range to a second viscosity value or range. The produced composition may also comprise one or more substances wherein the viscosity may change from the second viscosity to a third viscosity in a selected third viscosity value or range different from the second viscosity and that may be different from or approximately the same as the first viscosity. Methods of producing RNA stabilizing compositions or products comprising at least one RNA stabilizing substance may comprise the step of producing a composition with a selectively adjustable viscosity comprising at least one RNA stabilizing substance in a composition that may be mixed with at least one RNA substance wherein the viscosity of the mixture may be changed from a first viscosity value in a first selected viscosity range to a second viscosity in a second viscosity range and may also be changed to a third viscosity in a third viscosity range that may be approximately the same or different from the first viscosity range. The method of producing RNA stabilizing compositions or products comprising at least one RNA stabilizing substance and at least one RNA substance may comprise the step of mixing at least one substance with at least one RNA stabilizing substance or at least one RNA substance to produce a composition comprising a gel or viscous fluid comprising water (such as a thixotropic composition comprising water) when mixed with at least one RNA stabilizing substance and at least one RNA substance.

The method of producing a composition or products comprising RNA stabilizing substances may comprise the steps of producing a composition with a selectively adjustable viscosity comprising at least one RNA stabilizing substance and at least one RNA substance followed by one or more steps comprising setting the viscosity of the composition to a low viscosity value followed by one or more steps comprising moving (non-limiting examples include by pressure difference (such as by pressurizing or vacuum), such as by pumping or pushing, or pouring) at least some of the selectively adjustable viscosity composition into a container or chamber (non-limiting examples include tubes, vials, and syringes) followed by one or more steps comprising setting the viscosity of the composition in the chamber to a high value. As non-limiting examples, the low viscosity and high viscosity may be viscosities of a composition with a selectively adjustable viscosity when in a low viscosity condition and when in a high viscosity condition. As non-limiting examples, the high viscosity may be at least 10× the low viscosity; or may be at least 50× the low viscosity; or may be at least 80× the low viscosity; or may be at least 100× the low viscosity; or may be at least 1000× the low viscosity; or may be at least 10,000× the low viscosity; or may be at least 100,000× the low viscosity.

The present disclosure describes methods for adjusting the viscosity of RNA stabilizing compositions with a selectively adjustable viscosity that do not rely on altering the temperature of the composition. Adjusting the viscosity by adjusting the temperature of the composition are thermal viscosity adjustment methods. Adjusting the viscosity using methods that do not use temperature as the control variable are non-thermal viscosity adjustment methods. Non-limiting examples of non-thermal methods include methods described in this disclosure such as setting the pH to a value in a selected range and setting an inorganic cation concentration to a value in a selected range and using dilution. The temperature may change incidentally when non-thermal methods of viscosity adjustment are used, non-limiting examples may be the temperature of the composition changing when pH is changed or the temperature changing when a liquid is mixed into the composition to change an inorganic cation concentration. Thermal methods of adjusting viscosity set the energy level (temperature) of the composition and may add or remove energy from the composition to increase, decrease, or maintain a temperature and non-limiting examples include using at least in part conductive or convective heat exchange, such with a heat exchange medium (such as warm or cold air or using warm or cold water), or using at least in part radiant heat exchange, such as from at least one heated surface. The non-thermal methods of adjusting viscosity may be used in conjunction with adjusting the temperature of the compositions to change the viscosity. The non-thermal methods for adjusting viscosity have the advantage, compared to the thermal methods that change the temperature, of not subjecting RNA to temperatures that may otherwise degrade RNA materials. As a non-limiting example, if thermal viscosity adjustment methods are used, RNA in compositions with RNA stabilizing substances may, at least in part, reduce the susceptibility of RNA to degrade at warmer temperatures used to achieve a selected viscosity at a selected temperature.

A non-limiting example method of the present disclosure for producing one or more RNA stabilizing compositions with a selectively adjustable viscosity may comprise at least the step of producing a composition comprising at least one RNA stabilizing substance and at least one RNA substance and at least the step of adjusting the viscosity of the composition. As non-limiting examples, viscosity adjustment of a composition with a selectively adjustable viscosity may be done at least in part, by using one or more non-thermal adjusting methods by adjusting the composition to have one or more composition parameters in selected ranges. As non-limiting examples of composition parameters that may be adjusted, the composition may have pH set to be in a selected range or may have an inorganic cation concentration set to be in a selected range. As a non-limiting example, viscosity adjustment may be done at least in part by using a thermal method to set the temperature to be in a selected range. More than one method for adjusting the viscosity may be used, as non-limiting examples more than one non-thermal method for adjusting viscosity may be used or a combination of at least one non-thermal method may be used with at least one thermal method. The method may further comprise the step of maintaining a low viscosity, or adjusting the viscosity to a lower value, during at least production of the RNA stabilizing composition and may comprise using viscosity at a low value (the manufacturing viscosity) during manufacturing of the RNA stabilizing composition. The producing method may further comprise increasing the viscosity of the RNA stabilizing composition while or after it has been packaged (the packaged product viscosity). The packaged product viscosity is higher than the manufacturing viscosity. As non-limiting examples, the packaged product viscosity may be at least 2× greater; or may be at least 5× greater; or may be at least 10× greater; or may be at least 50× greater; or may be at least 80× greater; or may be at least 100× greater; or may be at least 1000× greater; or may be at least 10,000× greater; or may be at least 100,000× greater, than the manufacturing viscosity. A production and shipping method may further comprise at least in part storing or at least in part shipping or transporting the packaged RNA stabilizing composition with a viscosity at least about (as a non-limiting example, at least about 50%) of the packaged product viscosity.

Additionally, the method may further comprise the step of adjusting the viscosity to a lower value prior to use, such as readjusting the viscosity of the composition to a low value approximately the same as to the manufacturing viscosity, as a non-limiting example. As a non-limiting example, a method may further comprise a step of adjusting the viscosity of an RNA stabilizing composition prior to use, such as by using one or more non-thermal methods or thermal methods or combinations thereof. As a non-limiting example, one or more method may further comprise the step of adjusting the viscosity of a pharmaceutical composition following shipping or storage to a lower value prior to administering at least one of a medicament, vaccine, or therapeutic agent to a subject in need thereof. As a non-limiting example, the viscosity of a composition prior to use may be lower than the packaged viscosity (e.g. high viscosity), wherein the viscosity prior to use may be at least 2× less; or may be at least 5× less; or may be at least 10× less; or may be at least 50× less; or may be at least 80× less; or may be at least 100× less; or may be at least 1000× less; or may be at least 10,000× less; or may be at least 100,000× less than the packaged viscosity.

Non-limiting embodiments of the present disclosure may comprise RNA stabilizing compositions with a selectively adjustable viscosity, wherein the viscosity may be adjusted using non-thermal methods as described herein. Methods for at least one of producing or providing one or more RNA stabilizing composition, may comprise at least one step of adjusting the viscosity of the RNA stabilizing composition, such as by adjusting the pH or adjusting the concentration of one or more inorganic cation.

As a non-limiting example, a method comprising producing a composition may also comprise producing a composition with a selectively adjustable viscosity, where the viscosity of a composition may be adjusted with non-thermal methods by adjusting the pH or adjusting the concentration of at least one inorganic cation in a composition as described herein. As a non-limiting example, a method comprising producing an RNA stabilizing composition may also comprise increasing the viscosity of the composition by adding a substance to decrease the pH or adding an inorganic cation to the composition prior to packaging, shipping, or storage. As a non-limiting example, a method comprising producing an RNA stabilizing composition may also comprise decreasing the viscosity prior to use by increasing the pH or reducing the concentration of an inorganic cation, such as by diluting the composition with a suitable diluent.

As a non-limiting example, a method comprising producing an RNA stabilizing composition with a selectively adjustable viscosity may also comprise placing the composition in a chamber and then increasing the viscosity of the composition by adding a substance or buffer to decrease the pH or adding at least one inorganic cation as described herein.

As a non-limiting example, a method comprising providing a composition may also comprise providing a composition with a selectively adjustable viscosity, where a composition may be provided with a specified viscosity prior to packaging, shipping, or storage. As another non-limiting example, a method comprising providing a composition may also comprise providing a composition with a selectively adjustable viscosity, where a composition may be provided with a specified viscosity (such as a higher viscosity) and then the viscosity may be adjusted to a different specified viscosity (such as lower viscosity) using non-thermal methods prior to use, such as by adjusting the pH or inorganic cation concentration, as described herein.

As non-limiting examples, a method comprising providing a composition with a selectively adjustable viscosity may also comprise providing one or more additional components such as a diluent, including a diluent comprising water (e.g. a buffer or water), to reduce the viscosity prior to use. As another non-limiting example, a method comprising providing a composition with a selectively adjustable viscosity may also comprise providing instructions for use that may include instructions for reducing the viscosity of the composition by adding or mixing a diluent or other substance, such as a provided diluent as a non-limiting example.

In some embodiments of the present disclosure a method may comprise producing one or more RNA stabilizing composition described herein that may have a selectively adjustable viscosity where the viscosity may be increased or decreased using non-thermal methods, such as by the adjusting the pH of the composition or by increasing or decreasing the concentration of one or more inorganic cations or salts thereof (including inorganic or organic salts). As a non-limiting example, one or more method comprising producing an RNA stabilizing composition may also comprise adjusting the viscosity of an RNA stabilizing composition by adding (e.g. mixing or otherwise combining) one or more buffers or substances to adjust the pH to a selected level or range or by adding (e.g. mixing or otherwise combining) one or more inorganic cations or salts thereof, thereby increasing the concentration of one or more inorganic cations, as non-limiting examples. As a non-limiting example, one or more method may comprise a step where the viscosity of a composition may be adjusted (e.g. increased or decreased), such as increasing the viscosity by decreasing the pH or increasing the concentration of an inorganic cation; or decreasing the viscosity by increasing the pH or by diluting or decreasing the concentration of an inorganic cation by adding a diluent comprising water (such as a buffer or water) to the composition.

As a non-limiting example, a method may comprise producing an RNA stabilizing composition with a selectively adjustable viscosity wherein the viscosity of an RNA stabilizing composition may be increased (such as by decreasing the pH or by the addition of or increasing the concentration of one or more inorganic cations, as non-limiting examples) prior to packaging, shipping, or storage. As another non-limiting example, a method may also comprise providing instructions for use or providing one or more component such as a diluent comprising water (e.g. a buffer, water, or other suitable diluent) to reduce the viscosity of the composition prior to the intended use (such as administering a medicament, therapeutic agent, or vaccine, as non-limiting examples).

In some embodiments a method comprising providing one or more RNA stabilizing composition described herein comprising one or more RNA stabilizing substance (e.g. a mono-nucleoside substance or a mono-nucleotide substance, as non-limiting examples) and one or more RNA substance may also comprise providing a composition with a selectively adjustable viscosity. As a non-limiting example, one or more method may comprise providing an RNA stabilizing composition wherein the viscosity of an RNA stabilizing composition may be increased (such as by decreasing the pH or by increasing the concentration of one or more inorganic cations, as non-limiting examples) prior to packaging, shipping, or storage.

As a non-limiting example, one or more method may comprise providing an RNA stabilizing composition wherein the viscosity of an RNA stabilizing composition may be increased (such as by decreasing the pH or by increasing the concentration of one or more inorganic cations, as non-limiting examples) prior to packaging, shipping, or storage and may also comprise providing instructions for use or providing a diluent for reducing the viscosity prior to the intended use (such as administering a medicament, therapeutic agent, or vaccine, as non-limiting examples).

As a non-limiting example, a method may comprise providing an RNA stabilizing composition with a selectively adjustable viscosity wherein the composition may be provided with a high viscosity value prior to at least one of packaging, transporting, shipping, or storage and may also comprise providing a diluent comprising water (e.g. a buffer or water) and instructions for use comprising instructions for mixing the diluent with the composition to reduce the viscosity to a different lower viscosity value prior to the intended use (such as administering a medicament, therapeutic agent, or vaccine, as non-limiting examples).

As a non-limiting example, instructions for use may include one or more instructions described herein, including instructions for mixing one or more substances or components with a composition, or instructions for diluting a composition with a suitable diluent, or instructions for storage or instructions for dosing or administration of one or more composition components. Non-limiting example additional components may include one or more diluents (such as a buffer or diluents comprising water), or one or more additional substances described herein.

As another non-limiting example, one or more method may comprise increasing the viscosity of a composition comprising one or more mono-nucleoside substance or one or more mono-nucleotide substance and one or more RNA substance by decreasing the pH of the composition below about 7 prior to packaging, shipping, or storage and providing instructions for use or additional components, such as one or more buffers, for decreasing the viscosity of the composition by increasing the pH of the composition to about 7 or higher prior to the intended use (such as administering a medicament, therapeutic agent, or vaccine, as non-limiting examples). Another non-limiting example, method may comprise increasing the viscosity of a composition comprising one or more mono-nucleoside substance or one or more mono-nucleotide substance and one or more RNA substance by increasing the concentration of one or more inorganic cation in the composition prior to packaging, shipping, or storage and providing instructions for use or additional components, such as one or more diluents comprising water, to decrease the viscosity of the composition by diluting the composition (e.g. with a diluent comprising water), thereby reducing the concentration one or more inorganic cation, prior to the intended use (such as administering a medicament, therapeutic agent, or vaccine, as non-limiting examples).

As a non-limiting example, the viscosity of one or more RNA stabilizing composition described herein comprising one or more mono-nucleoside substance or one or more mono-nucleotide substance and one or more RNA substance may be selectively adjusted by adjusting the pH of the composition (such as by adding a buffer or other substance to decrease the pH of the composition to a selected value, or by adding a buffer or other substance to increase the pH of the composition to a selected value), or by adjusting the concentration of one or more inorganic cations in the composition (such as by adding one or more inorganic cations or salts thereof to a composition to increase the concentration of one or more inorganic cations in a composition, or by adding a diluent or diluting a composition to decrease the concentration of one or more inorganic cations in a composition).

As a non-limiting example, the viscosity of one or more RNA stabilizing composition described herein comprising one or more mono-nucleoside substance or one or more mono-nucleotide substance and one or more RNA substance may be selectively adjusted (e.g. increased or decreased) by adjusting the pH of the composition to a biocompatible value. As a non-limiting example, a biocompatible value may be a pH of about 5-9. As another non-limiting example, a biocompatible value may be a pH of about 5-9, or a pH of about 6-9, or a pH of about 7-9, or a pH of about 5-8, or a pH of about 6-8, or a pH of about 7-8, or a pH of about 5-7, or a pH of about 6-7, or a pH of about 5-6.

In some embodiments the viscosity of one or more RNA stabilizing composition described herein comprising one or more mono-nucleoside substance or one or more mono-nucleotide substance and one or more RNA substance may be increased by adjusting the pH of the composition to less than about 9, or to less than about 8.5, to less than about 8, or to less than about 7.5, or to less than about 7, or to less than about 6.5, or to less than about 6, or to less than about 5.5. In some embodiments the viscosity of one or more RNA stabilizing composition described herein comprising one or more mono-nucleoside substance or one or more mono-nucleotide substance and one or more RNA substance may be decreased by adjusting the pH of the composition to greater than about 5, or to greater than about 5.5, or to greater than about 6, or to greater than about 6.5, or to greater than about 7.

In some embodiments the viscosity of one or more RNA stabilizing composition described herein comprising one or more mono-nucleoside substance or one or more mono-nucleotide substance and one or more RNA substance may be increased or decreased by adjusting the pH of the composition to a value between about 5-9, or a value between about 5-8.5, or a value between about 6-8.5, or a value between about 6.5-8.5, or a value between about 5-8, or a value between about 5.5-8, or a value between about 6-8, or a value between about 6.5-8, or a value between about 7-8, or a value between about 5-7.5, or a value between about 5.5-7.5, or a value between about 6-7.5, or a value between about 6.5-7.5, or a value between about 5-7, or a value between about 5.5-7, or a value between about 6-7, or a value between about 5-6.5, or a value between about 5-6, or a value between about 5.5-6.5.

In some embodiments the viscosity of one or more RNA stabilizing composition described herein comprising one or more mono-nucleoside substance or one or more mono-nucleotide substance and one or more RNA substance may be increased by increasing the concentration of one or more inorganic cations in the composition, wherein the inorganic cation concentration may be at least 1 mM or greater, or may be at least 5 mM or greater, or may be at least 10 mM or greater, or may be at least 25 mM or greater, or may be at least 50 mM or greater, or may be at least 75 mM or greater, or may be at least 100 mM or greater, or may be at least 125 mM or greater, or may be at least 150 mM or greater, or may be at least 200 mM or greater.

In some embodiments the viscosity of one or more RNA stabilizing composition described herein comprising one or more mono-nucleoside substance or one or more mono-nucleotide substance and one or more RNA substance may be decreased by decreasing the concentration of one or more inorganic cations in the composition, wherein the inorganic cation concentration may be less than bout 200 mM, or may be less than bout 150 mM, or may be less than bout 100 mM, or may be less than bout 50 mM, or may be less than bout 30 mM, or may be less than bout 20 mM, or may be less than bout 10 mM, or may be less than bout 5 mM, or may be less than bout 2 mM, or may be less than bout 1 mM.

As a non-limiting example, one or more method for producing one or more RNA stabilizing composition may comprise increasing the viscosity of one or more RNA stabilizing composition described herein comprising one or more RNA stabilizing substance (e.g. a mono-nucleoside substance or a mono-nucleotide substance) and one or more RNA substance (such as by adding (e.g. mixing or otherwise combining) one or more buffers or substances to decrease the pH or by adding (e.g. mixing or otherwise combining) one or more inorganic cations or salts thereof, thereby increasing the concentration of one or more inorganic cations, as non-limiting examples) prior to packaging, shipping, or storage. Additionally, a method may also comprise one or more additional steps of placing one or more RNA stabilizing composition or components into a chamber and then increasing the viscosity of the composition once inside the chamber, wherein the chamber may be hermetically sealed prior to packaging, shipping, or storage.

Additionally, one or more method described herein may also comprise providing one or more components or instructions for use for reducing viscosity of a composition prior to the intended use (such as administering a medicament, therapeutic agent, or vaccine, as non-limiting examples). As a non-limiting example, one or more method may also comprise providing instructions for use that may include instructions for mixing or combining one or more substances or components (such as a diluent for diluting or adjusting the viscosity of a composition). As another non-limiting example, one or more method may also comprise providing one or more suitable diluent comprising water (such as a buffer to adjust the pH or water) wherein the components may be sterilized or provided in one or more chambers (such as a hermetically sealed chamber). As a non-limiting example, a method may comprise providing a diluent that may comprise a suitable buffer at one or more specified pH value and may also comprise providing instructions for use for mixing the diluent with an RNA stabilizing composition to reduce the viscosity prior to an intended use.

As a non-limiting example, a method comprising producing an RNA stabilizing composition with a selectively adjustable viscosity may also comprise increasing the viscosity of a composition after the composition is placed in a chamber, such as by adding a substance to decrease the pH below a specified value or increasing the concentration of one or more inorganic cation to a specified concentration, as non-limiting examples. As a non-limiting example, one or more method may comprise one or more a steps of increasing the viscosity of a composition inside of a chamber and then hermetically sealing said chamber and at least one of packaging, transporting, shipping, or storing a chamber containing an RNA stabilizing composition.

As a non-limiting example, the viscosity of one or more RNA stabilizing composition with a selectively adjustable viscosity may be adjusted by adjusting the pH of the composition with one or more suitable buffers or buffering agents, described herein.

As another non-limiting example, one or more substances that may be used to adjust viscosity of one or more RNA stabilizing composition described herein may comprise one or more inorganic cations as described herein, or salts thereof (including inorganic and organic salts comprising one or more inorganic cation). Non-limiting examples of one or more substances that may comprise one or more inorganic cations may include: KCl, potassium-citrate, potassium-acetate, potassium-glutamate, potassium-aspartate, or potassium-phosphate, as non-limiting examples.

In some embodiments one or more composition described herein may be thixotropic. As used herein, a thixotropic composition is more viscous under approximately static conditions (low applied force (low shear) conditions) and becomes less viscous when additional external force is applied (higher applied force (greater shear) conditions). Non-limiting examples of low shear may include shear of about 0.01/sec or about 0.1/sec. Non-limiting examples of greater shear may include shear that is at least about 10 times or at least about 100 times the shear at a low shear condition. Non-limiting examples of such additional external force may include forces applied when a composition is agitated, stirred, mixed, pumped, or forced into or through a flow channel. As non-limiting examples for thixotropic compositions described herein, the viscosity at the (higher viscosity) low shear condition compared to the (lower viscosity) higher shear condition may be reduced by at least about 50% or may be reduced by at least about 60%, or may be reduced by at least about 70%, or may be reduced by at least about 80%, or may be reduced by at least about 90%, or may be reduced by at least about 95%, or may be reduced by at least 99%, or may be reduced by about 90% to 95% or by about 90% to 99%.

The viscosities described herein refer to the viscosity of the material at about 20° C.

In some embodiments one or more RNA stabilizing composition described herein comprising one or more RNA stabilizing substance (e.g. a mono-nucleoside substance or a mono-nucleotide substance, as non-limiting examples) and one or more RNA substance, wherein a composition may have a selectively adjustable viscosity (e.g. the viscosity may be increased or decreased by adjusting the pH of the composition or by increasing or decreasing the concentration of one or more inorganic cations, as non-limiting examples), a composition may have a viscosity greater than about 100 centipoise (cP). As non-limiting examples, a composition may have a viscosity greater than about 100 cP; or greater than about 500 cP; or greater than about 1,000 cP; or greater than about 2,000 cP; or greater than about 5,000 cP; or greater than about 10,000 cP; or greater than about 20,000 cP; or greater than about 50,000 cP; or greater than about 100,000 cP; or greater than about 200,000 cP; or greater than about 500,000 cP; or greater than about 1,000,000 cP.

In some embodiments one or more RNA stabilizing composition described herein comprising one or more RNA stabilizing substance (e.g. a mono-nucleoside substance or a mono-nucleotide substance, as non-limiting examples) and one or more RNA substance, wherein a composition may have a selectively adjustable viscosity (e.g. the viscosity may be increased or decreased by adjusting the pH of the composition or by increasing or decreasing the concentration of one or more inorganic cations, as non-limiting examples), a composition may have a viscosity greater than about 100 centipoise (cP) prior to packaging, shipping, or storage. As non-limiting examples, a composition may have a viscosity greater than about 100 cP prior to packaging, shipping, or storage; or greater than about 500 cP prior to packaging, shipping, or storage; or greater than about 1,000 cP prior to packaging, shipping, or storage; or greater than about 2,000 cP prior to packaging, shipping, or storage; or greater than about 5,000 cP prior to packaging, shipping, or storage; or greater than about 10,000 cP prior to packaging, shipping, or storage; or greater than about 20,000 cP prior to packaging, shipping, or storage; or greater than about 50,000 cP prior to packaging, shipping, or storage; or greater than about 100,000 cP prior to packaging, shipping, or storage; or greater than about 200,000 cP prior to packaging, shipping, or storage; or greater than about 500,000 cP prior to packaging, shipping, or storage; or greater than about 1,000,000 cP prior to packaging, shipping, or storage.

In some embodiments one or more RNA stabilizing composition described herein comprising one or more RNA stabilizing substance (e.g. a mono-nucleoside substance or a mono-nucleotide substance, as non-limiting examples) and one or more RNA substance, wherein a composition may have a selectively adjustable viscosity (e.g. the viscosity may be increased or decreased by adjusting the pH of the composition or by increasing or decreasing the concentration of one or more inorganic cations, as non-limiting examples), a composition may have a viscosity less than about 100,000 centipoise (cP) prior to the intended use (such as administering a medicament, therapeutic agent, or vaccine, as non-limiting examples). As non-limiting examples, a composition may have a viscosity less than about 100,000 cP prior to the intended use; or less than about 50,000 cP prior to the intended use; or less than about 10,000 cP prior to the intended use; or less than about 5,000 cP prior to the intended use; or less than about 2,000 cP prior to the intended use; or less than about 1,000 cP prior to the intended use; or less than about 500 cP prior to the intended use; or less than about 200 cP prior to the intended use; or less than about 100 cP prior to the intended use; or less than about 50 cP prior to the intended use.

In other non-limiting embodiments one or more method comprising producing one or more RNA stabilizing composition (e.g. a composition that may have a selectively adjustable viscosity) comprising one or more RNA stabilizing substance (e.g. a mono-nucleoside substance or a mono-nucleotide substance, as non-limiting examples) and one or more RNA substance, may also comprise adjusting the viscosity of the composition to a viscosity greater than about 100 centipoise (cP). As non-limiting examples, the viscosity of a composition may be adjusted to a viscosity greater than about 100 cP; or greater than about 500 cP; or greater than about 1,000 cP; or greater than about 2,000 cP; or greater than about 5,000 cP; or greater than about 10,000 cP; or greater than about 20,000 cP; or greater than about 50,000 cP; or greater than about 100,000 cP; or greater than about 200,000 cP; or greater than about 500,000 cP; or greater than about 1,000,000 cP.

In other non-limiting embodiments one or more method comprising at least one of producing or providing one or more RNA stabilizing composition (e.g. a composition that may have a selectively adjustable viscosity) comprising one or more RNA stabilizing substance (e.g. a mono-nucleoside substance or a mono-nucleotide substance, as non-limiting examples) and one or more RNA substance, may also comprise adjusting the viscosity of the composition to a viscosity greater than about 100 centipoise (cP) prior to at least one of packaging, shipping, or storage. As non-limiting examples, prior to packaging, shipping, or storage the viscosity of a composition may be adjusted to a viscosity greater than about 100 cP; or greater than about 500 cP; or greater than about 1,000 cP; or greater than about 2,000 cP; or greater than about 5,000 cP; or greater than about 10,000 cP; or greater than about 20,000 cP; or greater than about 50,000 cP; or greater than about 100,000 cP; or greater than about 200,000 cP; or greater than about 500,000 cP; or greater than about 1,000,000 cP.

In other non-limiting embodiments one or more method comprising at least one of producing or providing one or more RNA stabilizing composition (e.g. a composition that may have a selectively adjustable viscosity) comprising one or more RNA stabilizing substance (e.g. a mono-nucleoside substance or a mono-nucleotide substance, as non-limiting examples) and one or more RNA substance, may also comprise adjusting the viscosity of the composition to a viscosity less than about 100,000 centipoise (cP) prior to the intended use (such as administering a medicament, vaccine, or therapeutic agent, as non-limiting examples). As non-limiting examples, the viscosity of a composition may be adjusted to a viscosity less than about 100,000 cP prior to the intended use; or less than about 50,000 cP prior to the intended use; or less than about 10,000 cP prior to the intended use; or less than about 5,000 cP prior to the intended use; or less than about 2,000 cP prior to the intended use; or less than about 1,000 cP prior to the intended use; or less than about 500 cP prior to the intended use; or less than about 200 cP prior to the intended use; or less than about 100 cP prior to the intended use; or less than about 50 cP prior to the intended use.

Non-limiting embodiments of one or more methods may comprise producing an RNA stabilizing composition with a selectively adjustable viscosity by mixing one or more RNA stabilizing substance (such as a mono-nucleotide substance) with at least one RNA substance (such as an at least partially purified RNA substance) to produce an RNA stabilizing composition. A method may further comprise placing the composition into a chamber (such as a vial, syringe, or multi-compartment syringe) and then adding a substance to increase the viscosity of the composition to a selected value or range. As a non-limiting example, a method may comprise adding a buffer to reduce the pH to a selected value (such as a pH below 7 as a non-limiting example) or adding an inorganic cation to a selected concentration (such as 10 mM or greater as a non-limiting example). Following adjusting the viscosity, a method may further comprise sealing the chamber, such as with a resealable cover or hermetically sealing the chamber. The chamber containing the high viscosity RNA stabilizing composition may then be at least one of packaged, transported, shipped, or stored as described herein. As a non-limiting example, a chamber containing the high viscosity composition may be provided for one or more intended uses, such as administering a pharmaceutical composition. A method may further comprise mixing one or more diluent, such as to increase the pH to a selected value or reduce the concentration of an inorganic cation, to reduce the viscosity of the composition to a selected range or value prior to use.

A method may also comprise providing instructions for use or one or more additional components to reduce the viscosity of the composition prior to use. Instructions for use may comprise instructions for mixing one or more component provided, such as a diluent comprising water (e.g. a buffer or water), or other suitable information described herein.

As a non-limiting example, if the chamber is a multi-compartment syringe as described herein, then a method may comprise placing an RNA stabilizing composition in a first compartment of the syringe and adjusting the viscosity to a selected value or range and placing a second component, such as a diluent comprising water (e.g. a buffer or water), into a second compartment. The syringe may then be hermetically sealed and at least one of packaged, transported, shipped, or stored as described herein. Prior to use, a seal separating the two compartments may then be broken, mixing the two components and reducing the viscosity of the composition to a desired value prior to use, such as administering a medicament, vaccine, or therapeutic agent as a non-limiting example.

RNA stabilizing compositions may be used to produce one or more products or compositions comprising at least one RNA substance. As non-limiting examples, at least one RNA stabilizing composition of the present disclosure may be produced for later use. As a non-limiting example, an RNA stabilizing composition may be used to produce a pharmaceutical composition comprising at least one RNA substance for later use.

As non-limiting embodiments of the present disclosure, one or more methods may comprise at least one of producing or providing at least one or more RNA stabilizing composition comprising one or more RNA stabilizing substance and at least one RNA substance.

Non-limiting example methods for producing an RNA stabilizing composition may comprise one or more method steps described herein, wherein one or more steps may be combined to produce one or more RNA stabilizing composition described herein. Non-limiting example steps in a method may include at least one of producing or packaging an RNA stabilizing composition comprising at least one RNA substance for later use.

A non-limiting example of one or more method for producing an RNA stabilizing composition comprising at least one RNA substance and at least one RNA stabilizing substance (such as a mono-nucleoside substance or a mono-nucleotide substance) may comprise mixing, or otherwise combining, an RNA stabilizing substance with a polymeric RNA substance after the RNA substance has been at least partially purified, such as undergoing at least one purification step following synthesis. As a non-limiting example, a method for producing an RNA stabilizing composition may comprise adding one or more RNA stabilizing substance (such as one or more mono-nucleoside substance or one or more mono-nucleotide substance, as non-limiting examples) to a polymeric RNA substance after the polymeric RNA substance has been at least one of synthesized or at least partially purified, such as by undergoing at least one purification step described herein, to produce an RNA stabilizing composition.

Another non-limiting example of one or more method for producing an RNA stabilizing composition comprising at least one RNA substance and at least one RNA stabilizing substance may comprise mixing, or otherwise combining, a specified amount of one or more RNA stabilizing substance with a polymeric RNA substance (such as an at least partially purified polymeric RNA substance) to produce an RNA stabilizing composition comprising a specified amount of a selected RNA stabilizing substance. As a non-limiting example, a method for producing an RNA stabilizing composition may comprise mixing, or otherwise combining, a minimum amount (e.g. minimum weight) of an exogenous mono-nucleoside substance or exogenous mono-nucleotide substance with a polymeric RNA substance (such as an at least partially purified polymeric RNA substance) to produce an RNA stabilizing composition comprising a defined minimum amount (e.g. total weight) of all mono-nucleosides or all mono-nucleotides; such as mixing at least 1 mg of at least one exogenous mono-nucleotide substance with a polymeric RNA substance to produce an RNA stabilizing composition comprising at least 1 mg of mono-nucleotides, as a non-limiting example.

Additionally, non-limiting example methods for providing an RNA stabilizing composition may comprise one or more method steps described herein. As a non-limiting example, steps in a method may include at least one of providing or packaging an RNA stabilizing composition comprising at least one RNA substance for later use.

RNA stabilizing compositions described herein may be provided comprising at least one RNA substance and at least one RNA stabilizing substance (such as a mono-nucleoside substance or a mono-nucleotide substance). Additionally, RNA stabilizing compositions may be provided as one or more products or compositions comprising at least one RNA substance. As a non-limiting example, an RNA stabilizing composition may be provided as a pharmaceutical composition comprising at least one RNA substance for later use.

As a non-limiting example one or more method for providing an RNA stabilizing composition may comprise providing an RNA stabilizing composition comprising at least one RNA stabilizing substance (such as a mono-nucleoside substance or a mono-nucleotide substance) and at least one polymeric RNA substance, wherein the polymeric RNA substance has been at least partially purified, such as by undergoing at least one purification step following synthesis.

As another non-limiting example one or more method for providing an RNA stabilizing composition may comprise providing a composition with a specified amount of one or more RNA stabilizing substance. As a non-limiting example, a method for providing an RNA stabilizing composition may comprise providing a composition comprising a minimum total amount (e.g. minimum total weight) of mono-nucleosides or mono-nucleotides, such as providing an RNA stabilizing composition comprising at least 1 mg of mono-nucleosides or mono-nucleotides, as a non-limiting example.

In some embodiments of the present disclosure a method for producing an RNA stabilizing composition may comprise a method whereby one or more RNA stabilizing substance (such as one or more mono-nucleoside substance or one or more mono-nucleotide substance, as non-limiting examples) may be combined with (e.g. mixed with or otherwise combined with) one or more RNA substance, wherein an RNA stabilizing substance may be combined with an RNA substance after the RNA has been synthesized (such as chemical synthesis, cellular synthesis, or transcription, as non-limiting examples) or at least partially purified to produce a composition comprising one or more RNA stabilizing substance and one or more RNA substance. As a non-limiting example method, a method for producing an RNA stabilizing composition may comprise adding (e.g. mixing or otherwise combining) one or more RNA stabilizing substance (such as one or more mono-nucleoside substance or one or more mono-nucleotide substance, as non-limiting examples) to a composition comprising one or more RNA substance after said RNA substance has been synthesized or at least partially purified to produce a composition comprising one or more RNA stabilizing substance and one or more RNA substance. As a non-limiting example, an at least partially purified RNA or at least partially purified RNA substance may be an RNA that has undergone at least one or more purification step described herein. As a non-limiting example, an at least partially purified RNA or at least partially purified RNA substance may be an RNA that has undergone at least 1, or least 2, or at least 3 purification steps, wherein the steps may be the same or different.

Non-limiting embodiments of one or more methods may comprise producing an RNA stabilizing composition by mixing one or more RNA stabilizing substance (such as a mono-nucleotide substance) with an at least partially purified RNA substance (such as an RNA substance that has undergone at least one purification step) to produce an RNA stabilizing composition. A method may further comprise mixing a specified amount of one or more RNA stabilizing substance, such as adding a specified concentration or total weight of a mono-nucleotide substance, as a non-limiting example. A specified amount may be a minimum weight or concentration that may be mixed or added to a composition to produce an RNA stabilizing composition comprising a minimum total weight or concentration of one or more RNA stabilizing substance. Following producing the composition, a method may comprise placing the composition into a chamber (such as a vial, syringe, or multi-compartment syringe) and hermetically sealing the chamber. A method may also comprise, at least one of packaging, transporting, shipping, or storing the chamber containing the RNA stabilizing composition. As a non-limiting example, a method may comprise providing the chamber containing the RNA stabilizing composition for one or more uses, such as administering a vaccine, medicament, or therapeutic agent if the composition is a pharmaceutical composition, as a non-limiting example.

A method may also comprise providing instructions for use that may comprise instructions for storage or dosing or other suitable information, as described herein.

In some embodiments of the present disclosure a method for producing an RNA stabilizing composition may comprise a method whereby one or more RNA stabilizing substance (such as one or more mono-nucleoside substance or one or more mono-nucleotide substance, as non-limiting examples) may be combined with (e.g. mixed with or otherwise combined with) one or more polymeric RNA substance, wherein an RNA stabilizing substance may be combined with a polymeric RNA substance after the RNA has been synthesized (such as chemical synthesis, cellular synthesis, or transcription, as non-limiting examples) or at least partially purified to produce a composition comprising one or more RNA stabilizing substance and one or more polymeric RNA substance.

As a non-limiting example method, a method for producing an RNA stabilizing composition may comprise adding (e.g. mixing or otherwise combining) one or more RNA stabilizing substance (such as one or more mono-nucleoside substance or one or more mono-nucleotide substance, as non-limiting examples) to a composition comprising one or more polymeric RNA substance after said polymeric RNA substance has been synthesized or at least partially purified to produce a composition comprising one or more RNA stabilizing substance and one or more RNA substance.

In some embodiments of the present disclosure a method for producing an RNA stabilizing composition may comprise a method whereby one or more exogenous mono-nucleoside substance or one or more exogenous mono-nucleotide substance may be combined with (e.g. mixed with or otherwise combined with) one or more polymeric RNA substance, wherein an exogenous mono-nucleoside substance or an exogenous mono-nucleotide substance may be combined with a polymeric RNA substance after the RNA has been synthesized (such as chemical synthesis, cellular synthesis, or transcription, as non-limiting examples) or at least partially purified, to produce a composition comprising one or more RNA stabilizing substance and one or more polymeric RNA substance where the total amount (e.g. total weight) of all nucleosides or all nucleotides within the composition is increased compared to the total amount of nucleosides or nucleotides within the composition prior to the addition of an exogenous mono-nucleoside or exogenous mono-nucleotide.

As a non-limiting example method, a method for producing an RNA stabilizing composition may comprise adding (e.g. mixing or otherwise combining) one or more exogenous mono-nucleoside substance or one or more exogenous mono-nucleotide substance to a composition comprising one or more polymeric RNA substance after said polymeric RNA substance has been synthesized or at least partially purified to produce a composition comprising one or more mono-nucleoside substance or one or more mono-nucleotide substance and one or more RNA substance, wherein the total amount (e.g. total weight) of all nucleosides or all nucleotides within the composition is increased compared to the total amount of nucleosides or nucleotides within the composition prior to the addition of an exogenous mono-nucleoside or exogenous mono-nucleotide.

In some embodiments of the present disclosure a method for producing an RNA stabilizing composition may comprise a method whereby one or more mono-nucleoside substance or one or more mono-nucleotide substance may be combined with (e.g. mixed with or otherwise combined with) one or more polymeric RNA substance, wherein a mono-nucleoside substance or a mono-nucleotide substance may be combined with a polymeric RNA substance after the RNA has been synthesized (such as chemical synthesis, cellular synthesis, or transcription, as non-limiting examples) or at least partially purified, to produce a composition comprising one or more RNA stabilizing substance and one or more RNA substance where the total concentration of all mono-nucleosides or all mono-nucleotides in a composition may be at least 1 mg/mL or greater, or may be at least 2 mg/mL or greater, or may be at least 5 mg/mL or greater, or may be at least 10 mg/mL or greater, or may be at least 15 mg/mL or greater, or may be at least 20 mg/mL or greater, or may be at least 25 mg/mL or greater, or may be at least 30 mg/mL or greater, or may be at least 40 mg/ml or greater, or may be at least 50 mg/mL or greater, or may be at least 100 mg/mL or greater.

As a non-limiting example method, a method for producing an RNA stabilizing composition may comprise adding (e.g. mixing or otherwise combining) one or more mono-nucleoside substance or one or more mono-nucleotide substance to a composition comprising one or more polymeric RNA substance after said polymeric RNA substance has been synthesized or at least partially purified to produce a composition comprising one or more mono-nucleoside substance or one or more mono-nucleotide substance and one or more RNA substance, wherein the total concentration of all mono-nucleosides or all mono-nucleotides in a composition may be at least 1 mg/mL or greater.

In some embodiments of the present disclosure a method for producing an RNA stabilizing composition may comprise a method whereby one or more mono-nucleoside substance or one or more mono-nucleotide substance may be combined with (e.g. mixed with or otherwise combined with) one or more polymeric RNA substance, wherein a mono-nucleoside substance or a mono-nucleotide substance may be combined with a polymeric RNA substance after the RNA has been synthesized (such as chemical synthesis, cellular synthesis, or transcription, as non-limiting examples) or at least partially purified, to produce a composition comprising one or more RNA stabilizing substance and one or more RNA substance where the total amount (e.g. total weight) of all mono-nucleosides or all mono-nucleotides in a composition may be at least 25 μg or greater, or may be at least 50 μg or greater, or may be at least 100 μg or greater, or may be at least 250 μg or greater, or may be at least 500 μg or greater, or may be at least 1 mg or greater, or may be at least 2 mg or greater, or may be at least 5 mg or greater, or may be at least 10 mg or greater, or may be at least 25 mg or greater, or may be at least 50 mg or greater.

As a non-limiting example method, a method for producing an RNA stabilizing composition may comprise adding (e.g. mixing or otherwise combining) one or more mono-nucleoside substance or one or more mono-nucleotide substance to a composition comprising one or more polymeric RNA substance after said polymeric RNA substance has been synthesized or at least partially purified to produce a composition comprising one or more mono-nucleoside substance or one or more mono-nucleotide substance and one or more RNA substance, wherein the total amount (e.g. total weight) of all mono-nucleosides or all mono-nucleotides in a composition may be at least 1 mg or greater.

In some embodiments of the present disclosure a method for producing an RNA stabilizing composition may comprise a method whereby one or more mono-nucleoside substance or one or more mono-nucleotide substance may be combined with (e.g. mixed with or otherwise combined with) one or more polymeric RNA substance, wherein a mono-nucleoside substance or a mono-nucleotide substance may be combined with a polymeric RNA substance after the RNA has been synthesized (such as chemical synthesis, cellular synthesis, or transcription, as non-limiting examples) or at least partially purified, to produce a composition comprising one or more RNA stabilizing substance and one or more RNA substance where the polymeric RNA substance may comprise a polymer of at least 10 or more nucleotides, or at least 20 or more nucleotides, or at least 50 or more nucleotides, or at least 100 or more nucleotides, or at least 200 or more nucleotides, or at least 500 or more nucleotides, or at least 1,000 or more nucleotides, or at least 1,500 or more nucleotides, or at least 2,000 or more nucleotides.

As a non-limiting example method, a method for producing an RNA stabilizing composition may comprise adding (e.g. mixing or otherwise combining) one or more mono-nucleoside substance or one or more mono-nucleotide substance to a composition comprising one or more polymeric RNA substance after said polymeric RNA substance has been synthesized or at least partially purified to produce a composition comprising one or more mono-nucleoside substance or one or more mono-nucleotide substance and one or more RNA substance, wherein the polymeric RNA substance may comprise a polymer of at least 100 nucleotides.

In some embodiments of the present disclosure a method for producing an RNA stabilizing composition may comprise a method whereby one or more mono-nucleoside substance or one or more mono-nucleotide substance may be combined with (e.g. mixed with or otherwise combined with) one or more polymeric RNA substance, wherein a mono-nucleoside substance or a mono-nucleotide substance may be combined with a polymeric RNA substance after the RNA has been synthesized (such as chemical synthesis, cellular synthesis, or transcription, as non-limiting examples) or at least partially purified, to produce a composition comprising one or more RNA stabilizing substance and one or more RNA substance where the total weight percent of all mono-nucleosides or all mono-nucleotides in a composition may be at least 0.01% or greater, or may be at least 0.02% or greater, or may be at least 0.05% or greater, or may be at least 0.1% or greater, or may be at least 0.2% or greater, or may be at least 0.5% or greater, or may be at least 1.0% or greater, or may be at least 2.0% or greater, or may be at least 3.0% or greater, or may be at least 4.0% or greater, or may be at least 5.0% or greater.

As a non-limiting example method, a method for producing an RNA stabilizing composition may comprise adding (e.g. mixing or otherwise combining) one or more mono-nucleoside substance or one or more mono-nucleotide substance to a composition comprising one or more polymeric RNA substance after said polymeric RNA substance has been synthesized or at least partially purified to produce a composition comprising one or more mono-nucleoside substance or one or more mono-nucleotide substance and one or more RNA substance, wherein the total weight percent of all mono-nucleosides or all mono-nucleotides in a composition may be at least 0.5% or greater.

In some embodiments, one or more method described herein may be combined with one or more other methods described herein, such as one or more methods for producing or for providing one or more RNA stabilizing composition as non-limiting examples. As a non-limiting example, one or more method that may comprise combining (e.g. mixing or otherwise combining) one or more RNA stabilizing substance (such as one or more mono-nucleoside substance or one or more mono-nucleotide substance, as non-limiting examples) with one or more RNA substance to produce a composition comprising one or more RNA stabilizing substance and one or more RNA substance, may be combined with one or more other method described herein. As a non-limiting example, a method combining at least one RNA stabilizing substance with one or more RNA substance to produce a composition comprising at least one RNA stabilizing substance and one or more RNA substance may be combined with a method for providing an RNA stabilizing composition. As a non-limiting example, one or more methods for producing or for providing one more RNA stabilizing composition described herein may be combined with or also comprise one or more steps from one or more other methods such as providing instructions for use or providing one or more additional components (such as a diluent as a non-limiting example).

As a non-limiting example, a method comprising producing an RNA stabilizing composition that may comprise combining (e.g. mixing or otherwise combining) one or more RNA stabilizing substance (e.g. a mono-nucleoside substance or a mono-nucleotide substance, as non-limiting examples) with one or more RNA substance to produce a composition that may have a selectively adjustable viscosity (e.g. the viscosity may be increased or decreased by adjusting the pH of the composition or by increasing or decreasing the concentration of one or more inorganic cations, as non-limiting examples), and adjusting the viscosity of the composition to a viscosity greater than about 100,000 centipoise (cP) prior to packaging, shipping, or storage; may be combined with a method comprising providing an RNA stabilizing composition, that may have a selectively adjustable viscosity, where the viscosity of the composition may be adjusted to a viscosity less than about 10,000 centipoise (cP) prior to the intended use (such as administering a medicament, therapeutic agent, or vaccine, as non-limiting examples). As a non-limiting example, the above methods may also comprise introducing or placing a composition into a chamber or hermetically sealing a chamber prior to packaging, shipping, or storage; or providing the composition in a chamber (such as a hermetically sealed chamber) or providing instructions for use or providing one or more additional components, such as a diluent for decreasing the viscosity of the composition prior to use.

As another non-limiting example, a method comprising producing an RNA stabilizing composition that may comprise combining (e.g. mixing or otherwise combining) one or more RNA stabilizing substance (e.g. a mono-nucleoside substance or a mono-nucleotide substance, as non-limiting examples) with one or more RNA substance to produce a composition may have a selectively adjustable viscosity (e.g. the viscosity may be increased or decreased by adjusting the pH of the composition or by increasing or decreasing the concentration of one or more inorganic cations, as non-limiting examples), and adjusting the viscosity of the composition to a viscosity greater than about 100,000 centipoise (cP) prior to packaging, shipping, or storage; may be combined with a method that may comprise combining (e.g. mixing or otherwise combining) one or more mono-nucleoside substance or one or more mono-nucleotide substance with one or more polymeric RNA substance, wherein the mono-nucleoside substance or mono-nucleotide substance may be combined with the polymeric RNA substance after the RNA has been synthesized (such as chemical synthesis, cellular synthesis, or transcription, as non-limiting examples) or at least partially purified, to produce a composition comprising one or more RNA stabilizing substance and one or more RNA substance where the total concentration of all mono-nucleosides or all mono-nucleotides in a composition may be at least 1 mg/mL or greater. As a non-limiting example, the above methods may also comprise introducing or placing a composition into a chamber or hermetically sealing a chamber prior to packaging, shipping, or storage; or providing the composition in a chamber (such as in a hermetically sealed chamber) or providing instructions for use or providing one or more additional components, such as a diluent for decreasing the viscosity of the composition prior to use.

In some embodiments, one or more methods described herein may be used to produce a composition comprising one or more RNA stabilizing substance (such as one or more mono-nucleoside substance or one or more mono-nucleotide substance, as non-limiting examples) and one or more RNA substance, wherein an RNA stabilizing composition may also comprise one or more additional substances including one or more additional RNA stabilizing substances, additive substances, cellular uptake agents, inorganic cations (or salts thereof), or water, as non-limiting examples.

### Multi-Nucleotide Section

In some embodiments an RNA stabilizing substance may comprise a multi-nucleotide substance. As used herein a multi-nucleotide substance is comprised of at least two (2) but less than 21 mono-nucleotides described herein linked together via a phosphodiester bond, wherein the mono-nucleotides may be the same or different.

In some embodiments an RNA stabilizing substance may comprise a multi-nucleotide substance wherein a multi-nucleotide substance may comprise between 2-20 mono-nucleotides linked together, or between 2-15 mono-nucleotides linked together, or between 2-10 mono-nucleotides linked together, or between 2-5 mono-nucleotides linked together, wherein the mono-nucleotides may be the same or different.

In some embodiments a multi-nucleotide substance may comprise a single type of mono-nucleotide. In some embodiments a multi-nucleotide substance may comprise no more than two types of mono-nucleotides (e.g. up to 2 types). In some embodiments a multi-nucleotide substance may comprise no more than three types of mono-nucleotides (e.g. up to 3 types). In some embodiments a multi-nucleotide substance may comprise no more than between 1-3 types of mono-nucleotides (e.g. 1, 2, or 3 three types). In some embodiments a multi-nucleotide substance may comprise no more than between 1-2 types of mono-nucleotides (e.g. 1 or 2 types).

In some embodiments a multi-nucleotide substance may only comprise mono-nucleotides comprising a uracil nucleobase. In some embodiments a multi-nucleotide substance may only comprise mono-nucleotides comprising a guanosine nucleobase. In some embodiments a multi-nucleotide substance may only comprise mono-nucleotides comprising a uracil or a guanosine nucleobase. In some embodiments a multi-nucleotide substance may only comprise mono-nucleotides comprising an adenosine or uracil nucleobase. In some embodiments a multi-nucleotide substance may only comprise mono-nucleotides comprising an adenosine or thymine nucleobase. In some embodiments a multi-nucleotide substance may only comprise mono-nucleotides comprising a cytosine or guanosine nucleobase.

In some embodiments a multi-nucleotide substance may comprise one or more mono-nucleotides comprising one of the following nucleobases, wherein the nucleobase may be a modified or unmodified nucleobase: a uracil nucleobase, a cytosine nucleobase, an isocytosine nucleobase, a pseudocytosine nucleobase, a pseudoisocytosine nucleobase, a pseudouracil nucleobase, an orotate nucleobase, a guanine nucleobase, an isoguanine nucleobase, an adenine nucleobase, a hypoxanthine nucleobase, an isohypoxanthine nucleobase, a xanthine nucleobase, a pyridine-nucleobase, a 2-pyrimidinone nucleobase, or a 4-pyrimidinone nucleobase.

In some embodiments a multi-nucleotide substance may comprise one or more of the following mono-nucleotides, wherein the mono-nucleotide may be a modified or unmodified mono-nucleotide: a uridine mono-nucleotide, a cytidine mono-nucleotide, an isocytidine mono-nucleotide, a thymidine mono-nucleotide, a pseudocytidine mono-nucleotide, a pseudoisocytidine mono-nucleotide, a pseudouridine mono-nucleotide, an orotidine mono-nucleotide, a guanosine mono-nucleotide, an isoguanosine mono-nucleotide, an adenosine mono-nucleotide, an inosine mono-nucleotide, an isoinosine mono-nucleotide, or a xanthosine mono-nucleotide

In some embodiments a multi-nucleotide substance may comprise only one type, or only up to two types (e.g. 1-2 types), or only up to three types (e.g. 1-3 types), of mono-nucleotides comprising one of the following nucleobases, wherein the nucleobase may be a modified or unmodified nucleobase: a uracil nucleobase, a cytosine nucleobase, an isocytosine nucleobase, a pseudocytosine nucleobase, a pseudoisocytosine nucleobase, a pseudouracil nucleobase, an orotate nucleobase, a guanine nucleobase, an isoguanine nucleobase, an adenine nucleobase, a hypoxanthine nucleobase, an isohypoxanthine nucleobase, a xanthine nucleobase, a pyridine-nucleobase, a 2-pyrimidinone nucleobase, or a 4-pyrimidinone nucleobase.

In some embodiments a multi-nucleotide substance may comprise only one type, or only up to two types (e.g. 1-2 types), or only up to three types (e.g. 1-3 types), of the following mono-nucleotides, wherein the mono-nucleotide may be a modified or unmodified mono-nucleotide: a uridine mono-nucleotide, a cytidine mono-nucleotide, an isocytidine mono-nucleotide, a pseudocytidine mono-nucleotide, a pseudoisocytidine mono-nucleotide, a pseudouridine mono-nucleotide, an orotidine mono-nucleotide, a guanosine mono-nucleotide, an isoguanosine mono-nucleotide, an adenosine mono-nucleotide, an inosine mono-nucleotide, an isoinosine mono-nucleotide, or a xanthosine mono-nucleotide.

In some embodiments, one or more multi-nucleotide substance may be combined with one or more RNA substance to produce a composition comprising one or more multi-nucleotide substance and one or more RNA substance. In some embodiments, one or more multi-nucleotide substance may be combined with one or more RNA substance to produce a composition comprising one or more multi-nucleotide substance and one or more RNA substance, wherein the RNA substance may be a polymeric RNA. In some embodiments, one or more multi-nucleotide substance may be combined with one or more RNA substance to produce a composition comprising one or more multi-nucleotide substance and one or more RNA substance, wherein the RNA substance may be a polymeric RNA where the polymeric RNA may comprise a polymer of at least 50 nucleotides, or at least 100 nucleotides, or at least 200 nucleotides, or at least 500 nucleotides, or at least 1,000 nucleotides, or at least 1,500 nucleotides, or at least 2,000 nucleotides.

In some embodiments an RNA stabilizing substance comprising a multi-nucleotide substance may be used in or to produce one or more RNA stabilizing composition described herein.

In some embodiments a composition may comprise one or more multi-nucleotide substance and one or more RNA substance, wherein a multi-nucleotide substance may be an exogenous multi-nucleotide. As a non-limiting example, an exogenous multi-nucleotide may be a multi-nucleotide wherein the multi-nucleotide may be added to (e.g. mixed with or otherwise combined with) a polymeric RNA substance after the RNA has been synthesized (such as chemical synthesis, cellular synthesis, or transcription, as non-limiting examples) or at least partially purified, to produce a composition comprising one or more RNA stabilizing substance and one or more RNA substance where the total amount (e.g. total weight) of all nucleotides within the composition is increased compared to the total amount of nucleotides within the composition prior to the addition of an exogenous multi-nucleotide.

In some embodiments an RNA stabilizing substance comprising a multi-nucleotide substance may be used in or to produce one or more RNA stabilizing composition described herein, where the total amount (e.g. total weight) of all nucleotides in a composition may be at least 25 μg or greater, or may be at least 50 μg or greater, or may be at least 100 μg or greater, or may be at least 250 μg or greater, or may be at least 500 μg or greater, or may be at least 1 mg or greater, or may be at least 2 mg or greater, or may be at least 5 mg or greater, or may be at least 10 mg or greater, or may be at least 25 mg or greater, or may be at least 50 mg or greater.

In some embodiments an RNA stabilizing substance comprising a multi-nucleotide substance may be used in or to produce one or more RNA stabilizing composition described herein, where the total concentration of all nucleotides in a composition may be at least 10 μg/mL or greater, or may be at least 50 μg/mL or greater, or may be at least 100 μg/mL or greater, or may be at least 250 μg/mL or greater, or may be at least 500 μg/mL or greater, or may be at least 750 μg/mL or greater, or may be at least 1 mg/mL or greater, or may be at least 2 mg/mL or greater, or may be at least 5 mg/mL or greater, or may be at least 10 mg/mL or greater, or may be at least 20 mg/mL or greater, or may be at least 50 mg/mL or greater.

In some embodiments an RNA stabilizing substance comprising a multi-nucleotide substance may be used in or to produce one or more RNA stabilizing composition described herein, where the total weight percent of all nucleotides in a composition may be at least 0.01% or greater, or may be at least 0.02% or greater, or may be at least 0.05% or greater, or may be at least 0.1% or greater, or may be at least 0.2% or greater, or may be at least 0.5% or greater, or may be at least 1.0% or greater, or may be at least 2.0% or greater, or may be at least 3.0% or greater, or may be at least 4.0% or greater, or may be at least 5.0% or greater.

In some embodiments of the present disclosure a method for producing an RNA stabilizing composition may comprise a method whereby one or more RNA stabilizing substance (such as a multi-nucleotide substance, as a non-limiting example) may be combined with (e.g. mixed with or otherwise combined with) one or more polymeric RNA substance, wherein an RNA stabilizing substance may be combined with a polymeric RNA substance after the RNA has been synthesized (such as chemical synthesis, cellular synthesis, or transcription, as non-limiting examples) or at least partially purified to produce a composition comprising one or more RNA stabilizing substance and one or more polymeric RNA substance.

As a non-limiting example method, a method for producing an RNA stabilizing composition may comprise adding (e.g. mixing or otherwise combining) one or more RNA stabilizing substance (such as a multi-nucleotide substance, as a non-limiting example) to a composition comprising one or more polymeric RNA substance after said polymeric RNA substance has been synthesized or at least partially purified to produce a composition comprising one or more RNA stabilizing substance and one or more RNA substance.

In some embodiments of the present disclosure a method for producing an RNA stabilizing composition may comprise a method whereby one or more exogenous multi-nucleotide substance may be combined with (e.g. mixed with or otherwise combined with) one or more polymeric RNA substance, wherein an exogenous multi-nucleotide substance may be combined with a polymeric RNA substance after the RNA has been synthesized (such as chemical synthesis, cellular synthesis, or transcription, as non-limiting examples) or at least partially purified, to produce a composition comprising one or more RNA stabilizing substance and one or more polymeric RNA substance where the total amount (e.g. total weight) of all nucleotides within the composition is increased compared to the total amount of nucleotides within the composition prior to the addition of an exogenous multi-nucleotide substance.

As a non-limiting example method, a method for producing an RNA stabilizing composition may comprise adding (e.g. mixing or otherwise combining) one or more exogenous multi-nucleotide substance to a composition comprising one or more polymeric RNA substance after said polymeric RNA substance has been synthesized or at least partially purified to produce a composition comprising one or more multi-nucleotide substance and one or more RNA substance, wherein the total amount (e.g. total weight) of all nucleotides within the composition is increased compared to the total amount of nucleotides within the composition prior to the addition of an exogenous multi-nucleotide substance.

In some embodiments of the present disclosure a method for producing an RNA stabilizing composition may comprise a method whereby one or more multi-nucleotide substance may be combined with (e.g. mixed with or otherwise combined with) one or more polymeric RNA substance, wherein a multi-nucleotide substance may be combined with a polymeric RNA substance after the RNA has been synthesized (such as chemical synthesis, cellular synthesis, or transcription, as non-limiting examples) or at least partially purified, to produce a composition comprising one or more RNA stabilizing substance and one or more RNA substance where the total concentration of all nucleotides in a composition may be at least 10 μg/mL or greater, or may be at least 50 μg/mL or greater, or may be at least 100 μg/mL or greater, or may be at least 250 μg/mL or greater, or may be at least 500 μg/mL or greater, or may be at least 750 μg/mL or greater, or may be at least 1 mg/mL or greater, or may be at least 2 mg/mL or greater, or may be at least 5 mg/mL or greater, or may be at least 10 mg/mL or greater, or may be at least 20 mg/mL or greater, or may be at least 50 mg/mL or greater.

As a non-limiting example method, a method for producing an RNA stabilizing composition may comprise adding (e.g. mixing or otherwise combining) one or more multi-nucleotide substance to a composition comprising one or more polymeric RNA substance after said polymeric RNA substance has been synthesized or at least partially purified to produce a composition comprising one or more multi-nucleotide substance and one or more RNA substance, wherein the total concentration of all nucleotides in a composition may be at least 100 μg/mL or greater.

In some embodiments of the present disclosure a method for producing an RNA stabilizing composition may comprise a method whereby one or more multi-nucleotide substance may be combined with (e.g. mixed with or otherwise combined with) one or more polymeric RNA substance, wherein a multi-nucleotide substance may be combined with a polymeric RNA substance after the RNA has been synthesized (such as chemical synthesis, cellular synthesis, or transcription, as non-limiting examples) or at least partially purified, to produce a composition comprising one or more RNA stabilizing substance and one or more RNA substance where the total amount (e.g. total weight) of all nucleotides in a composition may be at least 25 μg or greater, or may be at least 50 μg or greater, or may be at least 100 μg or greater, or may be at least 250 μg or greater, or may be at least 500 μg or greater, or may be at least 1 mg or greater, or may be at least 2 mg or greater, or may be at least 5 mg or greater, or may be at least 10 mg or greater, or may be at least 25 mg or greater, or may be at least 50 mg or greater.

As a non-limiting example method, a method for producing an RNA stabilizing composition may comprise adding (e.g. mixing or otherwise combining) one or more multi-nucleotide substance to a composition comprising one or more polymeric RNA substance after said polymeric RNA substance has been synthesized or at least partially purified to produce a composition comprising one or more multi-nucleotide substance and one or more RNA substance, wherein the total amount (e.g. total weight) of all nucleotides in a composition may be at least 100 μg or greater.

In some embodiments of the present disclosure a method for producing an RNA stabilizing composition may comprise a method whereby one or more multi-nucleotide substance may be combined with (e.g. mixed with or otherwise combined with) one or more polymeric RNA substance, wherein a multi-nucleotide substance may be combined with a polymeric RNA substance after the RNA has been synthesized (such as chemical synthesis, cellular synthesis, or transcription, as non-limiting examples) or at least partially purified, to produce a composition comprising one or more RNA stabilizing substance and one or more RNA substance where the total weight percent of all nucleotides in a composition may be at least 0.01% or greater, or may be at least 0.02% or greater, or may be at least 0.05% or greater, or may be at least 0.1% or greater, or may be at least 0.2% or greater, or may be at least 0.5% or greater, or may be at least 1.0% or greater, or may be at least 2.0% or greater, or may be at least 3.0% or greater, or may be at least 4.0% or greater, or may be at least 5.0% or greater.

As a non-limiting example method, a method for producing an RNA stabilizing composition may comprise adding (e.g. mixing or otherwise combining) one or more multi-nucleotide substance to a composition comprising one or more polymeric RNA substance after said polymeric RNA substance has been synthesized or at least partially purified to produce a composition comprising one or more multi-nucleotide substance and one or more RNA substance, wherein the total weight percent of all nucleotides in a composition may be at least 0.5% or greater.

In some embodiments of the present disclosure a method for producing an RNA stabilizing composition may comprise a method whereby one or more multi-nucleotide substance may be combined with (e.g. mixed with or otherwise combined with) one or more polymeric RNA substance, wherein a multi-nucleotide substance may be combined with a polymeric RNA substance after the RNA has been synthesized (such as by chemical synthesis, cellular synthesis, or transcription, as non-limiting examples) or at least partially purified, to produce a composition comprising one or more RNA stabilizing substance and one or more RNA substance where the polymeric RNA substance may comprise a polymer of least 50 nucleotides, or at least 100 nucleotides, or at least 200 nucleotides, or at least 500 nucleotides, or at least 1,000 nucleotides, or at least 1,500 nucleotides, or at least 2,000 nucleotides.

As a non-limiting example method, a method for producing an RNA stabilizing composition may comprise adding (e.g. mixing or otherwise combining) one or more multi-nucleotide substance to a composition comprising one or more polymeric RNA substance after said polymeric RNA substance has been synthesized or at least partially purified to produce a composition comprising one or more multi-nucleotide substance and one or more RNA substance, wherein the polymeric RNA substance may comprise a polymer of at least 100 nucleotides.

In some embodiments, one or more method described herein may be combined with one or more other method described herein, such as one or more method for producing or for providing an RNA stabilizing composition as non-limiting examples. As a non-limiting example, one or more method that may comprise combining (e.g. mixing or otherwise combining) one or more multi-nucleotide substance with one or more RNA substance to produce a composition comprising one or more RNA stabilizing substance and one or more RNA substance, may be combined with one or more other method described herein.

As a non-limiting example, a method for producing an RNA stabilizing composition that may comprise adding (e.g. mixing or otherwise combining) one or more multi-nucleotide substance to a composition comprising one or more polymeric RNA substance after said polymeric RNA substance has been synthesized or at least partially purified to produce a composition comprising one or more multi-nucleotide substance and one or more RNA substance, wherein the polymeric RNA may be at least 100 nucleotides long; may be combined with a method that may comprise adding (e.g. mixing or otherwise combining) one or more multi-nucleotide substance to a composition comprising one or more polymeric RNA substance after said polymeric RNA substance has been synthesized or at least partially purified to produce a composition comprising one or more multi-nucleotide substance and one or more RNA substance, wherein the total concentration of all nucleotides in a composition may be at least 100 μg/mL or greater.

In some embodiments, one or more of these same methods described herein may be used to produce a composition or combination comprising one or more RNA stabilizing substance (such as one or more multi-nucleotide substance, as a non-limiting example) and one or more RNA substance, wherein an RNA stabilizing composition or combination may also comprise one or more additional substance including one or more additional RNA stabilizing substance, additive substance, inorganic cation (or salt thereof), cellular uptake agent, or water, as non-limiting examples.

### Substituted Hippuric Section

In some embodiments an RNA stabilizing substance may comprise a substituted hippuric substance. In some embodiments an RNA stabilizing substance may comprise a substituted hippuric substance that has the formula [Formula 3-A]:

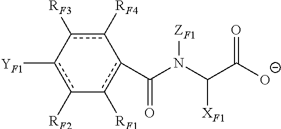

wherein:
[symbol] denotes a single or double bond;
R_{F1}-R_{F4} are each independent R_{F} groups and an R_{F} group is independently selected from hydrogen (H), hydroxy (—OH), oxo (═O), amino (—NH₂), methyl (—CH₃), methoxy (—O—CH₃), or ethoxy (—O—CH₂CH₃);
Y_{F1} is an independent Y_{F} group and a Y_{F} group is independently selected from hydrogen (H), hydroxy (—OH), oxo (═O), amino (—NH₂), methyl (—CH₃), methoxy (—O—CH₃), or ethoxy (—O—CH₂CH₃);
Z_{F1} is an independent Z_{F} group and a Z_{F} group is independently selected from hydrogen (H), methyl (—CH₃), or acetyl (—(C═O)—CH₃);
X_{F1} is an independent X_{F} group and an X_{F} group is independently selected from hydrogen (H), carboxylate (—COO⁻), amide ((—C═O)—NH₂), or the following:

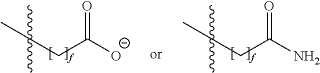

where f is an integer selected between 1-6.

In some embodiments an R_{F} group may be selected from hydrogen (H), hydroxy (—OH), oxo (═O), amino (—NH₂), methyl (—CH₃), methoxy (—O—CH₃), or ethoxy (—O—CH₂CH₃). In some embodiments an R_{F} group may be selected from hydrogen (H), hydroxy (—OH), oxo (═O), amino (—NH₂), methyl (—CH₃), or methoxy (—O—CH₃). In some embodiments an R_{F} group may be selected from hydrogen (H), hydroxy (—OH), amino (—NH₂), or methyl (—CH₃).

In some embodiments a Y_{F} group may be selected from hydrogen (H), hydroxy (—OH), oxo (═O), amino (—NH₂), methyl (—CH₃), methoxy (—O—CH₃), or ethoxy (—O—CH₂CH₃). In some embodiments a Y_{F} group may be selected from hydrogen (H), hydroxy (—OH), oxo (═O), amino (—NH₂), methyl (—CH₃), or methoxy (—O—CH₃). In some embodiments a Y_{F} group may be selected from hydrogen (H), hydroxy (—OH), amino (—NH₂), or methyl (—CH₃).

In some embodiments a Z_{F} group may be selected from hydrogen (H), methyl (—CH₃) or acetyl (—(C═O)CH₃). In some embodiments a Z_{F} group may be selected from hydrogen (H), or methyl (—CH₃).

In some embodiments an X_{F} group may be selected from hydrogen (H), carboxylate (—COO⁻), amide ((—C═O)—NH₂), (—([CH₂]_{f})—COO⁻), or (—([CH₂]_{f})—(C═O)—NH₂). In some embodiments an X_{F} group may be selected from hydrogen (H), carboxylate (—COO⁻) or (—([CH₂]_{f})—COO⁻). In some embodiments an X_{F} group may be selected from hydrogen (H) or (—([CH₂]_{f})—COO⁻). In some embodiments f may be an integer between 1-6, or between 1-4, or between 1-3, or between 1-2. In some embodiments f may be 1 or 2.

In some embodiments up to 3 R_{F} groups (e.g. 1, 2, or 3) may be hydroxy. In some embodiments up to 2 R_{F} groups (e.g. 1 or 2) may be oxo. In some embodiments up to 2 R_{F} groups (e.g. 1 or 2) may be amino. In some embodiments up to 2 R_{F} groups (e.g. 1 or 2) may be methyl. In some embodiments up to 2 R_{F} groups (e.g. 1 or 2) may be a methoxy. In some embodiments up to 2 R_{F} groups (e.g. 1 or 2) may be ethoxy.

In some embodiments Y_{F1} may be further selected from a substituted pteridine with the formula:

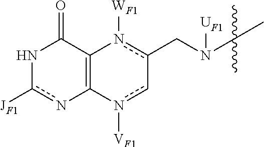

wherein:
[symbol] denotes a single or double bond;
J_{F1} is an independent J_{F} group and a J_{F} group is independently selected from hydrogen (H), hydroxy (—OH), oxo (═O), amino (—NH₂), or methyl (—CH₃);
W_{F1} is an independent W_{F} group and a W_{F} group is absent or independently selected from hydrogen (H), methyl (—CH₃), or formyl (—(CH)=O);
V_{F1} is an independent V_{F} group and a V_{F} group is absent or hydrogen (H);
U_{F1} is an independent U_{F} group and a U_{F} group is independently selected from hydrogen (H) or methyl (—CH₃).

In some embodiments a J_{F} group may be selected from hydrogen (H), hydroxy (—OH), oxo (═O), amino (—NH₂), or methyl (—CH₃). In some embodiments a J_{F} group may be selected from hydrogen (H), oxo (═O), or amino (—NH₂). In some embodiments a J_{F} group may be selected from oxo (═O) or amino (—NH₂). In some embodiments a J_{F} group may be amino (—NH₂).

In some embodiments a W_{F} group may be absent or selected from hydrogen (H), methyl (—CH₃), or formyl (—(CH)=O). In some embodiments a W_{F} group may be absent or selected from hydrogen (H) or methyl (—CH₃). In some embodiments a W_{F} group may be absent.

In some embodiments a U_{F} group may be selected from hydrogen (H) or methyl (—CH₃). In some embodiments a U_{F} group may be hydrogen (H).

In some embodiments an RNA stabilizing substance may comprise a substituted hippuric substance that has the formula [Formula 3-B]:

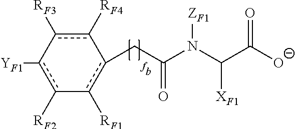

wherein:
[symbol] denotes a single or double bond;
R_{F1}-R_{F4} are each independent R_{F} groups as described in this section;
Y_{F1} is an independent Y_{F} group as described in this section;
Z_{F1} is an independent Z_{F} group as described in this section;
X_{F1} is an independent X_{F} group as described in this section;
f_{b} is an integer selected from 0-6.

In some embodiments f_{b} may be an integer between 1-6, or between 1-4, or between 1-3, or between 1-2. In some embodiments f_{b} may be 1 or 2.

A non-limiting example of a substituted hippuric substance of [Formula 3-A] is 4-hydroxyhippurate (also known as a conjugate base of 4-hydroxyhippuric acid) wherein R_{F1}-R_{F4} are H, Y_{F1} is hydroxy, Z_{F1} is H, and X_{F1} is H.

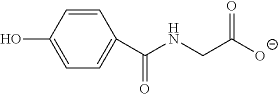 [SMILES: O=C([O-])CNC(=O)c1ccc(O)cc1]

A non-limiting example of a substituted hippuric substance of [Formula 3-A] is N-(4-aminobenzoyl)-L-glutamate (also known as a conjugate base of N-(4-aminobenzoyl)-L-glutamic acid) wherein R_{F1}-R_{F4} are H, Y_{F1} is amino, Z_{F1} is H, and X_{F1} is (—([CH₂]_{f})—COO⁻) and f is 2.

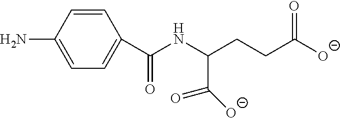 [SMILES: Nc1ccc(C(=O)NC(CCC(=O)[O-])C(=O)[O-])cc1]

A non-limiting example of a substituted hippuric substance of [Formula 3-A] is 5-methyltetrahydrofolate (also known as a conjugate base of 5-methyltetrahydrofolic acid or levomefolic acid) wherein R_{F1}-R_{F4} are H, Y_{F1} is a substituted pteridine where U_{F1} is H, W_{F1} is methyl, V_{F1} is H, and J_{F1} is amino, Z_{F1} is H, and X_{F1} is (—([CH₂]_{f})—COO⁻) and f is 2.

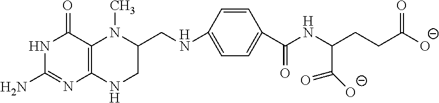 [SMILES: CN1c2c(nc(N)[nH]c2=O)NCC1CNc1ccc(C(=O)NC(CCC(=O)[O-])C(=O)[O-])cc1]

A non-limiting example of a substituted hippuric substance of [Formula 3-B] is phenylacetylglutamine (also known as phenylacetyl-L-glutamine) wherein R_{F1}-R_{F4} are H, Y_{F1} is H, Z_{F1} is H, f_{b} is 1, and X_{F1} is (—([CH₂]_{f})—(C═O)—NH₂)) and f is 2.

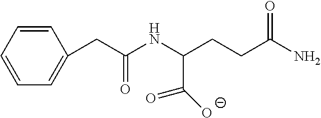 [SMILES: NC(=O)CCC(NC(=O)Cc1ccccc1)C(=O)[O-]]

A non-limiting example of a substituted hippuric substance of [Formula 3-B] is phenylacetylglycine wherein R_{F1}-R_{F4} are H, Y_{F1} is H, Z_{F1} is H, f_{b} is 1, and X_{F1} is H.

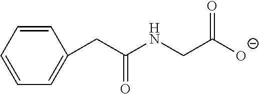 [SMILES: O=C([O-])CNC(=O)Cc1ccccc1]

Other non-limiting examples of substituted hippuric substances may include the following:

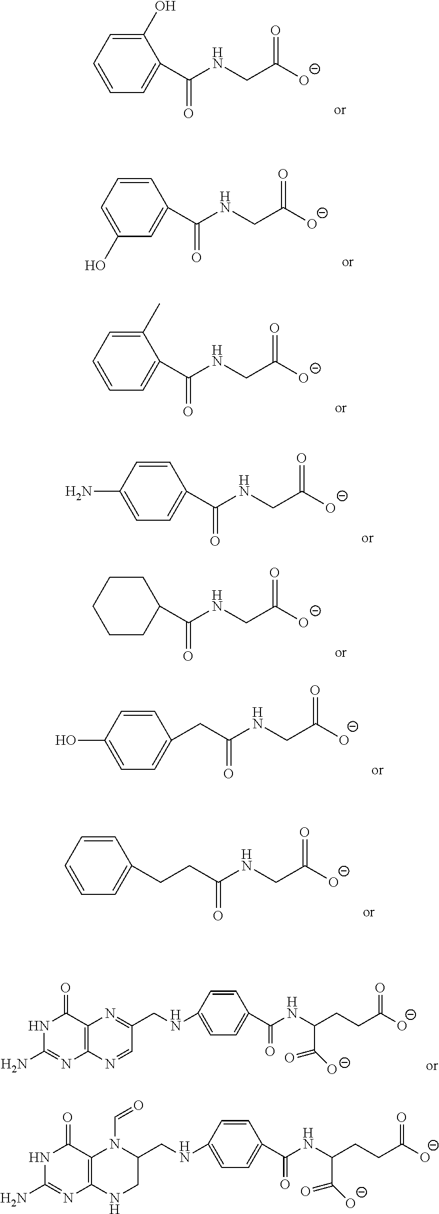

In some embodiments other substituted hippuric substances may include: N-(4-aminobenzoyl)-L-glutamate (also known as N-(4-aminobenzoyl)-L-glutamic acid), folate (also known as folic acid), 5-methyltetrahydrofolate (also known as 5-methyltetrahydrofolic acid, or levomefolic acid), folinate (also known as folinic acid, or 5-formyltetrahydrofolate), salicylurate (also known as salicyluric acid, or 2-hydroxyhippurate or 2-hydroxyhippuric acid), phenylacetyl-glutamine (including phenylacetyl-L-glutamine), phenylacetyl-glutamate (including phenylacetyl-L-glutamate), phenylacetylglycine, 4-hydroxyphenylacetylglycine, N-(3-phenylpropionyl)glycine, 4-hydroxyhippurate (also known as 4-hydroxyhippuric acid), 3-hydroxyhippurate (also known as 3-hydroxyhippuric acid), 4-aminohippurate (also known as 4-aminohippuric acid), 2-methylhippurate (also known as 2-methylhippuric acid), hexahydrohippurate (also known as hexahydrohippuric acid), folitixorin (also known as 5,10-methylenetetrahydrofolate), or 5,10-methenyltetrahydrofolate.

Embodiments comprising substituted hippuric substances, may include one or more conjugate acid or conjugate base, tautomer, stereoisomer, or salt thereof.

In some embodiments an RNA stabilizing substance comprising a substituted hippuric substance may be used in or to produce one or more RNA stabilizing composition described herein. In some embodiments an RNA stabilizing substance comprising a substituted hippuric substance may be used in or to produce one or more RNA stabilizing composition described herein, where the concentration of a substituted hippuric substance may be between about 5 mM-1M, or between about 10 mM-1M, or between about 20 mM-500 mM, as non-limiting examples (e.g. about 5 mM, 10 mM, 20 mM, 50 mM, 100 mM, 150 mM, 200 mM, 300 mM, 400 mM, 500 mM, or 1M, as non-limiting examples).

### Substituted Indole Section

In some embodiments an RNA stabilizing substance may comprise a substituted indole substance. In some embodiments an RNA stabilizing substance may comprise a substituted indole substance that has the formula [Formula 4-A1 or 4-A2]:

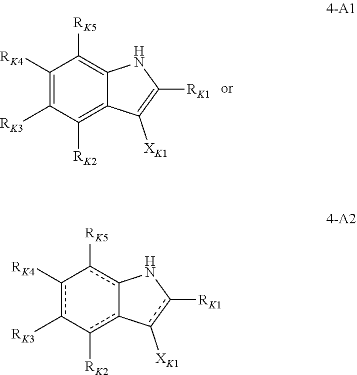

wherein:
[symbol] denotes a single or double bond;
R_{K1}—R_{K5} are each independent R_{K} groups and an R_{K} group is independently selected from hydrogen (H), hydroxy (—OH), oxo (═O), amino (—NH₂), methyl (—CH₃), methoxy (—O—CH₃), or carboxylate (—COO⁻);
X_{K1} is an independent Xx group and an Xx group is independently selected from hydrogen (H), hydroxy (—OH), oxo (═O), amino (—NH₂), methyl (—CH₃), methoxy (—O—CH₃), carboxylate (—COO⁻) or the following:

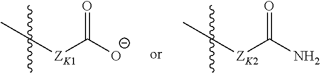

where Z_{K1} and Z_{K2} are independent Z_{K} groups and a Z_{K} group is independently selected from a C₁₋₄ alkyl group, that is optionally substituted with one or two of hydroxy (—OH), oxo (═O), or amino (—NH₂).

In some embodiments X_{K1} may be further selected from the following:

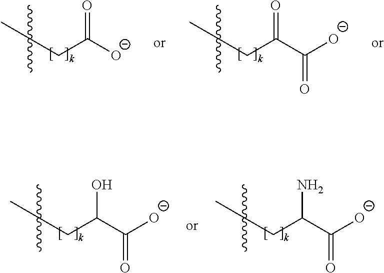

where k is an integer between 1-6.

In some embodiments k may be an integer between 1-6, or between 1-4, or between 1-3, or between 1-2. In some embodiments k may be 1 or 2.

In some embodiments an R_{K} group may be selected from hydrogen (H), hydroxy (—OH), oxo (═O), amino (—NH₂), methyl (—CH₃), methoxy (—O—CH₃), or carboxylate (—COO⁻). In some embodiments an R_{K} group may be selected from hydrogen (H), hydroxy (—OH), oxo (═O), amino (—NH₂), methyl (—CH₃), or carboxylate (—COO⁻). In some embodiments an R_{K} group may be selected from hydrogen (H), hydroxy (—OH), oxo (═O), or carboxylate (—COO⁻). In some embodiments an R_{K} group may be selected from hydrogen (H), hydroxy (—OH), or carboxylate (—COO⁻). In some embodiments an R_{K} group may be selected from hydrogen (H) or hydroxy (—OH).

In some embodiments an X_{K} group may be selected from hydrogen (H), hydroxy (—OH), oxo (═O), amino (—NH₂), methyl (—CH₃), methoxy (—O—CH₃), carboxylate (—COO⁻), —Z_{K1}—(COO⁻), —Z_{K2}—(C═O)—NH₂, —([CH₂]ₖ)—(COO⁻), —([CH₂]ₖ)—((CH)OH)—(COO⁻), —([CH₂]ₖ)—(C═O)—(COO⁻), or —([CH₂]ₖ)—((CH)NH₂)—(COO⁻). In some embodiments an X_{K} group may be selected from hydrogen (H), carboxylate (—COO⁻), —Z_{K1}—(COO⁻), —Z_{K2}—(C═O)—NH₂, —([CH₂]ₖ)—(COO⁻), —([CH₂]ₖ)—((CH)OH)—(COO⁻), —([CH₂]ₖ)—(C═O)—(COO⁻), or —([CH₂]ₖ)—((CH)NH₂)—(COO⁻). In some embodiments an X_{K} group may be selected from hydrogen (H), —Z_{K1}—(COO⁻), —([CH₂]ₖ)—(COO⁻), —([CH₂]ₖ)—((CH)OH)—(COO⁻), —([CH₂]ₖ)—(C═O)—(COO⁻), or —([CH₂]ₖ)—((CH)NH₂)—(COO⁻). In some embodiments an X group may be selected from hydrogen (H), —([CH₂]ₖ)—(COO⁻), —([CH₂]ₖ)—((CH)OH)—(COO⁻), —([CH₂]ₖ)—(C═O)—(COO⁻), or —([CH₂]ₖ)—((CH)NH₂)—(COO⁻).

In some embodiments up to 3 R_{K} groups (e.g. 1, 2, or 3) may be hydroxy. In some embodiments up to 2 R_{K} groups (e.g. 1 or 2) may be oxo. In some embodiments up to 2 R_{K} groups (e.g. 1 or 2) may be amino. In some embodiments up to 2 R_{K} groups (e.g. 1 or 2) may be methyl. In some embodiments up to 2 R_{K} groups (e.g. 1 or 2) may be a methoxy. In some embodiments up to 2 R_{K} groups (e.g. 1 or 2) may be ethoxy. In some embodiments up to 2 R_{K} groups (e.g. 1 or 2) may be carboxylate.

In some embodiments a Z_{K} group may be a C₁₋₄ alkyl group, that is optionally substituted with one or two of hydroxy (—OH), oxo (═O), or amino (—NH₂). In some embodiments a Z_{K} group may be a C₁₋₃ alkyl group, that is optionally substituted with one or two of hydroxy (—OH), oxo (═O), or amino (—NH₂). In some embodiments a Z_{K} group may be a C₁₋₂ alkyl group, that is optionally substituted with one of hydroxy (—OH), oxo (═O), or amino (—NH₂).

A non-limiting example of a substituted indole substance of [Formula 4-A] is indole-3-lactate (also known as a conjugate base of indole-3-lactic acid) wherein R_{K1}—R_{K5} are H, X_{K1} is —Z_{K1}—(COO⁻), and Z_{K1} is a C₂ alkyl substituted with one hydroxy.

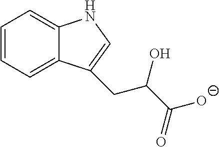 [SMILES: O=C([O-])C(O)Cc1c[nH]c2ccccc12]

A non-limiting example of a substituted indole substance of [Formula 4-A] is indole-3-pyruvate (also known as a conjugate base of indole-3-pyruvic acid) wherein R_{K1}—R_{K5} are H, X_{K1} is —Z_{K1}—(COO⁻), and Z_{K1} is a C₂ alkyl substituted with one oxo.

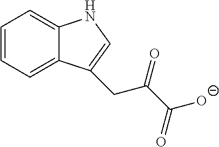 [SMILES: O=C([O-])C(=O)Cc1c[nH]c2ccccc12]

A non-limiting example of a substituted indole substance of [Formula 4-A] is 5-hydroxytryptophan (also known as 5-HTP) wherein R_{K1}, R_{K2}, R_{K4}, and R_{K5} are H, R_{K3} is hydroxy, X_{K1} is —([CH₂]ₖ)—((CH)NH₂)—(COO⁻), and k is 1.

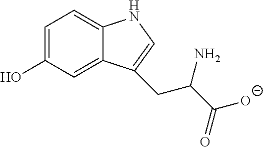 [SMILES: NC(Cc1c[nH]c2ccc(O)cc12)C(=O)[O-]]

Other non-limiting examples of substituted indole substances may include the following:

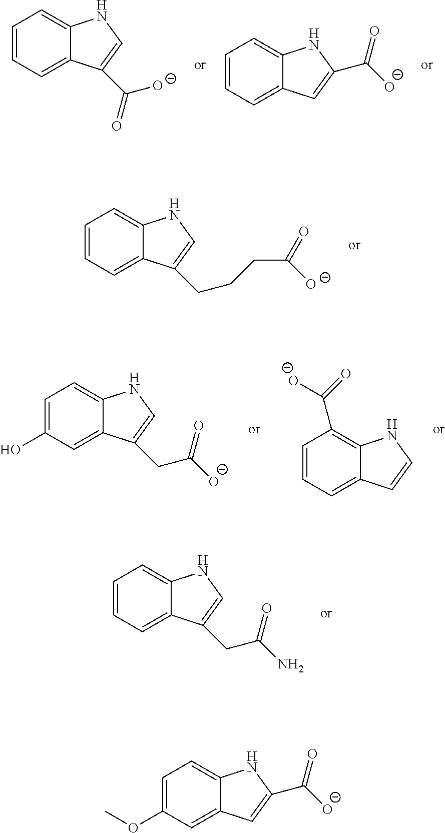

In some embodiments other substituted indole substances may include: indole-2-carboxylate, indole-3-carboxylate, indole-4-carboxylate, 5-hydroxy-indole-3-carboxylate, indoxyl-sulfate, indole-3-lactate, indole-3-butyrate, indole-3-acetate, 5-hydroxy-indole-3-acetate, indole-3-pyruvate, indole-3-acetamide, 5-hydroxy-tryptophan (including 5-hydroxy-L-tryptophan), indole-3-propionate, or 5-methoxy-indole-2-carboxylate.

Embodiments comprising substituted indole substances, may include one or more conjugate acid or conjugate base, tautomer, stereoisomer, or salt thereof.

In some embodiments an RNA stabilizing substance comprising a substituted indole substance may be used in or to produce one or more RNA stabilizing composition described herein. In some embodiments an RNA stabilizing substance comprising a substituted indole substance may be used in or to produce one or more RNA stabilizing composition described herein, where the concentration of a substituted indole substance may be between about 5 mM-1M, or between about 10 mM-1M, or between about 20 mM-500 mM, as non-limiting examples (e.g. about 5 mM, 10 mM, 20 mM, 50 mM, 100 mM, 150 mM, 200 mM, 300 mM, 500 mM, or 1M, as non-limiting examples).

### Acyclic Quaternary Ammonium and Tertiary Sulfonium Section

In some embodiments an RNA stabilizing substance may comprise an acyclic quaternary ammonium substance. In some embodiments an RNA stabilizing substance may comprise an acyclic tertiary sulfonium substance. In some embodiments an RNA stabilizing substance may comprise an acyclic quaternary ammonium substance or acyclic tertiary sulfonium substance that has the formula [Formula 5-A]:

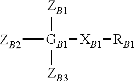

wherein:
G_{B1} is selected from nitrogen (N) or sulfur(S) and bonded to at least 3 carbon atoms;
Z_{B1}, Z_{B2}, and Z_{B3} are independent Z_{B} groups and a Z_{B} group is independently selected at each occurrence from a C₁₋₄ alkyl group, that is optionally substituted with one or two of hydroxy or oxo;

In some embodiments Z_{B3} may be absent;

If GB1 is sulfur, then Z_{B3} is absent;

X_{B1} is an independent X_{B} group and an X_{B} group is independently selected from a C₁₋₆ alkyl or alkenyl group, that is optionally substituted with one or two of hydroxy, oxo, acetoxy or amino;

R_{B1} is an independent RB group and an RB group is selected from the following:

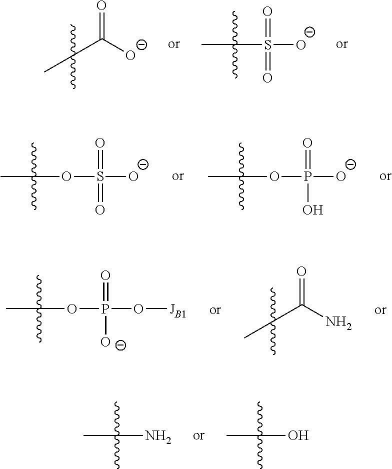

where (—COO⁻) is a carboxylate group, (—SO₃⁻) is a sulfonate group, (—O—SO₃⁻) is a sulfate group, (—O—PO₃⁻H) is a phosphate group, (—O—(PO₂⁻)—O-J_{B1}) is an organophosphate group, (—(C═O)—NH₂) is an amide group, (—NH₂) is an amino group, and (—OH) is a hydroxy group;

J_{B1} is an independent J_{B} group and a J_{B} group is independently selected from a C₁₋₆ alkyl group, that is optionally substituted with two, three, four, or five hydroxys.

In some embodiments R_{B1} may be selected from carboxylate (—COO⁻), sulfonate (—SO₃⁻), sulfate (—O—SO₃⁻), phosphate (—O—PO₃⁻H), organophosphate (—O—(PO₂⁻)—O-J_{B1}), or hydroxy (—OH). In some embodiments R_{B1} may be selected from carboxylate (—COO⁻), sulfonate (—SO₃⁻), phosphate (—O—PO₃⁻H), organophosphate (—O—(PO₂⁻)—O-J_{B1}), or hydroxy (—OH).

In some embodiments Z_{B1}, Z_{B2}, and Z_{B3} may be the same. In some embodiments at least two of Z_{B1}, Z_{B2}, and Z_{B3} may be the same. In some embodiments a Z_{B} group may be a methyl, ethyl, propyl, or butyl group, that is optionally substituted with one hydroxy. In some embodiments a Z_{B} group may be a C₁₋₄ alkyl group, that is optionally substituted with one hydroxy.

In some embodiments a Z_{B} group may be a C₁₋₂ alkyl group, that is optionally substituted with one hydroxy.

In some embodiments X_{B1} may be a C₁₋₆ alkyl group, that is optionally substituted with one or two of hydroxy or amino. In some embodiments X_{B1} may be a C₁₋₆ alkyl group, that is optionally substituted with one hydroxy. In some embodiments X_{B1} may be a C₁₋₄ alkyl group, that is optionally substituted with one hydroxy.

In some embodiments J_{B1} may be a C₁₋₃ alkyl group, that is optionally substituted with two hydroxys.

In some embodiments a Z_{B} group may be an alcohol group, such as a methanol, ethanol, butanol, or propanol group as non-limiting examples. In some embodiments Z_{B3} may be absent.

In some embodiments a Z_{B} group may comprise 1-4, 1-3, or 1-2 carbons (e.g. 1, 2, 3, or 4 carbons). In some embodiments a Z_{B} group may be a straight chain or branched. In some embodiments a Z_{B} group may comprise 1-2 hydroxy or oxo groups (e.g. 1 or 2 groups).

In some embodiments a J_{B} group may comprise 1-6, 1-4, or 1-3, carbons (e.g. 1, 2, 3, 4, 5, or 6 carbons). In some embodiments a J_{B} group may be a straight chain or branched. In some embodiments a J_{B} group may comprise 2-5 hydroxy groups (e.g. 2, 3, 4, or 5 groups).

In some embodiments an X_{B} group may comprise 1-6, 1-4, or 1-3, carbons (e.g. 1, 2, 3, 4, 5, or 6 carbons). In some embodiments an X_{B} group may be a straight chain or branched. In some embodiments an X_{B} group may comprise 1-2 hydroxy, amino, or oxo groups (e.g. 1 or 2 groups). In some embodiments an X_{B} group may be saturated, monounsaturated, or polyunsaturated.

In some embodiments a heteroatom is selected from N or O. In some embodiments a heteroatom is O.

### Acyclic Quaternary Ammonium

In some embodiments an RNA stabilizing substance may comprise an acyclic quaternary ammonium substance that has the formula [Formula 5-B]:

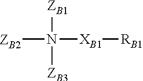

Where N is nitrogen;
Z_{B1}-Z_{B2} are independent Z_{B} groups described in this section;
Z_{B3} is absent or an independent Z_{B} group described in this section;
X_{B1} is an independent X_{B} group described in this section;
R_{B1} is an independent RB group described in this section.

A non-limiting example of an acyclic quaternary ammonium substance of [Formula 5-B] is choline, wherein Z_{B1}, Z_{B2}, and Z_{B3} are CH₃, X_{B1} is (CH₂)₂ and R_{B1} is (—OH)

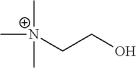

A non-limiting example of an acyclic quaternary ammonium substance of [Formula 5-B] is trimethylglycine (TMG) (also known as glycine betaine, or N,N,N-trimethylglycine), wherein Z_{B1}, Z_{B2}, and Z_{B3} are CH₃, X_{B1} is CH₂ and R_{B1} is (—COO⁻).

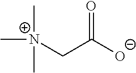

A non-limiting example of an acyclic quaternary ammonium substance of [Formula 5-B] is NDSB-195 (also known as dimethylethylammoniumpropane sulfonate or 3-[ethyl(dimethyl) ammonio]-1-propanesulfonate), wherein Z_{B1}, and Z_{B3} are CH₃, Z_{B2} is (CH₂)CH₃ and X_{B1} is (CH₂)₃ and R_{B1} is (—SO₃⁻).

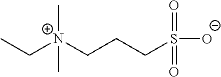

Other non-limiting examples of acyclic quaternary ammonium substances may include the following:

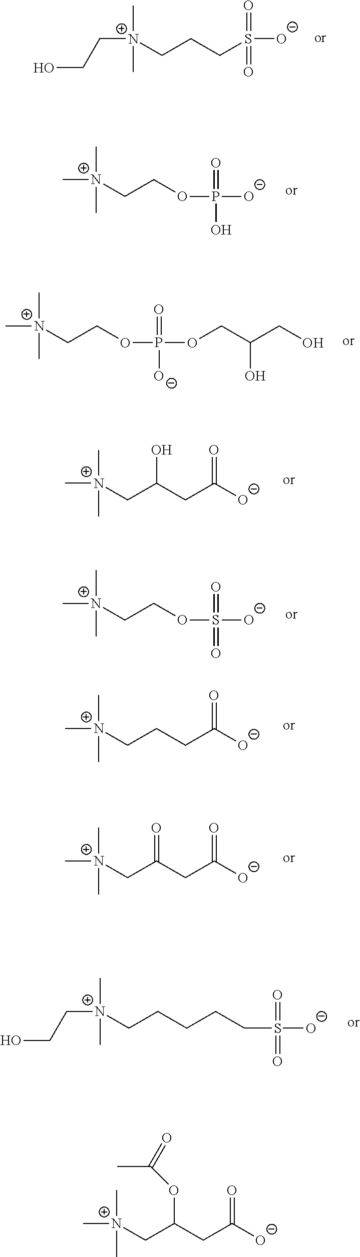

In some embodiments other acyclic quaternary ammonium substances may include: trimethylglycine, choline, carnitine, O-acetyl-carnitine, NDSB-195, 3-[trimethylammonio]-1-propanesulfonate, 3-[ethyl(dimethyl) ammonio]-1-butanesulfonate, 3-[diethyl(methyl) ammonio]-1-propanesulfonate, 3-[triethylammonio]-1-propanesulfonate, 3-[trimethylammonio]-1-butanesulfonate, 3-[dimethyl-(2-hydroxyethyl) ammonio]-1-propanesulfonate (NDSB-211), 3-[dimethyl-(2-hydroxyethyl) ammonio]-1-butanesulfonate, choline sulfate, phosphorylcholine, alpha-glycerophosphorylcholine (alpha-GPC), alanine betaine, beta-alanine betaine, N,N-dimethyl beta-alanine, N,N-dimethylglycine, gamma-butyrobetaine, valine betaine, N,N-dimethyl valine, Nε,Nε,Nε-trimethyllysine, and Nε, Nε-dimethyllysine.

Embodiments comprising acyclic quaternary ammonium substances, may include one or more conjugate acid or conjugate base, stereoisomer, or salt thereof. Stereoisomers may include D-isomers or L-isomers.

In some embodiments an RNA stabilizing substance comprising an acyclic quaternary ammonium substance may be used in or to produce one or more RNA stabilizing composition described herein. In some embodiments an RNA stabilizing substance comprising an acyclic quaternary ammonium substance may be used in or to produce one or more RNA stabilizing composition described herein, where the concentration of an acyclic quaternary ammonium substance may be between about 50 mM-5M, or between about 50 mM-3M, or between about 100 mM-2M, or between about 100 mM-1M, as non-limiting examples (e.g. about 50 mM, 100 mM, 200 mM, 400 mM, 500 mM, 600 mM, 800 mM, or 1M as non-limiting examples).

### Acyclic Tertiary Sulfonium

In some embodiments an RNA stabilizing substance may comprise an acyclic tertiary sulfonium substance that has the formula [Formula 5-C]:

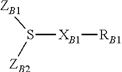

Where S is sulfur;
Z_{B1} and Z_{B2} are independent Z_{B} groups described in this section;
X_{B1} is an independent X_{B} group described in this section;
R_{B1} is an independent RB group described in this section.

In some embodiments Z_{B1} and Z_{B2} may be the same.

A non-limiting example of an acyclic tertiary sulfonium substance of [Formula 5-C] is dimethylsulfoniopropionate (DMSP), wherein Z_{B1} and Z_{B2} are CH₃, X_{B1} is (CH₂)₂ and R_{B1} is (—COO⁻)

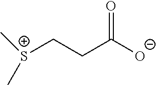

Other non-limiting examples of acyclic tertiary sulfonium substances may include the following:

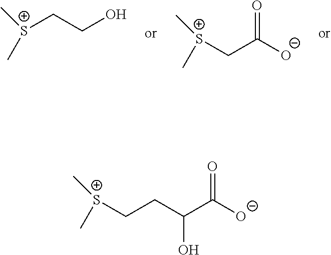

In some embodiments other acyclic tertiary sulfonium substances may include: dimethylsulfoniopropionate, S-methylmethionine, dimethylsulfonioacetate, diethylsulfoniopropionate, ethylmethylsulfoniopropionate, or methylpropylsulfoniopropionate.

In some embodiments an RNA stabilizing substance comprising an acyclic tertiary sulfonium substance may be used in or to produce one or more RNA stabilizing composition described herein. In some embodiments an RNA stabilizing substance comprising an acyclic tertiary sulfonium substance may be used in or to produce one or more RNA stabilizing composition described herein, where the concentration of an acyclic tertiary sulfonium substance may be between about 10 mM-3M, or between about 50 mM-2M, or between about 10 mM-1M, or between about 50 mM-1M, as non-limiting examples (e.g. about 10 mM, 50 mM, 100 mM, 200 mM, 400 mM, 500 mM, 600 mM, 800 mM, or 1M, as non-limiting examples).

### Substituted Piperidine and Substituted Morpholine Section

In some embodiments an RNA stabilizing substance may comprise a substituted piperidine substance. In some embodiments an RNA stabilizing substance may comprise a substituted morpholine substance. In some embodiments an RNA stabilizing substance may comprise a substituted piperidine or substituted morpholine substance that has the formula [Formula 6-A]:

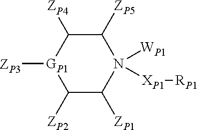

wherein:
N is a nitrogen bonded to at least 2 carbon atoms;
G_{P1} is selected from CH or oxygen (O);
Z_{P1}, Z_{P2}, Z_{P3}, Z_{P4}, and Z_{P5} are independent Z_{P} groups and a Z_{P} group is independently selected from hydrogen (H), hydroxy (—OH), oxo (═O), or carboxylate (—COO⁻);
W_{P1} is absent or selected from hydrogen (H) or a C₁₋₄ alkyl group, that is optionally substituted with one or two of hydroxy or oxo;
If G_{P1} is O, Z_{P3} is absent and W_{P1} is selected from a C₁₋₄ alkyl group, that is optionally substituted with one or two of hydroxy or oxo;
X_{P1} is absent or selected from a C₁₋₆ alkyl or alkenyl group, that is optionally substituted with one or two of hydroxy or oxo;
If X_{P1} is absent then R_{P1} is also absent;
If W_{P1} is hydrogen and X_{P1} is absent then at least one Z_{P} group is a carboxylate;
R_{P1} is absent or an independent R_{P} group and an R_{P} group is selected from hydrogen (H) or the following:

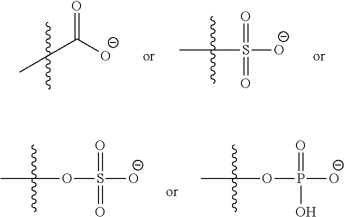

where (—COO⁻) is a carboxylate group, (—SO₃⁻) is a sulfonate group, (—O—SO₃⁻) is a sulfate group, and (—O—PO₃⁻H) is a phosphate group.

In some embodiments a Z_{P} group may be independently selected from hydrogen (H), hydroxy (—OH), oxo (═O), or carboxylate (—COO⁻). In some embodiments at least one Z_{P} group is a carboxylate. In some embodiments at least one Z_{P} group is a hydroxy or oxo. In some embodiments up to 2 Z_{P} groups may be a carboxylate, hydroxy, or oxo. In some embodiments up to 3 Z_{P} groups may be a carboxylate, hydroxy, or oxo.

In some embodiments W_{P1} may be absent. In some embodiments R_{P1} may be absent. In some embodiments X_{P1} may be absent.

In some embodiments R_{P1} may be selected from carboxylate (—COO⁻), sulfonate (—SO₃⁻), or phosphate (—O—PO₃⁻H). In some embodiments R_{P1} may be selected from carboxylate (—COO⁻) or sulfonate (—SO₃⁻). In some embodiments R_{P1} may be sulfonate (—SO₃⁻).

In some embodiments W_{P1} may be a C₁₋₄ alkyl group, that is optionally substituted with one of hydroxy or oxo. In some embodiments W_{P1} may be a C₁₋₃ alkyl group, that is optionally substituted with one of hydroxy or oxo. In some embodiments W_{P1} may be a methyl, ethyl, propyl, or butyl group.

In some embodiments X_{P1} may be a C₁₋₆ alkyl group, that is optionally substituted with one hydroxy. In some embodiments X_{P1} may be a C₁₋₄ alkyl group, that is optionally substituted with one hydroxy.

In some embodiments at least one of Z_{P1}-Z_{P5} may be a carboxylate. In some embodiments at least one of Z_{P1}-Z_{P5} may be a hydroxy or oxo. In some embodiments Z_{P3} may be absent.

In some embodiments W_{P1} may be a methyl, ethyl, propyl, or butyl, optionally substituted with 1 hydroxy group. In some embodiments W_{P1} may be an alcohol group, such as a methanol, ethanol, butanol, or propanol group as non-limiting examples.

In some embodiments W_{P1} may comprise 1-4, 1-3, or 1-2 carbons (e.g. 1, 2, 3, or 4 carbons). In some embodiments W_{P1} may be a straight chain or branched. In some embodiments W_{P1} may comprise 1-2 hydroxy groups or oxo groups (e.g. 1 or 2 groups).

In some embodiments X_{P1} may comprise 1-6, 1-4, or 1-3 carbons (e.g. 1, 2, 3, 4, 5, or 6 carbons). In some embodiments X_{P1} may be a straight chain or branched. In some embodiments X_{P1} may comprise 1-2 hydroxy or oxo groups (e.g. 1 or 2 groups). In some embodiments X_{P1} may saturated, monounsaturated, or polyunsaturated.

### Substituted Piperidine

In some embodiments an RNA stabilizing substance may comprise a substituted piperidine substance that has the formula [Formula 6-B]:

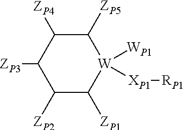

Where N is nitrogen;
Z_{P1}-Z_{P5} are independent Z_{P} groups described in this section;
W_{P1} is absent or selected from hydrogen (H) or a C₁₋₄ alkyl group, that is optionally substituted with one or two of hydroxy or oxo;
X_{P1} is absent or selected from a C₁₋₆ alkyl or alkenyl group, that is optionally substituted with one or two of hydroxy or oxo;
If X_{P1} is absent then R_{P1} is also absent;
If W_{P1} is hydrogen and X_{P1} is absent then at least one Z_{P} group is a carboxylate;
R_{P1} is absent or an independent R_{P} group described in this section.

A non-limiting example of a substituted piperidine substance of [Formula 6-B] is pipecolic acid betaine (also known as homostachydrine), wherein Z_{P1}-Z_{P4} are H, and Z_{P5} is a carboxylate, X_{P1} and W_{P1} are CH₃ and R_{P1} is absent.

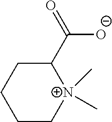

A non-limiting example of a substituted piperidine substance of [Formula 6-B] is mepiquat (also known as 1,1-dimethylpiperidinium or N,N-dimethylpiperidinium) wherein Z_{P1}-Z_{P5} are H, X_{P1} and W_{P1} are CH₃ and R_{P1} is absent

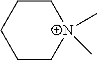

A non-limiting example of a substituted piperidine substance of [Formula 6-B] is 3-hydroxy-pipecolic acid (shown as 3-hydroxy-pipecolate; also known as 3-hydroxypiperidine-2-carboxylate) wherein Z_{P1}-Z_{P3} are H, Z_{P4} is hydroxy and Z_{P5} is carboxylate, W_{P1} is H, and X_{P1} and R_{P1} are absent.

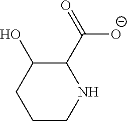

Other non-limiting examples of substituted piperidine substances may include the following:

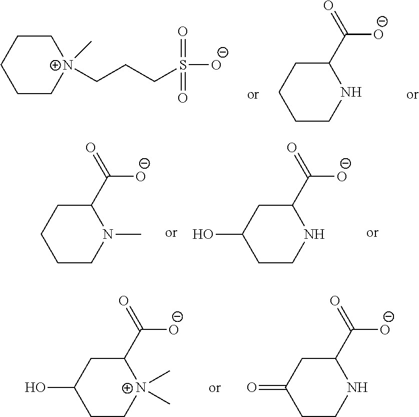

In some embodiments other substituted piperidine substances may include: pipecolic acid betaine, mepiquat, 1,1-diethylpiperidinium, 1-ethyl-1-methylpiperidinium, (3, 4, 5, or 6)-hydroxy-pipecolate, 3-(1-methylpiperidinio)-1-propanesulfonate (NDSB-221), pipecolate, (3, 4, 5, or 6)-oxo-pipecolate, (3, 4, 5, or 6)-hydroxy-pipecolic acid betaine, (3, 4, 5, or 6)-oxo-pipecolic acid betaine, or 3-(1-methylpiperidinio)-1-butanesulfonate.

Embodiments comprising substituted piperidine substances, may include one or more conjugate acid or conjugate base, stereoisomer, or salt thereof. Stereoisomers may include D-isomers or L-isomers.

In some embodiments an RNA stabilizing substance comprising a substituted piperidine substance may be used in or to produce one or more RNA stabilizing composition described herein. In some embodiments an RNA stabilizing substance comprising a substituted piperidine substance may be used in or to produce one or more RNA stabilizing composition described herein, where the concentration of a substituted piperidine substance may be between about 10 mM-3M, or between about 10 mM-2M, or between about 50 mM-2M, or between about 50 mM-1M, as non-limiting examples (e.g. about 50 mM, 100 mM, 200 mM, 400 mM, 500 mM, 600 mM, 800 mM, or 1M, as non-limiting examples).

### Substituted Morpholine

In some embodiments an RNA stabilizing substance may comprise a substituted morpholine substance that has the formula [Formula 6-C]:

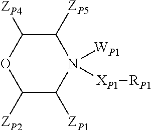

Where N is nitrogen and O is oxygen;
Z_{P1}, Z_{P2}, Z_{P4}, and Z_{P5} are independent Z_{P} groups described in this section;
W_{P1} is selected from hydrogen (H) or a C₁₋₄ alkyl group, that is optionally substituted with one or two of hydroxy or oxo;
X_{P1} is selected from a C₁₋₆ alkyl or alkenyl group, that is optionally substituted with one or two of hydroxy or oxo;
R_{P1} is absent or an independent R_{P} group described in this section.

A non-limiting example of a substituted morpholine substance of [Formula 6-C] is NDSB-223 (also known as, N-methyl-N-(3-sulfopropyl) morpholinium), wherein Z_{P1}, Z_{P2}, Z_{P4}, and Z_{P5} are H, W_{P1} is CH₃, X_{P1} is (CH₂)₃ and R_{P1} is sulfonate.

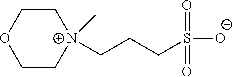

In some embodiments other substituted morpholine substances may include: NDSB-223, N-methyl-N-(3-sulfobutyl) morpholinium, N-ethyl-N-(3-sulfopropyl) morpholinium, or N-ethyl-N-(3-sulfobutyl) morpholinium.

Embodiments comprising substituted morpholine substances, may include one or more conjugate acid or conjugate base, or salt thereof.

In some embodiments an RNA stabilizing substance comprising a substituted morpholine substance may be used in or to produce one or more RNA stabilizing composition described herein.

In some embodiments an RNA stabilizing substance comprising a substituted morpholine substance may be used in or to produce one or more RNA stabilizing composition described herein, where the concentration of a substituted morpholine substance may be between about 10 mM-3M, or between about 10 mM-2M, or between about 50 mM-2M, or between about 50 mM-1M, as non-limiting examples (e.g. about 50 mM, 100 mM, 200 mM, 400 mM, 500 mM, 600 mM, 800 mM, or 1M, as non-limiting examples).

### Substituted Pyrrolidine Section

In some embodiments an RNA stabilizing substance may comprise a substituted pyrrolidine substance. In some embodiments an RNA stabilizing substance may comprise a substituted pyrrolidine substance that has the formula [Formula 7]:

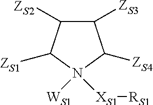

wherein:
N is a nitrogen bonded to at least 2 carbon atoms;
Z_{S1}, Z_{S2}, Z_{S3}, and Z_{S4} are independent Z_{S} groups and a Z_{S} group is independently selected from hydrogen (H), hydroxy (—OH), oxo (═O), or carboxylate (—COO⁻);
W_{S1} is absent or selected from hydrogen (H), or acetyl-(C═O)CH₃, or a C₁₋₄ alkyl group, that is optionally substituted with one or two of hydroxy or oxo;
X_{S1} is absent or selected from a C₁₋₆ alkyl or alkenyl group, that is optionally substituted with one or two of hydroxy or oxo;
If X_{S1} is absent then R_{S1} is absent;
Rₛ₁ is absent or selected from:

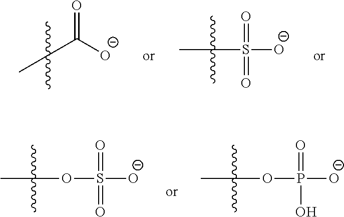

where (—COO⁻) is a carboxylate group, (—SO₃⁻) is a sulfonate group, (—O—SO₃⁻) is a sulfate group, and (—O—PO₃⁻H) is a phosphate group.

In some embodiments W_{S1}, X_{S1} or R_{S1} may be absent.

In some embodiments a Z_{S} group may be independently selected from hydrogen (H), hydroxy (—OH), oxo (═O), or carboxylate (—COO⁻). In some embodiments at least one Z_{S} group is a carboxylate. In some embodiments at least one Z_{S} group is a hydroxy or oxo. In some embodiments up to 2 Z_{S} groups may be a carboxylate, hydroxy, or oxo. In some embodiments up to 3 Z_{S} groups may be a carboxylate, hydroxy, or oxo.

In some embodiments R_{S1} may be selected from carboxylate (—COO⁻), sulfonate (—SO₃⁻), or phosphate (—O—PO₃⁻H). In some embodiments R_{S1} may be selected from carboxylate (—COO⁻) or sulfonate (—SO₃⁻). In some embodiments R_{S1} may be sulfonate (—SO₃⁻).

In some embodiments W_{S1} may be a C₁₋₄ alkyl group, that is optionally substituted with one hydroxy or one oxo. In some embodiments W_{S1} may be a C₁₋₃ alkyl group, that is optionally substituted with one hydroxy or one oxo. In some embodiments W_{S1} may be a methyl, ethyl, propyl, or butyl group.

In some embodiments X_{S1} may be a C₁₋₆ alkyl group, that is optionally substituted with one hydroxy or one oxo. In some embodiments X_{S1} may be a C₁₋₄ alkyl group, that is optionally substituted with one hydroxy or one oxo. In some embodiments X_{S1} may be a methyl, ethyl, propyl, or butyl group.

In some embodiments at least one of Z_{S1}-Z_{S4} may be a carboxylate. In some embodiments at least one of Z_{S1}-Z_{S4} may be a hydroxy or oxo.

In some embodiments W_{S1} may be a methyl, ethyl, propyl, or butyl, that is optionally substituted with one hydroxy or one oxo. In some embodiments a W_{S1} may be an alcohol group, such as a methanol, ethanol, butanol, or propanol group, as non-limiting examples.

In some embodiments W_{S1} may comprise 1-4, 1-3, or 1-2 carbons (e.g. 1, 2, 3, or 4 carbons). In some embodiments W_{S1} may be a straight chain or branched. In some embodiments W_{S1} may comprise 1-2 hydroxy groups or oxo groups (e.g. 1 or 2 groups).

In some embodiments X_{S1} may comprise 1-6, 1-4, or 1-3, carbons (e.g. 1, 2, 3, 4, 5, or 6 carbons). In some embodiments X_{S1} may be a straight chain or branched. In some embodiments X_{S1} may comprise 1-2 hydroxy groups or oxo groups (e.g. 1 or 2 groups). In some embodiments X_{S1} may saturated, monounsaturated, or polyunsaturated.

A non-limiting example of a substituted pyrrolidine substance of [Formula 7] is stachydrine (also known as proline betaine) wherein Z_{S1}-Z_{S3} are H, Z_{S4} is carboxylate, W_{S1} and X_{S1} are both CH₃, and R_{S1} is absent.

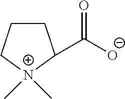

A non-limiting example of a substituted pyrrolidine substance of [Formula 7] is pyroglutamic acid (shown as a conjugate base (e.g. pyroglutamate), also known as 5-oxoproline) wherein Z_{S} is an oxo group, Z_{S2} and Z_{S3} are H, Z_{S4} is carboxylate, W_{S1} is H, and X_{S1} and R_{S1} are both absent.

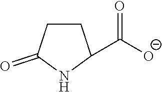

A non-limiting example of a substituted pyrrolidine substance of [Formula 7] is 1-butyl-1-methylpyrrolidinium wherein Z_{S1}-Z_{S4} are H, W_{S1} is CH₃, X_{S1} is a butyl group, and R_{S1} is absent.

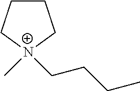

Other non-limiting examples of substituted pyrrolidine substances may include the following:

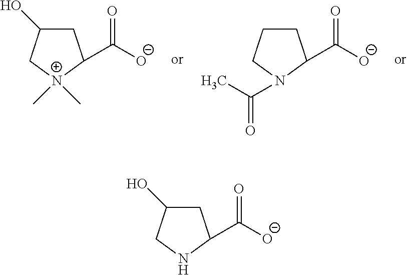

In some embodiments other substituted pyrrolidine substances may include: proline betaine, N-acetyl proline, 3-hydroxyproline, 4-hydroxyproline, 1-acetyl-3-hydroxyproline, 1-acetyl-4-hydroxyproline, 4-hydroxy-proline betaine, 4-oxoproline, 5-oxoproline (e.g pyroglutamate, including L-pyroglutamate), N-methyl proline, or N-methyl-4-hydroxy proline.

Embodiments comprising substituted pyrrolidine substances, may include one or more conjugate acid or conjugate base, stereoisomer, or salt thereof. Stereoisomers may include D-isomers or L-isomers.

In some embodiments an RNA stabilizing substance comprising a substituted pyrrolidine substance may be used in or to produce one or more RNA stabilizing composition described herein. In some embodiments an RNA stabilizing substance comprising a substituted pyrrolidine substance may be used in or to produce one or more RNA stabilizing composition described herein, where the concentration of a substituted pyrrolidine substance may be between about 50 mM-3M, or between about 50 mM-2M, or between about 50 mM-1M, as non-limiting examples (e.g. about 50 mM, 100 mM, 200 mM, 400 mM, 500 mM, 600 mM, 800 mM, or 1M, as non-limiting examples).

### Substituted Imidazole Section

In some embodiments an RNA stabilizing substance may comprise a substituted imidazole substance. In some embodiments an RNA stabilizing substance may comprise a substituted imidazole substance that has the formula [Formula 8]:

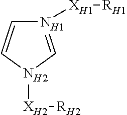

wherein:
N_{H1} and N_{H2} are each nitrogen (N) and each bonded to at least 2 carbon atoms;
X_{H1} is absent or an independent X_{H} group and an X_{H} group is independently selected from hydrogen (H) or a C₁₋₆ alkyl or alkenyl group or benzyl group, that is optionally substituted with one or two of hydroxy or oxo;
X_{H2} is an independent X_{H} group and an X_{H} group is independently selected from hydrogen (H) or a C₁₋₆ alkyl or alkenyl group or benzyl group, that is optionally substituted with one or two of hydroxy or oxo;
If X_{H1} is absent, then R_{H1} is also absent and X_{H2} is selected from a C₁₋₆ alkyl or alkenyl group, that is optionally substituted with one or two of hydroxy or oxo;
R_{H1} is absent or an independent RH group;
R_{H2} is absent or an independent RH group;
An RH group is independently selected from:

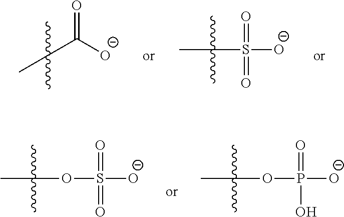

where (—COO⁻) is a carboxylate group, (—SO₃⁻) is a sulfonate group, (—O—SO₃⁻) is a sulfate group, and (—O—PO₃⁻H) is a phosphate group;

In some embodiments X_{H1} or R_{H1} may be absent.

In some embodiments an RH group may be selected from carboxylate (—COO⁻), sulfonate (—SO₃⁻), or phosphate (—O—PO₃⁻H). In some embodiments an RH group may be selected from carboxylate (—COO⁻) or sulfonate (—SO₃⁻). In some embodiments an RH group may be sulfonate (—SO₃⁻).

In some embodiments an X_{H} group may be a C₁₋₆ alkyl group, that is optionally substituted with one of hydroxy or oxo. In some embodiments an X_{H} group may be a C₁₋₄ alkyl group, that is optionally substituted with one of hydroxy or oxo. In some embodiments an X_{H} group may be a methyl, ethyl, propyl, butyl, or benzyl group, that is optionally substituted with one hydroxy

In some embodiments an X_{H} group may comprise 1-6, 1-4, or 1-3, carbons (e.g. 1, 2, 3, 4, 5, or 6 carbons). In some embodiments an X_{H} group may be a straight chain or branched. In some embodiments an X_{H} group may comprise 1-2 hydroxy or oxo groups (e.g. 1 or 2 groups). In some embodiments an X_{H} group may saturated, monounsaturated, or polyunsaturated.

A non-limiting example of a substituted imidazole substance of [Formula 8] may be 1-benzyl-3-methylimidazolium wherein R_{H1} and R_{H2} are absent, X_{H1} is CH₃, and X_{H2} is a benzyl group.

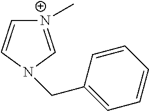

A non-limiting example of a substituted imidazole substance of [Formula 8] may be 1-butylsulfonate-3-methylimidazolium (also known as 1-methyl-3-(4-sulfobutyl) imidazolium) wherein R_{H1} is absent and R_{H2} is sulfonate, X_{H1} is CH₃, and X_{H2} is (CH₂)₄.

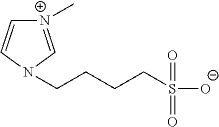

A non-limiting example of a substituted imidazole substance of [Formula 8] may be 1,3-bis(3-carboxypropyl)-1H-imidazole (also known as 1,3-bis(3-carboxypropyl)-1H-imidazolium) wherein R_{H1} and R_{H2} are both carboxylate and X_{H1} and X_{H2} are both (CH₂)₃.

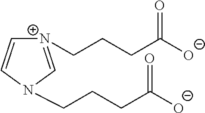 [SMILES: O=C([O-])CCCn1cc[n+](CCCC(=O)[O-])c1]

Other non-limiting examples of substituted imidazole substances may include the following:

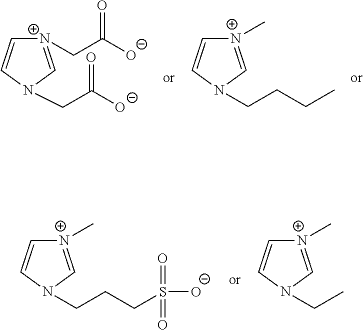

In some embodiments other substituted imidazole substances may include: 1-methyl-3-(4-sulfopropyl) imidazolium), 1-ethyl-3-(4-sulfobutyl) imidazolium), 1-ethyl-3-(4-sulfopropyl) imidazolium), 1-(2-hydroxyethyl) imidazole, 1,3-bis(3-carboxypropyl)-1H-imidazole, 1-butylsulfonate-3-methylimidazolium, 1-propylsulfonate-3-methylimidazolium, 1-benzyl-3-methylimidazolium, 1,3-Bis(carboxymethyl)-1H-imidazolium, 1,3-bis(carboxyethyl)-1H-imidazolium, 1-ethyl-3-methylimidazolium, 1-butyl-3-methylimidazolium, or 1-propyl-3-methylimidazolium.

Embodiments comprising substituted imidazole substances, may include one or more conjugate acid or conjugate base, or salt thereof.

In some embodiments an RNA stabilizing substance comprising a substituted imidazole substance may be used in or to produce one or more RNA stabilizing composition described herein.

In some embodiments an RNA stabilizing substance comprising a substituted imidazole substance may be used in or to produce one or more RNA stabilizing composition described herein, where the concentration of a substituted imidazole substance may be between about 10 mM-2M, or between about 50 mM-2M, or between about 50 mM-1M, as non-limiting examples (e.g. 50 mM, 100 mM, 200 mM, 400 mM, 500 mM, 600 mM, 800 mM, or 1M, as non-limiting examples).

### Substituted Benzene Section

In some embodiments an RNA stabilizing substance may comprise a substituted benzene substance. In some embodiments an RNA stabilizing substance may comprise a substituted benzene substance that has the formula [Formula 9]:

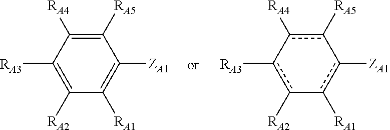

wherein:
[symbol] denotes a single or double bond;
R_{A1}—R_{A5} are each independent R_{A} groups and an R_{A} group is independently selected from hydrogen (H), hydroxy (—OH), oxo (═O), methyl (—CH₃), carboxylate (—COO⁻), methoxy (—O—CH₃), ethoxy (—OCH₂CH₃), or acetoxy (—O(C═O)CH₃); If an R group is oxo, then the adjacent [symbol] bond within the six membered ring is a single bond;
Z_{A1} is selected from:

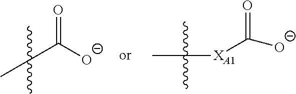

X_{A1} is selected from a C₁₋₂ alkyl or alkenyl group, that is optionally substituted with one hydroxy.

In some embodiments X_{A1} may be a C₁₋₂ alkyl, that is optionally substituted with one hydroxy. In some embodiments if an R_{A} group is a hydroxy, an R_{A} group may be etherified or esterified. In some embodiments if an R_{A} group is a hydroxy, an R_{A} group may be acetylated.

In some embodiments if Z_{A1} is a carboxylate, at least one R_{A} group is a carboxylate or hydroxy.

In some embodiments a Z_{A} group may be selected from carboxylate (—COO). In some embodiments an R_{A} group may be selected from hydrogen (H), hydroxy (—OH), methoxy (—O—CH₃), ethoxy (—OCH₂CH₃), or acetoxy (—O(C—O)CH₃). In some embodiments an R_{A} group may be selected from hydrogen (H), hydroxy (—OH), methoxy (—O—CH₃), or acetoxy (—O(C═O)CH₃). In some embodiments an R_{A} group may be selected from hydrogen (H), hydroxy (—OH), or carboxylate (—COO⁻). In some embodiments an R_{A} group may be selected from hydrogen (H), hydroxy (—OH), or acetoxy (—O(C═O)CH₃). In some embodiments an R_{A} group may be selected from hydrogen (H), or hydroxy (—OH).

In some embodiments up to 2 R_{A} groups (e.g. 1 or 2) may be a carboxylate. In some embodiments up to 2 or up to 3 R_{A} groups (e.g. 1, 2, or 3) may be a hydroxy. In some embodiments up to 2 R_{A} groups (e.g. 1 or 2) may be a methoxy or ethoxy. In some embodiments up to 2 R_{A} groups (e.g. 1 or 2) may be acetoxy. In some embodiments up to 5 R_{A} groups may be hydrogen (e.g. 1, 2, 3, 4, or 5).

In some embodiments a Z_{A} group or R_{A} group may comprise one or more conjugate acid or conjugate base or one or more protonated or deprotonated forms.

A non-limiting example of a substituted benzene substance of [Formula 9] is gallate (also known as 3,4,5-trihydroxybenzoate, or a conjugate base of gallic acid) wherein Z_{A1} is carboxylate, R_{A2}—R_{A4} are hydroxy (OH) and R_{A1} and RAS are hydrogen.

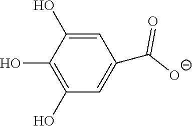 [SMILES: O=C([O-])c1cc(O)c(O)c(O)c1]

A non-limiting example of a substituted benzene substance of [Formula 9] is mandelate (also known as a conjugate base of mandelic acid), or 2-hydroxy-2-phenylacetate) wherein Z_{A1} is (X_{A1})—(COO⁻), X_{A1} is a C₁ alkyl that is substituted with one hydroxy group, and R_{A1}—R_{A5} are hydrogen.

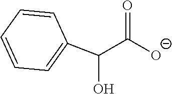 [SMILES: O=C([O-])C(O)c1ccccc1]

A non-limiting example of a substituted benzene substance of [Formula 9] is sinapinate (also known as a conjugate base of sinapinic acid) wherein Z_{A1} is (X_{A1})—(COO⁻), X_{A1} is a C₂ alkenyl, R_{A1} and RAS are hydrogen, R_{A2} and R_{A4} are methoxy (—OCH₃) and R_{A3} is hydroxy.

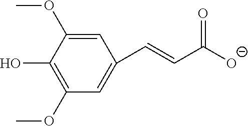 [SMILES: COc1cc(/C=C/C(=O)[O-])cc(OC)c1O]

Other non-limiting examples of substituted benzene substances may include the following:

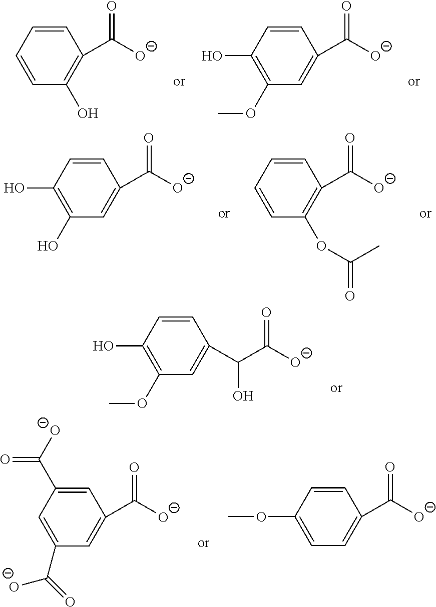

In some embodiments other substituted benzene substances may include: gallate, 3-hydroxybenzoate, 4-hydroxybenzoate, salicylate (also known as 2-hydroxybenzoate), 2-acetoxybenzoate, 1,2-benzenedicarboxylate, 1,3-benzenedicarboxylate, 1,4-benzenedicarboxylate, trimesate (e.g. a conjugate base of trimesic acid), benzene-1,3,5-tricarboxylate, cinnamate (e.g. a conjugate base of cinnamic acid), combinations of (2-5)-trimethoxybenzoate (e.g 3,4,5-trimethoxybenzoate or 2,4,5-trimethoxybenzoate or combinations thereof), 4-hydroxy-3-methoxybenzoate, vanillate (e.g. a conjugate base of vanillic acid), 4-hydroxy-3-methoxybenzoate, 5-hydroxy-3-methoxybenzoate, 4-hydroxy-3,5-dimethoxybenzoate, 2-hydroxy benzoate, 3-hydroxy benzoate, 4 hydroxy benzoate, or combinations of (2-6)-dihydroxy benzoate (e.g. 2,4-dihydroxybenzoate, 3,4-dihydroxybenzoate, or 2,6-dihydroxybenzoate, as non-limiting examples).

Embodiments comprising substituted benzene substances, may include one or more conjugate acid or conjugate base, tautomer, or salt thereof.

In some embodiments an RNA stabilizing substance comprising a substituted benzene substance may be used in or to produce one or more RNA stabilizing composition described herein. In some embodiments an RNA stabilizing substance comprising a substituted benzene substance may be used in or to produce one or more RNA stabilizing composition described herein, where the concentration of a substituted benzene substance may be between about 5 mM-1M, or between about 20 mM-1M, or between about 20 mM-500 mM, as non-limiting examples (e.g. about 20 mM, 50 mM, 100 mM, 150 mM, 200 mM, 300 mM, or 500 mM, as non-limiting examples).

### Substituted Pyridine Section

In some embodiments an RNA stabilizing substance may comprise a substituted pyridine substance. In some embodiments an RNA stabilizing substance may comprise a substituted pyridine substance that has the formula [Formula 10]:

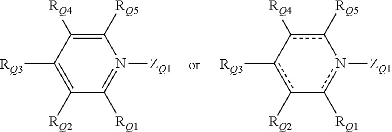

wherein:
[symbol] denotes a single or double bond;
R_{Q1}—R_{Q5} are each independent R_{Q} groups and an R_{Q} group is independently selected from hydrogen (H), hydroxy (—OH), oxo (═O), methyl (—CH₃), carboxylate (—COO⁻), amide ((—C═O)—NH₂), amino (—NH₂), methoxy (—O—CH₃), ethoxy (—OCH₂CH₃), or acetoxy (—O(C═O)CH₃);
If an R group is oxo, then the adjacent [symbol] bond within the six membered ring is a single bond;
Z_{Q1} is absent or selected from the following:

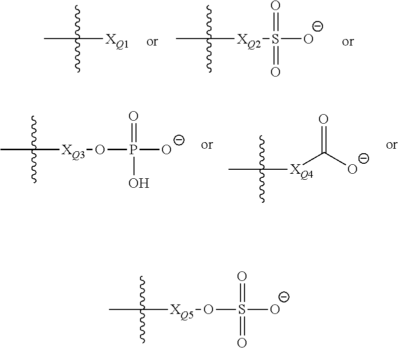

where X_{Q1}—X_{Q5} are independent X_{Q} groups and an X_{Q} group is independently selected from a C₁₋₆ alkyl or alkenyl group, that is optionally substituted with one or two of hydroxy or oxo;
If Z_{Q1} is absent then at least one R_{Q} group is a carboxylate (—COO⁻);

If an R_{Q} group is an amide, then a hydrogen on the amide nitrogen may be substituted with a carboxymethyl group (—CH₂)—(COO⁻).

In some embodiments Z_{Q1} is further selected from a methyl group (—CH₃) or an oxide (—O—).

In some embodiments if an R_{Q} group is a hydroxy, an R_{Q} group may be etherified or esterified. In some embodiments if an R_{Q} group is a hydroxy, an R_{Q} group may be acetylated

In some embodiments if an R_{Q} group is an amide, then a hydrogen on the amide nitrogen may be substituted with a carboxymethyl group (—CH₂)—(COO⁻) (e.g. an amide group becomes (—C—O)—(NH)—(CH₂)—(COO⁻)).

In some embodiments Z_{Q1} may be selected from —X_{Q1}, (—X_{Q2})—(SO₃⁻), (—X_{Q3})—(O—PO₃⁻H), or (—X_{Q4})—(COO⁻). In some embodiments Z_{Q1} may be selected from —X_{Q1}, (—X_{Q2})—(SO₃⁻), or (—X_{Q4})—(COO⁻). In some embodiments Z_{Q1} may be selected from —X_{Q1} or (—X_{Q2})—(SO₃⁻).

In some embodiments an R_{Q} group may be selected from hydrogen (H), hydroxy (—OH), carboxylate (—COO⁻), amide ((—C═O)—NH₂), amino (—NH₂), methoxy (—O—CH₃), ethoxy (—OCH₂CH₃), or acetoxy (—O(C═O)CH₃). In some embodiments an R_{Q} group may be selected from hydrogen (H), hydroxy (—OH), carboxylate (—COO⁻), amide ((—C═O)—NH₂)), methoxy (—O—CH₃), or acetoxy (—O(C═O)CH₃). In some embodiments an R_{Q} group may be selected from hydrogen (H), hydroxy (—OH), carboxylate (—COO⁻), methoxy (—O—CH₃), or acetoxy (—O(C═O)CH₃). In some embodiments an R_{Q} group may be selected from hydrogen (H), carboxylate (—COO⁻), or hydroxy (—OH). In some embodiments an R_{Q} group may be selected from hydrogen (H) or carboxylate (—COO⁻).

In some embodiments up to 2 R_{Q} groups (e.g. 1 or 2) may be methoxy (—O—CH₃) or ethoxy (—OCH₂CH₃). In some embodiments up to 2 R_{Q} groups (e.g. 1 or 2) may be acetoxy (—O(C═O)CH₃).

In some embodiments up to 2 R_{Q} groups or up to 3 R_{Q} groups (e.g. 1, 2, or 3) may be a carboxylate. In some embodiments up to 2 or up to 3 R_{Q} groups (e.g. 1, 2, or 3) may be a hydroxy.

In some embodiments up to 2 R_{Q} groups (e.g. 1 or 2) may be an amide. In some embodiments up to 2 R_{Q} groups (e.g. 1 or 2) may be an amide wherein a hydrogen on the amide nitrogen is substituted with a carboxymethyl group (—CH₂)—(COO⁻). In some embodiments up to 2 R_{Q} groups (e.g. 1 or 2) may be an amino. In some embodiments up to 5 R_{Q} groups may be hydrogen (e.g. 1, 2, 3, 4, or 5).

In some embodiments an X_{Q} group may be a C₁₋₆ alkyl group, that is optionally substituted with one of hydroxy or oxo. In some embodiments an X_{Q} group may be a C₁₋₄ alkyl group, that is optionally substituted with one of hydroxy or oxo.

In some embodiments an X_{Q} group may comprise 1-6, 1-4, or 1-3, carbons (e.g. 1, 2, 3, 4, 5, or 6 carbons). In some embodiments an X_{Q} group may be a straight chain or branched. In some embodiments an X_{Q} group may comprise 1-2 hydroxy or oxo groups (e.g. 1 or 2 groups). In some embodiments an X_{Q} group may be saturated, monounsaturated, or polyunsaturated.

In some embodiments a Z_{Q} group or R_{Q} group may comprise one or more conjugate acid or conjugate base or one or more protonated or deprotonated forms.

A non-limiting example of a substituted pyridine substance of [Formula 10] is quinolinate (also known 2,3-pyridine dicarboxylate, pyridine-2,3-dicarboxylate, or a conjugate base of quinolinic acid) wherein Z_{Q1} is absent, R_{Q1} and R_{Q2} are carboxylate, and R_{Q3}—R_{Q5} are hydrogen.

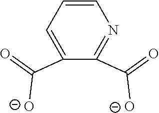 [SMILES: O=C([O-])c1cccnc1C(=O)[O-]]

A non-limiting example of a substituted pyridine substance of [Formula 10] is 1-methylnicotinamide (MNA) wherein Z_{Q1} is CH₃, R_{Q4} is amide, and R_{Q1}—R_{Q3} are hydrogen and R_{Q5} is hydrogen.

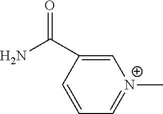

A non-limiting example of a substituted pyridine substance of [Formula 10] is NDSB-201 (also known as 3-(1-pyridinio)-1-propanesulfonate) wherein Z_{Q1} is (X_{Q2})—(SO₃—), and X_{Q2} is (CH₂)₃, and R_{Q1}—R_{Q5} are hydrogen.

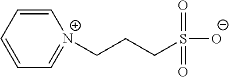 [SMILES: O=S(=O)([O-])CCC[n+]1ccccc1]

Other non-limiting examples of substituted pyridine substances may include the following:

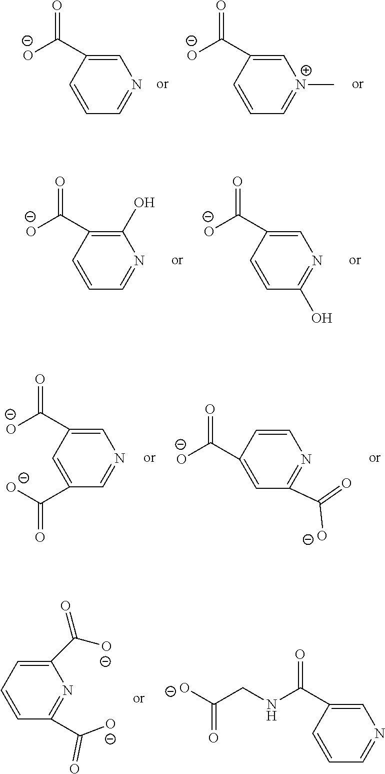

In some embodiments a substituted pyridine substance may be a pyridine dicarboxylate, wherein a pyridine dicarboxylate may include: pyridine-2,3-dicarboxylate, pyridine-2,4-dicarboxylate, pyridine-2,5-dicarboxylate, pyridine-2,6-dicarboxylate, pyridine-3,4-dicarboxylate, pyridine-3,5-dicarboxylate, or 4-hydroxy-pyridine-2,6-dicarboxylate.

In some embodiments a substituted pyridine substance may be a pyridine carboxylate, wherein a pyridine carboxylate may include: pyridine-3-carboxylate, pyridine-2-carboxylate, pyridine-4-carboxylate, 6-hydroxypyridine-2-carboxylate, 5-hydroxypyridine-2-carboxylate, 4-hydroxypyridine-2-carboxylate, 3-hydroxypyridine-2-carboxylate, 2-hydroxypyridine-3-carboxylate, 4-hydroxypyridine-3-carboxylate, 5-hydroxypyridine-3-carboxylate, 6-hydroxypyridine-3-carboxylate, 2-hydroxypyridine-4-carboxylate, or 3-hydroxypyridine-4-carboxylate.

In some embodiments other substituted pyridine substances may include: pyridine-2,3-dicarboxylate, pyridine-2,4-dicarboxylate, pyridine-2,5-dicarboxylate, pyridine-2,6-dicarboxylate, pyridine-3,4-dicarboxylate, pyridine-3,5-dicarboxylate, dinicotinate (also known as a conjugate base of dinicotinic acid), dipicolinate (also known as a conjugate base of dipicolinic acid), trigonelline (also known as 1-methylpyridinium-3-carboxylate), quinolinate, NDSB-201, 1-methylnicotinamide (MNA), nicotinamide N-oxide (NAO), nicotinurate (also known as a conjugate base of nicotinuric acid), nicotinate (also known as pyridine-3-carboxylate), pyridine-2-carboxylate, pyridine-4-carboxylate, nicotinamide (also known as pyridine-3-carboxamide), pyridine-4-carboxamide, pyridine-2-carboxamide, 6-hydroxypyridine-2-carboxylate, 5-hydroxypyridine-2-carboxylate, 4-hydroxypyridine-2-carboxylate, 3-hydroxypyridine-2-carboxylate, 2-hydroxypyridine-3-carboxylate, 4-hydroxypyridine-3-carboxylate, 5-hydroxypyridine-3-carboxylate, 6-hydroxypyridine-3-carboxylate, 2-hydroxypyridine-4-carboxylate, 3-hydroxypyridine-4-carboxylate, 4-hydroxy-pyridine-2,6-dicarboxylate, nicotinamide mononucleotide (NMN) (also known as 3-(aminocarbonyl)-1-(5-O-phosphono-beta-D-ribofuranosyl)-pyridinium), nicotinamide riboside (also known as 1-(beta-D-ribofuranosyl) nicotinamide), nicotinate mononucleotide (also known, nicotinate ribonucleoside 5′-phosphate, or 3-carboxy-1-(5-O-phosphono-beta-D-ribofuranosyl)-pyridinium), nicotinate riboside (also known as 1-(beta-D-ribofuranosyl) nicotinate), or 3-(1-pyridinio)-1-butylsulfonate).

Embodiments comprising substituted pyridine substances, may include one or more conjugate acid or conjugate base, tautomer, or salt thereof.

In some embodiments an RNA stabilizing substance comprising a substituted pyridine substance may be used in or to produce one or more RNA stabilizing composition described herein. In some embodiments an RNA stabilizing substance comprising a substituted pyridine substance may be used in or to produce one or more RNA stabilizing composition described herein, where the concentration of a substituted pyridine substance may be between about 5 mM-2M, or between about 20 mM-2M, or between about 20 mM-1M, as non-limiting examples (e.g. about 20 mM, 50 mM, 100 mM, 150 mM, 200 mM, 300 mM, 500 mM, or 1M, as non-limiting examples).

### Diazine Carboxylate Section

In some embodiments an RNA stabilizing substance may comprise a diazine carboxylate substance. In some embodiments an RNA stabilizing substance may comprise a diazine carboxylate substance that has the formula [Formula 11-A]:

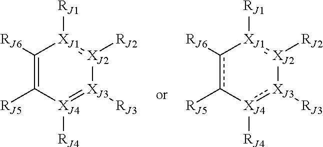

wherein:
[symbol] denotes a single or double bond;
X_{J1}-X_{J4} are selected from carbon (C) or nitrogen (N);
Where two X_{J} groups are selected from N and two X_{J} groups are selected from C such that there are two nitrogens and four carbons in the six membered ring. As a non-limiting example, if X_{J1} and X_{J3} are nitrogen, then X_{J2} and X_{J4} are carbon;
R_{J1}-R_{J6} are absent or an independent R_{J} group and an R_{J} group is independently selected from hydrogen (H), hydroxy (—OH), oxo (═O), methyl (—CH₃), carboxylate (—COO⁻), methoxy (—O—CH₃), ethoxy (—OCH₂CH₃), or acetoxy (—O(C═O)CH₃);
At least one of R_{J1}-R_{J6} is a carboxylate;
If an R group is oxo, then the adjacent [symbol] bond within the six membered ring is a single bond;
If an R_{J} group is bonded to nitrogen then that R_{J} group is absent or selected from hydrogen (H) or methyl (—CH₃).

In some embodiments an R_{J} group may be selected from hydrogen (H), hydroxy (—OH), oxo (═O), methyl (—CH₃), carboxylate (—COO⁻), methoxy (—O—CH₃), or acetoxy (—O(C═O)CH₃). In some embodiments an R_{J} group may be selected from hydrogen (H), hydroxy (—OH), oxo (═O), methyl (—CH₃), methoxy (—O—CH₃), or carboxylate (—COO⁻). In some embodiments an R_{J} group may be selected from hydrogen (H), oxo (═O), methyl (—CH₃), or carboxylate (—COO⁻).

In some embodiments up to 2 R_{J} groups (e.g. 1 or 2) may be a carboxylate. In some embodiments up to 2 R_{J} groups (e.g. 1 or 2) may be hydroxy. In some embodiments up to 2 R_{J} groups (e.g. 1 or 2) may be methoxy. In some embodiments up to 2 R_{J} groups (e.g. 1 or 2) may be ethoxy. In some embodiments up to 2 R_{J} groups (e.g. 1 or 2) may be acetoxy. In some embodiments up to 2 R_{J} groups (e.g. 1 or 2) may be oxo. In some embodiments up to 2 R_{J} groups (e.g. 1 or 2) may be methyl.

In some embodiments an R_{J} group may comprise one or more conjugate acid or conjugate base or one or more protonated or deprotonated forms.

A non-limiting example of a diazine carboxylate substance of [Formula 11-A] is pyrimidine-2-carboxylate, wherein X_{J1} and X_{J3} are nitrogen, X_{J2} and X_{J4} are carbon, R_{J1} and R_{J3} are absent, R_{J4}—R_{J6} are hydrogen, and R_{J2} is carboxylate.

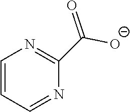

A non-limiting example of a diazine carboxylate substance of [Formula 11-A] is pyrazinecarboxylate (also known as pyrazine-2-carboxylate), wherein X_{J1} and X_{J4} are nitrogen, X_{J2} and X_{J3} are carbon, R_{J1} and R_{J4} are absent, R_{J3}, R_{J5}, and R_{J6} are hydrogen, and R_{J2} is carboxylate.

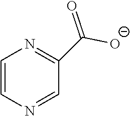

In some embodiments an RNA stabilizing substance may comprise a pyrimidine carboxylate substance. In some embodiments a diazine carboxylate substance may comprise a pyrimidine carboxylate substance. In some embodiments an RNA stabilizing substance may comprise a pyrimidine carboxylate substance that has the formula [Formula 11-B1 or 11-B2]:

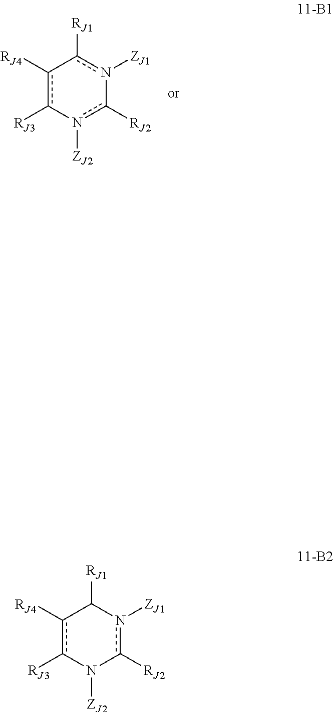

wherein:
[symbol] denotes a single or double bond;
R_{J1}-R_{J4} are independent Ry groups described in this section;
At least one of R_{J}-R_{J4} is a carboxylate;
If an R group is oxo, then the adjacent [symbol] bond within the six membered ring is a single bond;
Z_{J1} and Z_{J2} are absent or independent Z_{J} groups and a Z_{J} group is selected from hydrogen (H) or methyl (—CH₃).

A non-limiting example of a pyrimidine carboxylate substance of [Formula 11-B1] is orotate (also known as a conjugate base of orotic acid) wherein, R_{J1} and R_{J2} are oxo, R_{J3} is carboxylate, and R_{J4} is H, and Z_{J1} and Z_{J2} are H.

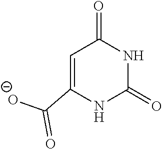 [SMILES: O=C([O-])c1cc(=O)[nH]c(=O)[nH]1]

In some embodiments other diazine carboxylate substances may include: orotate, dihydro orotate (also known as 4,5-dihydroorotate), pyrazinecarboxylate (also known as pyrazine-2-carboxylate), pyrimidine-2-carboxylate, pyrimidine-4-carboxylate, pyrimidine-5-carboxylate, pyrazine-2,3-dicarboxylate, pyrimidine-2,4-dicarboxylate, pyrimidine-2,5-dicarboxylate, or pyrimidine-4,6-dicarboxylate.

In some embodiments a pyrimidine carboxylate substance may be a pyrimidine dicarboxylate, wherein a pyrimidine dicarboxylate may include: pyrimidine-2,4-dicarboxylate, pyrimidine-2,5-dicarboxylate, or pyrimidine-4,6-dicarboxylate.

In some embodiments a pyrimidine carboxylate substance may be a pyrimidine carboxylate, wherein a pyrimidine carboxylate may include: orotate, dihydro orotate (also known as 4,5-dihydroorotate), pyrimidine-2-carboxylate, pyrimidine-4-carboxylate, or pyrimidine-5-carboxylate.

Embodiments comprising diazine carboxylate substances or pyrimidine carboxylate substances, may include one or more conjugate acid or conjugate base, tautomer, or salt thereof.

In some embodiments an RNA stabilizing substance comprising a diazine carboxylate substance or pyrimidine carboxylate substance may be used in or to produce one or more RNA stabilizing composition described herein. In some embodiments an RNA stabilizing substance comprising a diazine carboxylate substance or pyrimidine carboxylate substance may be used in or to produce one or more RNA stabilizing composition described herein, where the concentration of a diazine carboxylate substance or pyrimidine carboxylate substance may be between about 5 mM-1M, or between about 5 mM-500 mM, or between about 5 mM-300 mM, as non-limiting examples (e.g. about 5 mM, 10 mM, 20 mM, 50 mM, 100 mM, 150 mM, 200 mM, 300 mM, 400 mM, 500 mM or 1M, as non-limiting examples).

### Acyclic Carboxylate Section

In some embodiments an RNA stabilizing substance may comprise an acyclic carboxylate substance. In some embodiments an RNA stabilizing substance may comprise an acyclic carboxylate substance that has the formula [Formula 12]:

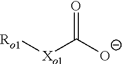

wherein:
O is oxygen;
X_{O1} is an independent X_{O} group and an X_{O} group is absent or an independent C₁₋₁₀ alkyl or alkenyl group, that is optionally substituted with up to five of hydroxy or oxo;

If X_{O1} is absent, then R_{O1} must be present and is bonded directly to the carboxylate (—COO⁻) in place of X_{O1};

R_{O1} is an independent R_{O} group and an R_{O} group is independently selected from hydrogen (—H), hydroxy (—OH), or carboxylate (—COO⁻); If X_{O1} is absent, then R_{O1} is carboxylate (—COO⁻);

If X_{O1} is a C₁ alkyl (e.g. CH₂), then R_{O1} is selected from hydroxy (—OH) or carboxylate (—COO⁻);

If X_{O1} is a C₆₋₁₀ alkyl or alkenyl group, then R_{O1} is carboxylate.

In some embodiments if X_{O1} is a C₅₋₁₀ alkyl or alkenyl group, then R_{O1} is carboxylate. In some embodiments if X_{O1} has greater than 6 carbons, then R_{O1} is carboxylate. In some embodiments if X_{O1} has greater than 5 carbons, then R_{O1} is carboxylate.

In some embodiments an X_{O} group may be a C₁₋₁₀ alkyl or alkenyl group, that is optionally substituted with up to five of hydroxy or oxo (e.g. 1, 2, 3, 4, or 5, of hydroxy or oxo). In some embodiments an X_{O} group may be a C₁₋₈ alkyl group, that is optionally substituted with one or two of hydroxy or oxo. In some embodiments an X_{O} group may be a C₁₋₆ alkyl group, that is optionally substituted with one or two of hydroxy or oxo. In some embodiments an X_{O} group may be a C₂₋₁₀ alkyl or alkenyl group, that is optionally substituted with up to five of hydroxy or oxo (e.g. 1, 2, 3, 4, or 5, of hydroxy or oxo). In some embodiments an X_{O} group may be a C₂₋₈ alkyl group, that is optionally substituted with one or two of hydroxy or oxo. In some embodiments an X_{O} group may be a C₂₋₆ alkyl group, that is optionally substituted with one or two of hydroxy or oxo. In some embodiments an X_{O} group may comprise 1 double bond, or up to 2, or up to 3 double bonds (e.g. 1, 2, or 3 double bonds).

In some embodiments an X_{O} group may comprise a carbon chain with 1-10, 1-8 1-6, or 1-4 carbons (e.g. 1, 2, 3, 4, 5, 6, 7, 8, 9 or 10 carbons). In some embodiments an X_{O} group may comprise a carbon chain with 2-10, 2-8, or 2-6 carbons (e.g. 2, 3, 4, 5, 6, 7, 8, 9, or 10 carbons). In some embodiments an X_{O} group may be saturated, monounsaturated, or polyunsaturated. In some embodiments an X_{O} group may be substituted with 1-5, or 1-4, or 1-2 hydroxy or oxo (e.g. 1, 2, 3, 4, or 5 hydroxy or oxo).

In some embodiments [Formula 12] or an R_{O} group may comprise one or more conjugate acid or conjugate base or one or more protonated or deprotonated forms.

A non-limiting example of an acyclic carboxylate substance of [Formula 12] is nonanedioate (also known as a conjugate base of azelaic acid) where X_{O1} is (CH₂) 7, and R_{O1} is carboxylate.

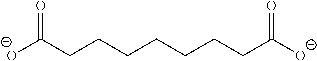 [SMILES: O=C([O-])CCCCCCCC(=O)[O-]]

A non-limiting example of an acyclic carboxylate substance of [Formula 12] is glycolate where X_{O1} is CH₂, and R_{O1} is hydroxy.

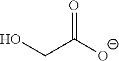

In some embodiments other acyclic carboxylate substances may include: glycolate, butanoate, propanoate, pentanoate, oxalate, 2-hydroxypropanoate, 3-hydroxypropanoate, 2-hydroxybutanoate, 3-hydroxybutanoate, 4-hydroxybutanoate, 2-hydroxypentanoate, 3-hydroxypentanoate, 4-hydroxypentanoate, 5-hydroxypentanoate, 1-hydroxyhexanoate, 2-hydroxyhexanoate, 3-hydroxyhexanoate, 4-hydroxyhexanoate, 5-hydroxyhexanoate, 6-hydroxyhexanoate, propanedioate (also known as malonate), butanedioate (also known as succinate), pentanedioate (also known as glutarate), hexanedioate (also known as adipate), heptanedioate (also known as pimelate), octanedioate (also known as suberate), or nonanedioate (also known as deprotonated azelaic acid).

Embodiments comprising acyclic carboxylate substances, may include one or more conjugate acid or conjugate base, or salt thereof.

In some embodiments an RNA stabilizing substance comprising an acyclic carboxylate substance may be used in or to produce one or more RNA stabilizing composition described herein. In some embodiments an RNA stabilizing substance comprising an acyclic carboxylate substance may be used in or to produce one or more RNA stabilizing composition described herein, where the concentration of an acyclic carboxylate substance may be between about 5 mM-2M, or between about 5 mM-1M, or between about 10 mM-500 mM, as non-limiting examples (e.g. about 10 mM, 20 mM, 50 mM, 100 mM, 150 mM, 200 mM, 300 mM, or 500 mM, as non-limiting examples).

### Ascorbic Acid Derivatives Section

In some embodiments an RNA stabilizing substance may comprise an ascorbic acid derivative substance. In some embodiments an RNA stabilizing substance may comprise an ascorbic acid derivative substance that has the formula [Formula 13-A]:

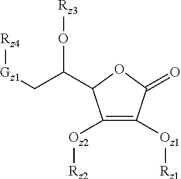

wherein
O_{Z1} and O_{Z2} are oxygen (O);
G_{Z1} is selected from oxygen (O) or NH;
R_{Z1} is absent or an independent R_{Z} group;
R_{Z2} is absent or an independent R_{Z} group;
R_{Z3} and R_{Z4} are independent R_{Z} groups;
An R_{Z} group is independently selected from hydrogen (H) or the following:

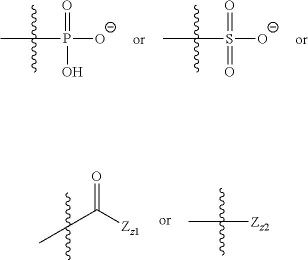

where Z_{Z1} and Z_{Z2} are independent Z_{Z} groups and a Z_{Z} group is independently selected from C₁₋₆ alkyl or alkenyl group, that is optionally substituted with one, two, three, four, or five hydroxys.

In some embodiments R_{Z1} may be further selected from the following glucopyranosyl group:

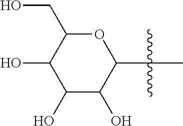 [SMILES: *C1OC(CO)C(O)C(O)C1O]

In some embodiments O_{Z1} and O_{Z2} may optionally be oxidized, wherein if O_{Z1} and O_{Z2} are oxidized then the corresponding R_{Z} groups (R_{Z1} and R_{Z2}) are both absent and the bond between the two adjacent carbon atoms bonded to O_{Z1} and O_{Z2} is a single bond as shown in the following non-limiting example formula [Formula 13-B]:

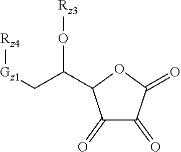

Where G_{Z1} is selected from oxygen (O) or NH;
R_{Z3} and R_{Z4} are independent R_{Z} groups as described herein.

In some embodiments one of R_{Z1} or R_{Z2} may be absent. In some embodiments both of R_{Z1} and R_{Z2} may be absent.

In some embodiments a Z_{Z} group may be a C₁₋₆ alkyl group, that is optionally substituted with one or two hydroxy. In some embodiments a Z_{Z} group may be a C₁₋₆ alkyl group, that is optionally substituted with one hydroxy. In some embodiments a Z_{Z} group may be a C₁₋₄ alkyl group, that is optionally substituted with one or two hydroxys. In some embodiments a Z_{Z} group may be a C₂₋₆ alkyl group, that is optionally substituted with one, two, three, four, or five hydroxys.

In some embodiments a Z_{Z} group may be a C₂₋₆ alkyl group, that is optionally substituted with one, or two hydroxys. In some embodiments a Z_{Z} group may be a C₂₋₄ alkyl group, that is optionally substituted with one or two hydroxys. In some embodiments a Z_{Z} group may comprise 1 double bond, or up to 2, or up to 3 double bonds (e.g. 1, 2, or 3 double bonds).

In some embodiments a Z_{Z} group may comprise a carbon chain with 1-6 or 1-4 carbons (e.g. 1, 2, 3, 4, 5, or 6 carbons). In some embodiments a Z_{Z} group may comprise a carbon chain with 2-6 or 2-4 carbons (e.g. 2, 3, 4, 5, or 6 carbons).

In some embodiments [Formula 9-A] or an R_{Z} group may comprise one or more conjugate acid or conjugate base or one or more protonated or deprotonated forms.

A non-limiting example of an ascorbic acid derivative substance of [Formula 13-A] is 2-phospho-L-ascorbic acid where G_{Z1} is O, R_{Z2}—R_{Z4} are H and R_{Z1} is (—PO₃⁻H).

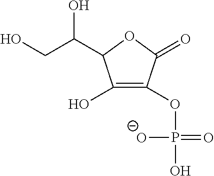 [SMILES: O=C1OC(C(O)CO)C(O)=C1OP(=O)([O-])O]

A non-limiting example of an ascorbic acid derivative substance of [Formula 13-A] is L-ascorbic acid 2,6 dibutyrate where G_{Z1} is O, R_{Z1} and R_{Z4} are both-(C═O)—Z_{Z1} and Z_{Z1} is a C₃ alkyl, and R_{Z2} and R_{Z3} are both H.

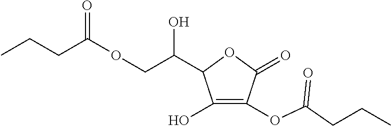 [SMILES: CCCC(=O)OCC(O)C1OC(=O)C(OC(=O)CCC)=C1O]

Other non-limiting examples of ascorbic acid derivative substances may include the following:

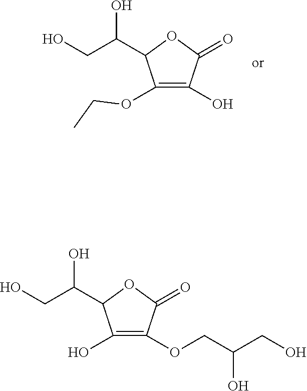 [SMILES: CCOC1=C(O)C(=O)OC1C(O)CO ; O=C1OC(C(O)CO)C(O)=C1OCC(O)CO]

Ascorbic acid derivative substances include isoascorbic acid. Ascorbic acid derivative substances include conjugate bases of ascorbic acid (e.g ascorbate or isoascorbate).

In some embodiments other ascorbic acid derivative substances may include: 3-O-methyl-ascorbic acid, 3-O-ethyl-ascorbic acid, 3-O-propyl-ascorbic acid, 3-O-butyl-ascorbic acid, 2-O-methyl-ascorbic acid, 2-O-ethyl-ascorbic acid, 2-O-propyl-ascorbic acid, 2-O-butyl-ascorbic acid, 2-O-(2,3-dihydroxypropyl) ascorbic acid, 3-O-(2,3-dihydroxypropyl) ascorbic acid, 2-O-alpha-D-glucopyranosyl-ascorbic acid, 2-phospho-ascorbic acid, 3-phospho-ascorbic acid, ascorbyl 2,6-dibutyrate, or (+)-5,6-O-isopropylidene-L-ascorbic acid.

Embodiments comprising ascorbic acid derivative substances, may include one or more conjugate acid or conjugate base, tautomer, stereoisomer, or salt thereof. Stereoisomers may include isoascorbic acid, or L-isomers or D-isomers.

In some embodiments an RNA stabilizing substance comprising an ascorbic acid derivative substance may be used in or to produce one or more RNA stabilizing composition described herein. In some embodiments an RNA stabilizing substance comprising an ascorbic acid derivative substance may be used in or to produce one or more RNA stabilizing composition described herein, where the concentration of an ascorbic acid derivative substance may be between about 5 mM-2M, or between about 10 mM-1M, or between about 50 mM-1M, as non-limiting examples (e.g. about 50 mM, 100 mM, 200 mM, 300 mM, 500 mM, 800 mM, or 1M, as non-limiting examples).

### Modified Amino Acids

In some embodiments an RNA stabilizing substance may comprise a modified amino acid substance. In some embodiments an RNA stabilizing substance may comprise a modified amino acid substance that has the formula [Formula 14-A]:

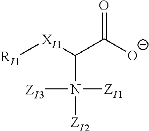

wherein:
Z_{I1} is selected from hydrogen (H), methyl (—CH₃), or acetyl (—(C═O)CH₃);
Z_{I2} is selected from hydrogen (H) or methyl (—CH₃);
Z_{I3} is absent or selected from methyl (—CH₃);
X_{I1} is an X_{I} group and an X_{I} group is independently selected from hydrogen (H) or a C₁₋₄ alkyl group, that is optionally substituted with one or two of hydroxy or oxo;
If X_{I1} is H, then R_{I1} is absent;
R_{I1} is absent or selected from hydrogen (H), hydroxy (—OH), amino (—NH₂), carboxylate (—COO⁻), amide (—(C═O)—NH₂), phenyl, p-hydroxyphenyl, methylthio (—S—CH₃), or the following:

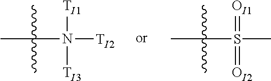

where
T_{I1} is selected from hydrogen (H), methyl (—CH₃), or acetyl (—(C═O)CH₃);
T_{I2} is selected from hydrogen (H) or methyl (—CH₃);
T_{I3} is absent or selected from methyl (—CH₃);
O_{I1} is absent or oxygen (O);
O_{I2} is absent or oxygen (O);

In some embodiments Rn may be selected from hydrogen (H), hydroxy (—OH), amino (—NH₂), carboxylate (—COO⁻), p-hydroxyphenyl, or (—S—CH₃). In some embodiments R_{I1} may be selected from amino (—NH2), carboxylate (—COO⁻), p-hydroxyphenyl, (—S—CH₃), —(O_{I1}=S=O_{I2})—CH₃, or —(N-(T_{I1-3}).

In some embodiments when Rn is hydroxy (—OH) or p-hydroxyphenyl, the hydroxy group or the hydroxy on the p-hydroxyphenyl may be substituted with phosphate (—OPO₃⁻H).

In some embodiments an X_{I} group may be a C₁₋₄ alkyl group, that is optionally substituted with one of hydroxy or oxo. In some embodiments and X_{I} group may be a C₂₋₄ alkyl group, that is optionally substituted with one of hydroxy or oxo. In some embodiments an X_{I} group may be a C₃₋₄ alkyl group, that is optionally substituted with one hydroxy.

In some embodiments an X_{I} group may comprise a carbon chain with 1-4 or 2-4 carbons (e.g. 1, 2, 3, or 4 carbons). In some embodiments an X_{I} group may comprise a carbon chain with 3-4 carbons (e.g. 3 or 4 carbons).

In some embodiments an X_{I} group may be substituted with 1-2 hydroxy groups (e.g. 1 or 2). In some embodiments an X_{I} group may be substituted with 1-2 oxo groups (e.g. 1 or 2).

A non-limiting example of a modified amino acid substance of [Formula 14-A] is methionine sulfoxide where X_{I1} is (CH₂)₂, R_{I1} is —(O=S)—CH₃ where R_{I1} is —(O_{I1}=S=O_{I2})—CH₃ and On is oxygen and O_{I2} is absent, and Z_{I1}-Z_{I2} are H and Z_{I3} is absent.

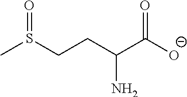

A non-limiting example of a modified amino acid substance of [Formula 14-A] is N-acetyl tyrosine (such as N-acetyl-L-tyrosine) where X_{I1} is CH₂, R_{I1} is p-hydroxyphenyl, Z_{I1} is acetyl, and Z_{I2} is H and Z_{I3} is absent.

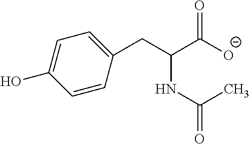 [SMILES: CC(=O)NC(Cc1ccc(O)cc1)C(=O)[O-]]

In some embodiments an RNA stabilizing substance may comprise a modified amino acid substance wherein a modified amino acid substance may include an N-acetyl amino acid, quaternary amine amino acid, tertiary amine amino acid, tertiary sulfonium amino acid, phosphorylated amino acid, methionine sulfoxide, or methionine sulfone.

In some embodiments a modified amino acid substance may comprise an N-acetyl amino acid substance. In some embodiments an RNA stabilizing substance may comprise an N-acetyl amino acid substance. In some embodiments an N-acetyl amino acid substance may comprise an N-acetyl amino acid. As a non-limiting example, an N-acetyl amino acid substance may comprise one or more of the following non-limiting example N-acetyl amino acids including: N-acetyl proline, N-acetyl tyrosine, N-acetyl methionine, N-acetyl cysteine, N-acetyl glutamate, N-acetyl aspartate, N-acetyl glutamine, N-acetyl asparagine, N-acetyl serine, N-acetyl threonine, N-acetyl valine, N-acetyl leucine, N-acetyl isoleucine, N-acetyl alanine, N-acetyl tryptophan, N-acetyl lysine (alpha or epsilon), N-acetyl histidine, N-acetyl arginine, N-acetyl phenylalanine, N-acetyl glycine, N-acetyl-S-methyl-methionine, N-acetyl methionine sulfoxide, or N-acetyl methionine sulfone.

In some embodiments one or more N-acetyl amino acid substances may include one or more stereoisomers (e.g. L-isomers or D-isomers).

Other modified amino acid substances may include S-methyl-methionine, methionine sulfoxide, methionine sulfone, N-acetyl-S-methyl-methionine, N-acetyl methionine sulfoxide, N-acetyl methionine sulfone, valine betaine, alanine betaine, epsilon-N-trimethyl lysine, epsilon-N-dimethyl lysine, O-phosphotyrosine, O-phosphoserine, and O-phosphothreonine as non-limiting examples.

Modified amino acid substances may include one or more stereoisomers (e.g. L-isomers or D-isomers).

In some embodiments a modified amino acid substance may include: N-acetyl proline, N-acetyl tyrosine, N-acetyl methionine, N-acetyl cysteine, N-acetyl glutamate, N-acetyl aspartate, N-acetyl glutamine, N-acetyl asparagine, N-acetyl serine, N-acetyl threonine, N-acetyl valine, N-acetyl leucine, N-acetyl isoleucine, N-acetyl alanine, N-acetyl tryptophan, alpha-N-acetyl lysine, epsilon-N-acetyl lysine, N-acetyl histidine, N-acetyl arginine, N-acetyl phenylalanine, or N-acetyl glycine, O-acetyl carnitine, valine betaine, alanine betaine, epsilon-N-trimethyl lysine, epsilon-N-dimethyl lysine, S-methyl-methionine, methionine sulfoxide, methionine sulfone, N-acetyl-S-methyl-methionine, N-acetyl methionine sulfoxide, N-acetyl methionine sulfone, O-phosphotyrosine, O-phosphoserine, and O-phosphothreonine.

Embodiments comprising modified amino acid substances or N-acetyl amino acid substances, may include one or more conjugate acid or conjugate base, stereoisomer, or salt thereof. Stereoisomers may include L-isomers or D-isomers.

In some embodiments an RNA stabilizing substance comprising a modified amino acid substance or an N-acetyl amino acid substance may be used in or to produce one or more RNA stabilizing composition described herein. In some embodiments an RNA stabilizing substance comprising a modified amino acid substance or an N-acetyl amino acid substance may be used in or to produce one or more RNA stabilizing composition described herein, where the concentration of a modified amino acid substance or an N-acetyl amino acid substance may be between about 10 mM-2M, or between about 20 mM-1M, or between about 50 mM-1M, as non-limiting examples (e.g. about 50 mM, 100 mM, 200 mM, 300 mM, 500 mM, 800 mM, or 1M, as non-limiting examples).

### Non-Proteinogenic Amino Acids

As used herein a non-proteinogenic amino acid refers to an amino acid that is not naturally coded in the human genetic code.

In some embodiments an RNA stabilizing substance may comprise a non-proteinogenic amino acid substance. In some embodiments an RNA stabilizing substance may comprise a non-proteinogenic amino acid substance that has the formula [Formula 14-B]:

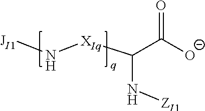

wherein:
q is an integer selected from 1-3 (e.g. 1, 2, or 3);
Z_{I1} is hydrogen (H) or acetyl (—(C═O)CH₃);
X_{Iq} is an independent X_{I} group and an X_{I} group is independently selected at each occurrence from a C₁₋₄ alkyl group, that is optionally substituted with one or two of hydroxy or oxo;
J_{I1} is selected from hydrogen (H) or amide (—(C═O)—NH₂).

In some embodiments an X_{I} group may be a C₁₋₄ alkyl group, that is optionally substituted with one of hydroxy or oxo. In some embodiments an X_{I} group may be a C₂₋₄ alkyl group, that is optionally substituted with one of hydroxy or oxo. In some embodiments an X_{I} group may be a C₃₋₄ alkyl group, that is optionally substituted with one hydroxy.

In some embodiments an X_{I} group may comprise a carbon chain with 1-4 or 2-4 carbons (e.g. 1, 2, 3, or 4 carbons). In some embodiments an X_{I} group may comprise a carbon chain with 3-4 carbons (e.g. 3 or 4 carbons).

In some embodiments an X_{I} group may be substituted with 1-2 hydroxy groups (e.g. 1 or 2). In some embodiments an X_{I} group may be substituted with 1-2 oxo groups (e.g. 1 or 2).

A non-limiting example of a non-proteinogenic amino acid substance of [Formula 14-B] is ornithine (such as L-ornithine as a non-limiting example) where q=1, X_{I1} is (CH₂)₃, J_{I1} and Z_{I1} are both H.

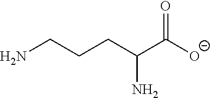

A non-limiting example of a non-proteinogenic amino acid substance of [Formula 14-B] is hypusine where q=2, X_{I1} is (CH₂)₄, X_{I2} is a C₄ alkyl substituted with one hydroxy group, and J_{I1} and Z_{I1} are both H.

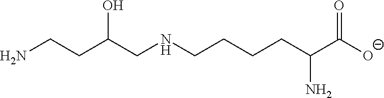 [SMILES: NCCC(O)CNCCCCC(N)C(=O)[O-]]

A non-limiting example of a non-proteinogenic amino acid substance of [Formula 14-B] is citrulline where q=1, X_{I1} is (CH₂)₃, J_{I1} is amide and Z_{I1} is H.

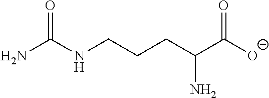 [SMILES: NC(=O)NCCCC(N)C(=O)[O-]]

Embodiments comprising non-proteinogenic amino acid substances, may include one or more conjugate acid or conjugate base, stereoisomer, or salt thereof. Stereoisomers may include L-isomers or D-isomers.

In some embodiments an RNA stabilizing substance comprising a non-proteinogenic amino acid substance may be used in or to produce one or more RNA stabilizing composition described herein. In some embodiments an RNA stabilizing substance comprising a non-proteinogenic amino acid substance may be used in or to produce one or more RNA stabilizing composition described herein, where the concentration of a non-proteinogenic amino acid substance may be between about 10 mM-2M, or between about 20 mM-1M, or between about 20 mM-500 mM, as non-limiting examples (e.g. about 20 mM, 50 mM, 100 mM, 200 mM, 300 mM, or 500 mM, as non-limiting examples).

### Sulfoxide and Sulfonyl Section

In some embodiments an RNA stabilizing substance may comprise a sulfoxide substance or a sulfonyl substance. In some embodiments an RNA stabilizing substance may comprise a sulfoxide substance or a sulfonyl substance that has the formula [Formula 15]:

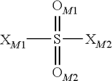

O_{M1} is oxygen (O);
O_{M2} is absent or oxygen (O);
X_{M1} and X_{M2} are independent X_{M} groups and an X_{M} group is independently selected from a C₁₋₄ alkyl group.

In some embodiments one or more X_{M} group may be a straight chain or branched. In some embodiments an X_{M} group is a methyl, ethyl, propyl, or butyl group. In some embodiments an X_{M} group may be a carbon chain with 1-4, 1-3, 2-4, or 2-3 carbons (e.g. 1, 2, 3, or 4 carbons).

A non-limiting example of a sulfonyl substance of [Formula 15] is ethyl methyl sulfone where O_{M2} is oxygen and X_{M1} is an ethyl group and X_{M2} is a methyl group.

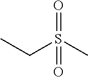

A non-limiting example of a sulfoxide substance of [Formula 15] is diethyl sulfoxide where O_{M2} is absent and X_{M1} and X_{M2} are both ethyl groups.

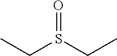

In some embodiments other sulfoxide substances or sulfonyl substances may be selected from: dimethyl sulfoxide (DMSO), diethyl sulfoxide, dipropyl sulfoxide, ethyl methyl sulfoxide, methyl propyl sulfoxide, ethyl propyl sulfoxide, dimethyl sulfone, diethyl sulfone, ethyl methyl sulfone, dipropyl sulfone, methyl propyl sulfone, and ethyl propyl sulfone.

In some embodiments an RNA stabilizing substance comprising a sulfoxide substance or a sulfonyl substance may be used in or to produce one or more RNA stabilizing composition described herein. In some embodiments an RNA stabilizing substance comprising a sulfoxide substance or a sulfonyl substance may be used in or to produce one or more RNA stabilizing composition described herein, where the weight percent concentration of a sulfoxide substance or a sulfonyl substance may be at least 5%, or at least 10%, or at least 20%, or at least 30%, or at least 40%, or at least 50%, or at least 60%, as non-limiting examples. In some embodiments an RNA stabilizing substance comprising a sulfoxide substance or a sulfonyl substance may be used in or to produce one or more RNA stabilizing composition described herein, where the concentration of a sulfoxide substance or a sulfonyl substance may be between about 50 mM-2M, or between about 50 mM-1M, or between about 100 mM-1M, as non-limiting examples (e.g. 100 mM, 200 mM, 300 mM, 500 mM, 800 mM, or 1M, as non-limiting examples).

### Polyphosphate Section

In some embodiments an RNA stabilizing substance may comprise a polyphosphate substance. In some embodiments an RNA stabilizing substance may comprise one or more polyphosphate substance that has the formula [Formula 16]:

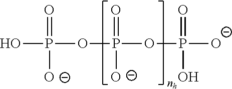

wherein:
O is oxygen and P is phosphorus;
n_{H} is an integer selected from 1-10,000.

In some embodiments nu may be selected from between about 1-10,000, 1-1,000, 1-500, 1-100, 1-50, or 1-25. In some specific embodiments nu may be selected from between about 1-500, 1-100, 1-50, 1-40, 1-30, or 1-25. In some even more specific embodiments nu may be selected from between about 1-100, 1-50, 1-40, 1-30, or 1-25.

In some embodiments nu may be greater than 5, or greater than 10, or greater than 15, or greater 20, or greater than 30, or greater than 40, or greater than 50.

Embodiments comprising polyphosphate substances, may include one or more conjugate acid or conjugate base, or salt thereof.

In some embodiments one or more polyphosphate substance may include: triphosphate or polyphosphate.

Embodiments of the present disclosure may include one or more polyphosphate substance with a molecular weight between about 1 kDa-100 kDa, or between about 1 kDa-50 kDa, or between about 5 kDa-50 kDa, or between about 1 kDa-30 kDa, or between about 5 kDa-30 kDa, as non-limiting examples (e.g. about 1 kDa, 2.5 kDa, 5 kDa, 7.5 kDa, 10 kDa, 15 kDa, 20 kDa, 25 kDa, 30 kDa, 50 kDa, or 100 kDa, as non-limiting examples).

In some embodiments an RNA stabilizing substance comprising a polyphosphate substance may be used in or to produce one or more RNA stabilizing composition described herein. In some embodiments an RNA stabilizing substance comprising a polyphosphate substance may be used in or to produce one or more RNA stabilizing composition described herein, where the concentration of a polyphosphate substance may be between about 1 mg/mL-300 mg/mL, or between about 5 mg/mL-200 mg/mL, or between about 5 mg/mL-100 mg/mL, as non-limiting examples (e.g. about 5 mg/mL, 10 mg/mL, 20 mg/mL, 30 mg/mL, 40 mg/mL, 50 mg/mL, 60 mg/mL 80 mg/mL, or 100 mg/mL, as non-limiting examples). In some embodiments an RNA stabilizing substance comprising a polyphosphate substance may be used in or to produce one or more RNA stabilizing composition described herein, where the concentration of a polyphosphate substance may be between about 5 mM-1M, or between about 10 mM-500 mM, or between about 10 mM-250 mM, as non-limiting examples (e.g. about 10 mM, 20 mM, 30 mM, 40 mM, 50 mM, 100 mM, 200 mM, or 250 mM, as non-limiting examples).

### Cyclic Phosphate Section

In some embodiments an RNA stabilizing substance may comprise a cyclic phosphate substance. In some embodiments an RNA stabilizing substance may comprise a cyclic phosphate substance that has the formula [Formula 17]:

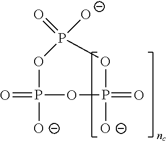

wherein:
O is oxygen and P is phosphorus;
n_{C} is an integer selected from 1-100;

In some embodiments n_{C} may be selected from between 1-50, 1-40, 1-30, 1-20, 1-10, or 1-4. In some specific embodiments n_{C} may be selected from between 1-30, 1-20, 1-10, or 1-4. In some even more specific embodiments n_{C} may be selected from between 1-10, or 1-4.

In some embodiments n_{C} may be less 50, or less than 40, or less than 30, or less 20, or less than 10. In some more specific embodiments n_{C} may be less than 30, or less 20, or less than 10. In some embodiments n_{C} may be selected from 1, 2, 3, 4, 5, 6, 7, 8, 9, or 10.

Embodiments comprising cyclic phosphate substances, may include one or more conjugate acid or conjugate base, or salt thereof.

In some embodiments one or more cyclic phosphate substance may be selected from: trimetaphosphate (TMP), tetrametaphosphate, pentametaphosphate, hexametaphosphate (HMP), heptametaphosphate, octametaphosphate, or decametaphosphate.

In some embodiments an RNA stabilizing substance comprising a cyclic phosphate substance may be used in or to produce one or more RNA stabilizing composition described herein. In some embodiments an RNA stabilizing substance comprising a cyclic phosphate substance may be used in or to produce one or more RNA stabilizing composition described herein, where the concentration of a cyclic phosphate substance may be between about 1 mg/mL-300 mg/mL, or between about 5 mg/mL-200 mg/mL, or between about 5 mg/mL-100 mg/mL, as non-limiting examples (e.g. about 5 mg/mL, 10 mg/mL, 20 mg/mL, 30 mg/mL, 40 mg/mL, 50 mg/mL, 60 mg/mL, 80 mg/mL, or 100 mg/mL, as non-limiting examples). In some embodiments an RNA stabilizing substance comprising a cyclic phosphate substance may be used in or to produce one or more RNA stabilizing composition described herein, where the concentration of a cyclic phosphate substance may be between about 5 mM-1M, or between about 10 mM-500 mM, or between about 10 mM-250 mM, as non-limiting examples (e.g. about 10 mM, 20 mM, 30 mM, 40 mM, 50 mM, 100 mM, 200 mM, or 250 mM, as non-limiting examples).

### Modified Carbohydrates Section

The inventors have surprisingly discovered that modified carbohydrate substances may stabilize RNA substances. The inventors have discovered that an RNA stabilizing substance may comprise a modified carbohydrate substance.

A modified carbohydrate substance comprises at least one or more substituents selected from quaternary amine, tertiary amine, and phosphate groups, wherein at least one or more hydroxy group on the modified carbohydrate substance is substituted with a substituent.

Modifying carbohydrates by substituting one or more hydroxy groups with different substituents is known art.

The following examples describe carbohydrate modifications that may be suitable for use:
“N. Karić, M. Vukčević, M. Ristić, A. Perić-Grujić, A. Marinković, K. Trivunac, A green approach to starch modification by solvent-free method with betaine hydrochloride, Int J Biol Macromol. 193 (2021) 1962-1971.” Referred to as modified starch, betaine modified starch, or cationic starch, wherein the synthesis procedures and methods for modifying carbohydrates is incorporated herein by reference.
“L. Passauer, F. Liebner, K. Fischer, Starch Phosphate Hydrogels. Part I: Synthesis by Mono-phosphorylation and Cross-linking of Starch, Starch-Stärke. 61 (2009) 621-627.” Referred to as modified starch, starch phosphates, monostarch monophosphates, cross-linked distarch phosphates, cross-linked monostarch monophosphates, cross-linked starch, or starch hydrogels, wherein the examples, synthesis procedures and methods for modifying carbohydrates is incorporated herein by reference.
“L. Passauer, F. Liebner, K. Fischer, Synthesis and Properties of Novel Hydrogels from Cross-linked Starch Phosphates, Macromolecular Symposia. 244 (2006) 180-193.” Referred to as modified starch, starch phosphates, monostarch monophosphates, cross-linked distarch phosphates, cross-linked monostarch monophosphates, cross-linked starch, or starch hydrogels, wherein the examples, synthesis procedures and methods for modifying carbohydrates is incorporated herein by reference.
“H. J. Prado, M. C. Matulewicz, Cationization of polysaccharides: A path to greener derivatives with many industrial applications, European Polymer Journal. 52 (2014) 53-75.” Referred to as cationized amylopectin, starch cationization, cationized chitosan, cationized hydroxyethyl cellulose, and cationic dextrans, wherein the examples, synthesis procedures and methods for modifying carbohydrates is incorporated herein by reference.

Other modified carbohydrates that may be suitable for use may include the following:
Cationic Dextran (Meito Industry Co., Ltd., Nishi Ward, Nagoya City, Aichi Prefecture, Japan Product CDC and CDC-L), (also known as dextran hydroxypropyltrimonium chloride)
Cationic dextran (CarboMer, San Diego California, SKU: 4-00658) (Also known as dextran hydroxypropyltrimonium chloride)
Polyquaternium-10 (Santa Cruz Biotechnology, Dallas, TX, Product #sc-495824) (also known as hydroxyethylcellulose ethoxylate, quaternized)
Polyquaternium-10 (The Biotek, Pasadena, CA, Cat #BT-1484482) (also known as hydroxyethylcellulose ethoxylate, quaternized)
Diethylaminoethyl-Dextran (DEAE-Dextran) (˜10 kDa) (Millipore-Sigma product #00898)
Guar Hydroxypropyltrimonium Chloride (also known as guar gum-2-hydroxy-3-(trimethylammonio) propyl ether, cationic guar, or guarcat) (Ashland Inc., Wilmington, DE, product #N-Hance 3215 cationic guar)

### Substituted Monosaccharide Section

In some embodiments an RNA stabilizing substance may comprise a substituted monosaccharide substance. In some embodiments a modified carbohydrate substance may comprise a substituted monosaccharide substance.

In some embodiments a substituted monosaccharide substance may comprise a substituted pyranose substance. In some embodiments an RNA stabilizing substance may comprise a substituted pyranose substance that has the formula [Formula 18-A]:

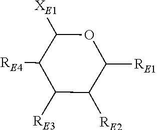

wherein:
R_{E1}-R_{E4} are independent R_{E} groups and an R_{E} group is independently selected at each occurrence from hydroxy (—OH) or the following groups:

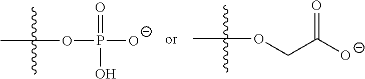 [SMILES: *OP(=O)([O-])O ; *OCC(=O)[O-]]

X_{E1} is an independent X_{E} group and an X_{E} group is independently selected at each occurrence from hydroxymethyl (—CH₂—OH), carboxylate (—COO⁻), or the following groups:

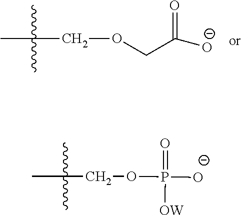

and at least one of an R_{E} group or X_{E} group is not hydroxy (—OH) or hydroxymethyl (—CH₂—OH) (e.g at least one R_{E} group is selected from —(OPO₃⁻H), —O—CH₂—(COO⁻); or at least one X_{E} group is selected from —CH₂—(OPO₃⁻H), —CH₂—O—CH₂—(COO⁻), or —(COO⁻)).

In some embodiments an R_{E} group may be selected from hydroxy (—OH), —(OPO₃⁻H), or —O—CH₂—(COO⁻). In some embodiments an R_{E} group may be selected from hydroxy (—OH) or —(OPO₃⁻H).

In some embodiments an X_{E} group may be selected from hydroxymethyl (—CH₂—OH), carboxylate (—COO⁻), —CH₂—(OPO₃⁻H), or —CH₂—O—CH₂—(COO⁻). In some embodiments an X_{E} group may be selected from hydroxymethyl (—CH₂—OH), carboxylate (—COO⁻), or —CH₂—(OPO₃⁻ H). In some embodiments an X_{E} group may be selected from hydroxymethyl (—CH₂—OH) or —CH₂—(OPO₃⁻H).

In some embodiments at least one of an R_{E} group or X_{E} group is not hydroxy or hydroxymethyl (—CH₂—OH) (e.g at least one R_{E} group is selected from —(OPO₃⁻H), —O—CH₂—(COO⁻); or at least one X_{E} group is selected from —CH₂—(OPO₃⁻H), —CH₂—O—CH₂—(COO⁻), or —(COO⁻)).

In some embodiments at least one R_{E} group is —(OPO₃⁻H), —O—CH₂—(COO⁻); or at least one X_{E} group is —CH₂—(OPO₃⁻H), —CH₂—O—CH₂—(COO⁻), or —(COO⁻)).

In some embodiments an R_{E} group or X_{E} group may comprise one or more protonated or deprotonated forms (e.g. conjugate acid or conjugate base).

In some embodiments a substituted monosaccharide substance may comprise a substituted pyranose substance. In some embodiments an RNA stabilizing substance may comprise a substituted pyranose substance that has the formula [Formula 18-B]:

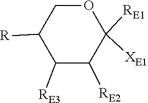

R_{E1}-R_{E4} are independent R_{E} groups described in this section;
X_{E1} is an independent X_{E} group described in this section;
and at least one of an R_{E} group or X_{E} group is not hydroxy (—OH) or hydroxymethyl (—CH₂—OH) (e.g at least one R_{E} group is selected from —(OPO₃⁻H), —O—CH₂—(COO⁻); or at least one X_{E} group is selected from —CH₂—(OPO₃⁻H), —CH₂—O—CH₂—(COO⁻), or —(COO⁻)).

In some embodiments a substituted monosaccharide substance may comprise a substituted furanose substance. In some embodiments an RNA stabilizing substance may comprise a substituted furanose substance that has the formula [Formula 18-C]:

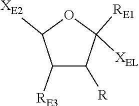

wherein:
R_{E1}-R_{E3} are independent R_{E} groups described in this section; X_{E1}—X_{E2} are independent X_{E} groups described in this section;
and at least one of an R_{E} group or X_{E} group is not hydroxy (—OH) or hydroxymethyl (—CH₂—OH) (e.g at least one R_{E} group is selected from —(OPO₃⁻H), —O—CH₂—(COO⁻); or at least one X_{E} group is selected from —CH₂—(OPO₃⁻H), —CH₂—O—CH₂—(COO⁻), or —(COO⁻)).

In some embodiments a substituted monosaccharide substance may comprise a substituted furanose substance. In some embodiments an RNA stabilizing substance may comprise a substituted furanose substance that has the formula [Formula 18-D]:

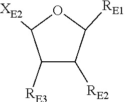

wherein:
R_{E1}-R_{E3} are independent R_{E} groups described in this section;
X_{E2} is an independent X_{E} group described in this section;
and at least one of an R_{E} group or X_{E} group is not hydroxy (—OH) or hydroxymethyl (—CH₂—OH) (e.g at least one R_{E} group is selected from —(OPO₃⁻H), —O—CH₂—(COO⁻); or at least one X_{E} group is selected from —CH₂ (OPO₃⁻H), —CH₂—O—CH₂—(COO⁻), or —(COO⁻)).

A non-limiting example of a substituted pyranose substance of [Formula 18-A] is glucose-6-phosphate (shown as beta-D-glucose-6-phosphate) wherein R_{E1}-R_{E4} are hydroxy and X_{E1} is —CH₂—(OPO₃⁻H).

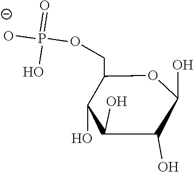

A non-limiting example of a substituted pyranose substance of [Formula 18-A] is galactose-1-phosphate (shown as alpha-D-galactose-1-phosphate) wherein R_{E1} is phosphate, R_{E2}-R_{E4} are hydroxy and X_{E1} is hydroxymethyl.

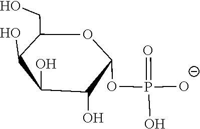

A non-limiting example of a substituted pyranose substance of [Formula 18-A] is glucuronate (also known as a conjugate base of glucuronic acid, shown here as beta-D-glucuronate) wherein R_{E1}-R_{E4} are hydroxy and X_{E1} is carboxylate.

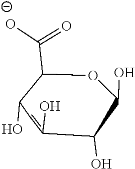

A non-limiting example of a substituted furanose substance of [Formula 18-C] is fructose-1,6-bisphosphate (also known as fructofuranose-1,6-bisphosphate, shown here as beta-D-fructose-1,6-bisphosphate) wherein R_{E1}-R_{E3} are hydroxy and X_{E1} and X_{E2} are —CH₂—(OPO₃⁻H).

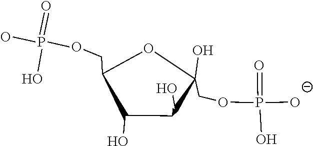

A non-limiting example of a substituted furanose substance of [Formula 18-D] is ribose-5-phosphate (shown here as beta-D-ribose-5-phosphate) wherein R_{E1}-R_{E3} are hydroxy, and X_{E2} is —CH₂—(OPO₃⁻H).

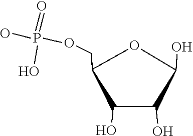

In some embodiments other substituted monosaccharide substances may include: threouronate, erythruronate, glucuronate, galacturonate, xyluronate, iduronate, taluronate, altruronate, alluronate, riburonate, arabinuronate, lyxuronate, mannuronate, or guluronate.

In some embodiments other substituted monosaccharide substances may include: combinations of glucose-1,2,3,4, or 6-phosphate (e.g. glucose-6-phosphate or glucose-1,6-phosphate), combinations of galactose-1,2,3,4, or 6-phosphate (e.g. galactose-6-phosphate or galactose-1,6-phosphate), combinations of mannose-1,2,3,4, or 6-phosphate (e.g. mannose-6-phosphate or mannose-1,6-phosphate), combinations of allose-1,2,3,4, or 6-phosphate (e.g. allose-6-phosphate or allose-1,6-phosphate), combinations of altrose-1,2,3,4, or 6-phosphate (e.g. altrose-6-phosphate or altrose-1,6-phosphate), combinations of gulose-1,2,3,4, or 6-phosphate (e.g. gulose-6-phosphate or gulose-1,6-phosphate), combinations of talose-1,2,3,4, or 6-phosphate (e.g. talose-6-phosphate or talose-1,6-phosphate), combinations of idose-1,2,3,4, or 6-phosphate (e.g. idose-6-phosphate or idose-1,6-phosphate), combinations of fructose-1,2,3,4, or 6-phosphate (e.g. fructose-6-phosphate or fructose-1,6-phosphate), combinations of psicose-1,2,3,4, or 6-phosphate (e.g. psicose-6-phosphate or psicose-1,6-phosphate), combinations of sorbose-1,2,3,4, or 6-phosphate (e.g. sorbose-6-phosphate or sorbose-1,6-phosphate), combinations of tagatose-1,2,3,4, or 6-phosphate (e.g. tagatose-6-phosphate or tagatose-1,6-phosphate), combinations of ribose-1,2,3, or 5-phosphate (e.g. ribose-5-phosphate or ribose-1,5-phosphate), combinations of arabinose-1,2,3, or 5-phosphate (e.g. arabinose-5-phosphate or arabinose-1,5-phosphate), combinations of xylose-1,2,3, or 5-phosphate (e.g. xylose-5-phosphate or xylose-1,5-phosphate), or combinations of lyxose-1,2,3, or 5-phosphate (e.g. lyxose-5-phosphate or lyxose-1,5-phosphate).

Embodiments comprising substituted monosaccharide substances, may include one or more conjugate acid or conjugate base, stereoisomers, or salt thereof. Stereoisomers may include D or L isomers or alpha or beta isomers.

In some embodiments an RNA stabilizing substance comprising a substituted monosaccharide substance may be used in or to produce one or more RNA stabilizing composition described herein. In some embodiments an RNA stabilizing substance comprising a substituted monosaccharide substance may be used in or to produce one or more RNA stabilizing composition described herein, where the concentration of a substituted monosaccharide substance may be between about 10 mM-2M, or between about 50 mM-2M, or between about 50 mM-1M, as non-limiting examples (e.g. about 10 mM, 20 mM, 50 mM, 100 mM, 150 mM, 200 mM, 300 mM, 500 mM, 1M, 1.5M, or 2M, as non-limiting examples).

### Substituted Disaccharide Section

Modified carbohydrate substances include substituted disaccharide substances.

The inventors have surprisingly discovered that substituted disaccharide substances may stabilize RNA substances. The inventors have discovered that an RNA stabilizing substance may comprise a substituted disaccharide substance.

In some embodiments an RNA stabilizing substance may comprise a substituted disaccharide substance. In some embodiments a modified carbohydrate substance may comprise a substituted disaccharide substance.

As used herein a disaccharide is dimer of two monosaccharides linked via a glycosidic bond. As a non-limiting example, a disaccharide may be sucrose wherein the monosaccharide glucose is linked to the monosaccharide fructose via a glycosidic bond.

In some embodiments a substituted disaccharide substance may comprise a disaccharide substance wherein one or more hydroxy group is substituted with one or more of the following groups:

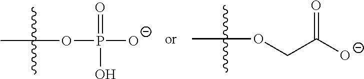 [SMILES: *OP(=O)([O-])O ; *OCC(=O)[O-]]

wherein one or more hydroxy group on one or more disaccharide substance is substituted with phosphate (—OPO₃⁻H) or carboxymethyl ether (—O—CH₂—(COO⁻)).

Non-limiting example embodiments of one or more substituted disaccharide substance may include one or more of the following, wherein two monosaccharides may be linked via a glycosidic bond and at least one hydroxy group is substituted with a phosphate group or a carboxymethyl ether group:

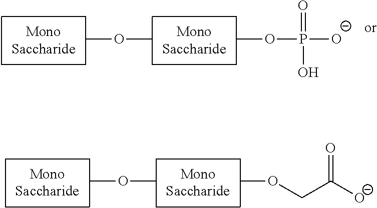

Other non-limiting example embodiments of one or more substituted disaccharide substance may include one or more of the following, wherein two monosaccharides may be linked via a glycosidic bond and at least two hydroxy groups are substituted with two different types of substituents such as one hydroxy group is substituted with a phosphate group and another hydroxy group is substituted with a carboxymethyl ether group:

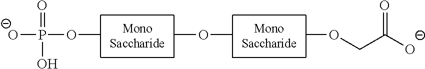

In some embodiments a substituted disaccharide substance may comprise a disaccharide substance wherein up to 2 hydroxy groups may be substituted with phosphate (—OPO₃⁻H) or carboxymethyl ether (—O—CH₂—(COO⁻)). In some embodiments a substituted disaccharide substance may comprise a disaccharide substance wherein up to 3, or up to 4 hydroxy groups, or up to 6 hydroxy groups may be substituted with phosphate (—OPO₃⁻H) or carboxymethyl ether (—O—CH₂—(COO⁻)).

In some embodiments a substituted disaccharide substance may have a degree of substitution equal to or greater than 0.5, or equal to or greater than 1, or equal to or greater than 1.5, or equal to or greater than 2. In some embodiments a substituted disaccharide substance may have a degree of substitution of 0.5-4, or 1-4, or 0.5-3, or 1-3, or 0.5-2, or 1-2, or 0.5-1. In some embodiments a substituted disaccharide substance may have a degree of substitution up to about 0.5, or up to about 1, or up to about 2, or up to about 3, or up to about 4.

In some embodiments a substituted disaccharide substance may comprise two monosaccharides linked via a glycosidic bond. In some embodiments a substituted disaccharide substance may comprise a pyranose-pyranose disaccharide linked via a glycosidic bond (such as trehalose or maltose as non-limiting examples). In some embodiments a substituted disaccharide substance may comprise a pyranose-furanose disaccharide linked via glycosidic bond (such as sucrose as a non-limiting example). In some embodiments a substituted disaccharide substance may comprise a furanose-furanose disaccharide linked via a glycosidic bond. In some embodiments a substituted disaccharide substance may comprise a pyranose-sugar alcohol disaccharide linked via a glycosidic bond (such as gluco-sorbitol or gluco-mannitol as non-limiting examples). In some embodiments a substituted disaccharide substance may comprise a furanose-sugar alcohol disaccharide linked via a glycosidic bond.

In some embodiments a substituted disaccharide substance may comprise a glycosidic bond. In some embodiments a substituted disaccharide substance may comprise a glycosidic bond wherein the glycosidic bond is beta-beta, alpha-alpha, or alpha-beta glycosidic bond. In some embodiments a substituted disaccharide substance may comprise a glycosidic bond wherein the glycosidic bond is a 1-1, 1-2, 1-3, 1-4, or 1-6 glycosidic bond. In some embodiments a substituted disaccharide substance may be comprise a glycosidic bond wherein the glycosidic bond is an alpha-1-alpha-1, alpha-1-alpha-2, alpha-1-alpha-4, alpha-1-alpha-6, beta-1-beta-1, beta-1-beta-2, beta-1-beta-4, beta-1-beta-6, alpha-1-beta-1, alpha-1-beta-2, alpha-1-beta-4, alpha-1-beta-6, beta-1-alpha-1, beta-1-alpha-2, beta-1-alpha-4, or beta-1-alpha-6 glycosidic bond.

A non-limiting example of a substituted disaccharide substance is sucrose-6-phosphate (shown as alpha-D-glucopyranosyl-(1→2)-beta-D-fructofuranoside-6-phosphate, also known as 6-O-phosphono-beta-D-fructofuranosyl-alpha-D-glucopyranoside) wherein alpha-D-glucose is linked to beta-D-fructose via an alpha-1-beta-2 glycosidic bond, the degree of substitution is 0.5, and the substituted disaccharide substance comprises a substitution of one hydroxy group at position 6 of the fructofuranosyl molecule substituted with a phosphate group.

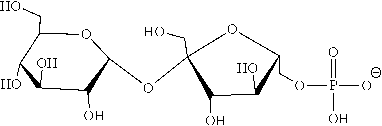

In some embodiments other substituted disaccharide substances may include: sucrose-6-phosphate, trehalose-6-phosphate, lactose-6-phosphate, dextrose-6-phosphate, maltose-6-phosphate, isomaltose-6-phosphate, cellobiose-6-phosphate, lactulose-6-phosphate, sophorose-6-phosphate, mannobiose-6-phosphate, or melibiose-6-phosphate.

Embodiments comprising substituted disaccharide substances, may include one or more conjugate acid or conjugate base, stereoisomers, or salt thereof. Stereoisomers may include D or L isomers or alpha or beta isomers.

In some embodiments an RNA stabilizing substance comprising a substituted disaccharide substance may be used in or to produce one or more RNA stabilizing composition described herein.

In some embodiments an RNA stabilizing substance comprising a substituted disaccharide substance may be used in or to produce one or more RNA stabilizing composition described herein, where the concentration of a substituted disaccharide substance may be between about 10 mM-2M, or between about 50 mM-2M, or between about 50 mM-1M, as non-limiting examples (e.g. about 10 mM, 20 mM, 50 mM, 100 mM, 150 mM, 200 mM, 300 mM, 500 mM, 1M, 1.5M, or 2M, as non-limiting examples).

### Modified Polysaccharide Substance Section

Modified carbohydrate substances include modified polysaccharide substances.

The inventors have surprisingly discovered that modified polysaccharide substances may stabilize RNA substances. The inventors have discovered that an RNA stabilizing substance may comprise a modified polysaccharide substance.

A modified polysaccharide substance comprises at least one or more substituents selected from quaternary amine, tertiary amine, and phosphate groups, wherein at least one or more hydroxy group on the modified polysaccharide substance is substituted with a substituent.

In some embodiments a modified polysaccharide substance may comprise one or more modified species of polysaccharide, including modified species of amylose, amylopectin, dextran, dextrin, or cyclodextrin as non-limiting examples, wherein the modified species of polysaccharide comprises at least one or more substituents selected from a quaternary amine, tertiary amine, or phosphate group, such that at least one or more hydroxy group on the modified species of polysaccharide is substituted with a substituent.

A modified polysaccharide substance comprises at least one or more substituents selected from quaternary amine, tertiary amine, and phosphate groups, wherein at least one or more hydroxy group on the modified polysaccharide substance is substituted with a substituent. In some embodiments a modified polysaccharide substance may comprise at least one R_{V} group substituent, wherein at least one or more hydroxy group on the modified polysaccharide substance is substituted with an R_{V} group substituent as shown in the following non-limiting example [Polysaccharide Example—1]:

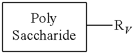

Where a [Poly Saccharide] is a modified polysaccharide substance (e.g. amylose, amylopectin, dextran, dextrin, or cyclodextrin as non-limiting examples) and an R_{V} group substituent is selected from the following groups:

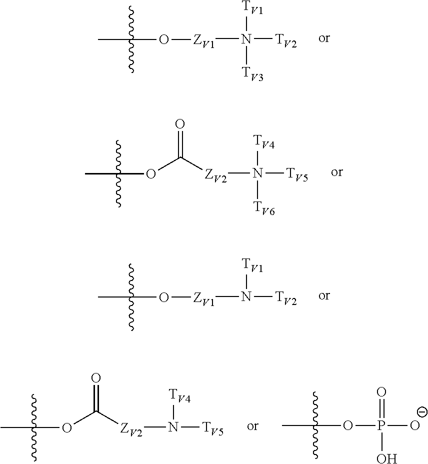

where Z_{V1} and Z_{V2} are independent Z_{V} groups and a Z_{V} group is independently selected at each occurrence from a C₁₋₆ alkyl or alkenyl group, that is optionally substituted with one or two of hydroxy or oxo or up to 2 heteroatoms;
T_{V1}-T_{V6} are independent T_{V} groups and a T_{V} group is independently selected at each occurrence from a C₁₋₄ alkyl group, that is optionally substituted with one hydroxy.

Therefore, a modified polysaccharide substance comprises at least one substituent selected from a quaternary amine, tertiary amine, or phosphate group, such that one or more hydroxy group on the modified polysaccharide is substituted with a substituent as shown in the following non-limiting example:

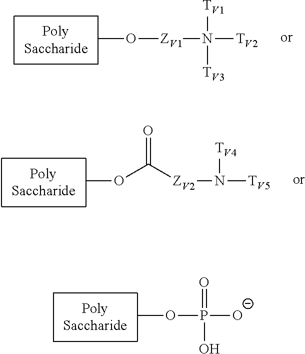

where [Poly Saccharide] is a modified polysaccharide substance;
Z_{V1}-Z_{V2} are independent Z_{V} groups as described in [Polysaccharide Example-1]; T_{V1}-Tvs are independent T_{V} groups as described in [Polysaccharide Example-1].
In some embodiments a modified polysaccharide substance may be substituted with an additional type of substituent to produce a modified polysaccharide substance with a combination of two or more types of substituents, such that at least two or more hydroxy groups on the modified polysaccharide substance are substituted with a different type of substituent.

In some embodiments a modified polysaccharide substance may be substituted with an additional type of substituent to produce a modified polysaccharide substance with a combination of two or more types of substituents, such that at least two or more hydroxy groups on the modified polysaccharide substance are substituted with a different type of substituent as shown in the following non-limiting example [Polysaccharide Example-2]:

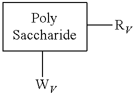

Where a [Poly Saccharide] is a modified polysaccharide substance (e.g. amylose, amylopectin, dextran, dextrin, or cyclodextrin as non-limiting examples);
An R_{V} group substituent is selected from the R_{V} groups described in [Polysaccharide Example-1];
A W_{V} group is selected from the R_{V} groups described in [Polysaccharide Example-1] and the following group:

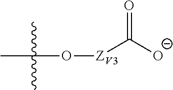

where Z_{V3} is independently selected at each occurrence from a C₁₋₆ alkyl or alkenyl group, that is optionally substituted with one or two of hydroxy or oxo or up to 2 heteroatoms;
and W_{V} is selected from a different type of substituent than R_{V}.

Therefore, in some embodiments a modified polysaccharide substance may be substituted with an additional type of substituent to produce a modified polysaccharide substance with a combination of two or more types of substituents, such that at least two or more hydroxy groups on the modified polysaccharide substance are substituted with a different type of substituent as shown in the following non-limiting examples:

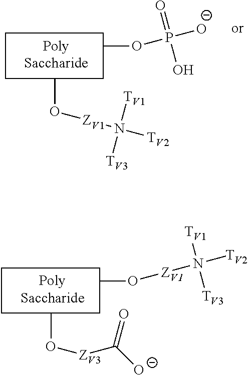

where [Poly Saccharide] is a modified polysaccharide substance;
Z_{V1} is an independent Z_{V} group as described in [Polysaccharide Example-1];
Z_{V3} is an independent Z_{V} group as described in [Polysaccharide Example-2];
T_{V1}-T_{V3} are independent T_{V} groups as described in [Polysaccharide Example-1].

In some embodiments a modified polysaccharide substance may have two or more, three or more, or four or more types of substituents (e.g. 1, 2, 3, or 4, or more types of substituents). In some embodiments a modified polysaccharide substance may be substituted with two or more of the following types of substituent groups:—O—Z_{V1}—N(T_{V1-3}), —O—(C═O)—Z_{V2}—N(T_{V4-6}), —O—Z_{V1}—N(T_{V1-2}), —O—(C═O)—Z_{V2}—N(T_{V4-5}), —(OPO₃⁻H), —O—Z_{V3}—(COO⁻). In some embodiments a modified polysaccharide substance may be substituted with a combination of two or more of the following types of substituents: a quaternary amine group, a tertiary amine group, a phosphate group, or a carboxylate group.

In some embodiments a Z_{V} group may be a C₁₋₆ alkyl group, that is optionally substituted with one or two of hydroxy or oxo. In some embodiments a Z_{V} group may be a C₁₋₄ alkyl group, that is optionally substituted with one or two of hydroxy or oxo. In some embodiments a Z_{V} group may be a C₁₋₄ alkyl group, that is optionally substituted with one of hydroxy or oxo. In some embodiments a heteroatom is selected from N or O. In some embodiments a heteroatom is O.

In some embodiments a Z_{V} group may comprise a carbon chain with 1-6, 1-4, or 1-3 carbons (e.g. 1, 2, 3, 4, 5, or 6 carbons).

In some embodiments a Z_{V} group may be saturated, monounsaturated, or polyunsaturated. In some embodiments a Z_{V} group may be substituted with between 1-2 heteroatom substituents (e.g. 1 or 2 heteroatom substituents), wherein one or two heteroatoms may be substituted for one or two carbons. In some embodiments a Z_{V} group may be substituted with 1-2 hydroxy groups or oxo groups (e.g. 1 or 2 hydroxy groups or oxo groups).

In some embodiments a T_{V} group may be a C₁₋₄ alkyl group, that is optionally substituted with one hydroxy. In some embodiments a T_{V} group may be a C₁₋₃ alkyl group, that is optionally substituted with one hydroxy. In some embodiments a T_{V} group may be a C₁₋₂ alkyl group, that is optionally substituted with one hydroxy. In some embodiments a T_{V} group may be selected from a methyl, ethyl, propyl, or butyl group. In some embodiments a T_{V} group may be an alcohol such as a methanol, ethanol, propanol, or butanol group. In some embodiments at least two T_{V} groups may be the same. In some embodiments three T_{V} groups may be the same.

In some embodiments a T_{V} group may comprise a carbon chain with 1-4, or 1-2 carbons (e.g. 1, 2, 3, or 4 carbons).

In some embodiments an R_{V} group may comprise one or more protonated or deprotonated forms (e.g. conjugate acid or conjugate base).

In some embodiments a modified polysaccharide substance may have different degrees of substitution. Degree of substitution (DS) is known in the art and is the average number of substituted hydroxy groups per polysaccharide monomer. As a non-limiting example, degree of substitution may be changed by changing the concentration of a desired substituent in relation to the polysaccharide during synthesis of a modified polysaccharide substance, such as by increasing or decreasing the concentration of the desired substituent.

In some embodiments a modified polysaccharide substance may have a DS between about 0.1-3, or between about 0.2-3, or between about 0.3-3, or between about 0.5-3, or between about 0.8-3, or between about 1-3. In some embodiments a modified polysaccharide substance may have a DS less than about 3, or less than about 2, or less than about 1.5, or less than about 1, or less than about 0.8, or less than about 0.6, or less than about 0.5, or less than about 0.4, or less than about 0.3, or less than about 0.2.

In some embodiments a modified polysaccharide substance may have a DS greater than 0.1, or greater than 0.2, or greater than 0.3, or greater than 0.4, or greater than 0.5, or greater than 0.6, or greater than 0.7, or greater than 0.8, or greater than 0.9, or greater than 1.

In some embodiments when a modified polysaccharide substance is substituted with two or more types substituents to produce a modified polysaccharide substance with a combination of two or more types of substituents, the DS of each type of substituent may be different. As a non-limiting example, a modified polysaccharide substance substituted with both a quaternary amine substituent and a phosphate substituent may have a greater DS for the quaternary amine substituent than the phosphate substituent. In some embodiments the DS of two types of substituents may be a ratio of about 1:1, 1:1.2, 1:1.5, 1:2, 1:2.5, 1:3, 1:4, 1:5, or 1:10. In some embodiments the DS of two types of substituents may be a ratio between about 1:1-10, or about 1:1-5, or about 1:1-3, or about 1:1-2.

In some embodiments a modified polysaccharide substance may comprise one or more modified species of polysaccharide, wherein the modified species of polysaccharide comprises at least one or more substituents selected from a quaternary amine, tertiary amine, or phosphate group, such that at least one or more hydroxy group on the modified species of polysaccharide is substituted with a substituent. In some embodiments a modified polysaccharide substance may comprise one or more modified species of polysaccharide wherein the polysaccharide is selected from the following species: amylose, amylopectin, dextran, dextrin, cyclodextrin (e.g. alpha, beta, gamma, or greater), cellulose, beta-glucan, mixed beta-glucan, hyaluronic acid, xanthan gum, gellan gum, guar gum, carboxymethyl cellulose, alginate, inulin, sinistrin, levan, chitosan, or chitin, or combinations thereof.

In some specific embodiments a modified polysaccharide substance may comprise one or more modified species of polysaccharide wherein the polysaccharide is selected from the following species: amylose, dextran, dextrin, cyclodextrin (e.g. alpha, beta, gamma, or greater), cellulose, beta-glucan, sinistrin, hyaluronic acid, or inulin, or combinations thereof.

In some even more specific embodiments a modified polysaccharide substance may comprise one or more modified species of polysaccharide wherein the polysaccharide is selected from the following species: amylose, dextran, dextrin, cyclodextrin (e.g. alpha, beta, gamma, or greater), or hyaluronic acid or combinations thereof.

In some embodiments of the present disclosure, one or more substituent may be selected from the following: X-(trialkylamino)alkyl, X-(trialkylamino)-1-oxoalkyl, X-(dialkylamino)alkyl, and X-(dialkylamino)-1-oxoalkyl;
wherein
X is an integer selected from 1, 2, 3, and 4;
alkyl and 1-oxoalkyl comprise exactly X carbon atoms;
alkyl is selected from methyl, ethyl, propyl, and butyl; 1-oxoalkyl is selected from carbonyl, acetyl, propionyl, and 1-oxobutyl;
trialkyl is selected from trimethyl, triethyl, tripropyl, tributyl, and ethyl-dimethyl; and dialkyl is selected from dimethyl and diethyl.

In some embodiments, one or more substituent is selected from: 2-(trimethylamino)ethyl; 2-(trimethylamino)-1-oxoethyl; and 4-(trimethylamino)-1-oxobutyl.

In some embodiments, non-limiting example tertiary and quaternary amine substituents may include:

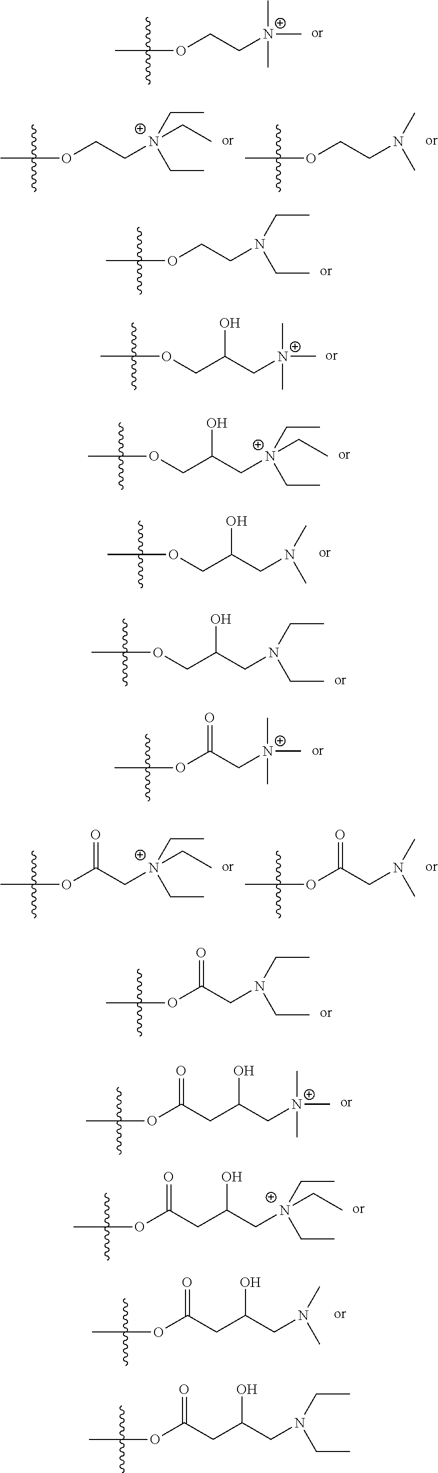

A non-limiting example of a modified polysaccharide substance is diethylaminoethyl-dextran (DEAE-dextran, such as DEAE-dextran˜10 kDa as a non-limiting example), wherein the modified polysaccharide is dextran and dextran is substituted with a substituent selected from —O—Z_{V1}—N(T_{V1}-2) where Z_{V1} is a C₂ alkyl group and T_{V1} and T_{V2} are C2 alkyl groups and the substituent is a diethylaminoethyl group. Shown here in one non-limiting example the glucose monomers within dextran are connected via an alpha-1,6-glycosidic bond with a degree of substitution of 0.5. In another non-limiting example one or more hydroxy group may be substituted at one or more position with a diethylaminoethyl group.

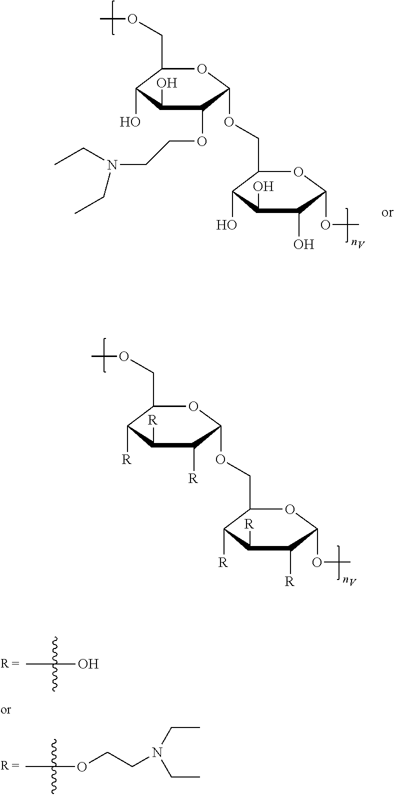

A non-limiting example of a modified polysaccharide substance is dual modified phosphate substituted diethylaminoethyl-dextran-phosphate (DEAE-dextran-phosphate), wherein the modified polysaccharide is dextran and dextran is substituted with two different types of substituents. The first substituent is selected from —O—Z_{V1}—N(T_{V1-2}) where Z_{V1} is a C2 alkyl group and T_{V1} and T_{V2} are C2 alkyl groups and the substituent is a diethylaminoethyl group; and dextran is substituted with a second substituent selected from phosphate. Shown here in one non-limiting example the glucose monomers within dextran are connected via an alpha-1,6-glycosidic bond with a degree of substitution of 1, wherein one monomer is substituted with a diethylaminoethyl group and the other monomer is substituted with a phosphate group and the ratio of phosphate to diethylamino is 1:1. In another non-limiting example one or more hydroxy group may be substituted at one or more position with a diethylaminoethyl group or a phosphate group.

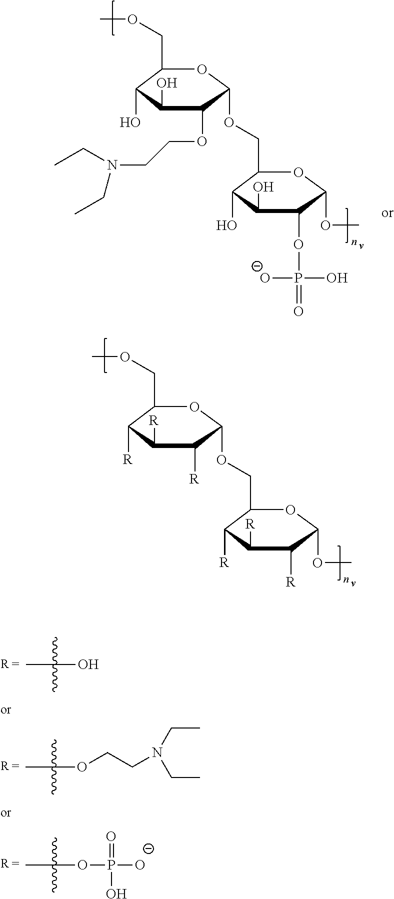

A non-limiting example of a modified polysaccharide substance is guar gum-2-hydroxy-3-(trimethylammonio) propyl-ether (also known as guar hydroxypropyltrimonium, cationic guar, or guarcat), wherein the modified polysaccharide is guar gum, and guar gum is substituted with a substituent selected from —O—Z_{V1}—N(T_{V1-3}) where Z_{V1} is a C3 alkyl group substituted with one hydroxy, and T_{V1}-T_{V3} are C1 alkyl groups and the substituent is a 2-hydroxy-3-(trimethylammonio) propyl ether group. Shown here in one non-limiting example the mannose monomers within guar gum are connected via a beta-1,4-glycosidic bond with a galactose monomer connected to a mannose monomer via an alpha-1,6-glycosidic bond with a degree of substitution of about 0.33. In another non-limiting example one or more hydroxy group may be substituted at one or more position with a 2-hydroxy-3-(trimethylammonio) propyl ether group.

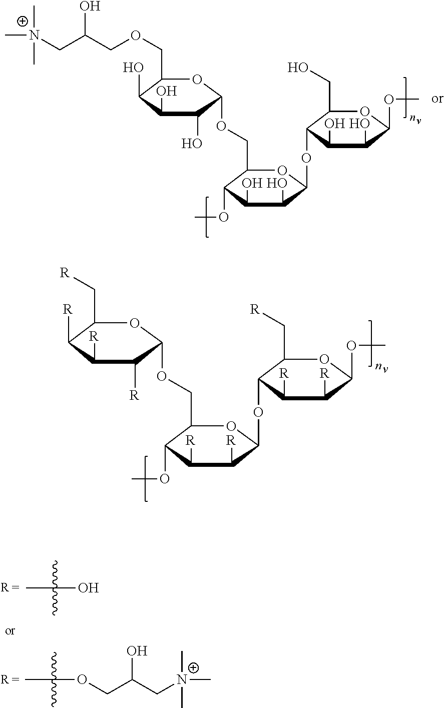

A non-limiting example of a modified polysaccharide substance is dual modified phosphate substituted guar gum-2-hydroxy-3-(trimethylammonio) propyl-ether-phosphate, wherein the modified polysaccharide is guar gum and guar gum is substituted with two different types of substituents. The first substituent is selected from —O—Z_{V1}—N(T_{V1-3}) where Z_{V1} is a C3 alkyl group substituted with one hydroxy, and T_{V1}-T_{V3} are C1 alkyl groups and the substituent is a 2-hydroxy-3-(trimethylammonio) propyl ether group; and guar gum is substituted with a second substituent selected from phosphate. Shown here in one non-limiting example the mannose monomers within guar gum are connected via a beta-1,4-glycosidic bond with a galactose monomer connected to a mannose monomer via an alpha-1,6-glycosidic bond with a degree of substitution of about 0.66, wherein one monomer is substituted with a 2-hydroxy-3-(trimethylammonio) propyl ether group and the other monomer is substituted with a phosphate group and the ratio of phosphate to 2-hydroxy-3-(trimethylammonio) propyl ether is 1:1. In another non-limiting example one or more hydroxy group may be substituted at one or more position with a 2-hydroxy-3-(trimethylammonio) propyl ether group or a phosphate group.

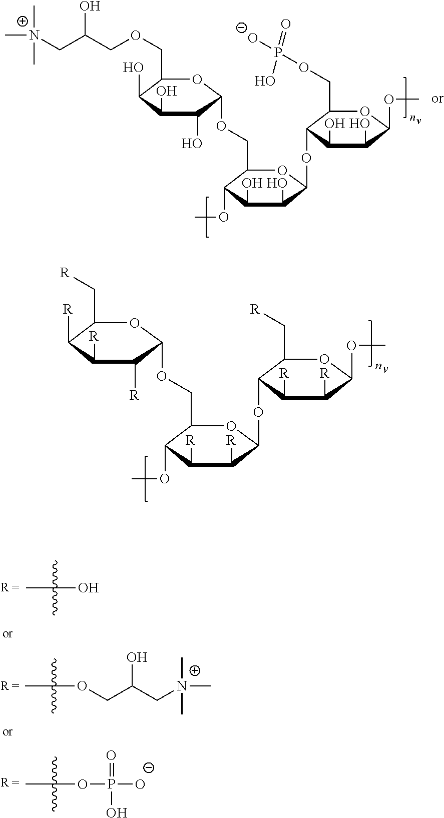

Embodiments of the present disclosure may include one or more modified polysaccharide substance with a molecular weight between about 1 kDa-1000 kDa, or between about 1 kDa-500 kDa, or between about 1 kDa-100 kDa, or between about 5 kDa-100 kDa, or between about 10 kDa-100 kDa, as non-limiting examples (e.g about 1 kDa, 2.5 kDa, 5 kDa, 7.5 kDa, 10 kDa, 15 kDa, 20 kDa, 25 kDa, 30 kDa, 50 kDa, 100 kDa, 500 kDa, or 1000 kDa, as non-limiting examples).

Embodiments comprising modified polysaccharide substances, may include one or more conjugate acid or conjugate base, stereoisomers, or salt thereof. Stereoisomers may include D or L isomers or alpha or beta isomers.

In some embodiments an RNA stabilizing substance comprising a modified polysaccharide substance may be used in or to produce one or more RNA stabilizing composition described herein. In some embodiments an RNA stabilizing substance comprising a modified polysaccharide substance may be used in or to produce one or more RNA stabilizing composition described herein, where the concentration of a modified polysaccharide substance may be between about 0.01 mg/mL-300 mg/mL, or between about 0.01 mg/mL-100 mg/mL, or between about 0.01 mg/mL-50 mg/mL, or between about 0.01 mg/mL-20 mg/mL, or between about 0.01 mg/mL-10 mg/mL, as non-limiting examples (e.g. 0.01 mg/mL, 0.02 mg/mL, 0.05 mg/mL, 0.1 mg/mL, 0.2 mg/mL, 0.5 mg/mL, 1 mg/mL, 2 mg/mL, 3 mg/mL, 4 mg/mL, 5 mg/mL, 10 mg/mL, 20 mg/mL, 50 mg/mL, 100 mg/mL, 200 mg/mL, or 300 mg/mL, as non-limiting examples). In some embodiments an RNA stabilizing substance comprising a modified polysaccharide substance may be used in or to produce one or more RNA stabilizing composition described herein, where the concentration of a modified polysaccharide substance may be less than about 300 mg/mL, or less than about 100 mg/mL, or less than about 50 mg/mL, or less than about 20 mg/mL, or less than about 10 mg/mL, or less than about 5 mg/mL, as non-limiting examples. In some embodiments an RNA stabilizing substance comprising a modified polysaccharide substance may be used in or to produce one or more RNA stabilizing composition described herein, where the concentration of a modified polysaccharide substance may be at least about 0.01 mg/mL or greater, or at least about 0.02 mg/mL or greater, or at least about 0.05 mg/mL or greater, or at least about 0.1 mg/mL or greater, or at least about 0.2 mg/mL or greater, or at least about 0.5 mg/mL or greater, or at least about 1 mg/mL or greater, or at least about 2 mg/mL or greater, or at least about 5 mg/mL or greater, as non-limiting examples.

Modified carbohydrate substances (including modified polysaccharide substances) may be modified by one or more means known in the art, which may include one or more of the following: 3-chloro-2-hydroxypropyltrimethylammonium chloride (CHPTAC), 2,3-epoxypropyltrimethylammonium chloride (glycidyltrimethylammonium chloride, GTMAC), 2-chlorotriethylamine, trimethyl glycine hydrochloride (also known as betaine hydrochloride), phosphate, polyphosphate, trimetaphosphate, carboxylic acids, dicarboxylic acids, tricarboxylic acids, or combinations thereof as non-limiting examples.

A modified carbohydrate may be a sugar alcohol, inositol, monosaccharide, or disaccharide, wherein a modified carbohydrate substance comprises at least one or more substituents selected from quaternary amine, tertiary amine, and phosphate groups, wherein at least one or more hydroxy group on the modified carbohydrate substance is substituted with a substituent.

One of ordinary skill in the art would appreciate that carbohydrates have different stereoisomers. For clarity the following descriptions may or may be not presented as different stereoisomers, however embodiments of the present disclosure may include these different stereoisomers (such as D or L isomers or alpha or beta isomers, as non-limiting examples).

One of ordinary skill in the art would appreciate that the methods of synthesis and procedures for modifying polysaccharides with different substituents provided herein may be employed in different ways and that the different features of the different types of modified polysaccharides such as the type of polysaccharide being modified, degree of substitution, or ratio of substituents may be achieved by using standard empirical approaches common in the art, non-limiting examples of such approaches may include adjusting reaction conditions (e.g. time and temperature) and concentrations of reactants among other approaches.

### Modified Sugar Alcohol Section

Modified carbohydrate substances include modified sugar alcohol substances.

The inventors have surprisingly discovered that modified sugar alcohol substances may stabilize RNA substances. The inventors have discovered that an RNA stabilizing substance may comprise a modified sugar alcohol substance.

In some embodiments an RNA stabilizing substance may comprise a modified sugar alcohol substance.

A modified sugar alcohol substance comprises at least one or more substituents selected from quaternary amine, tertiary amine, and phosphate groups, wherein at least one or more hydroxy group on the modified sugar alcohol substance is substituted with a substituent.

A modified carbohydrate substance may be a modified sugar alcohol substance with the following formula [Formula 19-A]:

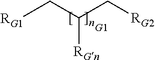

wherein
n_{G1} is an integer selected between 1-6;
R_{G1}, R_{G2} and R_{G′n} are independent R_{G} groups and an R_{G} group is independently selected at each occurrence from hydroxy (—OH) or the following groups:

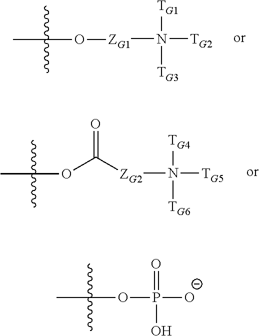

where Z_{G1}-Z_{G2} are independent Z_{G} groups and a Z_{G} group is independently selected at each occurrence from a C₁₋₆ alkyl or alkenyl group, that is optionally substituted with one or two of hydroxy or oxo;
T_{G1}, T_{G2}, T_{G4}, and T_{G5} are independent T_{G} groups and a T_{G} group is independently selected at each occurrence from a C₁₋₄ alkyl group, that is optionally substituted with one hydroxy;
T_{G3} and T_{G6} are absent or independent T_{G} groups and a T_{G} group is independently selected at each occurrence from a C₁₋₄ alkyl group, that is optionally substituted with one hydroxy;
and at least one R_{G} group is —O—Z_{G1}—N(T_{G1-3}), —O—(C═O)—Z_{G2}—N(T_{G4-6}), or —(OPO₃⁻H). In some embodiments T_{G3} or T_{G6} may be absent.

In some embodiments an R_{G} group may be selected from hydroxy (—OH), —O—Z_{G1}—N(T_{G1}-3), —O—(C═O)—Z_{G2}—N(T_{G4}-6), or —(OPO₃⁻H).

In some embodiments n_{G1} may be selected from 1-6, 1-4, 1-3, or 1-2 (e.g. 1, 2, 3, 4, 5, or 6).

In some embodiments a Z_{G} group may be a C₁₋₆ alkyl group, that is optionally substituted with one or two of hydroxy or oxo. In some embodiments a Z_{G} group may be a C₁₋₄ alkyl group, that is optionally substituted with one or two of hydroxy or oxo. In some embodiments a Z_{G} group may be a C₁₋₃ alkyl group, that is optionally substituted with one or two of hydroxy or oxo.

In some embodiments a Z_{G} group may comprise a carbon chain with 1-6, 1-4, or 1-3 carbons (e.g. 1, 2, 3, 4, 5, or 6 carbons).

In some embodiments a Z_{G} group may be saturated, monounsaturated, or polyunsaturated. In some embodiments a Z_{G} group may be substituted with 1-2 hydroxy groups or oxo groups (e.g. 1 or 2 hydroxy groups or oxo groups).

In some embodiments a T_{G} group may be a C₁₋₄ alkyl group, that is optionally substituted with one hydroxy. In some embodiments a T_{G} group may be a C₁₋₃ alkyl group, that is optionally substituted with one hydroxy. In some embodiments a T_{G} group may be a C₁₋₂ alkyl group, that is optionally substituted with one hydroxy. In some embodiments a T_{G} group may be selected from a methyl, ethyl, propyl, or butyl group. In some embodiments a T_{G} group may be an alcohol such as a methanol, ethanol, propanol, or butanol group. In some embodiments at least two T_{G} groups may be the same. In some embodiments three T_{G} groups may be the same.

In some embodiments a T_{G} group may comprise a carbon chain with 1-4, or 1-2 carbons (e.g. 1, 2, 3, or 4 carbons).

In some embodiments an R_{G} group may comprise one or more protonated or deprotonated forms (e.g. conjugate acid or conjugate base).

A non-limiting example of a modified sugar alcohol substance of [Formula 19-A] is sorbitol 6-phosphate, where n_{G1}=4, R_{G1} and R_{G′1}—R_{G′4} are OH, and R_{G2} is —(OPO₃⁻H).

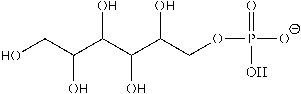 [SMILES: O=P([O-])(O)OCC(O)C(O)C(O)C(O)CO]

A modified sugar alcohol substance may include, but is not limited to, one or more of the following sugar alcohols wherein at least one hydroxy group is substituted with a phosphate, a quaternary amine, or a tertiary amine: glycerol, erythritol, threitol, arabitol, xylitol, ribitol, mannitol, sorbitol, galactitol, fucitol, iditol, or volemitol.

A modified sugar alcohol substance may include one or more of the following wherein at least one hydroxy group is substituted with a phosphate group: combinations of glycerol-1, 2, or 3-phosphate (e.g glycerol-1,3-bisphosphate or glycerol-3-phosphate), combinations of erythritol-1, 2, 3, or 4-phosphate (e.g. erythritol-4-phosphate or erythritol-1,4-bisphosphate), combinations of threitol-1, 2, 3, or 4-phosphate (e.g. threitol-4-phosphate or threitol-1,4-bisphosphate, combinations of arabitol-1, 2, 3, 4, or 5-phosphate (e.g. arabitol-5-phosphate or arabitol-3,5-bisphosphate) combinations of xylitol-1, 2, 3, 4, or 5-phosphate (e.g. xylitol-5-phosphate or xylitol-1,5-phosphate), combinations of ribitol-1, 2, 3, 4, or 5-phosphate (e.g. ribitol-5-phosphate or ribitol-2,5-bisphosphate), combinations of mannitol-1, 2, 3, 4, 5, or 6-phosphate (e.g. mannitol-6-phosphate or mannitol-1,6-bisphosphate), combinations of sorbitol-1, 2, 3, 4, 5, or 6-phosphate (e.g. sorbitol-6-phosphate or sorbitol-1,6-bisphosphate), combinations of galactitol-1, 2, 3, 4, 5, or 6-phosphate (e.g. galactitol-6-phosphate or galactitol-1,3,6-terphosphate), combinations of fucitol-2, 3, 4, 5, or 6-phosphate (e.g. fucitol-6-phosphate or fucitol-2,6-bisphosphate), combinations of iditol-1, 2, 3, 4, 5, or 6-phosphate (e.g. iditol-6-phosphate or iditol-1,6-bisphosphate), or combinations of volemitol-1, 2, 3, 4, 5, 6, or 7-phosphate (e.g. volemitol-7-phosphate or volemitol-1,7-bisphosphate).

Embodiments comprising modified sugar alcohol substances, may include one or more conjugate acid or conjugate base, stereoisomers, or salt thereof. Stereoisomers may include D isomers or L isomers.

In some embodiments an RNA stabilizing substance comprising a modified sugar alcohol substance may be used in or to produce one or more RNA stabilizing composition described herein. In some embodiments an RNA stabilizing substance comprising a modified sugar alcohol substance may be used in or to produce one or more RNA stabilizing composition described herein, where the concentration of a modified sugar alcohol substance may be between about 10 mM-3M, or between about 20 mM-2M, or between about 20 mM-1M, as non-limiting examples (e.g. 10 mM, 20 mM, 50 mM, 200 mM, 400 mM, 600 mM, 800 mM, 1M, 2M or 3M, as non-limiting examples).

### Modified Inositol Section

Modified carbohydrate substances include modified inositol substances.

The inventors have surprisingly discovered that modified inositol substances may stabilize RNA substances. The inventors have discovered that an RNA stabilizing substance may comprise a modified inositol substance.

In some embodiments an RNA stabilizing substance may comprise a modified inositol substance.

A modified inositol substance comprises at least one or more substituents selected from quaternary amine, tertiary amine, and phosphate groups, wherein at least one or more hydroxy group on the modified inositol substance is substituted with a substituent.

In some embodiments a modified carbohydrate may be a modified inositol with the following formula [Formula 19-B]:

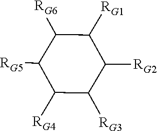

wherein:
O is oxygen;
R_{G1}—R_{G6} are independently selected R_{G} groups as described in [Formula 14-A];
and at least one R_{G} group is —O—Z_{G1}—N(T_{G1-3}), —O—(C═O)—Z_{G2}—N(T_{G4-6}), or —(OPO₃⁻H).

A non-limiting example of a modified inositol substance of [Formula 19-B] is phytate (also known as a conjugate base of phytic acid), where R_{G1}—R_{G6} are —(OPO₃⁻H).

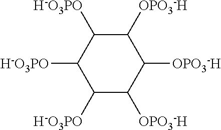

A modified inositol substance includes myo-inositol and may include one or more additional stereoisomers such as: scyllo, muco, neo, allo, epi, cis, D-chiro, or L-chiro.

In some embodiments one or more modified inositol substance may include: myo-inositol monophosphate, myo-inositol bisphosphate, myo-inositol triphosphate, myo-inositol tetraphosphate, myo-inositol pentaphosphate, or phytate (also known as a conjugate base of phytic acid). In some embodiments modified inositol substance may have 1, 2, 3, 4, 5, or 6 phosphate substituents, wherein the phosphate substituents may be one or more combinations at the 1, 2, 3, 4, 5, or 6 positions, such as myo-inositol 1,2,3,6 tetraphosphate or myo-inositol 1,3,4 triphosphate as non-limiting examples.

Embodiments comprising modified inositol substances, may include one or more conjugate acid or conjugate base, stereoisomers, or salt thereof. Stereoisomers may include myo, scyllo, muco, neo, allo, epi, cis, D-chiro, or L-chiro.

In some embodiments an RNA stabilizing substance comprising a modified inositol substance may be used in or to produce one or more RNA stabilizing composition described herein. In some embodiments an RNA stabilizing substance comprising a modified inositol substance may be used in or to produce one or more RNA stabilizing composition described herein, where the concentration of a modified inositol substance may be between about 5 mM-2M, or between about 10 mM-1M, or between about 10 mM-500 mM, as non-limiting examples (e.g. 5 mM, 10 mM, 20 mM, 50 mM, 100 mM, 200 mM, 400 mM, or 500 mM, as non-limiting examples).

### Chemically Crosslinked Carbohydrate Section

In some embodiments a modified carbohydrate substance may be crosslinked using one or more crosslinkers, where a chemical crosslinker links two or more carbohydrate units together. Chemical crosslinking of carbohydrates is known art, where one or more carbohydrate is crosslinked by the substitution of a hydroxy group on two different carbohydrate monomers by the same substituent resulting in a crosslink across the substituent and the two monomers. The following examples describe crosslinking of carbohydrates that may be suitable for use:

“L. Passauer, F. Liebner, K. Fischer, Starch Phosphate Hydrogels. Part I: Synthesis by Mono-phosphorylation and Cross-linking of Starch, Starch-Stärke. 61 (2009) 621-627.” Referred to as cross-linked distarch phosphates, cross-linked monostarch monophosphates, cross-linked starch, or starch hydrogels, wherein the synthesis procedures and methods for crosslinking carbohydrates is incorporated herein by reference.

“L. Passauer, F. Liebner, K. Fischer, Synthesis and Properties of Novel Hydrogels from Cross-linked Starch Phosphates, Macromolecular Symposia. 244 (2006) 180-193.” Referred to as cross-linked distarch phosphates, cross-linked monostarch monophosphates, cross-linked starch, or starch hydrogels, wherein the synthesis procedures and methods for crosslinking carbohydrates is incorporated herein by reference.

Non-limiting example crosslinkers that may be used to crosslink one or more modified carbohydrate substance include phosphates, cyclic phosphates, polyphosphates, aldehydes (e.g. glutaraldehyde or formaldehyde), dicarboxylic acids, tricarboxylic acids, or epoxides (e.g. epichlorohydrin).

In some embodiments a modified carbohydrate substance may be crosslinked with one or more carboxylic acids, including but not limited to one or more dicarboxylic acids, tricarboxylic acids, or higher order carboxylic acid comprising 1-5 carboxylic acid groups or greater (e.g. 1, 2, 3, 4, or 5, or more carboxylic acid groups), 1-10 carboxylic acid groups or greater, 1-20 carboxylic acid groups or greater, or 1-50 carboxylic acid groups or greater. Non-limiting example carboxylic acids that may be used to crosslink one or more modified carbohydrate substance include: maleic acid, citric acid, succinic acid, glutaric acid, pimelic acid, malonic acid, azelaic acid, tartaric acid, adipic acid, polyacrylic acid, or polymethacrylic acid, or combinations thereof.

In some embodiments a modified carbohydrate substance may be crosslinked with one or more phosphates, including but not limited to one or more diphosphates, triphosphates, polyphosphate, or cyclic phosphate comprising 1-5 phosphate groups or greater (e.g. 1, 2, 3, 4, or 5 phosphate groups or more), 1-10 phosphate groups or greater, 1-20 phosphate groups or greater, or 1-50 phosphate groups or greater. Non-limiting example phosphates that may be used to crosslink one or more modified carbohydrate substance include: phosphate, orthophosphate, diphosphate, triphosphate, polyphosphate, pyrophosphate, trimetaphosphate, tetrametaphosphate, or hexametaphosphate, or combinations thereof.

In some embodiments a modified carbohydrate substance may be crosslinked with one or more additional types of crosslinkers, such as an epoxy, aldehyde, azide, acrylamide, carboxylic acid, phosphate, phosphorus oxychloride, or combinations thereof.

In some embodiments a modified carbohydrate substance may be crosslinked with one or more additional crosslinkers, including but not limited to one or more of the following: glutaraldehyde, phosphoryl chloride, phosphorus oxychloride, epichlorohydrin, formaldehyde, boric acid, N,N-methylenebis(acrylamide), diethylene glycol diglycidyl ether, or poly(ethylene glycol) diglycidyl ether, or combinations thereof.

### Aldaric Acid Section

Aldaric acid substances as described herein may include one or more conjugate acid or conjugate base. The following descriptions of aldaric acid substances depict a conjugate base of an aldaric acid substance.

In some embodiments an RNA stabilizing substance may comprise an aldaric acid substance. In some embodiments an RNA stabilizing substance may comprise an aldaric acid substance with the following formula [Formula 20-A]:

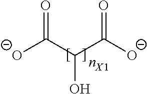

wherein:
O is oxygen;
n_{X1} is an integer selected between 1-6.

A non-limiting example of an aldaric acid of [Formula 20-A] is tartrate, where n_{X1=2}.

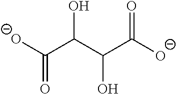

In some embodiments one or more aldaric acid substance may include: glucarate, galactarate, xylarate, idarate, talarate, altrarate, allarate, tartrate, ribarate, arabinarate, lyxarate, mannarate, gularate, or tartronate.

Embodiments comprising aldaric acid substances, may include one or more conjugate acid or conjugate base, stereoisomers, or salt thereof. Stereoisomers may include D isomers or L isomers.

In some embodiments an RNA stabilizing substance comprising an aldaric acid substance may be used in or to produce one or more RNA stabilizing composition described herein. In some embodiments an RNA stabilizing substance comprising an aldaric acid substance may be used in or to produce one or more RNA stabilizing composition described herein, where the concentration of an aldaric acid substance may be between about 5 mM-2M, or between about 20 mM-1M, or between about 50 mM-500 mM, as non-limiting examples (e.g. 5 mM, 10 mM, 20 mM, 50 mM, 100 mM, 200 mM, 300 mM, 500 mM, 600 mM, 800 mM, 1M, or 2M, as non-limiting examples).

### Aldonic Acid Section

Aldonic acid substances as described herein may include one or more conjugate acid or conjugate base. The following descriptions of aldonic acid substances depict a conjugate base of an aldonic acid substance.

In some embodiments an RNA stabilizing substance may comprise an aldonic acid substance. In some embodiments an RNA stabilizing substance may comprise an aldonic acid substance with the following formula [Formula 20-B]:

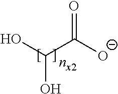

wherein:
O is oxygen;
n_{X2} is an integer selected between 1-6.

A non-limiting example of an aldonic acid of [Formula 20-B] is gluconic acid, where n_{X2}=4.

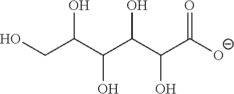 [SMILES: O=C([O-])C(O)C(O)C(O)C(O)CO]

In some embodiments one or more aldonic acid substance may include: glycerate, threonate, erythronate, gluconate, galactonate, xylonate, idonate, talonate, altronate, allonate, ribonate, arabinonate, lyxonate, mannonate, or gulonate.

Embodiments comprising aldonic acid substances, may include one or more conjugate acid or conjugate base, stereoisomers, or salt thereof. Stereoisomers may include D isomers or L isomers.

In some embodiments an RNA stabilizing substance comprising an aldonic acid substance may be used in or to produce one or more RNA stabilizing composition described herein. In some embodiments an RNA stabilizing substance comprising an aldonic acid substance may be used in or to produce one or more RNA stabilizing composition described herein, where the concentration of an aldonic acid substance may be between about 5 mM-2M, or between about 20 mM-1M, or between about 50 mM-500 mM, as non-limiting examples (e.g., 5 mM, 10 mM, 20 mM, 50 mM, 100 mM, 200 mM, 300 mM, 500 mM, 600 mM, 800 mM, 1M, or 2M, as non-limiting examples).

### Stabilizing Polymer Section

In some embodiments an RNA stabilizing substance may comprise a stabilizing polymer substance. In some embodiments an RNA stabilizing substance may comprise a stabilizing polymer substance with one of the following formulas [Formula 21-A] or [Formula 21-B]:

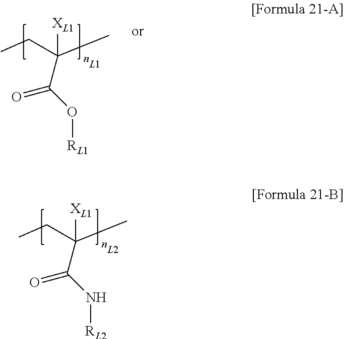

wherein
n_{L1} and n_{L2} are independent integers selected from 2-10,000;
X_{L1} is selected from hydrogen (H) or methyl (—CH₃);
R_{L1} and R_{L2} are independent R_{L} groups and an R_{L} group is independently selected at each occurrence from the following groups:

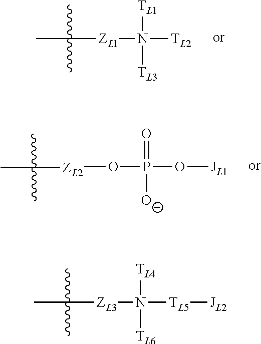

where Z_{L1}-Z_{L3} are independent Z_{L} groups and a Z_{L} group is independently selected at each occurrence from a C₁₋₈ alkyl or alkenyl group, that is optionally substituted with one or two of hydroxy or oxo or up to 2 heteroatoms;
T_{L1}-T_{L6} are independent T_{L} groups and a T_{L} group is independently selected at each occurrence from a C₁₋₄ alkyl group, that is optionally substituted with one hydroxy;

In some embodiments T_{L3} or T_{L6} may be absent;
J_{L1} and J_{L2} are independent J_{L} groups and a J_{L} group is independently selected at each occurrence from the following groups:

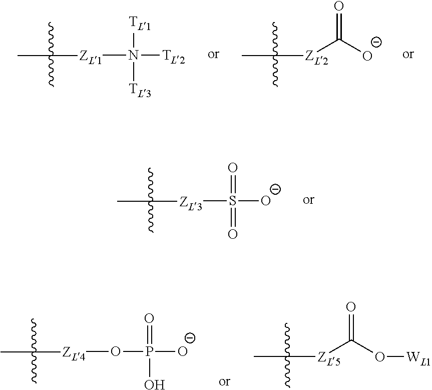

where Z_{L′1}-Z_{L′5} are independent Z_{L} groups and a Z_{L} group is independently selected at each occurrence from a C₁₋₈ alkyl or alkenyl group, that is optionally substituted with one or two of hydroxy or oxo or up to 2 heteroatoms; T_{L′1}-T_{L′3} are independent T_{L} groups and a T_{L} group is independently selected at each occurrence from a C₁₋₄ alkyl group, that is optionally substituted with one hydroxy;

In some embodiments T_{L′3} may be absent;
W_{L1} is an independent WL group and a WL group is selected from the following group:

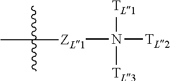

where Z_{L″1} is an independent Z_{L} group and a Z_{L} group is independently selected at each occurrence from a C₁₋₈ alkyl or alkenyl group, that is optionally substituted with one or two of hydroxy or oxo or up to 2 heteroatoms;
T_{L″1}-T_{L″3} are independent T_{L} groups and a T_{L} group is independently selected at each occurrence from a C₁₋₄ alkyl group, that is optionally substituted with one hydroxy; In some embodiments T_{L″3} may be absent.

In some embodiments an R_{L} group may be selected from —Z_{L1}—N(T_{L1-3}), —Z_{L2}—(OPO₃⁻)-J_{L1}, or —Z_{L3}—N(T_{L4-6})-J_{L2}. In some embodiments an R_{L} group may be selected from —Z_{L1}—N(T_{L1-3}), or —Z_{L3}—N(T_{L4-6})-J_{L2}.

In some embodiments a J_{L} group may be selected from —Z_{L′1}—N(T_{L′1-3}), —Z_{L′2}—(COO⁻), —Z_{L′3}—(SO₃⁻), —Z_{L′4}—(OPO₃⁻H), or —Z_{L′5}—(C—O)—O—W_{L1}. In some embodiments a J_{L} group may be selected from —Z_{L′1}—N(T_{L′1-3}), —Z_{L′2}—(COO⁻), —Z_{L′3}—(SO₃⁻), or —Z_{L′5}—(C—O)—O—W_{L1}. In some embodiments a J_{L} group may be selected from —Z_{L′2}—(COO⁻), —Z_{L′3}—(SO₃⁻), or —Z_{L′5}—(C—O)—O—W_{L1}. In some embodiments a J_{L} group may be selected from —Z_{L′2}—(COO) or —Z_{L′3}—(SO₃⁻).

In some embodiments n_{L1} or n_{L2} may be selected from between about 5-1000, 5-500, 5-250, 5-100, or 5-50. In some specific embodiments n_{L1} or n_{L2} may be selected from between about 10-500, 10-250, 10-100, or 10-50. In some even more specific embodiments n_{L1} or n_{L2} may be selected from between about 10-250, 10-100, or 10-50.

In some embodiments a Z_{L} group may be a C₁₋₆ alkyl group, that is optionally substituted with one or two of hydroxy or oxo or up to 2 heteroatoms. In some embodiments a Z_{L} group may be a C₁₋₄ alkyl group, that is optionally substituted with one hydroxy or 1 heteroatom. In some embodiments a heteroatom is selected from N or O. In some embodiments a heteroatom is O.

In some embodiments a T_{L} group may be a C₁₋₄ alkyl group, that is optionally substituted with one hydroxy. In some embodiments a T group may be a C₁₋₃ alkyl group, that is optionally substituted with one hydroxy. In some embodiments a T_{L} group may be a C₁₋₂ alkyl group, that is optionally substituted with one hydroxy. In some embodiments a T_{L} group may be selected from a methyl, ethyl, propyl, or butyl group. In some embodiments a T_{L} group may be an alcohol such as a methanol, ethanol, propanol, or butanol group. In some embodiments at least two T_{L} groups may be the same. In some embodiments three T_{L} groups may be the same.

In some embodiments a Z_{L} group may comprise a carbon chain with 1-8, 1-6, 1-4, or 1-3 carbons (e.g. 1, 2, 3, 4, 5, 6, 7, or 8 carbons). In some specific embodiments a Z_{L} group may comprise a carbon chain with 1-6, 1-4, or 1-3 carbons (e.g. 1, 2, 3, 4, 5, or 6 carbons). In some embodiments a Z_{L} group may be saturated, monounsaturated, or polyunsaturated. In some embodiments a Z_{L} group may be substituted with 1-2 heteroatoms (e.g. 1 or 2 heteroatoms), wherein one or more heteroatoms may be substituted for one or more carbons.

In some embodiments a T_{L} group may comprise a carbon chain with 1-4, or 1-2 carbons (e.g. 1, 2, 3, or 4 carbons).

In some embodiments an R_{L} or J_{L} group may comprise one or more protonated or deprotonated forms (e.g. a conjugate acid or conjugate base).

In some embodiments a stabilizing polymer substance may include one or more of the following polymers:

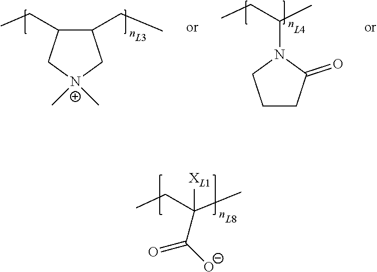

Wherein
X_{L1} is selected from H or CH₃;
n_{L3}, n_{L4}, and n_{L5} are independent integers selected from 2-10,000

In some embodiments n_{L3}, n_{L4}, and n_{L5} may be selected from between about 5-1000, 5-500, 5-250, 5-100, or 5-50. In some specific embodiments n_{L3}, n_{L4}, and n_{L5} may be selected from between about 10-500, 10-250, 10-100, or 10-50. In some even more specific embodiments n_{L3}, n_{L4}, and n_{L5} may be selected from between about 10-250, 10-100, or 10-50.

In some embodiments stabilizing polymer substance may include: poly(2-(trimethylamino)ethyl methacrylate) (PTMAEMA), hexadimethrine, poly(diallyldimethylammonium) (PDADMAC), poly(2-methacryloyloxyethyl phosphorylcholine) (PMPC), poly(vinylpyrrolidone) (PVP), poly(acrylic acid) (PAA), poly(methacrylic acid), poly(2-(diethylamino)ethyl methacrylate) (PDEAEMA), poly((carboxybetaine methacrylate)ethyl ester)) (PCBMA-ethyl ester), poly(2-(N-3-sulfopropyl-N,N-dimethyl ammonium)ethyl methacrylate) (PSBMA) (also known as poly(sulfobetaine methacrylate), poly(carboxybetaine methacrylate), poly(ethylene glycol)-block-poly(sulfobetaine methacrylate) (PEG-PSBMA), poly(ethylene glycol)-block-poly(2-methacryloyloxyethyl phosphorylcholine) (PEG-PMPC), or poly [(2-ethyldimethylammonioethyl methacrylate ethyl sulfate)-co-(1-vinylpyrrolidone)] (Polyquat 11) (also known as polyquaternium 11).

Non-limiting examples of stabilizing polymers of the present disclosure that may be suitable for use, may include those described as poly(carboxybetaine) s, cationic polymers, or cationic polyelectrolytes in “D.-J. Liaw, C.-C. Huang, W.-F. Lee, J. Borbely, E.-T. Kang, Synthesis and characteristics of the poly(carboxybetaine) s and the corresponding cationic polymers, J. Polym. Sci. A Polym. Chem. 35 (1997) 3527-3536.”, incorporated herein by reference. The above reference also provides synthesis details for synthesizing non-limiting examples of one or more stabilizing polymer as described herein.

Non-limiting examples of stabilizing polymers of the present disclosure that may be suitable for use, may include those described as poly(sulfobetaine) s or cationic polymers in “W.-F. Lee, C.-C. Tsai, Synthesis and solubility of the poly(sulfobetaine) s and the corresponding cationic polymers: 1. Synthesis and characterization of sulfobetaines and the corresponding cationic monomers by nuclear magnetic resonance spectra, Polymer. 35 (1994) 2210-2217.”, incorporated herein by reference. The above reference also provides synthesis details for synthesizing non-limiting examples of one or more stabilizing polymer as described herein.

Non-limiting examples of stabilizing polymers of the present disclosure that may be suitable for use, may include those described as poly(sulfobetaine methacrylate) (PSBMA) in “R. Lalani, L. Liu, Synthesis, characterization, and electrospinning of zwitterionic poly(sulfobetaine methacrylate), Polymer. 52 (2011) 5344-5354.”, incorporated herein by reference. The above reference also provides synthesis details for synthesizing non-limiting examples of one or more stabilizing polymer as described herein.

Non-limiting examples of stabilizing polymers of the present disclosure that may be suitable for use, may include those described as zwitterionic polymers, polyzwitterions, polybetaines, polymeric zwitterions, polycarboxybetaines, polysulfobetaines, polymeric phosphobetaines, or poly(phosphobetaine) s in “A. Laschewsky, Structures and Synthesis of Zwitterionic Polymers, Polymers. 6 (2014) 1544-1601.”, incorporated herein by reference. The above reference also provides synthesis details for synthesizing non-limiting examples of one or more stabilizing polymer as described herein.

Non-limiting examples of stabilizing polymers of the present disclosure that may be suitable for use, may include those described as polycarboxybetaine esters, cationic polycarboxybetaine esters, carboxybetaine ester polymers, polycarboxybetaines, or zwitterionic polycarboxybetaines in “Z. Zhang, G. Cheng, L. R. Carr, H. Vaisocherová, S. Chen, S. Jiang, The hydrolysis of cationic polycarboxybetaine esters to zwitterionic polycarboxybetaines with controlled properties, Biomaterials. 29 (2008) 4719-4725.”, incorporated herein by reference. The above reference also provides synthesis details for synthesizing non-limiting examples of one or more stabilizing polymer as described herein.

Non-limiting examples of stabilizing polymers of the present disclosure that may be suitable for use, may include those described as CBMA-ethyl ester polymers, pCBMA-ethyl ester, pCBMA-EE, or cationic CBMA ester polymer in “L. R. Carr, S. Jiang, Mediating high levels of gene transfer without cytotoxicity via hydrolytic cationic ester polymers, Biomaterials. 31 (2010) 4186-4193.”, incorporated herein by reference. The above reference also provides synthesis details for synthesizing non-limiting examples of one or more stabilizing polymer as described herein.

### Stabilizing Polymer Combinations and Blocks

Copolymers are known art, where monomeric units of two or more polymers are combined to produce a hybrid polymer with the two different monomeric units. Copolymers may be assembled with alternating monomeric units or block copolymers with two different polymeric pieces combined. Copolymers may also be assembled in various combinations with different repetitions of certain monomeric units or a combination of blocks and alternating monomers.

A monomer or monomeric unit is a singular portion of a polymer that when bonded together creates the continuous chain of a polymer (not including end groups). A monomer or monomeric unit may be a part of a homopolymer or a part of a copolymer.

The inventors have discovered that RNA stabilizing substances may comprise a stabilizing polymer substance that is a copolymer, such as block copolymers or alternating copolymers. In some embodiments the monomeric units of two or more stabilizing polymer substances may be combined to create a copolymer. In some embodiments the monomeric units of two or more stabilizing polymer substances may be combined with one or more additional polymers to create a copolymer.

In some embodiments a stabilizing polymer substance may be a homopolymer. In some embodiments a stabilizing polymer substance may be copolymer, such as an alternating copolymer or block copolymer.

In some embodiments a stabilizing polymer substance may comprise one or more pendant groups wherein the pendant groups may be the same or different. In some embodiments a stabilizing polymer substance may comprise two or more pendant groups wherein the pendant groups may be the same or different.

In some embodiments a stabilizing polymer substance may comprise multiple types of pendant groups. In some embodiments a stabilizing polymer substance may comprise 2-5, 2-4, or 2-3 different types of pendant groups (e.g. 2, 3, 4, or 5 types of pendant groups). In some embodiments a stabilizing polymer substance may comprise multiple types of monomeric units. In some embodiments a stabilizing polymer substance may comprise 2-5, 2-4, or 2-3 different types of monomeric units (e.g. 2, 3, 4, or 5 types of monomeric units).

In some embodiments one or more of the following monomeric units may be combined to form one or more stabilizing polymer substances comprising a copolymer:

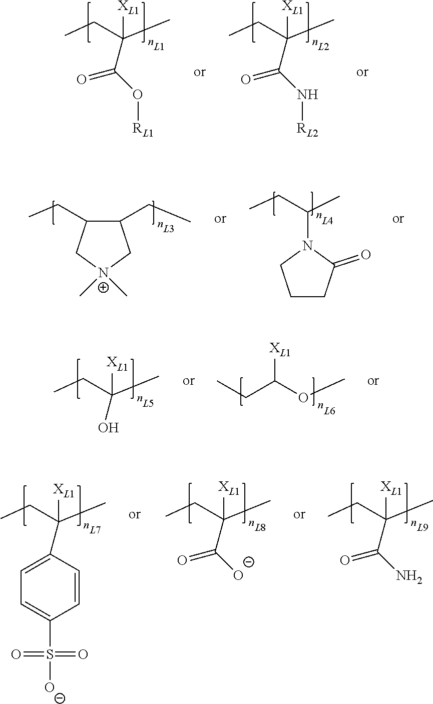

Wherein:
R_{L1} and R_{L2} are independent R_{L} groups independently selected at each occurrence as described herein this section;
X_{L1} is selected from H or CH₃;
n_{L1}-n_{L9} are independent integers selected from 1-10,000.

In some embodiments n_{L1}-n_{L9} may be selected from between about 1-500, 1-250, 1-100, or 1-50. In some specific embodiments n_{L1}-n_{L9} may be selected from between about 1-250, 1-100, or 1-50. In some even more specific embodiments n_{L1}-n_{L9} may be selected from between about 1-100, 1-50, or 1-20.

In some embodiments a stabilizing polymer substance may comprise a linear block copolymer. In some embodiments a stabilizing polymer substance may comprise a random block copolymer. In some embodiments a stabilizing polymer substance may comprise an alternating block copolymer.

In some embodiments a stabilizing polymer substance may comprise a block copolymer, wherein a stabilizing polymer substance may comprise a diblock polymer, triblock polymer, quaterblock polymer, or higher order multiblock polymer. In some embodiments a stabilizing polymer substance may comprise multiple types of blocks. In some embodiments a stabilizing polymer substance may comprise 2-5, 2-4, or 2-3 different types of blocks (e.g. 2, 3, 4, or 5 types of blocks).

In some embodiments a stabilizing polymer substance may comprise a copolymer, such as a bipolymer, terpolymer, or quaterpolymer, or higher order copolymer. In some embodiments a stabilizing polymer substance may comprise one or more the following types of copolymers, including, but not limited to: a linear copolymer, a random copolymer, an alternating copolymer, a statistical copolymer, a gradient copolymer, a periodic copolymer, a sequential copolymer, a block copolymer, a graft copolymer, a crosslinked copolymer, or a star copolymer, or combinations thereof.

In some embodiments a stabilizing polymer substance may comprise a block copolymer, wherein a stabilizing polymer substance may comprise a diblock polymer, triblock polymer, quaterblock polymer, or higher order multiblock polymer.

In some embodiments a stabilizing polymer substance may comprise a linear block copolymer. In some embodiments a stabilizing polymer substance may comprise a random block copolymer. In some embodiments a stabilizing polymer substance may comprise an alternating block copolymer.

In some embodiments an RNA stabilizing substance may comprise an anionic or polyanionic polymer. In some embodiments an RNA stabilizing substance may comprise a cationic or polycationic polymer. In some embodiments an RNA stabilizing substance may comprise a zwitterionic polymer.

In some embodiments a stabilizing polymer substance may be at least partially hydrolyzable. In some embodiments a stabilizing polymer substance may comprise one or more hydrolyzable bonds.

Embodiments of the present disclosure may include one or more stabilizing polymer substance with a molecular weight between about 1 kDa-100 kDa, or between about 1 kDa-50 kDa, or between about 5 kDa-50 kDa, as non-limiting examples (e.g about 1 kDa, 2.5 kDa, 5 kDa, 7.5 kDa, 10 kDa, 15 kDa, 20 kDa, 25 kDa, 30 kDa, 50 kDa, or 100 kDa, as non-limiting examples).

Embodiments comprising stabilizing polymer substances, may include one or more conjugate acid or conjugate base, or salt thereof.

In some embodiments an RNA stabilizing substance comprising a stabilizing polymer substance may be used in or to produce one or more RNA stabilizing composition described herein. In some embodiments an RNA stabilizing substance comprising a stabilizing polymer substance may be used in or to produce one or more RNA stabilizing composition described herein, where the concentration of a stabilizing polymer substance may be between about 0.01 mg/mL-100 mg/mL, or between about 0.01 mg/mL-50 mg/mL, or between about 0.05 mg/mL-50 mg/mL, or between about 0.1 mg/mL-50 mg/mL, or between about 0.1 mg/mL-20 mg/mL, or between about 0.1 mg/mL-10 mg/mL, or between about 0.5 mg/mL-10 mg/mL, as non-limiting examples. In some embodiments an RNA stabilizing substance comprising a stabilizing polymer substance may be used in or to produce one or more RNA stabilizing composition described herein, where the concentration of a stabilizing polymer substance may be less than about 50 mg/mL, or less than about 20 mg/mL, or less than about 10 mg/mL, or less than about 5 mg/mL, or less than about 1 mg/mL, as non-limiting examples. In some embodiments an RNA stabilizing substance comprising a stabilizing polymer substance may be used in or to produce one or more RNA stabilizing composition described herein, where the concentration of a stabilizing polymer substance may be at least about 0.01 mg/mL or greater, or at least about 0.05 mg/mL or greater, or at least about 0.1 mg/mL or greater, or at least about 0.5 mg/mL or greater, or at least about 1 mg/ml or greater, or at least about 5 mg/mL or greater, or at least 10 mg/mL or greater, or at least 20 mg/mL or greater, as non-limiting examples.

### Physical Properties

The viscosities described herein refer to the viscosity of the material at about 20° C. Viscosities may be values provided in reputable references. Example reputable reference sources are (1) handbooks and references published by CRC press, e.g., CRC Handbook of Chemistry and Physics, 95th Edition, Haynes, W M, ed., CRC Press, Boca Raton, FL, 2004, (2) primary references used for “Viscosity of Liquids” cited in editions of CRC Handbook of Chemistry and Physics.

Alternative to using reputable reference viscosity data, viscosity may be measured using cup and bob viscometer (or equivalent viscometer suitable for obtaining viscosity measurements at specified shear rates) at about 20° C. As a non-limiting example, cup and bob viscometer may be used at low shear rate (e.g., about 0.01/sec to about 0.1/sec). Measurements may be made using methods and procedures commonly used in the pharmaceutical industry.

Alternative to using reputable reference viscosity data for evaluating thixotropic properties of a substance, the viscosity may be measured. Measurements may be made using methods and procedures commonly used in the pharmaceutical industry. As a non-limiting example, measurements may be made at both a low shear rate (e.g., about 0.01/sec to about 0.1/sec) and using at least one shear rate substantially greater than the low shear rate (e.g., a shear rate at least 10 times or at least 100 times the low shear rate).

In some embodiments a composition comprising one or more RNA substance and one or more RNA stabilizing substance may be a single substance liquid or fluid or may be a combination of substances that comprise a liquid or fluid. As non-limiting examples, one or more composition described herein, may be a liquid, solution, fluid, syrup, emulsion, colloid, or suspension, and may also comprise one or more liquid or solid carriers. As non-limiting examples, one or more RNA stabilizing composition described herein may have a viscosity greater than about 3 centipoise (cP); or greater than about 5 cP; or greater than about 10 cP; or greater than about 15 cP; or greater than about 20 cP; or greater than about 30 cP; or greater than about 40 cP; or greater than about 50 cP; or greater than about 75 cP; or greater than about 100 cP; or greater than about 150 cP; or greater than about 200 cP; or greater than about 300 cP; or greater than about 400 cP; or greater than about 500 cP; or greater than about 750 cP; or greater than about 1,000 cP; or greater than about 10,000 cP; or greater than about 20,000 cP; or greater than about 50,000 cP; or greater than about 100,000 cP; or greater than about 200,000 cP; or greater than about 500,000 cP; or greater than about 1,000,000 cP.

In some embodiments a composition comprising one or more RNA substance and one or more RNA stabilizing substance may be a solid. As non-limiting examples, one or more composition described herein may be a pellet, powder, or tablet, and may also include solid carriers.

In some embodiments a composition comprising one or more RNA substance and one or more RNA stabilizing substance may be a gel comprising a liquid, such as water. As non-limiting examples, one or more composition described herein, may be a hydrated solid, or porous solid filled with or retaining water or other liquid or solution, and may also include solid or liquid carriers. In some embodiments an RNA stabilizing composition may be a gel or viscous fluid comprising water (e.g. a hydrogel or thixotropic fluid comprising water as non-limiting examples).

In some embodiments a composition comprising one or more RNA substance and one or more RNA stabilizing substance may be a vapor or aerosol. As non-limiting examples, one or more composition described herein may be a gas, vapor, or aerosol, or suspension of particles or droplets suspended in one or more gases (such as, but not limited to, air, nitrogen, oxygen, carbon dioxide, or anesthetic gas) and may also include liquid or solid carriers.

### Water Weight Percent

Compositions described herein comprising one or more RNA substance and one or more RNA stabilizing substance may comprise water.

Embodiments of the present disclosure may include one or more RNA stabilizing compositions described herein, wherein a composition may comprise a water weight percent between about X and Y, where X may be 10%, 15%, 20%, 25%, 30%, 35%, or 40%, and Y is greater than X, and Y may be 99%, 98%, 96% 95%, 90%, 80%, 70%, 60%, or 50%, as non-limiting examples X may be 10% and Y may be 95% and a composition may comprise a water weight percent between about 10%-95%, or X may be 30% and Y may be 70% and a composition may comprise a water weight percent between about 30%-70%.

Embodiments of the present disclosure may include one or more RNA stabilizing compositions described herein, wherein a composition may comprise a water weight percent of at least about 10% or greater, or at least about 15% or greater, or at least about 20% or greater, or at least about 25% or greater, or at least about 30% or greater, or at least about 40% or greater, or at least about 50% or greater, or at least about 60% or greater.

Embodiments of the present disclosure may include one or more RNA stabilizing compositions described herein, wherein a composition may comprise a water weight percent less than about 95%, less than about 90%, less than about 85%, less than about 80%, less than about 75%, less than about 70%, less than about 60%, or less than about 50%.

Embodiments of the present disclosure may include compositions comprising one or more RNA substance and one or more RNA stabilizing substance described herein, where the composition is a liquid at about 20° C. In some embodiments a composition comprising one or more RNA substance and one or more RNA stabilizing substance may be a liquid at about 0° C., or about 10° C., or about 20° C.

In some embodiments a composition comprising one or more RNA substance and one or more RNA stabilizing substance may be a gel or viscous fluid (e.g. a thixotropic fluid) at about 0° C., or about 10° C., or about 20° C., wherein the gel or viscous fluid may comprise water as a non-limiting example.

### RNA Stabilizing Substance Total Weight Percent

Compositions described herein comprising one or more RNA substance and one or more RNA stabilizing substance may include different total weight percentages of one or more RNA stabilizing substance. The following embodiments describing weight percentage of one or more RNA stabilizing substance, include the total weight percentage of all RNA stabilizing substances in a composition, as a non-limiting example if a composition comprises two RNA stabilizing substances and one substance is 15% by weight and the other is 50% by weight, then the total weight percentage of RNA stabilizing substances in a composition is 65%.

Embodiments of the present disclosure may include one or more RNA stabilizing compositions described herein, wherein a composition may comprise an RNA stabilizing substance total weight percent between about X and Y, where X may be 5%, 10%, 20%, 30%, or 40%, and Y is greater than X, and Y may be 99%, 98%, 96%, 95%, 90%, 80%, 70%, 60%, or 50%, as non-limiting examples X may be 10% and Y may be 70% and a composition may comprise an RNA stabilizing substance concentration between about 10%-70%, or X may be 30% and Y may be 60% and a composition may comprise an RNA stabilizing substance concentration between about 30%-60%.

Embodiments of the present disclosure may include one or more RNA stabilizing compositions described herein, wherein a composition may comprise an RNA stabilizing substance total weight percent greater than about 1%, or greater than about 2%, or greater than about 5%, or greater than about 10%, or greater than about 20%, or greater than about 30%, or greater than about 40%, or greater than about 50%, or greater than about 60%.

Embodiments of the present disclosure may include one or more RNA stabilizing compositions described herein, wherein a composition may comprise an RNA stabilizing substance total weight percent that is less than about 95%, or less than about 90%, or less than about 80%, or less than about 70%, or less than about 60%, or less than about 50%, or less than about 40%, or less than about 30%, or less than about 20%, or less than about 10%, or less than about 5%, or less than about 2%, or less than about 1%.

### Additive Substances

The inventors have surprisingly discovered that adding one or more additive substances may further improve RNA stability in one or more compositions comprising one or more RNA stabilizing substance and one or more RNA substance. These additive substances that may further improve RNA stability are herein referred to as additive substances. The inventors have surprisingly discovered that the addition of one or more additive substances to a composition comprising one or more RNA stabilizing substance and one or more RNA substance may further improve RNA stability.

Embodiments of the present disclosure comprising one or more RNA stabilizing substance and one or more RNA substance, may also comprise one or more additive substances. These embodiments comprising one or more additive substances may include one or more RNA stabilizing composition as described herein that may also comprise one or more additive substances.

Additive substances may include one or more buffer substance selected from the following (herein referred to as buffer additive substances): acetate, phosphate, citrate, or tris (also known as tris(hydroxymethyl) aminomethane or tromethamine). Embodiments of the present disclosure comprising one or more RNA stabilizing substance and one or more RNA substance, may also comprise one or more buffer additive substance. In some embodiments one or more RNA stabilizing composition described herein may comprise one or more buffer additive substance, wherein the pH of the composition may be between about 5-9, or between about 5-8, or between about 6-9, or between about 6-8, or between about 7-8.

Embodiments of the present disclosure may include one or more RNA stabilizing composition described herein, wherein the concentration of a buffer additive substance may be greater than about 5 mM, or greater than about 10 mM, or greater than about 20 mM, or greater than about 50 mM, or greater than about 100 mM, or greater than about 150 mM, or greater than about 200 mM, or greater than about 250 mM, or greater than about 500 mM.

Additive substances may include one or more inorganic cation substances selected from the following (herein referred to as inorganic cation additive substances): Li, Na, K, Cs, Ag, Au, Pt, Ti, Rb, N_{H4}, Mg, Mn, or Zn. Embodiments of the present disclosure comprising one or more RNA stabilizing substance and one or more RNA substance, may also comprise one or more inorganic cation additive substances. As a non-limiting example an inorganic cation additive substance may include one or more salts comprising one or more inorganic cation (including inorganic and organic salts comprising one or more inorganic cation).

Embodiments of the present disclosure may include one or more RNA stabilizing composition described herein, wherein the concentration of an inorganic cation additive substance may be greater than about 5 mM, or greater than about 10 mM, or greater than about 20 mM, or greater than about 50 mM, or greater than about 100 mM, or greater than about 150 mM, or greater than about 200 mM, or greater than about 250 mM, or greater than about 500 mM.

Additive substances may include sugars, sugar alcohols, polyols, or cyclitols, herein referred to as carbohydrate additive substances. Non-limiting examples of carbohydrate additive substances that may be used, may include one or more of the following: sucrose, glucose, fructose, trehalose, sorbitol, glycerol, mannitol, xylitol, maltose, dextrose, xylose, mannitol, maltitol, isomalt, xylitol, lactitol, lactose, erythritol, threitol, arabitol, ribitol, galactitol, fucitol, iditol, inositol, myo-inositol, volemitol, maltotriitol, maltotetraitol, polyglycitol, hydrogenated starch hydrolysates, rhamnose, ribose, mannose, galactose, fucose, arabinose, dextrin, erythrose, altrose, allose, lyxose, or combinations thereof.

Embodiments of the present disclosure may include one or more RNA stabilizing composition described herein, wherein the concentration of a carbohydrate additive substance may be between about 10 mM-4M, such as between about 100 mM-4M, 250 mM-4M, 500 mM-4M, 100 mM-2M, 250 mM-2M, 500 mM-2M, 100 mM-1M, 250 mM-1M, or 500 mM-1M. Embodiments of the present disclosure may include one or more RNA stabilizing compositions described herein, wherein a composition may comprise one or more carbohydrate additive substance with a concentration greater than 10 mM, or greater than 50 mM, or greater than 100 mM, or greater than 200 mM, or greater than 500 mM, or greater than 1M.

Additive substances may also include one or more of the following supplemental additive substances herein referred to as supplemental additive substances. Non-limiting examples of supplemental additive substances that may be used may include one or more of the following: acesulfame, saccharin, aspartame, hexylene glycol, creatine, creatine phosphate, thiamine, ectoine, pyridoxal 5′-phosphate, biotin, (D or L) pantothenic acid (e.g. pantothenate), taurine, N,N-dimethylphenethylamine, and benzyltriethylammonium.

Embodiments of the present disclosure may include one or more RNA stabilizing composition described herein, wherein the concentration of a supplemental additive substance may be between about 5 mM-2M, such as between about 10 mM-2M, 20 mM-2M, 50 mM-2M, 10 mM-1M, 20 mM-1M, 50 mM-1M, or 100 mM-1M. Embodiments of the present disclosure may include one or more RNA stabilizing compositions described herein, wherein a composition may comprise one or more supplemental additive substance with a concentration greater than 5 mM, or greater than 10 mM, or greater than 20 mM, or greater than 50 mM, or greater than 100 mM, or greater than 200 mM.

Additive substances may also include one or more of the following amino acid additive substances herein referred to as amino acid additive substances. Non-limiting examples of amino acid additive substances that may be used may include one or more of the following: glutamate, aspartate, glycine, proline, threonine, serine, alanine, valine, isoleucine, leucine, methionine, phenylalanine, tyrosine, or tryptophan.

Embodiments of the present disclosure may include one or more RNA stabilizing composition described herein, wherein the concentration of an amino acid additive substance may be between about 5 mM-1M, such as between about 10 mM-1M, 50 mM-1M, 5 mM-500 mM, 10 mM-500 mM, or 20 mM-500 mM. Embodiments of the present disclosure may include one or more RNA stabilizing compositions described herein, wherein a composition may comprise one or more amino acid additive substance with a concentration greater than 5 mM, or greater than 10 mM, or greater than 20 mM, or greater than 50 mM, or greater than 100 mM.

Embodiments comprising additive substances, may include one or more conjugate acid or conjugate base, stereoisomer, or salt thereof.

Embodiments of the present disclosure comprising one or more RNA stabilizing substance and one or more RNA substance, may comprise one or more excipient or diluent, such as one or more pharmaceutically acceptable excipient or diluent.

### Non-Limiting Example Multi-Component Compositions

The inventors have discovered that combinations of RNA stabilizing substances comprising compounds from more than one RNA stabilizing substance category may be synergistic and provide better RNA stability than either individual compound (e.g., uridine-5′-monophosphate, trimethylglycine (TMG), and pyridine-2,3-dicarboxylate as non-limiting examples). The inventors have discovered that the stability of RNA substances may be enhanced in compositions comprising an RNA substance and multiple RNA stabilizing substances. Non-limiting embodiments of the present disclosure include RNA stabilizing substances that may comprise one or more compounds that are members of stabilizing substance categories as described herein. Embodiments of the present disclosure may include one or more RNA stabilizing composition comprising one or more RNA substance and one or more RNA stabilizing substance, wherein one or more RNA stabilizing substance may be selected from one or more categories of RNA stabilizing substances described herein. As a non-limiting example an RNA stabilizing composition may comprise multiple RNA stabilizing substances, wherein the RNA stabilizing substances may be from the same or different categories of RNA stabilizing substances.

The following list includes non-limiting example multi-component compositions comprising at least one or more RNA substance and at least one or more RNA stabilizing substance. These compositions may also include one or more additional substance described herein (including but not limited, one or more additional RNA stabilizing substance, cellular uptake agent, or additive substance, as non-limiting examples)
1. Uridine-5′-monophosphate, TMG, and at least one RNA substance
2. Uridine-5′-monophosphate, TMG, pyridine-3,5-dicarboxylate, and at least one RNA substance.
3. Guanosine-5′-monophosphate, 5-oxo-proline (e.g. pyroglutamate), and at least one RNA substance.
4. 2′deoxy-guanosine-5′-monophosphate, 5-oxo-proline, and at least one RNA substance.
5. Xanthosine-5′-monophosphate, TMG, and at least one RNA substance.
6. Guanosine-5′-monophosphate, glutamate, and at least one RNA substance.
7. Guanosine-5′-monophosphate, N-acetyl-aspartate, and at least one RNA substance.
8. Xanthosine-5′-monophosphate, N-acetyl-glutamate, TMG and at least one RNA substance.
9. Inosine-5′-monophosphate, TMG, and at least one RNA substance
10. Inosine-5′-monophosphate, TMG, pyridine-3,5-dicarboxylate, and at least one RNA substance.
11. 2′-deoxy-guanosine-5′-monophosphate, N-acetyl-glutamate, and at least one RNA substance
12. TMG, pyridine-2,6-dicarboxylate, uridine-5′-monophosphate, sorbitol, and at least one RNA substance
13. Inosine-5′-monophosphate, N-acetyl proline, pyridine-3,5-dicarboxylate, and at least one RNA substance
14. Uridine-5′-monophosphate, pyridine-3,5-dicarboxyalate, TMG, and at least one RNA substance
15. 2′-deoxy-guanosine-5′-monophosphate, N-(4-amino benzoyl)-L-glutamate, and at least one RNA substance
16. Oritidine-5′-monophosphate, TMG, and at least one RNA substance
17. Inosine-5′-monophosphate, TMG, sorbitol and at least one RNA substance
18. Oritidine-5′-monophosphate, TMG, pyridine-3,5-dicarboxylate, and at least one RNA substance
19. Guanosine-5′-monophosphate, pyridine-3,5-dicarboxylate, and at least one RNA substance
20. Xanthosine-5′-monophosphate, TMG, prydine-3,5-dicarboxylate, and at least one RNA substance
21. Uridine-5′-monophosphate, pyridine-2,6-dicarboxylate, TMG, choline, and at least and at least one RNA substance.
22. Inosine-5′-monophosphate, N-acetyl-aspartate, carnitine, and at least one RNA substance.
23. Guanosine-5′-monophosphate, N-(4-amino benzoyl)-L-glutamate, and at least one RNA substance.
24. Uridine-5′-monophosphate, N-acetyl tyrosine, alpha-GPC, pyridine-3,5-dicarboxylate, choline, sorbitol and at least one RNA substance.
25. 2′-deoxy-guanosine-5′-monophosphate, N-acetyl-aspartate, and at least one RNA substance.
26. Uridine-5′-monophosphate, N-acetyl aspartate, TMG, sorbitol and at least one RNA substance.
27. 2′-deoxy-guanosine-5′-monophosphate, 5-oxo-proline, N-(4-amino benzoyl)-L-glutamate, and at least one RNA substance.

Embodiments of the present disclosure may include one or more RNA stabilizing compositions described herein, wherein the pH of a composition may be between about 5-9, or between about 5-8, or between about 5-7, or between about 6-9, or between about 6-8, or between about 6-7, or between about 7-9, or between about 7-8.

In some embodiments one or more RNA stabilizing composition described herein may also comprise one or more buffering agent or pharmaceutically acceptable buffering agent. Non-limiting examples of buffers or buffering agents may include one or more Good's buffers. As a non-limiting example, one or more buffers or buffering agents may include, but are not limited to: Tris, citrate, acetate, bis-tris, carbonate, bicarbonate, phosphate, imidazole, MES, ADA, ACES, PIPES, MOPSO, bis-tris propane, BES, MOPS, TES, HEPES, DIPSO, MOBS, TAPSO, HEPPSO, POPSO, TEA, EPPS, tricine, glycine, diglycine, bicine, HEPBS, TAPS, AMPD, TABS, AMPSO, CHES, CAPSO, AMP, CAPS, or CABS, or mixtures or combinations thereof.

### Cellular Uptake Agents

In some embodiments of the present disclosure an RNA stabilizing composition comprising one or more RNA stabilizing substance and one or more RNA substance may also comprise one or more substance to promote the RNA's ability to enter cells (herein referred to as cellular uptake agents), example substances being, including but not limited to, lipids, lipidoids, polymers, detergents, ionizable polymers, ionizable lipids, cationic polymers, cationic lipids, sterols, cationic detergents, ionizable detergents, lipid particles (including lipid nanoparticles (LNPs)), detergent micelles, micelles, liposomes, nanoliposomes, exosomes, or membrane vesicles. Examples of cell entry may include, but are not limited to, fusion with a cellular membrane, endocytosis, pinocytosis, phagocytosis, diffusion (e.g. passive diffusion or active diffusion), direct microinjection, electroporation, or similar mechanisms to deliver an RNA substance to a cell. Non-limiting examples of cells may include, eukaryotic cells, prokaryotic cells, plant cells, bacterial cells, fungal cells, insect cells, human cells, or cells of an organ, tissue, plant, animal, or vertebrate animal, including but not limited to cells of humans, mammals, or non-human primates.

As used herein, cellular uptake agents (also known as transfection agents or cell penetration agents) means substances that promote RNA's ability to enter cells (including a cell's cytoplasm), such as eukaryotic cells, prokaryotic cells, fungal cells, mammalian cells, animal cells, human cells, plant cells, bacterial cells, mycoplasma, or insect cells as non-limiting examples. As a non-limiting example, cellular uptake agents may also promote RNA's ability to enter the cytoplasm of a cell, such as by promoting endosomal escape of the RNA from an endosome after entering a cell.

Cellular uptake agents are known art when used with RNA and may also be referred to as gene delivery agents, transfection agents, cellular delivery agents, cell penetrating agents, or complexation agents. The present disclosure uses cellular uptake agents in the novel configuration of one or more cellular uptake agent with one or more RNA stabilizing substance and one or more RNA substance. In some embodiments, at least one or more cellular uptake agent may be combined with at least one or more RNA substance and at least one or more RNA stabilizing substance either in advance and stored together or stored separately, such as in a two-compartment chamber, and combined close to the time of administration. In some embodiments a composition comprising one or more RNA substance and one or more cellular uptake agent, may also comprise one or more RNA stabilizing substance, wherein one or more RNA stabilizing substance may improve RNA stability. In some embodiments an RNA stabilizing composition comprising one or more RNA substance and one or more cellular uptake agent, may also comprise one or more RNA stabilizing substance that may improve the stability of an RNA substance in conjunction with one or more cellular uptake agent.

Some embodiments of the present disclosure may include an RNA stabilizing composition comprising one or more RNA substance, one or more RNA stabilizing substance, and one or more cellular uptake agent. In some embodiments a composition comprising one or more RNA substance, one or more RNA stabilizing substance, and one or more cellular uptake agent, may comprise an RNA stabilizing substance that may improve the stability an RNA/cellular uptake agent particle or complex, such as a lipid particle (e.g. LNP) or polyplex comprising one or more RNA substance. As a non-limiting example, one or more composition described herein may comprise one or more RNA substance and one or more cellular uptake agent, wherein the cellular uptake agent may form a complex or particle comprising one or more RNA substance (such as by binding to an RNA substance through ionic interactions or at least partially encapsulating an RNA substance within a particle, such as a lipid particle or LNP, as non-limiting examples). In some embodiments an RNA stabilizing composition may comprise one or more RNA stabilizing substance that may improve the stability of a complex or particle (e.g. a lipid particle or LNP) comprising one or more cellular uptake agent and one or more RNA substance.

As a non-limiting example, a composition comprising one or more RNA stabilizing substance, one or more RNA substance and one or more cellular uptake agent may comprise an RNA stabilizing substance that may improve the stability of a lipid particle comprising an RNA substance by improving particle integrity during storage or help maintain a uniform particle size distribution.

As a non-limiting example, a composition comprising one or more RNA stabilizing substance, one or more RNA substance and one or more cellular uptake agent may comprise an RNA stabilizing substance that may improve the stability of an RNA substance when complexed with an ionizable polymer (e.g. a polyplex) or lipid particle by preventing RNA hydrolysis or degradation.

In some embodiments one or more RNA stabilizing composition described herein comprising one or more RNA substance, one or more RNA stabilizing substance, and one or more cellular uptake agent, may improve the stability of one or more particle or complex comprising an RNA substance and one or more cellular uptake agent. As a non-limiting example, a particle or complex comprising an RNA substance may include an RNA substance that is at least partially complexed with a polymer (such as an ionizable or cationic polymer, as non-limiting examples). As another non-limiting example, a particle or complex comprising an RNA substance may include an RNA substance that is at least partially encapsulated in or complexed with a lipid or lipid or lipid particle (such as an ionizable lipid, or cationic lipid, or LNP comprising an ionizable lipid or cationic lipid, as non-limiting examples).

In some embodiments one or more RNA stabilizing composition described herein may comprise one or more RNA stabilizing substance, one or more cellular uptake agent, and one or more RNA substance, wherein a particle or complex (e.g. a lipid particle or polyplex) comprising at least one or more cellular uptake agent and at least one or more RNA substance may have a specified size distribution where the mean largest outside perimeter of the particles or complex may be between about X and Y, where X may be 10 nm, 20 nm, 50 nm, 100 nm, 200 nm, or 400 nm, 1 μm, 2 μm, 5 μm, 10 μm, 20 μm, or 50 μm and Y is greater than X, and Y may be 1 mm, 500 μm, 200 μm, 100 μm, 50 μm, 20 μm, 10 μm, 5 μm, 2 μm, 1 μm, 500 nm, 400 nm, 300 nm, 200 nm, 100 nm, or 80 nm. As non-limiting examples X may be 50 nm and Y may be 300 nm and a composition may comprise a particle or complex with a size distribution where the mean largest outside perimeter of the particles or complex may be between about 50 nm-300 nm, or X may be 200 nm and Y may be 1 μm and a composition may comprise a particle or complex with a size distribution where the mean largest outside perimeter of the particles or complex may be between about 200 nm-1 μm.

In some embodiments one or more RNA stabilizing composition described herein may comprise one or more RNA stabilizing substance, one or more cellular uptake agent, and one or more RNA substance, wherein a particle or complex comprising at least one or more cellular uptake agent and at least one or more RNA substance may maintain a specified size distribution described herein for at least X period of time at a defined minimum temperature Y, where X may be at least 1 day, 2 days, 3 days, 4 days, 1 week, 2 weeks, 3 weeks, 1 month, 2 months, 3 months, 6 months, 12 months, 18 months, or 2 years, and Y may be at least −20° C., −10° C., 0° C., 4° C., 10° C., 15° C., 20° C., or 30° C. As non-limiting examples the specified size distribution of the particles or complex may be a mean largest outside perimeter between about 50 nm-300 nm and X may be at least 1 week and Y may be at least 20° C. and a composition may comprise a particle or complex that maintains a mean largest outside perimeter of between about 50 nm-300 nm for at least 1 week at a temperature of at least 20° C., or the specified size distribution of the particles or complex may be a mean largest outside perimeter between about 200 nm-1 μm and X may be at least 1 year and Y may be at least 4° C. and a composition may comprise a particle or complex that maintains a mean largest outside perimeter of between about 200 nm-1 μm for at least 1 year at a temperature of at least 4° C.

In some embodiments the mean perimeter of a particle or complex may be a hydrodynamic mean perimeter. As non-limiting examples, the mean perimeter may be measured with transmission electron microscopy (TEM), cryo-TEM, dynamic light scattering (DLS), or other known methods in the art.

In some embodiments one or more RNA stabilizing composition described herein may comprise one or more RNA stabilizing substance, one or more cellular uptake agent, and one or more RNA substance, wherein a particle or complex comprising at least one or more cellular uptake agent and at least one or more RNA substance may encapsulate or complex with at least X percent of RNA within the composition, where X may be at least 10%, 20%, 30%, 40%, 50%, 60%, 70%, 80%, or 85%. As non-limiting examples X may be 50% and a composition may comprise a complex or particle wherein at least 50% of an RNA substance within the composition may be complexed with or encapsulated in a particle, or X may be 70% and a composition may comprise a complex or particle wherein at least 70% of an RNA substance within the composition may be complexed with or encapsulated in a particle.

In some embodiments one or more RNA stabilizing composition described herein may comprise one or more RNA stabilizing substance, one or more cellular uptake agent, and one or more RNA substance, wherein a particle or complex (e.g. lipid particle or LNP, or polyplex or RNA/polymer complex) comprising at least one or more cellular uptake agent and at least one or more RNA substance may retain a specified minimum level of RNA encapsulated within a particle or RNA complexed to a particle for at least X period of time at a defined minimum temperature Y, where X may be at least 1 day, 2 days, 3 days, 4 days, 1 week, 2 weeks, 3 weeks, 1 month, 2 months, 3 months, 6 months, 12 months, 18 months, or 2 years, and Y may be at least −20° C., −10° C., 0° C., 4° C., 10° C., 15° C., 20° C., 30° C., 40° C., or 50° C. As non-limiting examples the specified minimum level of RNA remaining encapsulated or complexed may be at least 90% and X may be at least 4 days and Y may be at least 30° C. and a composition may comprise a particle or complex that retains at least 90% RNA remaining encapsulated or complexed for at least 4 days at a temperature of at least 30° C., or the specified minimum level of RNA remaining encapsulated or complexed may be about 70% and X may be at least 1 year and Y may be at least 10° C. and a composition may comprise a particle or complex that retains at least 70% RNA remaining encapsulated or complexed for at least 1 year at a temperature of at least 10° C. As a non-limiting example, the percentage of RNA that remains encapsulated or complexed is the ratio of encapsulated or complexed RNA to the total RNA content of a composition as measured by weight. As a non-limiting example, the RNA that remains encapsulated or complexed is divided by the total RNA content of the composition as measured by weight.

Measuring the percentage or quantity of RNA that is either encapsulated or complexed with cellular uptake agents is known art. As a non-limiting example, RNA encapsulation may be measured by measuring changes in fluorescence using a fluorescent dye, such as RiboGreen (Thermo Fisher Scientific, Waltham, MA) or equivalent that exhibits fluorescence when bound to RNA, but does not efficiently enter the LNPs or bind to the RNA when encapsulated or complexed with a cellular uptake agent; fluorescence measurements of either encapsulated or total RNA following treatment with a detergent (such as Triton-X 100 as a non-limiting example) to release encapsulated RNA, may be compared to determine the amount or percentage of encapsulated RNA vs total RNA in a composition (e.g. the ratio of encapsulated RNA compared to total RNA in a composition). As a non-limiting example, the amount of RNA complexed to one or more cellular uptake agents may be measured by comparing the amount of complexed RNA to the total amount of RNA in a composition following release of the RNA from the polymer complex using known methods, such as size exclusion chromatography, mass spectrometry, bioanalyzer, or other suitable method known in the art.

Non-limiting example methods of use of one or more RNA stabilizing composition described herein that may comprise one or more RNA stabilizing substance, one or more cellular uptake agent, and one or more RNA substance, may comprise a particle (e.g. lipid particle or LNP at least partially encapsulating or at least partially complexed with one or more RNA substance) or complex (e.g. a polyplex or RNA/polymer complex at least partially complexed with one or more RNA substance) comprising one or more cellular uptake agent and one or more RNA substance maintaining a specified size distribution for a defined minimum period of time at a defined minimum temperature as described herein.

Other non-limiting example methods of use of one or more RNA stabilizing composition described herein that may comprise one or more RNA stabilizing substance, one or more cellular uptake agent, and one or more RNA substance, may comprise a particle (e.g. lipid particle or LNP at least partially encapsulating or at least partially complexed with one or more RNA substance) or complex (e.g. a polyplex or RNA/polymer complex at least partially complexed with one or more RNA substance) comprising one or more cellular uptake agent and one or more RNA substance retaining a specified minimum level of RNA encapsulated within a particle or RNA complexed to a particle for defined minimum period of time at a defined minimum temperature as described herein.

As used herein a polyplex is a complex formed between a polymer (typically via electrostatic interactions, as a non-limiting example) and a nucleic acid (such as RNA or DNA) resulting in the formation of a particle (e.g., a nanoparticle) comprising a polymer/nucleic acid complex. As a non-limiting example an RNA substance may form a polyplex with a cationic polymer via electrostatic interactions between the cationic polymer and negatively charged RNA backbone to create a particle or nanoparticle.

Other non-limiting embodiments of the present disclosure may include one or more RNA stabilizing composition described herein comprising one or more RNA substance and one or more RNA stabilizing substance, wherein the composition may be substantially free of one or more cellular uptake agent (such as a lipid, lipidoid, or polymer as non-limiting examples). Non-limiting embodiments of the present disclosure may include one or more RNA stabilizing composition described herein comprising one or more RNA substance and one or more RNA stabilizing substance, wherein the composition may be substantially free of lipid or lipidoid. Other non-limiting embodiments of the present disclosure may include one or more RNA stabilizing composition described herein comprising one or more RNA substance and one or more RNA stabilizing substance, wherein the composition may be substantially free of an RNA substance encapsulated in a particle (such as a lipid particle or LNP as non-limiting examples) or substantially free of an RNA substance complexed with a particle (such a lipid, LNP, or polymer).

As used herein, substantially free of cellular uptake agent means that the ratio of the total amount of all RNA or RNA substances to the total amount of all cellular uptake agents (such as a lipid, lipidoid, or polymer as non-limiting examples) is at least 10:1 (e.g. 10×RNA: 1× Uptake Agent) as measured on a weight-by-weight basis. As a non-limiting example, an RNA stabilizing composition that is substantially free of cellular uptake agent may comprise a total RNA content (e.g. total weight) that is at least 10 times (e.g. 10×) greater than the total cellular uptake agent content (e.g. total weight of all lipid, lipidoid, or polymer, as non-limiting examples) as measured by weight. As another non-limiting example, an RNA stabilizing composition that is substantially free of cellular uptake agent may have a total RNA content of at least 1 mg and a total cellular uptake agent content of 0.1 mg or less.

In some embodiments the minimum ratio (e.g. RNA:cellular uptake agent) of the total amount of all RNA or RNA substances to the total amount of all cellular uptake agents (as measured on a weight-by-weight basis) in an RNA stabilizing composition may be at least equal to or greater than 1:10, 1:5, 1:2, 1:1, 2:1, 3:1, 4:1, 10:1, 20:1, 50:1, 100:1, 200:1, 500:1, or 1000:1; as a non-limiting example a ratio of 4:1 RNA to cellular uptake agent would mean that the total weight of RNA is at least equal to or greater than 4 times (e.g. 4×) the total weight of all cellular uptake agents (e.g. lipid, lipidoid, or polymer, as non-limiting examples) in a composition.

In some embodiments the minimum ratio (e.g. RNA:polymer) of the total amount of all RNA or RNA substances to the total amount of all cellular uptake agents comprising a polymer (as measured on a weight-by-weight basis) in an RNA stabilizing composition may be at least equal to or greater than 1:10, 1:5, 1:2, 1:1, 2:1, 3:1, 4:1, 10:1, 20:1, 50:1, 100:1, 200:1, 500:1, or 1000:1; as a non-limiting example a ratio of 10:1 RNA to cellular uptake agent polymer would mean that the total weight of RNA is at least equal to or greater than 10 times (e.g. 10×) the total weight of all cellular uptake agent polymers in a composition.

As used herein, substantially free of lipid or lipidoid means that the ratio of the total amount all RNA or RNA substances to the total amount of all lipids or lipidoids is at least 10:1 (e.g. 10×RNA: 1× Lipid) as measured on a weight-by-weight basis As a non-limiting example, an RNA stabilizing composition that is substantially free of lipid or lipidoid may comprise a total RNA content (e.g. total weight) that is at least 10 times (e.g. 10×) greater than the total lipid or lipidoid content (e.g. total weight) as measured by weight. As another non-limiting example an RNA stabilizing composition that is substantially free of lipid or lipidoid may have a total RNA content of at least 1 mg and a total lipid or lipidoid content of 0.1 mg or less.

In some embodiments the minimum ratio (e.g. RNA:lipid) of the total amount of all RNA or RNA substances to the total amount of all lipids or lipidoids (as measured on a weight-by-weight basis) in an RNA stabilizing composition may be at least equal to or greater than 1:10, 1:5, 1:2, 1:1, 2:1, 3:1, 4:1, 10:1, 20:1, 50:1, 100:1, 200:1, 500:1, or 1000:1; as a non-limiting example a ratio of 2:1 RNA to lipid would mean that the total weight of RNA is at least equal to or greater than 2 times (e.g. 2×) the total weight of all lipids or lipidoids in a composition.

As used herein, substantially free of encapsulated RNA or complexed RNA means that at least 50% of the RNA or RNA substance is free RNA that is not encapsulated inside of or complexed with a particle (e.g. lipid particle, LNP, or polymer as non-limiting examples).

In some embodiments an RNA stabilizing composition may comprise a minimum percentage of at least X percent of all RNA or RNA substances in a composition that may be free RNA that is not encapsulated inside of or complexed with a particle (such as a lipid particle, LNP, or polymer), wherein X may be 90%, 80%, 70%, 60%, 50%, 40%, 30%, or 20%. As a non-limiting example if X is 80% then at least 80% of all RNA or RNA substances in the composition may be free RNA that is not encapsulated inside of or complexed with particle (such as a lipid particle, LNP, or polymer).

In some embodiments of the present disclosure one or more method for providing an RNA stabilizing composition may comprise providing a composition to at least one of administer or deliver at least one or more RNA substance to at least one of a cell, organ, tissue, plant, animal, mammal, human, or subject in need thereof, wherein a composition may be substantially free of one or more cellular uptake agents (such as a lipid, lipidoid, or polymer as non-limiting examples). As a non-limiting example, a method may comprise providing one or more RNA stabilizing composition described herein to at least one of administer or deliver at least one or more RNA substance to at least one of a cell, organ, tissue, plant, animal, mammal, human, or subject in need thereof, wherein a composition may be substantially free of lipid or lipidoid.

In some non-limiting embodiments of the present disclosure one or more method for providing an RNA stabilizing composition may comprise providing a composition to at least one of administer or deliver at least one or more RNA substance to at least one of a cell, organ, tissue, plant, animal, mammal, human, or subject in need thereof, wherein a composition may comprise a defined minimum ratio (e.g. RNA:cellular uptake agent) of the total amount of all RNA or RNA substances to the total amount of one or more cellular uptake agents (as measured on a weight-by-weight basis) in an RNA stabilizing composition. As a non-limiting example, a method may comprise providing one or more RNA stabilizing composition described herein to at least one of administer or deliver at least one or more RNA substance to at least one of a cell, organ, tissue, plant, animal, mammal, human, or subject in need thereof, wherein a composition may comprise a defined minimum ratio (e.g. RNA:lipid) of the total amount of all RNA or RNA substances to the total amount of all lipids or lipidoids (as measured on a weight-by-weight basis) in an RNA stabilizing composition. As a non-limiting example an RNA stabilizing composition with a defined minimum ratio of the total amount of all RNA or RNA substances to the total amount of all lipids or lipidoids, may have an RNA:lipid ratio of at least equal to or greater than 2:1 wherein the total weight of RNA would be at least equal to or greater than 2 times (e.g. 2×) the total weight of all lipids or lipidoids in a composition. As a non-limiting example, a method may comprise providing one or more RNA stabilizing composition described herein to at least one of administer or deliver at least one or more RNA substance to at least one of a cell, organ, tissue, plant, animal, mammal, human, or subject in need thereof, wherein a composition may comprise a defined minimum ratio (e.g. RNA:polymer) of the total amount of all RNA or RNA substances to the total amount of all cellular uptake agents comprising a polymer (as measured on a weight-by-weight basis) in an RNA stabilizing composition.

In some non-limiting embodiments of the present disclosure one or more method for providing an RNA stabilizing composition may comprise providing a composition to at least one of administer or deliver at least one or more RNA substance to at least one of a cell, organ, tissue, plant, animal, mammal, human, or subject in need thereof, wherein a composition may be substantially free of at least one of encapsulated RNA or complexed RNA as described herein. As a non-limiting example, a method may comprise providing one or more RNA stabilizing composition described herein to at least one of administer or deliver at least one or more RNA substance to at least one of a cell, organ, tissue, plant, animal, mammal, human, or subject in need thereof, wherein a composition may comprise a defined minimum percentage of free RNA or free RNA substances that are not encapsulated inside of or complexed with a particle (such as a lipid, lipidoid, LNP, or polymer).

In other non-limiting embodiments of the present disclosure, one or more methods described herein may also comprise one or more RNA stabilizing composition described herein, wherein a composition may be at least one of a pharmaceutical composition, biostimulant composition, or implant. As a non-limiting example, a method may comprise providing one or more RNA stabilizing composition described herein to at least one of administer or deliver at least one or more RNA substance to at least one of a cell, organ, tissue, plant, animal, mammal, human, or subject in need thereof, wherein the composition may be a pharmaceutical composition or a biostimulant composition comprising a defined minimum ratio (e.g. RNA:lipid) of the total amount of all RNA or RNA substances to the total amount of all lipids or lipidoids (as measured on a weight-by-weight basis) in the composition.

Non-limiting embodiments of the present disclosure may provide methods for at least one of producing or providing one or more RNA stabilizing composition for administering or delivering at least one or more RNA substance to a cell, organ, tissue, plant, animal, mammal, human, or subject in need thereof, wherein a composition may be at least one of a pharmaceutical composition, biostimulant composition, or implant. As a non-limiting, one or more RNA stabilizing composition described herein may be used to produce at least one of a pharmaceutical composition (which may include prophylactic or diagnostic compositions, as non-limiting examples), biostimulant compositions, or implants as described herein. As a non-limiting example, a pharmaceutical composition may be administered to a subject using any suitable amount and any suitable route of administration which may be effective for preventing, treating, or diagnosing, a disease, disorder, or condition. As a non-limiting example, the exact amount may be selected to account for the characteristics of each particular subject, depending on factors such as, but not limited to, the species, age, and general condition of the subject, the severity of the disease, the particular composition, its mode of administration, its mode of activity, and the like.

Some embodiments of the present disclosure may include an RNA stabilizing composition comprising a combination or mixture of one or more RNA substance, one or more RNA stabilizing substance, and one or more cellular uptake agent.

Embodiments of the present disclosure that comprise one or more RNA substance, one or more RNA stabilizing substance, and one or more cellular uptake agent may include combining, such as by mixing, one or more RNA substance with one or more RNA stabilizing substance and one or more cellular uptake agent to produce an RNA stabilizing composition that also comprises a cellular uptake agent.

In some embodiments of the present disclosure an RNA stabilizing composition comprising one or more RNA substance, one or more RNA stabilizing substance, and one or more cellular uptake agent may be produced by combining, such as by mixing, at least one or more RNA substance, at least one or more RNA stabilizing substance, and at least one or more cellular uptake agent.

In some embodiments of the present disclosure an RNA stabilizing composition comprising a cellular uptake agent may comprise a combination or mixture of at least one or more RNA substance, at least one or more RNA stabilizing substance, and at least one or more cellular uptake agent.

### Environment and Degradation

As described in this disclosure, the stability of stored RNA substances may be improved by RNA substances being in storage environments comprising an RNA stabilizing substance. As non-limiting examples, the improved stability may occur when the storage environment has temperatures higher than an ultracold (as a non-limiting example, about −80° C.), cold (as a non-limiting example, about −20° C.), refrigerated (as a non-limiting example, about 4° C.) temperature, or warmer temperature. As a non-limiting example, the stability of an RNA substance may be determined by comparing the starting average molecular weight of a sample of the RNA substance to the average molecular weight of a sample of the RNA substance that has been stored for at least one predetermined time and temperature. The stability of an RNA substance may be determined by exposing the RNA substance to a specified temperature for a specified time duration and comparing the ending average molecular weight to the starting molecular weight to determine the amount of degradation. The amount of degradation of an RNA substance between the beginning and the end of a time interval is calculated using the formula D=[1−(AMWe/AMWs)]*100 where D is the amount of degradation, AMWe is the average molecular weight of the RNA substance at the end of the time interval, AMWs is the average molecular weight of the RNA substance at the start of the time interval with AMWe divided by AMWs with the quotient subtracted from 1 and the result multiplied by 100 for the result to be expressed as a percentage. For example, if the average molecular weight at the end of the time interval is the same as the average molecular weight at the start of the time interval the amount of degradation is zero (0) percent and if the average molecular weight at the end is one-half of the starting molecular weight then the amount of degradation is 50 percent and if the ending average molecular weight is 25 percent of the starting average molecular weight then the degradation is 75 percent. The rate of degradation for an RNA substance is the percent degradation divided by the duration of the time interval, for example 50 percent over 7 days or 50 percent per 7 days. As a non-limiting example, the time and temperature selected may be a predetermined duration at a defined temperature such as 48 hours at 40° C. The average molecular weight of an RNA substance may be determined by one or more means known in the art, including but not limited to, liquid chromatography (e.g. HPLC or FPLC), gel electrophoresis, mass spectrometry, bioanalyzer, size exclusion chromatography, or other means known in the art. The degradation of RNA substances may be determined using parameters other than direct molecular weight measurements by using parameters known to those skilled in the art that directly relate to molecular weight such as using apparent molecular weight determined using gel electrophoresis with reference markers indicating the size of a polynucleotide in references' and samples' lanes.

As non-limiting example embodiments of the present disclosure, a composition comprising at least one RNA substance and at least one RNA stabilizing substance may stabilize the RNA substance such that the amount of degradation (D) of the RNA substance (defined as D=[1−(AMWe/AMWs)]*100) is less than about X % with temperatures exceeding a defined minimum temperature of about T° C. where X may be less than about 5%, 10%, 15%, 20%, 25%, 30%, 35%, 40%, or 50%; and T may be about −80° C., −60° C., −40° C., −30° C., −20° C., −10° C., 0° C., 2° C., 4° C., 6° C., 8° C., 10° C., 15° C., 20° C., 25° C., 30° C., 40° C., or 50° C.; for at least one of about 1 hour, about 2 hours, about 4 hours, about 8 hours, about 12 hours, about 18 hours, about 24 hours, about 48 hours, about 72 hours, about 100 hours, about 7 days, about 14 days, about 30 days, about 60 days, about 3 months, about 6 months, about 12 months, about 18 months, or about 24 months. As non-limiting examples, the exposure to temperatures of at least the defined temperature may be continuous or the exposure may be intermittent. As a non-limiting example, if X is 10% and T is 20° C. a composition comprising at least one RNA substance and at least one RNA stabilizing substance may stabilize the RNA substance such that the amount of degradation (D) of the RNA substance is less than about 10% in an environment with temperatures exceeding a defined minimum temperature of about 20° C. for at least one of about 1 hour, about 2 hours, about 4 hours, about 8 hours, about 12 hours, about 18 hours, about 24 hours, about 48 hours, about 72 hours, about 100 hours, about 7 days, about 14 days, about 30 days, about 60 days, about 3 months, about 6 months, about 12 months, about 18 months or about 24 months. As non-limiting examples, the exposure to temperatures of at least the defined temperature may be continuous or the exposure may be intermittent. As a non-limiting example, a composition comprising at least one RNA substance and at least one RNA stabilizing substance may stabilize the RNA substance such that the amount of degradation (D) of the RNA substance is less than about 10% of RNA molecules in an environment with temperatures exceeding about 20° C. for about 3 months or about 6 months.

### Chambers

As used herein, chamber means an enclosed volume capable of containing at least one of a solid, powder, liquid, fluid, gel, aerosol, vapor, or gas that may be sealed or at least partially sealed and later allow at least some of its contents to be at least one of partially delivered, removed, emptied, dispensed, opened, accessed, or penetrated. As a non-limiting example, a chamber may include, but not limited to, bottles, containers, vials, tubes, trays, plates (e.g. 96-well plates or 384-well plates), jars, syringes (including prefilled syringes), blisters, capsules, tablets, cartridges, inhalers, packets, pods, bags, boxes, or other packages that may hold a solid, powder, liquid, fluid, gel, aerosol, or gas. As non-limiting examples, chambers may be single-piece gel soft capsules or may be two-piece gel hard capsules. As non-limiting examples, chambers may be capsules containing at least one RNA stabilizing substance and at least one RNA substance that may be used in an inhaler. As a non-limiting example, in some embodiments a chamber may comprise a moveable cover, wherein a moveable cover may allow access to at least some of the contents inside of the chamber or at least partially seal the chamber to prevent at least some of the contents from leaking, evaporating, or spilling out of the chamber. As non-limiting examples, a moveable cover may include a friction-fit cap, screw-cap, adhesive sheet, or lid that may be sealed and then opened or punctured (e.g. by a needle, pipette, or hollow tube) to access the contents inside of the chamber and may optionally be resealed to seal the contents inside the chamber. As a non-limiting example, one or more chambers may be stored inside of one or more additional chamber, such as tablet or capsule stored inside of a blister pack, bottle, or other container.

In some embodiments a chamber may be sealed, such as hermetically sealed or sealed with a resealable cover (e.g. a friction-fit cap, screw-cap, plug, or lid, as non-limiting examples). As a non-limiting example, a chamber may contain one or more composition, substance, or combination of substances described herein (e.g. an RNA stabilizing composition, RNA substances, RNA stabilizing substances, cellular uptake agents, additive substances, inorganic cations (or salts thereof), buffering agents, water, solvents, or one or more other substances described herein as non-limiting examples) wherein the chamber may be hermetically sealed or sealed with a resealable cover.

As a non-limiting example, a chamber may include a reservoir for placing or storing one or more substances or compositions described herein. As a non-limiting example, a chamber may include a single reservoir or multiple reservoirs, such as a multi-well reservoir, as non-limiting examples, (e.g. a 96-well plate, or 384-well plate, as non-limiting examples) wherein a multi-well reservoir may comprise a reservoir with multiple individual wells that may at least partially separate the contents within each individual well.

In some embodiments, chambers may contain one or more composition or substance described herein, such as one or more RNA stabilizing composition or one or more RNA stabilizing substance, or one or more additional substances (e.g. one or more buffering agents, water, solvents, cellular uptake agents, additive substances, inorganic cations (or salts thereof), or RNA substances, or one or more other substances described herein as non-limiting examples). As a non-limiting example, chambers may contain one or more substance described herein (e.g. an RNA stabilizing substance, RNA substance, cellular uptake agent, or additional substances as non-limiting examples) stored individually (such as in a kit as a non-limiting example) or may contain combinations of one or more substance described herein (e.g. combinations of one or more RNA stabilizing substance, RNA substance, cellular uptake agent, or additional substances as non-limiting examples). As a non-limiting example, chambers may contain combinations of multiple RNA stabilizing substances as described herein (e.g. two or more RNA stabilizing substances as a non-limiting example) or may contain combinations of one or more RNA stabilizing substance and one or more RNA substance, or one or more cellular uptake agent. As another non-limiting example, chambers may contain combinations of one or more RNA stabilizing substance and one or more additional substance (such as one or more buffering agents, water, solvents, cellular uptake agents, additive substances, salts, or RNA substances, or one or more other substances described herein as non-limiting examples).

As a non-limiting example, in some embodiments, when a chamber contains at least one or more RNA substance (such as in an RNA stabilizing composition) a chamber my contain substantially more than one nucleic acid molecule (e.g., may contain more than 100 molecules) as opposed to particles with a wall surrounding small amounts, (e.g. low count, approximately single, number of molecules) of nucleic acid. Chambers may have at least one outer dimension (e.g., diameter, length, width, depth, height, and so on) greater than the size of nanoparticles (nanoparticles typically have dimensions less than about 0.0005 mm) by having at least one outer dimension being at least about 0.001 mm. As non-limiting examples, chambers may have at least one outer dimension (e.g., diameter, length, width, depth, height, and so on) of at least 0.002 mm, or of at least 0.005 mm, or of at least 0.01 mm, or of at least 0.1 mm, or of at least 1 mm, or of at least 2 mm, or of at least 3 mm, or of at least 4 mm, or of at least 5 mm, or of at least 10 mm. As a non-limiting example, chambers containing at least one RNA stabilizing substance and at least one RNA substance for nasal spray-type administration may have dimensions in the approximate range of 0.02 mm to 0.12 mm. As a non-limiting example, for chambers containing at least one RNA stabilizing substance and at least one RNA substance for oral administration the length may be about 5 mm or larger, or the sum of length+width+depth may be between about 15 mm and about 50 mm, or the sum of length+width+depth may be less than about 25 mm, or the sum of length+width+depth may be between about 15 mm and 25 mm.

As non-limiting examples, chambers may contain more than 1,000 kg of an RNA stabilizing composition, or may contain up to 1,000 kg of an RNA stabilizing composition, or may contain up to 100 kg of an RNA stabilizing composition, or may contain up to 10 kg of an RNA stabilizing composition, or may contain up to 1 kg of an RNA stabilizing composition, or may contain up to 100 g of an RNA stabilizing composition, or up to 10 g of an RNA stabilizing composition, or up to 5 g of an RNA stabilizing composition, or up to 2 g of an RNA stabilizing composition, or up to 1 g of an RNA stabilizing composition.

As non-limiting example, chambers may contain one or more RNA stabilizing composition described herein, wherein a composition may be at least one of a pharmaceutical composition, biostimulant composition, or implant. As another non-limiting example, chambers may contain one or more composition, substance, or combination of substances described herein (e.g. an RNA stabilizing composition, RNA substances, RNA stabilizing substances, cellular uptake agents, additive substances, inorganic cations or salts thereof, buffering agents, water, solvents, or one or more other substances described herein as non-limiting examples).

As non-limiting examples, chambers may contain more than 1,000 kg of one or more composition, substance, or combination of substances described herein (e.g. an RNA stabilizing composition, RNA substances, RNA stabilizing substances, cellular uptake agents, additive substances, inorganic cations or salts thereof, buffering agents, water, solvents, or one or more other substances described herein as non-limiting examples); or may contain up to 1,000 kg of one or more composition, substance, or combination of substances described herein; or may contain up to 100 kg of one or more composition, substance, or combination of substances described herein; or may contain up to 10 kg of one or more composition, substance; or combination of substances described herein, or may contain up to 1 kg of one or more composition, substance, or combination of substances described herein; or may contain up to 100 g of one or more composition, substance, or combination of substances described herein; or may contain up to 10 g of one or more composition, substance, or combination of substances described herein; or may contain up to 5 g of one or more composition, substance, or combination of substances described herein; or may contain up to 2 g of one or more composition, substance, or combination of substances described herein; or may contain up to 1 g of one or more composition, substance, or combination of substances described herein. As a non-limiting example, chambers may contain at least 1 μg, or may contain at least 10 μg, or may contain at least 100 μg, or may contain at least 500 μg, or may contain at least 1 mg, or may contain at least 5 mg, or may contain at least 10 mg, or may contain at least 50 mg, or may contain at least 100 mg, or may contain at least 200 mg, or may contain at least 500 mg, or may contain at least 1 g, of one or more composition, substance, or combination of substances described herein.

As non-limiting examples, chambers may contain up to 100 L of one or more composition, substance, or combination of substances described herein (e.g. an RNA stabilizing composition, RNA substances, RNA stabilizing substances, cellular uptake agents, additive substances, salts, buffering agents, water, solvents, or one or more other substances described herein as non-limiting examples). As a non-limiting example, chambers may contain up to 100 L, or may contain up to 10 L, or may contain up to 1 L, or may contain up to 100 mL, or may contain up to 50 mL, or may contain up to 30 mL, or may contain up to 10 mL, or may contain up to 5 mL, or may contain up to 2.5 mL, or may contain up to 2 mL, or may contain up to 1 mL of one or more composition, substance, or combination of substances described herein. As a non-limiting example, chambers may contain at least 1 μL, or may contain at least 5 μL, or may contain at least 10 μL, or may contain at least 25 μL, or may contain at least 50 μL, or may contain at least 100 μL, of one or more composition, substance, or combination of substances described herein.

As a non-limiting example, a chamber may contain between about 1 μL-100 L of one or more composition, substance, or combination of substances described herein (e.g. an RNA stabilizing composition, RNA substances, RNA stabilizing substances, cellular uptake agents, additive substances, salts, buffering agents, water, solvents, or one or more other substances described herein as non-limiting examples). As a non-limiting example, chambers may contain between X and Y amount of one or more composition, substance, or combination of substances described herein, wherein X is less than Y, and X may be 1 μL, 5 μL, 10 μL, 25 μL, 50 μL, 100 μL, or 200 μL; and Y may be 100 L, 10 L, 1 L, 100 mL, 50 mL, 30 mL, 10 mL, 5 mL, 2.5 mL, 2 mL, or 1 mL. As a non-limiting example, X may be 1 μL and Y may be 1 L and a chamber may contain between about 1 μL-1 L of one or more composition, substance, or combination of substances described herein. As a non-limiting example, X may be 10 μL and Y may be 10 mL and a chamber may contain between about 10 μL-10 mL of one or more composition, substance, or combination of substances described herein.

As a non-limiting example, a chamber may contain between about 1 ng-10 kg of one or more composition, substance, or combination of substances described herein (e.g. an RNA stabilizing composition, RNA substances, RNA stabilizing substances, cellular uptake agents, additive substances, inorganic cations (or salts thereof), buffering agents, water, solvents, or one or more other substances described herein as non-limiting examples). As a non-limiting example, chambers may contain between X and Y amount of one or more composition, substance, or combination of substances described herein, wherein X is less than Y, and X may be Ing, 1 μg, 10 μg, 50 μg, 100 μg, 1 mg, 10 mg, 50 mg, 100 mg, 500 mg, 1 g, 10 g, 50 g, 100 g, or 1 kg; and Y may be 10 kg, 1 kg, 100 g, 50 g, 10 g, 5 g, 2 g, 1 g, 500 mg, 100 mg, 50 mg, 10 mg, 1 mg, or 100 μg. As a non-limiting example, X may be 10 μg and Y may be 1 g and a chamber may contain between about 10 μg-1 g of one or more composition, substance, or combination of substances described herein. As a non-limiting example, X may be 100 mg and Y may be 100 g and a chamber may contain between about 100 mg-100 g of one or more composition, substance, or combination of substances described herein.

In some embodiments of the present disclosure a chamber may contain one or more RNA substance with one or more RNA stabilizing substance. In some embodiments of the present disclosure a chamber may contain one or more RNA substance with two or more RNA stabilizing substances, or three or more RNA stabilizing substances. In some embodiments the chamber may be a vial. In some embodiments the chamber may be a prefilled syringe. In some embodiments the chamber may be a multi-compartment syringe. In some embodiments the chamber may be a capsule or tablet.

In other non-limiting embodiments, a chamber may contain one or more RNA stabilizing composition described herein, wherein a composition may be at least one of a pharmaceutical composition, biostimulant composition, or implant. As a non-limiting example, a chamber may be a capsule, tablet, vial, syringe, multi-compartment syringe, or embedded complex, containing one or more RNA stabilizing composition described herein.

In some embodiments of the present disclosure, each component included in an RNA stabilizing composition comprising one or more RNA substance and one or more RNA stabilizing substance, or optionally comprising one or more cellular uptake agent, may be stored separately, such as in a kit, or such as individually or as mixtures of one or more substance, and then combined later to produce a composition comprising one or more RNA substance and one or more RNA stabilizing substance, or one or more cellular uptake agent.

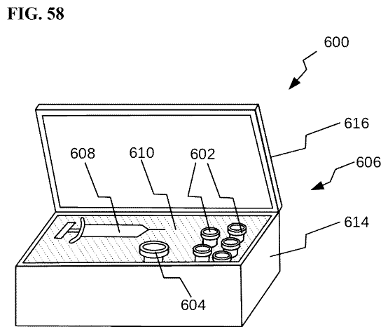

An example of such a kit 600 is shown in FIG. 58. As shown, the kit 600 includes one or more component vials 602 and one or more mixing/dispensing vials 604 contained in a package 606, such as a hinged box. Each of the component vials 602 may contain one or more components of the composition as described herein. For example, each component may be provided in a separate vial 602 or certain compatible components may be combined in a single vial 602 with other components provided individually or in combination in other vials 602.

The components can then be mixed in mixing/dispensing vial 604, for example, immediately prior to use. In this regard, the components from the vials 602 may be transferred into the vial 604, e.g., poured or injected into the vial 604 using a syringe 608, and then shaken or otherwise mixed. The components in the vials 602 may be premeasured and may provide a single unit or dose of the composition or multiple units/doses. Alternatively, the components from the vials 602 may be measured and combined by skilled workers. Optionally, e.g., in the case of vaccines, one or more syringes 608 may be provided in the kit 600 for drawing the composition from the vial 604 and administering to subjects. The vials 602 and 604 and syringes 608 may be secured by packing material 610 such as a foam material.

As described herein, many compositions, components, or combinations of components in accordance with the present disclosure can be stored without requiring extremely low temperatures. In cases where cold storage is required, the kit 600 can be transported in a cold storage unit or cold storage vehicles. The packaging 606 may be formed from materials suitable to withstand such cold storage such as various plastics or metals. In such cases, the vials 602, 604 and syringes 608 (if provided in the kit 600) may be formed from materials selected to withstand cold storage.

Although the kit is shown as including vials 602, 604 and syringes 608 for purposes of illustration, it will be appreciated that the components may be provided in other forms, e.g., non-liquid forms, and the composition may be provided for purposes other than vaccination. Accordingly, while a kit including some or all of the components of a composition in accordance with the present disclosure is useful and convenient, the nature of the kit can vary from the kit shown.

As a non-limiting example, vials 602 may comprise a concentrated composition comprising at least one RNA substance and at least one RNA stabilizing substance that after being mixed with at least one diluent is suitable for use, such as suitable for injection. In this embodiment the diluent may or may not be part of kit 600.

In another embodiment packaging 606 may comprise vials 602 that may be ready for use and packaging 606 provides a package for uses comprising at least one of as storage and transport and maintaining a desired environment for vials 602. Packaging 606 may comprise a cooling pack (not shown) that at least partly offsets thermal energy transferred from the storage and transport environment to the inside of package 606 where at least some of vials 602 are desired to experience a maximum target temperature.

In some embodiments kit 600 may comprise a cooling substance (not shown) that maintains the temperature of at least one of vials 602 at no more than a maximum target temperature. As a non-limiting example, the maximum target temperature may be about 4° C. or less. As a non-limiting example, the maximum target temperature may be about 20° C. or less. As a non-limiting example, a cooling substance that maintains the maximum target temperature or less may undergo a phase change to maintain the maximum target temperature. Non-limiting examples of cooling substances that may be used to maintain the maximum target temperature are solid phase water that may change to liquid phase water, solid phase carbon dioxide that may change phase to vapor phase carbon dioxide, or liquid phase nitrogen that may change from liquid phase to vapor phase nitrogen.

A non-limiting example embodiment of kit 600 comprises component vials 602 that may contain one or more components of the composition as described herein in which at least one component is an RNA substance and at least one component is an RNA stabilizing substance and a substance that maintains the maximum target temperature of at least one of the vials 602 to about 4° C. or less for at least one hour. In a non-limiting embodiment, the kit may maintain the temperature of at least one vial at about 20° C. or less for at least 24 hours. As another non-limiting embodiment, kit 600 may maintain the temperature of at least one vial at about 20° C. or less for at least 60 hours.

In another embodiment at least one or more cellular uptake agent may be combined with at least one or more RNA substance and at least one or more RNA stabilizing substance either in advance and stored together or stored separately, such as in a two-compartment chamber, and combined close to the time of administration.

### Non-Limiting Example Cellular Uptake Agents

In some embodiments a cellular uptake agent may comprise, but is not limited to, at least one or more of the following: lipids, lipidoids, polymers, zwitterionic polymers, zwitterionic lipids, ionizable polymers, ionizable lipids, cationic polymers, cationic lipids, neutral lipids, polymer-conjugated lipids (including PEG-lipids), sterols (including sterol analogs), detergents, cationic detergents, zwitterionic detergents, ionizable detergents, non-ionic detergents, polymer-conjugated detergents, detergent micelles, polyamines (including polyethylenimine (PEI)), dendrimers (including ionizable, cationic, or zwitterionic dendrimers), nanoparticles, lipid particles (including lipid nanoparticles (LNPs)), micelles, liposomes, nanoliposomes, lipoparticles, nanolipoparticles, exosomes, or membrane vesicles, or combinations or mixtures thereof.

As a non-limiting example, one or more cellular uptake agent may be at least part of a particle comprising at least one RNA substance. As a non-limiting example, one or more cellular uptake agent may at least partially surround or at least partially encapsulate at least one RNA substance. As another non-limiting example, one or more cellular uptake agent may be at least part of a particle comprising one of a hydrophilic or a hydrophobic core.

In some non-limiting example embodiments, a cellular uptake agent may comprise a lipid, polymer, or detergent, or combinations or mixtures thereof.

Non-limiting examples of cellular uptake agents comprising lipids may include: lipidoids, ionizable lipids, polymer-conjugated lipids (including PEG-lipids), neutral lipids (including non-ionic and zwitterionic lipids), cationic lipids, or combinations thereof.

Non-limiting examples of cellular uptake agents comprising detergents may include: ionizable detergents, polymer-conjugated detergents (including PEG-detergents), neutral detergents (including non-ionic and zwitterionic detergents), cationic detergents, or combinations thereof.

Non-limiting examples of cellular uptake agents comprising polymers may include: ionizable polymers (including polyamines such as polyethylenimine, as a non-limiting example), lipid-conjugated polymers, lipidoid-conjugated polymers, zwitterionic polymers, cationic polymers, dendrimers (including ionizable, cationic, or zwitterionic dendrimers), or combinations thereof.

In some embodiments a cellular uptake agent (such as a lipid particle) may comprise a micelle, bilayer, or multilamellar vesicle.

As used herein lipid particles include lipid nanoparticles, liposomes, nanoliposomes, lipoparticles, nanolipoparticles, exosomes, and membrane vesicles.

Non-limiting examples of cellular uptake agents comprising a lipid particle may include: lipid nanoparticles, liposomes, nanoliposomes, lipoparticles, nanolipoparticles, exosomes, or membrane vesicles.

As used herein, PEG is polyethylene glycol.

As used herein, a PEG lipid is a lipid modified with or conjugated to polyethylene glycol (referred to as a PEG-conjugated lipid or PEG-modified lipid).

Non-limiting example polymer conjugated lipids may include: PEG-conjugated lipids, cationic-polymer-conjugated lipids, PEG-conjugated dialkyloxypropyls, PEG-conjugated phospholipids, PEG-conjugated phosphatidylethanolamines, PEG-conjugated phosphatidic acids, PEG-conjugated ceramides, PEG-conjugated dialkylamines, PEG-conjugated diacylglycerols, or PEG-conjugated dialkylglycerols, or PEG-conjugated sterols, or combinations thereof.

Non-limiting example polymers may include cationic or ionizable polymers, cationic or ionizable polysaccharides, such as chitosan, polybrene, or polyethyleneimine (PEI), or derivatives or combinations thereof.

Other non-limiting example cellular uptake agents may include: cationic or ionizable peptides or proteins, cell penetrating peptides, basic polypeptides, cationic or ionizable dendrimers, polyamines, polyamine polysaccharides, amino polysaccharides, oligofectamine, modified polyaminoacids, β-aminoacid-polymers, modified polyethylenes, modified acrylates, modified amidoamines, modified polybetaaminoester, dendrimers, polypropylamine dendrimers, poly(amidoamine) PAMAM based dendrimers, polyimines, poly(ethyleneimine), poly(propyleneimine), polyallylamine, polylysine, polyornithine, poly/lysine/ornithine, poly(propylene imine), poly(vinyl amine), poly(2-aminoethyl methacrylate), cationic or ionizable carbohydrate backbone based polymers, cationic or ionizable cyclodextrin based polymers, cationic or ionizable dextrin or dextran based polymers, chitosan, or cationic or ionizable silane backbone based polymers, or combinations thereof.

Non-limiting examples of one or more cellular uptake agents that may be suitable for use with one or more RNA stabilizing composition of the present disclosure are described in US Patent Application Pub. No. US 2020/0383922 A1, incorporated herein by reference. Non-limiting examples may include, cationic or polycationic compounds that may be used as transfection or complexation agents.

Non-limiting examples of one or more cellular uptake agents that may be suitable for use with one or more RNA stabilizing composition of the present disclosure are described in U.S. Pat. No. 10,702,600, incorporated herein by reference. Non-limiting examples may include, nanoparticle formulations, cationic lipid nanoparticles, nanoparticles, liposomes, lipoplexes, lipid nanoparticles (LNPs), lipids, cationic lipids, ionizable lipids, PEG lipids, structural lipids, neutral lipids, non-cationic lipids, polymer-vitamin conjugate, block co-polymer or tri-block co-polymer, surface altering agents, or cationic or polycationic compounds.

Non-limiting examples of one or more cellular uptake agents that may be suitable for use with one or more RNA stabilizing composition of the present disclosure are described in U.S. Pat. No. 8,058,069, incorporated herein by reference. Non-limiting examples may include, lipid particles, stable nucleic acid-lipid particle (SNALP), lipids, lipid conjugates, amphipathic lipids, neutral lipids, non-cationic lipids, anionic lipids, cationic lipids, hydrophobic lipids, or sterols.

Non-limiting examples of one or more cellular uptake agents that may be suitable for use with one or more RNA stabilizing composition of the present disclosure are described in WO Patent Application Pub. No. WO 2021/156267 A1, incorporated herein by reference. Non-limiting examples may include, polymeric carriers, lipidoids or cationic lipidoids, lipid nanoparticles (LNPs), liposomes, lipoplexes, nanoliposomes, lipids, cationic or polycationic lipids, neutral lipids, ionizable lipids, polymer conjugated lipids, cationic or polycationic compounds, cationic or polycationic polymers, cationic or polycationic polysaccharides, cationic or polycationic proteins, or cationic or polycationic peptides.

Non-limiting examples of one or more cellular uptake agents that may be suitable for use with one or more RNA stabilizing composition of the present disclosure are described in U.S. Pat. No. 8,367,628, incorporated herein by reference. Non-limiting examples may include, lipids, amphoteric lipids, amphoteric liposomes, amphoteric liposomal mixtures, liposomal mixtures, sterols, cationic lipids, chargeable cationic lipids, chargeable anionic lipids, stable anionic lipids, neutral lipids, or mixtures of lipid components with amphoteric properties.

Non-limiting examples of one or more cellular uptake agents that may be suitable for use with one or more RNA stabilizing composition of the present disclosure are described in US Patent Application Pub. No. US 2021/0260097 A1, incorporated herein by reference. Non-limiting examples may include, nanoparticles, lipid nanoparticles (LNPs), lipids, cationic or ionizable lipids, anionic lipids, neutral lipids, amphipathic lipids, PEGylated lipids, or structural lipids.

Non-limiting examples of one or more cellular uptake agents that may be suitable for use with one or more RNA stabilizing composition of the present disclosure are described in US Patent Application Pub. No. US 2021/0261627 A1, incorporated herein by reference. Non-limiting examples may include, cationic or polycationic compounds, polymeric carriers, cationic polysaccharides, cationic lipids, polymers, cationic or polycationic polymers, copolymers, blockpolymers, or cationic or polycationic proteins or peptides, which may be used as transfection or complexation agents.

As used herein, the term lipid includes lipids, lipidoids, and lipidoid compounds.

As used herein, a cationic lipid is a lipid that carries a net positive charge at at least one pH value between about pH 4-9. As used herein, an ionizable lipid is a lipid that is capable of becoming ionized (e.g. protonated) at at least one pH value between about pH 4-7. As a non-limiting example, an ionizable lipid may carry a net neutral charge at a pH of about 7.4 and may become ionized (e.g. protonated) and carry a net positive charge at a pH of about 5 following ionization.

As non-limiting examples, an ionizable lipid may comprise an amine lipid, imidazole lipid, piperidine lipid, piperazine lipid, or pyrrolidine lipid, wherein an ionizable lipid may comprise one or more ionizable moieties comprising an amine, imidazole, piperidine, piperazine, or pyrrolidine group, or combinations thereof.

In some embodiments an RNA stabilizing composition comprising a cellular uptake agent may comprise a combination or mixture of one or more lipids or sterols including a cationic or ionizable lipid, a neutral lipid, a sterol (including sterol analogs), or a polymer-conjugated lipid (e.g. a PEG-lipid as a non-limiting example).

As a non-limiting example an RNA stabilizing composition comprising a cellular uptake agent may comprise a lipid particle (such as an LNP) comprising a combination of one or more lipids or sterols (including sterol analogs).

As another non-limiting example, an RNA stabilizing composition comprising a cellular uptake agent may comprise a lipid particle (such as an LNP), wherein the lipid particle may comprise one or more of the following molar ratios of lipids, or sterols (including sterol analogs): 20-60% cationic or ionizable lipid: 5-25% neutral lipid: 25-55% sterol: 0.5-15% polymer conjugated lipid, wherein the total equals 100%. As a non-limiting example, a lipid particle (e.g. an LNP) may have a molar ratio of ionizable lipid:neutral lipid:sterol:polymer lipid of about 50:10:38.5:1.5.

As another non-limiting example, an RNA stabilizing composition comprising a cellular uptake agent may comprise a lipid particle (such as an LNP), wherein the lipid particle may comprise a total molar content of cationic or ionizable lipid of at least 10%, or at least 20%, or at least 30%, or at least 40%. As another non-limiting example, an RNA stabilizing composition comprising a cellular uptake agent may comprise a lipid particle (such as an LNP), wherein the lipid particle may comprise a total molar content of cationic or ionizable lipid of less than 80%, or less than 70%, or less than 60%, or less than 50%, or less than 40%, or less than 30%, or less than 20%, or less than 10%.

N:P ratios of cellular uptake agents to nucleic acid is known in the art. The N:P ratio is the molar ratio between the nitrogen atoms on a cellular uptake agent (such as a cationic lipid) and the phosphate groups on a nucleic acid (such as an RNA molecule).

As a non-limiting example, an RNA stabilizing composition comprising a cellular uptake agent may comprise an N:P ratio wherein the molar ratio of cellular uptake agent nitrogens to RNA substance phosphate groups may be at least 0.25:1, or at least 0.5:1, or at least 0.75:1, or at least 1:1, or at least 1.5:1, or at least 2:1, or at least 3:1, or at least 4:1. As a non-limiting example, an RNA stabilizing composition may comprise an RNA substance and a cationic or ionizable lipid wherein the N:P ratio may be at least 1:1 (e.g. at least 1 lipid nitrogen for each RNA phosphate).

As another non-limiting example, an RNA stabilizing composition comprising a cellular uptake agent may comprise an N:P ratio wherein the molar ratio of cellular uptake agent nitrogens to RNA substance phosphate groups may be less than 10:1, or less than 8:1, or less than 6:1, or less than 4:1, or less than 2:1, or less than 1.5:1, or less than 1:1. As a non-limiting example, an RNA stabilizing composition may comprise an RNA substance and a cationic or ionizable lipid wherein the N:P ratio may be less than 2:1 (e.g. less than 2 lipid nitrogens for each RNA phosphate).

In some embodiments a cellular uptake agent may comprise one or more sterol (including sterol analogs). As non-limiting examples, one or more sterol or sterol analogs, may include: cholesterol, fecosterol, sitosterol (e.g. beta-sitosterol), ergosterol, campesterol, stigmasterol, brassicasterol, tomatidine, tomatine, ursolic acid, alpha-tocopherol, corticosteroids, prednisolone, dexamethasone, prednisone, or hydrocortisone, or combinations thereof.

In some embodiments, a cellular uptake agent may comprise a lipid wherein a lipid may comprise a polymer-conjugated lipid. As a non-limiting example, a polymer-conjugated lipid may comprise a polymer attached to a lipid (such as PEG attached to a lipid in a PEG-conjugated lipid).

Non-limiting examples of one or more polymer-conjugated lipid may include: 2 [(polyethylene glycol)-2000]—N,N-ditetradecylacetamide (ALC-0159), 1,2-dimyristoyl-glycero-3-methoxypolyethylene glycol (PEG-DMG), (such as DMG with PEG average molecular weight 2000, or 1,2-dimyristoyl-glycero-3-methoxypolyethylene glycol-2000 (DMG-PEG2000)), PEG-conjugated phosphatidylethanolamine, PEG-conjugated phosphatidic acid, PEG-conjugated ceramides (e.g. PEG-CerC14 or PEG-CerC20), PEG-conjugated dialkylamines, PEG-conjugated diacylglycerols, PEG-conjugated dialkylglycerols, N-[(methoxy polyethylene glycol) 2000) carbamyl]-1,2-dimyristyloxlpropyl-3-amine (PEG-c-DMA), R-3-[(ω-methoxy-poly(ethylene glycol) 2000) carbamoyl]-1,2-dimyristyloxlpropyl-3-amine (PEG-c-DOMG), a PEGylated diacylglycerol (PEG-DAG) [such as 1-(monomethoxy-polyethyleneglycol)-2,3-dimyristoylglycerol (PEG-DMG), as a non-limiting example], a PEGylated phosphatidylethanoloamine (PEG-PE), a PEG succinate diacylglycerol (PEG-S-DAG) [such as 4-0-(2′,3′-di(tetradecanoyloxy) propyl-1-O—(@-methoxy (polyethoxy)ethyl) butanedioate (PEG-S-DMG), as a non-limiting example], a PEGylated ceramide (PEG-cer), or a PEG dialkoxypropylcarbamate [such as @-methoxy (polyethoxy)ethyl-N-(2,3di(tetradecanoxy) propyl) carbamate or 2,3-di(tetradecanoxy) propyl-N—(@-methoxy (polyethoxy)ethyl) carbamate, as non-limiting examples], or combinations thereof.

Non-limiting examples of one or more polymer-conjugated lipid may comprise one or more hydrophilic polymers wherein one or more hydrophilic polymer may be attached to a lipid. As non-limiting examples, one or more polymer-conjugated lipids may comprise one or more of the following polymers attached to a lipid, including: polyvinylpyrrolidone, polymethyloxazoline, polyethyloxazoline, polyhydroxypropyl methacrylamide, polymethacrylamide or polydimethylacrylamide, polylactic acid, polyglycolic acid, or substituted celluloses such as hydroxymethylcellulose or hydroxyethylcellulose, or combinations thereof.

In some embodiments, a cellular uptake agent may comprise a lipid wherein a lipid may comprise a neutral lipid.

As a non-limiting example a neutral lipid may comprise one or more of the following groups or lipids including: phospholipids, phosphatidylcholines (including diacylphosphatidylcholines), phosphatidylethanolamines (including diacylphosphatidylethanolamines), ceramides (including dihydro ceramides), sphingomyelins (including dihydro sphingomyelins), cerebrosides, distearoylphosphatidylcholine (DSPC), dioleoylphosphatidylcholine (DOPC), dipalmitoylphosphatidylcholine (DPPC), dioleoyl-phosphatidylethanolamine (DOPE), palmitoyloleoylphosphatidylcholine (POPC), palmitoyloleoyl-phosphatidylethanolamine (POPE), 1,2-distearoyl-sn-glycero-3-phosphatidylcholine (1,2-DSPC), dioleoyl-phosphatidylethanolamine 4-(N-maleimidomethyl)-cyclohexane-1-carboxylate, 1,2-dioleoyl-sn-glycero-3-phosphoethanolamine (DOPE)-maleimide, N-(3-Maleimide-1-oxopropyl)-L-α-phosphatidylethanolamine, dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanolamine (SOPE), or 1,2-dielaidoyl-sn-glycero-3-phophoethanolamine (transDOPE), or combinations thereof.

In some embodiments, a cellular uptake agent may comprise a lipid wherein a lipid may comprise a cationic or ionizable lipid.

Non-limiting examples of cationic or ionizable lipids may include one or more of the following: (4-hydroxybutyl) azanediyl)bis(hexane-6,1-diyl)bis(2-hexyldecanoate) (ALC-0315), heptadecan-9-yl 8-((2-hydroxyethyl) (6-oxo-6-(undecyloxy) hexyl)amino) octanoate (SM-102), 9Z,12Z-octadecadienoic acid, 3-[4,4-bis(octyloxy)-1-oxobutoxy]-2-[[[[3-(diethylamino) propoxy|carbonyl]oxy]methyl]propyl ester (LP-01), bis(2-butyloctyl) 10-(N-(3-(dimethylamino)propyl) nonanamido) nonadecanedioate (Lipid A9), N,N,N-trimethyl-2,3-bis [(9Z)-1-oxo-9-octadecenyl]oxy]-1-propanaminium (1,2-dioleoyl-3-trimethylammoniumpropane), 5-(dimethylamino)-pentanoic acid, (6Z)-1,2-di-(4Z)-4-decen-1-yl-6-dodecen-1-yl ester (Lipid CL1), 8-[(2-hydroxyethyl) [8-(nonyloxy)-8-oxooctyl]amino]-octanoic acid, 1-octylnonyl ester (Lipid 5), 8-[[8-[(1-ethylnonyl)oxy]-8-oxooctyl][3-[[2-(methylamino)-3,4-dioxo-1-cyclobuten-1-yl]amino]propyl]amino]-octanoic acid, 1-octylnonyl ester (Lipid 29), 9,12-octadecadienoic acid, (9Z,12Z)-1,1′-[2-[[[[3-(diethylamino) propoxy]carbonyl]oxy]methyl]-1,3-propanediyl]ester (Ionizable Lipid 4), 1,2-dilinoleyloxo-3-(2-N,N-dimethylamino) ethoxypropane (DLin-EG-DMA), dilinoleyl-methyl-4-dimethylaminobutyrate (DLin-MC3-DMA), 1,1′-[[3-[4-[2-[[3-[bis(2-hydroxyhexadecyl)amino]-2-ethoxypropyl](2-hydroxyhexadecyl)amino]ethyl]-1-piperazinyl]-2-ethoxypropyl]imino]bis-2-hexadecanol (Lipid C₃), 4-methyl-1-piperazinepropanoic acid, 2-[di-(9Z,12Z)-9,12-octadecadien-1-ylamino]ethyl ester (Lipid 10), 7-(((4-(dimethylamino)butanoyl)oxy)((9Z,12Z)-octadeca-9,12-dien-1-yl)amino)heptyl decanoate (Lipid 16), di((9Z,12Z)-octadeca-9,12-dien-1-yl) 4,4′-(2-(tert-butoxycarbonyl)-1-(2-(2-ethylpiperidin-1-yl)ethyl)-2,5-dihydro-1H-imidazole-5,5-diyl)dibutyrate (Lipid 331), N-[3-(dimethylamino)propyl]-4,5,6,7-tetrahydro-6-[(9Z,12Z)-1-oxo-9,12-octadecadien-1-yl]-2-[[(9Z,12Z)-1-oxo-9,12-octadecadien-1-yl]amino]-thieno[2,3-c]pyridine-3-carboxamide (Lipid 29d), 1,1′,1″,1′-(((((3R,3aS,6S,6aS)-hexahydrofuro[3,2-b]furan-3,6-diyl)bis(oxy))bis(propane-3,1-diyl))bis(azanetriyl))tetrakis(dodecan-2-ol) (Lipid DIM1), bis(2-hexyldecyl) 6,6′-((3-(bis(2-hydroxyethyl)amino)propyl)azanediyl)dihexanoate (Lipid U101), 9Z,12Z-octadecadienoic acid, 1,1′,1″,1′″-[(3,6-dioxo-2,5-piperazinediyl)bis(4,1-butanediylnitrilodi-2,1-ethanediyl)]ester (OF-Deg-Lin), 1,1′-((2-(4-(2-((2-(bis(2-hydroxydecyl)amino)ethyl)(2-hydroxydecyl)amino)ethyl)piperazin-1-yl)ethyl)azanediyl)bis(decan-2-ol) (C10-200), 1-[3-(dimethylamino)propyl]-5,5-di-(8Z)-8-heptadecen-1-yl-2,5-dihydro-1H-imidazole-2-carboxylic acid, ethyl ester (A12-Iso5-2DC18), 2-butyl-octanoic acid, 1,1′-[[(3-hydroxypropyl)imino]di-9,1-nonanediyl]ester (Lipid III-45), bis(2-hexyldecyl) 6,6′-((3-(bis(2-hydroxyethyl)amino)propyl)azanediyl)dihexanoate (Lipid U-101), 8-[(2-hydroxyethyl) (2-hydroxytetradecyl)amino]-octanoic acid, 1-octylnonyl ester (Lipid HTO12), 8-[(3-hydroxypropyl) [6-oxo-6-(undecyloxy) hexyl]amino]-octanoic acid, 1-octylnonyl ester (Lipid C-1), tetra((Z)-non-2-en-1-yl) 3,3′,3″,3″-(((methylazanediyl)bis(propane-3,1-diyl))bis(azanetriyl))tetrapropionate (3060i9-cis2), 1-methyl-4-piperidinecarboxylic acid, 11-[(2-hexyl-1-oxodecyl)oxy]-5-[6-[(2-hexyl-1-oxodecyl)oxy]hexyl]-5-hydroxyundecyl ester (CL15F6), N,N′-(piperazine-1,4-diylbis(propane-3,1-diyl))bis(3-(didecylamino) propanamide) (PPZ-A10), 2-((4-(((2-(pyrrolidin-1-yl)ethyl)carbamoyl)oxy)decanoyl)oxy)propane-1,3-diyl(9Z,9′Z,12Z,12′Z)-bis(octadeca-9,12-dienoate) (Lipid 50), 1,2-distearyloxy-N,N-dimethyl-3-aminopropane (DSDMA), N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy) propyl)-N,N,N-trimethylammonium chloride or 1,2-dioleyloxy-3-trimethylaminopropane chloride salt), N-(1-(2,3-dioleyloxy) propyl)-N,N,N-trimethylammonium chloride (DOTMA), N,N-dimethyl-2,3-dioleyloxy) propylamine (DODMA), cKK, 3,6-bis [4-[bis(2-hydroxydodecyl)amino]butyl]-2,5-piperazinedione (cKK-E12), 1,2-diLinoleyloxy-N,N-dimethylaminopropane (DLinDMA), 1,2-dilinolenyloxy-N,N-dimethylaminopropane (DLenDMA), 1,2-di-y-linolenyloxy-N,N-dimethylaminopropane (y-DLenDMA), 98N12-5, 1,2-dilinoleylcarbamoyloxy-3-dimethylaminopropane (DLin-C-DAP), 1,2-dilinoleyoxy-3-(dimethylamino) acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.CI), imidazole cholesterol esters, HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-dilinoleyl-4-dimethylaminoethyl-[1,3]-dioxolane) HGT4003, 1,2-dilinoleoyl-3-trimethylaminopropane chloride salt (DLin-TAP.CI), 1,2-dilinoleyloxy-3-(N-methylpiperazino) propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino) ethoxypropane (DLin-EG-DM A), 2,2-dilinoleyl-4-dimethylaminomethyl-[1,3]-dioxolane (DLin-K-DMA), (3aR,5s,6aS)—N,N-dimethyl-2,2-di((9Z,12Z)-octadeca-9,12-dienyl)tetrahydro-3aH-cyclopenta [d][1,3]dioxol-5-amine, (6Z,9Z,28Z,31Z)-heptatriaconta-6,9,28,31-tetraen-19-yl-4-(dimethylamino) butanoate (MC3), ALNY-100 ((3aR,5s,6aS)—N,N-dimethyl-2,2-di((9Z,12Z)-octadeca-9,12-dienyl)tetrahydro-3aH-cyclopenta [d][1,3]dioxol-5-amine)), 1,1′-(2-(4-(2-((2-(bis(2-hydroxydodecyl)amino) ethyl) (2-hydroxydodecyl)amino) ethyl) piperazin-1-yl)ethylazanediyl)didodecan-2-ol (Cl2-200), 2,2-dilinoleyl-4-(2-dimethylaminoethyl)-[1,3]-dioxolane (DLin-K—C2-DMA), NC98-5 (4,7,13-tris(3-oxo-3-(undecylamino) propyl)-N1,N16-diundecyl-4,7,10,13-tetraazahexadecane-1,16-diamide), (6Z,9Z,28Z,31 Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), N-(1-(2,3dioleyloxy) propyl)-N-(2-(sperminecarboxamido)ethyl)-N,N-dimethylammonium trifluoroacetate (DOSPA), or dioctadecylamidoglycyl carboxyspermine (DOGS), or combinations thereof.

### Containers and Mixing

One or more RNA stabilizing substance and one or more RNA substance may be contained in a chamber, such as the non-limiting example schematically illustrated in FIG. 65. Chambers may include containers, such as syringes or vials, which may hold a combination of at least one or more RNA stabilizing substance and at least one or more RNA substance and the containers may also contain one or more additional substance, such as at least one or more cellular uptake agents, or additive substances, or one or more additional RNA stabilizing substances as non-limiting examples.

Furthermore, chambers may include containers, such as syringes or vials, which may hold a combination of at least one or more RNA stabilizing substance and at least one or more RNA substance and the containers may also contain one or more additional substance, such as at least one or more cellular uptake agents or other substances, that are kept separate from the RNA stabilizing substance and the RNA substance until close to the time of use.

In some embodiments, chambers, including vials or syringes (including single compartment or multicompartment syringes) as non-limiting examples, may contain one or more RNA stabilizing composition described herein. As a non-limiting example, chambers may contain one or more RNA stabilizing composition described herein, wherein one or more components of a composition may be kept separate from the RNA stabilizing substance and the RNA substance (such as in different compartments of a single chamber, such as a multi-compartment syringe, or in separate chambers, as non-limiting examples) until close to the time of use.

As a non-limiting example, a container may hold a combination of materials comprising at least one or more RNA substance and at least one or more RNA stabilizing substance that is ready for injection after removal from the container such as by withdrawal using a syringe and needle. As a non-limiting example, a container, which may be a vial as a non-limiting example, may hold a combination of materials that is a concentrate for injection comprising at least one or more RNA substance and at least one or more RNA stabilizing substance that after dilution is ready for injection upon removal from the container such as by withdrawal using a syringe and needle. As a non-limiting example, the dilution may occur by adding a solution comprising water to a container, which may be a vial, that is partially filled to leave volume for adding and mixing diluting solution. As a non-limiting example, the diluting solution may be 0.9% sodium chloride (normal saline, preservative-free). As a non-liming example, the container may be a multi-dose container, which may be a multi-dose vial, containing at least two doses or containing at least 5 doses or containing at least 10 doses or containing at least 15 doses. As a non-liming example, the container may be a single dose or a multi-dose vial that is at least 0.1 mL size, or at least 0.2 mL size, or at least 0.5 mL size, or at least 1 mL size, or at least 2 ml size, or at least 5 ml size, or at least 10 ml size, or at least 20 ml size.

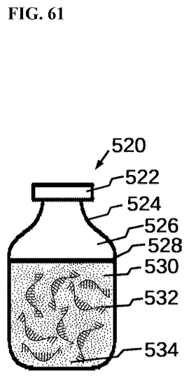

As a non-limiting example, a vial 520, as shown in FIG. 61, may have a seal 522 affixed to a container 524 forming an enclosed volume 526 that is at least partially filled to a predetermined quantity or level 528 with liquid material 534 comprising at least one RNA stabilizing substance 530 (depicted as dots in FIG. 61) at least one RNA substance 532 (depicted as the wave lines and straight lines in FIG. 61). As a non-limiting example, seal 522 may comprise an elastomeric reseal that may be penetrated by a needle (not shown) affixed to syringe (not shown), enabling liquid material 534 to be at least partially withdrawn from vial 520 and transferred to the syringe using techniques known to those skilled in the art. A non-limiting alternative example use of vial 520 is to have the syringe at least partially contain a material, such as a diluent comprising water as a non-limiting example, suitable for diluting liquid material 534 comprising at least RNA stabilizing substance 530 and RNA substance 532 by injecting the material from the syringe (not shown) into vial 520 prior to withdrawing at least part of the combined, and as a non-limiting example, mixed, materials from vial 520 into the syringe. One or more syringes may be used to withdraw materials from vial 520. Other devices besides syringes may be used to withdraw materials, non-limiting examples include pumping devices and devices that pressurize enclosed volume 526 of vial 520.

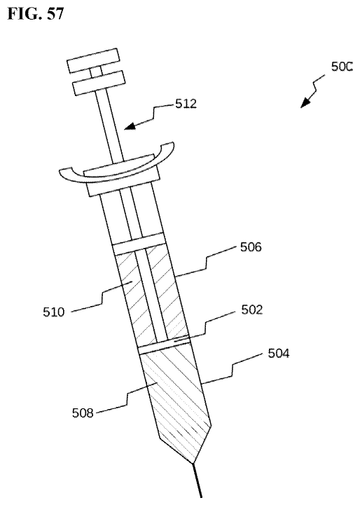

As a non-limiting example, a multi-compartment syringe 500, as shown in FIG. 57, with a breakable seal 502 between compartments may have a first mixture 508 including at least one or more RNA stabilizing substance, at least one or more RNA substance, and at least one or more cellular uptake agents, such as lipids, in one compartment 504 and an aqueous solution 510, such as a diluent comprising water, in a second compartment 506. At time of use the seal 502 between the compartments 504 and 506 is broken and the contents of both compartments 504 and 506 are mixed to reduce the viscosity of one component or to induce at least one or more cellular uptake agent, such as lipids, to at least partially complex with or at least partially encapsulate one or more RNA substance prior to use, as a non-limiting example. For example, the seal 502 may be broken by advancing or turning a plunger assembly 512 or portion thereof to puncture a membrane or otherwise enable mixing. In another non-limiting example, at least one or more RNA substance and at least one or more RNA stabilizing substance may be in one compartment and at least one or more cellular uptake agent, such as a lipid or polymer, may be in a second compartment with a breakable seal between the compartments. At time of use the seal is broken and the contents of the two compartments are mixed to induce at least one or more cellular uptake agent, such as a lipid or polymer, to combine with one or more RNA substance prior to use.

As another non-limiting example, syringe 500 shown in FIG. 57 may have a first composition or mixture 508 comprising one or more RNA substance and one or more RNA stabilizing substance or one or more cellular uptake agent in the first compartment 504 and the second compartment 506 may have a diluent comprising water (such as a buffer comprising tris, phosphate, or other suitable buffering agent) 510 that upon breaking seal 502 at least partially mixes or combines with the composition in compartment 504 prior to injection. As a non-limiting example, a diluent in the second compartment may comprise a buffering agent that may adjust the pH of the composition in the first compartment, such as adjusting the pH to about 5-9, or to about 6-8, or to about 7-8.

As a non-limiting example one or more components of a composition may be stored in separate compartments of a multi-compartment syringe, wherein the components may be mixed, such as by breaking a seal separating the two components of the composition prior to use. As a non-limiting example, one compartment may contain an RNA stabilizing composition with a selectively adjustable viscosity and a second compartment may contain a diluent (such as a buffer or water) that when mixed with the RNA stabilizing composition may reduce the viscosity to a specified value or range prior to use.

As a non-limiting example, a diluent in the second compartment may comprise a buffering agent that may adjust the pH of the composition in the first compartment, such as adjusting the pH to about 6 or greater, or to about 6.5 or greater, or to about 7 or greater. As a non-limiting example, a diluent in the second compartment may dilute the composition in the first compartment by at least 10%, or by at least 20%, or by at least 30%, or by at least 40%, or by at least 50%, or by at least 80%, or by at least 100%, or by at least 150%, or by at least 200%, or by at least 300%. As a non-limiting example, diluting by 100% means doubling the original volume, such as adding 5 mL to an original volume of 5 mL resulting in a final volume of 10 mL leading to a diluted concentration that is one-half of the beginning (undiluted) concentration.

As another non-limiting example, syringe 500 shown in FIG. 57 may have a first composition or mixture 508 comprising one or more RNA substance and one or more RNA stabilizing substance or one or more cellular uptake agent (such as one or more ionizable lipid, as a non-limiting example) in the first compartment 504 and the second compartment 506 may have one or more additional cellular uptake agents (such as a PEG-lipid, neutral-lipid, or cholesterol as non-limiting examples) 510 that upon breaking seal 502 at least partially mixes or combines with the composition in compartment 504 prior to use, such as by injection. As a non-limiting example, one or more additional cellular uptake agent in the second compartment may promote encapsulation or at least partially encapsulate or at least partially complex with one or more RNA substance in the first compartment. As a non-limiting example, one or more compartment of a multi-compartment syringe as shown in FIG. 57 may contain one or more composition or substances described herein, including one or more RNA stabilizing composition, or one or more RNA stabilizing substance, RNA substance, cellular uptake agent, additive substance, buffering agent, or water, or combinations thereof, wherein a compartment may contain individual substances or combinations of one or more substances.

Embodiments of one or more multicompartment syringe may comprise one or more RNA stabilizing composition described herein, comprising one or more RNA stabilizing substance and one or more RNA substance or one or more additional substance, such as one or more cellular uptake agent, additive substance, inorganic cation (or salts thereof), buffer, or water as non-limiting examples. As a non-limiting example one or more substance (e.g. an RNA stabilizing substance or RNA substance) or composition (e.g. an RNA stabilizing composition) described herein may be stored separately in separate compartments and then mixed (such as upon breaking seal 502 as a non-limiting example) prior to an intended use (such as administering a pharmaceutical composition to a subject in need thereof as a non-limiting example).

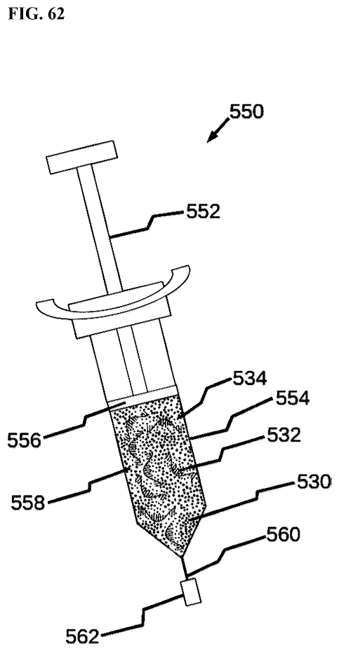

As a non-limiting example, single compartment syringe 550, is shown in FIG. 62, As a non-limiting example, a syringe 550, may have plunger assembly 552 slidably moving relative to syringe container 554 with sliding seal element 556 as part of plunger assembly 552 forming an enclosed volume 558 that is at least partially filled with liquid material 534 comprising at one least RNA stabilizing substance 530 (depicted as dots in FIG. 62) and at least one RNA substance 532 (depicted as the wave lines and straight lines in FIG. 62). Delivery port 560 fluidically communicates with enclosed volume 558 and may, as non-limiting examples, be a needle or an opening (not shown) syringe 550 to which a needle or other delivery component attaches. Port sealing element 562 substantially retains liquid material 534 inside enclosed container space 558 after liquid material 534 is loaded into enclosed container space such as during prefilling syringe 550 during manufacturing or other preparation. Port sealing element 562 may, as non-limiting examples, seal port 560 by surrounding the outside of the port or by plugging the inside of one or more fluid channels in port 560. Liquid material 534 is transferred from the syringe using techniques known to those skilled in the art by operating plunger assembly 552.

In other embodiments of the present disclosure, chambers or containers may be embedded complexes comprising at least one or more RNA stabilizing substance and at least one or more RNA substance that remain substantially non-communicative across at least one boundary until interaction with a biologic material, as a non-limiting example the biologic material may be fluid or tissue in a living organism, alters the embedded complex so that at least one RNA substance moves from the embedded complex into an organism, such as an organ or tissue in an animal or human. As a non-limiting example, the embedded complex may be implanted in a living organism and upon being altered by biologic material of the living organism at least one RNA substance at least partially moves from the embedded complex into at least one tissue of the living organism. As a non-limiting example, one or more other substances besides an RNA substance may also at least partially move from the embedded complex into at least one tissue. As non-limiting examples, the substances besides RNA substances may comprise at least one of RNA stabilizing substances or cellular uptake agents; or additional materials antagonistic to cellular activity either alone or in combination with cellular processes influenced by one or more RNA substance, and substances benefiting cellular activity either alone or in combination with cellular processes influenced by one or more RNA substance. As a non-limiting example, the embedded complex may deliver one or more RNA substances that produce a response in neoplasms that alter, inhibit, or terminate metabolic activity of at least one neoplastic cell. As a non-limiting example, the embedded complex may be implanted in at least the vicinity or at least partially in a neoplasm or where communication between at least one RNA substance and at least one neoplasm may occur through biologic transport processes such through fluid transfer, which may include transfer through the blood system or through the lymph system. As a non-limiting example, the embedded complex may comprise at least one RNA substance that affects the survival or growth of at least one non-malignant neoplasm. As a non-limiting example, the embedded complex may comprise at least one RNA substance that affects the survival or growth of at least one malignant neoplasm.

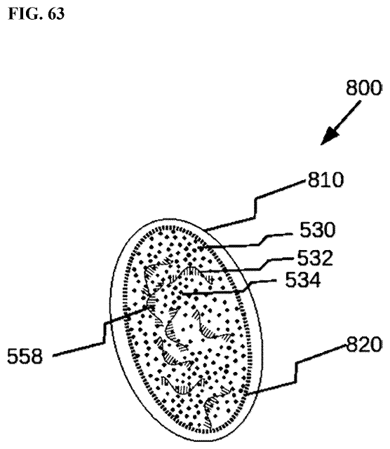

As a non-limiting example, embedded complex 800, is shown in FIG. 63. As a non-limiting example, embedded complex 800, may have a packaging layer 810 that protects the implantable material during shipping or storage, and provides a sterile barrier. Polymeric material 820 forms a volume (such as gel or network or an at least partially enclosed space) 558 that at least partially contains internal material 534 comprising at least one RNA substance 532 (depicted as the wave lines and straight lines in FIG. 63). Polymeric material 820 may comprise an RNA stabilizing substance, wherein the RNA substance may be embedded, bonded to (e.g. via hydrogen bonding or electrostatic interactions as non-limiting examples), or incorporated into pores of the polymeric material as a non-limiting example. As another non-limiting example, polymeric material 820 may comprise one or more RNA stabilizing polymer substance (such as a modified carbohydrate substance or modified polysaccharide substance as a non-limiting example). Polymeric material 820 may degrade when exposed to biologic materials, such biologic fluids and such degradation may allow internal material 534 to enter one or more tissues in which embedded complex 800 is embedded, such as by being implanted in one or more tissues. The tissues in which embedded complex 800 is embedded may be from implanting into animal tissue and as non-limiting examples, the animal tissues may be one or more mammalian tissues. As a non-limiting example, the mammalian tissues may be one or more human tissues.

In some embodiments polymeric material 820 may be substituted with one or more RNA stabilizing composition described herein comprising a gel (such as a hydrogel as a non-limiting example) or viscous fluid (such as a thixotropic fluid as a non-limiting example) comprising one or more monomeric RNA stabilizing substance, wherein the composition comprising a gel or viscous fluid may also comprise water. As a non-limiting example, a network of monomers comprising one or more RNA-stabilizing substance (such as a mono-nucleoside substance or mono-nucleotide substance as a non-limiting example) may comprise a supramolecular assembly of individual monomers into structures (such as helices, tubes, rods, sheets, rings, crystals, or fibrils, as non-limiting examples) assembled via hydrogen bonds, base stacking, or electrostatic interactions as described herein. As a non-limiting example, mono-nucleoside substances or mono-nucleotide substances (such as guanosine-5′-monophosphate, as a non-limiting example) may be used to produce compositions comprising a network of monomers with gel-like properties (such as allowing the diffusion of water or ions through a viscous fluid or ionically bonded network, as a non-limiting example), wherein the network of monomers or viscosity of the composition may be adjusted by changing the pH, or changing the ionic strength, or increasing or decreasing the concentration of one or more constituents within a composition as described herein. As a non-limiting example, these networks of monomers comprising one or more mono-nucleoside substance or one or more mono-nucleotide substance may be fully or partially reversible or may be reassembled in selected ways to produce tunable (e.g. reversible, semi-reversible, or selectively adjustable, as non-limiting examples) compositions or RNA storage environments comprising one or more RNA substance and one or more RNA stabilizing substance.

As a non-limiting example, embedded complex 800 comprising material 820, wherein material 820 may be a polymeric material or network of monomers comprising one or more RNA stabilizing substance (such as one or more mono-nucleoside substance or one or more mono-nucleotide substance), may disintegrate or change physical properties (such as change from solid to liquid or reduce viscosity as non-limiting examples) in response to a change in pH or upon contact with one or more biologic materials (e.g. one or more biological tissues or fluids as non-limiting examples). Embodiments of one or more embedded complex may comprise one or more RNA stabilizing composition described herein, comprising one or more RNA stabilizing substance and one or more RNA substance or one or more additional substance such as one or more cellular uptake agent, additive substance, inorganic cation (including salts thereof), buffer, or water as non-limiting examples.

Embedded complex 800 may have a diffusion controlling barrier in addition to or instead of polymeric material or network of monomers that degrades when exposed to biologic materials.

As another non-limiting example, embedded complex 800 may be a container such as a tablet, lozenge, capsule, gel capsule, or other chamber that contacts biologic fluids without being implanted. As non-limiting examples, embedded complex 800 may be ingested orally, be a suppository, or be at least part of an inhalable material such as particles or a mist or droplets produced by an inhaler or nebulizer.

As non-limiting examples, chambers or containers may hold one or more RNA stabilizing composition described herein and may be made of any materials suitable for storing and shipping at least one or more RNA stabilizing substance and at least one or more RNA substance including, but not limited to, glass, metal, ceramic, plastic or other polymeric material that does not degrade or modify the chamber's or container's contents or be degraded or modified by the chamber's or container's contents. Chambers or containers may have an access port that is penetrated or removed to access the interior of the chamber or container including accessing at least part of the contents of the chamber or container. Chambers or containers may have an access port, such as a screw lid, removable tab, combination plug or cap or closure that may be penetrated, such as by a hollow tube, such as a hollow needle, to access the interior of the chamber or container either to add one or more materials to the contents of the chamber or container, or to remove at least part of the contents from the interior of the chamber or container.

Chambers or containers may also be used to add materials to the at least one or more RNA stabilizing composition described herein. As a non-limiting example, a syringe containing a substance comprising at least one or more lipid may have at least part of its contents transferred to the interior of a chamber or container through the access port such as by using a hollow needle penetrating a resealing closure. As another non-limiting example, the syringe may contain a diluent comprising water that is at least partially transferred to a chamber or container having contents comprising at least one RNA stabilizing substance and at least one RNA substance.

The chambers or containers, described above, may also hold a combination comprising at least one or more RNA stabilizing substance and at least one or more RNA substance, or at least one or more cellular uptake agent, or one or more additional substances described herein. The chambers or containers, described above, may be made of any materials suitable for storing and shipping at least one RNA stabilizing substance, at least one RNA substance or at least one cellular uptake agent, or one or more additional substances including, but not limited to, glass, metal, ceramic, plastic or other polymeric material that does not degrade or modify the chamber's or container's contents or be degraded or modified by the chamber's or container's contents.

The chambers or containers, described above, may be used to add materials to one or more RNA stabilizing composition described herein such as one or more composition comprising at least one or more RNA stabilizing substance, at least one or more RNA substance, and at least one or more cellular uptake agent, as a non-limiting example. As another non-limiting example, the syringe may contain a diluent comprising water that is at least partially transferred to a chamber or container having contents comprising at least one RNA stabilizing substance and at least one RNA substance and at least one cellular uptake agent, as a non-limiting example.

### Uses and Applications: Environment and Storage

In other methods of use one or more RNA stabilizing composition described herein comprising at least one or more RNA stabilizing substance and at least one or more RNA substance may be stored in a chamber at a defined minimum temperature for a defined duration of time, wherein the defined minimum temperature may be at least about −20° C. or greater for a defined duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months, or 24 months and the maximum time is greater than the minimum time and may be at least one of 6 hours, 24 hours, 48 hours, 72 hours, 100 hours, 7 days, 14 days, 30 days, 60 days, 3 months, 6 months, 12 months, 18 months, 24 months, 48 months, 5 years, 10 years, or 20 years. As non-limiting examples the exposure to temperatures of at least the defined minimum temperature may be continuous or the exposure may be intermittent.

In other methods of use one or more RNA stabilizing composition described herein comprising at least one or more RNA stabilizing substance and at least one or more RNA substance may be stored in a chamber at a defined minimum temperature for a defined duration of time, wherein the defined minimum temperature may be at least about 0° C. or greater for a defined duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months, or 24 months and the maximum time is greater than the minimum time and may be at least one of 6 hours, 24 hours, 48 hours, 72 hours, 100 hours, 7 days, 14 days, 30 days, 60 days, 3 months, 6 months, 12 months, 18 months, 24 months, 48 months, 5 years, 10 years, or 20 years. As non-limiting examples the exposure to temperatures of at least the defined minimum temperature may be continuous or the exposure may be intermittent.

In other methods of use one or more RNA stabilizing composition described herein comprising at least one or more RNA stabilizing substance and at least one or more RNA substance may be stored in a chamber at a defined minimum temperature for a defined duration of time, wherein the defined minimum temperature may be at least about 4° C. or greater for a defined duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months, or 24 months and the maximum time is greater than the minimum time and may be at least one of 6 hours, 24 hours, 48 hours, 72 hours, 100 hours, 7 days, 14 days, 30 days, 60 days, 3 months, 6 months, 12 months, 18 months, 24 months, 48 months, 5 years, 10 years, or 20 years. As non-limiting examples the exposure to temperatures of at least the defined minimum temperature may be continuous or the exposure may be intermittent.

In other methods of use one or more RNA stabilizing composition described herein comprising at least one or more RNA stabilizing substance and at least one or more RNA substance may be stored in a chamber at a defined minimum temperature for a defined duration of time, wherein the defined minimum temperature may be at least about 10° C. or greater for a defined duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months, or 24 months and the maximum time is greater than the minimum time and may be at least one of 6 hours, 24 hours, 48 hours, 72 hours, 100 hours, 7 days, 14 days, 30 days, 60 days, 3 months, 6 months, 12 months, 18 months, 24 months, 48 months, 5 years, 10 years, or 20 years. As non-limiting examples the exposure to temperatures of at least the defined minimum temperature may be continuous or the exposure may be intermittent.

In other methods of use one or more RNA stabilizing composition described herein comprising at least one or more RNA stabilizing substance and at least one or more RNA substance may be stored in a chamber at a defined minimum temperature for a defined duration of time, wherein the defined minimum temperature may be at least about 20° C. or greater for a defined duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months, or 24 months and the maximum time is greater than the minimum time and may be at least one of 6 hours, 24 hours, 48 hours, 72 hours, 100 hours, 7 days, 14 days, 30 days, 60 days, 3 months, 6 months, 12 months, 18 months, 24 months, 48 months, 5 years, 10 years, or 20 years. As non-limiting examples the exposure to temperatures of at least the defined minimum temperature may be continuous or the exposure may be intermittent.

In other methods of use one or more RNA stabilizing composition described herein comprising at least one or more RNA stabilizing substance and at least one or more RNA substance may be stored in a chamber at a defined minimum temperature for a defined duration of time, wherein the defined minimum temperature may be at least about 30° C. or greater for a defined duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months, or 24 months and the maximum time is greater than the minimum time and may be at least one of 6 hours, 24 hours, 48 hours, 72 hours, 100 hours, 7 days, 14 days, 30 days, 60 days, 3 months, 6 months, 12 months, 18 months, 24 months, 48 months, 5 years, 10 years, or 20 years. As non-limiting examples the exposure to temperatures of at least the defined minimum temperature may be continuous or the exposure may be intermittent.

In other methods of use one or more RNA stabilizing composition described herein comprising at least one or more RNA stabilizing substance and at least one or more RNA substance may be stored in a chamber at a defined minimum temperature for a defined duration of time, wherein the defined minimum temperature may be at least about 40° C. or greater for a defined duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months, or 24 months and the maximum time is greater than the minimum time and may be at least one of 6 hours, 24 hours, 48 hours, 72 hours, 100 hours, 7 days, 14 days, 30 days, 60 days, 3 months, 6 months, 12 months, 18 months, 24 months, 48 months, 5 years, 10 years, or 20 years. As non-limiting examples the exposure to temperatures of at least the defined minimum temperature may be continuous or the exposure may be intermittent.

In other methods of use one or more RNA stabilizing composition described herein comprising at least one or more RNA stabilizing substance and at least one or more RNA substance may be stored in a chamber at a defined minimum temperature for a defined duration of time, wherein the defined minimum temperature may be at least about 50° C. or greater for a defined duration between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months, or 24 months and the maximum time is greater than the minimum time and may be at least one of 6 hours, 24 hours, 48 hours, 72 hours, 100 hours, 7 days, 14 days, 30 days, 60 days, 3 months, 6 months, 12 months, 18 months, 24 months, 48 months, 5 years, 10 years, or 20 years. As non-limiting examples the exposure to temperatures of at least the defined minimum temperature may be continuous or the exposure may be intermittent.

In other methods of use one or more RNA stabilizing composition described herein comprising at least one or more RNA stabilizing substance and at least one or more RNA substance may be stored in a chamber at a defined minimum temperature, for a defined minimum period of time, wherein the defined minimum temperature may be at least −20° C., −10° C., 0° C., 4° C., 10° C., 15° C., 20° C., 30° C., 40° C., or 50° C., and the defined minimum period of time may be at least one of 1 hour, 2 hours, 4 hours, 8 hours, 12 hours, 18 hours, 24 hours, 48 hours, 72 hours, 100 hours, 7 days, 14 days, 30 days, 60 days, 3 months, 6 months, 12 months, 18 months, or 24 months. As non-limiting examples the exposure to temperatures of at least the defined minimum temperature may be continuous or the exposure may be intermittent. As another non-limiting example, one or more RNA stabilizing composition (such as a pharmaceutical composition as a non-limiting example) may be stored in a chamber at a defined minimum temperature (such as at least 15° C.) for a defined period of time (such as at least 30 days), wherein the exposure to temperatures of at least the defined minimum temperature may be continuous or the exposure may be intermittent.

Embodiments of the foregoing methods of use include using one or more RNA stabilizing substance mixed with one or more RNA substance. Some embodiments of the foregoing methods of use may comprise one or more RNA stabilizing composition described herein (such as a pharmaceutical composition as a non-limiting example). Some embodiments of the foregoing methods of use may comprise one or more RNA stabilizing composition described herein and may also comprise one or more additional substance such as one or more cellular uptake agent, additive substance, inorganic cation (or salts thereof), buffer, or water as non-limiting examples.

In some methods of use one or more RNA stabilizing composition described herein comprising at least one or more RNA stabilizing substance and at least one or more RNA substance may be a composition or combination stored at a temperature less than the melting point of the combination of substances.

In some methods of use one or more RNA stabilizing composition described herein comprising at least one or more RNA stabilizing substance and at least one or more RNA substance and at least one or more cellular uptake agent may be a composition or combination stored at a temperature less than the melting point of the combination of substances.

In some methods of use one or more RNA stabilizing composition described herein, may be a composition wherein the composition may be a solid when stored at a temperature of about 4° C., or about 10° C., or about 20° C., or about 30° C. As a non-limiting example, a composition comprising one or more RNA stabilizing substance and one or more RNA substance may be a solid when stored at about 20° C.

In some methods of use one or more RNA stabilizing composition described herein, may be a composition wherein the composition may be a gel (e.g. hydrogel) or viscous fluid comprising water (e.g. thixotropic fluid comprising water) when stored at a temperature of about 4° C., or about 10° C., or about 20° C., or about 30° C. As a non-limiting example, a composition comprising one or more RNA stabilizing substance and one or more RNA substance and water may be a gel (e.g. hydrogel) when stored at about 20° C.

### Applications and Methods of Use

Some embodiments of the present disclosure are the methods whereby one or more RNA stabilizing substance may be combined, such as by mixing, with at least one or more RNA substance to produce a composition or mixture comprising at least one or more RNA stabilizing substance and at least one or more RNA substance. As a non-limiting example one or more RNA substance may be mixed (or otherwise combined) with one or more RNA stabilizing substance, to produce one or more RNA stabilizing composition described herein.

Non-limiting example methods for combining one or more RNA stabilizing substance and one or more RNA substance may include, mixing, pipetting, blending, stirring, diffusion, inverting, submerging, vortexing, or shaking such that at least one or more RNA stabilizing substance is at least intimately associated with or at least partially contacting or at least partially encapsulating at least one or more RNA substance.

Some embodiments of the present disclosure are the methods whereby one or more RNA stabilizing substance may be combined with one or more RNA substance by mixing the substances using known methods. These methods may include, but are not limited to, stirring, fluid flow agitation, vortexing, inverting, pipetting, blending, multiple channel fluidics, low shear blending, microfluidic mixing, diffusion, or using static mixers. In other embodiments, one or more of these methods may be used to combine one or more other substances, including, but not limited to cellular uptake agents, water, additive substances, inorganic cations (or salts thereof), or additional RNA stabilizing substances, with one or more RNA substance and one or more RNA stabilizing substance.

Embodiments of methods described herein may be independent of the order in which each substance may be combined or mixed together. As a non-limiting example one or more RNA stabilizing substance may be combined with one or more RNA substance or one or more RNA substance may be combined with one or more RNA stabilizing substance by the same method.

Some embodiments of the present disclosure are the methods whereby one or more RNA stabilizing substances may be combined with at least one or more RNA substance in a chamber or container. As a non-limiting example, a chamber or container may comprise glass, plastic, ceramic, an elastomer, a polymer, metal, or other suitable material described herein.

Some embodiments of the present disclosure are the methods whereby one or more RNA stabilizing substances may be combined with at least one or more RNA substance and placed in or introduced into a chamber or container. As a non-limiting example, a chamber or container may comprise glass, plastic, ceramic, an elastomer, a polymer, metal, or other suitable material described herein.

In some embodiments, single doses of an RNA stabilizing composition may be packaged and sealed (such as a hermetically sealed chamber or container, such as a vial or prefilled syringe, or multicompartment syringe as non-limiting examples). In some embodiments, multiple doses of an RNA stabilizing composition may be packaged and sealed in one packaging unit (such as a hermetically sealed chamber or container, such as a vial or prefilled syringe, or multicompartment syringe as non-limiting examples). As a non-limiting example, single doses or multiple doses of an RNA stabilizing composition may be packaged in chambers. As a non-limiting example, single doses or multiple doses of an RNA stabilizing composition may be packaged in chambers and hermetically sealed.

In some embodiments of the present disclosure one or more RNA stabilizing substance may also be used in conjunction with lyophilization of at least one or more RNA substance, wherein one or more RNA stabilizing composition may be lyophilized, such that an RNA stabilizing composition may comprise a defined maximum water content (such as less than 10% water weight percent, as a non-limiting example). As a non-limiting example, an RNA stabilizing composition may be packaged or shipped wherein an RNA stabilizing composition has been lyophilized comprising a defined maximum water content. As a non-limiting example, an RNA stabilizing composition that has been lyophilized may comprise a defined maximum water content (such as less than 10% water weight percent, as a non-limiting example). As a non-limiting example, an RNA stabilizing composition that has been lyophilized may comprise a maximum water weight percent of less than 50%, or less than 40%, or less than 30%, or less than 20%, or less than 15%, or less than 10%, as non-limiting examples. As a non-limiting example, lyophilization may include freeze drying, evaporation, sublimation, evaporation or sublimation under vacuum, evaporation or sublimation under standard atmospheric conditions (e.g. without vacuum), or combinations thereof.

In some embodiments of the present disclosure one or more RNA stabilizing composition described herein may be substantially free of lyophilization, such that an RNA stabilizing composition may comprise a defined minimum water content (such as at least 10% water weight percent or greater, as a non-limiting example). As a non-limiting example, an RNA stabilizing composition may be packaged or shipped wherein the RNA stabilizing composition has not been lyophilized (e.g. is substantially free of lyophilization, as a non-limiting example). As a non-limiting example, an RNA stabilizing composition substantially free of lyophilization may comprise a defined minimum water content (such as at least 10% water weight percent or greater, as a non-limiting example). As a non-limiting example, an RNA stabilizing composition substantially free of lyophilization may comprise a minimum water weight percent of at least about 10% or greater, or at least about 15% or greater, or at least about 20% or greater, or at least about 25% or greater, or at least about 30% or greater, or at least about 40% or greater, or at least about 50% or greater, or at least about 60% or greater, as non-limiting examples.

### Producing Compositions

RNA stabilizing compositions described herein may be produced comprising at least one RNA substance and at least one RNA stabilizing substance. Additionally, RNA stabilizing compositions may be used to produce one or more products or compositions comprising at least one RNA substance. As non-limiting examples, at least one RNA stabilizing composition of the present disclosure may be produced for later use. As a non-limiting example, an RNA stabilizing composition may be used to produce a pharmaceutical composition comprising at least one RNA substance for later use.

Non-limiting example methods for producing an RNA stabilizing composition may comprise one or more method steps described herein. Non-limiting example steps in a method may include producing or assembling an RNA stabilizing composition comprising at least one RNA substance for later use.

Non-limiting example methods for producing an RNA stabilizing composition may comprise combining, such as by mixing, one or more RNA stabilizing substance with at least one or more RNA substance to produce an RNA stabilizing composition. A method may further comprise adding or mixing one or more additional substances described herein to a composition, such as one or more cellular uptake agent, to produce an RNA stabilizing composition further comprising a cellular uptake agent or other substances. A method may also comprise, placing an RNA stabilizing composition into a chamber, wherein, as non-limiting examples, a method may comprise combining one or more components of an RNA stabilizing composition either prior to or after being placed into a chamber. As a non-limiting example, one or more components of an RNA stabilizing composition may be mixed and then placed into a chamber or alternatively, one or more components may be combined inside of a chamber. RNA stabilizing compositions may be contained in one or more chamber described herein, non-limiting examples include vials, syringes, tubes, or multi-compartment syringes. A chamber may also include one or more label, where a label may be affixed to or printed onto the chamber as non-limiting examples. A label on a chamber may comprise information about the contents of the chamber or other suitable information as described herein. Once an RNA stabilizing composition is contained in a chamber, a method may comprise hermetically sealing said chamber. As non-limiting examples, a method may comprise hermetically sealing a chamber containing an RNA stabilizing composition and then at least one of packaging or storing said chamber. A method comprising packaging an RNA stabilizing composition, may comprise packaging the chamber into a walled container, such as a box, bag, or other suitable container described herein. A method comprising packaging an RNA stabilizing composition, may also comprise placing a label on the package with information about the contents of the package or placing instructions for use for one more composition contained within the package. A method may also comprise sealing a package, such as with an adhesive seal or mechanical interference, such as a latch or tab, or other suitable method described herein. Following packaging, a method may comprise shipping or storing a package containing at least one RNA stabilizing composition, wherein a package may be transported or shipped to a desired location, such as a location of use, or stored at a location of use. As a non-limiting example, an intended use of an RNA stabilizing composition comprising at least one RNA substance may be administering a vaccine or therapeutic agent to a subject if the composition is a pharmaceutical composition as a non-limiting example.

A non-limiting example method for producing and using a pharmaceutical composition may comprise, combining, such as by mixing, one or more RNA stabilizing substance with at least one or more pharmaceutically active RNA substance. A method may further comprise adding or mixing one or more additional substances, such as one or more cellular uptake agent, to produce a pharmaceutical composition comprising at least one RNA stabilizing substance, at least one pharmaceutically active RNA substance, and at least one cellular uptake agent. As described above, the order of mixing may be independent of the method steps described. As a non-limiting example an RNA substance may be mixed with a cellular uptake agent and an RNA stabilizing substance or alternatively, an RNA stabilizing substance may be mixed with an RNA substance and a cellular uptake agent using the same method. A method may also comprise, placing the pharmaceutical composition in a chamber (such as a vial or syringe as non-limiting examples) and hermetically sealing said chamber. The chamber may then be packaged as described above and the package may be stored at a location of use or shipped or transported to a location of use as described above. An intended use of the pharmaceutical composition may be administering a medicament, vaccine, or therapeutic agent to a subject in need thereof, wherein the pharmaceutical composition may be administered using a syringe, tablet, patch, inhaler, spray, drops, or other suitable method described herein, as non-limiting examples.

As a non-limiting example, a method for producing an RNA stabilizing composition may be used to produce one or more RNA stabilizing composition described herein, including a pharmaceutical composition, biostimulant composition, or an implant as non-limiting examples.

Additionally, a method for producing an RNA stabilizing composition may also comprise testing for endotoxin, such as using an LAL assay or other method known in the art. As a non-limiting example, an RNA stabilizing composition may be produced with an endotoxin level below a specified value as described herein, such as less than 100 endotoxin units/mL as a non-limiting example.

Additionally, a method may also comprise sterilizing one or more component or composition prior to or after packaging. Non-limiting examples may include, filter sterilizing, autoclave, steam, heat, radiation, UV, or other suitable method described herein.

Other non-limiting example methods or method steps may also comprise, producing an RNA stabilizing composition comprising one or more cellular uptake agent by combining or mixing a defined ratio of RNA substance to cellular uptake agent as measured on a weight-by-weight basis, as described herein. As a non-limiting example, a method for producing an RNA stabilizing composition may also comprise combining or mixing one or more cellular uptake agent with one or more RNA substance at a defined ratio as measured on a weight-by-weight basis. As a non-limiting example, a method for producing an RNA stabilizing composition may also comprise mixing a defined total weight of an RNA substance with a defined total weight of at least one cellular uptake agent, to produce a composition with a defined minimum ratio of RNA to cellular uptake agent as measured on a weight-by-weight basis; such as producing a composition with a ratio of RNA substance to cellular uptake agent of at least 1:2, as a non-limiting example.

Non-limiting embodiments of the present disclosure further provides one or more methods for producing one or more RNA stabilizing composition described herein, wherein a composition may comprise at least one or more RNA substance and one or more RNA stabilizing substance. In some embodiments, non-limiting example methods for producing an RNA stabilizing composition may comprise combining, such as by mixing, one or more RNA stabilizing substance with at least one or more RNA substance to produce an RNA stabilizing composition comprising at least one or more RNA substance and at least one or more RNA stabilizing substance. As a non-limiting example, one or more methods for producing an RNA stabilizing composition may comprise combining one or more RNA substance with one or more RNA stabilizing substance or multiple RNA stabilizing substances to produce an RNA stabilizing composition comprising one or more RNA substance and one or more RNA stabilizing substance or multiple RNA stabilizing substances. Other non-limiting embodiments of methods for producing at least one RNA stabilizing composition may comprise combining one or more additional substances described herein with one or more RNA substance and one or more RNA stabilizing substance to produce an RNA stabilizing composition comprising one or more RNA substance, one or more RNA stabilizing substance, and one or more additional substances. Non-limiting example additional substances may include one or more cellular uptake agents, additional RNA stabilizing substances, additional RNA substances, additive substances, inorganic cations (or salts thereof), buffering agents, solvents, or water.

Non-limiting embodiments of one or more method for producing one or more RNA stabilizing composition described herein, may comprise producing at least one of a pharmaceutical composition, biostimulant composition, or implant. Other non-limiting embodiments of one or more methods for producing one or more RNA stabilizing composition may comprise producing a composition for at least one of delivering or administering at least one RNA substance to at least one of a cell, organ, tissue, plant, insect, animal, mammal, human, or subject in need thereof. As a non-limiting example, a method for producing an RNA stabilizing composition may comprise combining at least one or more RNA substance and at least one or more RNA stabilizing substance, or one or more additional substance (such as one or more cellular uptake agent as a non-limiting example), to produce a composition for at least one of delivering or administering at least one RNA substance to at least one of a cell, organ, tissue, plant, insect, animal, mammal, human, or subject in need thereof. As a non-limiting example, a method for producing a pharmaceutical composition may comprise combining at least one or more pharmaceutically active RNA substance and at least one or more RNA stabilizing substance, or one or more additional substance (such as one or more cellular uptake agent as a non-limiting example), to produce a composition for administering at least one pharmaceutically active RNA substance to a subject in need thereof.

In other embodiments one or more methods for producing one or more RNA stabilizing composition described herein may comprise producing an RNA stabilizing composition that may comprise a defined minimum ratio (e.g. RNA:cellular uptake agent) of the total amount of all RNA or RNA substances to the total amount of one or more cellular uptake agents (as measured on a weight-by-weight basis) in an RNA stabilizing composition as described herein. As a non-limiting example, a method may comprise combining, such as by mixing, one or more RNA stabilizing substance with at least one or more RNA substance to produce an RNA stabilizing composition (such as a pharmaceutical composition, as a non-limiting example) comprising at least one or more RNA substance and at least one or more RNA stabilizing substance, wherein a composition may comprise a defined minimum ratio (e.g. RNA:lipid) of the total amount of all RNA or RNA substances to the total amount of all lipids (as measured on a weight-by-weight basis) in an RNA stabilizing composition. As a non-limiting example, a method for producing an RNA stabilizing composition (such as a pharmaceutical composition, as non-limiting example), may comprise producing a composition that may comprise a defined minimum ratio (e.g. RNA:lipid) of the total amount of all RNA or RNA substances to the total amount of all lipids (as measured on a weight-by-weight basis) in an RNA stabilizing composition for administering at least one or more pharmaceutically active RNA substance to a subject in need thereof.

### Providing Compositions

Another non-limiting example method provides at least one RNA stabilizing composition. RNA stabilizing compositions described herein may be provided as compositions comprising at least one RNA substance and at least one RNA stabilizing substance. Additionally, RNA stabilizing compositions may be provided as one or more products or compositions comprising at least one RNA substance. As a non-limiting example, an RNA stabilizing composition may be provided as a pharmaceutical composition comprising at least one RNA substance for later use.

Non-limiting example methods for providing an RNA stabilizing composition may comprise one or more method steps described herein. Non-limiting example steps in a method may include providing an RNA stabilizing composition comprising at least one RNA substance for later use, wherein a method may comprise at least one of packaging, shipping, or storing an RNA stabilizing composition.

Non-limiting example methods of the present disclosure may comprise one more steps that may be at least part of one or more method described herein. As a non-limiting example, one or more method may comprise one or more steps or one or more combinations of steps from one or more method described herein. As a non-limiting example, one or more method steps for producing an RNA stabilizing composition may be combined with one or more method steps for providing an RNA stabilizing composition, wherein a method may comprise at least one of producing or providing an RNA stabilizing composition comprising one or more method steps described herein. As a non-limiting example, one or more methods may also comprise at least one of producing or providing a kit comprising at least one component of an RNA stabilizing composition that may be produced or provided as an individual component and then combined later to produce an RNA stabilizing composition comprising at least one RNA substance and at least one RNA stabilizing substance.

RNA stabilizing compositions may be provided in one or more chamber described herein, non-limiting examples include vials, syringes, tubes, or multi-compartment syringes. A chamber may be hermetically sealed or also include one or more label as described herein. A label on a chamber may comprise information about the contents of the chamber including, product name, dosing information, instructions for use, storage conditions, manufacturing date, or expiration date or other suitable information described herein. A chamber may be provided as a hermetically sealed chamber or may also be used to provide one or more additional components, such as a diluent for mixing with or reducing viscosity of a composition prior to use. A method may comprise at least one of storing or packaging one or more chambers containing an RNA stabilizing composition or one or more additional components provided with an RNA stabilizing composition. A method comprising packaging an RNA stabilizing composition, may comprise packaging one or more chamber into a suitable package, such as a walled container with a moveable cover or box, or bag, as non-limiting examples. A package containing at least one RNA stabilizing composition or additional component, may also comprise a label with information about the contents of the package or storage or shipping conditions or other suitable information. A method may also comprise sealing a package, such as with an adhesive seal or mechanical interference such as a latch or tab or other suitable method described herein, prior to at least one of storage, shipping, or transport. A method for providing an RNA stabilizing composition may comprise transporting, shipping, or storing a package containing at least one RNA stabilizing composition, wherein a package may be transported or shipped to a desired location, such as a location of use, or stored at a location of use. A method for providing an RNA stabilizing composition may also comprise providing instructions for use. Instructions for use may comprise information for dosing or administration of a composition, storage conditions, or instructions for diluting or mixing a composition with a diluent or other substance prior to the intended use. As a non-limiting example, instructions for use may comprise instructions for mixing an RNA stabilizing composition with one or more other component provided, such as a diluent comprising water, or instructions for mixing two components of a composition in separate compartments of a multi-compartment syringe, as non-limiting examples. A method for providing an RNA stabilizing composition may also comprise providing a composition for an intended use, such as administering a medicament, vaccine, or therapeutic agent to a subject in need thereof if the composition is a pharmaceutical composition, as a non-limiting example. Non-limiting uses of a composition may include administering a pharmaceutical composition to a subject in need thereof, or applying a biostimulant composition to a crop, soil, or other desired area, or implanting an embedded complex comprising an implant into a subject, as non-limiting examples.

A non-limiting example method for providing a pharmaceutical composition, may comprise providing a chamber containing a composition comprising at least one RNA stabilizing substance and at least one pharmaceutically active RNA substance. A composition may also comprise one or more cellular uptake agent or additional substances described herein, as non-limiting examples. The chamber may be a vial, syringe, tube, or multi-compartment syringe, as non-limiting examples. A chamber may be provided as a hermetically sealed chamber containing the pharmaceutical composition. The chamber may also include a label with dosing information, product name, storage conditions, expiration date, instructions for administration or other information described herein. As a non-limiting example, one or more additional components may also be provided, such as a diluent comprising water for reducing viscosity of a composition prior to use. A method may also comprise packaging the hermetically sealed chamber containing the pharmaceutical composition into a suitable package, such as a walled container with a moveable cover, or box, or bag, as non-limiting examples. The package containing the pharmaceutical composition or optional additional component, may be sealed and at least one of stored, shipped, or transported as described herein. The package containing the pharmaceutical composition may be transported or shipped to a desired location, such as a location of use, or stored at a location of use. Instructions for use may also be provided, wherein instructions for use may comprise information for dosing or administration of the composition, storage conditions, or optionally instructions for diluting or mixing the composition with a diluent or other substance prior to administration. As a non-limiting example, if one or more component of the pharmaceutical composition is contained in separate compartments of a multi-compartment syringe, then instructions may include instructions for breaking a seal and mixing the substances in the separate compartments prior to use. An intended use of the pharmaceutical composition provided may be administering a medicament, vaccine, or therapeutic agent to a subject in need thereof, as a non-limiting example.

As a non-limiting example, one or more method for providing an RNA stabilizing composition may be used to provide one or more RNA stabilizing composition described herein, including a pharmaceutical composition, biostimulant composition, or an implant as non-limiting examples.

A composition provided for one or more intended use described herein may be administered or applied using one or more methods described herein, non-limiting examples may include using a syringe, inhaler, nasal or mucosal spray, patch, drops, or tablet to administer a pharmaceutical composition; or by mixing a composition with water, fertilizer, or spraying a composition onto soil or desired area to apply a biostimulant composition; or implanting a composition or embedded complex into at least one tissue or organ to administer an implant.

Additionally, a method may also comprise testing for endotoxin, such as using an LAL assay or other method known in the art. As a non-limiting example, an RNA stabilizing composition may be provided with an endotoxin level below a specified value as described herein, such as less than 100 endotoxin units/mL as a non-limiting example.

Additionally, a method may also comprise sterilizing one or more component or composition prior to or after packaging, using one or more suitable sterilization method described herein. Non-limiting examples may include, filter sterilizing, autoclave, steam, heat, radiation, UV, or other suitable method described herein.

Other non-limiting example method steps may also comprise, providing an RNA stabilizing composition that may comprise a defined ratio of RNA substance to cellular uptake agent as measured on a weight-by-weight basis, as described herein. A non-limiting method for providing an RNA stabilizing composition may also comprise providing a composition with a defined minimum ratio of RNA substance to cellular uptake agent as measured on a weight-by-weight basis, such as providing a composition with an RNA substance to cellular uptake agent ratio of at least 1:2 as measured on a weight-by-weight basis.

Non-limiting embodiments of one or more method for providing one or more RNA stabilizing composition described herein, may comprise providing an RNA stabilizing composition comprising at least one or more RNA substance and at least one or more RNA stabilizing substance. As non-limiting example, a method for providing an RNA stabilizing composition, may comprise providing at least one of a pharmaceutical composition, biostimulant composition, or implant, or other RNA stabilizing composition described herein. As a non-limiting example, a method for providing an RNA stabilizing composition may comprise providing a pharmaceutical composition.

Non-limiting embodiments of one or more method for providing one or more RNA stabilizing composition described herein, may comprise providing a composition for at least one of administering or delivering at least one or more RNA substance to at least one of a cell, organ, tissue, plant, insect, animal, mammal, human, or subject in need thereof. As a non-limiting example, a method may comprise providing a pharmaceutical composition for administering at least one or more pharmaceutically active RNA substance to a subject in need thereof (such as to prevent or treat one or more disease or medical indications, as non-limiting examples).

As a non-limiting example one or methods for providing an RNA stabilizing composition may comprise providing an RNA stabilizing composition comprising at least one or more RNA stabilizing substance and at least one or more RNA substance, and the composition may also comprise one or more additional substances described herein (such as one or more cellular uptake agents, additional RNA stabilizing substances, additional RNA substances, additive substances, inorganic cations (including salts thereof), buffering agents, solvents, or water, or one or more other substances described herein, as non-limiting examples).

In other embodiments one or more methods for providing one or more RNA stabilizing composition described herein may comprise providing an RNA stabilizing composition that may comprise a defined minimum ratio (e.g. RNA:cellular uptake agent) of the total amount of all RNA or RNA substances to the total amount of one or more cellular uptake agents (as measured on a weight-by-weight basis) in an RNA stabilizing composition as described herein. As a non-limiting example, a method may comprise providing an RNA stabilizing composition (such as a pharmaceutical composition, as a non-limiting example), wherein the composition may comprise a defined minimum ratio (e.g. RNA:lipid) of the total amount of all RNA or RNA substances to the total amount of all lipids (as measured on a weight-by-weight basis) in the RNA stabilizing composition. As a non-limiting example, a method for providing an RNA stabilizing composition may comprise providing a composition with a defined minimum ratio of RNA to cellular uptake agent for at least one of administering or delivering at least one or more RNA substance to at least one of a cell, organ, tissue, plant, insect, animal, mammal, human, or subject in need thereof, as non-limiting examples. As another non-limiting example, a method may comprise providing an RNA stabilizing composition (such as a pharmaceutical composition, as non-limiting example) that may comprise a defined minimum ratio (e.g. RNA:lipid) of the total amount of all RNA or RNA substances to the total amount of all lipids (as measured on a weight-by-weight basis) in the RNA stabilizing composition for administering at least one or more pharmaceutically active RNA substance to a subject in need thereof.

### RNA Stabilizing Composition Product Kits

As a non-limiting example, one or more components of one or more RNA stabilizing composition described herein, may be stored separately or may be stored as combinations of one or more components, such as in a kit and then combined later prior to use. As a non-limiting example, one or more methods may also comprise one or more steps for at least one of producing or providing a kit comprising at least one component of an RNA stabilizing composition that may be produced or provided as one or more separate components and then combined later to produce and RNA stabilizing composition comprising at least one RNA substance and at least one RNA stabilizing substance.

Non-limiting example methods for at least one of producing or providing a kit comprising one or more RNA stabilizing composition components may comprise assembling one or more kit components, such as placing one or more components into a chamber (e.g. syringe, tube, or vial) and sealing said chamber (such as hermetically sealing or sealing with a resealable cover, such as friction-fit cap, screw-cap, plug, or lid, as non-limiting examples). A chamber may have a label with information about the contents of the chamber, such as name of components, storage or dosing information, or instructions for use, or alpha-numeric code, or other information or instructions as described herein. The kit components may then be packaged by placing the chambers containing each component into a suitable package, such as a box, bag, or walled container, as described herein. Additionally, a package may comprise a label with information about the contents of the package or other suitable information as described herein. A method may also comprise sealing the package containing the kit components, such as with an adhesive seal or mechanical interference, such as a latch or tab, or other suitable method described herein, prior to at least one of storage, shipping, or transport. A method may also comprise at least one of transporting, shipping, or storing a package containing at least one or more kit components, wherein a package may be transported or shipped to a desired location, such as a location of use, or stored at a location of use. A method may also comprise providing instructions for use. As a non-limiting example, instructions for use as described herein, may be packaged and provided in the package containing the kit components (such as including hardcopy instructions in the package), or instructions for use may also be provided electronically (such as on the internet or pdf) or by one or more other method described herein. Instructions for use may comprise instructions for mixing one or more kit components, such as mixing a diluent or cellular uptake agent with an RNA stabilizing composition prior to use, or instructions for dosing or administration of a composition, or storage conditions, or other suitable instructions as described herein.

As a non-limiting example, a kit may comprise one or more RNA stabilizing substance, RNA substance, cellular uptake agents, or one or more additional components, such as a diluent for diluting or reducing the viscosity of one or more components or compositions prior to use. A kit may comprise components that may be stored separately as individual substances or may be stored as combinations or mixtures of two or more substances, such as a combination of one or more RNA stabilizing substance and one or more RNA substance, that may then be combined with one or more cellular uptake agents prior to use, as a non-limiting example.

As a non-limiting example, one or more component substances of an RNA stabilizing composition (such as a pharmaceutical composition, as a non-limiting example), may be stored separately in separate compartments of a multi-compartment syringe, and then combined prior to use to produce a pharmaceutical composition comprising one or more additional component substances.

Non-limiting example methods of use of a kit comprising one or more RNA stabilizing composition component may include combining one or more kit components to produce one of a pharmaceutical composition, biostimulant composition, or implant. As a non-limiting example one or more kit components may be combined to produce a pharmaceutical composition comprising a pharmaceutically active RNA substance that may be administered to a subject in need thereof. Non-limiting methods of use of a composition may include administering a pharmaceutical composition to a subject in need thereof, or applying a biostimulant composition to a crop, soil, or other desired area, or implanting an embedded complex comprising an implant into a subject, as non-limiting examples.

As a non-limiting example one or more components of a composition may be stored in separate compartments of a multi-compartment syringe, wherein the components may be mixed, such as by breaking a seal separating the two components of the composition prior to use. As a non-limiting example, one compartment may contain an RNA stabilizing composition with a selectively adjustable viscosity and a second compartment may contain a diluent (such as a buffer or water) that when mixed with the RNA stabilizing composition may reduce the viscosity to a specified value or range prior to use. As another non-limiting example, one compartment may contain an RNA stabilizing composition and a second compartment may contain one or more cellular uptake agent that may be mixed with the RNA stabilizing composition prior to use (as a non-limiting example, when mixed one or more cellular uptake agent may at least partially complex with or at least partially encapsulate at least one or more RNA substance). A non-limiting example method of use may comprise a multi-compartment syringe containing at least two separate components of a pharmaceutical composition that may be mixed prior to administering at least one of a medicament, vaccine, or therapeutic agent to a subject in need thereof.

As non-limiting examples, at least one method may comprise at least one step of producing, packaging, transporting, shipping, or storing, or using compositions comprising one or more RNA stabilizing substance and one or more RNA substance. Methods comprising using at least one RNA stabilizing substance may employ at least one RNA stabilizing substance for producing one or more products or compositions comprising at least one RNA substance for later use. As a non-limiting example, an RNA stabilizing substance may be used to produce a pharmaceutical composition comprising at least one RNA substance for later use. Methods comprising using at least one RNA stabilizing substance may also employ at least one RNA stabilizing substance for laboratory use or diagnostic uses, such as sequencing or analysis of one or more RNA substance. One or more methods of the present disclosure may comprise at least one of packaging, transporting, shipping, storing, or using compositions comprising one or more RNA stabilizing substance and one or more RNA substance for extended durations after compositions are produced. As non-limiting examples, extended durations may be at least 4 hours, 12 hours, 24 hours, 48 hours, 72 hours, 7 days, 14 days, 30 days, 60 days, 3 months, 6 months, 12 months, 18 months, or 24 months. As a non-limiting example, one method of the present disclosure may be using a composition comprising at least one RNA stabilizing substance and at least one RNA substance as a medicament that may be administered to provide one or more therapies an extended duration after the composition was produced.

As a non-limiting example, one or more method for providing an RNA stabilizing composition or a kit comprising at least one or more RNA stabilizing composition components may comprise providing instructions for use comprising instructions for using at least one RNA stabilizing composition or mixing or combining at least one provided component such as a diluent, RNA stabilizing substance, or cellular uptake agent, or mixing components in separate compartments of a multi-compartment syringe, as non-limiting examples. Instructions for use may include instructions for mixing or diluting one or more components prior to use, such as mixing a diluent or cellular uptake agent with an RNA stabilizing composition, or instructions for use may include instructions for storage or dosing information, or instructions for reducing the viscosity of a provided composition or components. As a non-limiting example, instructions for use may comprise instructions for mixing a diluent comprising water (such as a buffer or water) with a composition to reduce viscosity prior to use. As non-limiting examples, the instructions may be provided with an RNA stabilizing composition or kit component (such as, in printed or electronically readable or digital form) or may be made available remotely (such as in transmitted or downloadable electronic form or digital form).

Additionally, an RNA stabilizing composition kit may also comprise one or more component with an endotoxin level below a specified value, wherein a method may comprise performing endotoxin testing of one or more components, such as using an LAL assay as described herein. As a non-limiting example, one more kit components may be provided with an endotoxin level below a specified value as described herein, such as less than 100 endotoxin units/mL as a non-limiting example.

Additionally, an RNA stabilizing composition kit may also comprise one or more sterilized component or composition that may be sterilized prior to or after packaging, using one or more suitable sterilization method described herein. Non-limiting examples may include, filter sterilizing, autoclave, steam, heat, radiation, UV, or other suitable method described herein.

In some embodiments one or more component substances of an RNA stabilizing composition described herein (such as a pharmaceutical composition, as a non-limiting example), may be stored separately (such as in a kit or in compartments of a multi-compartment syringe described herein, as non-limiting examples) and then combined later, prior to use to produce an RNA stabilizing composition comprising one or more RNA substance and one or more RNA stabilizing substance.

Non-limiting embodiments of one or more method for providing one or more RNA stabilizing composition may comprise providing one or more component substances of an RNA stabilizing composition described herein (such as a pharmaceutical composition, as a non-limiting example) for producing a composition comprising at least one or more RNA substance and at least one or more RNA stabilizing substance, wherein one or more component may be stored separately (such as in a kit or in compartments of a multi-compartment syringe described herein, as non-limiting examples) and then combined later prior to use to produce an RNA stabilizing composition comprising one or more RNA substance and one or more RNA stabilizing substance. As a non-limiting example, a method may comprise providing one or more component substances to produce a pharmaceutical composition comprising one or more RNA substance and one or more RNA stabilizing substance, wherein each component may be stored separately as individual substances or as mixtures or combinations of substances, and combined prior to use (such as administering one or more RNA substance to a subject in need thereof, as a non-limiting example) to produce a composition comprising one or more RNA substance and one or more RNA stabilizing substance.

In some embodiments one or more component substances of an RNA stabilizing composition may be stored individually or as a combination or mixture of two or more substances (such as 2 or more, or 3 or more, or 4 or more, or 5 or more, or 6 or more substances). As a non-limiting example, one or more, or 2 or more, or 3 or more, or 4 or more, or 5 or more, or 6 or more component substances of an RNA stabilizing composition may be stored individually or as combinations of two or more substances, as non-limiting examples. In other embodiments one or more components may include one or more additional substances described herein (such as one or more cellular uptake agents, additional RNA stabilizing substances, additional RNA substances, additive substances, inorganic cations (including inorganic or organic salts comprising one or more inorganic cation), buffering agents, water, solvents, or one or more other substances described herein, as non-limiting examples). Non-limiting example uses of an RNA stabilizing composition produced by combining one or more separate components may include at least one of administering or delivering at least one or more RNA substance to at least one of a cell, organ, tissue, plant, insect, animal, mammal, human, or subject in need thereof.

### Storage and Stability

Non-limiting examples of steps in one or more method of the present disclosure may comprise one more steps where an RNA stabilizing composition may be stored at a defined temperature for a defined period of time. As a non-limiting example, one or more method may comprise one or more steps or one or more combinations of steps where an RNA stabilizing composition may be stored at a defined temperature for a defined period of time, such as storing an RNA stabilizing composition in a chamber for a defined duration or minimum period of time at a defined minimum temperature. A non-limiting example step in one or more method for providing an RNA stabilizing composition may comprise storing the composition in a chamber at a defined minimum temperature for a defined minimum period of time, such as storing the composition at a minimum temperature of at least 20° C. for at least 7 days, as a non-limiting example. As a non-limiting example, a step in one or more method for providing an RNA stabilizing composition may comprise providing an RNA stabilizing composition in a hermetically sealed chamber that is packaged and stored at a defined minimum temperature for a defined period of time. Non-limiting example methods of use may also comprise maintaining a defined level of RNA stability at a defined temperature for a defined duration or period of time. As a non-limiting example, a step in one or more a method for providing an RNA stabilizing composition may also comprise storing a composition in a chamber at a defined minimum temperature for a defined duration or period of time, wherein the RNA substance maintains a defined level of stability as described herein.

Other non-limiting example methods of use may also comprise, storing an RNA stabilizing composition comprising one or more cellular uptake agent at a defined temperature for a defined duration or period of time, wherein a particle or complex comprising at least one cellular uptake agent and at least one RNA substance maintains a defined size distribution or maintains a defined level of particle or complex stability. As a non-limiting example, a method for providing an RNA stabilizing composition may also comprise storing a composition comprising a particle or complex comprising at least one cellular uptake agent and at least one RNA substance at a defined minimum temperature for a defined duration or period of time, wherein the particles or complex maintain a defined size distribution; or retain a defined percentage of RNA remaining encapsulated within the particles or complexed to the particles, as described herein.

In some embodiments of the present disclosure one or more method for at least one of producing or providing one or more RNA stabilizing composition described herein (such as a pharmaceutical composition, as a non-limiting example) comprising one or more RNA substance and one or more RNA stabilizing substance, may comprise one or more steps of storing a composition in a chamber at a defined minimum temperature for a defined duration of time. As a non-limiting example, a method may comprise storing an RNA stabilizing composition (such as a pharmaceutical composition, as a non-limiting example) in one or more chamber described herein, at a defined minimum temperature (such as at least about 20° C. or greater, as a non-limiting example) for a defined duration of time between a minimum time and a maximum time wherein the minimum time may be at least one of 1 hour, 24 hours, 48 hours, 72 hours, 100 hours, 7 days, 14 days, 30 days, 60 days, 3 months, 6 months, 12 months, 18 months, or 24 months and the maximum time is greater than the minimum time and may be at least one of 6 hours, 24 hours, 48 hours, 72 hours, 100 hours, 7 days, 14 days, 30 days, 60 days, 3 months, 6 months, 12 months, 18 months, 24 months, 48 months, 5 years, 10 years, or 20 years. As non-limiting examples the exposure to temperatures of at least the defined minimum temperature may be continuous or the exposure may be intermittent. As another non-limiting example, a method may comprise storing an RNA stabilizing composition (such as a pharmaceutical composition, as a non-limiting example) in one or more chamber described herein, at a defined minimum temperature of at least about 20° C. or greater for a defined duration between at least 1 hour to 5 years.

In some embodiments of the present disclosure one or more method for at least one of producing or for providing one or more RNA stabilizing composition described herein (such as a pharmaceutical composition, as a non-limiting example) comprising one or more RNA substance and one or more RNA stabilizing substance, may also comprise one or more steps of storing a composition in a chamber at a defined minimum temperature for a defined minimum period of time, wherein the defined minimum temperature may be at least −20° C., −10° C., 0° C., 4° C., 10° C., 15° C., 20° C., 30° C., 40° C., or 50° C., and the defined minimum period of time may be at least 1 hour, 2 hours, 4 hours, 8 hours, 12 hours, 18 hours, 24 hours, 48 hours, 72 hours, 100 hours, 7 days, 14 days, 30 days, 60 days, 3 months, 6 months, 12 months, 18 months, or 24 months. As non-limiting examples, the exposure to temperatures of at least the defined minimum temperature may be continuous or the exposure may be intermittent. As another non-limiting example, a method may comprise storing an RNA stabilizing composition (such as a pharmaceutical composition, as a non-limiting example) in one or more chamber described herein at a defined minimum temperature (such as at least 15° C.) for a defined minimum period of time (such as at least 30 days), wherein the exposure to temperatures of at least the defined minimum temperature may be continuous or the exposure may be intermittent.

In other embodiments one or more method described herein comprising one or more steps of storing one or more RNA stabilizing composition (e.g. a pharmaceutical composition) at a defined temperature for a defined period of time, may further comprise maintaining a defined level of RNA stability at a defined temperature for a defined period of time as described herein. As a non-limiting example RNA stability of an RNA substance may be determined by comparing the starting average molecular weight of a sample of the RNA substance to the average molecular weight of a sample of the RNA substance that has been stored for at least one predetermined time and temperature. The stability of an RNA substance may be determined by exposing the RNA substance to a specified temperature for a specified time duration and comparing the ending average molecular weight to the starting molecular weight to determine the amount of degradation. The amount of degradation of an RNA substance between the beginning and the end of a time interval is calculated using the formula D=[1−(AMWe/AMWs)]*100 as described herein. AMWe is average molecular weight at end, AMWs is average molecular weight at start.

As non-limiting example embodiments of the present disclosure, an RNA stabilizing composition comprising at least one RNA substance and at least one RNA stabilizing substance may stabilize the RNA substance such that the amount of degradation (D) of the RNA substance (defined as D=[1−(AMWe/AMWs)]*100) is less than about X % with temperatures exceeding a defined minimum temperature of about T° C. where X may be less than about 5%, 10%, 15%, 20%, 25%, 30%, 35%, 40%, or 50%; and T may be about −80° C., −60° C., −40° C., −30° C., −20° C., −10° C., 0° C., 2° C., 4° C., 6° C., 8° C., 10° C., 15° C., 20° C., 25° C., 30° C., 40° C., or 50° C.; for at least one of about 1 hour, about 2 hours, about 4 hours, about 8 hours, about 12 hours, about 18 hours, about 24 hours, about 48 hours, about 72 hours, about 100 hours, about 7 days, about 14 days, about 30 days, about 60 days, about 3 months, about 6 months, about 12 months, about 18 months, or about 24 months. As non-limiting examples the exposure to temperatures of at least the defined temperature may be continuous or the exposure may be intermittent. As a non-limiting example, a method may comprise storing an RNA stabilizing composition (such as a pharmaceutical composition, as a non-limiting example) in one or more chamber described herein at a defined minimum temperature (such as at least 15° C.) for a defined minimum period of time (such as at least 30 days), wherein the amount of RNA degradation may be less than about 5%, 10%, 15%, 20%, 25%, 30%, 35%, 40%, or 50%. As another non-limiting example, a method may comprise storing an RNA stabilizing composition (such as a pharmaceutical composition, as a non-limiting example) in one or more chamber described herein at a defined minimum temperature of at least 20° C. for a defined minimum period of time of at least 3 months, wherein the amount of RNA degradation may be less than about 10%.

In other embodiments one or more method described herein comprising one or more steps of storing one or more RNA stabilizing composition (e.g. a pharmaceutical composition) at a defined temperature for a defined period of time, may further comprise a composition wherein a particle or complex comprising one or more cellular uptake agent and one or more RNA substance maintains a specified size distribution for a defined minimum period of time at a defined minimum temperature as described herein. As a non-limiting example, a method may comprise storing an RNA stabilizing composition (e.g. a pharmaceutical composition) in a chamber at a defined minimum temperature for a defined minimum period of time, wherein the composition comprises a particle or complex comprising at least one or more cellular uptake agent and at least one or more RNA substance that maintains a specified size distribution for a defined minimum period of time at a defined minimum temperature as described herein

In other embodiments one or more method described herein comprising storing one or more RNA stabilizing composition (e.g. a pharmaceutical composition) at a defined temperature for a defined period of time, may further comprise a composition wherein a particle or complex comprising one or more cellular uptake agent and one or more RNA substance retains a specified minimum level of RNA encapsulated within a particle or RNA complexed to a particle for a defined minimum period of time at a defined minimum temperature as described herein. As a non-limiting example, a method may comprise storing an RNA stabilizing composition (e.g. a pharmaceutical composition) in a chamber at a defined minimum temperature for a defined minimum period of time, wherein a particle or complex comprising one or more cellular uptake agent and one or more RNA substance retains a specified minimum level of RNA encapsulated within a particle or RNA complexed to a particle for defined minimum period of time at a defined minimum temperature as described herein.

### Additional Uses and Applications

Non-limiting methods of use may include using one or more RNA stabilizing composition described herein to at least one of administer or deliver at least one or more RNA substance to at least one of a cell, organ, tissue, plant, insect, animal, mammal, human, or subject in need thereof. As a non-limiting example, a method of use may comprise using an RNA stabilizing composition, wherein an RNA stabilizing composition may be a pharmaceutical composition, to at least one of administer or deliver at least one or more RNA substance to a subject in need thereof (such as to prevent or treat one or more disease or medical indications, as non-limiting examples).

As used herein, a subject in need thereof may be an animal (e.g. chicken, duck, or bird, as non-limiting examples), mammal (e.g. cow, horse, pig, dog, or cat, as non-limiting examples), non-human primate, or human, wherein an RNA stabilizing composition, such as a pharmaceutical composition, may be at least one of administered or used to at least one of prevent, treat, or diagnose one or more diseases or medical indications or otherwise improve or prolong the health of the subject.

Non-limiting examples of medical indications or diseases may include, infectious diseases (including infectious diseases caused by a virus, bacteria, protozoa, parasite, or fungi), cancer, tumors (including malignant or non-malignant tumors), respiratory diseases, cognitive diseases (e.g. Alzheimer's disease as a non-limiting example), metabolic diseases, cardiovascular diseases, skin diseases, ophthalmic diseases, immune diseases, autoimmune diseases, allergies, muscular diseases, or arthritis (e.g. rheumatoid arthritis as a non-limiting example) or joint diseases.

In some embodiments one or more RNA stabilizing composition described herein (e.g. a pharmaceutical composition, as a non-limiting example) may be administered via one or more route of administration including but not limited to oral, sublingual, transdermal, ophthalmic, parenteral, subcutaneous, intravenous, intramuscular, by inhalation, topical, rectal, nasal, buccal, vaginal, or via an implant.

In some embodiments one or more RNA stabilizing composition described herein (such as a pharmaceutical composition, as a non-limiting example) may comprise one or more RNA substance comprising a coding RNA, wherein the coding RNA may comprise a translatable region encoding a polypeptide of interest. As a non-limiting example, one or more RNA stabilizing composition described herein may comprise an RNA substance comprising a coding RNA, wherein the RNA stabilizing composition may be at least one of administered or delivered to at least one of a cell, organ, tissue, plant, insect, animal, mammal, human, or subject in need thereof, wherein the coding RNA may produce a polypeptide of interest. As another non-limiting example, one or more pharmaceutical composition described herein may comprise an RNA substance comprising a coding RNA, wherein the pharmaceutical composition may be administered to a subject in need thereof, wherein the coding RNA may produce a polypeptide of interest (such as, a vaccine immunogen, or antibody, to prevent or treat one or more disease or medical indications, as non-limiting examples).

In some embodiments one or more RNA stabilizing composition described herein (such as a pharmaceutical composition, as a non-limiting example) may comprise one or more RNA substance comprising a non-coding RNA, wherein the non-coding RNA may comprise an at least partially complementary RNA strand to one or more target DNA or RNA sequence. As a non-limiting example, one or more RNA stabilizing composition described herein may comprise an RNA substance comprising a non-coding RNA, wherein the RNA stabilizing composition may be at least one of administered or delivered to at least one of a cell, organ, tissue, plant, insect, animal, mammal, human, or subject in need thereof, wherein the non-coding RNA may decrease the expression of one or more proteins (such as an essential protein in an insect or pathogen, or a misfolded, mutant, or non-functional protein in a subject in need thereof, as non-limiting examples). As another non-limiting example, one or more pharmaceutical composition described herein may comprise an RNA substance comprising a non-coding RNA, wherein the pharmaceutical composition may be administered to a subject in need thereof, wherein the non-coding RNA may decrease the expression of one or more proteins (such as a misfolded, mutant, or non-functional protein, to prevent or treat one or more disease or medical indications, as non-limiting examples).

In some embodiments one or more method described herein may also comprise providing instructions for use. As a non-limiting example, instructions for use may comprise instructions for mixing, diluting, or combining one or more substances or compositions, such as mixing or diluting a composition with one or more component prior to use. As a non-limiting example, instructions for use may comprise information for mixing one or more buffer or diluent with one or more RNA stabilizing composition to adjust the viscosity, or adjust the pH, or adjust the concentration prior to use. Instructions for use may also comprise instructions for storage conditions, such as storage temperature, or instructions for administering, or dosing information for one or more RNA stabilizing composition (such as a pharmaceutical composition, as a non-limiting example).

As described herein, instructions for use may comprise written instructions comprising one or more sheets of material separate from a chamber or packaging, or may comprise sheets of material inserted into or attached to packaging, or attached to one or more chambers (such as a label providing instructions for use attached to a chamber). As a non-limiting example, instructions for use may comprise information, such as instructions comprising words or illustrations. As a non-limiting example, instructions for use may be provided as one or more sheets or labels comprising paper or plastic, or other suitable material, or may be provided electronically (such as on the internet, or a pdf, or other suitable electronic method).

In some embodiments one or more method described herein may also comprise providing one or more RNA stabilizing composition in a chamber, wherein a chamber may have at least one or more label. Labels on chambers may include information about the substance or composition within the chamber, such as a name or identifier of the substance or composition in the chamber. As non-limiting examples, labels on chambers may include instructions for use or relevant manufacturing or storage information such as lot numbers, trace codes, expiration dates, storage conditions (e.g. storage temperature), manufacturing date, or manufacturer or manufacturing location. Labels on chambers may also include dosing information or instructions for administering one or more RNA stabilizing composition (such as a pharmaceutical composition, as a non-limiting example).

In some embodiments one or more method described herein may comprise providing one or more RNA stabilizing composition or other substances as at least part of a kit, wherein one or more components of a composition may be stored separately and then combined prior to use as described herein. As a non-limiting example, a method may comprise storing one or more substances or components in a composition separately (such as a buffer, diluent, or one or more cellular uptake agents) in a multi-compartment syringe and then combining the components prior to use. As another non-limiting example, a method may comprise providing one or more substance or component (such as a buffer, diluent, or one or more cellular uptake agents) to mix or otherwise combine with one or more RNA stabilizing composition prior to use.

In some embodiments one or more method described herein may comprise shipping, transporting, or storing a package containing one or more RNA stabilizing composition or component. As non-limiting examples, a package may be transported or shipped by airplane, automobile (such as truck, van, or car as non-limiting examples) or boat, or combinations thereof. In some embodiments a package may be transported, shipped, or stored at about 20° C. or greater. In some embodiments a package may be transported, shipped, or stored at about −20° C. or greater, or about −10° C. or greater, or about 0° C. or greater, or about 4° C. or greater, or about 10° C. or greater, or about 20° C. or greater, or about 30° C. or greater.

One of ordinary skill in the art would appreciate that one or more examples, methods, descriptions, or compositions (e.g. compositions comprising one or more RNA substance and one or more RNA stabilizing substance as non-limiting examples) described herein may be combined to create a kit, method, process, or composition that may include one or more elements or steps from one or more examples, methods, descriptions, or compositions of the present disclosure.

Other non-limiting embodiments of the present disclosure may include combinations of one or more examples, methods, descriptions, or compositions (e.g. compositions comprising one or more RNA substance and one or more RNA stabilizing substance as non-limiting examples) described herein to create a kit, method, process, or composition that may include one or more elements or steps from one or more examples, methods, descriptions, or compositions described herein.

In some embodiments, one or more method described herein may be combined with one or more other methods described herein, such as one or more methods for producing or for providing one or more RNA stabilizing composition or producing or providing one or more components of one or more kits as non-limiting examples.

As a non-limiting example, a method may comprise producing one or more RNA stabilizing compositions described herein (such as a pharmaceutical composition as a non-limiting example) wherein the composition may have a selectively adjustable viscosity; wherein the method may be combined with a method that may comprise providing one or more RNA stabilizing composition in a chamber (such as a multicompartment syringe) and providing instructions use, such as instructions for diluting or mixing one or more additional components with the composition prior to use. As a non-limiting example, the above method may also comprise providing one or more kit components, such as a diluent (e.g. a diluent comprising water, such as a buffer or water), or may also comprise providing a chamber with a label comprising relevant storage, dosing, or manufacturing information (such as a lot number, production date, or expiration date, as a non-limiting example).

As another non-limiting example, a method may comprise producing one or more RNA stabilizing composition described herein (such as a pharmaceutical composition as a non-limiting example) wherein a method may comprise producing a composition by combining an exogenous mono-nucleotide substance with a polymeric RNA substance after the RNA has been at least one of synthesized or at least partially purified, to produce an RNA stabilizing composition and placing the composition in a chamber; wherein the method may also comprise providing a chamber wherein the chamber may be stored at a defined minimum temperature for a defined period of time prior to use, or may also comprise maintaining a defined level of RNA stability after a defined minimum period of time at the defined minimum temperature. As a non-limiting example, the above method may also comprise providing instructions for use or providing the composition in a chamber with a label as described herein.

Non-limiting examples of one or more pharmaceutically active RNA substances are described in the following: U.S. Pat. Nos. 10,898,574; 10,702,600; 8,217,016; 10,064,934; 11,643,441; 10,738,355; 10,485,884; and US Patent Application No. US 2020/0254086, each incorporated herein by reference. As a non-limiting example, one or more pharmaceutical composition may comprise one or more pharmaceutically active RNA substance, wherein a pharmaceutical composition may be used to at least one of prevent, treat, or diagnose one or more diseases or medical indications (such as one or more infectious diseases, cancer, tumors, respiratory diseases, or cardiovascular diseases as non-limiting examples).

In a further aspect, the present disclosure further provides the use of the inventive method for producing a pharmaceutical composition.

In some embodiments of the present disclosure, one or more RNA stabilizing compositions may be used to produce a pharmaceutical composition.

According to yet another aspect of the present disclosure, a pharmaceutical composition may be provided, wherein a pharmaceutical composition may comprise one or more RNA stabilizing compositions described herein.

As used herein, a pharmaceutical composition comprises an RNA stabilizing composition that further comprises at least one pharmaceutically active RNA substance. As a non-limiting example, a pharmaceutically active RNA substance may be used to at least one of prevent, treat, or diagnose one or more diseases or medical indications or improve or prolong the health of humans, or vertebrate animals, including non-human primates, mammals, and birds.

In some embodiments a pharmaceutical composition may comprise at least one of a medicament, vaccine, or therapeutic agent. In some embodiments a pharmaceutical composition may comprise at least one of a coding RNA or non-coding RNA.

In some embodiments a pharmaceutical composition may comprise one or more RNA substance that comprises a pharmaceutically active RNA. In some embodiments a pharmaceutical composition may comprise one or more RNA substance that may be an active pharmaceutical ingredient. In some embodiments a pharmaceutical composition may comprise at least one or more pharmaceutically active RNA component (such as a pharmaceutically active RNA substance). In some embodiments, a pharmaceutical composition may comprise one or more additional pharmaceutically acceptable ingredient, such as a pharmaceutically acceptable carrier or vehicle.

In some embodiments a pharmaceutical composition may comprise one or more RNA stabilizing composition described herein comprising a combination or mixture of at least one or more RNA stabilizing substance and at least one or more RNA substance. In some embodiments a pharmaceutical composition may comprise one or more RNA stabilizing composition described herein comprising a combination or mixture of at least one or more RNA stabilizing substance, at least one or more RNA substance, and at least one or more cellular uptake agent. In other non-limiting embodiments, a pharmaceutical composition may also comprise one or more additional substances described herein including, one or more cellular uptake agents, additional RNA stabilizing substances, additional RNA substances, additive substances, inorganic cations (including salts thereof), buffering agents, or water, as non-limiting examples.

In some embodiments a pharmaceutical composition may comprise one or more non-RNA pharmaceutically active component. Wherein a non-RNA pharmaceutically active component may be a compound that has a therapeutic effect against a particular medical indication, such as, but not limited to, cancer diseases, autoimmune disease, allergies, or infectious diseases as non-limiting examples. Non-limiting examples of such compounds may include, but are not limited to: peptides or proteins, (therapeutically active) low molecular weight organic or inorganic compounds (molecular weight less than 5,000), sugars, antigens or antibodies, therapeutic agents already known in the art, antigenic cells, antigenic cellular fragments, cellular fractions, modified, attenuated or de-activated pathogens (e.g. virus, bacteria, fungus, protozoa, plasmodium, or mycobacterium), wherein a pathogen may be attenuated or deactivated chemically, by irradiation, mutation, serial passage, or other known method.

In some embodiments one or more pharmaceutical compositions may be administered via one or more route of administration including but not limited to oral, sublingual, transdermal, ophthalmic, parenteral, subcutaneous, intravenous, intramuscular, by inhalation, topical, rectal, nasal, buccal, vaginal, or via an implant.

In some methods of use one or more pharmaceutical compositions may be administered via one or more route of administration including but not limited to oral, sublingual, transdermal, ophthalmic, parenteral, subcutaneous, intravenous, intramuscular, by inhalation, topical, rectal, nasal, buccal, vaginal, or via an implant.

As non-limiting example methods of use, one or more pharmaceutical composition may be at least one of produced, provided, administered or used in conjunction with one or more of the following: syringes, prefilled syringes (including multi-compartment syringes), injection, nasal sprays, transdermal patches, eye drops, oral sprays, aerosols, inhalers, nebulizers, oral tablets, pills, sublingual tablets, sublingual drops, suppositories, mucosal sprays, creams, lozenges, lotions, balms, syrups, ointments, implants, mists, or sprays, as non-limiting examples.

In some methods of use one or more RNA stabilizing composition described herein may be used to produce a pharmaceutical composition comprising at least one or more RNA substance and at least one or more RNA stabilizing substance. In some methods of use one or more RNA stabilizing composition described herein may be used to produce a pharmaceutical composition comprising at least one or more RNA substance, at least one or more RNA stabilizing substance, and at least one or more cellular uptake agent.

In some methods of use one or more RNA substance and one or more RNA stabilizing substance described herein may be combined to produce a pharmaceutical composition comprising at least one or more RNA substance and at least one or more RNA stabilizing substance. In some methods of use one or more RNA substance and one or more RNA stabilizing substance, and one or more cellular uptake agent may be combined to produce a pharmaceutical composition comprising at least one or more RNA substance, at least one or more RNA stabilizing substance, and at least one or more cellular uptake agent. In other non-limiting example methods of use, one or more additional substance may be combined with one or more RNA substance and one or more RNA stabilizing substance to produce a pharmaceutical composition, wherein a pharmaceutical composition may also comprise one or more additional substances described herein including, one or more cellular uptake agents, additional RNA stabilizing substances, additional RNA substances, additive substances, inorganic cations (including salts thereof), buffering agents, or water, as non-limiting examples.

Non-limiting examples of one or more biologically active RNA substances are described in the following: U.S. Pat. Nos. 11,891,611; 8,759,306; and US Patent Application Pub. No. US 2009/0285784, each incorporated herein by reference. As a non-limiting example, one or more biostimulant composition may comprise one or more biologically active RNA substance, wherein a biostimulant composition may be used to alter the stress tolerance, reproductive capacity, growth or biological development, feeding or nutrition efficiency of a plant, insect, worm, or fungus, as non-limiting examples.

In a further aspect, the present disclosure further provides the use of the inventive method for producing one or more biostimulant composition.

As used herein, a biostimulant composition comprises an RNA stabilizing composition that further comprises at least one biologically active RNA substance.

As a non-limiting example, a biologically active RNA substance may be used to at least one of alter the stress tolerance (including biotic or abiotic stress), reproductive capacity, growth or biological development, feeding or nutrition efficiency, vitamin production, or mobility of a plant, insect, invertebrate (including worms), fungus, bacteria, algae, or protozoa. As a non-limiting example, a biostimulant composition may include: plant biostimulants (including to improve stress tolerance or nutrition efficiency of crops or agriculture, as non-limiting examples), plant or microbial biocides (including weeds, protozoa, worms, or fungi, as non-limiting examples), pesticides (including insecticides as a non-limiting example), microbial biostimulant, bacterial biostimulant, fungal biostimulant, algae biostimulant, antifungal, antiviral, or insect biostimulant.

As non-limiting examples, a biostimulant composition may comprise an RNA substance that reduces the expression of an essential protein in an insect, worm, or microbe, needed for mobility, nutritional efficiency, or stress tolerance. As another non-limiting example, a biostimulant composition may comprise an RNA substance that increases the production of a plant protein that improves stress tolerance, growth, vitamin production, or nutrition efficiency. As another non-limiting example, a biostimulant composition may comprise an RNA substance that reduces the expression of protein essential for reproduction of an insect, worm, or microbe.

In some embodiments of the present disclosure, one or more RNA stabilizing compositions may be used to produce one or more biostimulant composition, including, but not limited to one or more of the following: plant biostimulants (including for improve stress tolerance or nutrition efficiency of crops or agriculture, as non-limiting examples), plant or microbial biocides (including weeds, protozoa, worms, or fungi, as non-limiting examples), pesticides (including insecticides as a non-limiting example), microbial biostimulant, bacterial biostimulant, fungal biostimulant, algae biostimulant, antifungal, antiviral, or insect biostimulant.

According to yet another aspect of the present disclosure, a biostimulant composition may be provided, wherein a biostimulant composition may comprise one or more RNA stabilizing compositions described herein.

In some embodiments a biostimulant composition may comprise one or more RNA stabilizing composition described herein comprising a combination or mixture of at least one or more RNA stabilizing substance and at least one or more RNA substance. In some embodiments a biostimulant composition may comprise one or more RNA stabilizing composition described herein comprising a combination or mixture of at least one or more RNA stabilizing substance, at least one or more RNA substance, and at least one or more cellular uptake agent. In other non-limiting embodiments, a biostimulant composition may also comprise one or more additional substances described herein including, one or more cellular uptake agents, additional RNA stabilizing substances, additional RNA substances, additive substances, inorganic cations (including salts thereof), buffering agents, or water, as non-limiting examples.

In some embodiments a biostimulant composition may comprise one or more biologically active RNA component (such as a biologically active RNA substance). In some embodiments a biostimulant composition may comprise one or more RNA substance that comprises a biologically active RNA. In some embodiments a biostimulant composition may comprise one or more RNA substance that may be a biologically active ingredient.

In some embodiments a biostimulant composition may comprise at least one of a coding RNA or non-coding RNA.

In some embodiments, a biostimulant composition may comprise one or more additional biologically, environmentally, or agriculturally acceptable ingredient, such as a biologically, environmentally, or agriculturally acceptable carrier or vehicle. In some embodiments a biostimulant composition may comprise one or more non-RNA biologically active component.

In some embodiments one or more biostimulant compositions may be administered or applied via spray, powder, aerosol, mist, solution, water additive, soil additive, fertilizer additive, liquid, tablet, or other known methods.

In some methods of use one or more biostimulant compositions may be administered or applied via spray, powder, aerosol, mist, solution, water additive, soil additive, fertilizer additive, liquid, tablet, or other known methods.

In some methods of use one or more RNA stabilizing composition described herein may be used to produce a biostimulant composition comprising at least one or more RNA substance and at least one or more RNA stabilizing substance. In some methods of use one or more RNA stabilizing composition described herein may be used to produce a biostimulant composition comprising at least one or more RNA substance, at least one or more RNA stabilizing substance, and at least one or more cellular uptake agent.

In some methods of use one or more RNA substance and one or more RNA stabilizing substance described herein may be combined to produce a biostimulant composition comprising at least one or more RNA substance and at least one or more RNA stabilizing substance. In some methods of use one or more RNA substance and one or more RNA stabilizing substance, and one or more cellular uptake agent may be combined to produce a biostimulant composition comprising at least one or more RNA substance, at least one or more RNA stabilizing substance, and at least one or more cellular uptake agent. In other non-limiting example methods of use, one or more additional substance may be combined with one or more RNA substance and one or more RNA stabilizing substance to produce a biostimulant composition, wherein a biostimulant composition may also comprise one or more additional substances described herein including, one or more cellular uptake agents, additional RNA stabilizing substances, additional RNA substances, additive substances, inorganic cations (including salts thereof), buffering agents, or water, as non-limiting examples.

In a further aspect, the present disclosure further provides the use of the inventive method for producing an implant.

As used herein, an implant comprises an RNA stabilizing composition that further comprises at least one pharmaceutically active RNA substance, where the composition is at least partially contained in or at least part of an embedded complex.

As a non-limiting example, an embedded complex may comprise an implant, wherein the implant comprises the volume of implantable material (such as a polymeric material or gel or network) that at least partially contains at least one pharmaceutically active RNA substance. As a non-limiting example, an embedded complex comprising an implant may be implanted or otherwise introduced into a subject to contact at least one of tissues, skin, blood, fluid, or cells of a human or vertebrate animal, wherein the implant may at least partially release or transfer a pharmaceutically active RNA substance into a living organism.

In some embodiments an implant may comprise at least one of a medicament, vaccine, or therapeutic agent. In some embodiments an implant may comprise at least one of a coding RNA or non-coding RNA.

In some embodiments an implant may comprise one or more RNA substance that comprises a pharmaceutically active RNA. In some embodiments an implant may comprise one or more RNA substance that may be an active pharmaceutical ingredient. In some embodiments an implant may comprise at least one or more pharmaceutically active RNA component (such as a pharmaceutically active RNA substance). In some embodiments, an implant may comprise one or more additional pharmaceutically acceptable ingredient, such as a pharmaceutically acceptable carrier or vehicle.

In some embodiments an embedded complex comprising an implant may be implanted by one or more routes or methods as described herein.

In some embodiments of the present disclosure, one or more RNA stabilizing compositions may be used to produce an implant, wherein an implant may be implanted attached or otherwise contacting at least one of tissues, skin, blood, fluid, or cells of a human or vertebrate animal.

According to yet another aspect of the present disclosure, an implant may be provided, wherein an implant may comprise one or more RNA stabilizing compositions as described herein. In some embodiments an implant may comprise a medicament, vaccine, or therapeutic agent.

In some embodiments an implant may comprise one or more composition described herein comprising a combination or mixture of at least one or more RNA stabilizing substance and at least one or more RNA substance. In some embodiments an implant may comprise one or more composition described herein comprising a combination or mixture of at least one or more RNA stabilizing substance, at least one or more RNA substance, and at least one or more cellular uptake agent. In other non-limiting embodiments, an implant may also comprise one or more additional substances described herein including, one or more cellular uptake agents, additional RNA stabilizing substances, additional RNA substances, additive substances, inorganic cations (including salts thereof), buffering agents, or water, as non-limiting examples.

In some embodiments an implant may comprise one or more non-RNA pharmaceutically active component. Wherein a non-RNA pharmaceutically active component may be a compound that has a therapeutic effect against a particular medical indication, such as, but not limited to, cancer diseases, autoimmune disease, allergies, or infectious diseases as non-limiting examples. Non-limiting examples of such compounds may include, but are not limited to: peptides or proteins, (therapeutically active) low molecular weight organic or inorganic compounds (molecular weight less than 5,000), sugars, antigens or antibodies, therapeutic agents already known in the art, antigenic cells, antigenic cellular fragments, cellular fractions, modified, attenuated or de-activated pathogens (e.g. virus, bacteria, fungus, protozoa, plasmodium, or mycobacterium), wherein a pathogen may be attenuated or deactivated chemically, by irradiation, mutation, serial passage, or other known method.

In some embodiments one or more implants may be implanted or administered via one or more route of administration including but not limited to oral, sublingual, transdermal, ophthalmic, parenteral, subcutaneous, intravenous, intramuscular, by inhalation, topical, rectal, nasal, buccal, vaginal, or via an implant.

In some methods of use one or more implants may be implanted or administered via one or more route of administration including but not limited to oral, sublingual, transdermal, ophthalmic, parenteral, subcutaneous, intravenous, intramuscular, by inhalation, topical, rectal, nasal, buccal, vaginal, or via an implant.

In some methods of use one or more RNA stabilizing composition described herein may be used to produce an implant comprising at least one or more RNA substance and at least one or more RNA stabilizing substance. In some methods of use one or more RNA stabilizing composition described herein may be used to produce an implant comprising at least one or more RNA substance, at least one or more RNA stabilizing substance, and at least one or more cellular uptake agent.

In some methods of use one or more RNA substance and one or more RNA stabilizing substance described herein may be combined to produce an implant comprising at least one or more RNA substance and at least one or more RNA stabilizing substance. In some methods of use one or more RNA substance and one or more RNA stabilizing substance, and one or more cellular uptake agent may be combined to produce an implant comprising at least one or more RNA substance, at least one or more RNA stabilizing substance, and at least one or more cellular uptake agent. In other non-limiting example methods of use, one or more additional substance may be combined with one or more RNA substance and one or more RNA stabilizing substance to produce an implant, wherein an implant may also comprise one or more additional substances described herein including, one or more cellular uptake agents, additional RNA stabilizing substances, additional RNA substances, additive substances, inorganic cations (or salt thereof), buffering agents, solvents, or water, as non-limiting examples.

In other methods of use one or more RNA stabilizing composition described herein may be used for one or more of the following applications, including, but not limited to, treating, preventing, or diagnosing a disease or medical indication; producing a vaccine, medicament, or therapeutic agent; or producing a polypeptide or cellular response in one or more of the following organisms or cells, which may include but are not limited to: humans, primates, animals, vertebrate animals, eukaryotic cells, eukaryotes, protozoa, prokaryotic cells, plant cells, plants, fungal cells, fungi, insect cells, insects, bacterial cells, bacteria, mycoplasma, protozoa, plasmodium, or mammalian cells, including but not limited to the cells of primates, animals, vertebrate animals, and the cells of humans.

In some methods of use one or more RNA stabilizing composition described herein may be used for in vivo, in vitro, in situ, or ex vivo applications. Non-limiting applications may include one or more of the following: treating or preventing a disease in an animal; laboratory or diagnostic, including analytical or sequencing applications; improving plant growth or stress tolerance; improving crop yield or nutrition efficiency; treating insects, worms, other pests; protein production, such as antibodies or enzymes; use as an antifungal, antimicrobial, or antiviral; use as a pesticide or herbicide, or combinations thereof.

In other methods of use one or more RNA stabilizing composition described herein may be used for producing, or may be used in conjunction with one or more of the following, including but not limited: syringes, prefilled syringes (including multi-compartment syringes), injection, nasal sprays, transdermal patches, eye drops, oral sprays, aerosols, inhalers, nebulizers, oral tablets, pills, sublingual tablets, sublingual drops, suppositories, mucosal sprays, creams, lozenges, lotions, balms, syrups, ointments, implants, mists, or sprays.

In other methods of use one or more RNA stabilizing composition described herein may be used in a composition that also comprises a cellular uptake agent and used as a mucosal spray. Wherein, a mucosal spray may be any suitable chamber or container that can be squeezed, pressurized, or applied in such a manner to aerosolize, spray, mist, aspirate, drop, squirt, or otherwise apply, administer, or direct a composition comprising at least one RNA stabilizing substance and at least one or more RNA substance, and optionally one or more cellular uptake agent, onto a mucosal surface such as a nasal passage, airway, throat, lung, eye, or other mucosal surface within a human, primate, animal, or vertebrate animal.

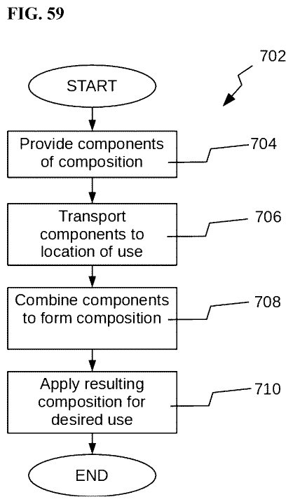

FIG. 59 is a flowchart that summarizes a process 702 for producing and using a stabilized RNA product in accordance with the present disclosure. The process 702 is initiated by providing (704) components of the RNA product or composition. The components may be obtained separately or may be provided as part of a kit or in a preloaded, syringe or multi-compartment syringe as described above, among other possibilities. Moreover, the composition, components, or combinations of components, may be provided in bottles, containers, vials, tubes, syringes, blisters, capsules, cartridges, or other packaging methods. The components may then be stored at the location of use or transported (706) to the location of use. Depending on the specific implementation, the components or some of the components may be refrigerated, frozen, or otherwise maintained in a temperature-controlled environment during transportation and storage.

When ready for use, the components can be combined (708) to yield the desired composition. For example, the individual components may be combined in a bottle, vial, or other container by adding the individual components to the container, e.g., by pouring, using a pipette, syringe, or the like, by adding lyophilized pellets, by measuring powders, or by any other suitable method. In certain implementations, the components may be mixed by breaking a breakable seal of a multi-compartment container such as a multi-compartment syringe. Finally, the resulting composition may be applied (710) for the desired use (such as administering a vaccine, medicament, or therapeutic agent). As otherwise noted herein, the stabilized RNA products of the present disclosure may be utilized in a variety of fields such as pharmaceuticals, therapeutics, diagnostics, or agriculture. In addition, the product may be packaged and distributed as a pharmaceutical composition, biostimulant composition, embedded complex, or implant.

Moreover, in the case of pharmaceutical compositions, the product may be packaged and distributed for administration orally, sublingually, transdermally, ophthalmically, parenterally, subcutaneously, intravenously, intramuscularly, by inhalation, topically, rectally, nasally, buccally, vaginally, or via an implant as otherwise described herein. The RNA product may be used for a variety of applications including treating a disease, preventing a disease, or producing a cellular response as otherwise described herein. The product may be used in in vivo, in vitro, in situ, or ex vivo applications. Non-limiting applications may include: treating or preventing a disease in an animal; laboratory or diagnostic analytical or sequencing applications; improving plant growth or stress tolerance; treating insects or pests; protein production, such as antibodies or enzymes; use as an antifungal, antimicrobial, or antiviral; use as a pesticide or herbicide, or combinations thereof. Accordingly, applying the resulting composition for the desired use will vary depending on the nature of the composition and the intended use among other things.

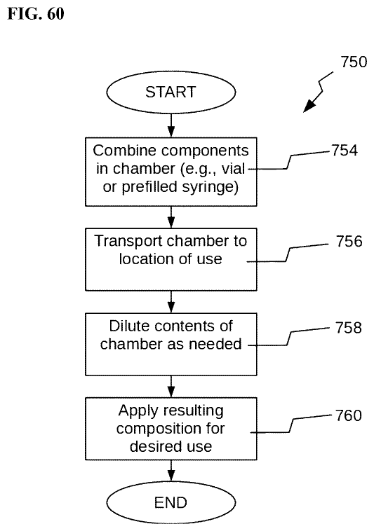

FIG. 60 is a flowchart that summarizes a process 750 for producing and using an RNA product in accordance with the present disclosure. The process 750 is initiated by providing (754) components of the RNA product or composition and combining them in a chamber or combining them and adding the combination to a chamber. The chamber may be any suitable chamber and may be, as non-limiting examples, a single use or multiuse vial. Moreover, the chamber may be, as non-limiting examples, bottles, containers, vials, tubes, syringes (including prefilled or multi-compartment or single use syringes), blisters, capsules, cartridges, or other packaging. The chamber with components may then be stored at the location of use or transported (756) to the location of use. Depending on the specific implementation, the chambers, holding components, may be refrigerated, frozen, or otherwise maintained in a temperature-controlled environment during transportation and storage.

When ready for use, the components in the chamber may be combined with one or more diluents (758) to yield the desired concentration for final use. For example, the chambers produced at step (754) may, for example, contain a concentrated mixture or a composition that has a selectively adjustable viscosity, needing dilution, such as to reduce viscosity, or solids needing to be dissolved, for example in a bottle, vial, syringe, or other container. Alternatively, when ready for use, if the components introduced into the chamber are such that no dilution is needed then the contents are not diluted. Alternatively, part or all of the contents of the chamber may be withdrawn and added to another container, a non-limiting example being a bag containing an IV solution. At this stage other materials may be added.

Finally, the resulting composition may be applied (760) for the desired use (such as administering a vaccine, medicament, or therapeutic agent). As otherwise noted herein, the stabilized RNA products of the present disclosure may be utilized in a variety of fields such as therapeutics, diagnostics, or agriculture. In addition, the product may be packaged and distributed as a pharmaceutical composition, biostimulant composition, embedded complex, or implant.

Moreover, in the case of pharmaceutical compositions, the product may be packaged and distributed for administration orally, sublingually, transdermally, ophthalmically, parenterally, subcutaneously, intravenously, intramuscularly, by inhalation, topically, rectally, nasally, buccally, vaginally, or via an implant as otherwise described herein. The RNA product may be used for a variety of applications including treating a disease, preventing a disease, or producing a cellular response as otherwise described herein. The product may be used in in vivo, in vitro, in situ, or ex vivo applications. Non-limiting applications may include treating or preventing a disease in an animal; laboratory or diagnostic analytical or sequencing applications; improving plant growth or stress tolerance; treating insects or pests; protein production, such as antibodies or enzymes; use as an antifungal, antimicrobial, or antiviral; or use as a pesticide or herbicide, or combinations thereof. Accordingly, applying the resulting composition for the desired use will vary depending on the nature of the composition and the intended use among other things.

As a non-limiting example, a chamber may be at least partially filled with components comprising one or more RNA substance and one or more RNA stabilizing substance followed by the chamber being prepared for shipping and storage (as a non-limiting example by undergoing steps comprising being packaged or placed in a shipping and storage container) followed by the chamber being transported to the location use, then removed from packaging and administered to a patient or subject in need thereof as a vaccine, medicament, or therapeutic agent. Additionally, if necessary, prior to being administered a composition may be prepared for use by adding one or more diluents to the chamber and mixing, then withdrawing the appropriate amount of diluted mixture from the chamber and administering to a patient or subject in need thereof.

### RNA Stabilizing Substance Kits

The inventors have surprisingly discovered that one or more RNA stabilizing substance may be used in a kit, wherein one or more RNA stabilizing substance as described herein may be provided as at least part of a kit to produce a composition comprising one or more RNA stabilizing substance and one or more RNA substance. The inventors have also surprisingly discovered that one or more RNA stabilizing substance may be packaged into a kit wherein one or more RNA stabilizing substance described herein may be provided to produce a composition comprising a combination of one or more RNA stabilizing substance and one or more RNA substance.

In some embodiments a kit may be provided, wherein a kit may comprise at least one or more RNA stabilizing substance. In some embodiments a kit may be provided, wherein a kit may comprise at least one or more RNA stabilizing substance that may be combined with at least one or more RNA substance to produce an RNA stabilizing composition. In some embodiments one or more RNA stabilizing substance may be provided in a kit wherein one or more RNA stabilizing substance may be combined with one or more RNA substance to produce a composition comprising one or more RNA stabilizing substance and one or more RNA substance. In some embodiments one or more RNA stabilizing substance may be provided in a kit wherein one or more RNA stabilizing substance may be provided to produce a composition comprising one or more RNA stabilizing substance and one or more RNA substance.

In some embodiments one or more RNA stabilizing substance may be provided as at least one or more parts of a kit, wherein one or more RNA stabilizing substance may be provided in a chamber (such as a tube or vial with a friction fit snap cap or a screw top cap, sealed or resealable 96-well plate, sealed or resealable 384-well plate, or other suitable container as non-limiting examples). In some embodiments one or more RNA stabilizing substance may be provided as at least one or more parts of a kit, wherein one or more RNA stabilizing substance may be provided in a chamber where a chamber containing one or more RNA stabilizing substance may be provided in a package (such as a walled container as a non-limiting example). As a non-limiting example, an RNA stabilizing substance may be provided as at least one or more parts of a kit, wherein a chamber containing one or more RNA stabilizing substance may be packaged and at least one of stored at a desired location or shipped to a desired location. In some embodiments one or more RNA stabilizing substance may be provided as at least one or more parts of a kit, wherein one or more RNA stabilizing substance may be placed in a chamber prior to, during, or after packaging said chamber into a kit or package. As a non-limiting example, an RNA stabilizing substance may be provided as at least one or more parts of a kit, wherein an RNA stabilizing substance may be placed in a chamber and then the chamber containing the RNA stabilizing substance may be packaged into at least part of a kit.

In some embodiments a kit may comprise one or more RNA stabilizing substance wherein one or more RNA stabilizing substance may be provided as an individual RNA stabilizing substance (such as an individual RNA stabilizing substance within a chamber as a non-limiting example). As a non-limiting example, a kit may comprise multiple individual RNA stabilizing substances wherein each individual RNA stabilizing substance may be provided in its own individual chamber. In some embodiments a kit may comprise up to 5, or up 10, or up to 15, or up to 20, or up to 25, or up to 30, or up to 50, or up to 70, or up to 100, or up to 200, or up to 500 individual RNA stabilizing substances, wherein the RNA stabilizing substances may be in individual chambers or in individual wells of a multi-well reservoir. In some embodiments a kit may comprise at least 1, or at least 2, or at least 3, or at least 4, or at least 5, or at least 6, or at least 7, or at least 8, or at least 10, or at least 15, or at least 20 individual RNA stabilizing substances, wherein the RNA stabilizing substances may be in individual chambers or in individual wells of a multi-well reservoir.

As a non-limiting example, a kit may comprise one or more RNA stabilizing substances selected from one or more category of RNA stabilizing substances described herein. As a non-limiting example, a kit may comprise RNA stabilizing substances from different categories of RNA stabilizing substances selected from up to 2, or up 3, or up to 4, or up to 5, or up to 6, or up to 7, or up to 8, or up to 9, or up to 10, or up to 12, different RNA stabilizing substance categories, wherein the RNA stabilizing substances may be at least part of a kit as individual RNA stabilizing substances or combinations of two or more RNA stabilizing substances. As a non-limiting example, a kit may comprise RNA stabilizing substances from different categories of RNA stabilizing substances selected from at least 2, or at least 3, or at least 4, or at least 5, or at least 6, different RNA stabilizing substance categories, wherein the RNA stabilizing substances may be at least part of a kit as individual RNA stabilizing substances or combinations of two or more RNA stabilizing substances.

In some embodiments a kit may comprise one or more RNA stabilizing substances wherein one or more RNA stabilizing substance may be combined or mixed with one or more additional RNA stabilizing substance. As a non-limiting example, a kit may comprise two or more RNA stabilizing substances wherein two or more RNA stabilizing substance may be provided as a premixed composition comprising a combination of two or more RNA stabilizing substances. As another non-limiting example, a kit may comprise two or more individual RNA stabilizing substances wherein two or more individual RNA stabilizing substances may be provided (such as each RNA stabilizing substance in its own individual chamber as a non-limiting example) wherein one or more individual RNA stabilizing substance may be combined with one or more additional RNA stabilizing substance to produce a composition comprising two or more RNA stabilizing substances.

In some embodiments a kit may comprise one or more premixed composition comprising a combination of two or more RNA stabilizing substances, wherein two or more RNA stabilizing substances may be combined and provided as a combination of two or more RNA stabilizing substances (such as within a chamber to provide a premixed composition comprising two or more RNA stabilizing substances as a non-limiting example). As a non-limiting example, a kit may comprise one or more premixed composition comprising a combination of two or more RNA stabilizing substances, wherein two or more RNA stabilizing substances may be provided as a premixed combination of two or more RNA stabilizing substances.

In some embodiments a kit may comprise up to 5, or up 10, or up to 15, or up to 20, or up to 25, or up to 30, or up to 50, or up to 70, or up to 100, up to 200, or up to 500, or up to 1000 premixed compositions comprising a combination of two or more RNA stabilizing substances. In some embodiments a kit may comprise at least 1, or at least 2, or at least 3, or at least 4, or at least 5, or at least 6, or at least 7, or at least 8, or at least 10, or at least 15, or at least 20 premixed compositions comprising a combination of two or more RNA stabilizing substances. In some embodiments a premixed composition comprising a combination of two or more RNA stabilizing substances may comprise at least 2, or at least 3, or at least 4 RNA stabilizing substances in a premixed composition. In some embodiments a premixed composition comprising a combination of two or more RNA stabilizing substances may comprise up to 2, or up to 3, or up to 4, or up to 5, or up to 6, or up to 8, or up to 10, or up to 20 RNA stabilizing substances in a premixed composition.

In some embodiments a kit may comprise up to 5, or up 10, or up to 15, or up to 20, or up to 25, or up to 30, or up to 50, or up to 70, or up to 100, or up to 200, or up to 500 total RNA stabilizing substances. In some embodiments a kit may comprise at least 1, or at least 2, or at least 3, or at least 4, or at least 5, or at least 6, or at least 7, or at least 8, or at least 10, or at least 15, or at least 20 total RNA stabilizing substances.

In some embodiments a kit may comprise one or more RNA stabilizing substance, wherein one or more RNA stabilizing substance may be provided as a combination or mixture of at least one or more RNA stabilizing substance and one or more of an additional RNA stabilizing substance or auxiliary substance, or combinations thereof. As a non-limiting example, a kit may comprise a premixed combination of one or more RNA stabilizing substance and one or more additional RNA stabilizing substance or one or more auxiliary substance (or combinations thereof) to provide a premixed composition comprising a combination of one or more RNA stabilizing substance and one or more additional RNA stabilizing substance or one or more auxiliary substance, or combinations thereof. As a non-limiting example, a kit may comprise multiple combinations or mixtures of one or more RNA stabilizing substance and one or more additional RNA stabilizing substance or one or more auxiliary substance, or combinations thereof. In some embodiments one or more RNA stabilizing substance or one or more auxiliary substance may be provided as a mixture or combination of one or more RNA stabilizing substance and one or more auxiliary substance (such as a premixed combination of one or more RNA stabilizing substance and one or more auxiliary substance combined in a single chamber as a non-limiting example) or may be provided as one or more individual RNA stabilizing substance and one or more individual auxiliary substance (such as each substance in its own individual chamber as a non-limiting example).

In some embodiments a kit may comprise up to 5, or up 10, or up to 15, or up to 20, or up to 25, or up to 30, or up to 50, or up to 70, or up to 100, or up to 200, or up to 500, or up to 1000 premixed compositions comprising a combination of one or more RNA stabilizing substance and one or more auxiliary substance. In some embodiments a kit may comprise at least 1, or at least 2, or at least 3, or at least 4, or at least 5, or at least 6, or at least 7, or at least 8, or at least 10, or at least 15, or at least 20 premixed compositions comprising a combination of one or more RNA stabilizing substance and one or more auxiliary substance.

In some embodiments a premixed composition comprising a combination of one or more RNA stabilizing substance and one or more auxiliary substance may comprise up to 2, or up to 3, or up to 4, or up to 5, or up to 6, or up to 8, or up to 10, or up to 20 RNA stabilizing substances in a premixed composition. In some embodiments a premixed composition comprising a combination of one or more RNA stabilizing substance and one or more auxiliary substance may comprise up to 2, or up to 3, or up to 4, or up to 5, or up to 6, or up to 8, or up to 10 auxiliary substances in a premixed composition. In some embodiments a kit may comprise up to 2, or up 3, or up to 4, or up to 5, or up to 6, or up to 8, or up to 10, or up to 20, or up to 50, or up to 100, or up to 200 total auxiliary substances.

In some embodiments a kit may comprise at least one or more label on at least one or more chamber. Labels on chambers may include information about the substance or composition within the chamber. As non-limiting examples, labels on chambers may include the composition, individual components or substances, concentration of substances (as non-limiting examples may be molarity, weight, weight by volume, volume by volume, or percent weight by volume, or percent volume by volume), pH, or additional information such as, manufacturing date, expiration date, or instructions for use or mixing. In some embodiments a kit may comprise a label on one or more chamber, wherein a label may be coded such as by an alphanumeric code (as non-limiting examples may be A-Z, or 1-1000, or combinations thereof such as A1, B102, or C301 or date or time codes or designations (such as 2024 Jun. 30 or 20240630-01), or lot or batch numbers, as non-limiting examples).

In some embodiments a kit may comprise one or more premixed compositions, wherein a composition may comprise at least one or more RNA stabilizing substance (such as 1, 2, 3, 4, 5, or 6, or more RNA stabilizing substance as non-limiting examples). In some embodiments a kit may comprise one or more premixed compositions, wherein a composition may comprise one or more RNA stabilizing substance and one or more additional substances described herein (such as one or more additional RNA stabilizing substance or auxiliary substance, as non-limiting examples). In some embodiments a kit may comprise one or more premixed composition, wherein a composition may comprise two or more RNA stabilizing substances (such as 2, 3, 4, 5, or 6, or more RNA stabilizing substances, as non-limiting examples). In some embodiments a kit may comprise between about 1-1000 premixed compositions. As a non-limiting example, a kit may comprise between about 1-1000, or 1-500, or 1-400, or 2-1000, or 2-500, or 2-400, or 5-1000, or 5-500, or 5-400, or 10-1000, or 10-500, or 10-400 premixed compositions. In some embodiments a kit may comprise 1 or more premixed compositions. In some embodiments a kit may comprise more than 1, or more than 2, or more than 3, or more than 4, or more than 5, or more than 6 premixed compositions. In some embodiments a composition provided as at least part of a kit may comprise at least one or more RNA stabilizing substance, wherein a composition may comprise an individual RNA stabilizing substance or multiple RNA stabilizing substances (such as 2 or more, 3 or more, or 4 or more, or 5 or more RNA stabilizing substances mixed together as a non-limiting example). As a non-limiting example, a composition provided as at least part of a kit may comprise at least one or more RNA stabilizing substance and one or more additional substances described herein (such as one or more additional RNA stabilizing substance or auxiliary substance as non-limiting examples).

In some embodiments a kit may comprise one or more individual RNA stabilizing substances. As non-limiting examples, each individual RNA stabilizing substance may be provided in individual chambers or provided in separate wells of a multi-well reservoir (such as a 96-well plate, as non-limiting examples). In some embodiments a kit may comprise one or more individual auxiliary substances as described herein. As non-limiting examples, each individual auxiliary substance may be provided in individual chambers or provided in separate wells of a multi-well reservoir (such as a 96-well plate, as non-limiting examples).

In some embodiments a kit may comprise one or more RNA stabilizing substance wherein one or more RNA stabilizing substance may be provided as a combination or mixture of at least one or more RNA stabilizing substance and one or more additional substances described herein (such as one or more additional RNA stabilizing substance or auxiliary substance, as non-limiting examples). As a non-limiting example, each composition comprising one or more RNA stabilizing substance and one or more additional substance (such as one or more additional RNA stabilizing substance or auxiliary substance, as non-limiting examples) may be provided as premixed compositions in individual chambers or provided as premixed compositions in separate wells of a multi-well reservoir (such as a 96-well plate, as non-limiting examples).

In some embodiments a kit may comprise one or more individual RNA stabilizing substance (such as multiple individual RNA stabilizing substances as a non-limiting example) or premixed combinations of multiple RNA stabilizing substances (such as premixed combinations of two or more RNA stabilizing substances as a non-limiting example). As a non-limiting example, a kit may comprise multiple individual RNA stabilizing substances or multiple premixed combinations of two or more RNA stabilizing substances; or as a non-limiting example, a kit may comprise a combination of both multiple individual RNA stabilizing substances and multiple premixed combinations of two or more RNA stabilizing substances.

In some embodiments a kit may comprise one or more individual auxiliary substance (such as multiple individual auxiliary substances as a non-limiting example) or premixed combinations of multiple auxiliary substances (such as premixed combinations of two or more auxiliary substances as a non-limiting example) or premixed combinations of one or more auxiliary substance and one or more RNA stabilizing substance (such as premixed combinations of one or more auxiliary substance and one or more RNA stabilizing substance as a non-limiting example). As a non-limiting example, a kit may comprise multiple individual auxiliary substances, or premixed combinations of multiple auxiliary substances, or premixed combinations of one or more auxiliary substance and one or more RNA stabilizing substance; or as a non-limiting example, a kit may comprise a combination of either two or all three of the following: multiple individual auxiliary substances, or premixed combinations of multiple auxiliary substances, or premixed combinations of one or more auxiliary substance and one or more RNA stabilizing substance. As another non-limiting example, a combination of one or more RNA stabilizing substance and one or more auxiliary substance may be a composition comprising an RNA stabilizing substance and a buffering agent or inorganic cation (or salt thereof).

In some embodiments a kit may comprise one or more auxiliary substance (such as 1, 2, 3, 4, 5, 6, 7, 8, 9, 10, 15, 20, 30, 40, 50, or 100, or more auxiliary substances, as non-limiting examples). In some embodiments a kit may comprise a combination or mixture of two or more auxiliary substances (such as 2 or more, or 3 or more, or 4 or more, or 5 or more, or 6 or more, or greater, as non-limiting examples). As a non-limiting example, a combination of two or more auxiliary substances may be a composition comprising a cellular uptake agent and a buffering agent.

In some embodiments a kit may comprise one or more individual RNA stabilizing substance or combinations of multiple RNA stabilizing substances (e.g. two or more RNA stabilizing substances as a non-limiting example) wherein at least one or more RNA stabilizing substance may be combined with one or more RNA substance to produce a composition comprising one or more RNA stabilizing substance and one or more RNA substance.

In some embodiments a kit may comprise one or more individual RNA stabilizing substance or combinations of multiple RNA stabilizing substances (e.g. two or more RNA stabilizing substances as a non-limiting example) wherein at least one or more RNA stabilizing substance may be combined with one or more additional substances described herein (such as one or more additional RNA stabilizing substance or auxiliary substance as non-limiting examples) to produce a composition comprising one or more RNA stabilizing substance and one or more additional substance described herein (such as one or more additional RNA stabilizing substance or auxiliary substances as non-limiting examples).

In some embodiments a kit may comprise one or more individual auxiliary substance described herein, wherein one or more auxiliary substance may be combined with one or more RNA stabilizing substance or one or more RNA substance to produce a composition comprising one or more RNA stabilizing substance or one or more RNA substance and one or more auxiliary substance.

As a non-limiting example, a kit may be provided wherein a kit may comprise one or more individual RNA stabilizing substances that may be combined with one or more RNA substance or one or more additional substances described herein (such as one or more additional RNA stabilizing substance or auxiliary substance as non-limiting examples) to produce a composition comprising at least one or more RNA stabilizing substance and one or more RNA substance or one or more additional substance as described herein (such as one or more additional RNA stabilizing substance or auxiliary substance as non-limiting examples).

As a non-limiting example, a kit may be provided wherein a kit may comprise one or more individual RNA stabilizing substance or multiple RNA stabilizing substances (e.g. two or more RNA stabilizing substances as a non-limiting example) that may be combined with one or more RNA substance or one or more additional substances described herein (such as one or more additional RNA stabilizing substance or auxiliary substance as non-limiting examples) to produce a composition comprising at least one or more RNA stabilizing substance and one or more RNA substance or one or more additional substance as described herein (such as one or more additional RNA stabilizing substance or auxiliary substance as non-limiting examples).

In some embodiments a kit may comprise one or more RNA stabilizing substances, wherein an RNA stabilizing substance may be provided as an individual RNA stabilizing substance or combination or mixture of two or more RNA stabilizing substances. In some embodiments a kit may comprise at least 1, or at least 2, or at least 3, or at least 4, or at least 5, or at least 6 RNA stabilizing substances, wherein an RNA stabilizing substance may be provided as an individual RNA stabilizing substance or combination or mixture of two or more RNA stabilizing substances.

As a non-limiting example, a kit may be provided wherein a kit may comprise multiple individual substances described herein (such as one or more RNA stabilizing substance or auxiliary substance, as non-limiting examples) or multiple combinations (e.g. two or more substances) of substances described herein (such as one or more RNA stabilizing substance or auxiliary substance, as non-limiting examples) that may be combined with one or more RNA substance or one or more additional substance (such as one or more additional RNA stabilizing substance or auxiliary substance, as non-limiting examples) or one or more composition described herein (such as a composition comprising one or more RNA stabilizing substance or one or more auxiliary substance, as non-limiting examples) to produce a composition comprising at least one or more RNA stabilizing substance and one or more RNA substance or one or more additional substances described herein (such as one or more additional RNA stabilizing substance or auxiliary substance, as non-limiting examples). In some embodiments a kit may comprise at least 1, or at least 2, or at least 3, or at least 4, or at least 5, or at least 6 substances described herein (such as an RNA stabilizing substance or an auxiliary substance, as non-limiting examples), wherein one or more substance described herein may be provided as an individual substance or a combination or mixture of two or more substances.

In some embodiments one or more RNA stabilizing substance or a composition comprising at least one or more RNA stabilizing substance may be provided as at least one or more parts of a kit, wherein one or more RNA stabilizing substance or a composition comprising at least one or more RNA stabilizing substance may be provided in a chamber (such as a tube or vial with a friction fit snap cap, or with a screw top cap, or sealed or resealable 96-well plate, or sealed or resealable 384-well plate, or other suitable container as non-limiting examples).

In some embodiments a kit may comprise one or more RNA stabilizing substance or a composition comprising at least one or more RNA stabilizing substance in a chamber (such as a vial, tube, 96-well plate, or 384-well plate as non-limiting examples), where a chamber may be hermetically sealed or may have a resealable seal (such as a screw-cap, snap-cap, friction fit cap, or adhesive seal, as non-limiting examples). In some embodiments a kit may comprise one or more RNA stabilizing substance or a composition comprising at least one or more RNA stabilizing substance with instructions for use, such as mixing with one or more RNA substance, or mixing with a diluent (e.g. water or buffer, as non-limiting examples), as non-limiting examples. Non-limiting examples of instructions for use may include information, descriptions, or directions for mixing one or more RNA stabilizing substance or a composition comprising at least one or more RNA stabilizing substance with one or more RNA substance or one or more additional substances (such as one or more auxiliary substance or additional RNA stabilizing substance, as non-limiting examples) or one or more compositions described herein (such as a composition comprising one or more RNA stabilizing substance or one or more auxiliary substance, as non-limiting examples).

In some embodiments, a kit may comprise one or more RNA stabilizing substance or a composition comprising at least one or more RNA stabilizing substance comprising a liquid (such as a solution, or suspension as a non-limiting examples), or as a solid (such as a powder, tablet, or crystals as non-limiting examples), or as a gel (such as a hydrogel as a non-limiting example), or combinations thereof. In some embodiments a kit may comprise one or more composition comprising at least one or more RNA stabilizing substance wherein a composition may be buffered (such as with one or more buffering agent described herein as a non-limiting example). In some embodiments a kit may comprise one or more composition comprising at least one or more RNA stabilizing substance wherein a composition may comprise one or more buffering agents described herein, such as acetate, phosphate, tris, or citrate as non-limiting examples. In some embodiments a kit may comprise one or more RNA stabilizing substance or one or more composition comprising at least one or more RNA stabilizing substance that may have a pH between about 4-9, or between about 5-8, or between about 6-8. In some embodiments one or more RNA stabilizing substance or a composition comprising at least one or more RNA stabilizing substance may be provided with a pH between about 4-9, or between about 5-8, or between about 6-8.

In some embodiments a kit may comprise one or more RNA stabilizing substance or a composition comprising at least one or more RNA stabilizing substance that may be sterilized prior to packaging (such as by autoclave, filter sterilization, steam, heat, radiation, or other methods known in the art as non-limiting examples). In some embodiments a kit may comprise one or more RNA stabilizing substance or a composition comprising at least one or more RNA stabilizing substance that may be sterilized during manufacturing prior to final packaging or may be sterilized after final packaging or may be sterilized at least prior to final packaging or at least after final packaging. Non-limiting examples of sterilization methods that may be used prior to final packaging or during final packaging or after final packaging may comprise one or more of by autoclave, filter sterilization, steam, heat, radiation (non-limiting examples are gamma and e-beam) or other methods known in the art.

In some embodiments one or more substances (such as one or more RNA stabilizing substance or auxiliary substance as non-limiting examples) or a composition comprising one or more substances described herein (such as a composition comprising one or more RNA stabilizing substance or auxiliary substance, as non-limiting examples) may be provided as at least one or more parts of a kit, wherein one or more substance or composition may be provided in a chamber (such as a tube or vial with a friction fit snap cap, or with a screw top cap, or sealed or resealable 96-well plate, or sealed or resealable 384-well plate, or other suitable container as non-limiting examples). In some embodiments a kit may comprise one or more substances or a composition comprising one or more substances described herein (such as one or more RNA stabilizing substance or auxiliary substance as non-limiting examples) in a chamber (such as a vial, tube, 96-well plate, or 384-well plate as non-limiting examples), wherein the chamber may be hermetically sealed or may have a resealable seal (such as a screw-cap, snap-cap, friction fit cap, or adhesive seal as non-limiting examples).

In some embodiments a kit may comprise one or more substances (such as one or more RNA stabilizing substance or auxiliary substance as non-limiting examples) or a composition comprising one or more substances described herein (such as a composition comprising one or more RNA stabilizing substance or auxiliary substance as non-limiting examples) with instructions for use, such as mixing with one or more RNA substance, or mixing with a diluent comprising water (e.g. water or buffer) as non-limiting examples.

In other non-limiting embodiments, a kit may comprise one or more substances (such as one or more RNA stabilizing substance or auxiliary substance as non-limiting examples) or a composition comprising one or more substances described herein (such as a composition comprising one or more RNA stabilizing substance or auxiliary substance as non-limiting examples) comprising a liquid (such as a solution or suspension as a non-limiting examples), or as a solid (such as a powder, tablet, or crystals as non-limiting examples), or as a gel comprising water (such as a hydrogel as a non-limiting example), or combinations thereof.

In some embodiments a kit may comprise one or more substances (such as one or more RNA stabilizing substance or auxiliary substance as non-limiting examples) or a composition comprising one or more substances described herein (such as a composition comprising one or more RNA stabilizing substance or auxiliary substance, as non-limiting examples) wherein one or more substances or compositions may be buffered (such as with one or more buffering agent described herein as a non-limiting example). As non-limiting examples, a kit may comprise one or more substance (such as one or more RNA stabilizing substance or auxiliary substance as non-limiting examples) or composition described herein (such as a composition comprising one or more RNA stabilizing substance or auxiliary substance as non-limiting examples), wherein a substance or composition may comprise one or more buffering agents such as acetate, phosphate, tris, or citrate as non-limiting examples. In other non-limiting embodiments, a kit may comprise one or more substances (such as one or more RNA stabilizing substance or auxiliary substance, as non-limiting examples) or a composition comprising one or more substances described herein (such as a composition comprising one or more RNA stabilizing substance or auxiliary substance as non-limiting examples) that may have a pH between about 4-9, or between about 5-8, or between about 6-8. In some embodiments one or more substances (such as one or more RNA stabilizing substance or auxiliary substance, as non-limiting examples) or a composition comprising one or more substances described herein (such as a composition comprising one or more RNA stabilizing substance or auxiliary substance, as non-limiting examples) may be provided with a pH between about 4-9, or between about 5-8, or between about 6-8.

In some embodiments a kit may comprise one or more substances (such as one or more RNA stabilizing substance or auxiliary substance, as non-limiting examples) or a composition comprising one or more substances described herein (such as a composition comprising one or more RNA stabilizing substance or auxiliary substance, as non-limiting examples) that may be sterilized prior to packaging (such as by autoclave, filter sterilization, steam, heat, radiation, or other methods known in the art as non-limiting examples). In some embodiments a kit may comprise one or more substances (such as one or more RNA stabilizing substance or auxiliary substance as non-limiting examples) or a composition comprising one or more substances described herein (such as a composition comprising one or more RNA stabilizing substance or auxiliary substance as non-limiting examples) that may be sterilized during manufacturing prior to final packaging or may be sterilized after final packaging or may be sterilized at least prior to final packaging or at least after final packaging.

In some embodiments a kit may comprise one or more substance (such as an RNA stabilizing substance or auxiliary substance, as non-limiting examples) or composition described herein (such as a composition comprising one or more RNA stabilizing substance or auxiliary substance, as non-limiting examples), wherein one or more substance or composition may have a total volume of at least 1 μL, or at least 5 μL, or at least 10 μL, or at least 20 μL, or at least 50 μL, or at least 100 μL, as non-limiting examples. In some embodiments a kit may comprise one or more substance (such as an RNA stabilizing substance or auxiliary substance, as non-limiting examples) or composition described herein (such as a composition comprising one or more RNA stabilizing substance or auxiliary substance, as non-limiting examples), wherein one or more substance or composition may have a total volume of up to 100 L, or up to 10 L, or up to 1 L, or up to 100 mL, or up to 50 mL, or up to 20 mL, or up to 10 mL, or up to 5 mL, or up to 2 mL, or up to 1 mL, as non-limiting examples.

In some embodiments a kit may comprise one or more substance (such as an RNA stabilizing substance or auxiliary substance, as non-limiting examples) or composition described herein (such as a composition comprising one or more RNA stabilizing substance or auxiliary substance, as non-limiting examples), wherein at least one substance or composition in a kit may have an endotoxin level less than 100 endotoxin units/mL; or less than 50 endotoxin units/mL; or less than 20 endotoxin units/mL; or less than 10 endotoxin units/mL; or less than 5 endotoxin units/mL; or less than 2 endotoxin units/mL; or less than 1 endotoxin units/mL. One of ordinary skill in the art would appreciate that endotoxin testing is known art, as a non-limiting example, endotoxin level may be measured using a limulus amebocyte lysate (LAL) assay, or other suitable method.

In some embodiments a kit may comprise one or more RNA stabilizing substance or one or more premixed composition comprising one or more RNA stabilizing substance, wherein one or more RNA stabilizing substance may be provided in a chamber, wherein the total weight of all RNA stabilizing substances in a chamber may be at least Ing, or at least 1 μg, or at least 10 μg, or at least 100 μg, or at least 1 mg, or at least 2 mg, or at least 5 mg, or at least 10 mg, or at least 20 mg, or at least 50 mg, or at least 100 mg, or at least 200 mg, or at least 500 mg, as non-limiting examples.

In some embodiments a kit may comprise one or more RNA stabilizing substance or one or more premixed composition comprising one or more RNA stabilizing substance, wherein one or more RNA stabilizing substance may be provided in a chamber, wherein the total weight of all RNA stabilizing substances in a chamber may be less than 1 kg, or less than 100 g, or less than 10 g, or less than 5 g, or less than 1 g, or less than 500 mg, or less than 200 mg, as non-limiting examples.

In some embodiments a kit may comprise one or more RNA stabilizing substance or one or more premixed composition comprising one or more RNA stabilizing substance, wherein the concentration of at least one or more RNA stabilizing substance in a kit may be at least 1 nM, or at least 1 μM, or at least 100 μM, or at least 1 mM, or at least 5 mM, or at least 10 mM, or at least 20 mM, or at least 50 mM, or at least 100 mM, or at least 200 mM, or at least 500 mM, or at least 1M, as non-limiting examples. In some embodiments a kit may comprise one or more RNA stabilizing substance or one or more premixed composition comprising one or more RNA stabilizing substance, wherein the concentration of at least one or more RNA stabilizing substance in a kit may be less than 10M, or less than 5M, or less than 4M, or less than 2M, or less than 1M, or less than 500 mM, as non-limiting examples.

In some embodiments a kit may comprise one or more auxiliary components, as non-limiting examples an auxiliary component may comprise one or more auxiliary substances (non-limiting examples of an auxiliary substance may include one or more cellular uptake agents, additive substances, inorganic cations (or salts thereof), RNA substances (e.g. a control RNA as a non-limiting example), activator substances, dissolution substances, buffering agents, water, solvents, diluents, or combinations thereof, as non-limiting examples), or one or more empty chambers (such as one or more empty tube, 96-well plate, or vial as non-limiting examples) that may be used for mixing or storage, or prefilled or empty syringes, or spin columns (such as desalting, buffer exchange, or size exclusion columns, or nucleic acid purification columns such as silica or silica gel columns, as non-limiting examples), or filters (a non-limiting example of a filter may be a sterile or non-sterile filter, such as an about 0.22 μm filter, or about 0.45 μm filter, or filter between about 0.1-1 μm as non-limiting examples), or one or more additional enzymes or reagents (such as a reverse-transcriptase enzyme and associated buffers and nucleotides, or an RNA loading dye or other materials to prepare compositions for analysis by gel electrophoresis, as non-limiting examples), or combinations thereof.

As a non-limiting example, an auxiliary component comprising one or more empty chambers, may comprise an empty chamber with a volume between about 0.1 mL-100 mL, or between about 0.1 mL-50 mL, or between about 0.1 mL-25 mL, or between about 0.1 mL-10 mL, or between about 0.1 mL-5 mL, or between about 0.1 mL-2.5 mL, or between about 0.1-2 mL, or between about 0.1 mL-1.5 mL, or between about 0.1 mL-1 mL, or between about 0.1 mL-0.6 mL. As a non-limiting example, an auxiliary component comprising one or more empty chambers, may comprise an empty chamber with a volume of less than about 100 mL, or less than about 50 mL, or less than about 25 mL, or less than about 10 mL, or less than about 5 mL, or less than about 2.5 mL, or less than about 2 mL, or less than about 1.5 mL, or less than about 1 mL, or less than about 0.6 mL.

In other non-limiting embodiments, a kit may comprise one or more auxiliary components wherein an auxiliary component may comprise a control RNA such as an RNA encoding a fluorescent or bioluminescent protein (such as green fluorescent protein (GFP), enhanced GFP (eGFP), red fluorescent protein (RFP), mCherry, or firefly luciferase as non-limiting examples).

In some embodiments a kit may comprise instructions for use, such as instructions for mixing, diluting, or combining one or more substances or compositions provided. In some embodiments a kit may comprise written instructions comprising one or more sheets of material separate from the packaging, or may comprise sheets of material inserted into or attached to packaging, (non-limiting examples of sheets of material may be one or more sheets comprising paper or plastic with at least one sheet comprising information as instructions comprising words or illustrations). In some embodiments a kit may comprise written instructions comprising material that may be at least part of the packaging (non-limiting examples may be one or more parts of packaging comprising paper or plastic with at least part of the packaging comprising information as instructions comprising words or illustrations). In some embodiments a kit may comprise instructions comprising one or more digital storage device (non-limiting examples may be optical storage media (non-limiting examples are CD or other media read using at least in part a laser or other optical source) or printed storage media encoded with machine readable information, magnetic storage media, electronic memory media or electronic storage media (a non-limiting example of electronic storage media is flash drives)), comprising information that may be displayed to users as instructions comprising words or illustrations or video or audio. In some embodiments a kit may comprise instructions to obtain information that may be displayed to users as instructions comprising words or illustrations (non-limiting examples of instructions to obtain instruction information may be QR codes or URL addresses for words, illustrations, or video or audio).

In some embodiments a kit may comprise instructions for use, such as instructions for mixing, diluting, or combining one or more substances or compositions provided. As non-limiting examples a kit may comprise instructions that comprise a method of use comprising a sequence of actions that may be used for at least one of mixing, diluting, or combining, one or more component or composition; or holding or storing a component or composition at a specified temperature for specified duration or otherwise using at least one component of the kit. In some embodiments a kit may comprise instructions that describe at least one method of use of the kit. In some embodiments a kit may comprise instructions that describe obtaining instructions (as a non-limiting example, describing obtaining instructions by downloading instructions from a URL) that describe at least one method of use of the kit. In some embodiments a kit may comprise packaged materials and on-line instructions coincident (as a non-limiting example, coincident instructions are instructions adjacent to or directly linked to the description of the kit) or coincident on-line instructions that describe obtaining instructions (as a non-limiting example, describing obtaining instructions by downloading instructions from a URL) that describe at least one method of use of the kit. As a non-limiting example, instructions may provide information, directions, or steps for mixing or storing one or more RNA stabilizing substance with one or more RNA substance. As another non-limiting example, instructions may provide information, directions, or steps for combining at least one or more RNA stabilizing substance or one or more composition comprising at least one or more RNA stabilizing substance with at least one or more RNA substance.

In other non-limiting embodiments, a kit may comprise instructions for use that may provide instructions for mixing one or more component or combinations of components with one or more RNA substance or RNA stabilizing substance to produce a composition comprising at least one or more RNA substance and at least one or more RNA stabilizing substance. As a non-limiting example, instructions may also provide information, directions, or steps for diluting, mixing, or storing (such as volumes, concentrations, pH, incubation time or temperature, storage conditions, or storage time or temperature as non-limiting examples) one or more composition comprising at least one or more RNA substance and one or more RNA stabilizing substance or additional kit components (such one or more auxiliary substance as a non-limiting example). As a non-limiting example, a kit may comprise instructions for storing a composition comprising at least one or more RNA substance and one or more RNA stabilizing substance or additional kit components (such as one or more auxiliary substance as a non-limiting example) at or above freezing (such as at about −80° C. or greater, or at about −60° C. or greater, or at about −40° C. or greater, or at about −20° C. or greater, or at about −10° C. or greater, or at about 0° C. or greater, or at about 4° C. or greater, or at about 10° C. or greater, or at about 20° C. or greater, or at about 30° or greater, or at about 40 C.° or greater, or at about 50 C.° or greater).

As non-limiting examples, a kit may comprise instructions for use for performing accelerated stability testing or screening using one or more composition comprising a combination of one or more RNA substance and one or more RNA stabilizing substance or additional kit components (such as one or more auxiliary substance as a non-limiting example), such as mixing one or more RNA substance with one or more RNA stabilizing substance and storing the combination at one or more specified temperature for one or more specified period of time, as a non-limiting example. As non-limiting examples, instructions for accelerated stability testing or screening one or more RNA stabilizing composition may comprise specified storage conditions at one or more temperature between about 20-70° C., or between about 20-60° C., or between about 30-70° C., or between about 30-60° C., or between about 40-70° C., or between about 40-60° C., or between about 50-70° C. As non-limiting examples, instructions for accelerated stability testing or screening one or more RNA stabilizing composition may comprise specified storage conditions at one or more temperature of about 20° C. or greater, or about 25° C. or greater, or about 30° C. or greater, or about 35° C. or greater, or about 40° C. or greater, or about 45° C. or greater, or about 50° C. or greater. As non-limiting examples, instructions for accelerated stability testing or screening one or more RNA stabilizing composition may comprise specified storage conditions for one or more specified period of time, such as about 4 hours (hrs) or longer, or about 6 hrs or longer, or about 8 hrs or longer, or about 12 hrs or longer, or about 16 hrs or longer, or about 24 hrs or longer, or about 36 hrs or longer, or about 48 hrs or longer, or about 72 hrs or longer, or about 96 hrs or longer, or about 1 week or longer, or about 2 weeks or longer, or about 4 weeks or longer, as non-limiting examples.

In other non-limiting embodiments, a kit may comprise instructions for use for mixing (or otherwise combining) or storing a composition comprising one or more RNA substance and one or more RNA stabilizing substance or one or more additional kit components (such one or more auxiliary substance as a non-limiting example). As a non-limiting example, instructions may comprise specified volumes, ratios, or concentrations for mixing or otherwise combining one or more auxiliary substance with one or more RNA substance and one or more RNA stabilizing substance, wherein an auxiliary substance may be combined with one or more RNA substance either prior to or after an RNA substance has been combined with one or more RNA stabilizing substance. As a non-limiting example, a kit may comprise instructions for combining one or more cellular uptake agent described herein (such as a lipid or ionizable lipid) with one or more RNA substance at specified ratio, volume, or concentration, either prior to or following combining an RNA substance with one or more RNA stabilizing substance. As another non-limiting example, a kit may comprise instructions for combining one or more buffering agent (such as to lower or raise the pH as a non-limiting example) or one or more inorganic cation (or salt thereof) (such as to increase the ionic strength as a non-limiting example) at a specified pH, ratio, volume, or concentration (such as one or more pH, ratio, or concentration described herein as a non-limiting example) with one or more RNA stabilizing composition. As another non-limiting example, a kit may comprise instructions for diluting or adjusting the pH (e.g. lower or raise the pH) of one or more RNA stabilizing composition to a specified pH, ratio, volume, or concentration using one or more diluent comprising water (such as a buffering agent or water). As a non-limiting example, a kit may comprise instructions for mixing (or otherwise combining) an RNA stabilizing composition with one or more buffering agent or inorganic cation (or salt thereof), prior to storage. As another non-limiting example, a kit may comprise instructions for mixing (or otherwise combining) an RNA stabilizing composition with one or more buffering agent or suitable diluent (e.g. water, buffer, or other suitable diluent, as non-limiting examples) following storage, with instructions for diluting or adjusting the pH of an RNA stabilizing composition using one or more buffering agent or suitable diluent.

As a non-limiting example, instructions for adjusting the pH of one or more RNA stabilizing composition may comprise instructions for adding an activator substance or buffer that may adjust the pH below a specified value prior to storage or stability testing, such as instructions for adjusting the pH below about 8, or to below about 7, or to below about 6. Instructions for adjusting the pH of one or more RNA stabilizing composition may also comprise instructions for adding a dissolution substance or buffer that may adjust the pH above a specified value following storage or prior to use or analysis, such as instructions for adjusting the pH to above about 5, to above about 6, or to above about 7. As a non-limiting example, instructions for adjusting the pH of one or more RNA stabilizing composition may comprise instructions for adjusting the pH to about 5-9, or to about 5-8, or to about 5-7, or to about 5-6, or to about 6-9, or to about 7-9, or to about 6-8, or to about 7-8. As a non-limiting example, instructions for diluting one or more RNA stabilizing composition may comprise instructions for diluting a composition by about 1.5 fold, or by about 2 fold, or by about 2.5 fold, or by about 3 fold, or by about 4 fold. As a non-limiting example, diluting a composition 2 fold would be a 2× dilution (e.g. diluting a composition from 1 mL to 2 mL as a non-limiting example).

As a non-limiting example, a kit may comprise instructions for use for mixing (or otherwise combining) an activator substance with one or more RNA stabilizing composition (such as a composition with a selectively adjustable viscosity) that may increase the viscosity of the composition above a specified value or change one or more physical properties of the composition, such as changing the composition from a low viscosity liquid to a gel or viscous fluid comprising water (e.g. a thixotropic fluid) prior to storage or stability testing. An activator substance may adjust the pH to a specified value or range, or increase the ionic strength (such as by increasing the concentration of an inorganic cation) of a composition. As a non-limiting example, a kit may also comprise instructions for use for mixing (or otherwise combining) a dissolution substance with one or more RNA stabilizing composition (such as a composition with a selectively adjustable viscosity) that may decrease the viscosity of the composition below a specified value or change one or more physical properties of the composition, such as changing the composition from a gel or viscous fluid comprising water (e.g. a thixotropic fluid) to a low viscosity liquid following storage or prior to use or analysis. A dissolution substance may adjust the pH to a specified value or range, or decrease the ionic strength (such as by diluting or reducing the concentration of an inorganic cation), or reduce the concentration of one or more RNA stabilizing substance of a composition.

As a non-limiting example, an activator substance may comprise one or more buffering agent described herein (such as tris, acetate, citrate, or phosphate as non-limiting examples), or one or more inorganic cation or salt thereof comprising an inorganic cation as described herein, (such as Li, Na, K, Mg, Mn, Au, Ag, as non-limiting examples). As a non-limiting example, a dissolution substance may comprise one or more buffering agent described herein (such as tris, acetate, citrate, or phosphate as non-limiting examples) or water or other suitable solution or solvent for diluting or reducing the viscosity of a composition described herein.

In other non-limiting embodiments, a kit may comprise instructions for use for measuring or analyzing RNA stability following storage of one or more RNA stabilizing composition. As a non-limiting example, a kit may comprise instructions for measuring or analyzing RNA stability such as by performing gel electrophoresis, size exclusion chromatography, using a bioanalyzer, performing reverse transcription, cellular expression (e.g. protein expression of an mRNA, as a non-limiting example), fluorescent assay, or other suitable method, or combinations thereof as non-limiting examples. As a non-limiting example, a kit may comprise instructions for reducing the concentration or removing one or more RNA stabilizing substance or auxiliary substance such as via dialysis, RNA precipitation, using a column or spin column (e.g. silica or silica gel, or desalting, buffer exchange, or size exclusion as non-limiting examples), or dilution, or combinations thereof as non-limiting examples.

As a non-limiting example, a kit may comprise instructions for performing reverse transcription using a reverse transcriptase enzyme following storage of one or more RNA stabilizing composition to help analyze RNA integrity or stability (such as by performing reverse transcription of an RNA following storage and then analyzing the reverse-transcribed material by gel electrophoresis or other suitable method, as a non-limiting example).

As a non-limiting example, a kit may comprise instructions for analyzing or comparing RNA stability of one or more control RNA such as an RNA encoding a fluorescent or bioluminescent protein (such as green fluorescent protein (GFP), enhanced GFP (eGFP), red fluorescent protein (RFP), mCherry, or firefly luciferase, as non-limiting examples). As a non-limiting example, a kit may comprise instructions for mixing (or otherwise combining) or storing a composition comprising one or more control RNA and one or more RNA stabilizing substance or one or more additional kit component (such as one or more auxiliary substance as a non-limiting example). As a non-limiting example, instructions may comprise specified volumes, ratios, or concentrations for mixing or otherwise combining one or more control RNA and one or more RNA stabilizing substance to produce one or more RNA stabilizing compositions described herein. As a non-limiting example, a kit may comprise instructions for storing one or more RNA stabilizing composition comprising a control RNA (such as for accelerated stability testing or screening as a non-limiting example) and analyzing or measuring the stability or integrity of the control RNA or comparing the stability or integrity of the control RNA to one or more additional RNA substance.

In some embodiments a kit may be provided wherein a kit may be transported, shipped, or stored. As non-limiting examples, a kit may be transported or shipped by airplane, automobile (such as truck, van, or car as non-limiting examples) or boat. In some embodiments a kit may be provided wherein a kit may be transported, shipped, or stored at about 20° C. or greater. In some embodiments a kit may be provided wherein a kit may be transported, shipped, or stored at about −80° C. or greater, or at about −60° C. or greater, at about −40° C. or greater, or at about −20° C. or greater, or about −10° C. or greater, or about 0° C. or greater, or about 4° C. or greater, or about 10° C. or greater, or about 20° C. or greater.

In some embodiments one or more RNA stabilizing substance or auxiliary substance may be provided as at least one or more part of a kit wherein the kit may comprise a package or walled container. In some embodiments, a package or walled container may comprise one or more label. As non-limiting examples a label may comprise information about the contents of a kit or one or more kit components, or instructions of use for one or more kit components, or one or more potential use of the kit (such as RNA stability testing or screening, accelerated RNA stability screening, or RNA stability and lipid compatibility screening, as non-limiting examples), or relevant manufacturing or storage information such as lot numbers, trace codes or numbers, expiration dates, storage conditions (e.g. storage temperature), manufacturing date, manufacturer or manufacturing location, or company designs or logos; or combinations thereof, that may provide relevant information about the kit source, storage conditions, or implementation of the kit to the user.

### Non-Limiting Kit Configurations

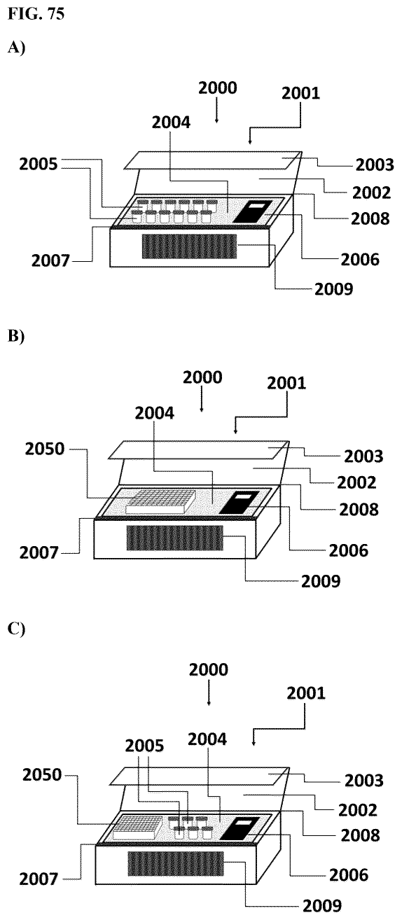

A non-limiting example of a kit comprising one or more RNA stabilizing substance is shown in FIG. 75A. As shown, Kit 2000, may comprise a package 2001 (such as a walled container, as a non-limiting example). Package 2001 may comprise a moveable cover 2002 (such as a lid, top, or sealable portion that allows access to the items contained in package 2001). Moveable cover 2002 may comprise a flap or tab 2003 that fits into slot 2007 to at least partially seal package 2001. Additionally, package 2001 may comprise hinge 2008 to help facilitate movement and opening, closing, or sealing of moveable cover 2002. Package 2001 may comprise supporting material 2004 to at least partially support or hold chamber 2005, wherein supporting material 2004 may be made of cardboard, foam, or other suitable material to at least partially support or hold (e.g. support or restrict movement or reduce force or reduce magnitude of vibrations, as non-limiting examples) one or more components of kit 2000 during shipping or storage. Kit 2000 may comprise one or more RNA stabilizing substance or combinations or mixtures of two or more RNA stabilizing substances in chamber 2005. Chamber 2005 may be one or more type of chamber described herein (such as a tube, vial, syringe, or other suitable container as non-limiting examples) and may be made of glass, plastic, metal or other suitable material described herein as non-limiting examples. Chamber 2005 may also comprise a moveable cover that may be sealed or resealed (such as screw-cap, friction-fit cap, or plug with an access-port as non-limiting examples). Kit 2000 may also comprise insert 2006 with instructions for use, such as describing how to mix, store, or dilute one or more components of kit 2000 as non-limiting examples. Kit 2000 may also comprise label 2009 affixed to package 2001, wherein label 2009 may include information about kit 2000, such as what components are included in kit 2000, expiration date, or other pertinent information as non-limiting examples. As a non-limiting example, package 2001 containing one or more kit components may be at least partially sealed (such as by an adhesive seal, tape, mechanical interference, latch, or tab, or other suitable seal, as non-limiting examples), such as to at least partially prevent one or more kit components from spilling or falling out of the package during transportation or storage, as a non-limiting example.

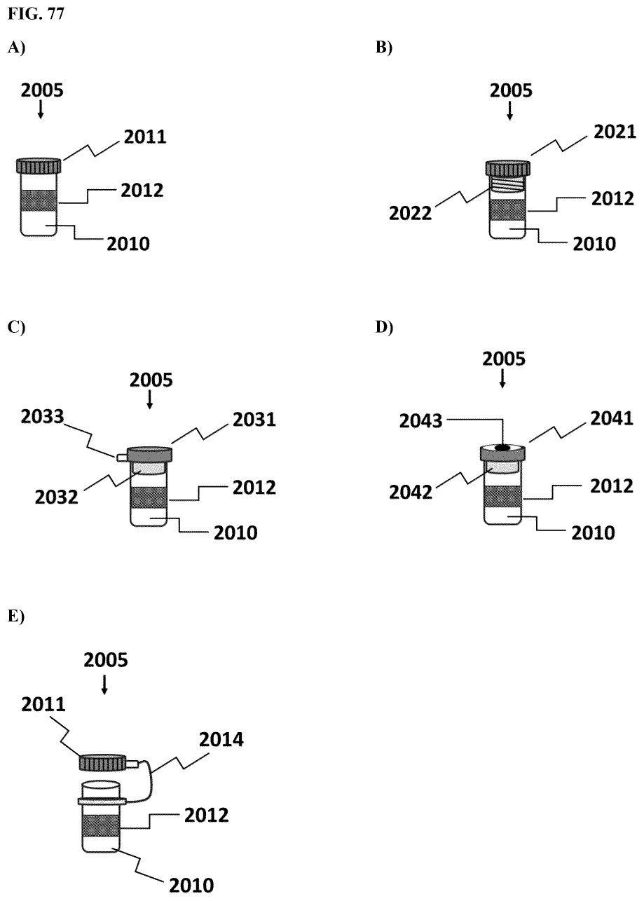

In some embodiments, chamber 2005 may comprise a chamber as shown in FIG. 77A wherein the chamber may be made of plastic (such as polystyrene, polypropylene, or polyethylene, as non-limiting examples), glass, metal, or other polymeric or suitable material, as non-limiting examples. Chamber 2005 may comprise a reservoir 2010 (such as a tube or vial) and a moveable cover 2011 (such as a sealable or resealable cap, top, or plug wherein the cap, top, or plug may be a screw-cap or friction-fit cap or plug as non-limiting examples). Chamber 2005 may also comprise a label 2012 affixed to chamber 2005 with information about the material inside chamber 2005 (e.g. type of RNA stabilizing substance or composition, concentration, weight, volume, pH, or alphanumeric code, as non-limiting examples). Non-limiting examples of chamber 2005 are shown in FIG. 77B-E. FIG. 77B shows a non-limiting example of chamber 2005 comprising a reservoir 2010 with a resealable screw-cap 2021 with threads 2022 and label 2012. FIG. 77C shows a non-limiting example of chamber 2005 with label 2012 and reservoir 2010, wherein reservoir 2010 may have an attached sealable or resealable friction-fit cap 2031 with friction fit insert 2032 and hinge 2033, such that friction-fit cap 2031 may be attached to reservoir 2010 by hinge 2033 and seals reservoir 2010 with friction-fit insert 2032. FIG. 77D shows a non-limiting example of chamber 2005 with label 2012 and reservoir 2010, wherein reservoir 2010 may have a sealable or resealable friction-fit cap 2041 and friction fit insert 2042 and access port 2043. Access port 2043 may at least partially allow access to the contents inside of reservoir 2010 such as by puncturing access port 2043 (such as by a syringe, needle, pipette tip, or other suitable hollow tube, as non-limiting examples). In some other non-limiting embodiments, access port 2043 may comprise, rubber, plastic, cork, foam, or other suitable material that allows the port to be punctured or punctured and at least partially self-seal upon removal of a needle or hollow tube from the access port. In some embodiments friction-fit cap 2041 may be sealed with a band (not shown) or adhesive (not shown) to at least partially secure cap 2041 to reservoir 2010. As a non-limiting example, the band that may be used to at least partially secure cap 2041 to reservoir 2010 may be made of metal, plastic, heat-shrink tubing, or other suitable material that may be crimped or sealed around cap 2041 or reservoir 2010.

In some embodiments, chamber 2005 may comprise a chamber as shown in FIG. 77E wherein a chamber may comprise a reservoir 2010, a label 2012, and a moveable cover 2011 (such as a sealable or resealable cap, top, or plug wherein the cap, top, or plug may be a screw-cap or friction-fit cap or plug as non-limiting examples), wherein the moveable cover may be attached or otherwise secured to the reservoir by securing attachment 2014. Securing attachment 2014 may comprise a loop, band, or ring or other suitable attachment, that may be attached or secured to moveable cover 2011 and wrapped around or otherwise attached or secured to reservoir 2010, such as to secure the moveable cover (e.g. a screw-cap, friction fit cap, or plug, as non-limiting examples) to the reservoir during opening, closing, or other use of chamber 2005, as non-limiting examples.

A non-limiting example of a kit comprising one or more RNA stabilizing substance is shown in FIG. 75B. As shown, Kit 2000, may comprise a package 2001 (such as a walled container, as a non-limiting example). As previously described, package 2001 may comprise a moveable cover 2002, hinge 2008, or optional flap or tab 2003 that fits into optional slot 2007 to seal package 2001. Package 2001 may comprise supporting material 2004 to at least partially support or hold chamber 2050, wherein supporting material 2004 may be made of cardboard, foam, plastic, paper, or other suitable material to at least partially support or hold (e.g. support or restrict movement or reduce force or reduce magnitude of vibrations as non-limiting examples) one or more components of kit 2000 during shipping or storage. Kit 2000 may comprise one or more RNA stabilizing substance or combinations or mixtures of two or more RNA stabilizing substances in chamber 2050. Chamber 2050 may be a chamber with one or more individual reservoirs or a reservoir with multiple wells (herein referred to as a multi-well reservoir) (such as a plate or tray, e.g. a 96-well plate or 384-well plate, as non-limiting examples) and may be made of plastic (such as polystyrene, polypropylene, or polyethylene, as non-limiting examples), glass, metal, or other polymeric or suitable material, as non-limiting examples. Chamber 2050 may also comprise a moveable cover (not shown) that may be sealed or resealed (such as an adhesive cover, sliding lid, cap, or cover that lays over the top of one or more reservoir or one or more wells of a multi-well reservoir as non-limiting examples). Kit 2000 may also comprise insert 2006 with instructions for use, such as describing how to mix, store, or dilute one or more components of Kit 2000 as non-limiting examples. Kit 2000 may also comprise label 2009 affixed to package 2001, wherein label 2009 may include information about kit 2000, such as what components are included in kit 2000, expiration date, or other pertinent information as non-limiting examples.

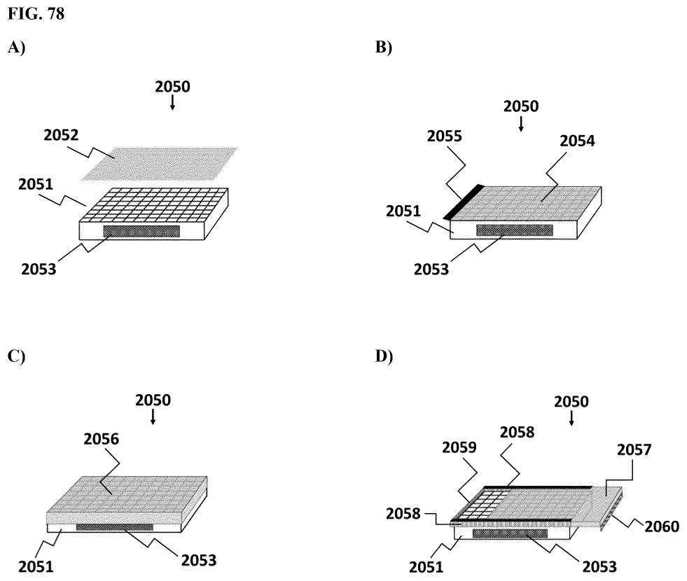

In some embodiments, chamber 2050 may comprise a chamber as shown in FIG. 78A wherein a chamber may comprise a multi-well reservoir 2051 (such as a tray or plate, e.g. a 96-well plate, or 384-well plate, as non-limiting examples) made of plastic (e.g. polypropylene, polystyrene, or polyethylene, as non-limiting examples), glass, or metal, as non-limiting examples, and a moveable cover 2052 (such as a sheet with adhesive seal, sliding lid, or cover that fits over the top of at least one or more wells of the reservoir, as non-limiting examples) that at least partially covers or at least partially seals one or more wells of reservoir 2051. Chamber 2050 may also comprise a label 2053 affixed to the chamber with information about the material inside of the chamber (e.g. type of RNA stabilizing substances or compositions, concentration, weight, volume, pH, or alphanumeric code as non-limiting examples). Non-limiting examples of chamber 2050 are shown in FIG. 78B-D. FIG. 78B shows a non-limiting example of chamber 2050 wherein a chamber may comprise a multi-well reservoir 2051 with a label 2053, a seal 2054 and optional tab 2055, wherein seal 2054 may comprise an adhesive on at least part of the seal (e.g. on one side or both sides of the seal, or on one or more edges of the seal, as non-limiting examples) such that seal 2054 may be at least partially secured to reservoir 2051 to at least partially cover or at least partially seal one or more wells of reservoir 2051. In some embodiments seal 2054 may also be at least partially removed or optionally at least partially resecured over reservoir 2051 to at least partially access some of the contents of reservoir 2051. In some embodiments seal 2054 may optionally be punctured (such as by a syringe, needle, pipette tip, or other suitable hollow tube as non-limiting examples) to at least partially access some of the contents inside reservoir 2051. Seal 2054 may be an adhesive sheet made of foil, plastic, metal, glass, polymeric material, or other suitable material as non-limiting examples. FIG. 78C shows a non-limiting example of chamber 2050 wherein a chamber may comprise a multi-well reservoir 2051, a label 2053, and a moveable cover 2056, wherein moveable cover 2056 fits over the top of reservoir 2051 to at least partially cover or at least partially seal one or more wells of reservoir 2051. Moveable cover 2056 may be lifted or moved to at least partially access some of the contents of reservoir 2051. Moveable cover 2056 may be made of plastic, metal, glass, or other polymeric or suitable material. FIG. 78D shows a non-limiting example of chamber 2050 wherein a chamber may comprise multi-well reservoir 2051, a moveable cover 2057, and a label 2053, wherein moveable cover 2057 may slide into one or more grooves 2058 to at least partially cover or at least partially seal one or more wells of reservoir 2051. Additionally, chamber 2050 may comprise backstop 2059 to help align moveable cover 2057 to slide over reservoir 2051. Moveable cover 2057, may also comprise lip 2060 to help align moveable cover 2057 to slide over reservoir 2051. Moveable cover 2057 may slide or move to at least partially access some of the contents of reservoir 2051. Moveable cover 2057 may be made of plastic, metal, glass, or other polymeric or suitable material.

As a non-limiting example, a multi-well reservoir may comprise at least 2 or more wells, or at least 4 or more wells, or at least 8 or more wells, or at least 12 or more wells, or at least 24 or more wells, or at least 48 or more wells, or at least 96 or more wells. As another non-limiting example, a multi-well reservoir may comprise up to about 1000 wells, or up to about 500 wells, or up to about 400 wells, or up to about 200 wells, or up to about 100 wells.

One of ordinary skill in the art would appreciate that one or more examples or descriptions of kits described herein may be combined to create a kit that includes one or more elements from one or more of the above examples or descriptions. As a non-limiting example, a kit may comprise one or more different types of chambers with one or more different types of moveable covers, such as a kit may include a chamber comprising a multi-well reservoir (e.g. a 96-well plate or 384-well plate as non-limiting examples) that may be sealed by an adhesive sheet (such as a plastic or foil sheet, as non-limiting examples) as well as one or more chambers that may comprise individual tubes or vials with a screw cap as non-limiting examples. A non-limiting example of a kit comprising one or more component (such as one or more RNA stabilizing substances, cellular uptake agents, additive substances, inorganic cations (or salt thereof), activator substance, dissolution substance, buffering agents, water, solvents, or one or more other substances described herein, as non-limiting examples) stored in one type of chamber and one or more other component stored in different type of chamber is shown in FIG. 75C. FIG. 75C shows kit 2000, wherein kit 2000 may comprise package 2001 (such as a walled container, as a non-limiting example). As previously described, package 2001 may comprise moveable cover 2002, hinge 2008, or optional flap or tab 2003 that fits into optional slot 2007 to seal package 2001. Package 2001 may comprise supporting material 2004 to help support or hold chambers 2050 or 2005, wherein supporting material 2004 may be made of cardboard, foam, plastic, paper, or other suitable material to at least partially hold (e.g. support or restrict movement or reduce force or reduce magnitude of vibrations as non-limiting examples) one or more components of kit 2000 during shipping or storage. Kit 2000 may comprise one or more RNA stabilizing substance or combinations of two or more RNA stabilizing substances in chamber 2050 or chamber 2005 as described previously. In some embodiments one or more chamber (e.g. chamber 2050 or chamber 2005) may contain one or more RNA stabilizing substance or combinations of two or more RNA stabilizing substances or may contain one or more additional substances (such as, one or more RNA substance, cellular uptake agents, additive substances, inorganic cations (or salts thereof), activator substance, dissolution substance, buffering agents, water, solvents, or one or more other substances described herein, as non-limiting examples). Chamber 2050 or chamber 2005 may also comprise moveable a cover that may be sealed or resealed as described previously. Kit 2000 may also comprise insert 2006 with instructions for use, such as describing how to mix, store, or dilute one or more components of kit 2000, as non-limiting examples. Kit 2000 may also comprise label 2009 affixed to package 2001, wherein label 2009 may include information about kit 2000, such as what components are included in kit 2000, expiration date, or other pertinent information as non-limiting examples.

One of ordinary skill in the art would appreciate that one or more examples of kits described herein may be packaged in one or more types of packages (such as one or more walled containers as a non-limiting example). In some embodiments a package may comprise one or more walled containers. In some embodiments a package may comprise a walled container wherein a walled container may be a box, bag, envelope, jar, bottle, can, canister, or pouch, as non-limiting examples. As a non-limiting example, a walled container may be made of paper, cardboard, plastic, metal, glass, wood, polymeric material, or other suitable material, as non-limiting examples. In some embodiments a package may comprise a walled container that may be at least partially sealed or covered (such as to prevent at least some of the contents of the walled container from spilling or falling out of the walled container, as a non-limiting example), such as by mechanical interference, sliding seal, zipper, zip seal, zip-top (e.g. Ziploc), press seal, adhesive seal (including tape or adhesive), latch, tab, hook and loop (e.g. Velcro), or moveable cover, or combinations thereof, as non-limiting examples. In some embodiments a moveable cover may be at least partially sealed such as by mechanical interference, sliding seal, zipper, zip seal, zip-top, press seal, adhesive seal, latch, tab, hook and loop, or combinations thereof, as non-limiting examples. As a non-limiting example, a moveable cover may be placed over a walled container (such as by sliding over the walled container, as a non-limiting example) to at least partially cover or seal the contents inside of the package or walled container.

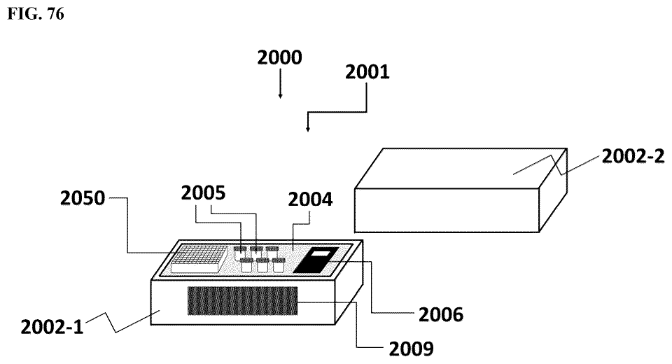

An additional non-limiting example of a kit comprising a package with a walled container and moveable cover is shown in FIG. 76, wherein kit 2000, may comprise package 2001 comprising moveable cover 2002-2 that may at least partially slide over or fit over walled container 2002-1 to at least partially cover or seal the walled container. In some embodiments, moveable cover 2002-2 may comprise a latch, tab, or adhesive seal, as non-limiting examples, (not shown) that helps at least partially fix or adhere moveable cover 2002-2 to walled container 2002-1. As previously described, package 2001 may comprise supporting material 2004 to help at least partially support or hold chambers 2050 or 2005, wherein supporting material 2004 may be made of cardboard, foam, plastic, paper, or other suitable material to at least partially support or hold (e.g. support or restrict movement or reduce force or reduce magnitude of vibrations, as non-limiting examples) one or more components of kit 2000 during shipping or storage. Kit 2000 may comprise one or more RNA stabilizing substance or combinations or mixtures of two or more RNA stabilizing substances in chamber 2050 or chamber 2005 as described previously. In some embodiments one or more chamber (e.g. chamber 2050 or chamber 2005) may contain one or more RNA stabilizing substance or combinations or mixtures of two or more RNA stabilizing substances or may include one or more additional substances such as, one or more RNA substance, cellular uptake agents, additive substances, inorganic cations (or salts thereof), activator substance, dissolution substance, buffering agents, water, solvents, or one or more other substances described herein, as non-limiting examples. Chamber 2050 or chamber 2005 may also comprise a moveable cover that may be sealed or resealed as described previously. Kit 2000 may also comprise insert 2006 with instructions for use, such as describing how to mix, store, or dilute one or more components of kit 2000, as non-limiting examples. Kit 2000 may also comprise label 2009 affixed to package 2001, wherein label 2009 may include information about kit 2000, such as what components are included in kit 2000, expiration date, or other pertinent information as non-limiting examples. As a non-limiting example, package 2001 containing one or more kit components may be at least partially sealed (such as by an adhesive seal (including tape or adhesive), mechanical interference, latch, or tab, or other suitable seal, as non-limiting examples), such as to at least partially prevent one or more kit components from spilling or falling out of the package during transportation or storage, as a non-limiting example.

### Producing a Kit

As non-limiting examples, at least one RNA stabilizing substance of the present disclosure may be produced for later use. A method for producing a kit comprising at least one RNA stabilizing substance may comprise at least one of the steps of preparing or packaging at least one RNA stabilizing substance.

Non-limiting example methods for producing a kit may comprise selecting at least one RNA stabilizing substance or selecting multiple RNA stabilizing substances that may be at least one or more kit components, wherein the RNA stabilizing substances may be from the same or different categories of RNA stabilizing substances described herein. Non-limiting example methods for producing a kit may comprise preparing at least one RNA stabilizing substance by mixing an RNA stabilizing substance with a liquid or diluent to produce a liquid or solution comprising at least one RNA stabilizing substance. A method for producing a kit may further comprise adding or mixing one or more additional substances described herein, such as one or more additional RNA stabilizing substance or auxiliary substance, to produce a kit component comprising at least one RNA stabilizing substance and at least one additional substance. As a non-limiting example, a kit component comprising at least one or more RNA stabilizing substance may be prepared as at least one of an individual RNA stabilizing substance or combinations of two or more RNA stabilizing substances as described herein. As a non-limiting example, a kit component comprising at least one RNA stabilizing substance may be provided as a solid, liquid (e.g. a solution), or gel. Additionally, a method for producing a kit may comprise preparing at least one of an activator substance with specified concentration of inorganic cations or specified pH value as described herein; or preparing a dissolution substance with specified pH value or diluent comprising water with low inorganic cation concentration, as described herein.

A method for producing a kit may comprise adjusting the pH of one or more component to a desired pH or may comprise filtering or sterilizing one or more component prior to placing the components into a chamber or prior to packaging. Additionally, a method for producing a kit may comprise testing one or more components for endotoxin, such as using an LAL assay or other method known in the art. As a non-limiting example, one more kit components may be prepared with an endotoxin level below a specified value as described herein, such as less than 100 endotoxin units/mL as a non-limiting example.

A method for producing a kit may also comprise placing at least one RNA stabilizing substance or a component or mixture comprising at least one RNA stabilizing substance into a chamber. As a non-limiting example, a method may comprise combining one or more components either prior to or after being placed into a chamber. As a non-limiting example, one or more components may be mixed and then placed into a chamber or alternatively, one or more components may be combined inside of a chamber, using one or more steps described.

One or more kit component comprising at least one RNA stabilizing substance or at least one auxiliary substance may be contained in one or more chamber described herein, non-limiting examples include sealable or resealable tubes, vials, bottles, or multi-well reservoirs (e.g. 96-well plates, or 384-well plates) as non-limiting examples. A chamber may also include one or more label as described herein, where a label may be affixed to or printed onto the chamber as non-limiting examples. A label on a chamber may comprise information about the contents of the chamber or other suitable information as described herein. Once an RNA stabilizing substance or kit component is contained in a chamber, a method may comprise sealing said chamber such as with a resealable cover or hermetically sealing said chamber. As non-limiting examples, a method may comprise hermetically sealing a chamber containing an RNA stabilizing substance or kit component and then at least one of packaging or storing said chamber.

A method for producing a kit comprising at least one RNA stabilizing substance may comprise packaging a chamber containing one or more kit components, such as a component comprising at least one RNA stabilizing substance or auxiliary substance, into a walled container as described herein. As a non-limiting example, a walled container may be a box, bag, or other suitable container described herein. A method for producing a kit that comprises packaging one or more kit component comprising at least one RNA stabilizing substance, may also comprise placing a label on the package with information about the contents of the package or placing instructions for use for one more kit component inside of or on the package. As a non-limiting example, instructions for use may be provided as hard copy (such as printed on a piece of paper or plastic) or electronically (such as pdf, an app on a phone or computer, or email, or on the internet, such as via a QR code or URL, as non-limiting examples) or other suitable method described herein. As a non-limiting example, instructions for use may comprise written descriptions of or pictures illustrating one or more steps or process for using one or more kit components as described herein. A method for producing a kit may also comprise sealing a package containing one or more kit components as described herein, such as with an adhesive seal or mechanical interference, such as a latch or tab, or other suitable method described herein. Following packaging of one or more kit components, a method may comprise transporting, shipping, or storing a package containing one or more kit components comprising at least one RNA stabilizing substance, wherein a package may be shipped to a desired location, such as a location of use, or stored at a location of use. As a non-limiting example, an intended use of a kit comprising at least one or more RNA stabilizing substance may be to combine one or more RNA stabilizing substance provided in the kit with one or more RNA substance to produce an RNA stabilizing composition as a non-limiting example.

### Providing a Kit

As non-limiting examples, at least one RNA stabilizing substance of the present disclosure may be provided for later use. A method for providing a kit comprising at least one RNA stabilizing substance may comprise at least one of the steps of selecting, shipping, or transporting at least one RNA stabilizing substance.

Non-limiting example methods for providing a kit may comprise selecting at least one RNA stabilizing substance or selecting multiple RNA stabilizing substances that may be provided as at least one or more kit components, wherein the RNA stabilizing substances may be from the same or different categories of RNA stabilizing substances described herein. As a non-limiting example, the RNA stabilizing substances may be provided as at least one of individual RNA stabilizing substances or combinations of two or more RNA stabilizing substances as described herein. As a non-limiting example, a kit component comprising at least one RNA stabilizing substance may be provided as a solid, liquid (e.g. a solution), or gel.

Additionally, a method for providing a kit comprising at least one RNA stabilizing substance or at least one auxiliary substance may comprise providing a component with an endotoxin level below a specified value. As a non-limiting example, one more kit components may be provided with an endotoxin level below a specified value as described herein, such as less than 100 endotoxin units/mL as a non-limiting example.

A method for providing a kit comprising at least one or more RNA stabilizing substance may comprise providing at least one RNA stabilizing substance in a chamber. As a non-limiting example, one or more kit components comprising at least one RNA stabilizing substance or at least one auxiliary substance may be provided in a chamber such as sealable or resealable tubes, vials, bottles, or multi-well reservoirs (e.g. 96-well plates, or 384-well plates) as non-limiting examples. A chamber comprising at least one RNA stabilizing substance may be sealed such as with a resealable cover or hermetically sealed, wherein a method may comprise providing one more kit component in a hermetically sealed chamber or with a resealable cover. A chamber may also include one or more label as described herein, where a label may be affixed to or printed onto the chamber as non-limiting examples. A label on a chamber may comprise information about the contents of the chamber or other suitable information as described herein.

A method for providing a kit comprising at least one RNA stabilizing substance may comprise providing a chamber containing one or more kit components, such as a component comprising at least one RNA stabilizing substance or auxiliary substance, in a package, such as a walled container as described herein. As a non-limiting example, a walled container may be a box, bag, or other suitable container described herein. A method may also comprise placing a label on the package with information about the contents of the package. Additionally, a method for providing a kit comprising at least one RNA stabilizing substance, may also comprise providing instructions for use as described herein. As a non-limiting example, instructions for use may be packaged and provided with one or more kit components in a package, such as hard copy instructions, or instructions may be provided electronically (such as on the internet or pdf as non-limiting examples) or provided by one or more other suitable method as described herein. As a non-limiting example, instructions for use may comprise instructions for mixing or storing one or more kit components with an RNA substance, such as mixing at least one RNA substance with a kit component comprising at least one RNA stabilizing substance.

As a non-limiting example, instructions for use may also comprise instructions for adding an activator substance to an RNA stabilizing composition to increase viscosity prior to storage or stability testing. As a non-limiting example, instructions for use may also comprise instructions for adding a dissolution substance to a composition following storage or prior to analysis as described herein.

A method for providing a kit comprising at least one RNA stabilizing substance may comprise at least one of transporting, shipping, or storing a package containing one or more kit components comprising at least one RNA stabilizing substance, wherein a package may be transported or shipped to a desired location, such as a location of use, or stored at a location of use. As a non-limiting example, an intended use of a kit comprising at least one or more RNA stabilizing substance may be to combine one or more RNA stabilizing substance provided in the kit with one or more RNA substance to produce an RNA stabilizing composition as a non-limiting example.

In some embodiments one or more method may comprise providing a kit comprising at least one or more RNA stabilizing substance to produce a composition comprising a combination of at least one or more RNA substance and at least one or more RNA stabilizing substance.

In some embodiments one or more method may comprise providing a kit comprising one or more RNA stabilizing substance, wherein an RNA stabilizing substance provided in a kit may be provided in a chamber (e.g. a vial, tube, 96-well plate, or 384-plate as non-limiting examples) to produce a composition comprising one or more RNA stabilizing substance and one or more RNA substance. In some embodiments one or more method may comprise providing a kit comprising one or more RNA stabilizing substance, wherein one or more RNA stabilizing substance provided in a kit may be provided in a chamber as an individual RNA stabilizing substance or as a combination of two or more RNA stabilizing substances (such as a premixed composition comprising a combination of two or more RNA stabilizing substances as a non-limiting example) to produce a composition comprising one or more RNA stabilizing substance and one or more RNA substance. As non-limiting examples, one or more RNA stabilizing substance may be provided in individual chambers or provided in separate wells of a multi-well reservoir, or combinations thereof.

In some embodiments one or more method may comprise providing a kit comprising one or more RNA stabilizing substance, wherein a kit may comprise instructions for use (such as instructions for mixing or otherwise combining one or more RNA stabilizing substance with one or more RNA substance to produce a composition comprising one or more RNA stabilizing substance and one or more RNA substance as a non-limiting example). As non-limiting examples, instructions for use may comprise one or more instructions described herein, including, but not limited to, instructions for one or more of: storing a composition comprising at least one or more RNA substance and one or more RNA stabilizing substance at one or more specified temperature, or performing accelerated stability testing or screening, or mixing (or otherwise combining) or storing a composition comprising one or more RNA substance and one or more RNA stabilizing substance or one or more additional kit components (non-limiting examples, including one or more cellular uptake agent or one or more auxiliary substance), or mixing (or otherwise combining) an activator substance with one or more RNA stabilizing composition, or mixing (or otherwise combining) a dissolution substance with one or more RNA stabilizing composition, or measuring or analyzing RNA stability following storage of one or more RNA stabilizing composition, or performing reverse transcription using a reverse transcriptase enzyme following storage of one or more RNA stabilizing composition, or analyzing or comparing RNA stability of one or more control RNA (such as an RNA encoding a fluorescent or bioluminescent protein, as non-limiting examples), or combinations thereof.

In some embodiments, one or more methods may also comprise providing a kit comprising one or more RNA stabilizing substance or one or more additional substance (such as one or more auxiliary substance as a non-limiting example), wherein one or more RNA stabilizing substance or one or more additional substance provided in a kit may be provided in a chamber, where one or more RNA stabilizing substance or one or more additional substance may be provided in a chamber as individual substances or as a combination of one or more RNA stabilizing substance and one or more additional substance (such as, non-limiting examples, a premixed composition comprising a combination of one or more RNA stabilizing substance and one or more auxiliary substance (such as a buffering agent or cellular uptake agent, as non-limiting examples)) to produce a composition comprising one or more RNA substance and one or more RNA stabilizing substance.

In some embodiments, one or more methods may also comprise providing a kit comprising one or more RNA stabilizing substance or two or more RNA stabilizing substances, wherein one or more RNA stabilizing substance or two or more RNA stabilizing substances provided in a kit may be provided in a chamber. As a non-limiting example, one or more RNA stabilizing substance may be provided as a combination or mixture with one or more additional substance described herein (such as one or more additional RNA stabilizing substance, buffering agent, or auxiliary substance, as non-limiting examples) and provided in a chamber to produce a composition comprising one or more RNA substance and one or more RNA stabilizing substance.

In some embodiments one or more method may comprise providing a kit comprising one or more RNA stabilizing substance, wherein one or more RNA stabilizing substance provided in a kit may be provided in a chamber where a chamber may have at least one label. As non-limiting examples, a label on a chamber may include information about the substance or composition within the chamber as described herein (such as the composition, individual components or substances, or concentration of substances, as non-limiting examples) or a label may be coded, such as by an alphanumeric code (e.g. A-Z, or 1-1000, or combinations thereof such as A1, B102, or C₃₀₁ as non-limiting examples), date or time codes, or lot or batch numbers, as non-limiting examples, or combinations thereof. As a non-limiting example, one or more additional substance (such as an auxiliary substance as a non-limiting example) may also be provided in a kit, wherein one or more additional substance may be provided in chamber where a chamber may have at least one label.

In some embodiments one or more method may comprise providing a kit comprising one or more RNA stabilizing substance, wherein a kit may also comprise one or more auxiliary components described herein. As non-limiting examples an auxiliary component may comprise one or more auxiliary substances described herein (e.g. a buffer, cellular uptake agent, or inorganic cation, as non-limiting examples), one or more activator substances or dissolution substances described herein, or one or more empty chambers that may be used for mixing or storage, or prefilled or empty syringes, or spin columns (including desalting, buffer exchange, or nucleic acid purification columns, as non-limiting examples), or filters (e.g. a filter between about 0.1-1 μm, such as an about 0.22 μm filter or about 0.45 μm filter, as non-limiting examples), one or more additional enzymes or reagents (such as a reverse-transcriptase enzyme or associated buffers or nucleotides, or an RNA loading dye or other materials to prepare compositions for analysis by gel electrophoresis, as non-limiting examples), or one or more control RNA as described herein (e.g. GFP or firefly luciferase as non-limiting examples), or combinations thereof.

In some embodiments one or more method may comprise filtering one or more kit components prior to packaging, wherein one or more RNA stabilizing substance in a kit may be filtered (e.g. filtered using a filter between about 0.1-1 μm, such as an about 0.22 μm filter or an about 0.45 μm filter, as non-limiting examples) or sterilized prior to packaging (such as by autoclave, filter sterilization, steam, heat, or other methods known in the art as non-limiting examples) or sterilized after packaging as described herein, or combinations thereof.

In some embodiments, one or more methods may also comprise assembling or packaging a kit comprising one or more RNA stabilizing substance, wherein a kit comprising one or more RNA stabilizing substance may be provided to produce a composition comprising a combination of at least one or more RNA substance and at least one or more RNA stabilizing substance. As a non-limiting example, assembling or packaging a kit may comprise placing one or more kit components (such as one or more RNA stabilizing substance or auxiliary substance) in a chamber and providing instructions for use, wherein one or more kit components (as non-limiting examples, such as one or more chamber containing one or more RNA stabilizing substance or auxiliary substance, or instructions for use (e.g. an insert, sheet of paper, or pamphlet, as non-limiting examples)) may be placed in a package wherein a package may comprise a walled container. As a non-limiting example, a package containing one or more kit components may be at least partially covered or sealed (such as with an adhesive seal, tape, mechanical interference, latch, or tab, or other suitable seal, as non-limiting examples), such as to at least partially prevent one or more kit components from spilling or falling out of the package during transportation or storage as a non-limiting example.

As a non-limiting example, one or more kit components, (such as one or more RNA stabilizing substance or auxiliary substance as non-limiting examples) or a composition comprising one or more substances described herein (such as a composition comprising one or more RNA stabilizing substance or auxiliary substance as non-limiting examples) may be provided comprising a liquid (such as a solution or suspension, as a non-limiting examples), or as a solid (such as a powder, tablet, or crystals as non-limiting examples), or as a gel (such as a hydrogel as a non-limiting example), or combinations thereof.

As a non-limiting example, a kit or package may comprise a walled container, wherein a walled container may comprise one or more label. As non-limiting examples a label may comprise information about the contents of a kit or one or more kit components, or instructions for use for one or more kit components, or one or more potential use of the kit (such as RNA stability testing, accelerated RNA stability screening, or RNA stability and lipid compatibility screening, as non-limiting examples), or relevant manufacturing or storage information such as lot numbers, trace codes or numbers, expiration dates, storage conditions (e.g. storage temperature), manufacturing date, manufacturer or manufacturing location, or company designs or logos; or combinations thereof, that may provide relevant information about the kit source, storage conditions, or implementation of the kit to the user.

In some embodiments one or more method may comprise providing a kit comprising one or more RNA stabilizing substance, wherein a kit may be transported, shipped, or stored. As non-limiting examples, a kit may be transported or shipped by airplane, automobile (such as truck, van, or car as non-limiting examples) or boat, or combinations thereof. In some embodiments a kit may be provided wherein a kit may be transported, shipped, or stored at about 20° C. or greater. In some embodiments a kit may be provided wherein a kit may be transported, shipped, or stored at about −80° C. or greater, or at about −60° C. or greater, at about −40° C. or greater, or at about −20° C. or greater, or about −10° C. or greater, or about 0° C. or greater, or about 4° C. or greater, or about 10° C. or greater, or about 20° C. or greater. As a non-limiting example, transporting or shipping a kit may comprise transporting a kit to one or more desired destination (such as by car, truck, airplane, boat, or other suitable transportation method, as non-limiting examples). As a non-limiting example, storing a kit may comprise keeping an assembled or packaged kit in a warehouse or desired location (such as a location of use as a non-limiting example) for a period of time (e.g. at least 1 hr, or at least 1 day, or at least 1 week, or at least 1 month, as non-limiting examples), wherein a kit may be stored at the location of use or transported to the desired destination as non-limiting examples.

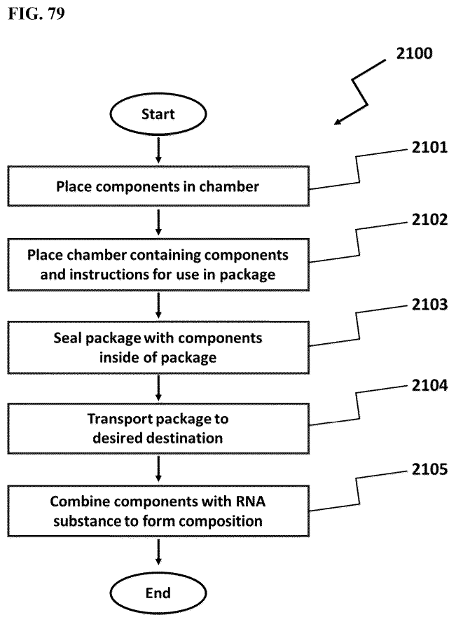

FIG. 79 is a flowchart that summarizes a non-limiting example method 2100 for producing and using a kit comprising at least one or more RNA stabilizing substance in accordance with the present disclosure. The method 2100 may be initiated by placing one or more kit components (such as an RNA stabilizing substance or an auxiliary substance, as non-limiting examples) in a chamber (2101). The chamber may be any suitable chamber as described herein and may be, as non-limiting examples, a single use or multiuse vial, sealable or resealable tube with screw-cap or friction-fit cap, multi-well reservoir with sealable or resealable moveable cover, 96-well plate, 384-well plate, bottles, containers, vials, tubes, syringes (including prefilled or single use syringes), blisters, capsules, or cartridges. The components may be placed in a chamber by any method known in the art. As non-limiting examples, one or more components may be placed in a chamber by pipetting, pouring, using a syringe, injection, or the like, or by adding lyophilized or solid pellets, by measuring powders, or by any other suitable method. One or more chamber containing the desired kit components may then be placed inside of a package (2102). The package may be any suitable package for shipping or storage, such as, a non-limiting example, a walled container wherein a walled container may be a box, bag, envelope, jar, bottle, can, canister, or pouch and a walled container may be made of paper, cardboard, plastic, metal, wood, glass, polymeric material, or other suitable material as non-limiting examples. Instructions for use may also be provided or placed inside of the package (2102), and may comprise an insert, sheet of paper, or pamphlet describing how to mix, store, or dilute one or more components of the kit, as non-limiting examples. The package containing one or more kit components may then be sealed (2103) by any suitable method, as non-limiting examples, such as by using adhesive, tape, string, rope, strap, or mechanical interference (e.g. a latch or tab as non-limiting examples). The package may then be stored at a location of use or transported to a desired destination or location of use (2104). Depending on the specific implementation, the package or one or more components may be refrigerated, frozen, or otherwise maintained in a temperature-controlled environment during transportation or storage. One or more kit components comprising at least one or more RNA stabilizing substance or one or more auxiliary substance may then be combined with one or more RNA substance to produce a composition comprising one or more RNA substance and at least one or more RNA stabilizing substance (2105). In some embodiments an RNA substance may be provided at the location of use, wherein a kit comprising one or more RNA stabilizing substance or one or more auxiliary substance may be provided to produce a composition comprising a combination of one or more RNA substance and one or more RNA stabilizing substance as described herein.

As a non-limiting example, a composition produced with one or more kit components comprising one or more RNA substance and one or more RNA stabilizing substance may be stored at or above freezing temperatures (e.g. at or above about −80° C., or −20° C., or 0° C., or 4° C., or 10° C., or 20° C. as non-limiting examples) or used for one or more desired applications, such as improving or screening RNA stability of one or more RNA substances in or more RNA stabilizing composition, or improving or screening RNA stability or compatibility of one or more RNA substance and one or more cellular uptake agent in one or more RNA stabilizing composition, as non-limiting examples.

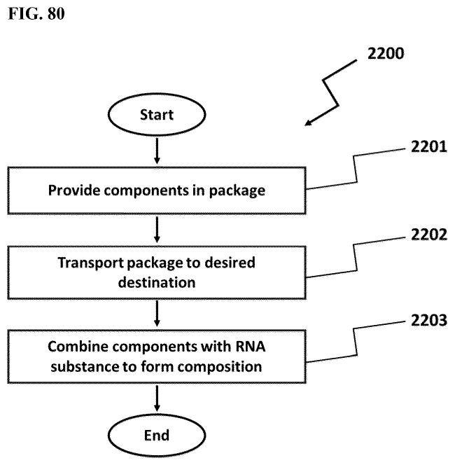

FIG. 80 is a flowchart that summarizes a non-limiting example method 2200 for providing and using a kit comprising at least one or more RNA stabilizing substance in accordance with the present disclosure. The method 2200 may be initiated by providing (2201) one or more kit components (such as an RNA stabilizing substance, an auxiliary substance, or instructions for use) to produce a composition comprising one or more RNA substance and one or more RNA stabilizing substance. One or more kit components may be provided in a package such as a walled container or other suitable package as described herein. One or more kit components (such as an RNA stabilizing substance or an auxiliary substance, as non-limiting examples) may be provided in a chamber such as sealable or resealable tubes, vials, bottles, or multi-well reservoirs (e.g. 96-well plates, or 384-well plates) as non-limiting examples. The components may then be stored at the location of use or transported (2202) to the desired destination or location of use. Depending on the specific implementation, the package or one or more components may be refrigerated, frozen, or otherwise maintained in a temperature-controlled environment during transportation or storage. One or more kit components comprising at least one or more RNA stabilizing substance or one or more auxiliary substance may then be combined with one or more RNA substance to produce a composition comprising one or more RNA substance and at least one or more RNA stabilizing substance (2203). In some embodiments an RNA substance may be provided at the location of use, wherein a kit comprising one or more RNA stabilizing substance or one or more auxiliary substance may be provided to produce a composition comprising a combination of one or more RNA substance and one or more RNA stabilizing substance as described herein.

As a non-limiting example, a composition produced with one or more kit components comprising one or more RNA substance and one or more RNA stabilizing substance may be stored at or above freezing temperatures (e.g. at or above about −80° C., or −20° C., or 0° C., or 4° C., or 10° C., or 20° C. as non-limiting examples) or used for one or more desired applications, such as improving or screening RNA stability of one or more RNA substances in or more RNA stabilizing composition, or improving or screening RNA stability or compatibility of one or more RNA substance and one or more cellular uptake agent in one or more RNA stabilizing composition, as non-limiting examples.

A non-limiting method of use may comprise at least the step of mixing at least one RNA stabilizing substance from a kit with at least one RNA substance. A non-limiting method of use may comprise storing a composition and evaluating the stability of at least one RNA substance in a composition comprising at least one RNA stabilizing substance and at least one RNA substance. A non-limiting method of use for evaluating RNA stability may comprise the step of mixing at least one RNA stabilizing substance from a kit with at least a first RNA substance and may further comprise the step of exposing the mixture to a physical condition (a non-limiting example is exposing the mixture to a temperature condition for a selected duration) and may comprise the step of measuring at least one property that indicates the stability of the RNA in the composition.

As a non-limiting example, in some embodiments a kit may be provided comprising one or more RNA stabilizing substance, where an RNA substance may be provided at a location of use to produce a composition comprising one or more RNA substance and one or more RNA stabilizing substance with one or more of the provided kit components as described herein.

One of ordinary skill in the art would appreciate that one or more examples, methods, or descriptions of kits, or compositions (e.g. compositions comprising one or more RNA substance and one or more RNA stabilizing substance as non-limiting examples) described herein may be combined to create a kit, method, process, or composition that may include one or more elements from one or more of the above examples or descriptions.

As a non-limiting example, a kit may comprise an RNA stabilizing substance and one or more cellular uptake agent (such as one or more lipid or ionizable lipid as a non-limiting example), and instructions for use describing how to mix one or more RNA substance with one or more RNA stabilizing substance and one or more cellular uptake agent to produce an RNA stabilizing composition; this same kit may also comprise one or more activator or dissolution substance and instructions for use describing how to mix an activator or dissolution substance with one or more RNA stabilizing composition comprising a cellular uptake agent along with instructions for performing accelerated stability testing or screening as well as instructions for measuring or analyzing RNA stability following storage of one or more RNA stabilizing composition comprising one or more kit components, as non-limiting examples.

As another non-limiting example, a method may comprise providing a kit comprising one or more RNA stabilizing substance, wherein an RNA stabilizing substance provided in a kit may be provided in a chamber along with instructions for use for mixing one or more RNA stabilizing substance with one or more RNA substance to produce an RNA stabilizing composition; this same method may also comprise assembling and packaging one or more kit components, wherein a kit component may also include one or more empty chambers (e.g. empty tubes or vials, as non-limiting examples) and instructions for performing accelerated stability testing or screening as well as instructions for measuring or analyzing RNA stability following storage of one or more RNA stabilizing composition comprising one or more kit components, as non-limiting examples.

### Kit Components and Definitions

As used herein a walled container is a container with at least two walls or two sides that create an at least partly-enclosed cavity wherein the contents inside of the walled container may be at least partially sealed or the contents at least partially prevented from falling or spilling out of either the walled container alone or when a cover (e.g. a moveable cover as a non-limiting example) is placed over the walled container. As a non-limiting example, a walled container may include one or more sides that is a triangle, square, rectangle, parallelogram, circle, ellipse, pentagon, hexagon, octagon, or other suitable shape to create an at least partly-enclosed cavity. As a non-limiting example, a walled container may be a pyramid, cube, cuboid, cylinder, sphere, parallelepiped, prism, or cone, or other suitable shape to create an at least partly-enclosed cavity. As non-limiting example a walled container may be a box, bag, envelope, jar, bottle, can, canister, or pouch. As non-limiting examples a walled container may be made of paper, cardboard, plastic, metal, glass, wood, polymeric material, or other suitable material as non-limiting examples. As a non-limiting example, a walled container may be at least partially sealed or covered (such as to prevent at least some of the contents of the walled container from spilling or falling out of the walled container as a non-limiting example), such as by a moveable cover, mechanical interference, sliding seal, zipper, zip seal, zip-top (e.g. Ziploc), press seal, adhesive seal, latch, tab, hook and loop (e.g. Velcro), or combinations thereof as non-limiting examples.

As used herein, an adhesive seal may include adhesive, or material comprising adhesive, such as tape or an adhesive label, or plastic, paper, cardboard, metal, glass, wood, polymeric material, or other material that may comprise an adhesive, as non-limiting examples

As used herein a moveable cover is a cover (e.g. a cap, plug, top, seal, or lid, as non-limiting examples) that can be placed over an opening to at least partially seal or at least partially prevent the contents inside of the opening from spilling, falling out or leaking, wherein the cover may be at least partially opened or removed to at least partially access some of the contents inside of the opening. As a non-limiting example, a moveable cover may also be put back into place after at least partially removing the cover to at least partially seal or reseal the contents inside of the opening. As a non-limiting example, a moveable cover may comprise a screw-top, screw-cap, friction-fit cap or plug, a top to a box, lid, foldable sheet, zipper, zip-top (e.g. Ziploc seal), hook and loop (e.g. Velcro), adhesive sheet (e.g. tape, plastic, or foil sheet) or other suitable cover that may be either permanently removed, at least partially removed, or temporarily removed and at least partially put back into place over an opening.

As used herein an auxiliary component is a component or substance that may be included as one or more parts of a kit. As non-limiting examples an auxiliary component in a kit may comprise one or more auxiliary substance, wherein an auxiliary substance may be an individual auxiliary substance or a combination of two or more auxiliary substances (e.g. a cellular uptake agent and a buffering agent, or an activator substance and an inorganic cation (or salt thereof), or a buffering agent and an inorganic cation (or salt thereof), as non-limiting examples). As non-limiting examples an auxiliary component may also include one or more empty chambers (such as one or more empty tube, 96-well plate, or vial as non-limiting examples) that may be used for mixing or storage, or prefilled or empty syringes, or spin columns (as non-limiting examples desalting, buffer exchange, or size exclusion columns, or nucleic acid purification columns such as silica or silica gel columns, as non-limiting examples), or filters (a non-limiting example of a filter may be a sterile or non-sterile filter, such as an about 0.22 μm filter, or about 0.45 μm filter, or filter between about 0.1-1 μm as non-limiting examples), or one or more additional enzymes or reagents (such as a reverse-transcriptase enzyme and associated buffers and nucleotides, or an RNA loading dye or other materials to prepare compositions for analysis by gel electrophoresis, as non-limiting examples), or combinations thereof. As another non-limiting example, an auxiliary component may include a control RNA such as an RNA encoding a fluorescent or bioluminescent protein (such as green fluorescent protein (GFP), enhanced GFP (eGFP), red fluorescent protein (RFP), mCherry, or firefly luciferase as non-limiting examples).

As used herein an auxiliary substance in a kit, is a substance that may be included as one or more parts or components of a kit in addition to one or more RNA stabilizing substance. As non-limiting examples, an auxiliary substance in a kit, may comprise one or more cellular uptake agents, additive substances, inorganic cations (or salts thereof), RNA substances (e.g. a control RNA as a non-limiting example), activator substances, dissolution substances, buffering agents, water, solvents, diluents, or combinations thereof, as non-limiting examples. As a non-limiting example an auxiliary substance may be provided as an individual auxiliary substance or a combination of two or more auxiliary substances (e.g. a cellular uptake agent and a buffering agent, or an activator substance and an inorganic cation (or salt thereof), or a buffering agent and an inorganic cation (or salt thereof), as non-limiting examples).

All cited sources, for example, references, publications, and art cited herein, are incorporated into this application by reference, even if not expressly stated in the citation. In the case of a conflicting statement of a cited source and the instant application, the statement in the instant application shall control.

### EXAMPLES

The following non-limiting examples describe examples of the invention in more detail and in no way are to be construed as limiting the scope thereof. Additionally, the following non-limiting examples are used to show candidate uses of materials and implementation of the invention and is in no way meant to limit the scope of use of the invention.

The following examples of modified carbohydrates may include one or more type of carbohydrate. The following examples also describe using one or more types of carbohydrates to create a modified carbohydrate. One of the modified carbohydrates discovered to stabilize RNA is modified starch. As known to those skilled in the art, starch is a polysaccharide made of amylose (a linear polymer of glucose), amylopectin (a branched polymer), or a combination of both amylose and amylopectin. Those skilled in the art will recognize that, as a non-limiting example, potato starch is a commonly used example starch. As non-limiting examples of modified carbohydrate starches, potato starch from Sigma-Aldrich was used as described herein to synthesize modified starches used in compositions with RNA to produce RNA stabilizing compositions. Another non-limiting example starch known to those skilled in the art is potato starch treated according to Zulkowsky (treated with glycerol at 190° C.) (herein referred to as Zulkowsky starch), obtained from Sigma-Aldrich for modification to synthesize non-limiting examples of modified starches used in compositions with RNA to produce RNA stabilizing compositions.

Among the materials used in the following examples include the following materials: Potato starch (Sigma-Aldrich Product #S9765); Zulkowsky starch, from potato (Sigma-Aldrich Product #85642); Inositol (myo-inositol) (Sigma-Aldrich Product #57570); Lactic Acid (lactic acid solution, ≥85%) (Sigma-Aldrich Product #252476); Malic acid (Sigma-Aldrich Product #02288); Sucrose (Sigma-Aldrich Product #84097); D-Glucose (Sigma-Aldrich Product #G5767); D-Sorbitol (Sigma-Aldrich Product #S1876); Glycerol (Sigma-Aldrich Product #G5516); Citric acid (Sigma-Aldrich Product #251275); Trimethylglycine hydrochloride (TMG-HCl) (also known as betaine-HCl) (Sigma-Aldrich Product #B3501); Trehalose (Cayman Chemical, Product #20517); Na-Carboxymethylcellulose (CMC) ˜90 kDa (Sigma-Aldrich, Product #419273); Polyquaternium-10 (Divinity Cosmetic Supply, Port St Lucie, FL); Kappa Carrageenan (Modernist Pantry, Product #1011-50); Lambda Carrageenan (Modernist Pantry, Product #1060-50); Fucoidan (Cayman Chemical, Product #20357); Hyaluronic Acid (Pure Health Botanicals, LLC); Gellan Gum F-Low Acyl(Modernist Pantry, Product #1028); Xanthan Gum (Modernist Pantry, Product #1019-50); Na-Alginate Pantry, (Modernist Product #1007-50); Diethylaminoethyl-Dextran ˜10 kDa (DEAE-Dextran) (Sigma-Aldrich Product #00898); Beta-Cyclodextrin (L′Eternal World, LLC, Cleveland, OH, dba as ebay leternelworld98); Gamma-Cyclodextrin (L′Eternal World, LLC, Cleveland, OH, dba as ebay leternelworld98); Guar Hydroxypropyltrimonium Chloride (Cationic Guar, GuarCat) (Lotioncrafters, Eastsound, WA).

### Example 1

### Synthesis of Phosphate Modified Carbohydrates-Herein Referred to as Protocol 1

Synthesis of phosphate modified carbohydrates was performed according to the following reference: L. Passauer, F. Liebner, K. Fischer, Synthesis and Properties of Novel Hydrogels from Cross-linked Starch Phosphates, Macromolecular Symposia. 244 (2006) 180-193.

### Briefly:

Phosphate modified starch was synthesized by mixing 0.5 g of potato starch (Sigma-Aldrich, St. Louis, MO; Product #S9765) with 3 mL of a sodium phosphate solution (pH 5-6) containing about 1 g of sodium phosphate (sodium phosphate monobasic (Sigma-Aldrich, Product #74092) and sodium phosphate dibasic solution (Sigma-Aldrich, Product #94046)). The starch and phosphate mixture was then gently mixed for 30 min at room temperature to create a uniform slurry. The slurry was then dried at 120° F.-140° F. Following drying, the mixture was heated to about 300° F. for 3 hrs. Following heating, the dried cake was washed 3 times with 2 mL of ethanol and allowed to dry overnight. The following day, the cake was resuspended in molecular biology grade water at the desired concentration (typically 500-250 mg/mL) and the pH of the suspension was adjusted to 7 using NaOH. This solution was used in future RNA stability tests and referred to as Na—PO4 Starch 300F.

Protocol 1 was used to produce several variations of phosphate modified carbohydrates using sodium phosphate as listed in Table 1. Depending on the type of carbohydrate used, the time and temperature of heating was selected as shown in Table 1.

TABLE 1
Sodium Phosphate Modified Carbohydrates
Produced Using Protocol 1

| | Heating | Heating | |
|---|---|---|---|
| Carbohydrate | Temperature | Time | Reference Name |
| Inositol | 300° F. | 3 hrs | Na—PO4 Inositol 300 F. |
| Inositol | 280° F. | 2 hrs | Na—PO4 Inositol 280 F. |
| Sorbitol | 280° F. | 2 hrs | Na—PO4 Sorbitol 280 F. |
| Starch | 280° F. | 2 hrs | Na—PO4 Starch 280 F. |
| Starch | 250° F. | 2 hrs | Na—PO4 Starch 250 F. |
| Sucrose | 200° F. | 2 hrs | Na—PO4 Sucrose 200 F. |
| Glucose | 200° F. | 2 hrs | Na—PO4 Glucose 200 F. |
| Zulkowsky | 250° F. | 2 hrs | Na—PO4 Zulkowsky Starch |
| | | | 250 F. |
| Zulkowsky | 220° F. | 2 hrs | Na—PO4 Zulkowsky Starch |
| | | | 220 F. |

Protocol 1 was also used to produce several variations of phosphate modified carbohydrates using potassium phosphate as listed in Table 2. Depending on the type of carbohydrate used, the time and temperature of heating was selected as shown in Table 2.

TABLE 2
Potassium Phosphate Modified Carbohydrates
Produced Using Protocol 1

| | Heating | Heating | |
|---|---|---|---|
| Carbohydrate | Temperature | Time | Reference Name |
| Inositol | 300° F. | 2 hrs | K—PO4 Inositol 300 F. |
| Sorbitol | 300° F. | 2 hrs | K—PO4 Sorbitol 300 F. |
| Starch | 300° F. | 2 hrs | K—PO4 Starch 300 F. |
| Starch | 250° F. | 2 hrs | K—PO4 Starch 250 F. |
| Sucrose | 200° F. | 2 hrs | K—PO4 Sucrose 200 F. |
| Glucose | 200° F. | 2 hrs | K—PO4 Glucose 200 F. |
| Zulkowsky | 200° F. | 2 hrs | K—PO4 Zulkowsky Starch |
| | | | 200 F. |

### Example 2

### Production of Cationic Quaternary Ammonium Modified Carbohydrates-Herein Referred to as Protocol 2

### Synthesis of Cationic Quaternary Ammonium Modified Carbohydrates—Using Trimethylglycine-HCl

Synthesis of cationic quaternary ammonium modified carbohydrates was performed according to the following reference: “N. Karić, M. Vukčević, M. Ristić, A. Perić-Grujić, A. Marinković, K. Trivunac, A green approach to starch modification by solvent-free method with betaine hydrochloride, Int J Biol Macromol. 193 (2021) 1962-1971.”

### Briefly:

Cationic quaternary ammonium modified starch was synthesized by mixing 0.5 g of soluble potato starch with 1 g trimethyl glycine hydrochloride (TMG-HCl) (also known as betaine-HCl) (Sigma-Aldrich, St. Louis, MO; Product #B3501). Then 1-2 mL of water, supplemented with 0.025 mL lactic acid solution (≥85%) (Sigma-Aldrich, St. Louis, MO; Product #252476), was added to the starch TMG-HCl mixture, to produce a semi wet paste. The resulting paste was then heated to 200° F. for 1 hr and the water was allowed to evaporate. Following heating, the dried paste was washed 3 times with 2 mL of ethanol and allowed to dry overnight. The following day, the resulting dried paste was resuspended in molecular biology grade water at the desired concentration (typically 500-250 mg/mL) and the pH of the suspension was adjusted to 7 using NaOH. This solution was used in future RNA stability tests and referred to as Cationic-Starch 200F.

Protocol 2 was used to produce several variations of cationic quaternary ammonium modified carbohydrates using TMG-HCl as listed in Table 3. Depending on the type of carbohydrate used, the time and temperature of heating was selected as shown in Table 3.

TABLE 3
Cationic Quaternary Ammonium Modified
Carbohydrates Produced Using Protocol 2

| | Heating | Heating | |
|---|---|---|---|
| Carbohydrate | Temperature | Time | Reference Name |
| Inositol | 300° F. | 1 hr | Cationic-Inositol 300 F. |
| Inositol | 280° F. | 1 hr | Cationic-Inositol 280 F. |
| Sorbitol | 300° F. | 1 hr | Cationic-Sorbitol 300 F. |
| Sorbitol | 250° F. | 1 hr | Cationic-Sorbitol 250 F. |
| Starch | 220° F. | 1 hr | Cationic-Starch 220 F. |
| Starch | 200° F. | 1 hr | Cationic-Starch 200 F. |
| Sucrose | 200° F. | 1 hr | Cationic-Sucrose 200 F. |
| Glucose | 200° F. | 1 hr | Cationic-Glucose 200 F. |
| Zulkowsky | 200° F. | 1 hr | Cationic-Zulkowsky Starch |
| | | | 200 F. |

### Example 3

### Production of Dual Cationic Quaternary Ammonium and Phosphate Modified Carbohydrates-Herein Referred to as Protocol 3.

Protocol 1 and was used to further modify a cationic quaternary ammonium modified starch with phosphate in addition to the previously added cationic quaternary ammonium modification.

### Briefly:

Dual cationic quaternary ammonium and phosphate modified starch was synthesized by mixing 0.1 g of Cationic-Starch 220F with 0.5 mL of a sodium phosphate solution (pH 5-6) containing about 0.2 g of sodium phosphate. The starch and phosphate mixture was then gently mixed for 30 min at room temperature to create a uniform slurry. The slurry was then dried at 120° F.-140° F. Following drying, the mixture was heated to about 240° F. for 2 hrs. Following heating, the dried cake was washed 3 times with 2 mL of ethanol and allowed to dry overnight. The following day, the cake was resuspended in molecular biology grade water at the desired concentration (typically 500-250 mg/mL) and the pH of the suspension was adjusted to 7 using NaOH. This solution was used in future RNA stability tests and referred to as Dual Cationic-PO4 Starch.

Protocol 3 was also used to modify cationic-Inositol by mixing 0.1 g of Cationic-Inositol 300F with 0.5 mL of a sodium phosphate solution (pH 5-6) containing about 0.2 g of sodium phosphate and heating to 300° F. for 2 hrs. This solution was used in future RNA stability tests and referred to as Dual Cationic-PO4 Inositol.

### Example 4

### Production of Dual Phosphate and Cationic Quaternary Ammonium Modified Carbohydrates—Herein Referred to as Protocol 4.

Protocol 2 was used to further modify a phosphate modified starch with a cationic quaternary ammonium in addition to the previously added phosphate modification.

### Briefly:

Dual phosphate and cationic quaternary ammonium modified starch was synthesized by mixing 0.1 g of Na—PO4 Starch 300F with 0.2 g trimethyl glycine hydrochloride (TMG-HCl) (also known as betaine-HCl). Then 0.2-0.4 mL of water, supplemented with 0.05 mL lactic acid solution, was added to the starch TMG-HCl mixture, to produce a semi wet paste. The resulting paste was then heated to 220° F. for 1 hr and the water was allowed to evaporate. Following heating, the dried paste was washed 3 times with 2 mL of ethanol and allowed to dry overnight. The following day, the resulting dried paste was resuspended in molecular biology grade water at the desired concentration (typically 500-250 mg/mL) and the pH of the suspension was adjusted to 7 using NaOH. This solution was used in future RNA stability tests and referred to as Dual PO4-Cationic Starch.

Protocol 4 was also used to modify phosphate-Inositol by mixing 0.1 g of Na—PO4-Inositol 300F with 0.2 g trimethyl glycine hydrochloride (TMG-HCl) and then adding 0.2-0.4 mL of water, supplemented with 0.05 mL lactic acid solution and heating to 220° F. This solution was used in future RNA stability tests and referred to as Dual PO4-Cationic Inositol.

### Example 5-A

### Production of Additional Modified Polysaccharides

Additional phosphate modified polysaccharides were synthesized using Protocol 1. The type of polysaccharide used and selected time and temperature variations are listed in Table 4-1.

TABLE 4-1
Phosphate Modified Carbohydrates Produced Using Protocol 1

| | Heating | Heating | | |
|---|---|---|---|---|
| Carbohydrate | Temperature | Time | | Reference Name |
| Carboxymethylcellulose | 250° F. | 1 | hr | Na—PO4-CMC 250 F. |
| (CMC) | | | | |
| Fucoidan | 250° F. | 1 | hr | Na—PO4-Fucoidan |
| | | | | 250 F. |
| Polyquaternium-10 | 250° F. | 1 | hr | Na—PO4-Polyquat-10 |
| | | | | 250 F. |
| Lambda Carrageenan | 250° F. | 1 | hr | Na—PO4-Lambda |
| | | | | Carrageenan 250 F. |
| Kappa Carrageenan | 250° F. | 1 | hr | Na—PO4-Kappa |
| | | | | Carrageenan 250 F. |
| Alginate | 250° F. | 1 | hr | Na—PO4—Na- |
| | | | | Alginate 250 F. |
| Gellan Gum | 250° F. | 1 | hr | Na—PO4-Gellan Gum |
| | | | | 250 F. |
| Xanthan Gum | 250° F. | 1 | hr | Na—PO4-Xanthan |
| | | | | Gum 250 F. |
| Hyaluronic Acid | 200° F. | 2 | hrs | Na—PO4-Hyaluronic |
| | | | | Acid 200 F. |

Additional phosphate modified polysaccharides were synthesized using Protocol 1 with varying reactant quantities. The type of polysaccharide used and selected time, temperature, and reactant variations are listed in Table 4-2

TABLE 4-2
Phosphate Modified Carbohydrates Produced Using Protocol 1

| Carbohydrate | Reactant | Heating | Heating | |
|---|---|---|---|---|
| (Carb) | Quantity/Ratios | Temperature | Time | Reference Name |
| DEAE-Dextran~10 | 0.05 g Carb + | 280° F. | 1 hr | Na—PO4-DEAE |
| kDa | 0.05 g Na—PO4 | | | Dextran 280 F. |
| Cationic Guar | 0.1 g Carb + | 300° F. | 2 hr | Na—PO4-Cationic |
| Gum (GuarCat) | 0.05 g Na—PO4 | | | Guar 300 F. |
| Beta- | 0.2 g Carb + | 300° F. | 2 hr | Na—PO4-Beta |
| Cyclodextrin | 0.1 g Na—PO4 | | | Cyclodextrin |
| Gamma- | 0.2 g Carb + | 300° F. | 2 hr | Na—PO4-Gamma |
| Cyclodextrin | 0.1 g Na—PO4 | | | Cyclodextrin |

Additional cationic quaternary ammonium modified polysaccharides were synthesized using Protocol 2. The type of polysaccharide used and selected time and temperature variations are listed in Table 5-1.

TABLE 5-1
Cationic Quaternary Ammonium Modified
Carbohydrates Produced Using Protocol 2

| | Heating | Heating | | |
|---|---|---|---|---|
| Carbohydrate | Temperature | Time | | Reference Name |
| CMC | 200° F. | 1 | hr | Cationic-CMC 200 F. |
| Fucoidan | 200° F. | 0.5 | hr | Cationic-Fucoidan |
| | | | | 200 F. |
| Polyquaternium-10 | 200° F. | 1 | hr | Cationic-Polyquat- |
| | | | | 10 200 F. |
| Lambda | 200° F. | 1 | hr | Cationic-Lambda |
| Carrageenan | | | | Carrageenan 200 F. |
| Kappa | 200° F. | 1 | hr | Cationic-Kappa |
| Carrageenan | | | | Carrageenan 200 F. |
| Alginate | 200° F. | 2 | hr | Cationic-Alginate |
| | | | | 200 F. |
| Gellan Gum | 200° F. | 2 | hr | Cationic-Gellan Gum |
| | | | | 200 F. |
| Xanthan Gum | 200° F. | 1 | hr | Cationic-Xanthan Gum |
| | | | | 200 F. |
| Hyaluronic Acid | 200° F. | 1 | hrs | Cationic-Hyaluronic |
| | | | | Acid 200 F. |

Additional cationic quaternary ammonium modified polysaccharides were synthesized using Protocol 2 with varying reactant quantities. The type of polysaccharide used and selected, time, temperature, and reactant variations are listed in Table 5-2.

TABLE 5-2
Cationic Quaternary Ammonium Modified
Carbohydrates Produced Using Protocol 2

| Carbohydrate | Reactant | Heating | Heating | |
|---|---|---|---|---|
| (Carb) | Quantity/Ratios | Temperature | Time | Reference Name |
| Beta- | 0.2 g Carb + | 200° F. | 1 hr | Cationic Beta |
| Cyclodextrin | 0.1 g Betaine-HCl | | | Cyclodextrin |
| | 2.5 uL Lactic Acid | | | |
| Gamma- | 0.2 g Carb + | 200° F. | 1 hr | Cationic Gamma |
| Cyclodextrin | 0.1 g Betaine-HCl | | | Cyclodextrin |
| | 2.5 uL Lactic Acid | | | |

Additional Dual Phosphate and Cationic Quaternary Ammonium Modified Polysaccharides were synthesized using Protocol 4 with varying reactant quantities. The type of polysaccharide used and selected time, temperature, and reactant variations are listed in Table 5-3.

TABLE 5-3
Dual Phosphate and Cationic Quaternary Ammonium
Modified Carbohydrates Produced Using Protocol 4

| Carbohydrate | Reactant | Heating | Heating | |
|---|---|---|---|---|
| (Carb) | Quantity/Ratios | Temperature | Time | Reference Name |
| Na—PO4-Beta | 0.2 g Carb + | 200° F. | 1 hr | Dual PO4-Cationic |
| Cyclodextrin | 0.1 g Betaine-HCl | | | Beta Cyclodextrin |
| | 2.5 uL Lactic Acid | | | |
| Na—PO4-Gamma | 0.2 g Carb + | 200° F. | 1 hr | Dual PO4-Cationic |
| Cyclodextrin | 0.1 g Betaine-HCl | | | Gamma Cyclodextrin |
| | 2.5 uL Lactic Acid | | | |

### Example 5-B

### Production of Crosslinked Modified Starch/Carbohydrates—Herein Referred to as Protocol 5

Crosslinking of modified starch/carbohydrates was performed according to the following reference: L. Passauer, F. Liebner, K. Fischer, Synthesis and Properties of Novel Hydrogels from Cross-linked Starch Phosphates, Macromolecular Symposia. 244 (2006) 180-193.

### Briefly:

Crosslinked modified starch was synthesized by mixing 0.125 g of Na—PO4-Starch 250F in 500 μL water supplemented with 1 mM citric acid (Sigma-Aldrich, Product #251275). The modified starch citric acid was gently mixed to create a uniform slurry. The slurry was then dried at 120° F.-140° F. Following drying, the mixture was heated to about 300° F. for 1 hr. Following heating, the dried cake was washed 3 times with 2 mL of ethanol and allowed to dry overnight. The following day, the cake was resuspended in molecular biology grade water at the desired concentration (typically 500-250 mg/mL) and the pH of the suspension was adjusted to 7 using NaOH. This solution was used in future RNA stability tests and referred to as PO4-Starch-X-Link-Citric Acid.

Protocol 5 was used to produce several variations of crosslinked modified carbohydrates using different concentrations of citric acid as listed in Table 6. Depending on the type of carbohydrate used, the time and temperature of heating was selected as shown in Table 6. In some cases, both a phosphate modified carbohydrate and a cationic quaternary ammonium modified carbohydrate were crosslinked together.

TABLE 6
Crosslinked Modified Carbohydrates Produced Using Protocol 5

| | Crosslinker | Heating | Heating | |
|---|---|---|---|---|
| Carbohydrate | Concentration | Temperature | Time | Reference Name |
| Na—PO4 Sorbitol | 100 mM Citric | 300° F. | 2 hr | PO4-Sorbitol-X- |
| 280 F. | Acid | | | Link-Citric Acid |
| Na—PO4 Starch 250 F. | 1 mM Citric Acid | 300° F. | 1 hr | PO4-Starch-X- |
| | | | | Link-Citric Acid |
| Na—PO4 Zulkowsky | 10 mM Citric | 250° F. | 1 hr | PO4-Zulkowsky |
| Starch 220 F. | Acid | | | Starch-X-Link- |
| | | | | Citric Acid |
| Cationic-Sorbitol | 100 mM Citric | 300° F. | 1 hr | Cationic-Sorbitol- |
| 300 F. | Acid | | | X-Link-Citric Acid |
| Cationic-Starch 200 F. | 1 mM Citric Acid | 250° F. | 1 hr | Cationic-Starch-X- |
| | | | | Link-Citric Acid |
| Cationic-Zulkowsky | 10 mM Citric | 250° F. | 1 hr | Cationic- |
| Starch 200 F. | Acid | | | Zulkowsky Starch - |
| | | | | X-Link-Citric Acid |
| Na—PO4 Sorbitol | 100 mM Citric | 300° F. | 2 hr | PO4-Cationic- |
| 280 F. + Cationic- | Acid | | | Sorbitol-X-Link- |
| Sorbitol 300 F. | | | | Citric Acid |
| Na—PO4 Starch 250 | 1 mM Citric Acid | 250° F. | 1 hr | PO4-Cationic- |
| F. + Cationic-Starch | | | | Starch-X-Link- |
| 200 F. | | | | Citric Acid |
| Na—PO4 Zulkowsky | 10 mM Citric | 250° F. | 1 hr | PO4-Cationic- |
| Starch 220 F. + | Acid | | | Zulkowsky Starch- |
| Cationic-Zulkowsky | | | | X-Link-Citric Acid |
| Starch 200 F. | | | | |

Protocol 5 was used to produce several variations of crosslinked modified carbohydrates substituting selected concentrations of malic acid for citric acid at the selected heating times and heating temperatures as listed in Table 7.

TABLE 7
Crosslinked Modified Carbohydrates Produced Using Protocol 5

| | Crosslinker | Heating | Heating | |
|---|---|---|---|---|
| Carbohydrate | Concentration | Temperature | Time | Reference Name |
| Na—PO4 Sorbitol 280 F. | 100 mM Malic | 300° F. | 2 hr | PO4-Sorbitol-X- |
| | Acid | | | Link-Malic Acid |
| Na—PO4 Starch 250 F. | 1 mM Malic Acid | 300° F. | 1 hr | PO4-Starch-X- |
| | | | | Link-Malic Acid |
| Na—PO4 Zulkowsky | 10 mM Malic Acid | 250° F. | 1 hr | PO4-Zulkowsky |
| Starch 220 F. | | | | Starch-X-Link- |
| | | | | Malic Acid |
| Cationic-Sorbitol 300 F. | 100 mM Malic | 300° F. | 1 hr | Cationic-Sorbitol- |
| | Acid | | | X-Link-Malic Acid |
| Cationic-Starch 200 F. | 1 mM Malic Acid | 250° F. | 1 hr | Cationic-Starch-X- |
| | | | | Link-Malic Acid |
| Cationic-Zulkowsky | 10 mM Malic Acid | 250° F. | 1 hr | Cationic- |
| Starch 200 F. | | | | Zulkowsky Starch- |
| | | | | X-Link-Malic Acid |
| Na—PO4 Sorbitol 280 F. + | 100 mM Malic | 300° F. | 2 hr | PO4-Cationic- |
| Cationic-Sorbitol 300 F. | Acid | | | Sorbitol-X-Link- |
| | | | | Malic Acid |
| Na—PO4 Starch 250 F. + | 1 mM Malic Acid | 250° F. | 1 hr | PO4-Cationic- |
| Cationic-Starch 200 F. | | | | Starch-X-Link- |
| | | | | Malic Acid |
| Na—PO4 Zulkowsky | 10 mM Malic Acid | 250° F. | 1 hr | PO4-Cationic- |
| Starch 220 F. + Cationic- | | | | Zulkowsky Starch- |
| Zulkowsky Starch 200 F. | | | | X-Link-Malic Acid |

### Example 6

### In Vitro Transcription and Denaturing Agarose Gel Electrophoresis

The RNA was synthesized by in vitro transcription from a linear DNA construct (Twist Bioscience, South San Francisco, CA) with an upstream T7 RNA Polymerase promoter followed by the coding sequence for gene of interest. In vitro transcription was performed using the HiScribe T7 Quick High Yield RNA Synthesis Kit (New England Biolabs, Ipswich, MA; Product #E2050) according to the manufacturer's instructions. Briefly, 2.5 μg template DNA was mixed with 25 μL NTP buffer mix and 5 μL T7 RNA polymerase mix. The entire reaction volume was brought to 50 μL with molecular biology grade H₂O and incubated in a thermal cycler at 37° C. for about 2 hrs.

Following in vitro transcription, the RNA was purified using a Monarch RNA Cleanup Kit (New England Biolabs, Ipswich, MA; Product #T2050) according to the manufacturer's instructions. Briefly, 1 spin column was used for each 50 μL reaction. Following binding of the RNA to the spin column, 2 washes of 500 μL were performed and the RNA was eluted with 100-150 μL of molecular biology grade H₂O. The purified RNA was then stored at −80° C.

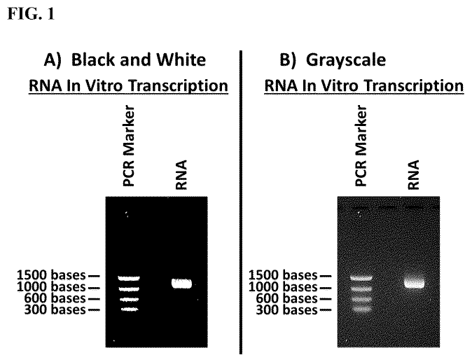

The in vitro transcribed and purified RNA was analyzed by denaturing agarose gel electrophoresis. Briefly, about 5 μg RNA was diluted 1:1 with 2×RNA loading dye (New England Biolabs, Ipswich, MA; Product #B0363) and heated to about 70° C. for about 2-4 minutes to denature the RNA. The final concentration of RNA and loading dye was about: 5 μg RNA, 47.5% formamide, 0.01% SDS, 0.01% bromophenol blue, 0.005% xylene cyanol and 0.5 mM EDTA. The RNA was run on a 1.5% agarose gel made with 1× Tris Acetate EDTA (1×TAE buffer) (Tris 40 mM, Acetic acid 20 mM, EDTA 1 mM, pH 8.0) supplemented with 0.06% sodium hypochlorite (NaClO) to prevent renaturing and degradation of the RNA during electrophoresis. The running buffer also contained 1×TAE buffer supplemented with 0.06% sodium hypochlorite. RNA was visualized using SmartGlow fluorescent nucleic acid prestain (Accuris Instruments, Edison, NJ; Product #E4500-PS) according to the manufacturer's instructions. A double stranded DNA PCR Marker (New England Biolabs, Ipswich, MA; Product #N3234) was used to estimate the apparent molecular weight of the RNA during electrophoresis. Denaturing agarose gel electrophoresis was carried out for about 60-90 min at 80V. The in vitro transcribed and purified RNA analyzed by denaturing agarose gel electrophoresis is shown in FIG. 1.

RNA concentration was measured by absorbance of the purified RNA at 260 nm using a Nanodrop ND-1000 (Thermo Fisher Scientific, Waltham, MA). Typical RNA concentration following in vitro transcription and purification was about 2-5 mg/mL.

### Example 7

### Stability of RNA at Different Temperatures

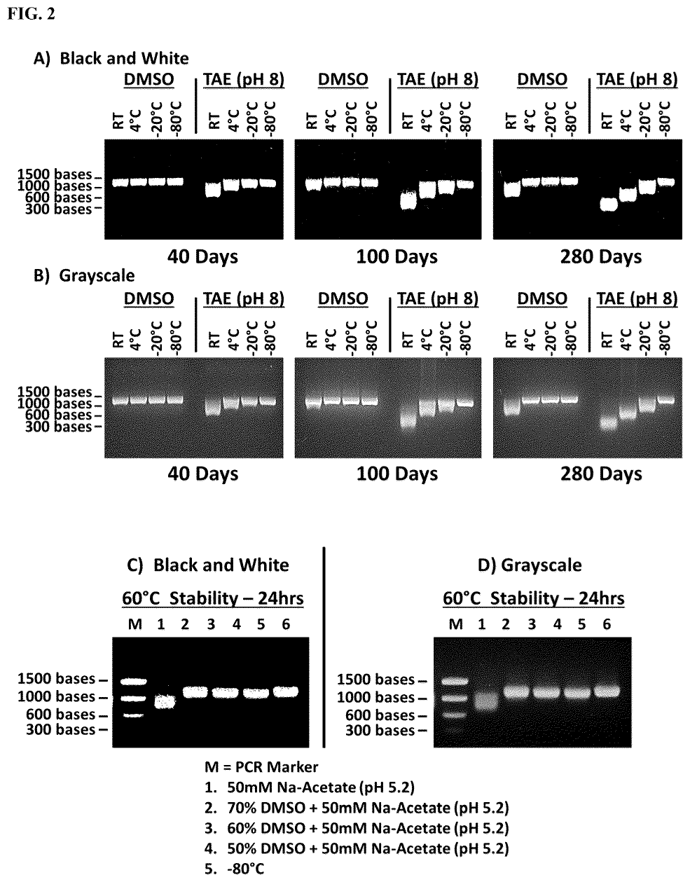

In vitro transcribed RNA was diluted at a ratio of about 1:10 (about 400-500 μg/mL) in either DMSO (Sigma-Aldrich, St. Louis, MO; Product #D8418) or 1× Tris Acetate EDTA (TAE) (pH 8) (Bioland Scientific LLC, Paramount, CA; Product #TAE01). The final concentration of DMSO was about 90% DMSO. The final concentration of TAE was about Tris 40 mM, Acetic acid 20 mM, EDTA 1 mM, pH 8.0. Following dilution of the RNA in either DMSO or TAE, the samples were then stored at 4 different temperatures: room temperature (RT) (about 20 to 30° C.), about 4° C., about −20° C., and about −80° C. Samples were then analyzed by denaturing agarose gel electrophoresis as described above at selected timepoints to measure RNA degradation and the stability of the RNA samples stored in either DMSO or TAE. During storage, 10 μL of each sample was analyzed at selected timepoints by agarose gel electrophoresis to measure RNA degradation and the stability of the RNA samples stored at each temperature in either DMSO or TAE. FIGS. 2A and B shows the agarose gel electrophoresis of each RNA sample following storage of the RNA for about 40 days to about 280 days at different temperatures.

The RNA sample stored in DMSO displays increased stability and reduced rate of degradation of the RNA sample as shown by agarose gel electrophoresis. The RNA sample stored in TAE begins to show significant degradation at room temperature after about 40 days as indicated by the decreasing apparent molecular weight compared to the −80° C. RNA sample. While the RNA sample stored in DMSO does not show significant signs of degradation until about 100 days at room temperature. Furthermore, the RNA sample stored in TAE begins to show significant signs of degradation following about 40 days at 4° C. While the RNA sample stored in DMSO does not show significant signs of degradation up to about 280 days at 4° C. In addition, the RNA sample stored in TAE begins to show significant signs of degradation following about 100 days at −20° C. While the RNA sample stored in DMSO does not show significant signs of degradation up to about 280 days at −20° C. Following 100 days, it becomes apparent that the RNA sample stored in DMSO shows comparable and/or better stability at room temperature when compared to the RNA sample stored in TAE at 4° C. or −20° C. Furthermore, RNA stored in DMSO at 4° C. still displays a band with the majority of the RNA running at a comparable apparent molecular weight compared to the −80° C. RNA sample following 280 days. Meanwhile, the RNA stored in TAE shows little to no band of comparable apparent molecular weight, with the majority of the RNA running at a significantly lower apparent molecular weight, compared to the −80° C. RNA sample following 280 days at 4° C. or −20° C.

### Accelerated Stability Testing

Accelerated stability testing is known in the art, where a sample, such as RNA as a non-limiting example, is incubated at elevated temperature to increase the rate of degradation and evaluate stability over a selected duration of time. The following examples compare RNA degradation in different storage environments containing different substances or combinations of substances stored at elevated temperatures and sampled at selected timepoints.

Accelerated stability testing to evaluate RNA stability in several of the following examples was carried out as follows:

Several of the following examples show the results of denaturing agarose gel electrophoresis comparing accelerated RNA stability in different RNA storage environments comprising different substances, either alone or in combination.

In vitro transcribed RNA was diluted at a ratio of about 1:10 (about 100-300 μg/mL) in different compositions. A control sample, where RNA was diluted in a composition containing either a buffer only control or a control without one or more substance tested was used. The RNA stability of these control samples was then compared to RNA diluted in compositions containing different substances or combinations of multiple substances.

Following dilution of the RNA into each respective RNA storage environment, samples were stored in a thermal cycler at selected elevated temperatures for selected periods of time as described below. During storage at elevated temperature, samples were analyzed at selected timepoints by denaturing agarose gel electrophoresis to measure RNA degradation and the stability of the RNA samples in each RNA storage environment.

Denaturing agarose gel electrophoresis was performed as described in Example 6 above. Briefly, following storage of the RNA in each selected RNA storage environment at the selected time and temperature, the RNA samples were analyzed on a 1.5% agarose gel made with 1×TAE buffer supplemented with about 0.03-0.04% sodium hypochlorite (NaClO) to prevent renaturing and degradation of the RNA during electrophoresis. The running buffer also contained 1×TAE buffer supplemented with about 0.03-0.04% sodium hypochlorite. Prior to running the RNA samples on the agarose gel, samples were mixed at a 1:1 ratio with 2×RNA loading dye. The RNA samples were then heated to about 70° C. for about 2-4 minutes to denature the RNA prior to running. RNA was visualized using a fluorescent nucleic acid dye according to the manufacturer's instructions. A double stranded DNA PCR Marker (New England Biolabs, Ipswich, MA; Product #N3234) was used to estimate the apparent molecular weight of the RNA during electrophoresis. Denaturing agarose gel electrophoresis was carried out for about 60-90 min at 80V.

A control sample (typically a buffer only control) was compared to the subsequent samples with at least one or more additional substance not present in the control sample. An RNA sample stored at −80° C. (typically run in the final lane) was used to compare the size of the RNA stored at elevated temperatures to a full-length RNA sample stored at −80° C.

Following analysis by agarose gel electrophoresis, RNA degradation was measured by comparing the relative decrease in apparent molecular weight of the control sample to the samples tested, while also using the PCR Marker, and −80° C. full length RNA sample as a guide for measuring changes in apparent molecular weight.

An example agarose used for denaturing agarose gel electrophoresis is Ultra-Pure, LE agarose such as Apex General Purpose LE Agarose, Ultra-Pure (Apex Bioresearch Products, Ordered from Genesee Scientific, El Cajon, CA, Cat #20-101). An example 2×RNA loading dye used for denaturing agarose gel electrophoresis is 2×RNA loading dye (New England Biolabs; Product #B0363) supplemented with 0.2% SDS. An example fluorescent dye used for visualizing RNA during denaturing agarose gel electrophoresis is SmartGlow fluorescent nucleic acid prestain (Accuris Instruments, Edison, NJ; Product #E4500-PS).

Agarose gel electrophoresis is a common method known in the art. One of ordinary skill in the art would appreciate that certain parameters may occasionally be adjusted to help improve analysis by gel electrophoresis when necessary, such as increasing the amount or concentration of RNA loaded onto a gel, increasing the amount of fluorescent dye used to detect RNA, or diluting or buffer exchanging samples with high viscosity or background (e.g. background absorbance or background fluorescence) are common methods that may be employed.

In the following examples the substances and compounds tested were used at about neutral pH (e.g. 7) or adjusted to a pH of about 7 using NaOH or HCl unless stated otherwise.

Common abbreviations in following examples: hexametaphosphate (HMP), trimetaphosphate (TMP), Uridine-5′-Monophosphate (Uridine 5′-PO₄), Guanosine-5′-Monophosphate (Guanosine 5′-PO₄), Inosine-5′-Monophosphate (Inosine 5′-PO₄), Xanthosine 5′-Monophosphate (Xanthosine 5′-PO₄), 2′-Deoxyguanosine-5′-Monophosphate (2′-Deoxyguanosine-5′-PO₄), Thymidine-5′-Monophosphate (2′-Deoxythymidine-5′-PO₄), 2′-Deoxyuridine-5′-Monophosphate (2′-Deoxyuridine-5′-PO₄), Cyclic Guanosine-Monophosphate (Cyclic-GMP), Phosphate (PO₄).

### Example 8

### Accelerated RNA Stability Testing at 60° C.

In vitro transcribed RNA was diluted at a ratio of about 1:10 (about 100-300 μg/mL) in different compositions containing either 50 mM sodium acetate (pH 5.2) (Sigma-Aldrich, St. Louis, MO; Product #S7899), or a mixture of DMSO and 50 mM sodium acetate (pH 5.2) as follows:
1. 50 mM Na-Acetate (pH 5.2)
2. 70% DMSO+50 mM Na-Acetate (pH 5.2)
3. 60% DMSO+50 mM Na-Acetate (pH 5.2)
4. 50% DMSO+50 mM Na-Acetate (pH 5.2)
5. −80° C.

Following dilution of each sample in each respective RNA storage environment, samples were stored in a thermal cycler at 60° C. for about 24 hours and then analyzed by denaturing agarose gel electrophoresis as shown in FIGS. 2 C and D.

### Example 9

### Accelerated RNA Stability Testing at 60° C.

In vitro transcribed RNA was diluted at a ratio of about 1:10 (about 100-300 μg/mL) in different compositions containing either 50 mM sodium acetate (pH 5.2), or a mixture of sodium trimetaphosphate (TMP) (Sigma-Aldrich, St. Louis, MO; Product #PHR2204) and 50 mM sodium acetate (pH 5.2) as follows:
1. 50 mM Na-Acetate (pH 5.2)
2. 100 mM Na-Trimetaphosphate+50 mM Na-Acetate (pH 5.2)
3. 90 mM Na-Trimetaphosphate+50 mM Na-Acetate (pH 5.2)
4. 80 mM Na-Trimetaphosphate+50 mM Na-Acetate (pH 5.2)
5. −80° C.

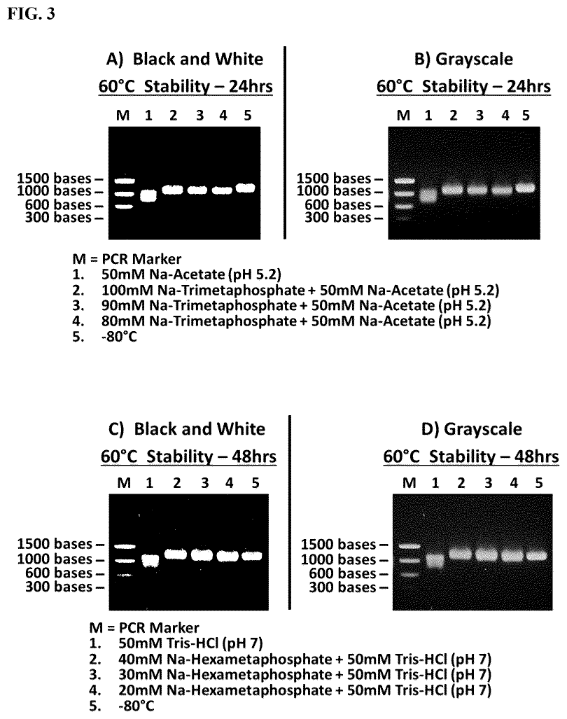

Following dilution of each sample in each respective RNA storage environment, samples were stored in a thermal cycler at 60° C. for about 24 hours and then analyzed by denaturing agarose gel electrophoresis as shown in FIGS. 3A and B.

### Example 10

### Accelerated RNA Stability Testing at 60° C.

In vitro transcribed RNA was diluted at a ratio of about 1:10 (about 100-300 μg/mL) in different compositions containing either 50 mM Tris-HCl (pH 7) (Bioland Scientific LLC, Paramount, CA; Product #Tris70), or a mixture of sodium hexametaphosphate (HMP) and 50 mM Tris-HCl (pH 7) as follows:
1. 50 mM Tris-HCl (pH 7)
2. 40 mM Na-Hexametaphosphate+50 mM Tris-HCl (pH 7)
3. 30 mM Na-Hexametaphosphate+50 mM Tris-HCl (pH 7)
4. 20 mM Na-Hexametaphosphate+50 mM Tris-HCl (pH 7)
5. −80° C.

Following dilution of each sample in each respective RNA storage environment, samples were stored in a thermal cycler at 60° C. for about 48 hours and then analyzed by denaturing agarose gel electrophoresis as shown in FIGS. 3 C and D.

### Example 11

### Accelerated RNA Stability Testing at 60° C.

In vitro transcribed RNA was diluted at a ratio of about 1:10 (about 100-300 μg/mL) in different compositions containing either 50 mM sodium acetate (pH 5.2), or a mixture of hexylene glycol (Sigma-Aldrich, St. Louis, MO; Product #M9671) and 50 mM sodium acetate (pH 5.2) as follows:
1. 50 mM Na-Acetate (pH 5.2)
2. 30% Hexylene Glycol+50 mM Na-Acetate (pH 5.2)
3. 27.5% Hexylene Glycol+50 mM Na-Acetate (pH 5.2)
4. 25% Hexylene Glycol+50 mM Na-Acetate (pH 5.2)
5. 22.5% Hexylene Glycol+50 mM Na-Acetate (pH 5.2)
6. −80° C.

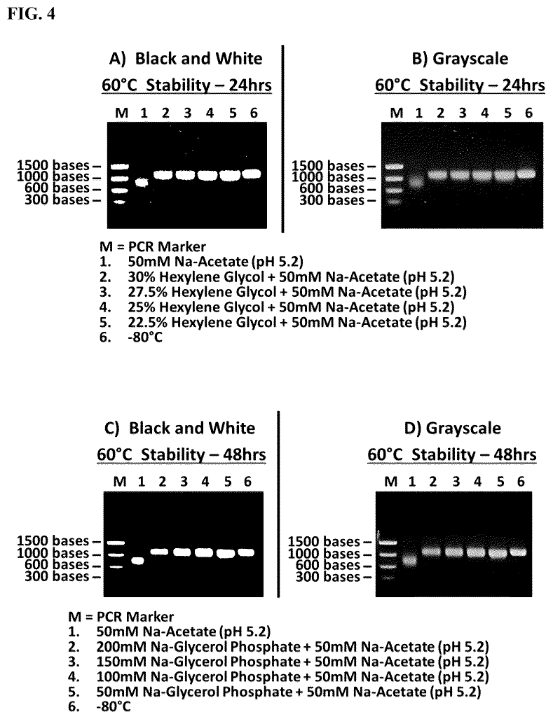

Following dilution of each sample in each respective RNA storage environment, samples were stored in a thermal cycler at 60° C. for about 24 hours and then analyzed by denaturing agarose gel electrophoresis as shown in FIGS. 4A and B.

### Example 12

### Accelerated RNA Stability Testing at 60° C.

In vitro transcribed RNA was diluted at a ratio of about 1:10 (about 100-300 μg/mL) in different compositions containing either 50 mM sodium acetate (pH 5.2), or a mixture of glycerol phosphate disodium salt (Sigma-Aldrich, St. Louis, MO; Product #G6501) and 50 mM sodium acetate (pH 5.2) as follows:
1. 50 mM Na-Acetate (pH 5.2)
2. 200 mM Na-Glycerol Phosphate+50 mM Na-Acetate (pH 5.2)
3. 150 mM Na-Glycerol Phosphate+50 mM Na-Acetate (pH 5.2)
4. 100 mM Na-Glycerol Phosphate+50 mM Na-Acetate (pH 5.2)
5. 50 mM Na-Glycerol Phosphate+50 mM Na-Acetate (pH 5.2)
6. −80° C.

Following dilution of each sample in each respective RNA storage environment, samples were stored in a thermal cycler at 60° C. for about 48 hours and then analyzed by denaturing agarose gel electrophoresis as shown in FIGS. 4 C and D.

### Example 13

### Accelerated RNA Stability Testing at 60° C.

In vitro transcribed RNA was diluted at a ratio of about 1:10 (about 100-300 μg/mL) in different compositions containing either 50 mM sodium acetate (pH 5.2), or a mixture of 1-butyl-1-methylpyrrolidinium bromide (Sigma-Aldrich, St. Louis, MO; Product #04275), benzyltriethylammonium chloride (Sigma-Aldrich, St. Louis, MO; Product #146552), or N,N-dimethylphenethylamine (Sigma-Aldrich, St. Louis, MO; Product #523801) and 50 mM sodium acetate (pH 5.2) as follows:
1. 50 mM Na-Acetate (pH 5.2)
2. 750 mM 1-Buty-1-Methylpyrrolidinium-Br+50 mM Na-Acetate (pH 5.2)
3. 500 mM Benzyltriethylammonium-Cl+50 mM Na-Acetate (pH 5.2)
4. 100 mM N,N-Dimethylphenethylamine+50 mM Na-Acetate (pH 5.2)
5. −80° C.

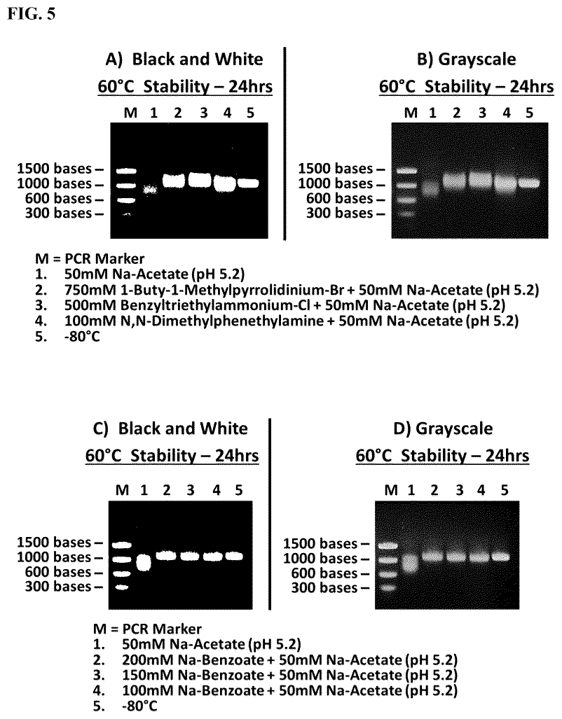

The N,N-dimethylphenethylamine sample dilution was made fresh from the stock solution immediately prior to mixing with RNA. Following dilution of each sample in each respective RNA storage environment, samples were stored in a thermal cycler at 60° C. for about 24 hours and then analyzed by denaturing agarose gel electrophoresis as shown in FIGS. 5A and B. Prior to performing gel electrophoresis, the 1-butyl-1-methylpyrrolidinium and benzyltriethylammonium samples were incubated with 2.5 mM ˜8 kDa PAA sodium salt for about 1 hour at room temperature (about 20-30° C.) to improve analysis by gel electrophoresis.

### Example 14

### Accelerated RNA Stability Testing at 60° C.

In vitro transcribed RNA was diluted at a ratio of about 1:10 (about 100-300 μg/mL) in different compositions containing either 50 mM sodium acetate (pH 5.2), or a mixture of sodium benzoate (Sigma-Aldrich, St. Louis, MO; Product #109169) and 50 mM sodium acetate (pH 5.2) as follows:
1. 50 mM Na-Acetate (pH 5.2)
2. 200 mM Na-Benzoate+50 mM Na-Acetate (pH 5.2)
3. 150 mM Na-Benzoate+50 mM Na-Acetate (pH 5.2)
4. 100 mM Na-Benzoate+50 mM Na-Acetate (pH 5.2)
5. −80° C.

Following dilution of each sample in each respective RNA storage environment, samples were stored in a thermal cycler at 60° C. for about 24 hours and then analyzed by denaturing agarose gel electrophoresis as shown in FIGS. 5 C and D.

### Example 15

### Accelerated RNA Stability Testing at 60° C.

In vitro transcribed RNA was diluted at a ratio of about 1:10 (about 100-300 μg/mL) in different compositions containing either 50 mM Tris-HCl (pH 7), or a mixture of sodium benzoate with 1M trimethylglycine (TMG) (Sigma-Aldrich, St. Louis, MO; Product #B0300) and 50 mM Tris-HCl (pH 7) as follows:
1. 50 mM Tris-HCl (pH 7)
2. 300 mM Na-Benzoate+1M TMG+50 mM Tris-HCl (pH 7)
3. 250 mM Na-Benzoate+1M TMG+50 mM Tris-HCl (pH 7)
4. 200 mM Na-Benzoate+1M TMG+50 mM Tris-HCl (pH 7)
5. −80° C.

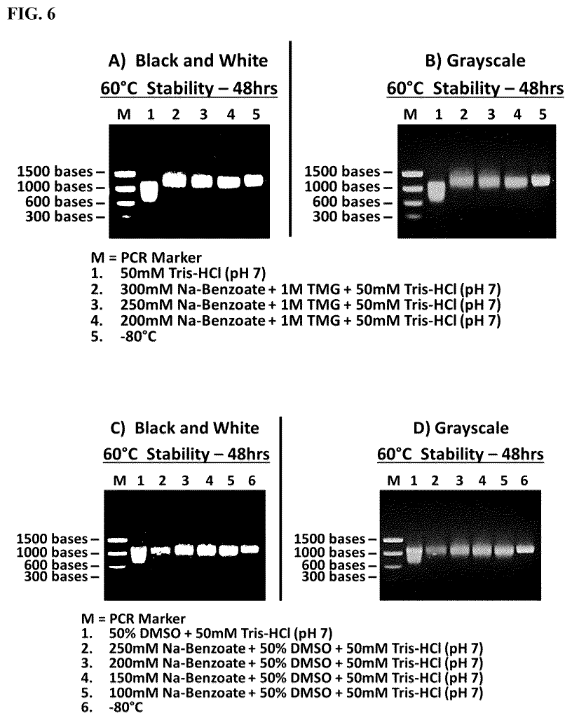

Following dilution of each sample in each respective RNA storage environment, samples were stored in a thermal cycler at 60° C. for about 48 hours and then analyzed by denaturing agarose gel electrophoresis as shown in FIGS. 6A and B.

### Example 16

### Accelerated RNA Stability Testing at 60° C.

In vitro transcribed RNA was diluted at a ratio of about 1:10 (about 100-300 μg/mL) in different compositions containing either 50% DMSO with 50 mM Tris-HCl (pH 7), or a mixture of sodium benzoate with 50% DMSO and 50 mM Tris-HCl (pH 7) as follows:
1. 50% DMSO+50 mM Tris-HCl (pH 7)
2. 250 mM Na-Benzoate+50% DMSO+50 mM Tris-HCl (pH 7)
3. 200 mM Na-Benzoate+50% DMSO+50 mM Tris-HCl (pH 7)
4. 150 mM Na-Benzoate+50% DMSO+50 mM Tris-HCl (pH 7)
5. 100 mM Na-Benzoate+50% DMSO+50 mM Tris-HCl (pH 7)
6. −80° C.

Following dilution of each sample in each respective RNA storage environment, samples were stored in a thermal cycler at 60° C. for about 48 hours and then analyzed by denaturing agarose gel electrophoresis as shown in FIGS. 6 C and D.

### Example 17

### Accelerated RNA Stability Testing at 60° C.

In vitro transcribed RNA was diluted at a ratio of about 1:10 (about 100-300 μg/mL) in different compositions containing either 50 mM sodium acetate (pH 5.2), or varying concentrations of trimethyloctylammonium bromide (Sigma-Aldrich, St. Louis, MO; Product #75091) and 50 mM sodium acetate (pH 5.2) as follows:
1. 50 mM Na-Acetate (pH 5.2)
2. 70 mM Trimethyloctylammonium-Br+50 mM Na-Acetate (pH 5.2)
3. 60 mM Trimethyloctylammonium-Br+50 mM Na-Acetate (pH 5.2)
4. 50 mM Trimethyloctylammonium-Br+50 mM Na-Acetate (pH 5.2)
5. 40 mM Trimethyloctylammonium-Br+50 mM Na-Acetate (pH 5.2)
6. −80° C.

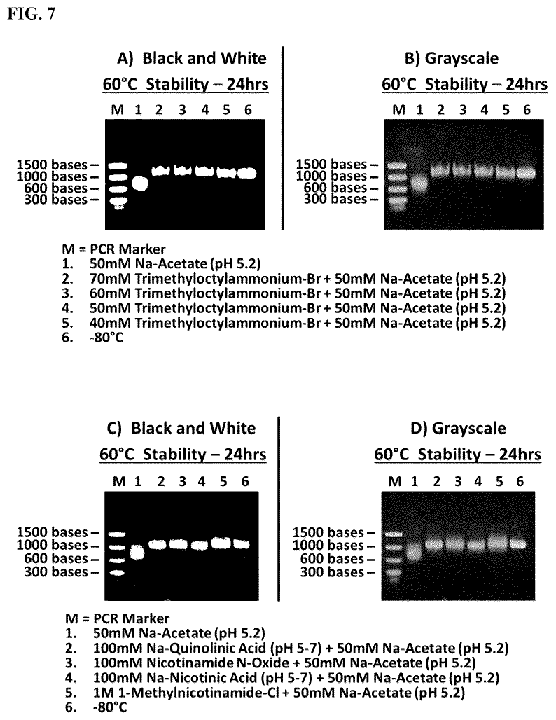

Following dilution of each sample in each respective RNA storage environment, samples were stored in a thermal cycler at 60° C. for about 24 hours and then analyzed by denaturing agarose gel electrophoresis as shown in FIGS. 7A and B. Prior to performing gel electrophoresis, the trimethyloctylammonium samples were diluted 1.5× with molecular biology H₂O (10 μL H₂O added to a 20 μL sample) and then incubated with 2.5 mM ˜8 kDa PAA sodium salt for about 1 hour at room temperature (about 20-30° C.) to improve analysis by gel electrophoresis.

### Example 18

### Accelerated RNA Stability Testing at 60° C.

In vitro transcribed RNA was diluted at a ratio of about 1:10 (about 100-300 μg/mL) in different compositions containing either 50 mM sodium acetate (pH 5.2), or a mixture of quinolinic acid (Cayman Chemical, Ann Arbor, MI; Product #14941), nicotinamide N-oxide (Cayman Chemical, Ann Arbor, MI; Product #28441), nicotinic acid (Sigma-Aldrich, St. Louis, MO; Product #N4126), or 1-methylnicotinamide chloride (Cayman Chemical, Ann Arbor, MI; Product #16604) and 50 mM sodium acetate (pH 5.2) as follows:
1. 50 mM Na-Acetate (pH 5.2)
2. 100 mM Na-Quinolinic Acid (pH 5-7)+50 mM Na-Acetate (pH 5.2)
3. 100 mM Nicotinamide N-Oxide+50 mM Na-Acetate (pH 5.2)
4. 100 mM Na-Nicotinic Acid (pH 5-7)+50 mM Na-Acetate (pH 5.2)
5. 1M 1-Methylnicotinamide-Cl+50 mM Na-Acetate (pH 5.2)
6. −80° C.

The quinolinic acid and nicotinic acid stocks were adjusted to a pH of about 5-7 using NaOH. The nicotinamide N-oxide stock pH was adjusted using NaOH to facilitate dissolving in H₂O and then subsequently adjusted to a pH of about 5-7 using HCl once dissolved. Following dilution of each sample in each respective RNA storage environment, samples were stored in a thermal cycler at 60° C. for about 24 hours and then analyzed by denaturing agarose gel electrophoresis as shown in FIGS. 7 C and D. Prior to performing gel electrophoresis, the nicotinamide N-oxide and 1-methylnicotinamide samples were incubated with 1 mM ˜8 kDa PAA sodium salt about 1 hour at room temperature (about 20-30° C.) to improve analysis by gel electrophoresis.

### Example 19

### Accelerated RNA Stability Testing at 60° C.

In vitro transcribed RNA was diluted at a ratio of about 1:10 (about 100-300 μg/mL) in different compositions containing either 5 mM sodium phosphate (pH 8) (Sigma-Aldrich, St. Louis, MO; Product #94046 & 74092) and 10 mM sodium acetate (pH 7) (Sigma-Aldrich, St. Louis, MO; Product #S2404), or a mixture of ectoine (Sigma-Aldrich, St. Louis, MO; Product #81619), L-proline (Sigma-Aldrich, St. Louis, MO; Product #81709), glycine (Sigma-Aldrich, St. Louis, MO; Product #50046), or taurine (Sigma-Aldrich, St. Louis, MO; Product #T0625), and 5 mM sodium phosphate (pH 8) and 10 mM sodium acetate (pH 7) as follows:
1. 5 mM Na-Phosphate (pH 8)+10 mM Na-Acetate (pH 7)
2. 2M Ectoine+5 mM Na-Phosphate (pH 8)+10 mM Na-Acetate (pH 7)
3. 500 mM Proline+5 mM Na-Phosphate (pH 8)+10 mM Na-Acetate (pH 7)
4. 500 mM Glycine+5 mM Na-Phosphate (pH 8)+10 mM Na-Acetate (pH 7)
5. 250 mM Taurine+5 mM Na-Phosphate (pH 8)+10 mM Na-Acetate (pH 7)
6. −80° C.

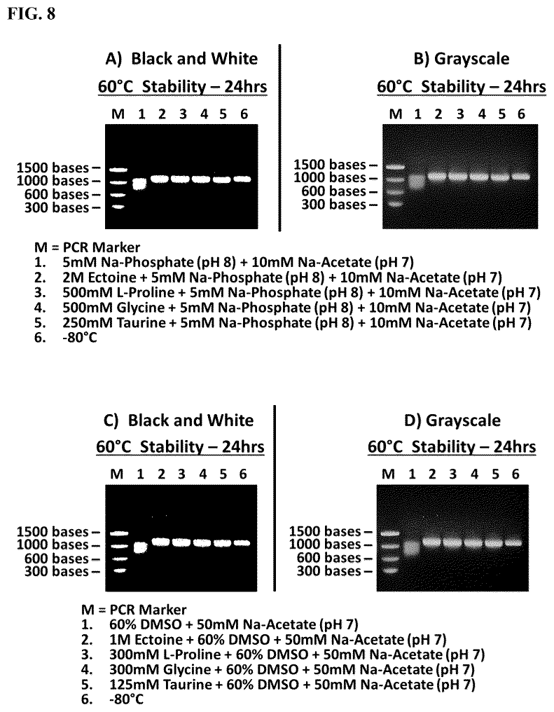

Following dilution of each sample in each respective RNA storage environment, samples were stored in a thermal cycler at 60° C. for about 24 hours and then analyzed by denaturing agarose gel electrophoresis as shown in FIGS. 8A and B.

### Example 20

### Accelerated RNA Stability Testing at 60° C.

In vitro transcribed RNA was diluted at a ratio of about 1:10 (about 100-300 μg/mL) in different compositions containing either 60% DMSO with 50 mM sodium acetate (pH 7), or a mixture of ectoine, L-proline, glycine, or taurine, and 60% DMSO with 50 mM sodium acetate (pH 7) as follows:
1. 60% DMSO+50 mM Na-Acetate (pH 7)
2. 1M Ectoine+60% DMSO+50 mM Na-Acetate (pH 7)
3. 300 mM Proline+60% DMSO+50 mM Na-Acetate (pH 7)
4. 300 mM Glycine+60% DMSO+50 mM Na-Acetate (pH 7)
5. 125 mM Taurine+60% DMSO+50 mM Na-Acetate (pH 7)
6. −80° C.

Following dilution of each sample in each respective RNA storage environment, samples were stored in a thermal cycler at 60° C. for about 24 hours and then analyzed by denaturing agarose gel electrophoresis as shown in FIGS. 8 C and D.

### Example 21

### Accelerated RNA Stability Testing at 60° C.

In vitro transcribed RNA was diluted at a ratio of about 1:10 (about 100-300 μg/mL) in different compositions containing either TAE (Tris 40 mM, Acetic acid 20 mM, EDTA 1 mM) (pH 8), or a mixture of dimethylsulfoniopropionate (DMSP) (Sigma-Aldrich, St. Louis, MO; Product #80828) with TAE (pH 8) as follows:
1. TAE (pH 8)
2. 700 mM DMSP+TAE (pH 8)
3. 600 mM DMSP+TAE (pH 8)
4. 500 mM DMSP+TAE (pH 8)
5. 400 mM DMSP+TAE (pH 8)
6. −80° C.

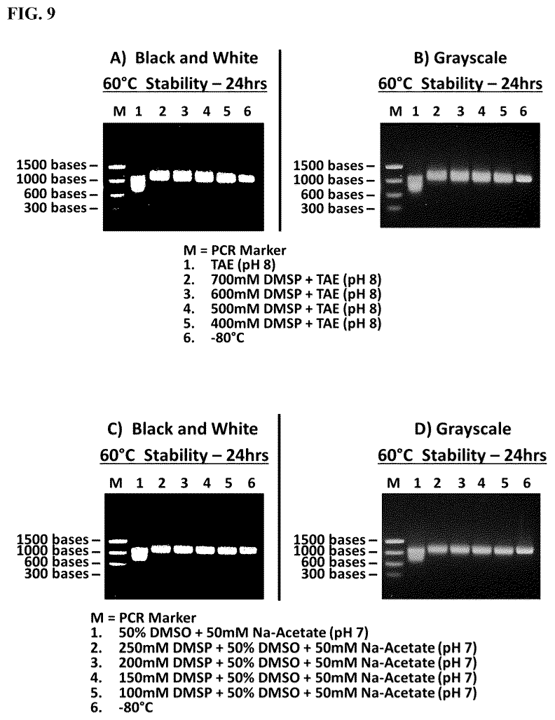

The DMSP stock was adjusted to a pH of about 7 using NaOH. Following dilution of each sample in each respective RNA storage environment, samples were stored in a thermal cycler at 60° C. for about 24 hours and then analyzed by denaturing agarose gel electrophoresis as shown in FIGS. 9A and B. Prior to performing gel electrophoresis, the DMSP samples were incubated with 1 mM ˜8 kDa PAA sodium salt about 1 hour at room temperature (about 20-30° C.) to improve analysis by gel electrophoresis.

### Example 22

Accelerated RNA Stability Testing at 60° C.

In vitro transcribed RNA was diluted at a ratio of about 1:10 (about 100-300 μg/mL) in different compositions containing either 50% DMSO with 50 mM sodium acetate (pH 7), or a mixture of DMSP and 50% DMSO with 50 mM sodium acetate (pH 7) as follows:
1. 50% DMSO+50 mM Na-Acetate (pH 7)
2. 250 mM DMSP+50% DMSO+50 mM Na-Acetate (pH 7)
3. 200 mM DMSP+50% DMSO+50 mM Na-Acetate (pH 7)
4. 150 mM DMSP+50% DMSO+50 mM Na-Acetate (pH 7)
5. 100 mM DMSP+50% DMSO+50 mM Na-Acetate (pH 7)
6. −80° C.

The DMSP stock was adjusted to a pH of about 7 using NaOH. Following dilution of each sample in each respective RNA storage environment, samples were stored in a thermal cycler at 60° C. for about 24 hours and then analyzed by denaturing agarose gel electrophoresis as shown in FIGS. 9 C and D. Prior to performing gel electrophoresis, the DMSP samples were incubated with 1 mM ˜8 kDa PAA sodium salt for about 1 hour at room temperature (about 20-30° C.) to improve analysis by gel electrophoresis.

### Example 23

### Accelerated RNA Stability Testing at 70° C.

In vitro transcribed RNA was diluted at a ratio of about 1:10 (about 100-300 μg/mL) in different compositions containing either 50 mM Tris-HCl (pH 7), or a mixture of choline chloride and 50 mM Tris-HCl (pH 7) as follows:
1. 50 mM Tris-HCl (pH 7)
2. 1M Choline-Cl+50 mM Tris-HCl (pH 7)
3. 900 mM Choline-Cl+50 mM Tris-HCl (pH 7)
4. 800 mM Choline-Cl+50 mM Tris-HCl (pH 7)
5. 700 mM Choline-Cl+50 mM Tris-HCl (pH 7)
6. −80° C.

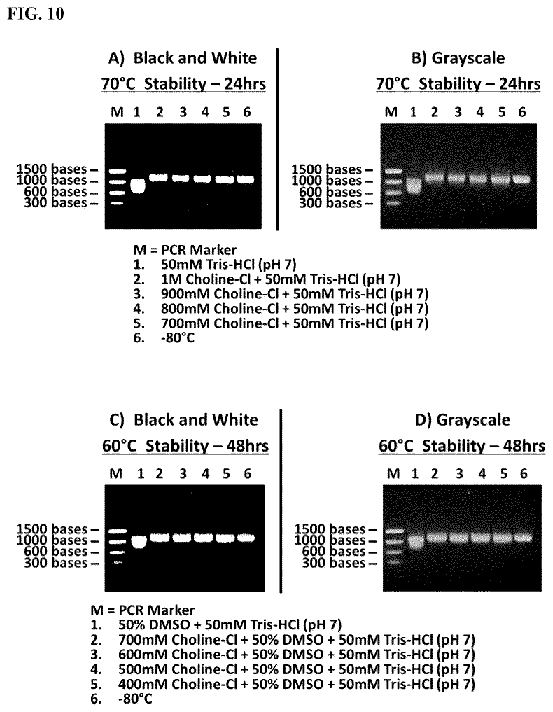

Following dilution of each sample in each respective RNA storage environment, samples were stored in a thermal cycler at 70° C. for about 24 hours and then analyzed by denaturing agarose gel electrophoresis as shown in FIGS. 10A and B.

### Example 24

### Accelerated RNA Stability Testing at 60° C.

In vitro transcribed RNA was diluted at a ratio of about 1:10 (about 100-300 μg/mL) in different compositions containing either 50% DMSO with 50 mM Tris-HCl (pH 7), or a mixture of choline chloride and 50% DMSO with 50 mM Tris-HCl (pH 7) as follows:
1. 50% DMSO+50 mM Tris-HCl (pH 7)
2. 700 mM Choline-Cl+50% DMSO+50 mM Tris-HCl (pH 7)
3. 600 mM Choline-Cl+50% DMSO+50 mM Tris-HCl (pH 7)
4. 500 mM Choline-Cl+50% DMSO+50 mM Tris-HCl (pH 7)
5. 400 mM Choline-Cl+50% DMSO+50 mM Tris-HCl (pH 7)
6. −80° C.

Following dilution of each sample in each respective RNA storage environment, samples were stored in a thermal cycler at 60° C. for about 48 hours and then analyzed by denaturing agarose gel electrophoresis as shown in FIGS. 10 C and D.

### Example 25

### Accelerated RNA Stability Testing at 60° C.

In vitro transcribed RNA was diluted at a ratio of about 1:10 (about 100-300 μg/mL) in different compositions containing either 50 mM sodium acetate (pH 5.2), or a mixture of acetylcholine chloride (Sigma-Aldrich, St. Louis, MO; Product #A2661) and 50 mM sodium acetate (pH 5.2) as follows:
1. 50 mM Na-Acetate (pH 5.2)
2. 1M Acetylcholine-Cl+50 mM Na-Acetate (pH 5.2)
3. 900 mM Acetylcholine-Cl+50 mM Na-Acetate (pH 5.2)
4. 800 mM Acetylcholine-Cl+50 mM Na-Acetate (pH 5.2)
5. 700 mM Acetylcholine-Cl+50 mM Na-Acetate (pH 5.2)
6. −80° C.

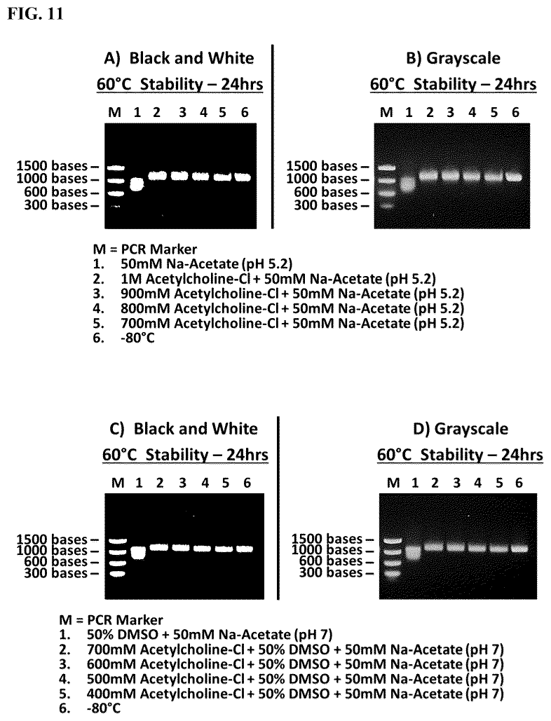

Following dilution of each sample in each respective RNA storage environment, samples were stored in a thermal cycler at 60° C. for about 24 hours and then analyzed by denaturing agarose gel electrophoresis as shown in FIGS. 11A and B.

### Example 26

### Accelerated RNA Stability Testing at 60° C.

In vitro transcribed RNA was diluted at a ratio of about 1:10 (about 100-300 μg/mL) in different compositions containing either 50% DMSO with 50 mM sodium acetate (pH 7), or a mixture of acetylcholine chloride and 50% DMSO with 50 mM sodium acetate (pH 7) as follows:
1. 50% DMSO+50 mM Na-Acetate (pH 7)
2. 700 mM Acetylcholine-Cl+50% DMSO+50 mM Na-Acetate (pH 7)
3. 600 mM Acetylcholine-Cl+50% DMSO+50 mM Na-Acetate (pH 7)
4. 500 mM Acetylcholine-Cl+50% DMSO+50 mM Na-Acetate (pH 7)
5. 400 mM Acetylcholine-Cl+50% DMSO+50 mM Na-Acetate (pH 7)
6. −80° C.

Following dilution of each sample in each respective RNA storage environment, samples were stored in a thermal cycler at 60° C. for about 24 hours and then analyzed by denaturing agarose gel electrophoresis as shown in FIGS. 11 C and D.

### Example 27

### Accelerated RNA Stability Testing at 60° C.

In vitro transcribed RNA was diluted at a ratio of about 1:10 (about 100-300 μg/mL) in different compositions containing either 5 mM sodium phosphate (pH 8) and 10 mM sodium acetate (pH 7), or a mixture of TMG (Sigma-Aldrich, St. Louis, MO; Product #B0300), NDSB-195 (Sigma-Aldrich, St. Louis, MO; Product #D0195), NDSB-221 (Hampton Research, Aliso Viejo, CA; Product #HR2-791), or L-carnitine (Cayman Chemical, Ann Arbor, MI; Product #21489), and 5 mM sodium phosphate (pH 8) and 10 mM sodium acetate (pH 7) as follows:
1. 5 mM Na-Phosphate (pH 8)+10 mM Na-Acetate (pH 7)
2. 3M TMG+5 mM Na-Phosphate (pH 8)+10 mM Na-Acetate (pH 7)
3. 1.5M NDSB-195+5 mM Na-Phosphate (pH 8)+10 mM Na-Acetate (pH 7)
4. 2M NDSB-221+5 mM Na-Phosphate (pH 8)+10 mM Na-Acetate (pH 7)
5. 2M Carnitine+5 mM Na-Phosphate (pH 8)+10 mM Na-Acetate (pH 7)
6. −80° C.

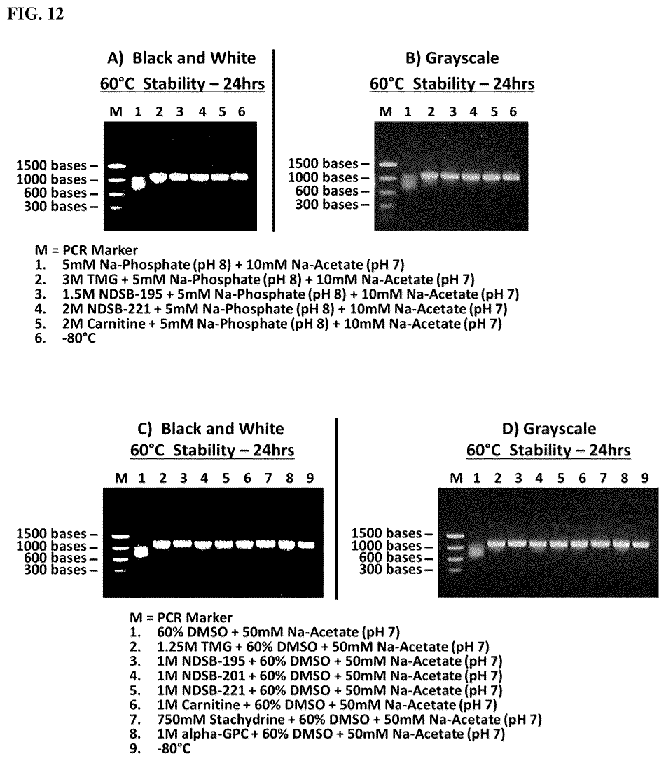

Following dilution of each sample in each respective RNA storage environment, samples were stored in a thermal cycler at 60° C. for about 24 hours and then analyzed by denaturing agarose gel electrophoresis as shown in FIGS. 12A and B.

### Example 28

### Accelerated RNA Stability Testing at 60° C.

In vitro transcribed RNA was diluted at a ratio of about 1:10 (about 100-300 μg/mL) in different compositions containing either 60% DMSO with 50 mM sodium acetate (pH 7), or a mixture of TMG, NDSB-195, NDSB-201 (Sigma-Aldrich, St. Louis, MO; Product #82804), NDSB-221, L-carnitine, stachydrine (Cayman Chemical, Ann Arbor, MI; Product #20506), or L-alpha-glycerylphosphorylcholine (alpha-GPC) (Botany Bio, San Luis Obispo, CA; Product #alphagpc-99-powder) and 60% DMSO with 50 mM sodium acetate (pH 7) as follows:
1. 60% DMSO+50 mM Na-Acetate (pH 7)
2. 1.25M TMG+60% DMSO+50 mM Na-Acetate (pH 7)
3. 1M NDSB-195+60% DMSO+50 mM Na-Acetate (pH 7)
4. 1M NDSB-201+60% DMSO+50 mM Na-Acetate (pH 7)
5. 1M NDSB-221+60% DMSO+50 mM Na-Acetate (pH 7)
6. 1M Carnitine+60% DMSO+50 mM Na-Acetate (pH 7)
7. 750 mM Stachydrine+60% DMSO+50 mM Na-Acetate (pH 7)
8. 1M alpha-GPC+60% DMSO+50 mM Na-Acetate (pH 7)
9. −80° C.

The stachydrine stock was adjusted to a pH of about 7 using NaOH. Following dilution of each sample in each respective RNA storage environment, samples were stored in a thermal cycler at 60° C. for about 24 hours and then analyzed by denaturing agarose gel electrophoresis as shown in FIGS. 12 C and D.

### Example 29

### Accelerated RNA Stability Testing at 70° C.

In vitro transcribed RNA was diluted at a ratio of about 1:10 (about 100-300 μg/mL) in different compositions containing either 50 mM Tris-HCl (pH 7), or a mixture of ˜8.5 kDa poly(2-(trimethylamino)ethyl methacrylate) chloride (PTMAEMA) (Sigma-Aldrich, St. Louis, MO; Product #657670) and 50 mM Tris-HCl (pH 7) as follows:
1. 50 mM Tris-HCl (pH 7)
2. 50 UM PTMAEMA+50 mM Tris-HCl (pH 7)
3. 40 UM PTMAEMA+50 mM Tris-HCl (pH 7)
4. 30 UM PTMAEMA+50 mM Tris-HCl (pH 7)
5. 20 UM PTMAEMA+50 mM Tris-HCl (pH 7)
6. −80° C.

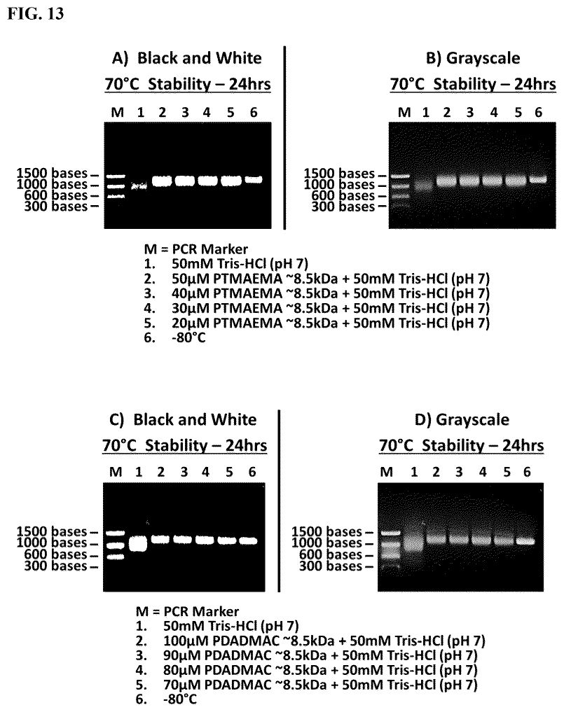

Following dilution of each sample in each respective RNA storage environment, samples were stored in a thermal cycler at 70° C. for about 24 hours and then analyzed by denaturing agarose gel electrophoresis as shown in FIGS. 13A and B.

Prior to performing gel electrophoresis, the PTMAEMA samples were incubated with 1 mM ˜8 kDa PAA sodium salt for 30 minutes at 70° C. and then incubated overnight at room temperature (about 20-30° C.) to help improve analysis by gel electrophoresis.

### Example 30

### Accelerated RNA Stability Testing at 70° C.

In vitro transcribed RNA was diluted at a ratio of about 1:10 (about 100-300 μg/mL) in different compositions containing either 50 mM Tris-HCl (pH 7), or a mixture of ˜8.5 kDa poly(diallyldimethylammonium chloride) (PDADMAC) (Polysciences Inc., Warrington, PA; Product #24828-100) and 50 mM Tris-HCl (pH 7) as follows:
1. 50 mM Tris-HCl (pH 7)
2. 100 M PDADMAC+50 mM Tris-HCl (pH 7)
3. 90 μM PDADMAC+50 mM Tris-HCl (pH 7)
4. 80 μM PDADMAC+50 mM Tris-HCl (pH 7)
5. 70 μM PDADMAC+50 mM Tris-HCl (pH 7)
6. −80° C.

Following dilution of each sample in each respective RNA storage environment, samples were stored in a thermal cycler at 70° C. for about 24 hours and then analyzed by denaturing agarose gel electrophoresis as shown in FIGS. 13 C and D.

Prior to performing gel electrophoresis, the PDADMAC samples were incubated with 1 mM ˜8 kDa PAA sodium salt overnight at room temperature (about 20-30° C.) to help improve analysis by gel electrophoresis.

### Example 31

### Accelerated RNA Stability Testing at 60° C.

In vitro transcribed RNA was diluted at a ratio of about 1:10 (about 100-300 μg/mL) in different compositions containing either 50 mM sodium acetate (pH 5.2), or a mixture of ˜10 kDa poly(2-(diethylamino)ethyl methacrylate) (PDEAEMA) (Sigma-Aldrich, St. Louis, MO; Product #910104) and 50 mM sodium acetate (pH 5.2) as follows:
1. 50 mM Na-Acetate (pH 5.2)
2. 35 μM PDEAEMA+50 mM Na-Acetate (pH 5.2)
3. 30 μM PDEAEMA+50 mM Na-Acetate (pH 5.2)
4. 25 μM PDEAEMA+50 mM Na-Acetate (pH 5.2)
5. 20 μM PDEAEMA+50 mM Na-Acetate (pH 5.2)
6. −80° C.

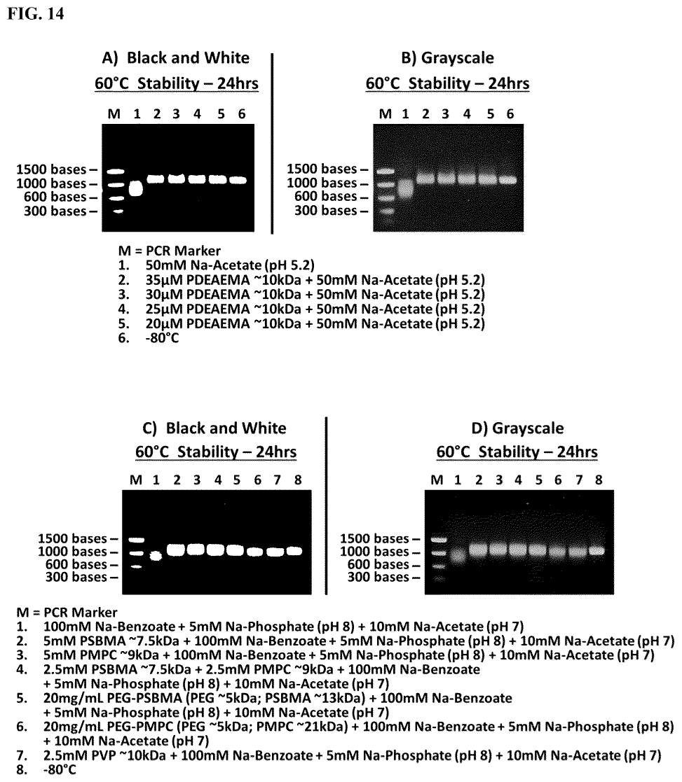

The PDEAEMA stock solution was made in 50 mM sodium acetate (pH 5.2). Following dilution of each sample in each respective RNA storage environment, samples were stored in a thermal cycler at 60° C. for about 24 hours and then analyzed by denaturing agarose gel electrophoresis as shown in FIGS. 14A and B.

Prior to performing gel electrophoresis, the PDEAEMA samples were incubated with 2.5 mM ˜8 kDa PAA sodium salt for about 1 hour at room temperature (about 20-30° C.) to help improve analysis by gel electrophoresis.

### Example 32

### Accelerated RNA Stability Testing at 60° C.

In vitro transcribed RNA was diluted at a ratio of about 1:10 (about 100-300 μg/mL) in different compositions containing either 100 mM sodium benzoate with 5 mM sodium phosphate (pH 8) and 10 mM sodium acetate (pH 7), or ˜7.5 kDa poly(2-(N-3-sulfopropyl-N,N-dimethyl ammonium)ethyl methacrylate) (PSBMA) (Sigma-Aldrich, St. Louis, MO; Product #922390), ˜9 kDa poly(2-methacryloyloxyethyl phosphorylcholine) (PMPC) (Sigma-Aldrich, St. Louis, MO; Product #922749), a combination of both ˜7.5 kDa PSBMA and ˜9 kDa PMPC, PEG-block-PSBMA block copolymer (PEG-PSBMA) (PEG Mn 5,000; PSBMA Mn 13,000) (Sigma-Aldrich, St. Louis, MO; Product #925640), PEG-block-PMPC block copolymer (PEG-PMPC) (PEG Mn 5,000; PMPC Mn 21,000) (Sigma-Aldrich, St. Louis, MO; Product #925632), or ˜10 kDa polyvinylpyrrolidone (PVP) (Sigma-Aldrich, St. Louis, MO; Product #P2307) and 100 mM sodium benzoate with 5 mM sodium phosphate (pH 8) and 10 mM sodium acetate (pH 7) as follows:
1. 100 mM Na-Benzoate+5 mM Na-Phosphate (pH 8)+10 mM Na-Acetate (pH 7)
2. 5 mM PSBMA+100 mM Na-Benzoate+5 mM Na-Phosphate (pH 8)+10 mM Na-Acetate (pH 7)
3. 5 mM PMPC+100 mM Na-Benzoate+5 mM Na-Phosphate (pH 8)+10 mM Na-Acetate (pH 7)
4. 2.5 mM PSBMA+2.5 mM PMPC+100 mM Na-Benzoate+5 mM Na-Phosphate (pH 8)+10 mM Na-Acetate (pH 7)
5. 20 mg/mL PEG-PSBMA+100 mM Na-Benzoate+5 mM Na-Phosphate (pH 8)+10 mM Na-Acetate (pH 7)
6. 20 mg/mL PEG-PMPC+100 mM Na-Benzoate+5 mM Na-Phosphate (pH 8)+10 mM Na-Acetate (pH 7)
7. 2.5 mM PVP+100 mM Na-Benzoate+5 mM Na-Phosphate (pH 8)+10 mM Na-Acetate (pH 7)
8. −80° C.

Following dilution of each sample in each respective RNA storage environment, samples were stored in a thermal cycler at 60° C. for about 24 hours and then analyzed by denaturing agarose gel electrophoresis as shown in FIGS. 14 C and D.

Prior to performing gel electrophoresis, the samples were incubated with 1 mM ˜8 kDa PAA sodium salt for about 1 hour at room temperature (about 20-30° C.) to help improve analysis by gel electrophoresis.

### Example 33

### Accelerated RNA Stability Testing at 60° C.

In vitro transcribed RNA was diluted at a ratio of about 1:10 (about 100-300 μg/mL) in different compositions containing either 50 mM sodium acetate (pH 5.2), or a mixture of ˜8 kDa poly(acrylic acid, sodium salt) (PAA) (Sigma-Aldrich, St. Louis, MO; Product #416029) and 50 mM sodium acetate (pH 5.2) as follows:
1. 50 mM Na-Acetate (pH 5.2)
2. 5 mM PAA sodium salt+50 mM Na-Acetate (pH 5.2)
3. 2.5 mM PAA sodium salt+50 mM Na-Acetate (pH 5.2)
4. 1 mM PAA sodium salt+50 mM Na-Acetate (pH 5.2)
5. 500 μM PAA sodium salt+50 mM Na-Acetate (pH 5.2)
6. −80° C.

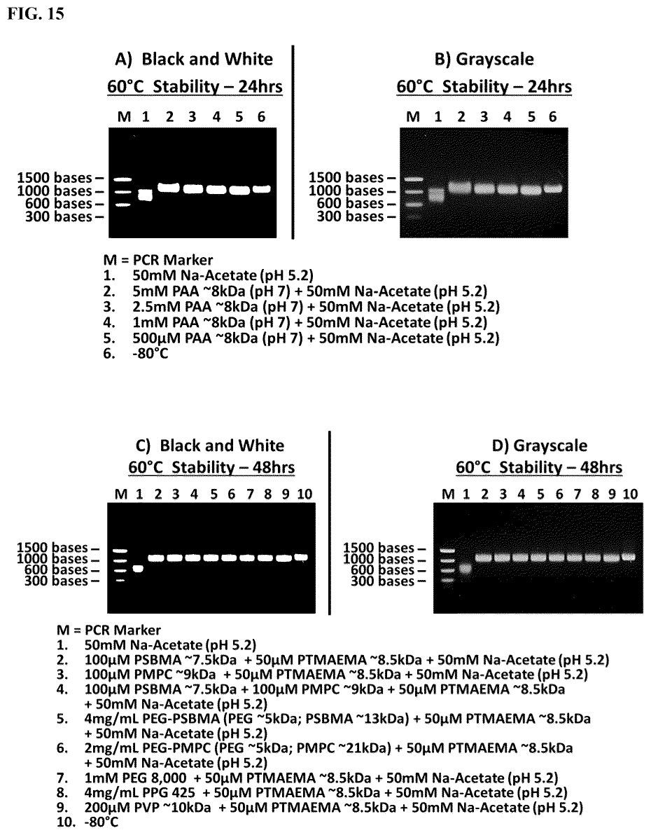

Following dilution of each sample in each respective RNA storage environment, samples were stored in a thermal cycler at 60° C. for about 24 hours and then analyzed by denaturing agarose gel electrophoresis as shown in FIGS. 15A and B.

### Example 34

### Accelerated RNA Stability Testing at 60° C.

In vitro transcribed RNA was diluted at a ratio of about 1:10 (about 100-300 μg/mL) in different compositions containing either 50 mM sodium acetate (pH 5.2), or 50 μM ˜8.5 kDa PTMAEMA in combination with ˜7.5 kDa PSBMA, ˜9 kDa PMPC, a combination of both ˜7.5 kDa PSBMA and ˜9 kDa PMPC, PEG-PSBMA (PEG Mn 5,000; PSBMA Mn 13,000), PEG-PMPC (PEG Mn 5,000; PMPC Mn 21,000), poly(ethylene glycol) 8,000 (PEG) (Sigma-Aldrich, St. Louis, MO; Product #89510), poly(propylene glycol) 425 (PPG) (Mn ˜425) (Sigma-Aldrich, St. Louis, MO; Product #202304), or ˜10 kDa PVP, and 50 mM sodium acetate (pH 5.2) as follows:
1. 50 mM Na-Acetate (pH 5.2)
2. 100 μM PSBMA+50 μM PTMAEMA+50 mM Na-Acetate (pH 5.2)
3. 100 μM PMPC+50 μM PTMAEMA+50 mM Na-Acetate (pH 5.2)
4. 100 μM PSBMA+100 μM PMPC+50 UM PTMAEMA+50 mM Na-Acetate (pH 5.2)
5. 4 mg/mL PEG-PSBMA+50 μM PTMAEMA+50 mM Na-Acetate (pH 5.2)
6. 2 mg/mL PEG-PMPC+50 μM PTMAEMA+50 mM Na-Acetate (pH 5.2)
7. 1 mM PEG 8,000+50 UM PTMAEMA+50 mM Na-Acetate (pH 5.2)
8. 4 mg/mL PPG 425+50 μM PTMAEMA+50 mM Na-Acetate (pH 5.2)
9. 200 UM PVP+50 UM PTMAEMA+50 mM Na-Acetate (pH 5.2)
10. −80° C.

Following dilution of each sample in each respective RNA storage environment, samples were stored in a thermal cycler at 60° C. for about 48 hours and then analyzed by denaturing agarose gel electrophoresis as shown in FIGS. 15 C and D.

Prior to performing gel electrophoresis, the samples were incubated with 2.5 mM ˜8 kDa PAA sodium salt for 30 minutes at 70° C. to help improve analysis by gel electrophoresis.

### Example 35

### Accelerated RNA Stability Testing at 60° C.

In vitro transcribed RNA was diluted at a ratio of about 1:10 (about 100-300 μg/mL) in different compositions containing either 50 mM sodium acetate (pH 5.2), or 100 μM ˜8.5 kDa PDADMAC in combination with ˜7.5 kDa PSBMA, ˜9 kDa PMPC, a combination of both ˜7.5 kDa PSBMA and ˜9 kDa PMPC, PEG-PSBMA (PEG Mn 5,000; PSBMA Mn 13,000), PEG-PMPC (PEG Mn 5,000; PMPC Mn 21,000), PEG 8,000, PPG 425, or ˜10 kDa PVP, and 50 mM sodium acetate (pH 5.2) as follows:
1. 50 mM Na-Acetate (pH 5.2)
2. 100 μM PSBMA+100 μM PDADMAC+50 mM Na-Acetate (pH 5.2)
3. 100 μM PMPC+100 μM PDADMAC+50 mM Na-Acetate (pH 5.2)
4. 100 μM PSBMA+100 μM PMPC+100 μM PDADMAC+50 mM Na-Acetate (pH 5.2)
5. 4 mg/mL PEG-PSBMA+100 μM PDADMAC+50 mM Na-Acetate (pH 5.2)
6. 2 mg/mL PEG-PMPC+100 μM PDADMAC+50 mM Na-Acetate (pH 5.2)
7. 1 mM PEG 8,000+100 μM PDADMAC+50 mM Na-Acetate (pH 5.2)
8. 4 mg/mL PPG 425+100 μM PDADMAC+50 mM Na-Acetate (pH 5.2)
9. 200 μM PVP+100 μM PDADMAC+50 mM Na-Acetate (pH 5.2)
10. −80° C.

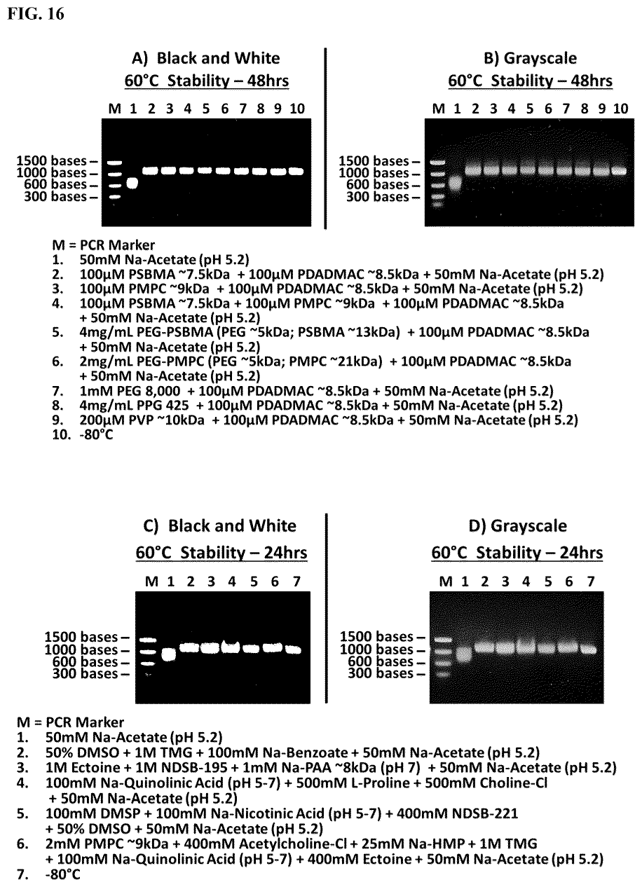

Following dilution of each sample in each respective RNA storage environment, samples were stored in a thermal cycler at 60° C. for about 48 hours and then analyzed by denaturing agarose gel electrophoresis as shown in FIGS. 16A and B.

Prior to performing gel electrophoresis, the samples were incubated with 2.5 mM ˜8 kDa PAA sodium salt for about 1 hour at room temperature (about 20-30° C.) to help improve analysis by gel electrophoresis.

### Example 36

### Accelerated RNA Stability Testing at 60° C.

In vitro transcribed RNA was diluted at a ratio of about 1:10 (about 100-300 μg/mL) in different compositions containing either 50 mM sodium acetate (pH 5.2), or one or more compounds in compositions with 50 mM sodium acetate (pH 5.2) as follows:
1. 50 mM Na-Acetate (pH 5.2)
2. 50% DMSO+1M TMG+100 mM Na-Benzoate+50 mM Na-Acetate (pH 5.2)
3. 1M Ectoine+1M NDSB-195+1 mM PAA ˜8 kDa sodium salt+50 mM Na-Acetate (pH 5.2)
4. 100 mM Na-Quinolinic Acid (pH 7)+500 mM L-Proline+500 mM Choline-Cl+50 mM Na-Acetate (pH 5.2)
5. 100 mM DMSP+100 mM Na-Nicotinic Acid (pH 7)+400 mM NDSB-221+50% DMSO+50 mM Na-Acetate (pH 5.2)
6. 2 mM PMPC ˜9 kDa+400 mM Acetylcholine-Cl+25 mM Na-HMP+1M TMG+100 mM Na-Quinolinic Acid (pH 7)+400 mM Ectoine+50 mM Na-Acetate (pH 5.2)
7. −80° C.

The quinolinic acid, nicotinic acid, and DMSP stocks were adjusted to a pH of about 5-7 using NaOH. Following dilution of each sample in each respective RNA storage environment, samples were stored in a thermal cycler at 60° C. for about 24 hours and then analyzed by denaturing agarose gel electrophoresis as shown in FIGS. 16 C and D.

Prior to performing gel electrophoresis, sample 5 comprising DMSP was incubated with 1 mM ˜8 kDa PAA sodium salt for about 1 hour at room temperature (about 20-30° C.) to help improve analysis by gel electrophoresis.

### Example 37

### Accelerated RNA Stability Testing at 60° C.

In vitro transcribed RNA was diluted at a ratio of about 1:10 (about 100-300 μg/mL) in different compositions containing either 50 mM sodium acetate (pH 5.2), or one or more compounds in compositions with 50 mM sodium acetate (pH 5.2) as follows:
1. 50 mM Na-Acetate (pH 5.2)
2. 50% DMSO+1M TMG+100 mM Na-Benzoate+50 mM Na-Acetate (pH 5.2)
3. 100 mM Na-TMP+500 mM Choline-Cl+1 mM PAA ˜8 kDa sodium salt+50 mM Na-Acetate (pH 5.2)
4. 100 mM DMSP+100 mM Na-Quinolinic Acid (pH 7)+600 mM NDSB-195+50% DMSO+50 mM Na-Acetate (pH 5.2)
5. 1 mM PSBMA ˜7.5 kDa+100 mM Acetylcholine-Cl+40 mM Na-HMP+1M TMG+100 mM Na-Benzoate+400 mM Ectoine+50 mM Na-Acetate (pH 5.2)
6. −80° C.

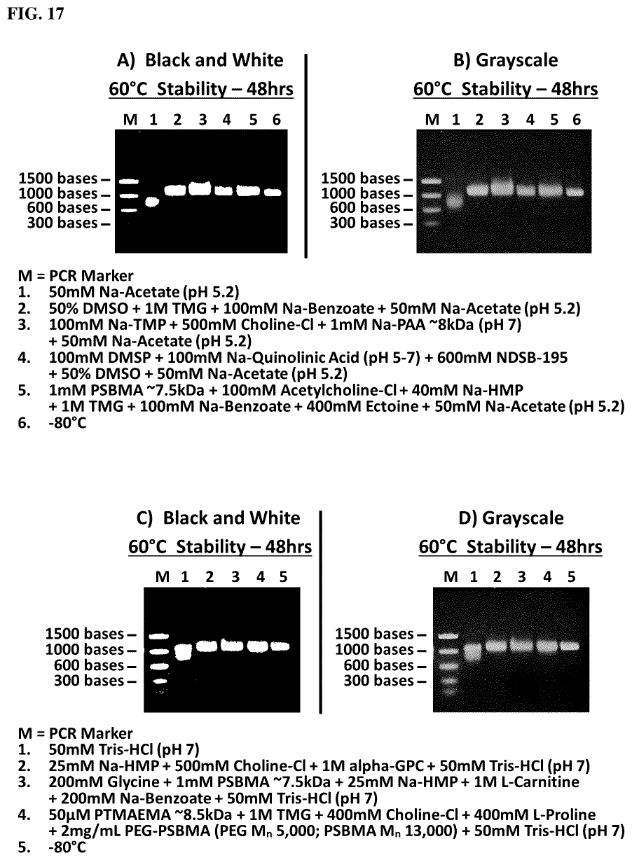

The quinolinic acid and DMSP stocks were adjusted to a pH of about 5-7 using NaOH. Following dilution of each sample in each respective RNA storage environment, samples were stored in a thermal cycler at 60° C. for about 48 hours and then analyzed by denaturing agarose gel electrophoresis as shown in FIGS. 17A and B.

Prior to performing gel electrophoresis, sample 4 comprising DMSP was incubated with 1 mM ˜8 kDa PAA sodium salt for about 1 hour at room temperature (about 20-30° C.) to help improve analysis by gel electrophoresis.

### Example 38

### Accelerated RNA Stability Testing at 60° C.

In vitro transcribed RNA was diluted at a ratio of about 1:10 (about 100-300 μg/mL) in different compositions containing either 50 mM Tris-HCl (pH 7), or one or more compounds in compositions with 50 mM Tris-HCl (pH 7) as follows:
1. 50 mM Tris-HCl (pH 7)
2. 25 mM Na-HMP+500 mM Choline-Cl+1M alpha-GPC+50 mM Tris-HCl (pH 7)
3. 200 mM Glycine+1 mM PSBMA ˜7.5 kDa+25 mM Na-HMP+1M L-Carnitine+200 mM Na-Benzoate+50 mM Tris-HCl (pH 7)
4. 50 μM PTMAEMA ˜8.5 kDa+1M TMG+400 mM Choline-Cl+400 mM L-Proline+2 mg/mL PEG-PSBMA (PEG Mn 5,000; PSBMA Mn 13,000)+50 mM Tris-HCl (pH 7)
5. −80° C.

Following dilution of each sample in each respective RNA storage environment, samples were stored in a thermal cycler at 60° C. for about 48 hours and then analyzed by denaturing agarose gel electrophoresis as shown in FIGS. 17 C and D.

Prior to performing gel electrophoresis, sample 4 comprising PTMAEMA was incubated with 2.5 mM ˜8 kDa PAA sodium salt for 30 minutes at 70° C. to help improve analysis by gel electrophoresis.

### Example 39

### Accelerated RNA Stability Testing at 60° C.

In vitro transcribed RNA was diluted at a ratio of about 1:10 (about 100-300 μg/mL) in different compositions containing either 50 mM Tris-HCl (pH 7), or one or more compounds in compositions with 50 mM Tris-HCl (pH 7) as follows:
1. 50 mM Tris-HCl (pH 7)
2. 40 mM Na-HMP+500 mM Choline-Cl+1M alpha-GPC+50 mM Tris-HCl (pH 7)
3. 500 mM Choline-Cl+50% DMSO+1 mM PMPC ˜9 kDa+500 mM NDSB-195+50 mM Tris-HCl (pH 7)
4. 50 UM PTMAEMA ˜8.5 kDa+100 mM TMG+100 mM Ectoine+400 mM alpha-GPC+100 μM PSBMA ˜7.5 kDa+50 mM Tris-HCl (pH 7)
5. −80° C.

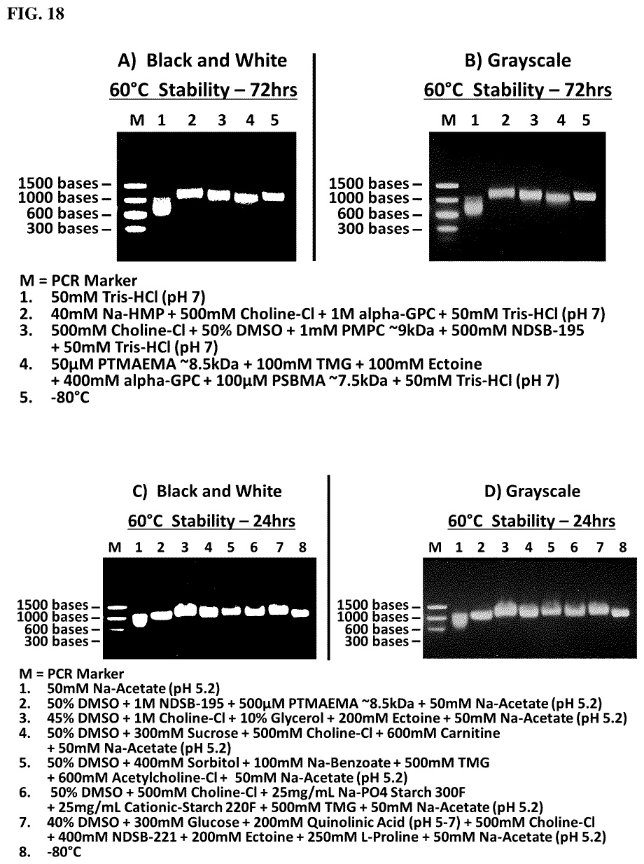

Following dilution of each sample in each respective RNA storage environment, samples were stored in a thermal cycler at 60° C. for about 72 hours and then analyzed by denaturing agarose gel electrophoresis as shown in FIGS. 18A and B.

Prior to performing gel electrophoresis, sample 4 comprising PTMAEMA was incubated with 2.5 mM ˜8 kDa PAA sodium salt for 30 minutes at 70° C. to help improve analysis by gel electrophoresis.

### Example 40

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 18 C and D.

M=PCR Marker
1. 50 mM Na-Acetate (pH 5.2)
2. 50% DMSO+1M NDSB-195+500 UM PTMAEMA ˜8.5 kDa+50 mM Na-Acetate (pH 5.2)
3. 45% DMSO+1M Choline-Cl+10% Glycerol+200 mM Ectoine+50 mM Na-Acetate (pH 5.2)
4. 50% DMSO+300 mM Sucrose+500 mM Choline-Cl+600 mM L-Carnitine+50 mM Na-Acetate (pH 5.2)
5. 50% DMSO+400 mM Sorbitol+100 mM Na-Benzoate+500 mM TMG+600 mM Acetylcholine-Cl+50 mM Na-Acetate (pH 5.2)
6. 50% DMSO+500 mM Choline-Cl+25 mg/mL Na—PO4 Starch 300F+25 mg/mL Cationic-Starch 220F+500 mM TMG+50 mM Na-Acetate (pH 5.2)
7. 40% DMSO+300 mM Glucose+200 mM Quinolinic Acid (pH 5-7)+500 mM Choline-Cl+400 mM NDSB-221+200 mM Ectoine+250 mM L-Proline+50 mM Na-Acetate (pH 5.2)
8. −80° C.

Following dilution of each sample in each respective RNA storage environment, samples were stored in a thermal cycler at 60° C. for about 24 hours and then analyzed by denaturing agarose gel electrophoresis as shown in FIGS. 18 C and D.

Prior to performing gel electrophoresis, sample 2 comprising PTMAEMA was incubated with 2.5 mM ˜8 kDa PAA sodium salt for 30 minutes at 70° C. to help improve analysis by gel electrophoresis.

The following substances in one or more compositions were purchased as follows: Sucrose (Sigma-Aldrich, St. Louis, MO; Product #84097); D-Glucose (Sigma-Aldrich, Product #G5767) Trehalose (Cayman Chemical, Ann Arbor, MI, Product #20517), D-Sorbitol (Sigma-Aldrich, Product #S1876) and Glycerol (Sigma-Aldrich, Product #G5516).

### Example 41

### Accelerated RNA Stability Testing at 60° C.

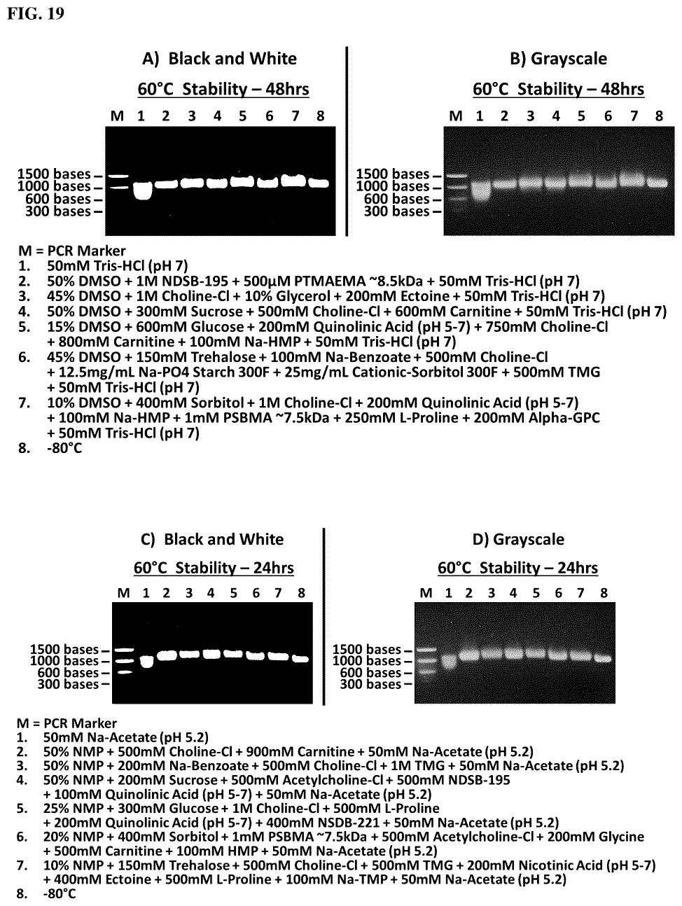

In vitro transcribed RNA was diluted at a ratio of about 1:10 (about 100-300 μg/mL) in different compositions containing either 50 mM Tris-HCl (pH 7), or one or more compounds in compositions with 50 mM Tris-HCl (pH 7) as shown in FIGS. 19A and B:
M=PCR Marker
1. 50 mM Tris-HCl (pH 7)
2. 50% DMSO+1M NDSB-195+500 μM PTMAEMA ˜8.5 kDa+50 mM Tris-HCl (pH 7)
3. 45% DMSO+1M Choline-Cl+10% Glycerol+200 mM Ectoine+50 mM Tris-HCl (pH 7)
4. 50% DMSO+300 mM Sucrose+500 mM Choline-Cl+600 mM L-Carnitine+50 mM Tris-HCl (pH 7)
5. 15% DMSO+600 mM Glucose+200 mM Quinolinic Acid (pH 5-7)+750 mM Choline-Cl+800 mM Carnitine+100 mM Na-HMP+50 mM Tris-HCl (pH 7)
6. 45% DMSO+150 mM Trehalose+100 mM Na-Benzoate+500 mM Choline-Cl+12.5 mg/mL Na—PO4 Starch 300F+25 mg/mL Cationic-Sorbitol 300F+500 mM TMG+50 mM Tris-HCl (pH 7)
7. 10% DMSO+400 mM Sorbitol+1M Choline-Cl+200 mM Quinolinic Acid (pH 5-7)+100 mM Na-HMP+1 mM PSBMA ˜7.5 kDa+250 mM L-Proline+200 mM Alpha-GPC+50 mM Tris-HCl (pH 7)
8. −80° C.

Following dilution of each sample in each respective RNA storage environment, samples were stored in a thermal cycler at 60° C. for about 48 hours and then analyzed by denaturing agarose gel electrophoresis as shown in FIGS. 19A and B.

Prior to performing gel electrophoresis, sample 2 comprising PTMAEMA was incubated with 2.5 mM ˜8 kDa PAA sodium salt for 30 minutes at 70° C. to help improve analysis by gel electrophoresis.

### Example 42

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 19 C and D:
M=PCR Marker
1. 50 mM Na-Acetate (pH 5.2)
2. 50% NMP+500 mM Choline-Cl+900 mM L-Carnitine+50 mM Na-Acetate (pH 5.2)
3. 50% NMP+200 mM Na-Benzoate+500 mM Choline-Cl+1M TMG+50 mM Na-Acetate (pH 5.2)
4. 50% NMP+200 mM Sucrose+500 mM Acetylcholine-Cl+500 mM NDSB-195+100 mM Quinolinic Acid (pH 5-7)+50 mM Na-Acetate (pH 5.2)
5. 25% NMP+300 mM Glucose+1M Choline-Cl+500 mM L-Proline+200 mM Quinolinic Acid (pH 5-7)+400 mM NSDB-221+50 mM Na-Acetate (pH 5.2)
6. 20% NMP+400 mM Sorbitol+1 mM PSBMA ˜7.5 kDa+500 mM Acetylcholine-Cl+200 mM Glycine+500 mM L-Carnitine+100 mM HMP+50 mM Na-Acetate (pH 5.2)
7. 10% NMP+150 mM Trehalose+500 mM Choline-Cl+500 mM TMG+200 mM Nicotinic Acid (pH 5-7)+400 mM Ectoine+500 mM L-Proline+100 mM Na-TMP+50 mM Na-Acetate (pH 5.2)
8. −80° C.

Following dilution of each sample in each respective RNA storage environment, samples were stored in a thermal cycler at 60° C. for about 24 hours and then analyzed by denaturing agarose gel electrophoresis as shown in FIGS. 19 C and D.

### Example 43

### Accelerated RNA Stability Testing at 60° C.

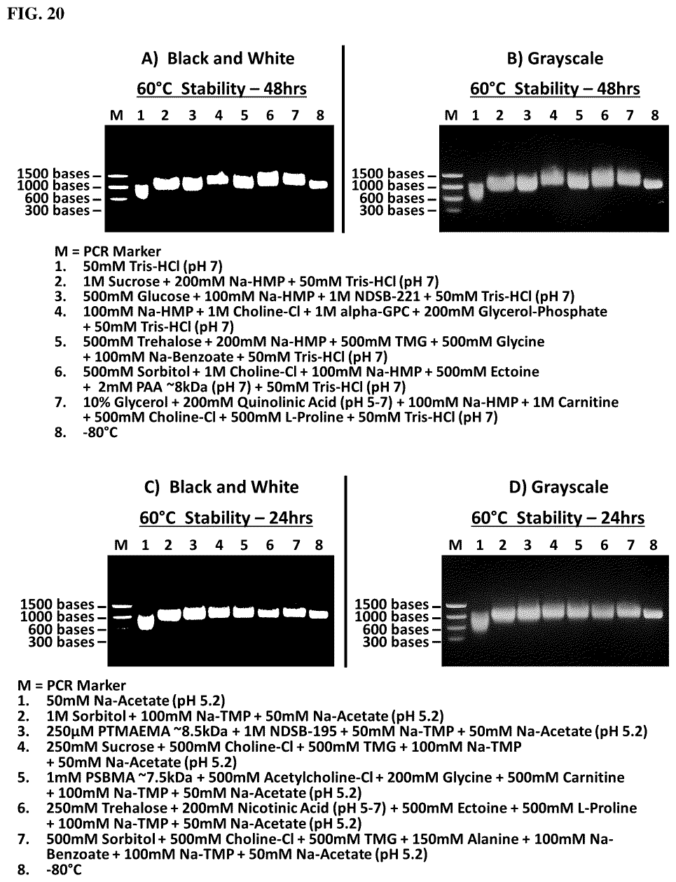

In vitro transcribed RNA was diluted at a ratio of about 1:10 (about 100-300 μg/mL) in different compositions containing either 50 mM Tris-HCl (pH 7), or one or more compounds in compositions with 50 mM Tris-HCl (pH 7) as shown in FIGS. 20A and B:
M=PCR Marker
1. 50 mM Tris-HCl (pH 7)
2. 1M Sucrose+200 mM Na-HMP+50 mM Tris-HCl (pH 7)
3. 500 mM Glucose+100 mM Na-HMP+1M NDSB-221+50 mM Tris-HCl (pH 7)
4. 100 mM Na-HMP+1M Choline-Cl+1M alpha-GPC+200 mM Glycerol-Phosphate+50 mM Tris-HCl (pH 7)
5. 500 mM Trehalose+200 mM Na-HMP+500 mM TMG+500 mM Glycine+100 mM Na-Benzoate+50 mM Tris-HCl (pH 7)
6. 500 mM Sorbitol+1M Choline-Cl+100 mM Na-HMP+500 mM Ectoine+2 mM PAA ˜8 kDa (pH 7)+50 mM Tris-HCl (pH 7)
7. 10% Glycerol+200 mM Quinolinic Acid (pH 5-7)+100 mM Na-HMP+1M L-Carnitine+500 mM Choline-Cl+500 mM L-Proline+50 mM Tris-HCl (pH 7)
8. −80° C.

Following dilution of each sample in each respective RNA storage environment, samples were stored in a thermal cycler at 60° C. for about 48 hours and then analyzed by denaturing agarose gel electrophoresis as shown in FIGS. 20A and B.

### Example 44

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 20 C and D:
M=PCR Marker
1. 50 mM Na-Acetate (pH 5.2)
2. 1M Sorbitol+100 mM Na-TMP+50 mM Na-Acetate (pH 5.2)
3. 250 μM PTMAEMA ˜8.5 kDa+1M NDSB-195+50 mM Na-TMP+50 mM Na-Acetate (pH 5.2)
4. 250 mM Sucrose+500 mM Choline-Cl+500 mM TMG+100 mM Na-TMP+50 mM Na-Acetate (pH 5.2)
5. 1 mM PSBMA ˜7.5 kDa+500 mM Acetylcholine-Cl+200 mM Glycine+500 mM Carnitine+100 mM Na-TMP+50 mM Na-Acetate (pH 5.2)
6. 250 mM Trehalose+200 mM Nicotinic Acid (pH 5-7)+500 mM Ectoine+500 mM L-Proline+100 mM Na-TMP+50 mM Na-Acetate (pH 5.2)
7. 500 mM Sorbitol+500 mM Choline-Cl+500 mM TMG+150 mM Alanine+100 mM Na-Benzoate+100 mM Na-TMP+50 mM Na-Acetate (pH 5.2)
8. −80° C.

Following dilution of each sample in each respective RNA storage environment, samples were stored in a thermal cycler at 60° C. for about 24 hours and then analyzed by denaturing agarose gel electrophoresis as shown in FIGS. 20 C and D.

Prior to performing gel electrophoresis, sample 3 comprising PTMAEMA was incubated with 2.5 mM ˜8 kDa PAA sodium salt for 30 minutes at 70° C. to help improve analysis by gel electrophoresis.

### Example 45

### Accelerated RNA Stability Testing at 60° C.

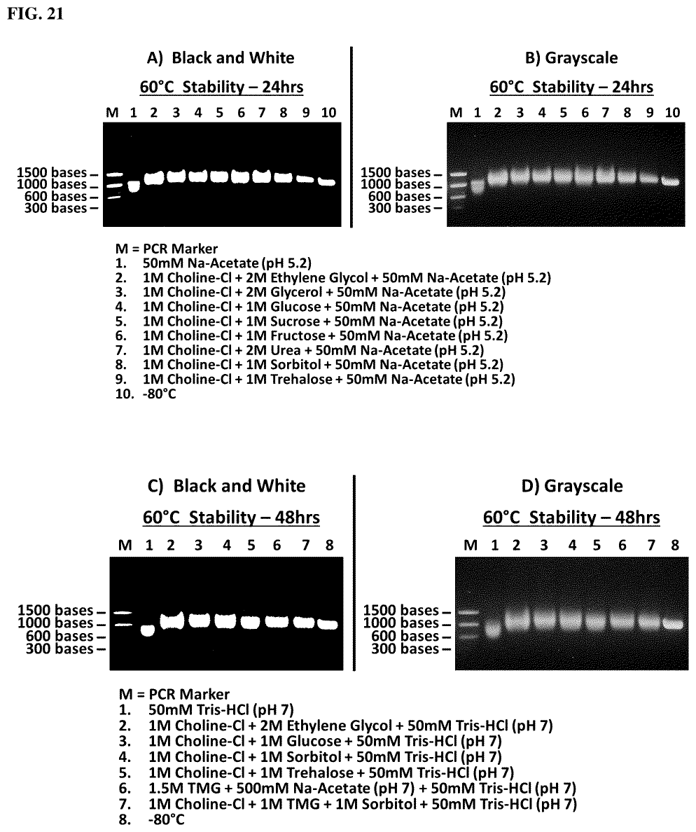

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 21A and B:
M=PCR Marker
1. 50 mM Na-Acetate (pH 5.2)
2. 1M Choline-Cl+2M Ethylene Glycol+50 mM Na-Acetate (pH 5.2)
3. 1M Choline-Cl+2M Glycerol+50 mM Na-Acetate (pH 5.2)
4. 1M Choline-Cl+1M Glucose+50 mM Na-Acetate (pH 5.2)
5. 1M Choline-Cl+1M Sucrose+50 mM Na-Acetate (pH 5.2)
6. 1M Choline-Cl+1M Fructose+50 mM Na-Acetate (pH 5.2)
7. 1M Choline-Cl+2M Urea+50 mM Na-Acetate (pH 5.2)
8. 1M Choline-Cl+1M Sorbitol+50 mM Na-Acetate (pH 5.2)
9. 1M Choline-Cl+1M Trehalose+50 mM Na-Acetate (pH 5.2)
10. −80° C.

Following dilution of each sample in each respective RNA storage environment, samples were stored in a thermal cycler at 60° C. for about 24 hours and then analyzed by denaturing agarose gel electrophoresis as shown in FIGS. 21A and B.

The following substances in one or more compositions were purchased as follows: Urea (Sigma-Aldrich, Product #U5378); Ethylene Glycol (Sigma-Aldrich, Product #102466).

### Example 46

### Accelerated RNA Stability Testing at 60° C.

In vitro transcribed RNA was diluted at a ratio of about 1:10 (about 100-300 μg/mL) in different compositions containing either 50 mM Tris-HCl (pH 7), or one or more compounds in compositions with 50 mM Tris-HCl (pH 7) as shown in FIGS. 21 C and D:
M=PCR Marker
1. 50 mM Tris-HCl (pH 7)
2. 1M Choline-Cl+2M Ethylene Glycol+50 mM Tris-HCl (pH 7)
3. 1M Choline-Cl+1M Glucose+50 mM Tris-HCl (pH 7)
4. 1M Choline-Cl+1M Sorbitol+50 mM Tris-HCl (pH 7)
5. 1M Choline-Cl+1M Trehalose+50 mM Tris-HCl (pH 7)
6. 1.5M TMG+500 mM Na-Acetate (pH 7)+50 mM Tris-HCl (pH 7)
7. 1M Choline-Cl+1M TMG+1M Sorbitol+50 mM Tris-HCl (pH 7)
8. −80° C.

Following dilution of each sample in each respective RNA storage environment, samples were stored in a thermal cycler at 60° C. for about 48 hours and then analyzed by denaturing agarose gel electrophoresis as shown in FIGS. 21 C and D.

### Example 47

### Accelerated RNA Stability Testing at 60° C.

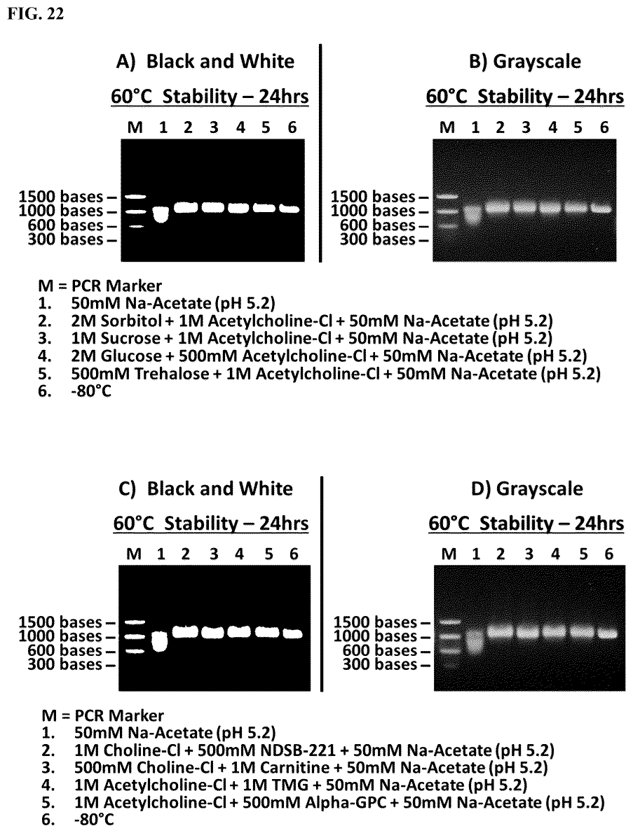

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 22A and B:
M=PCR Marker
1. 50 mM Na-Acetate (pH 5.2)
2. 2M Sorbitol+1M Acetylcholine-Cl+50 mM Na-Acetate (pH 5.2)
3. 1M Sucrose+1M Acetylcholine-Cl+50 mM Na-Acetate (pH 5.2)
4. 2M Glucose+500 mM Acetylcholine-Cl+50 mM Na-Acetate (pH 5.2)
5. 500 mM Trehalose+1M Acetylcholine-Cl+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 22A and B.

### Example 48

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 22 C and D:
M=PCR Marker
1. 50 mM Na-Acetate (pH 5.2)
2. 1M Choline-Cl+500 mM NDSB-221+50 mM Na-Acetate (pH 5.2)
3. 500 mM Choline-Cl+1M Carnitine+50 mM Na-Acetate (pH 5.2)
4. 1M Acetylcholine-Cl+1M TMG+50 mM Na-Acetate (pH 5.2)
5. 1M Acetylcholine-Cl+500 mM Alpha-GPC+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 22 C and D.

### Example 49

Accelerated RNA Stability Testing at 60° C.

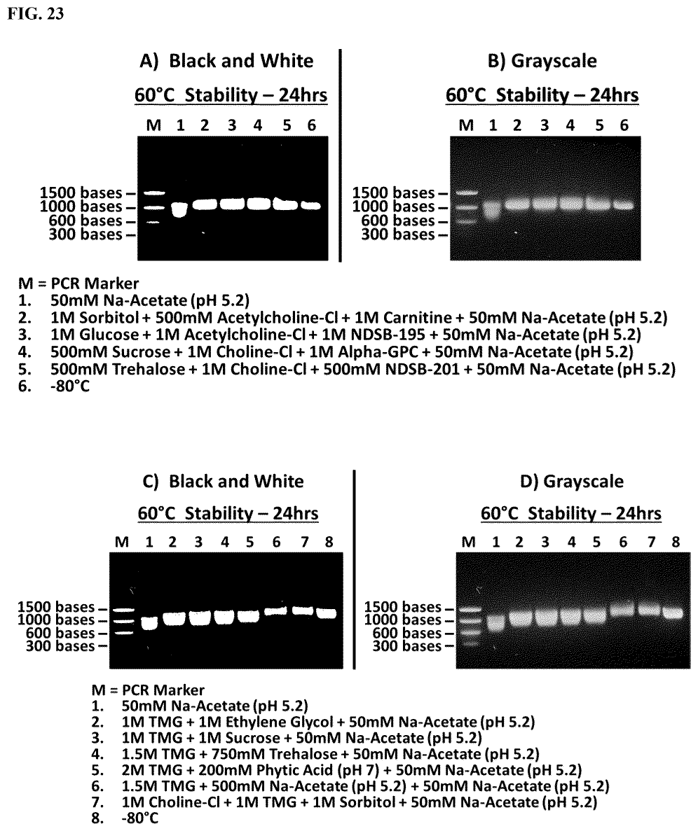

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 23A and B:
M=PCR Marker
1. 50 mM Na-Acetate (pH 5.2)
2. 1M Sorbitol+500 mM Acetylcholine-Cl+1M Carnitine+50 mM Na-Acetate (pH 5.2)
3. 1M Glucose+1M Acetylcholine-Cl+1M NDSB-195+50 mM Na-Acetate (pH 5.2)
4. 500 mM Sucrose+1M Choline-Cl+1M Alpha-GPC+50 mM Na-Acetate (pH 5.2)
5. 500 mM Trehalose+1M Choline-Cl+500 mM NDSB-201+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 23A and B.

### Example 50

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 23 C and D:
M=PCR Marker
1. 50 mM Na-Acetate (pH 5.2)
2. 1M TMG+1M Ethylene Glycol+50 mM Na-Acetate (pH 5.2)
3. 1M TMG+1M Sucrose+50 mM Na-Acetate (pH 5.2)
4. 1.5M TMG+750 mM Trehalose+50 mM Na-Acetate (pH 5.2)
5. 2M TMG+200 mM Phytic Acid (pH 7)+50 mM Na-Acetate (pH 5.2)
6. 1.5M TMG+500 mM Na-Acetate (pH 5.2)+50 mM Na-Acetate (pH 5.2)
7. 1M Choline-Cl+1M TMG+1M Sorbitol+50 mM Na-Acetate (pH 5.2)
8. −80° C.

Following dilution of each sample in each respective RNA storage environment, samples were stored in a thermal cycler at 60° C. for about 24 hours and then analyzed by denaturing agarose gel electrophoresis as shown in FIGS. 23 C and D.

The pH of phytic acid was adjusted to about 7 using NaOH.

The following substances in one or more compositions were purchased as follows: Phytic Acid (Sigma-Aldrich, Product #593648).

### Example 51

### Accelerated RNA Stability Testing at 60° C.

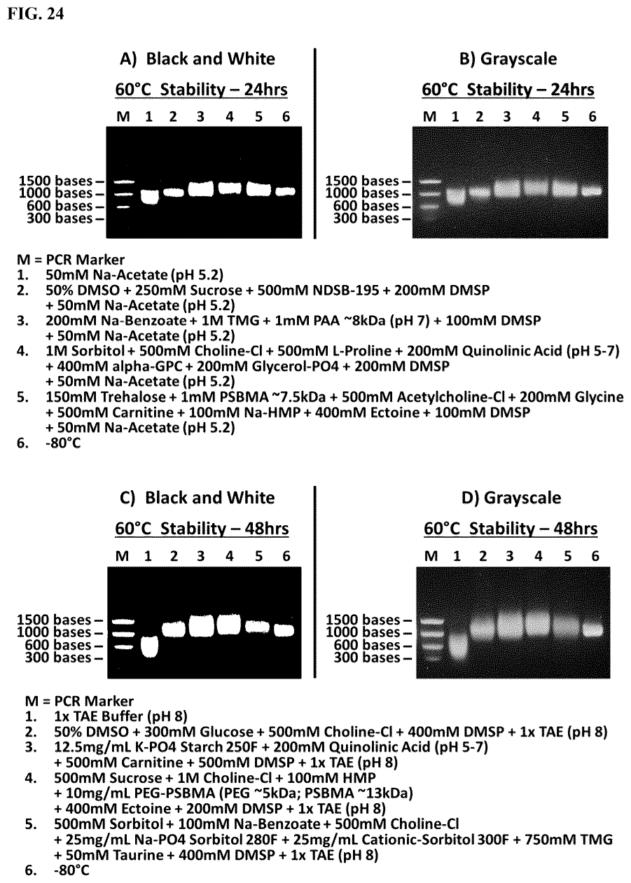

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 24A and B:
M=PCR Marker
1. 50 mM Na-Acetate (pH 5.2)
2. 50% DMSO+250 mM Sucrose+500 mM NDSB-195+200 mM DMSP+50 mM Na-Acetate (pH 5.2)
3. 200 mM Na-Benzoate+1M TMG+1 mM PAA ˜8 kDa (pH 7)+100 mM DMSP+50 mM Na-Acetate (pH 5.2)
4. 1M Sorbitol+500 mM Choline-Cl+500 mM L-Proline+200 mM Quinolinic Acid (pH 5-7)+400 mM alpha-GPC+200 mM Glycerol-PO4+200 mM DMSP+50 mM Na-Acetate (pH 5.2)
5. 150 mM Trehalose+1 mM PSBMA ˜7.5 kDa+500 mM Acetylcholine-Cl+200 mM Glycine+500 mM L-Carnitine+100 mM Na-HMP+400 mM Ectoine+100 mM DMSP+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 24A and B.
PSMBA was ˜7.5 kDa

### Example 52

### Accelerated RNA Stability Testing at 60° C.

In vitro transcribed RNA was diluted at a ratio of about 1:10 (about 100-300 μg/mL) in different compositions containing either TAE buffer (pH 8), or one or more compounds in compositions with TAE buffer (pH 8) as shown in FIGS. 24 C and D:
M=PCR Marker
1. 1×TAE Buffer (pH 8)
2. 50% DMSO+300 mM Glucose+500 mM Choline-Cl+400 mM DMSP+1×TAE (pH 8)
3. 12.5 mg/mL K—PO4 Starch 250F+200 mM Quinolinic Acid (pH 5-7)+500 mM L-Carnitine+500 mM DMSP+1×TAE (pH 8)
4. 500 mM Sucrose+1M Choline-Cl+100 mM HMP+10 mg/mL PEG-PSBMA (PEG ˜5 kDa; PSBMA ˜13 kDa)+400 mM Ectoine+200 mM DMSP+1×TAE (pH 8)
5. 500 mM Sorbitol+100 mM Na-Benzoate+500 mM Choline-Cl+25 mg/mL Na—PO4 Sorbitol 280F+25 mg/mL Cationic-Sorbitol 300F+750 mM TMG+50 mM Taurine+400 mM DMSP+1×TAE (pH 8)
6. −80° C.

Following dilution of each sample in each respective RNA storage environment, samples were stored in a thermal cycler at 60° C. for about 48 hours and then analyzed by denaturing agarose gel electrophoresis as shown in FIGS. 24 C and D.
PEG-PSBMA was (PEG Mn 5,000; PSBMA Mn 13,000)

### Example 53

### Accelerated RNA Stability Testing at 60° C.

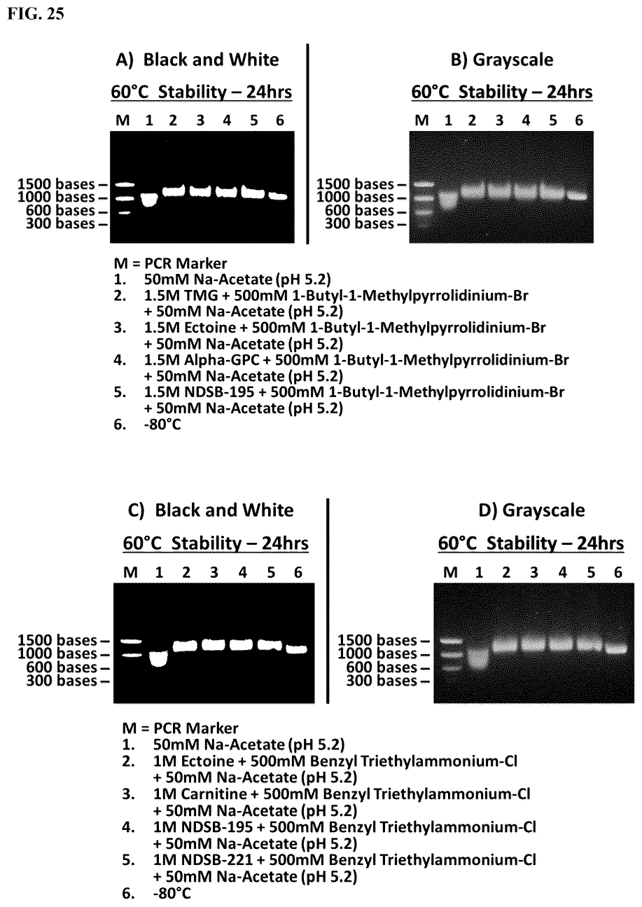

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 25A and B:
M=PCR Marker
1. 50 mM Na-Acetate (pH 5.2)
2. 1.5M TMG+500 mM 1-Butyl-1-Methylpyrrolidinium-Br+50 mM Na-Acetate (pH 5.2)
3. 1.5M Ectoine+500 mM 1-Butyl-1-Methylpyrrolidinium-Br+50 mM Na-Acetate (pH 5.2)
4. 1.5M Alpha-GPC+500 mM 1-Butyl-1-Methylpyrrolidinium-Br+50 mM Na-Acetate (pH 5.2)
5. 1.5M NDSB-195+500 mM 1-Butyl-1-Methylpyrrolidinium-Br+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 25A and B.

### Example 54

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 25 C and D.
M=PCR Marker
1. 50 mM Na-Acetate (pH 5.2)
2. 1M Ectoine+500 mM Benzyl Triethylammonium-Cl+50 mM Na-Acetate (pH 5.2)
3. 1M L-Carnitine+500 mM Benzyl Triethylammonium-Cl+50 mM Na-Acetate (pH 5.2)
4. 1M NDSB-195+500 mM Benzyl Triethylammonium-Cl+50 mM Na-Acetate (pH 5.2)
5. 1M NDSB-221+500 mM Benzyl Triethylammonium-Cl+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 25 C and D.

### Example 55

### Accelerated RNA Stability Testing at 60° C.

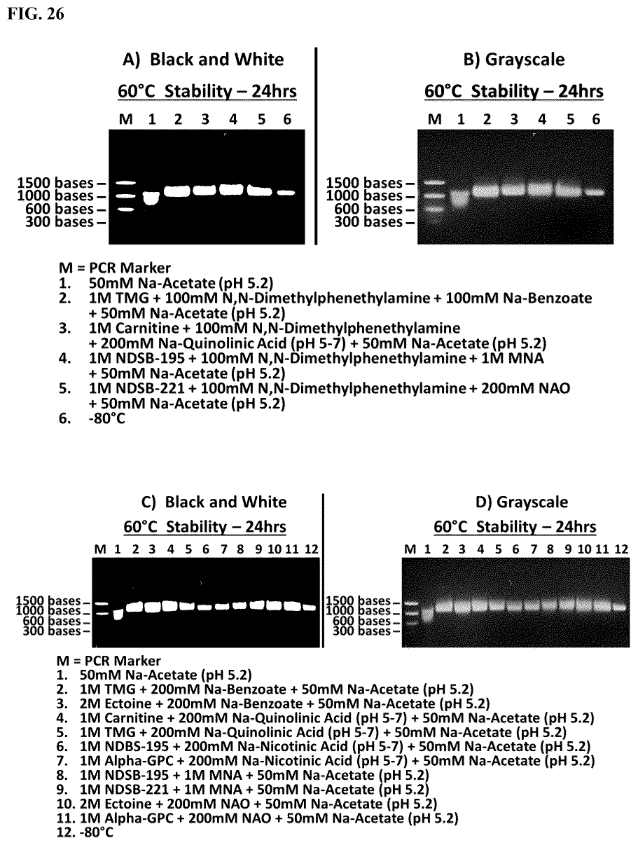

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 26A and B:
M=PCR Marker
1. 50 mM Na-Acetate (pH 5.2)
2. 1M TMG+100 mM N,N-Dimethylphenethylamine+100 mM Na-Benzoate+50 mM Na-Acetate (pH 5.2)
3. 1M L-Carnitine+100 mM N,N-Dimethylphenethylamine+200 mM Na-Quinolinic Acid (pH 5-7)+50 mM Na-Acetate (pH 5.2)
4. 1M NDSB-195+100 mM N,N-Dimethylphenethylamine+1M MNA+50 mM Na-Acetate (pH 5.2)
5. 1M NDSB-221+100 mM N,N-Dimethylphenethylamine+200 mM NAO+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 26A and B.
Abbreviations: 1-Methylnicotinamide-Cl (MNA) and Nicotinamide N-oxide (NAO)

### Example 56

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 26 C and D:
M=PCR Marker
1. 50 mM Na-Acetate (pH 5.2)
2. 1M TMG+200 mM Na-Benzoate+50 mM Na-Acetate (pH 5.2)
3. 2M Ectoine+200 mM Na-Benzoate+50 mM Na-Acetate (pH 5.2)
4. 1M Carnitine+200 mM Na-Quinolinic Acid (pH 5-7)+50 mM Na-Acetate (pH 5.2)
5. 1M TMG+200 mM Na-Quinolinic Acid (pH 5-7)+50 mM Na-Acetate (pH 5.2)
6. 1M NDBS-195+200 mM Na-Nicotinic Acid (pH 5-7)+50 mM Na-Acetate (pH 5.2)
7. 1M Alpha-GPC+200 mM Na-Nicotinic Acid (pH 5-7)+50 mM Na-Acetate (pH 5.2)
8. 1M NDSB-195+1M MNA+50 mM Na-Acetate (pH 5.2)
9. 1M NDSB-221+1M MNA+50 mM Na-Acetate (pH 5.2)
10. 2M Ectoine+200 mM NAO+50 mM Na-Acetate (pH 5.2)
11. 1M Alpha-GPC+200 mM NAO+50 mM Na-Acetate (pH 5.2)
12. −80° C.

Following dilution of each sample in each respective RNA storage environment, samples were stored in a thermal cycler at 60° C. for about 24 hours and then analyzed by denaturing agarose gel electrophoresis as shown in FIGS. 26 C and D.
Abbreviations: 1-Methylnicotinamide-Cl (MNA) and Nicotinamide N-oxide (NAO)

### Example 57

### Accelerated RNA Stability Testing at 60° C.

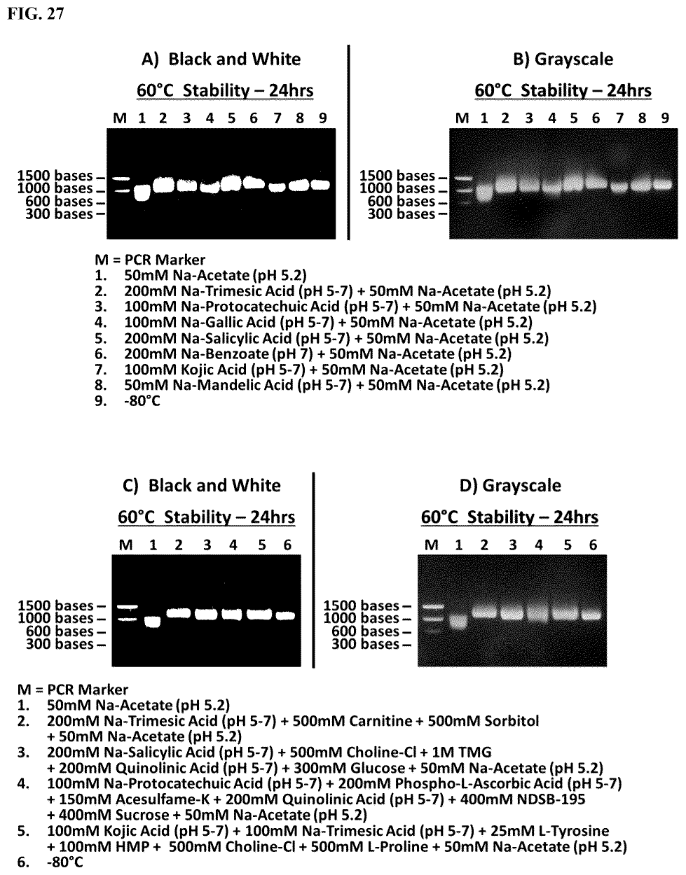

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 27A and B:
M=PCR Marker
1. 50 mM Na-Acetate (pH 5.2)
2. 200 mM Na-Trimesic Acid (pH 5-7)+50 mM Na-Acetate (pH 5.2)
3. 100 mM Na-Protocatechuic Acid (pH 5-7)+50 mM Na-Acetate (pH 5.2)
4. 100 mM Na-Gallic Acid (pH 5-7)+50 mM Na-Acetate (pH 5.2)
5. 200 mM Na-Salicylic Acid (pH 5-7)+50 mM Na-Acetate (pH 5.2)
6. 200 mM Na-Benzoate (pH 7)+50 mM Na-Acetate (pH 5.2)
7. 100 mM Kojic Acid (pH 5-7)+50 mM Na-Acetate (pH 5.2)
8. 50 mM Na-Mandelic Acid (pH 5-7)+50 mM Na-Acetate (pH 5.2)
9. −80° C.

Following dilution of each sample in each respective RNA storage environment, samples were stored in a thermal cycler at 60° C. for about 24 hours and then analyzed by denaturing agarose gel electrophoresis as shown in FIGS. 27A and B.

Prior to analysis by agarose gel electrophoresis, sample 5 was buffer exchanged to remove Na-Salicylic Acid to help improve analysis by gel electrophoresis.

The following were purchased from: Trimesic Acid (Cayman Chemical, Product #19198), Protocatechuic Acid (Cayman Chemical, Product #14916), Gallic Acid (Cayman Chemical, Product #11846), Salicylic Acid (Talsen Chemicals, Richmond Hill, NY), Kojic Acid (Talsen Chemicals), Mandelic Acid (Talsen Chemicals).

### Example 58

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 27 C and D:
M=PCR Marker
1. 50 mM Na-Acetate (pH 5.2)
2. 200 mM Na-Trimesic Acid (pH 5-7)+500 mM L-Carnitine+500 mM Sorbitol+50 mM Na-Acetate (pH 5.2)
3. 200 mM Na-Salicylic Acid (pH 5-7)+500 mM Choline-Cl+1M TMG+200 mM Quinolinic Acid (pH 5-7)+300 mM Glucose+50 mM Na-Acetate (pH 5.2)
4. 100 mM Na-Protocatechuic Acid (pH 5-7)+200 mM Phospho-L-Ascorbic Acid (pH 5-7)+150 mM Acesulfame-K+200 mM Quinolinic Acid (pH 5-7)+400 mM NDSB-195+400 mM Sucrose+50 mM Na-Acetate (pH 5.2)
5. 100 mM Kojic Acid (pH 5-7)+100 mM Na-Trimesic Acid (pH 5-7)+25 mM L-Tyrosine+100 mM HMP+500 mM Choline-Cl+500 mM L-Proline+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 27 C and D.

Prior to analysis by agarose gel electrophoresis, samples 3 and 4 were buffer exchanged to remove Na-Salicylic Acid and Na-Protocatechuic Acid to help improve analysis by gel electrophoresis.

The following were purchased from: L-Tyrosine (PureBulk, Roseburg, OR), Acesulfame-K (Cayman Chemical, Product #33356), 2-Phospho-L-ascorbic acid (Sigma-Aldrich, Product #49752).

### Example 59

### Accelerated RNA Stability Testing at 60° C.

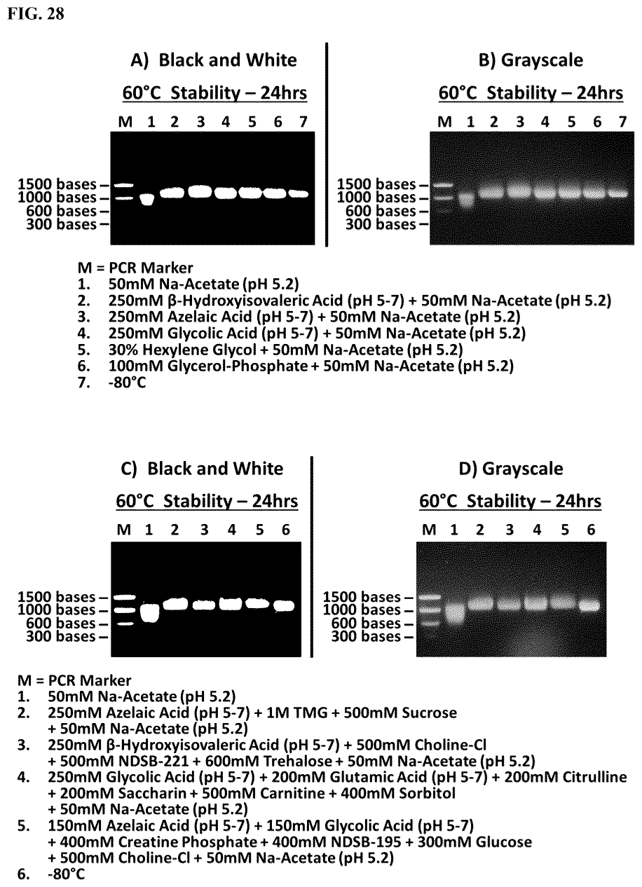

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 28A and B.
M=PCR Marker
1. 50 mM Na-Acetate (pH 5.2)
2. 250 mM B-Hydroxyisovaleric Acid (pH 5-7)+50 mM Na-Acetate (pH 5.2)
3. 250 mM Azelaic Acid (pH 5-7)+50 mM Na-Acetate (pH 5.2)
4. 250 mM Glycolic Acid (pH 5-7)+50 mM Na-Acetate (pH 5.2)
5. 30% Hexylene Glycol+50 mM Na-Acetate (pH 5.2)
6. 100 mM Glycerol-Phosphate+50 mM Na-Acetate (pH 5.2)
7. −80° C.

Following dilution of each sample in each respective RNA storage environment, samples were stored in a thermal cycler at 60° C. for about 24 hours and then analyzed by denaturing agarose gel electrophoresis as shown in FIGS. 28A and B.

The following were purchased from: beta-Hydroxyisovaleric Acid (Cayman Chemical, Product #34030), Azelaic Acid (Talsen Chemicals), Glycolic Acid (Talsen Chemicals).

### Example 60

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 28 C and D:
M=PCR Marker
1. 50 mM Na-Acetate (pH 5.2)
2. 250 mM Azelaic Acid (pH 5-7)+1M TMG+500 mM Sucrose+50 mM Na-Acetate (pH 5.2)
3. 250 mM B-Hydroxyisovaleric Acid (pH 5-7)+500 mM Choline-Cl+500 mM NDSB-221+600 mM Trehalose+50 mM Na-Acetate (pH 5.2)
4. 250 mM Glycolic Acid (pH 5-7)+200 mM Glutamic Acid (pH 5-7)+200 mM Citrulline+200 mM Saccharin+500 mM L-Carnitine+400 mM Sorbitol+50 mM Na-Acetate (pH 5.2)
5. 150 mM Azelaic Acid (pH 5-7)+150 mM Glycolic Acid (pH 5-7)+400 mM Creatine Phosphate+400 mM NDSB-195+300 mM Glucose+500 mM Choline-Cl+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 28 C and D.

The following were purchased from: Glutamic Acid (purchased as Monosodium Glutamate) (Modernist Pantry, Eliot, ME, Product #1594-50), Citrulline (Cayman Chemical, Product #35648), Na-Saccharin (Eisen-Golden, Dublin, CA), Creatine Phosphate (Cayman Chemical, Product #37803).

### Example 61

### Accelerated RNA Stability Testing at 60° C.

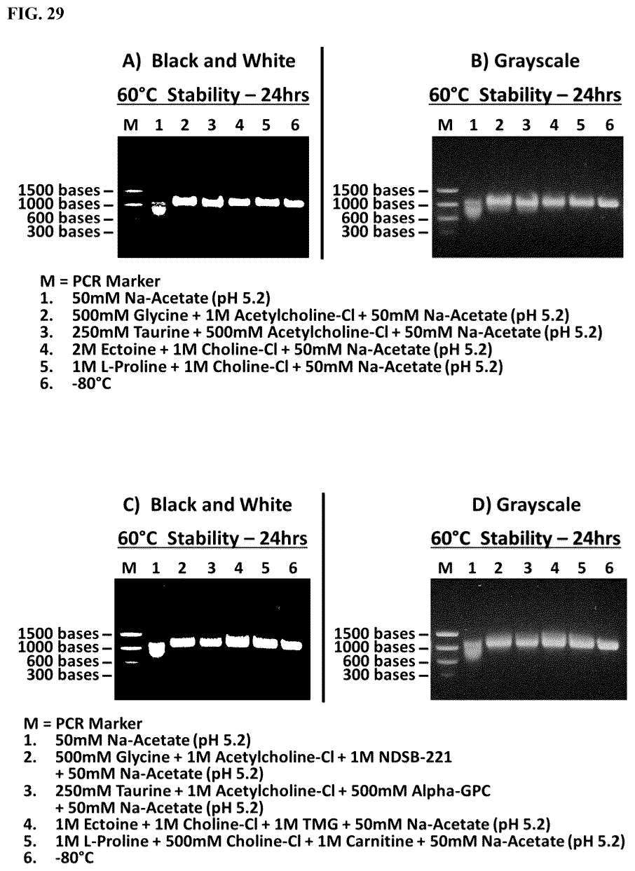

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 29A and B.
M=PCR Marker
1. 50 mM Na-Acetate (pH 5.2)
2. 500 mM Glycine+1M Acetylcholine-Cl+50 mM Na-Acetate (pH 5.2)
3. 250 mM Taurine+500 mM Acetylcholine-Cl+50 mM Na-Acetate (pH 5.2)
4. 2M Ectoine+1M Choline-Cl+50 mM Na-Acetate (pH 5.2)
5. 1M L-Proline+1M Choline-Cl+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 29A and B.

### Example 62

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 29 C and D:
M=PCR Marker
1. 50 mM Na-Acetate (pH 5.2)
2. 500 mM Glycine+1M Acetylcholine-Cl+1M NDSB-221+50 mM Na-Acetate (pH 5.2)
3. 250 mM Taurine+1M Acetylcholine-Cl+500 mM Alpha-GPC+50 mM Na-Acetate (pH 5.2)
4. 1M Ectoine+1M Choline-Cl+1M TMG+50 mM Na-Acetate (pH 5.2)
5. 1M L-Proline+500 mM Choline-Cl+1M L-Carnitine+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 29 C and D.

### Example 63

### Accelerated RNA Stability Testing at 60° C.

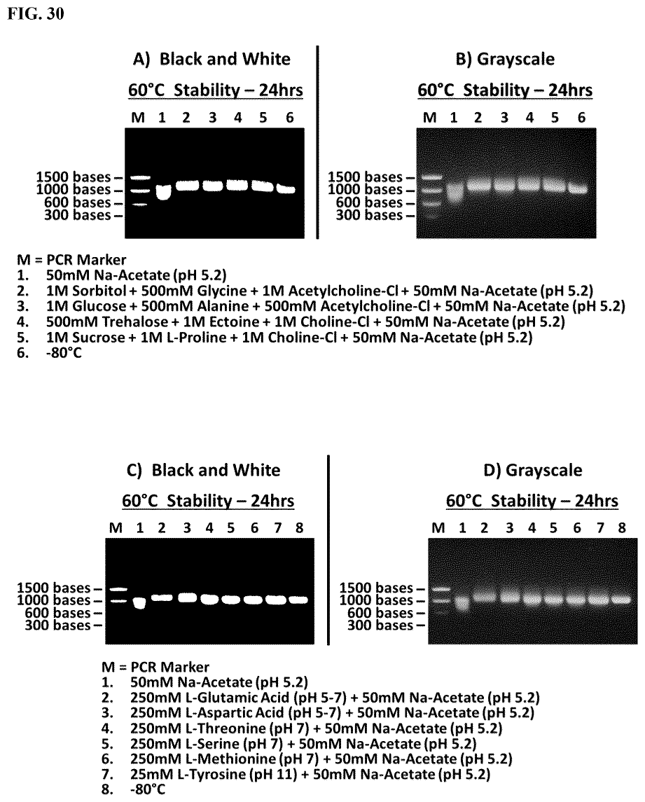

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 30A and B:
M=PCR Marker
1. 50 mM Na-Acetate (pH 5.2)
2. 1M Sorbitol+500 mM Glycine+1M Acetylcholine-Cl+50 mM Na-Acetate (pH 5.2)
3. 1M Glucose+500 mM L-Alanine+500 mM Acetylcholine-Cl+50 mM Na-Acetate (pH 5.2)
4. 500 mM Trehalose+1M Ectoine+1M Choline-Cl+50 mM Na-Acetate (pH 5.2)
5. 1M Sucrose+1M L-Proline+1M Choline-Cl+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 30A and B.

### Example 64

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 30 C and D.
M=PCR Marker
1. 50 mM Na-Acetate (pH 5.2)
2. 250 mM L-Glutamic Acid (pH 5-7)+50 mM Na-Acetate (pH 5.2)
3. 250 mM L-Aspartic Acid (pH 5-7)+50 mM Na-Acetate (pH 5.2)
4. 250 mM L-Threonine (pH 7)+50 mM Na-Acetate (pH 5.2)
5. 250 mM L-Serine (pH 7)+50 mM Na-Acetate (pH 5.2)
6. 250 mM L-Methionine (pH 7)+50 mM Na-Acetate (pH 5.2)
7. 25 mM L-Tyrosine (pH 11)+50 mM Na-Acetate (pH 5.2)
8. −80° C.

Following dilution of each sample in each respective RNA storage environment, samples were stored in a thermal cycler at 60° C. for about 24 hours and then analyzed by denaturing agarose gel electrophoresis as shown in FIGS. 30 C and D.

L-Tyrosine pH was adjusted to 11 to increase solubility and then added to sample. Some precipitation occurred following addition to 50 mM Acetate (pH 5.2)

The following were purchased from: L-Aspartic Acid (purchased as Potassium-L-Aspartate) (PureBulk, Roseburg, OR), L-Threonine (PureBulk), L-Serine (PureBulk), L-Methionine (PureBulk), L-Tyrosine (PureBulk).

### Example 65

### Accelerated RNA Stability Testing at 60° C.

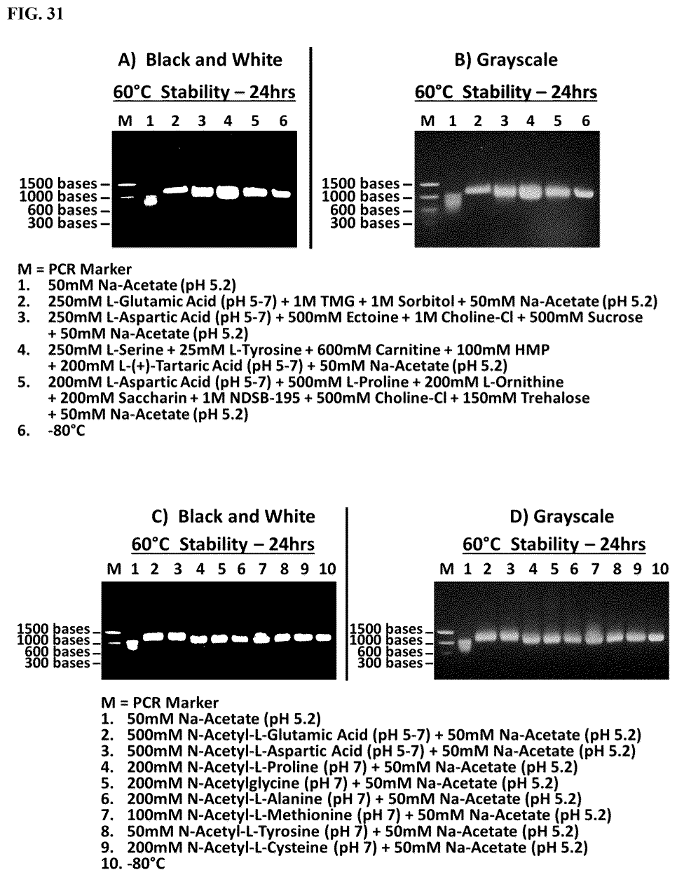

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 31A and B.
M=PCR Marker
1. 50 mM Na-Acetate (pH 5.2)
2. 250 mM L-Glutamic Acid (pH 5-7)+1M TMG+1M Sorbitol+50 mM Na-Acetate (pH 5.2)
3. 250 mM L-Aspartic Acid (pH 5-7)+500 mM Ectoine+1M Choline-Cl+500 mM Sucrose+50 mM Na-Acetate (pH 5.2)
4. 250 mM L-Serine+25 mM L-Tyrosine+600 mM L-Carnitine+100 mM HMP+200 mM L-(+)-Tartaric Acid (pH 5-7)+50 mM Na-Acetate (pH 5.2)
5. 200 mM L-Aspartic Acid (pH 5-7)+500 mM L-Proline+200 mM L-Ornithine+200 mM Saccharin+1M NDSB-195+500 mM Choline-Cl+150 mM Trehalose+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 31A and B.

The following were purchased from: L-Ornithine (Sigma-Aldrich, Product #02375)

### Example 66

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 31 C and D:
M=PCR Marker
1. 50 mM Na-Acetate (pH 5.2)
2. 500 mM N-Acetyl-L-Glutamic Acid (pH 5-7)+50 mM Na-Acetate (pH 5.2)
3. 500 mM N-Acetyl-L-Aspartic Acid (pH 5-7)+50 mM Na-Acetate (pH 5.2)
4. 200 mM N-Acetyl-L-Proline (pH 7)+50 mM Na-Acetate (pH 5.2)
5. 200 mM N-Acetylglycine (pH 7)+50 mM Na-Acetate (pH 5.2)
6. 200 mM N-Acetyl-L-Alanine (pH 7)+50 mM Na-Acetate (pH 5.2)
7. 100 mM N-Acetyl-L-Methionine (pH 7)+50 mM Na-Acetate (pH 5.2)
8. 50 mM N-Acetyl-L-Tyrosine (pH 7)+50 mM Na-Acetate (pH 5.2)
9. 200 mM N-Acetyl-L-Cysteine (pH 7)+50 mM Na-Acetate (pH 5.2)
10. −80° C.

Following dilution of each sample in each respective RNA storage environment, samples were stored in a thermal cycler at 60° C. for about 24 hours and then analyzed by denaturing agarose gel electrophoresis as shown in FIGS. 31 C and D.

Prior to analysis by agarose gel electrophoresis, sample 8 was buffer exchanged to remove N-Acetyl-L-Tyrosine to help improve analysis by gel electrophoresis.

The following were purchased from: N-Acetyl-L-Glutamic Acid (Sigma-Aldrich, Product #855642), N-Acetyl-L-Aspartic Acid (Cayman Chemical, Product #34635), N-Acetyl-L-Proline (Sigma-Aldrich, Product #A0783), N-Acetylglycine (Sigma-Aldrich, Product #A16300), N-Acetyl-L-Alanine (Sigma-Aldrich, Product #A4625), N-Acetyl-L-Methionine (Sigma-Aldrich, Product #01310), N-Acetyl-L-Tyrosine (PureBulk), N-Acetyl-L-Cysteine (Sigma Aldrich, Product #A9165).

### Example 67

### Accelerated RNA Stability Testing at 60° C.

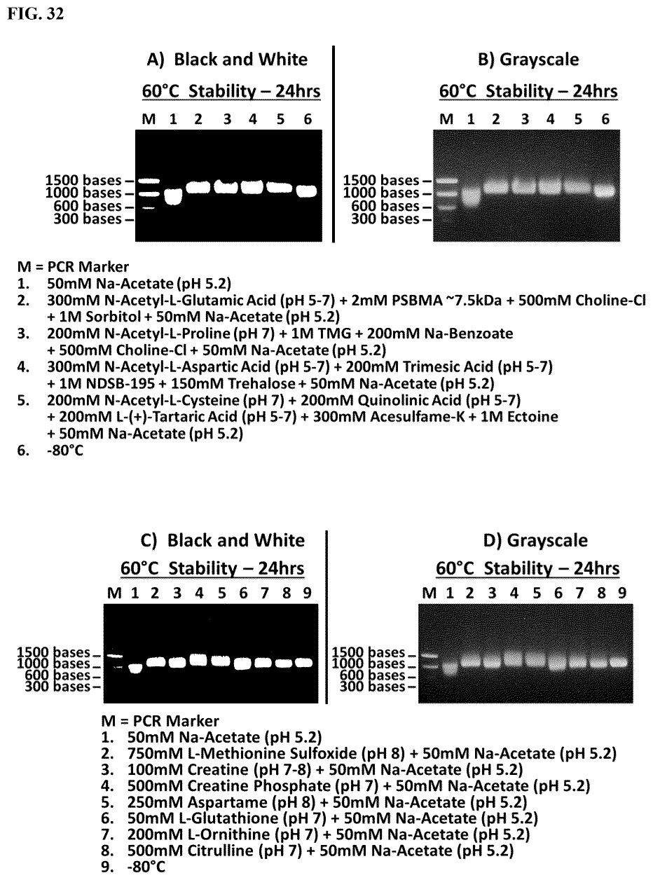

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 32A and B.
M=PCR Marker
1. 50 mM Na-Acetate (pH 5.2)
2. 300 mM N-Acetyl-L-Glutamic Acid (pH 5-7)+2 mM PSBMA ˜7.5 kDa+500 mM Choline-Cl+1M Sorbitol+50 mM Na-Acetate (pH 5.2)
3. 200 mM N-Acetyl-L-Proline (pH 7)+1M TMG+200 mM Na-Benzoate+500 mM Choline-Cl+50 mM Na-Acetate (pH 5.2)
4. 300 mM N-Acetyl-L-Aspartic Acid (pH 5-7)+200 mM Trimesic Acid (pH 5-7)+1M NDSB-195+150 mM Trehalose+50 mM Na-Acetate (pH 5.2)
5. 200 mM N-Acetyl-L-Cysteine (pH 7)+200 mM Quinolinic Acid (pH 5-7)+200 mM L-(+)-Tartaric Acid (pH 5-7)+300 mM Acesulfame-K+1M Ectoine+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 32A and B.

### Example 68

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 32 C and D:
M=PCR Marker
1. 50 mM Na-Acetate (pH 5.2)
2. 750 mM L-Methionine Sulfoxide (pH 8)+50 mM Na-Acetate (pH 5.2)
3. 100 mM Creatine (pH 7-8)+50 mM Na-Acetate (pH 5.2)
4. 500 mM Creatine Phosphate (pH 7)+50 mM Na-Acetate (pH 5.2)
5. 250 mM Aspartame (pH 8)+50 mM Na-Acetate (pH 5.2)
6. 50 mM L-Glutathione (pH 7)+50 mM Na-Acetate (pH 5.2)
7. 200 mM L-Ornithine (pH 7)+50 mM Na-Acetate (pH 5.2)
8. 500 mM Citrulline (pH 7)+50 mM Na-Acetate (pH 5.2)
9. −80° C.

Following dilution of each sample in each respective RNA storage environment, samples were stored in a thermal cycler at 60° C. for about 24 hours and then analyzed by denaturing agarose gel electrophoresis as shown in FIGS. 32 C and D.

Aspartame was adjusted to pH 8 to improve solubility prior to adding to sample.

The following were purchased from: L-Methionine Sulfoxide (Cayman Chemical, Product #36255), L-Glutathione (Cayman Chemical, Product #35825), Aspartame (Cayman Chemical, Product #26089), Creatine (Myogenix, Maria, CA).

### Example 69

### Accelerated RNA Stability Testing at 60° C.

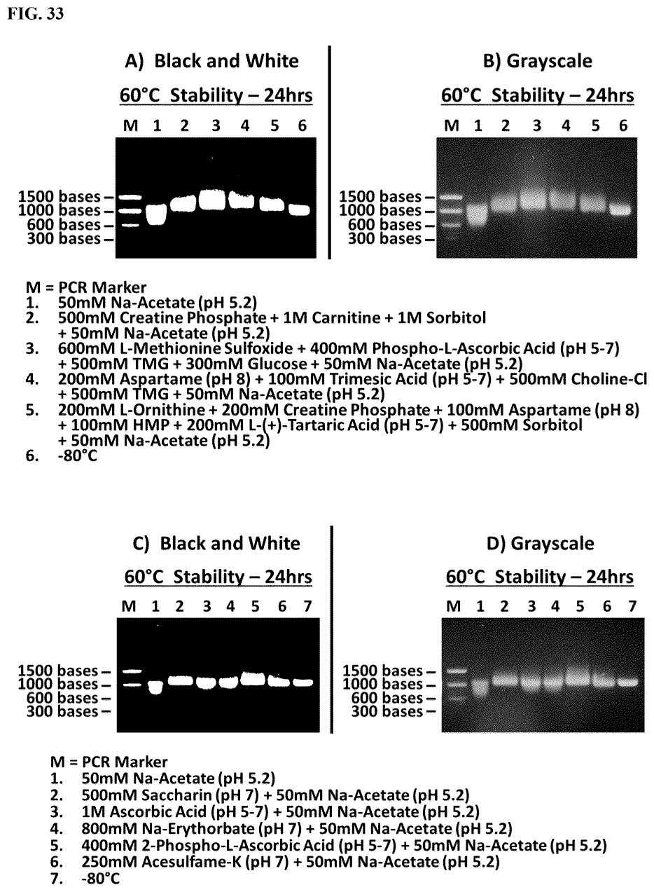

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 33A and B:
M=PCR Marker
1. 50 mM Na-Acetate (pH 5.2)
2. 500 mM Creatine Phosphate+1M Carnitine+1M Sorbitol+50 mM Na-Acetate (pH 5.2)
3. 600 mM L-Methionine Sulfoxide+400 mM Phospho-L-Ascorbic Acid (pH 5-7)+500 mM TMG+300 mM Glucose+50 mM Na-Acetate (pH 5.2)
4. 200 mM Aspartame (pH 8)+100 mM Trimesic Acid (pH 5-7)+500 mM Choline-Cl+500 mM TMG+50 mM Na-Acetate (pH 5.2)
5. 200 mM L-Ornithine+200 mM Creatine Phosphate+100 mM Aspartame (pH 8)+100 mM HMP+200 mM L-(+)-Tartaric Acid (pH 5-7)+500 mM Sorbitol+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 33A and B.

### Example 70

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 33 C and D:
M=PCR Marker
1. 50 mM Na-Acetate (pH 5.2)
2. 500 mM Saccharin (pH 7)+50 mM Na-Acetate (pH 5.2)
3. 1M Ascorbic Acid (pH 5-7)+50 mM Na-Acetate (pH 5.2)
4. 800 mM Na-Erythorbate (pH 7)+50 mM Na-Acetate (pH 5.2)
5. 400 mM 2-Phospho-L-Ascorbic Acid (pH 5-7)+50 mM Na-Acetate (pH 5.2)
6. 250 mM Acesulfame-K (pH 7)+50 mM Na-Acetate (pH 5.2)
7. −80° C.

Following dilution of each sample in each respective RNA storage environment, samples were stored in a thermal cycler at 60° C. for about 24 hours and then analyzed by denaturing agarose gel electrophoresis as shown in FIGS. 33 C and D.

Ascorbic Acid and Na-Erythorbate were made fresh prior to adding to sample.

The following were purchased from: Ascorbic Acid (Eisen-Golden), Na-Erythorbate (Eisen-Golden).

### Example 71

### Accelerated RNA Stability Testing at 60° C.

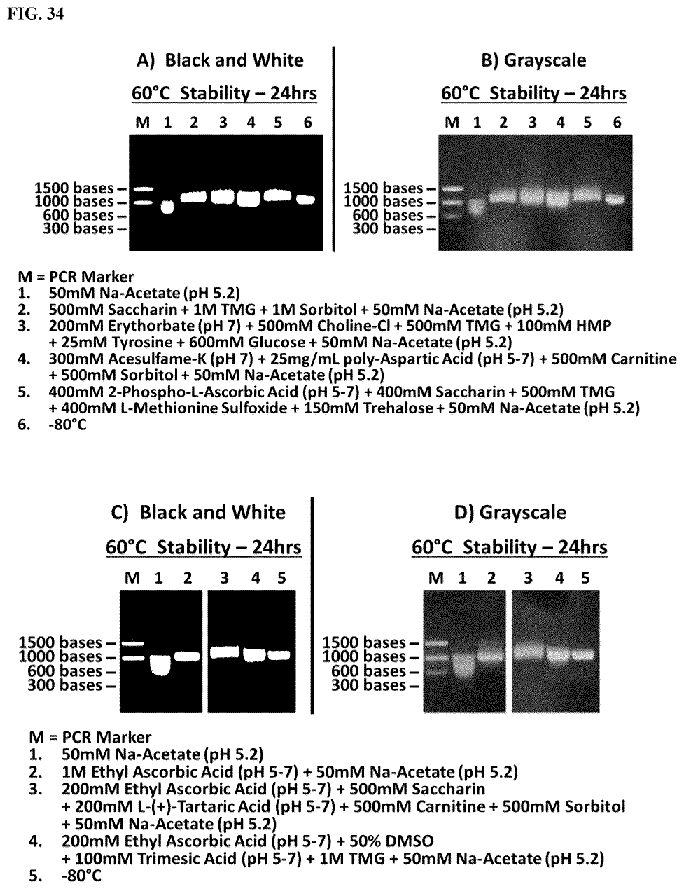

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 34A and B:
M=PCR Marker
1. 50 mM Na-Acetate (pH 5.2)
2. 500 mM Saccharin+1M TMG+1M Sorbitol+50 mM Na-Acetate (pH 5.2)
3. 200 mM Erythorbate (pH 7)+500 mM Choline-Cl+500 mM TMG+100 mM HMP+25 mM L-Tyrosine+600 mM Glucose+50 mM Na-Acetate (pH 5.2)
4. 300 mM Acesulfame-K (pH 7)+25 mg/mL poly-Aspartic Acid (pH 5-7)+500 mM L-Carnitine+500 mM Sorbitol+50 mM Na-Acetate (pH 5.2)
5. 400 mM 2-Phospho-L-Ascorbic Acid (pH 5-7)+400 mM Saccharin+500 mM TMG+400 mM L-Methionine Sulfoxide+150 mM Trehalose+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 34A and B.

Ascorbic Acid and Na-Erythorbate were made fresh prior to adding to sample.

The following were purchased from: poly-Aspartic Acid (Mark Nature, Fullerton, CA).

### Example 72

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 34 C and D:
M=PCR Marker
1. 50 mM Na-Acetate (pH 5.2)
2. 1M Ethyl Ascorbic Acid (pH 5-7)+50 mM Na-Acetate (pH 5.2)
3. 200 mM Ethyl Ascorbic Acid (pH 5-7)+500 mM Saccharin+200 mM L-(+)-Tartaric Acid (pH 5-7)+500 mM Carnitine+500 mM Sorbitol+50 mM Na-Acetate (pH 5.2)
4. 200 mM Ethyl Ascorbic Acid (pH 5-7)+50% DMSO+100 mM Trimesic Acid (pH 5-7)+1M TMG+50 mM Na-Acetate (pH 5.2)
5. −80° C.

Following dilution of each sample in each respective RNA storage environment, samples were stored in a thermal cycler at 60° C. for about 24 hours and then analyzed by denaturing agarose gel electrophoresis as shown in FIGS. 34 C and D.

Ethyl Ascorbic Acid was made fresh prior to adding to sample.

The following were purchased from: Ethyl Ascorbic Acid (Pure Health Botanicals, LLC, Saint Charles, IL, dba as ebay purehealthsolutions), L-(+)-Tartaric Acid (Sigma Aldrich, Product #251380).

### Example 73

### Accelerated RNA Stability Testing at 60° C.

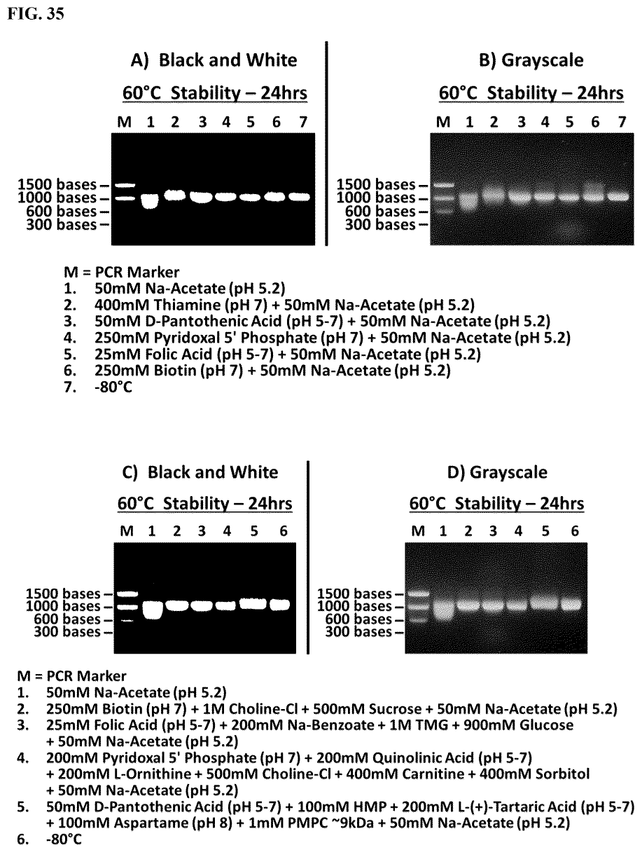

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 35A and B:
M=PCR Marker
1. 50 mM Na-Acetate (pH 5.2)
2. 400 mM Thiamine (pH 7)+50 mM Na-Acetate (pH 5.2)
3. 50 mM D-Pantothenic Acid (pH 5-7)+50 mM Na-Acetate (pH 5.2)
4. 250 mM Pyridoxal 5′ Phosphate (pH 7)+50 mM Na-Acetate (pH 5.2)
5. 25 mM Folic Acid (pH 5-7)+50 mM Na-Acetate (pH 5.2)
6. 250 mM Biotin (pH 7)+50 mM Na-Acetate (pH 5.2)
7. −80° C.

Following dilution of each sample in each respective RNA storage environment, samples were stored in a thermal cycler at 60° C. for about 24 hours and then analyzed by denaturing agarose gel electrophoresis as shown in FIGS. 35A and B.

Prior to analysis by agarose gel electrophoresis, samples 5 and 6 were buffer exchanged to remove folic acid and biotin to help improve analysis by gel electrophoresis.

The following were purchased from: Thiamine (Cayman Chemical, Product #25656), D-Pantothenic Acid (Cayman Chemical, Product #17288), Pyridoxal 5′ Phosphate (Cayman Chemical, Product #20352), Folic Acid (Cayman Chemical, Product #20515), Biotin (Cayman Chemical, Product #22582).

### Example 74

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 35 C and D.
M=PCR Marker
1. 50 mM Na-Acetate (pH 5.2)
2. 250 mM Biotin (pH 7)+1M Choline-Cl+500 mM Sucrose+50 mM Na-Acetate (pH 5.2)
3. 25 mM Folic Acid (pH 5-7)+200 mM Na-Benzoate+1M TMG+900 mM Glucose+50 mM Na-Acetate (pH 5.2)
4. 200 mM Pyridoxal 5′ Phosphate (pH 7)+200 mM Quinolinic Acid (pH 5-7)+200 mM L-Ornithine+500 mM Choline-Cl+400 mM Carnitine+400 mM Sorbitol+50 mM Na-Acetate (pH 5.2)
5. 50 mM D-Pantothenic Acid (pH 5-7)+100 mM HMP+200 mM L-(+)-Tartaric Acid (pH 5-7)+100 mM Aspartame (pH 8)+1 mM PMPC ˜9 kDa+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 35 C and D.

Prior to analysis by agarose gel electrophoresis, samples 2 and 3 were buffer exchanged to remove folic acid and biotin to help improve analysis by gel electrophoresis.

### Example 75

### Accelerated RNA Stability Testing at 60° C.

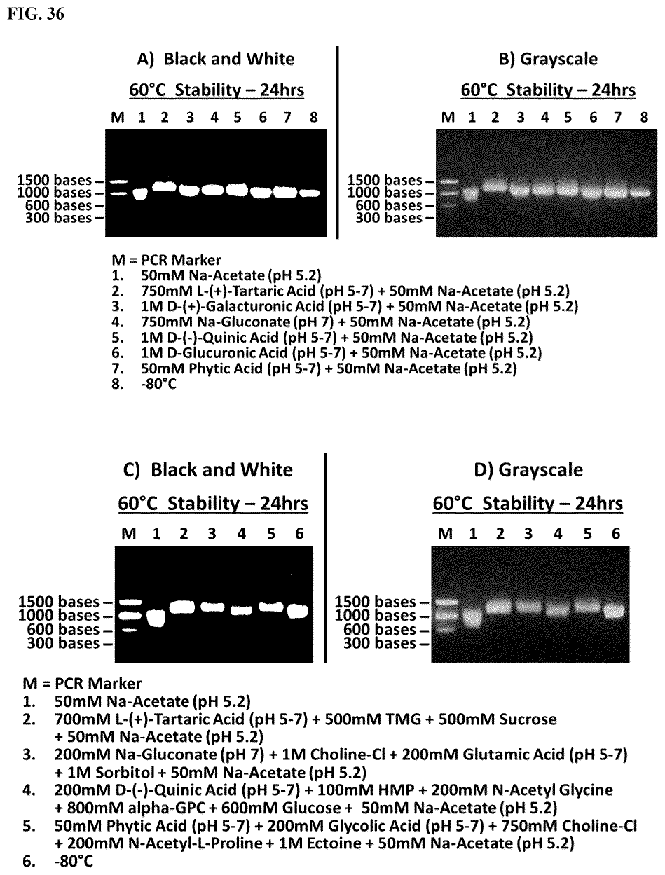

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 36A and B.
M=PCR Marker
1. 50 mM Na-Acetate (pH 5.2)
2. 750 mM L-(+)-Tartaric Acid (pH 5-7)+50 mM Na-Acetate (pH 5.2)
3. 1M D-(+)-Galacturonic Acid (pH 5-7)+50 mM Na-Acetate (pH 5.2)
4. 750 mM Na-Gluconate (pH 7)+50 mM Na-Acetate (pH 5.2)
5. 1M D-(−)-Quinic Acid (pH 5-7)+50 mM Na-Acetate (pH 5.2)
6. 1M D-Glucuronic Acid (pH 5-7)+50 mM Na-Acetate (pH 5.2)
7. 50 mM Phytic Acid (pH 5-7)+50 mM Na-Acetate (pH 5.2)
8. −80° C.

Following dilution of each sample in each respective RNA storage environment, samples were stored in a thermal cycler at 60° C. for about 24 hours and then analyzed by denaturing agarose gel electrophoresis as shown in FIGS. 36A and B.

Prior to analysis by agarose gel electrophoresis, samples 3, 5, and 6 were buffer exchanged to remove galacturonic acid, quinic acid, and glucuronic acid to help improve analysis by gel electrophoresis.

The following were purchased from: D-(+)-Galacturonic acid (Sigma-Aldrich, Product #48280), Na-Gluconate (Sigma Aldrich, Product #S2054), D-(−)-Quinic Acid (Sigma-Aldrich, Product #138622) D-Glucuronic Acid (Sigma-Aldrich, Product #31531).

### Example 76

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 36 C and D.
M=PCR Marker
1. 50 mM Na-Acetate (pH 5.2)
2. 700 mM L-(+)-Tartaric Acid (pH 5-7)+500 mM TMG+500 mM Sucrose+50 mM Na-Acetate (pH 5.2)
3. 200 mM Na-Gluconate (pH 7)+1M Choline-Cl+200 mM Glutamic Acid (pH 5-7)+1M Sorbitol+50 mM Na-Acetate (pH 5.2)
4. 200 mM D-(−)-Quinic Acid (pH 5-7)+100 mM HMP+200 mM N-Acetyl Glycine+800 mM alpha-GPC+600 mM Glucose+50 mM Na-Acetate (pH 5.2)
5. 50 mM Phytic Acid (pH 5-7)+200 mM Glycolic Acid (pH 5-7)+750 mM Choline-Cl+200 mM N-Acetyl-L-Proline+1M Ectoine+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 36 C and D.

### Example 77

### Accelerated RNA Stability Testing at 60° C.

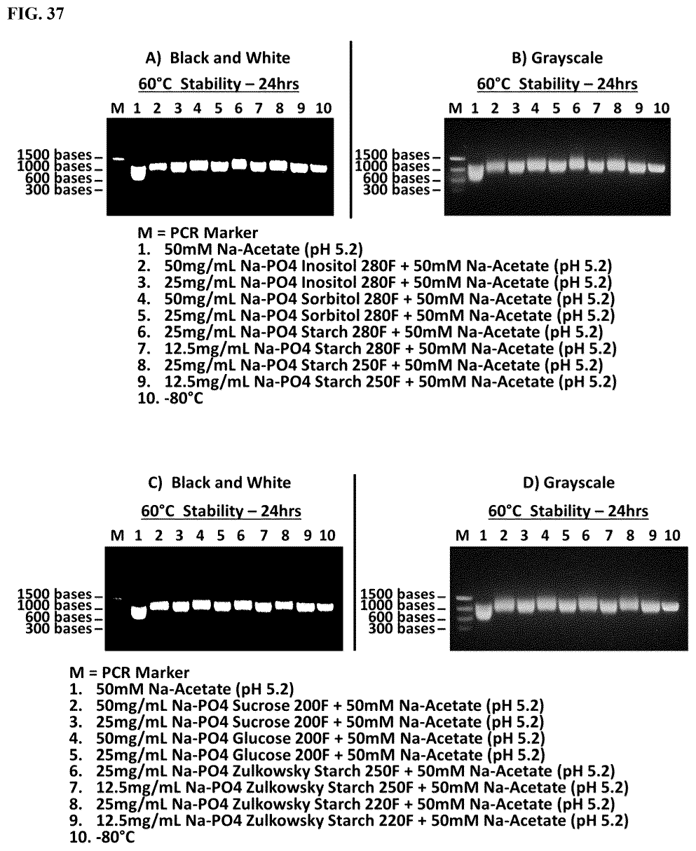

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 37A and B.
M=PCR Marker
1. 50 mM Na-Acetate (pH 5.2)
2. 50 mg/mL Na—PO4 Inositol 280F+50 mM Na-Acetate (pH 5.2)
3. 25 mg/mL Na—PO4 Inositol 280F+50 mM Na-Acetate (pH 5.2)
4. 50 mg/mL Na—PO4 Sorbitol 280F+50 mM Na-Acetate (pH 5.2)
5. 25 mg/mL Na—PO4 Sorbitol 280F+50 mM Na-Acetate (pH 5.2)
6. 25 mg/mL Na—PO4 Starch 280F+50 mM Na-Acetate (pH 5.2)
7. 12.5 mg/mL Na—PO4 Starch 280F+50 mM Na-Acetate (pH 5.2)
8. 25 mg/mL Na—PO4 Starch 250F+50 mM Na-Acetate (pH 5.2)
9. 12.5 mg/mL Na—PO4 Starch 250F+50 mM Na-Acetate (pH 5.2)
10. −80° C.

Following dilution of each sample in each respective RNA storage environment, samples were stored in a thermal cycler at 60° C. for about 24 hours and then analyzed by denaturing agarose gel electrophoresis as shown in FIGS. 37A and B.

### Example 78

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 37 C and D.
M=PCR Marker
1. 50 mM Na-Acetate (pH 5.2)
2. 50 mg/mL Na—PO4 Sucrose 200F+50 mM Na-Acetate (pH 5.2)
3. 25 mg/mL Na—PO4 Sucrose 200F+50 mM Na-Acetate (pH 5.2)
4. 50 mg/mL Na—PO4 Glucose 200F+50 mM Na-Acetate (pH 5.2)
5. 25 mg/mL Na—PO4 Glucose 200F+50 mM Na-Acetate (pH 5.2)
6. 25 mg/mL Na—PO4 Zulkowsky Starch 250F+50 mM Na-Acetate (pH 5.2)
7. 12.5 mg/mL Na—PO4 Zulkowsky Starch 250F+50 mM Na-Acetate (pH 5.2)
8. 25 mg/mL Na—PO4 Zulkowsky Starch 220F+50 mM Na-Acetate (pH 5.2)
9. 12.5 mg/mL Na—PO4 Zulkowsky Starch 220F+50 mM Na-Acetate (pH 5.2)
10. −80° C.

Following dilution of each sample in each respective RNA storage environment, samples were stored in a thermal cycler at 60° C. for about 24 hours and then analyzed by denaturing agarose gel electrophoresis as shown in FIGS. 37 C and D.

### Example 79

### Accelerated RNA Stability Testing at 60° C.

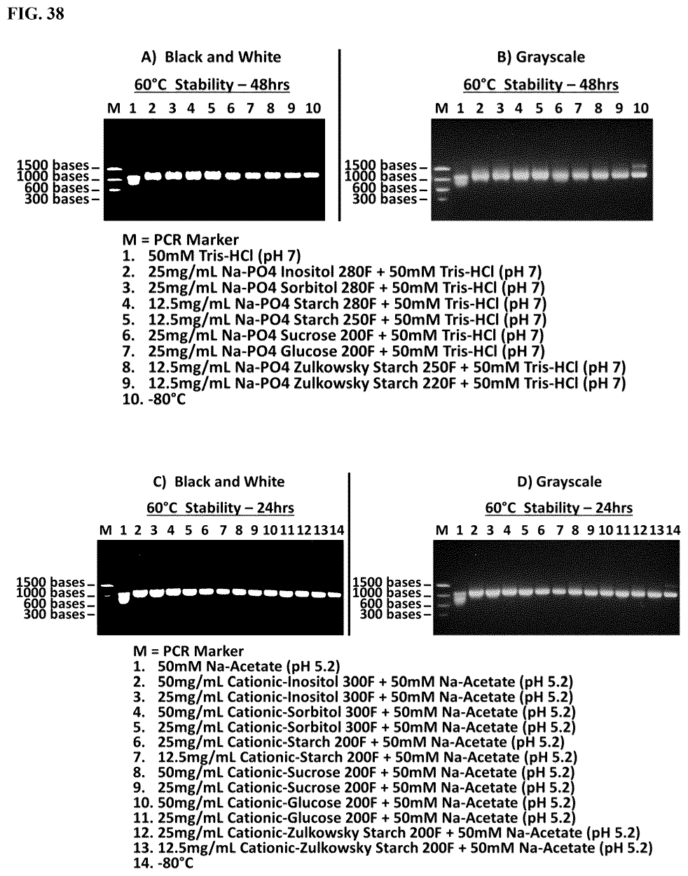

In vitro transcribed RNA was diluted at a ratio of about 1:10 (about 100-300 μg/mL) in different compositions containing either 50 mM Tris-HCl (pH 7), or one or more compounds in compositions with 50 mM Tris-HCl (pH 7) as shown in FIGS. 38A and B.
M=PCR Marker
1. 50 mM Tris-HCl (pH 7)
2. 25 mg/mL Na—PO4 Inositol 280F+50 mM Tris-HCl (pH 7)
3. 25 mg/mL Na—PO4 Sorbitol 280F+50 mM Tris-HCl (pH 7)
4. 12.5 mg/mL Na—PO4 Starch 280F+50 mM Tris-HCl (pH 7)
5. 12.5 mg/mL Na—PO4 Starch 250F+50 mM Tris-HCl (pH 7)
6. 25 mg/mL Na—PO4 Sucrose 200F+50 mM Tris-HCl (pH 7)
7. 25 mg/mL Na—PO4 Glucose 200F+50 mM Tris-HCl (pH 7)
8. 12.5 mg/mL Na—PO4 Zulkowsky Starch 250F+50 mM Tris-HCl (pH 7)
9. 12.5 mg/mL Na—PO4 Zulkowsky Starch 220F+50 mM Tris-HCl (pH 7)
10. −80° C.

Following dilution of each sample in each respective RNA storage environment, samples were stored in a thermal cycler at 60° C. for about 48 hours and then analyzed by denaturing agarose gel electrophoresis as shown in FIGS. 38A and B.

### Example 80

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 38 C and D.
M=PCR Marker
1. 50 mM Na-Acetate (pH 5.2)
2. 50 mg/mL Cationic-Inositol 300F+50 mM Na-Acetate (pH 5.2)
3. 25 mg/mL Cationic-Inositol 300F+50 mM Na-Acetate (pH 5.2)
4. 50 mg/mL Cationic-Sorbitol 300F+50 mM Na-Acetate (pH 5.2)
5. 25 mg/mL Cationic-Sorbitol 300F+50 mM Na-Acetate (pH 5.2)
6. 25 mg/mL Cationic-Starch 200F+50 mM Na-Acetate (pH 5.2)
7. 12.5 mg/mL Cationic-Starch 200F+50 mM Na-Acetate (pH 5.2)
8. 50 mg/mL Cationic-Sucrose 200F+50 mM Na-Acetate (pH 5.2)
9. 25 mg/mL Cationic-Sucrose 200F+50 mM Na-Acetate (pH 5.2)
10. 50 mg/mL Cationic-Glucose 200F+50 mM Na-Acetate (pH 5.2)
11. 25 mg/mL Cationic-Glucose 200F+50 mM Na-Acetate (pH 5.2)
12. 25 mg/mL Cationic-Zulkowsky Starch 200F+50 mM Na-Acetate (pH 5.2)
13. 12.5 mg/mL Cationic-Zulkowsky Starch 200F+50 mM Na-Acetate (pH 5.2)
14. −80° C.

Following dilution of each sample in each respective RNA storage environment, samples were stored in a thermal cycler at 60° C. for about 24 hours and then analyzed by denaturing agarose gel electrophoresis as shown in FIGS. 38 C and D.

### Example 81

### Accelerated RNA Stability Testing at 60° C.

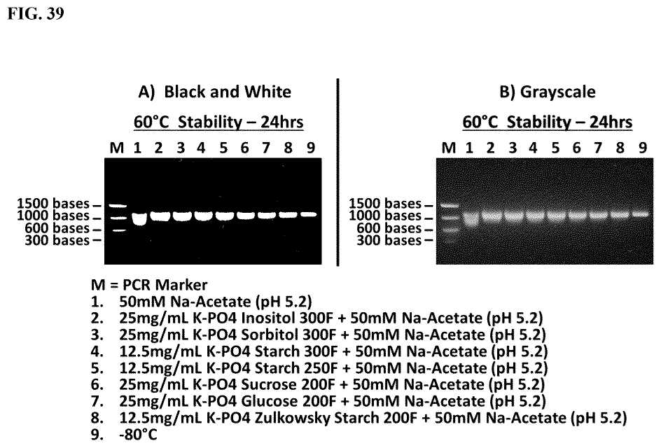

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 39A and B.
M=PCR Marker
1. 50 mM Na-Acetate (pH 5.2)
2. 25 mg/mL K—PO4 Inositol 300F+50 mM Na-Acetate (pH 5.2)
3. 25 mg/mL K—PO4 Sorbitol 300F+50 mM Na-Acetate (pH 5.2)
4. 12.5 mg/mL K—PO4 Starch 300F+50 mM Na-Acetate (pH 5.2)
5. 12.5 mg/mL K—PO4 Starch 250F+50 mM Na-Acetate (pH 5.2)
6. 25 mg/mL K—PO4 Sucrose 200F+50 mM Na-Acetate (pH 5.2)
7. 25 mg/mL K—PO4 Glucose 200F+50 mM Na-Acetate (pH 5.2)
8. 12.5 mg/mL K—PO4 Zulkowsky Starch 200F+50 mM Na-Acetate (pH 5.2)
9. −80° C.

Following dilution of each sample in each respective RNA storage environment, samples were stored in a thermal cycler at 60° C. for about 24 hours and then analyzed by denaturing agarose gel electrophoresis as shown in FIGS. 39A and B.

### Example 82

### Accelerated RNA Stability Testing at 60° C.

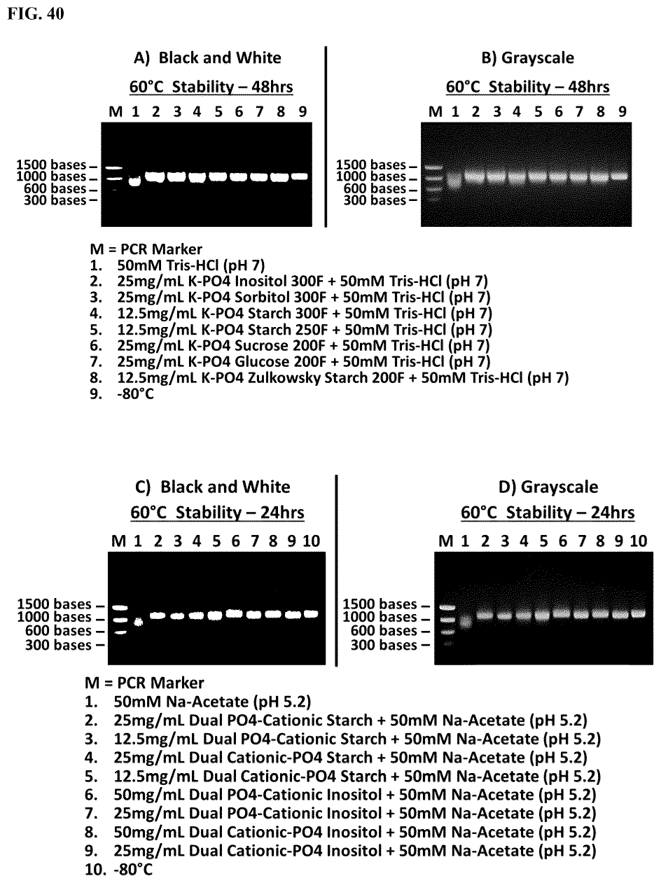

In vitro transcribed RNA was diluted at a ratio of about 1:10 (about 100-300 μg/mL) in different compositions containing either 50 mM Tris-HCl (pH 7), or one or more compounds in compositions with 50 mM Tris-HCl (pH 7) as shown in FIGS. 40A and B.
M=PCR Marker
1. 50 mM Tris-HCl (pH 7)
2. 25 mg/mL K—PO4 Inositol 300F+50 mM Tris-HCl (pH 7)
3. 25 mg/mL K—PO4 Sorbitol 300F+50 mM Tris-HCl (pH 7)
4. 12.5 mg/mL K—PO4 Starch 300F+50 mM Tris-HCl (pH 7)
5. 12.5 mg/mL K—PO4 Starch 250F+50 mM Tris-HCl (pH 7)
6. 25 mg/mL K—PO4 Sucrose 200F+50 mM Tris-HCl (pH 7)
7. 25 mg/mL K—PO4 Glucose 200F+50 mM Tris-HCl (pH 7)
8. 12.5 mg/mL K—PO4 Zulkowsky Starch 200F+50 mM Tris-HCl (pH 7)
9. −80° C.

Following dilution of each sample in each respective RNA storage environment, samples were stored in a thermal cycler at 60° C. for about 48 hours and then analyzed by denaturing agarose gel electrophoresis as shown in FIGS. 40A and B.

### Example 83

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 40 C and D.
M=PCR Marker
1. 50 mM Na-Acetate (pH 5.2)
2. 25 mg/mL Dual PO4-Cationic Starch+50 mM Na-Acetate (pH 5.2)
3. 12.5 mg/mL Dual PO4-Cationic Starch+50 mM Na-Acetate (pH 5.2)
4. 25 mg/mL Dual Cationic-PO4 Starch+50 mM Na-Acetate (pH 5.2)
5. 12.5 mg/mL Dual Cationic-PO4 Starch+50 mM Na-Acetate (pH 5.2)
6. 50 mg/mL Dual PO4-Cationic Inositol+50 mM Na-Acetate (pH 5.2)
7. 25 mg/mL Dual PO4-Cationic Inositol+50 mM Na-Acetate (pH 5.2)
8. 50 mg/mL Dual Cationic-PO4 Inositol+50 mM Na-Acetate (pH 5.2)
9. 25 mg/mL Dual Cationic-PO4 Inositol+50 mM Na-Acetate (pH 5.2)
10. −80° C.

Following dilution of each sample in each respective RNA storage environment, samples were stored in a thermal cycler at 60° C. for about 24 hours and then analyzed by denaturing agarose gel electrophoresis as shown in FIGS. 40 C and D.

### Example 84

### Accelerated RNA Stability Testing at 60° C.

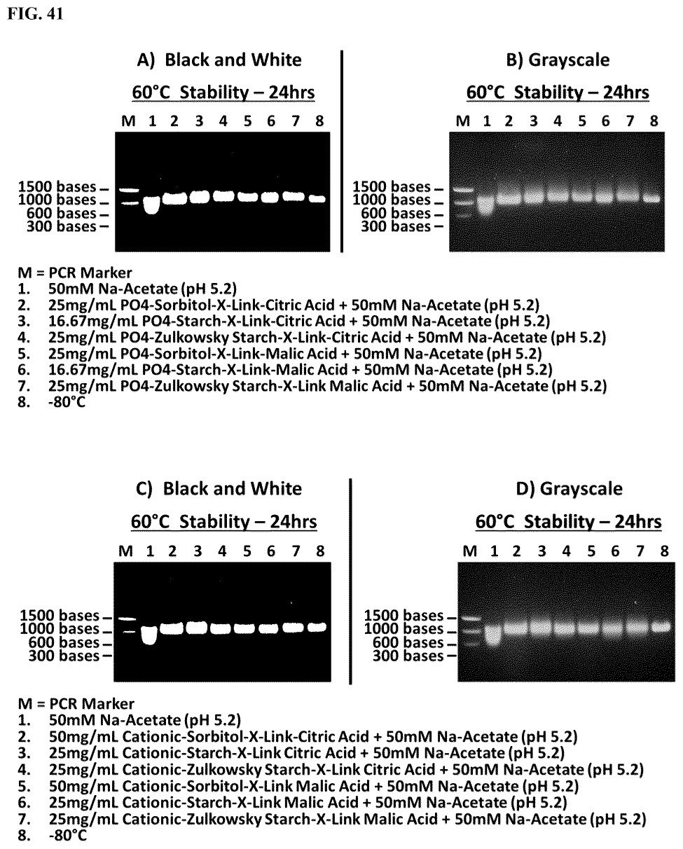

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 41A and B.
M=PCR Marker
1. 50 mM Na-Acetate (pH 5.2)
2. 25 mg/mL PO4-Sorbitol-X-Link-Citric Acid+50 mM Na-Acetate (pH 5.2)
3. 16.67 mg/mL PO4-Starch-X-Link-Citric Acid+50 mM Na-Acetate (pH 5.2)
4. 25 mg/mL PO4-Zulkowsky Starch-X-Link-Citric Acid+50 mM Na-Acetate (pH 5.2)
5. 25 mg/mL PO4-Sorbitol-X-Link-Malic Acid+50 mM Na-Acetate (pH 5.2)
6. 16.67 mg/mL PO4-Starch-X-Link-Malic Acid+50 mM Na-Acetate (pH 5.2)
7. 25 mg/mL PO4-Zulkowsky Starch-X-Link Malic Acid+50 mM Na-Acetate (pH 5.2)
8. −80° C.

Following dilution of each sample in each respective RNA storage environment, samples were stored in a thermal cycler at 60° C. for about 24 hours and then analyzed by denaturing agarose gel electrophoresis as shown in FIGS. 41A and B.

### Example 85

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 41 C and D.
M=PCR Marker
1. 50 mM Na-Acetate (pH 5.2)
2. 50 mg/mL Cationic-Sorbitol-X-Link-Citric Acid+50 mM Na-Acetate (pH 5.2)
3. 25 mg/mL Cationic-Starch-X-Link Citric Acid+50 mM Na-Acetate (pH 5.2)
4. 25 mg/mL Cationic-Zulkowsky Starch-X-Link Citric Acid+50 mM Na-Acetate (pH 5.2)
5. 50 mg/mL Cationic-Sorbitol-X-Link Malic Acid+50 mM Na-Acetate (pH 5.2)
6. 25 mg/mL Cationic-Starch-X-Link Malic Acid+50 mM Na-Acetate (pH 5.2)
7. 25 mg/mL Cationic-Zulkowsky Starch-X-Link Malic Acid+50 mM Na-Acetate (pH 5.2)
8. −80° C.

Following dilution of each sample in each respective RNA storage environment, samples were stored in a thermal cycler at 60° C. for about 24 hours and then analyzed by denaturing agarose gel electrophoresis as shown in FIGS. 41 C and D.

### Example 86

### Accelerated RNA Stability Testing at 60° C.

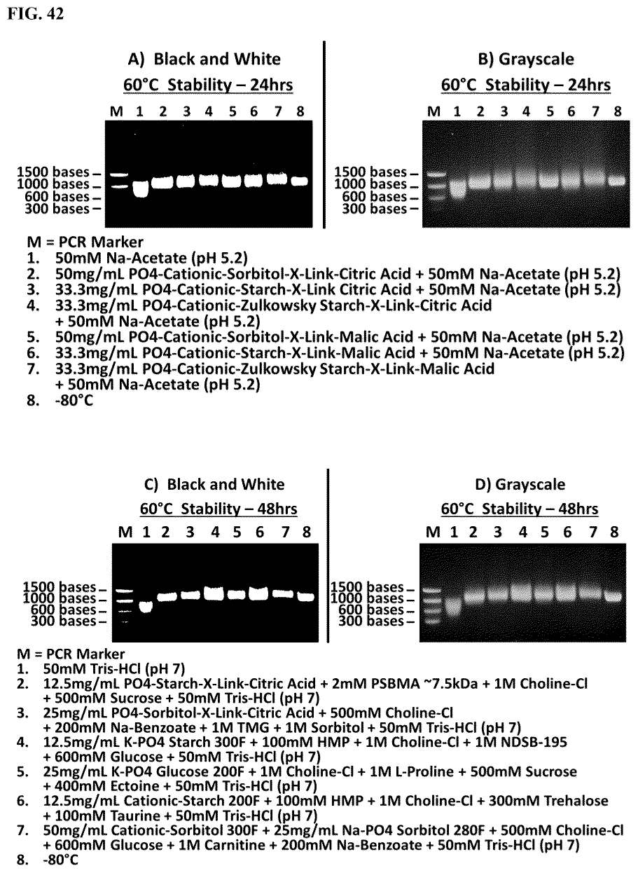

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 42A and B.
M=PCR Marker
1. 50 mM Na-Acetate (pH 5.2)
2. 50 mg/mL PO4-Cationic-Sorbitol-X-Link-Citric Acid+50 mM Na-Acetate (pH 5.2)
3. 33.3 mg/mL PO4-Cationic-Starch-X-Link Citric Acid+50 mM Na-Acetate (pH 5.2)
4. 33.3 mg/mL PO4-Cationic-Zulkowsky Starch-X-Link-Citric Acid+50 mM Na-Acetate (pH 5.2)
5. 50 mg/mL PO4-Cationic-Sorbitol-X-Link-Malic Acid+50 mM Na-Acetate (pH 5.2)
6. 33.3 mg/mL PO4-Cationic-Starch-X-Link-Malic Acid+50 mM Na-Acetate (pH 5.2)
7. 33.3 mg/mL PO4-Cationic-Zulkowsky Starch-X-Link-Malic Acid+50 mM Na-Acetate (pH 5.2)
8. −80° C.

Following dilution of each sample in each respective RNA storage environment, samples were stored in a thermal cycler at 60° C. for about 24 hours and then analyzed by denaturing agarose gel electrophoresis as shown in FIGS. 42A and B.

### Example 87

### Accelerated RNA Stability Testing at 60° C.

In vitro transcribed RNA was diluted at a ratio of about 1:10 (about 100-300 μg/mL) in different compositions containing either 50 mM Tris-HCl (pH 7), or one or more compounds in compositions with 50 mM Tris-HCl (pH 7) as shown in FIGS. 42 C and D.
M=PCR Marker
1. 50 mM Tris-HCl (pH 7)
2. 12.5 mg/mL PO4-Starch-X-Link-Citric Acid+2 mM PSBMA ˜7.5 kDa+1M Choline-Cl+500 mM Sucrose+50 mM Tris-HCl (pH 7)
3. 25 mg/mL PO4-Sorbitol-X-Link-Citric Acid+500 mM Choline-Cl+200 mM Na-Benzoate+1M TMG+1M Sorbitol+50 mM Tris-HCl (pH 7)
4. 12.5 mg/mL K—PO4 Starch 300F+100 mM HMP+1M Choline-Cl+1M NDSB-195+600 mM Glucose+50 mM Tris-HCl (pH 7)
5. 25 mg/mL K—PO4 Glucose 200F+1M Choline-Cl+1M L-Proline+500 mM Sucrose+400 mM Ectoine+50 mM Tris-HCl (pH 7)
6. 12.5 mg/mL Cationic-Starch 200F+100 mM HMP+1M Choline-Cl+300 mM Trehalose+100 mM Taurine+50 mM Tris-HCl (pH 7)
7. 50 mg/mL Cationic-Sorbitol 300F+25 mg/mL Na—PO4 Sorbitol 280F+500 mM Choline-Cl+600 mM Glucose+1M L-Carnitine+200 mM Na-Benzoate+50 mM Tris-HCl (pH 7)
8. −80° C.

Following dilution of each sample in each respective RNA storage environment, samples were stored in a thermal cycler at 60° C. for about 48 hours and then analyzed by denaturing agarose gel electrophoresis as shown in FIGS. 42 C and D.

### Example 88

### Accelerated RNA Stability Testing at 60° C.

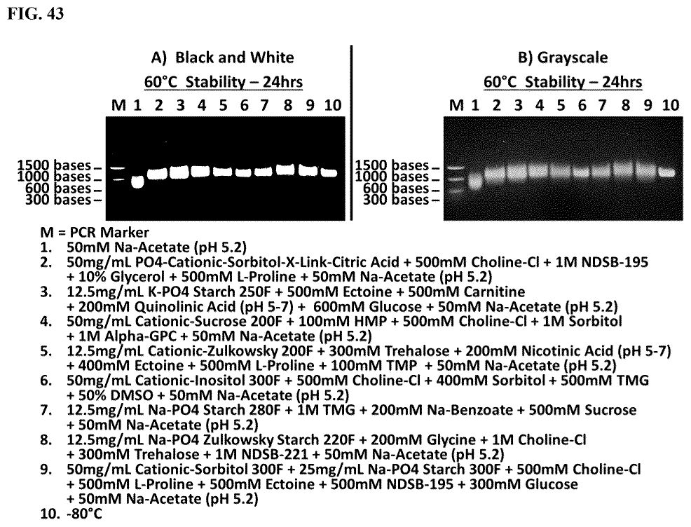

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 43A and B.
M=PCR Marker
1. 50 mM Na-Acetate (pH 5.2)
2. 50 mg/mL PO4-Cationic-Sorbitol-X-Link-Citric Acid+500 mM Choline-Cl+1M NDSB-195+10% Glycerol+500 mM L-Proline+50 mM Na-Acetate (pH 5.2)
3. 12.5 mg/mL K—PO4 Starch 250F+500 mM Ectoine+500 mM L-Carnitine+200 mM Quinolinic Acid (pH 5-7)+600 mM Glucose+50 mM Na-Acetate (pH 5.2)
4. 50 mg/mL Cationic-Sucrose 200F+100 mM HMP+500 mM Choline-Cl+1M Sorbitol+1M Alpha-GPC+50 mM Na-Acetate (pH 5.2)
5. 12.5 mg/mL Cationic-Zulkowsky 200F+300 mM Trehalose+200 mM Nicotinic Acid (pH 5-7)+400 mM Ectoine+500 mM L-Proline+100 mM TMP+50 mM Na-Acetate (pH 5.2)
6. 50 mg/mL Cationic-Inositol 300F+500 mM Choline-Cl+400 mM Sorbitol+500 mM TMG+50% DMSO+50 mM Na-Acetate (pH 5.2)
7. 12.5 mg/mL Na—PO4 Starch 280F+1M TMG+200 mM Na-Benzoate+500 mM Sucrose+50 mM Na-Acetate (pH 5.2)
8. 12.5 mg/mL Na—PO4 Zulkowsky Starch 220F+200 mM Glycine+1M Choline-Cl+300 mM Trehalose+1M NDSB-221+50 mM Na-Acetate (pH 5.2)
9. 50 mg/mL Cationic-Sorbitol 300F+25 mg/mL Na—PO4 Starch 300F+500 mM Choline-Cl+500 mM L-Proline+500 mM Ectoine+500 mM NDSB-195+300 mM Glucose+50 mM Na-Acetate (pH 5.2)
10. −80° C.

Following dilution of each sample in each respective RNA storage environment, samples were stored in a thermal cycler at 60° C. for about 24 hours and then analyzed by denaturing agarose gel electrophoresis as shown in FIGS. 43A and B.

### Example 89

### Accelerated RNA Stability Testing at 60° C.

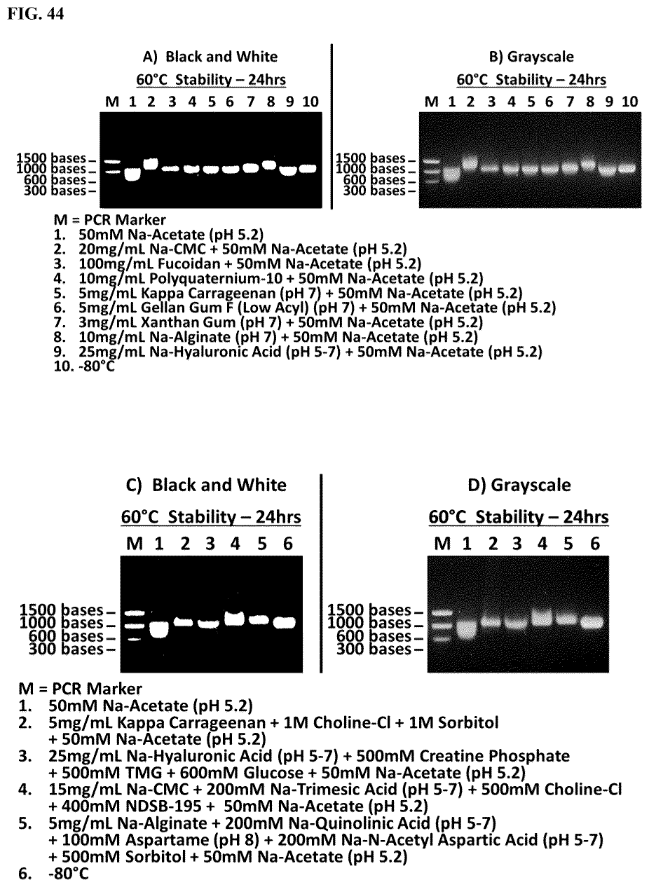

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 44A and B.
M=PCR Marker
1. 50 mM Na-Acetate (pH 5.2)
2. 20 mg/mL Na-CMC+50 mM Na-Acetate (pH 5.2)
3. 100 mg/mL Fucoidan+50 mM Na-Acetate (pH 5.2)
4. 10 mg/mL Polyquaternium-10+50 mM Na-Acetate (pH 5.2)
5. 5 mg/mL Kappa Carrageenan (pH 7)+50 mM Na-Acetate (pH 5.2)
6. 5 mg/mL Gellan Gum F (Low Acyl) (pH 7)+50 mM Na-Acetate (pH 5.2)
7. 3 mg/mL Xanthan Gum (pH 7)+50 mM Na-Acetate (pH 5.2)
8. 10 mg/mL Na-Alginate (pH 7)+50 mM Na-Acetate (pH 5.2)
9. 25 mg/mL Na-Hyaluronic Acid (pH 5-7)+50 mM Na-Acetate (pH 5.2)
10. −80° C.

Following dilution of each sample in each respective RNA storage environment, samples were stored in a thermal cycler at 60° C. for about 24 hours and then analyzed by denaturing agarose gel electrophoresis as shown in FIGS. 44A and B.

Prior to analysis by agarose gel electrophoresis, the sample with Polyquaternium-10 was incubated with 2 mM PAA for 1 hr at room temperature to help improve analysis by gel electrophoresis. Prior to analysis by agarose gel electrophoresis, all samples were diluted between 2-3× with water to reduce viscosity and help improve analysis by gel electrophoresis.

The following were purchased from: Na-Carboxymethylcellulose (CMC) ˜90 kDa (Sigma-Aldrich, Product #419273), Fucoidan (Cayman Chemical, Product #20357), Polyquaternium-10 (Divinity Cosmetic Supply, Port St Lucie, FL), Kappa Carrageenan (Modernist Pantry, Product #1011-50), Gellan Gum F-Low Acyl(Modernist Pantry, Product #1028), Xanthan Gum (Modernist Pantry, Product #1019-50), Na-Alginate (Modernist Pantry, Product #1007-50), Hyaluronic Acid ˜10 kDa (Pure Health Botanicals, LLC, Saint Charles, IL, dba as ebay purehealthsolutions)

### Example 90

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 44 C and D.
M=PCR Marker
1. 50 mM Na-Acetate (pH 5.2)
2. 5 mg/mL Kappa Carrageenan+1M Choline-Cl+1M Sorbitol+50 mM Na-Acetate (pH 5.2)
3. 25 mg/mL Na-Hyaluronic Acid (pH 5-7)+500 mM Creatine Phosphate+500 mM TMG+600 mM Glucose+50 mM Na-Acetate (pH 5.2)
4. 15 mg/mL Na-CMC+200 mM Na-Trimesic Acid (pH 5-7)+500 mM Choline-Cl+400 mM NDSB-195+50 mM Na-Acetate (pH 5.2)
5. 5 mg/mL Na-Alginate+200 mM Na-Quinolinic Acid (pH 5-7)+100 mM Aspartame (pH 8)+200 mM Na—N-Acetyl Aspartic Acid (pH 5-7)+500 mM Sorbitol+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 44 C and D.

Prior to analysis by agarose gel electrophoresis, all samples were diluted between 2-3× with water to reduce viscosity and help improve analysis by gel electrophoresis.

### Example 91

### Accelerated RNA Stability Testing at 60° C.

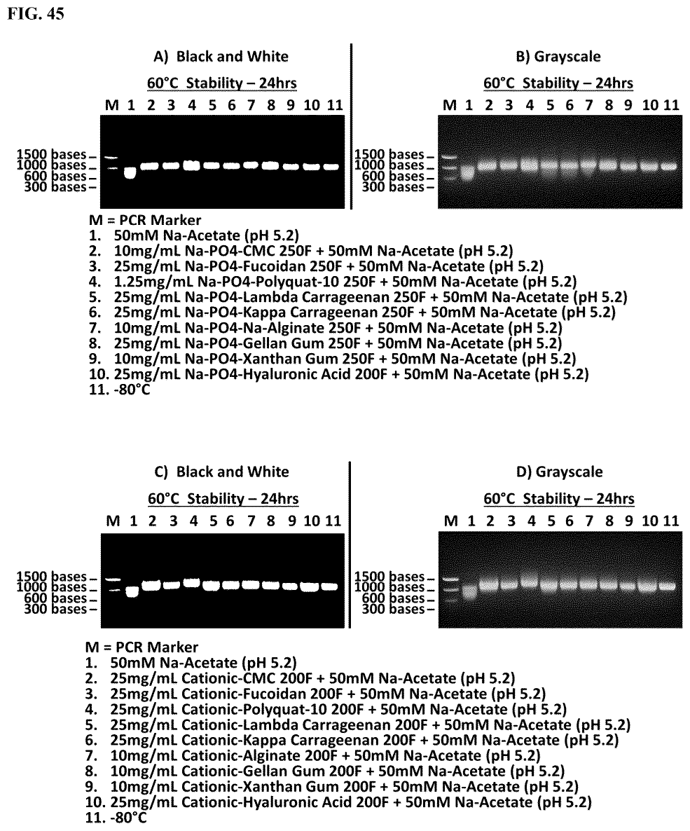

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 45A and B.
M=PCR Marker
1. 50 mM Na-Acetate (pH 5.2)
2. 10 mg/mL Na—PO4-CMC 250F+50 mM Na-Acetate (pH 5.2)
3. 25 mg/mL Na—PO4-Fucoidan 250F+50 mM Na-Acetate (pH 5.2)
4. 1.25 mg/mL Na—PO4-Polyquat-10 250F+50 mM Na-Acetate (pH 5.2)
5. 25 mg/mL Na—PO4-Lambda Carrageenan 250F+50 mM Na-Acetate (pH 5.2)
6. 25 mg/mL Na—PO4-Kappa Carrageenan 250F+50 mM Na-Acetate (pH 5.2)
7. 10 mg/mL Na—PO4-Na-Alginate 250F+50 mM Na-Acetate (pH 5.2)
8. 25 mg/mL Na—PO4-Gellan Gum 250F+50 mM Na-Acetate (pH 5.2)
9. 10 mg/mL Na—PO4-Xanthan Gum 250F+50 mM Na-Acetate (pH 5.2)
10. 25 mg/mL Na—PO4-Hyaluronic Acid 200F+50 mM Na-Acetate (pH 5.2)
11. −80° C.

Following dilution of each sample in each respective RNA storage environment, samples were stored in a thermal cycler at 60° C. for about 24 hours and then analyzed by denaturing agarose gel electrophoresis as shown in FIGS. 45A and B.

Prior to analysis by agarose gel electrophoresis, the sample with Na—PO4-Polyquat-10 250F was incubated with 2 mM PAA ˜8 kDa for 1 hr at room temperature to help improve analysis by gel electrophoresis.

### Example 92

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 45 C and D.
M=PCR Marker
1. 50 mM Na-Acetate (pH 5.2)
2. 25 mg/mL Cationic-CMC 200F+50 mM Na-Acetate (pH 5.2)
3. 25 mg/mL Cationic-Fucoidan 200F+50 mM Na-Acetate (pH 5.2)
4. 25 mg/mL Cationic-Polyquat-10 200F+50 mM Na-Acetate (pH 5.2)
5. 25 mg/mL Cationic-Lambda Carrageenan 200F+50 mM Na-Acetate (pH 5.2)
6. 25 mg/mL Cationic-Kappa Carrageenan 200F+50 mM Na-Acetate (pH 5.2)
7. 10 mg/mL Cationic-Alginate 200F+50 mM Na-Acetate (pH 5.2)
8. 10 mg/mL Cationic-Gellan Gum 200F+50 mM Na-Acetate (pH 5.2)
9. 10 mg/mL Cationic-Xanthan Gum 200F+50 mM Na-Acetate (pH 5.2)
10. 25 mg/mL Cationic-Hyaluronic Acid 200F+50 mM Na-Acetate (pH 5.2)
11. −80° C.

Following dilution of each sample in each respective RNA storage environment, samples were stored in a thermal cycler at 60° C. for about 24 hours and then analyzed by denaturing agarose gel electrophoresis as shown in FIGS. 45 C and D.

Prior to analysis by agarose gel electrophoresis, the sample with Cationic-Polyquat-10 200F was incubated with 1 mM PAA ˜8 kDa for 1 hr at room temperature to help improve analysis by gel electrophoresis.

### Example 93

### Accelerated RNA Stability Testing at 60° C.

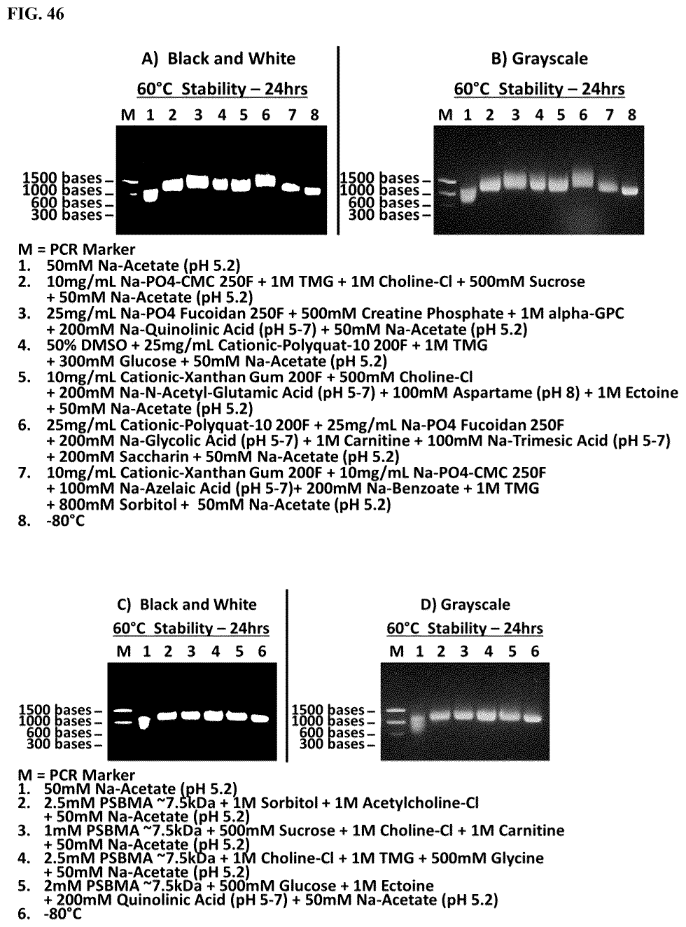

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 46A and B.
M=PCR Marker
1. 50 mM Na-Acetate (pH 5.2)
2. 10 mg/mL Na—PO4-CMC 250F+1M TMG+1M Choline-Cl+500 mM Sucrose+50 mM Na-Acetate (pH 5.2)
3. 25 mg/mL Na—PO4 Fucoidan 250F+500 mM Creatine Phosphate+1M alpha-GPC+200 mM Na-Quinolinic Acid (pH 5-7)+50 mM Na-Acetate (pH 5.2)
4. 50% DMSO+25 mg/mL Cationic-Polyquat-10 200F+1M TMG+300 mM Glucose+50 mM Na-Acetate (pH 5.2)
5. 10 mg/mL Cationic-Xanthan Gum 200F+500 mM Choline-Cl+200 mM Na—N-Acetyl-Glutamic Acid (pH 5-7)+100 mM Aspartame (pH 8)+1M Ectoine+50 mM Na-Acetate (pH 5.2)
6. 25 mg/mL Cationic-Polyquat-10 200F+25 mg/mL Na—PO4 Fucoidan 250F+200 mM Na-Glycolic Acid (pH 5-7)+1M L-Carnitine+100 mM Na-Trimesic Acid (pH 5-7)+200 mM Saccharin+50 mM Na-Acetate (pH 5.2)
7. 10 mg/mL Cationic-Xanthan Gum 200F+10 mg/mL Na—PO4-CMC 250F+100 mM Na-Azelaic Acid (pH 5-7)+200 mM Na-Benzoate+1M TMG+800 mM Sorbitol+50 mM Na-Acetate (pH 5.2)
8. −80° C.

Following dilution of each sample in each respective RNA storage environment, samples were stored in a thermal cycler at 60° C. for about 24 hours and then analyzed by denaturing agarose gel electrophoresis as shown in FIGS. 46A and B.

Prior to analysis by agarose gel electrophoresis, the sample with Cationic-Polyquat-10 200F was incubated with 1 mM PAA ˜8 kDa for 1 hr at room temperature to help improve analysis by gel electrophoresis.

### Example 94

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 46 C and D.
M=PCR Marker
1. 50 mM Na-Acetate (pH 5.2)
2. 2.5 mM PSBMA ˜7.5 kDa+1M Sorbitol+1M Acetylcholine-Cl+50 mM Na-Acetate (pH 5.2)
3. 1 mM PSBMA ˜7.5 kDa+500 mM Sucrose+1M Choline-Cl+1M L-Carnitine+50 mM Na-Acetate (pH 5.2)
4. 2.5 mM PSBMA ˜7.5 kDa+1M Choline-Cl+1M TMG+500 mM Glycine+50 mM Na-Acetate (pH 5.2)
5. 2 mM PSBMA ˜7.5 kDa+500 mM Glucose+1M Ectoine+200 mM Quinolinic Acid (pH 5-7)+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 46 C and D.

### Example 95

### Accelerated RNA Stability Testing at 60° C.

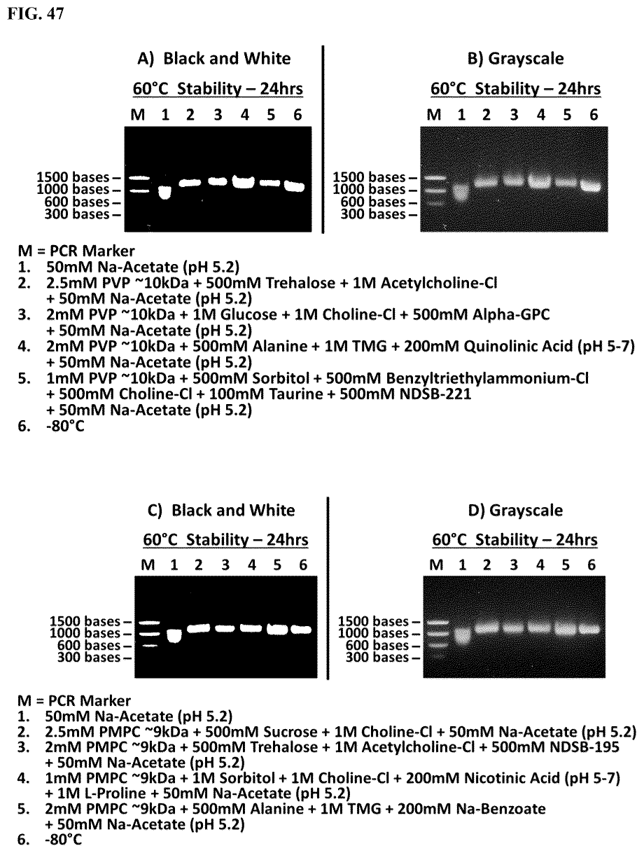

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 47A and B.
M=PCR Marker
1. 50 mM Na-Acetate (pH 5.2)
2. 2.5 mM PVP ˜10 kDa+500 mM Trehalose+1M Acetylcholine-Cl+50 mM Na-Acetate (pH 5.2)
3. 2 mM PVP ˜10 kDa+1M Glucose+1M Choline-Cl+500 mM Alpha-GPC+50 mM Na-Acetate (pH 5.2)
4. 2 mM PVP ˜10 kDa+500 mM Alanine+1M TMG+200 mM Quinolinic Acid (pH 5-7)+50 mM Na-Acetate (pH 5.2)
5. 1 mM PVP ˜10 kDa+500 mM Sorbitol+500 mM Benzyltriethylammonium-Cl+500 mM Choline-Cl+100 mM Taurine+500 mM NDSB-221+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 47A and B.

Prior to analysis by agarose gel electrophoresis, the sample with benzyltriethylammonium was incubated with 1 mM PAA ˜8 kDa for 1 hr at room temperature to help improve analysis by gel electrophoresis.

### Example 96

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 47 C and D.
M=PCR Marker
1. 50 mM Na-Acetate (pH 5.2)
2. 2.5 mM PMPC ˜9 kDa+500 mM Sucrose+1M Choline-Cl+50 mM Na-Acetate (pH 5.2)
3. 2 mM PMPC ˜9 kDa+500 mM Trehalose+1M Acetylcholine-Cl+500 mM NDSB-195+50 mM Na-Acetate (pH 5.2)
4. 1 mM PMPC ˜9 kDa+1M Sorbitol+1M Choline-Cl+200 mM Nicotinic Acid (pH 5-7)+1M L-Proline+50 mM Na-Acetate (pH 5.2)
5. 2 mM PMPC ˜9 kDa+500 mM Alanine+1M TMG+200 mM Na-Benzoate+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 47 C and D.

### Example 97

### Accelerated RNA Stability Testing at 60° C.

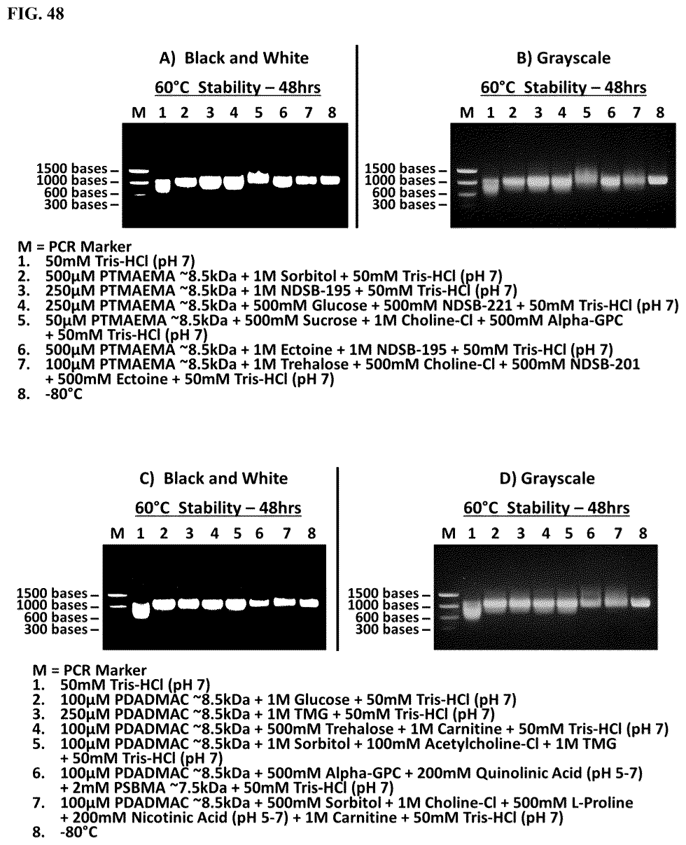

In vitro transcribed RNA was diluted at a ratio of about 1:10 (about 100-300 μg/mL) in different compositions containing either 50 mM Tris-HCl (pH 7), or one or more compounds in compositions with 50 mM Tris-HCl (pH 7) as shown in FIGS. 48A and B.
M=PCR Marker
1. 50 mM Tris-HCl (pH 7)
2. 500 UM PTMAEMA ˜8.5 kDa+1M Sorbitol+50 mM Tris-HCl (pH 7)
3. 250 UM PTMAEMA ˜8.5 kDa+1M NDSB-195+50 mM Tris-HCl (pH 7)
4. 250 UM PTMAEMA ˜8.5 kDa+500 mM Glucose+500 mM NDSB-221+50 mM Tris-HCl (pH 7)
5. 50 UM PTMAEMA ˜8.5 kDa+500 mM Sucrose+1M Choline-Cl+500 mM Alpha-GPC+50 mM Tris-HCl (pH 7)
6. 500 UM PTMAEMA ˜8.5 kDa+1M Ectoine+1M NDSB-195+50 mM Tris-HCl (pH 7)
7. 100 UM PTMAEMA ˜8.5 kDa+1M Trehalose+500 mM Choline-Cl+500 mM NDSB-+500 mM Ectoine+50 mM Tris-HCl (pH 7)
8. −80° C.

Following dilution of each sample in each respective RNA storage environment, samples were stored in a thermal cycler at 60° C. for about 48 hours and then analyzed by denaturing agarose gel electrophoresis as shown in FIGS. 48A and B.

Prior to analysis by agarose gel electrophoresis, all PTMAEMA samples were incubated with 2 mM PAA ˜8 kDa for 30 min at 70° C. to help improve analysis by gel electrophoresis.

### Example 98

### Accelerated RNA Stability Testing at 60° C.

In vitro transcribed RNA was diluted at a ratio of about 1:10 (about 100-300 μg/mL) in different compositions containing either 50 mM Tris-HCl (pH 7), or one or more compounds in compositions with 50 mM Tris-HCl (pH 7) as shown in FIGS. 48 C and D.
M=PCR Marker
1. 50 mM Tris-HCl (pH 7)
2. 100 μM PDADMAC ˜8.5 kDa+1M Glucose+50 mM Tris-HCl (pH 7)
3. 250 μM PDADMAC ˜8.5 kDa+1M TMG+50 mM Tris-HCl (pH 7)
4. 100 μM PDADMAC ˜8.5 kDa+500 mM Trehalose+1M L-Carnitine+50 mM Tris-HCl (pH 7)
5. 100 μM PDADMAC ˜8.5 kDa+1M Sorbitol+100 mM Acetylcholine-Cl+1M TMG+50 mM Tris-HCl (pH 7)
6. 100 μM PDADMAC ˜8.5 kDa+500 mM Alpha-GPC+200 mM Quinolinic Acid (pH 5-7)+2 mM PSBMA ˜7.5 kDa+50 mM Tris-HCl (pH 7)
7. 100 μM PDADMAC ˜8.5 kDa+500 mM Sorbitol+1M Choline-Cl+500 mM L-Proline+200 mM Nicotinic Acid (pH 5-7)+1M L-Carnitine+50 mM Tris-HCl (pH 7)
8. −80° C.

Following dilution of each sample in each respective RNA storage environment, samples were stored in a thermal cycler at 60° C. for about 48 hours and then analyzed by denaturing agarose gel electrophoresis as shown in FIGS. 48 C and D.

Prior to analysis by agarose gel electrophoresis, all PDADMAC samples were incubated with 2 mM PAA ˜8 kDa for 1 hr at room temperature to help improve analysis by agarose gel electrophoresis.

### Example 99

### Accelerated RNA Stability Testing at 60° C.

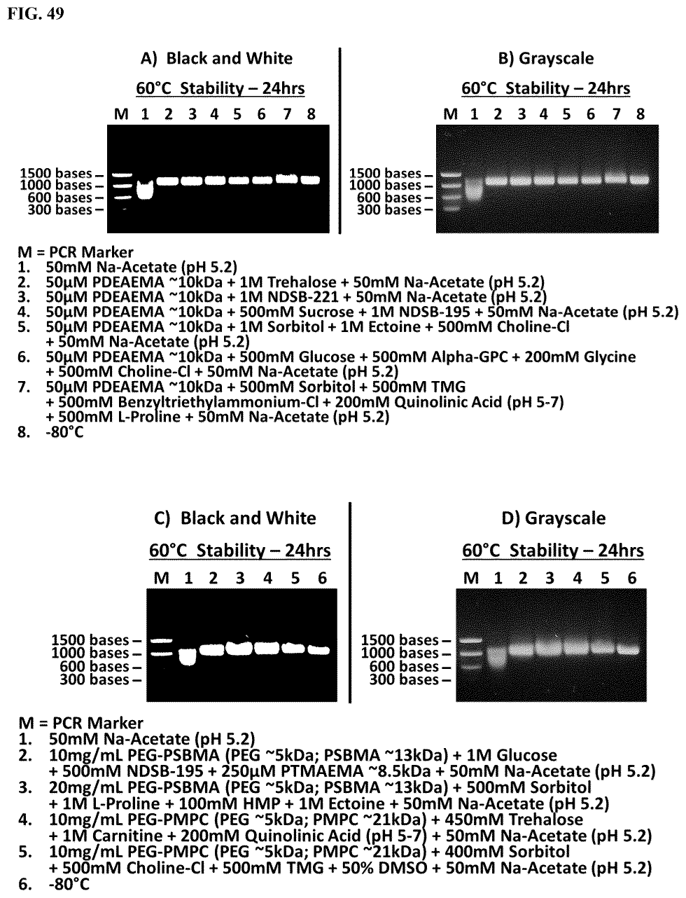

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 49A and B.
M=PCR Marker
1. 50 mM Na-Acetate (pH 5.2)
2. 50 μM PDEAEMA ˜10 kDa+1M Trehalose+50 mM Na-Acetate (pH 5.2)
3. 50 μM PDEAEMA ˜10 kDa+1M NDSB-221+50 mM Na-Acetate (pH 5.2)
4. 50 μM PDEAEMA ˜10 kDa+500 mM Sucrose+1M NDSB-195+50 mM Na-Acetate (pH 5.2)
5. 50 μM PDEAEMA ˜10 kDa+1M Sorbitol+1M Ectoine+500 mM Choline-Cl+50 mM Na-Acetate (pH 5.2)
6. 50 μM PDEAEMA ˜10 kDa+500 mM Glucose+500 mM Alpha-GPC+200 mM Glycine+500 mM Choline-Cl+50 mM Na-Acetate (pH 5.2)
7. 50 μM PDEAEMA ˜10 kDa+500 mM Sorbitol+500 mM TMG+500 mM Benzyltriethylammonium-Cl+200 mM Quinolinic Acid (pH 5-7)+500 mM L-Proline+50 mM Na-Acetate (pH 5.2)
8. −80° C.

Following dilution of each sample in each respective RNA storage environment, samples were stored in a thermal cycler at 60° C. for about 24 hours and then analyzed by denaturing agarose gel electrophoresis as shown in FIGS. 49A and B.

Prior to analysis by agarose gel electrophoresis, all PDEAEMA samples were incubated with 1 mM PAA ˜8 kDa for 1 hr at room temperature to help improve analysis by gel electrophoresis.

### Example 100

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 49 C and D.
M=PCR Marker
1. 50 mM Na-Acetate (pH 5.2)
2. 10 mg/mL PEG-PSBMA (PEG ˜5 kDa; PSBMA ˜13 kDa)+1M Glucose+500 mM NDSB-195+250 μM PTMAEMA ˜8.5 kDa+50 mM Na-Acetate (pH 5.2)
3. 20 mg/mL PEG-PSBMA (PEG ˜5 kDa; PSBMA ˜13 kDa)+500 mM Sorbitol+1M L-Proline+100 mM HMP+1M Ectoine+50 mM Na-Acetate (pH 5.2)
4. 10 mg/mL PEG-PMPC (PEG ˜5 kDa; PMPC ˜21 kDa)+450 mM Trehalose+1M L-Carnitine+200 mM Quinolinic Acid (pH 5-7)+50 mM Na-Acetate (pH 5.2)
5. 10 mg/mL PEG-PMPC (PEG ˜5 kDa; PMPC ˜21 kDa)+400 mM Sorbitol+500 mM Choline-Cl+500 mM TMG+50% DMSO+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 49 C and D.

Prior to analysis by agarose gel electrophoresis, the sample with PTMAEMA was incubated with 1 mM PAA ˜8 kDa for 30 min at 70° C. to help improve analysis by gel electrophoresis.

### Example 101

### Accelerated RNA Stability Testing at 60° C.

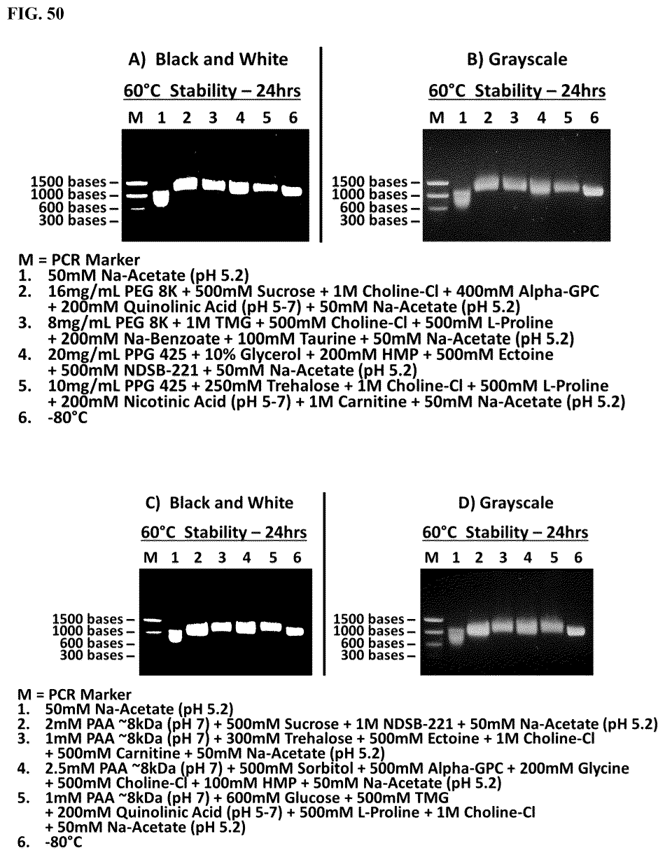

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 50A and B.
M=PCR Marker
1. 50 mM Na-Acetate (pH 5.2)
2. 16 mg/mL PEG 8K+500 mM Sucrose+1M Choline-Cl+400 mM Alpha-GPC+200 mM Quinolinic Acid (pH 5-7)+50 mM Na-Acetate (pH 5.2)
3. 8 mg/mL PEG 8K+1M TMG+500 mM Choline-Cl+500 mM L-Proline+200 mM Na-Benzoate+100 mM Taurine+50 mM Na-Acetate (pH 5.2)
4. 20 mg/mL PPG 425+10% Glycerol+200 mM HMP+500 mM Ectoine+500 mM NDSB-221+50 mM Na-Acetate (pH 5.2)
5. 10 mg/mL PPG 425+250 mM Trehalose+1M Choline-Cl+500 mM L-Proline+200 mM Nicotinic Acid (pH 5-7)+1M L-Carnitine+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 50A and B.

The following were purchased from: polyethylene glycol (˜8 kDa) (PEG 8K) (Sigma-Aldrich, Product #89510), polypropylene glycol (Mn 425) (PPG 425) (Sigma-Aldrich, Product #202304)

### Example 102

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 50 C and D.
M=PCR Marker
1. 50 mM Na-Acetate (pH 5.2)
2. 2 mM PAA ˜8 kDa (pH 7)+500 mM Sucrose+1M NDSB-221+50 mM Na-Acetate (pH 5.2)
3. 1 mM PAA ˜8 kDa (pH 7)+300 mM Trehalose+500 mM Ectoine+1M Choline-Cl+500 mM L-Carnitine+50 mM Na-Acetate (pH 5.2)
4. 2.5 mM PAA ˜8 kDa (pH 7)+500 mM Sorbitol+500 mM Alpha-GPC+200 mM Glycine+500 mM Choline-Cl+100 mM HMP+50 mM Na-Acetate (pH 5.2)
5. 1 mM PAA ˜8 kDa (pH 7)+600 mM Glucose+500 mM TMG+200 mM Quinolinic Acid (pH 5-7)+500 mM L-Proline+1M Choline-Cl+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 50 C and D.

### Example 103

### Accelerated RNA Stability Testing at 60° C.

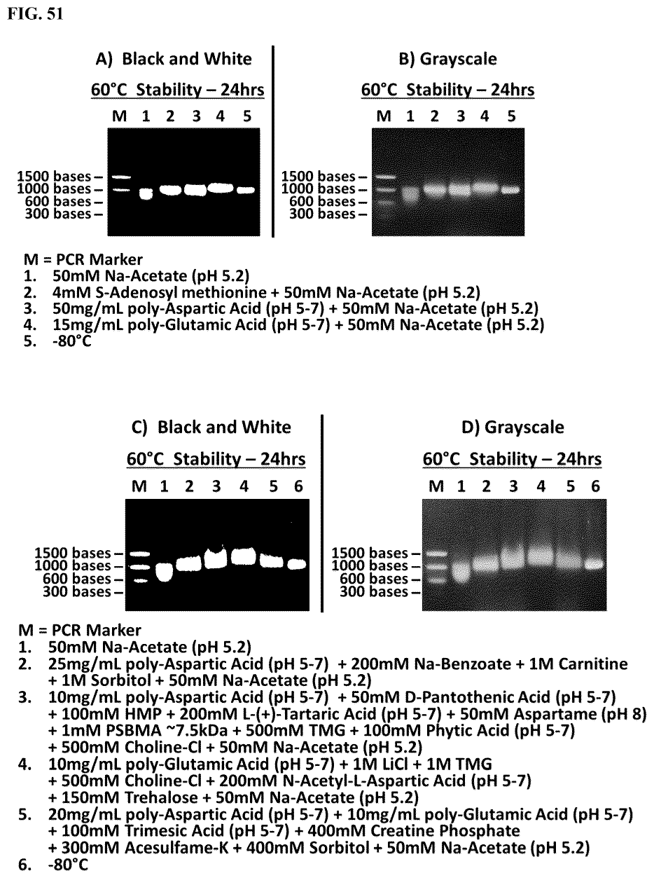

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 51A and B.
M=PCR Marker
1. 50 mM Na-Acetate (pH 5.2)
2. 4 mM S-Adenosyl methionine+50 mM Na-Acetate (pH 5.2)
3. 50 mg/mL poly-Aspartic Acid (pH 5-7)+50 mM Na-Acetate (pH 5.2)
4. 15 mg/mL poly-Glutamic Acid (pH 5-7)+50 mM Na-Acetate (pH 5.2) 5. −80° C.

Following dilution of each sample in each respective RNA storage environment, samples were stored in a thermal cycler at 60° C. for about 24 hours and then analyzed by denaturing agarose gel electrophoresis as shown in FIGS. 51A and B.

The following were purchased from: poly-Glutamic Acid (Pure Health Botanicals, LLC, Saint Charles, IL, dba as ebay purehealthsolutions), S-Adenosyl Methionine (Pure Bulk).

### Example 104

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 51 C and D.
M=PCR Marker
1. 50 mM Na-Acetate (pH 5.2)
2. 25 mg/mL poly-Aspartic Acid (pH 5-7)+200 mM Na-Benzoate+1M L-Carnitine+1M Sorbitol+50 mM Na-Acetate (pH 5.2)
3. 10 mg/mL poly-Aspartic Acid (pH 5-7)+50 mM D-Pantothenic Acid (pH 5-7)+100 mM HMP+200 mM L-(+)-Tartaric Acid (pH 5-7)+50 mM Aspartame (pH 8)+1 mM PSBMA ˜7.5 kDa+500 mM TMG+100 mM Phytic Acid (pH 5-7)+500 mM Choline-Cl+50 mM Na-Acetate (pH 5.2)
4. 10 mg/mL poly-Glutamic Acid (pH 5-7)+1M LiCl+1M TMG+500 mM Choline-Cl+200 mM N-Acetyl-L-Aspartic Acid (pH 5-7)+150 mM Trehalose+50 mM Na-Acetate (pH 5.2)
5. 20 mg/mL poly-Aspartic Acid (pH 5-7)+10 mg/mL poly-Glutamic Acid (pH 5-7)+100 mM Trimesic Acid (pH 5-7)+400 mM Creatine Phosphate+300 mM Acesulfame-K+400 mM Sorbitol+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 51 C and D.

### Example 105

### Accelerated RNA Stability Testing at 60° C.

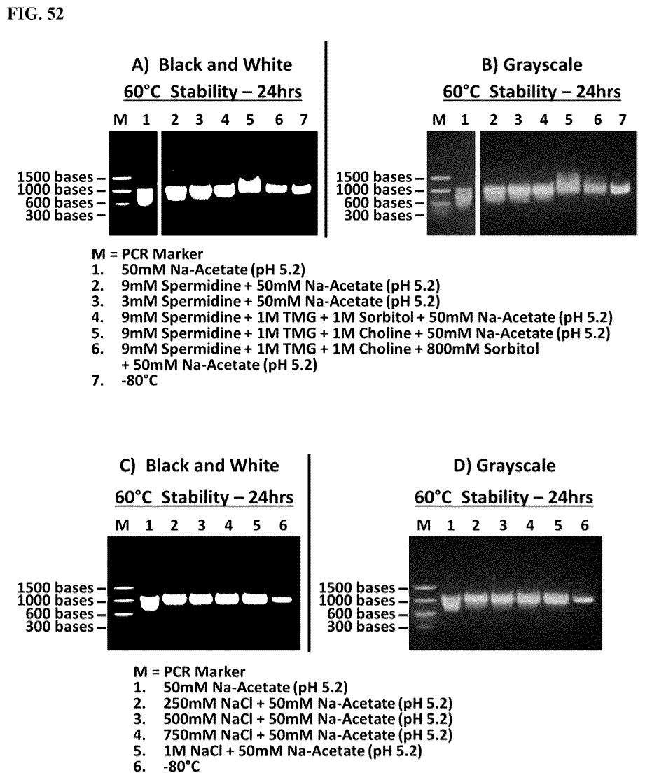

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 52A and B.
M=PCR Marker
1. 50 mM Na-Acetate (pH 5.2)
2. 9 mM Spermidine+50 mM Na-Acetate (pH 5.2)
3. 3 mM Spermidine+50 mM Na-Acetate (pH 5.2)
4. 9 mM Spermidine+1M TMG+1M Sorbitol+50 mM Na-Acetate (pH 5.2)
5. 9 mM Spermidine+1M TMG+1M Choline-Cl+50 mM Na-Acetate (pH 5.2)
6. 9 mM Spermidine+1M TMG+1M Choline-Cl+800 mM Sorbitol+50 mM Na-Acetate (pH 5.2)
7. −80° C.

Following dilution of each sample in each respective RNA storage environment, samples were stored in a thermal cycler at 60° C. for about 24 hours and then analyzed by denaturing agarose gel electrophoresis as shown in FIGS. 52A and B.

Prior to analysis by agarose gel electrophoresis, samples were incubated with 1 mM PAA ˜8 kDa for 1 hr at room temperature to help improve analysis by gel electrophoresis.

The following were purchased from: spermidine (Cayman Chemical, Product #14918).

### Example 106

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 52 C and D.
M=PCR Marker
1. 50 mM Na-Acetate (pH 5.2)
2. 250 mM NaCl+50 mM Na-Acetate (pH 5.2)
3. 500 mM NaCl+50 mM Na-Acetate (pH 5.2)
4. 750 mM NaCl+50 mM Na-Acetate (pH 5.2)
5. 1M NaCl+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 52 C and D.

Prior to analysis by agarose gel electrophoresis, samples were incubated with 1 mM PAA ˜8 kDa for 1 hr at room temperature to help improve analysis by gel electrophoresis. Sodium Chloride (NaCl) (Sigma-Aldrich, Product #S3014)

### Example 107

### Accelerated RNA Stability Testing at 60° C.

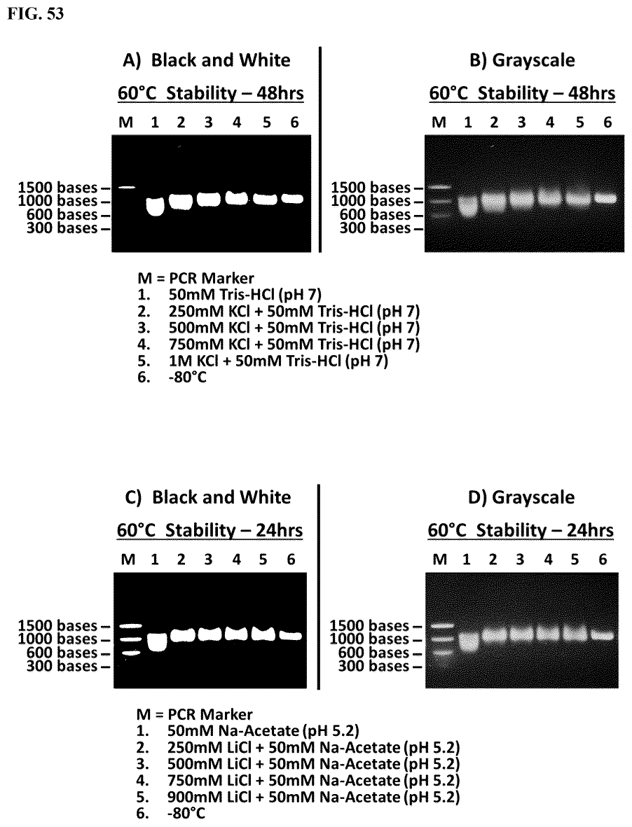

In vitro transcribed RNA was diluted at a ratio of about 1:10 (about 100-300 μg/mL) in different compositions containing either 50 mM Tris-HCl (pH 7), or one or more compounds in compositions with 50 mM Tris-HCl (pH 7) as shown in FIGS. 53A and B.
M=PCR Marker
1. 50 mM Tris-HCl (pH 7)
2. 250 mM KCl+50 mM Tris-HCl (pH 7)
3. 500 mM KCl+50 mM Tris-HCl (pH 7)
4. 750 mM KCl+50 mM Tris-HCl (pH 7)
5. 1M KCl+50 mM Tris-HCl (pH 7)
6. −80° C.

Following dilution of each sample in each respective RNA storage environment, samples were stored in a thermal cycler at 60° C. for about 48 hours and then analyzed by denaturing agarose gel electrophoresis as shown in FIGS. 53A and B.

Prior to analysis by agarose gel electrophoresis, samples were incubated with 1 mM PAA ˜8 kDa for 1 hr at room temperature to help improve analysis by gel electrophoresis.

Potassium Chloride (KCl) (Sigma-Aldrich, Product #P9541)

### Example 108

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 53 C and D.
M=PCR Marker
1. 50 mM Na-Acetate (pH 5.2)
2. 250 mM LiCl+50 mM Na-Acetate (pH 5.2)
3. 500 mM LiCl+50 mM Na-Acetate (pH 5.2)
4. 750 mM LiCl+50 mM Na-Acetate (pH 5.2)
5. 900 mM LiCl+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 53 C and D.

Prior to analysis by agarose gel electrophoresis, samples were incubated with 1 mM PAA ˜8 kDa for 1 hr at room temperature to help improve analysis by gel electrophoresis.

Lithium Chloride (LiCl), Lithium Chloride Solution (New England Biolabs, as component B2051AVIAL in Product #E2050S).

### Example 109

### Accelerated RNA Stability Testing at 60° C.

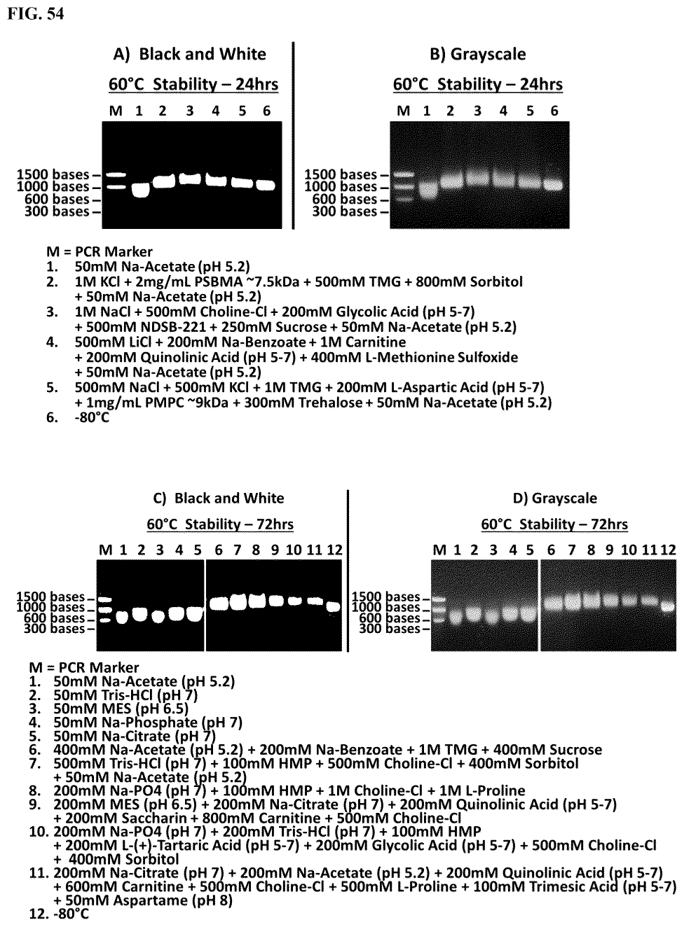

In vitro transcribed RNA was diluted at a ratio of about 1:10 (about 100-300 μg/mL) in different compositions containing either 50 mM Na-Acetate (pH 5.2), or one or more compounds in compositions with 50 mM Na-Acetate (pH 5.2) as shown in FIGS. 54A and B.
M=PCR Marker
1. 50 mM Na-Acetate (pH 5.2)
2. 1M KCl+2 mg/mL PSBMA ˜7.5 kDa+500 mM TMG+800 mM Sorbitol+50 mM Na-Acetate (pH 5.2)
3. 1M NaCl+500 mM Choline-Cl+200 mM Glycolic Acid (pH 5-7)+500 mM NDSB-221+250 mM Sucrose+50 mM Na-Acetate (pH 5.2)
4. 500 mM LiCl+200 mM Na-Benzoate+1M L-Carnitine+200 mM Quinolinic Acid (pH 5-7)+400 mM L-Methionine Sulfoxide+50 mM Na-Acetate (pH 5.2)
5. 500 mM NaCl+500 mM KCl+1M TMG+200 mM L-Aspartic Acid (pH 5-7)+1 mg/mL PMPC ˜9 kDa+300 mM Trehalose+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 54A and B.

Prior to analysis by agarose gel electrophoresis, samples were incubated with 1 mM PAA ˜8 kDa for 1 hr at room temperature to help improve analysis by gel electrophoresis.

### Example 110

### Accelerated RNA Stability Testing at 60° C.

In vitro transcribed RNA was diluted at a ratio of about 1:10 (about 100-300 μg/mL) in different compositions containing either 50 mM Na-Acetate (pH 5.2), 50 mM Tris-HCl (pH 7), 50 mM MES buffer (pH 6.5), 50 mM Na-Phosphate (pH 7), 50 mM Na-Citrate (pH 7) or various combinations of more than one RNA stabilizing substance with selected buffers as shown in FIGS. 54 C and D.
M=PCR Marker
1. 50 mM Na-Acetate (pH 5.2)
2. 50 mM Tris-HCl (pH 7)
3. 50 mM MES (pH 6.5)
4. 50 mM Na-Phosphate (pH 7)
5. 50 mM Na-Citrate (pH 7)
6. 400 mM Na-Acetate (pH 5.2)+200 mM Na-Benzoate+1M TMG+400 mM Sucrose
7. 500 mM Tris-HCl (pH 7)+100 mM HMP+500 mM Choline-Cl+400 mM Sorbitol+50 mM Na-Acetate (pH 5.2)
8. 200 mM Na—PO4 (pH 7)+100 mM HMP+1M Choline-Cl+1M L-Proline
9. 200 mM MES (pH 6.5)+200 mM Na-Citrate (pH 7)+200 mM Quinolinic Acid (pH 5-7)+200 mM Saccharin+800 mM L-Carnitine+500 mM Choline-C₁
10. 200 mM Na—PO4 (pH 7)+200 mM Tris-HCl (pH 7)+100 mM HMP+200 mM L-(+)-Tartaric Acid (pH 5-7)+200 mM Glycolic Acid (pH 5-7)+500 mM Choline-Cl+400 mM Sorbitol
11. 200 mM Na-Citrate (pH 7)+200 mM Na-Acetate (pH 5.2)+200 mM Quinolinic Acid (pH 5-7)+600 mM L-Carnitine+500 mM Choline-Cl+500 mM L-Proline+100 mM Trimesic Acid (pH 5-7)+50 mM Aspartame (pH 8)
12. −80° C.

Following dilution of each sample in each respective RNA storage environment, samples were stored in a thermal cycler at 60° C. for about 72 hours and then analyzed by denaturing agarose gel electrophoresis as shown in FIGS. 54 C and D.

### Example 111

### Accelerated RNA Stability Testing at 60° C.

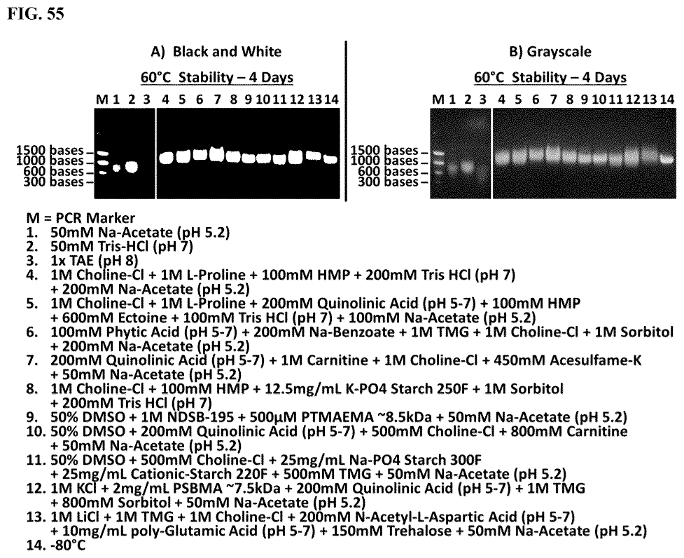

In vitro transcribed RNA was diluted at a ratio of about 1:10 (about 100-300 μg/mL) in different compositions containing either 50 mM Na-Acetate (pH 5.2), 50 mM Tris-HCl (pH 7), TAE buffer (pH 8) or various combinations of more than one RNA stabilizing substance with selected buffers as shown in FIGS. 55A and B.
M=PCR Marker
1. 50 mM Na-Acetate (pH 5.2)
2. 50 mM Tris-HCl (pH 7)
3. 1×TAE (pH 8)
4. 1M Choline-Cl+1M L-Proline+100 mM HMP+200 mM Tris HCl (pH 7)+200 mM Na-Acetate (pH 5.2)
5. 1M Choline-Cl+1M L-Proline+200 mM Quinolinic Acid (pH 5-7)+100 mM HMP+600 mM Ectoine+100 mM Tris HCl (pH 7)+100 mM Na-Acetate (pH 5.2)
6. 100 mM Phytic Acid (pH 5-7)+200 mM Na-Benzoate+1M TMG+1M Choline-Cl+1M Sorbitol+200 mM Na-Acetate (pH 5.2)
7. 200 mM Quinolinic Acid (pH 5-7)+1M Carnitine+1M Choline-Cl+450 mM Acesulfame-K+50 mM Na-Acetate (pH 5.2)
8. 1M Choline-Cl+100 mM HMP+12.5 mg/mL K—PO4 Starch 250F+1M Sorbitol+200 mM Tris HCl (pH 7)
9. 50% DMSO+1M NDSB-195+500 μM PTMAEMA ˜8.5 kDa+50 mM Na-Acetate (pH 5.2)
10. 50% DMSO+200 mM Quinolinic Acid (pH 5-7)+500 mM Choline-Cl+800 mM Carnitine +50 mM Na-Acetate (pH 5.2)
11. 50% DMSO+500 mM Choline-Cl+25 mg/mL Na—PO4 Starch 300F+25 mg/mL Cationic-Starch 220F+500 mM TMG+50 mM Na-Acetate (pH 5.2)
12. 1M KCl+2 mg/mL PSBMA ˜7.5 kDa+200 mM Quinolinic Acid (pH 5-7)+1M TMG+800 mM Sorbitol+50 mM Na-Acetate (pH 5.2)
13. 1M LiCl+1M TMG+1M Choline-Cl+200 mM N-Acetyl-L-Aspartic Acid (pH 5-7)+10 mg/mL poly-Glutamic Acid (pH 5-7)+150 mM Trehalose+50 mM Na-Acetate (pH 5.2)
14. −80° C.

Following dilution of each sample in each respective RNA storage environment, samples were stored in a thermal cycler at 60° C. for about 4 days and then analyzed by denaturing agarose gel electrophoresis as shown in FIGS. 55A and B.

### Example 112

### Accelerated RNA Stability Testing at 60° C.

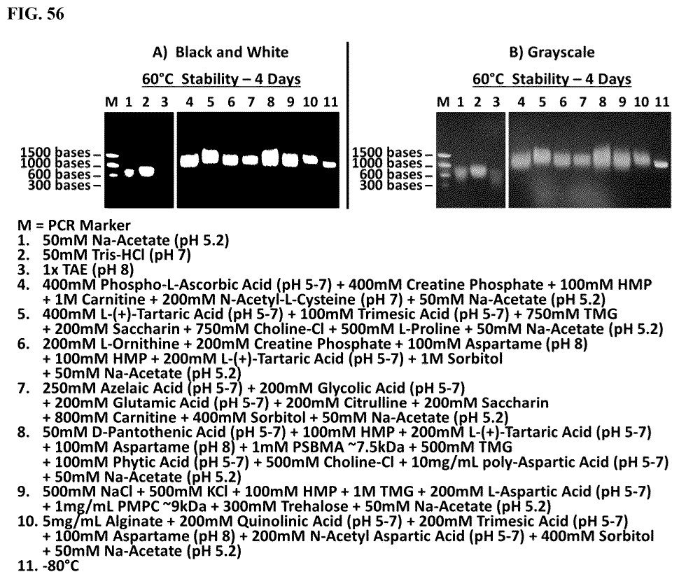

In vitro transcribed RNA was diluted at a ratio of about 1:10 (about 100-300 μg/mL) in different compositions containing either 50 mM Na-Acetate (pH 5.2), 50 mM Tris-HCl (pH 7), TAE buffer (pH 8) or various combinations of more than one RNA stabilizing substance with selected buffers as shown in FIGS. 56A and B.
M=PCR Marker
1. 50 mM Na-Acetate (pH 5.2)
2. 50 mM Tris-HCl (pH 7)
3. 1×TAE (pH 8)
4. 400 mM Phospho-L-Ascorbic Acid (pH 5-7)+400 mM Creatine Phosphate+100 mM HMP+1M L-Carnitine+200 mM N-Acetyl-L-Cysteine (pH 7)+50 mM Na-Acetate (pH 5.2)
5. 400 mM L-(+)-Tartaric Acid (pH 5-7)+100 mM Trimesic Acid (pH 5-7)+750 mM TMG+200 mM Saccharin+750 mM Choline-Cl+500 mM L-Proline+50 mM Na-Acetate (pH 5.2)
6. 200 mM L-Ornithine+200 mM Creatine Phosphate+100 mM Aspartame (pH 8)+100 mM HMP+200 mM L-(+)-Tartaric Acid (pH 5-7)+1M Sorbitol+50 mM Na-Acetate (pH 5.2)
7. 250 mM Azelaic Acid (pH 5-7)+200 mM Glycolic Acid (pH 5-7)+200 mM Glutamic Acid (pH 5-7)+200 mM Citrulline+200 mM Saccharin+800 mM L-Carnitine+400 mM Sorbitol+50 mM Na-Acetate (pH 5.2)
8. 50 mM D-Pantothenic Acid (pH 5-7)+100 mM HMP+200 mM L-(+)-Tartaric Acid (pH 5-7)+100 mM Aspartame (pH 8)+1 mM PSBMA ˜7.5 kDa+500 mM TMG+100 mM Phytic Acid (pH 5-7)+500 mM Choline-Cl+10 mg/mL poly-Aspartic Acid (pH 5-7)+50 mM Na-Acetate (pH 5.2)
9. 500 mM NaCl+500 mM KCl+100 mM HMP+1M TMG+200 mM L-Aspartic Acid (pH 5-7)+1 mg/mL PMPC ˜9 kDa+300 mM Trehalose+50 mM Na-Acetate (pH 5.2)
10. 5 mg/mL Alginate+200 mM Quinolinic Acid (pH 5-7)+200 mM Trimesic Acid (pH 5-7)+100 mM Aspartame (pH 8)+200 mM N-Acetyl Aspartic Acid (pH 5-7)+400 mM Sorbitol+50 mM Na-Acetate (pH 5.2)
11. −80° C.

Following dilution of each sample in each respective RNA storage environment, samples were stored in a thermal cycler at 60° C. for about 4 days and then analyzed by denaturing agarose gel electrophoresis as shown in FIGS. 56A and B.

### Example 113

### Accelerated RNA Stability Testing at 40° C.

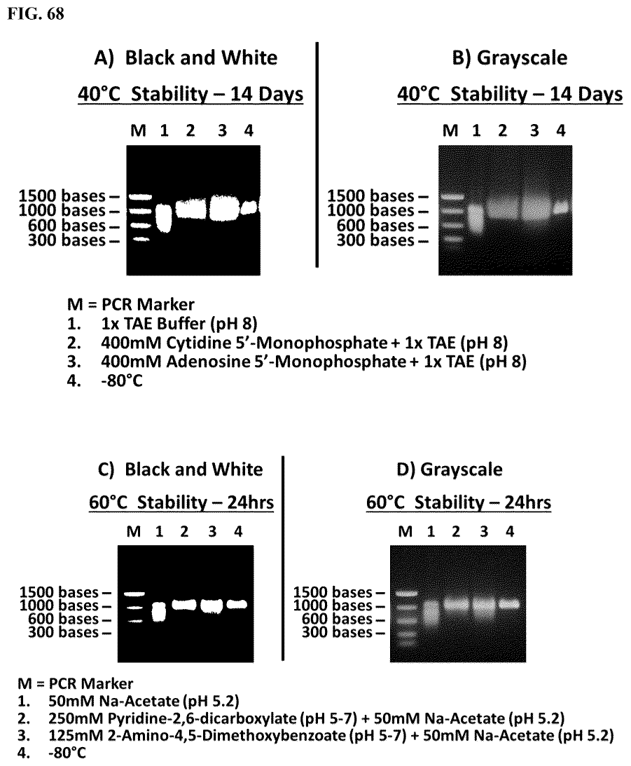

In vitro transcribed RNA was diluted at a ratio of about 1:10 (about 100-300 μg/mL) in different compositions containing TAE buffer (pH 8) or one or more compounds in compositions with TAE buffer (pH 8) as shown in FIGS. 68A and B.
M=PCR Marker
1. 1×TAE Buffer (pH 8)
2. 400 mM Cytidine 5′-Monophosphate+1×TAE (pH 8)
3. 400 mM Adenosine 5′-Monophosphate+1×TAE (pH 8)
4. −80° C.

Following dilution of each sample in each respective RNA storage environment, samples were stored in a thermal cycler at 40° C. for about 14 days and then analyzed by denaturing agarose gel electrophoresis as shown in FIGS. 68A and B.

The following substances in one or more compositions were purchased as follows: Cytidine 5′-Monophosphate (Cayman Chemical, Product #35116), Adenosine 5′-Monophosphate (Cayman Chemical, Product #21094).

### Example 114

### Accelerated RNA Stability Testing at 60° C.

In vitro transcribed RNA was diluted at a ratio of about 1:10 (about 100-300 μg/mL) in different compositions containing 50 mM Na-Acetate (pH 5.2) or one or more compounds in compositions with 50 mM Na-Acetate (pH 5.2) as shown in FIGS. 68 C and D.
M=PCR Marker
1. 50 mM Na-Acetate (pH 5.2)
2. 250 mM Pyridine-2,6-dicarboxylate (pH 5-7)+50 mM Na-Acetate (pH 5.2)
3. 125 mM 2-Amino-4,5-Dimethoxybenzoate (pH 5-7)+50 mM Na-Acetate (pH 5.2)
4. −80° C.

Following dilution of each sample in each respective RNA storage environment, samples were stored in a thermal cycler at 60° C. for about 24 hours and then analyzed by denaturing agarose gel electrophoresis as shown in FIGS. 68 C and D.

Prior to analysis by agarose gel electrophoresis, sample 3 was buffer exchanged to remove 2-amino-4,5-dimethoxybenzoate to help improve analysis by gel electrophoresis.

The following substances in one or more compositions were purchased as follows: Pyridine-2,6-dicarboxylate (Sigma-Aldrich, Product #02321), 2-Amino-4,5-Dimethoxybenzoate (Sigma-Aldrich, Product #252042).

### Example 115

### Accelerated RNA Stability Testing at 60° C.

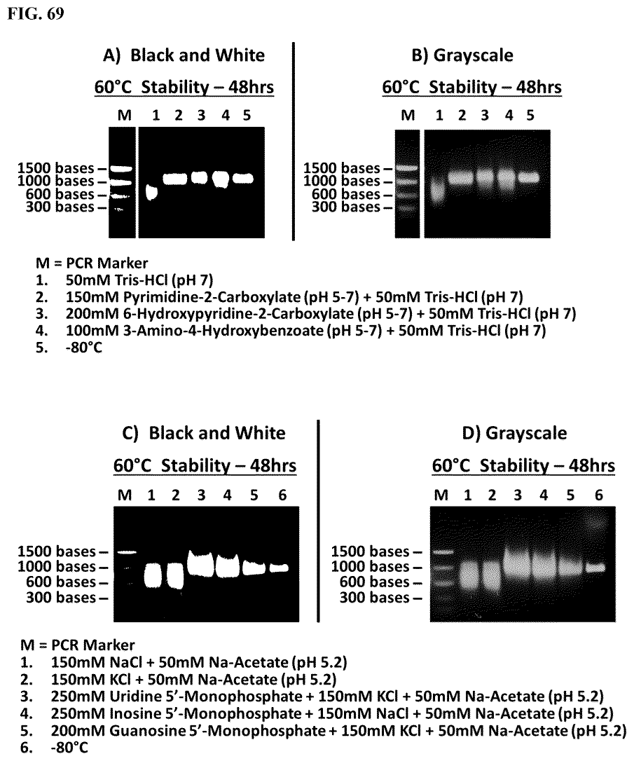

In vitro transcribed RNA was diluted at a ratio of about 1:10 (about 100-300 μg/mL) in different compositions containing 50 mM Tris-HCl (pH 7) or one or more compounds in compositions with 50 mM Tris-HCl (pH 7) as shown in FIGS. 69A and B.
M=PCR Marker
1. 50 mM Tris-HCl (pH 7)
2. 150 mM Pyrimidine-2-Carboxylate (pH 5-7)+50 mM Tris-HCl (pH 7)
3. 200 mM 6-Hydroxypyridine-2-Carboxylate (pH 5-7)+50 mM Tris-HCl (pH 7)
4. 100 mM 3-Amino-4-Hydroxybenzoate (pH 5-7)+50 mM Tris-HCl (pH 7)
5. −80° C.

Following dilution of each sample in each respective RNA storage environment, samples were stored in a thermal cycler at 60° C. for about 48 hours and then analyzed by denaturing agarose gel electrophoresis as shown in FIGS. 69A and B.

Prior to analysis by agarose gel electrophoresis, samples 3 and 4 were buffer exchanged to remove 6-hydroxypyridine-2-carboxylate and 3-amino-4-hydroxybenzoate to help improve analysis by gel electrophoresis.

The following substances in one or more compositions were purchased as follows: Pyrimidine-2-Carboxylate (Sigma-Aldrich, Product #754315), 6-Hydroxypyridine-2-Carboxylate (Sigma-Aldrich, Product #384305), 3-Amino-4-Hydroxybenzoate (Sigma-Aldrich, Product #289647).

### Example 116

### Accelerated RNA Stability Testing at 60° C.

In vitro transcribed RNA was diluted at a ratio of about 1:10 (about 100-300 μg/mL) in different compositions containing 150 mM NaCl+50 mM Na-Acetate (pH 5.2) or 150 mM KCl+50 mM Na-Acetate (pH 5.2) or one or more compounds in compositions with 150 mM NaCl+50 mM Na-Acetate (pH 5.2) or 150 mM KCl+50 mM Na-Acetate (pH 5.2) as shown in FIGS. 69 C and D.
M=PCR Marker
1. 150 mM NaCl+50 mM Na-Acetate (pH 5.2)
2. 150 mM KCl+50 mM Na-Acetate (pH 5.2)
3. 250 mM Uridine 5′-Monophosphate+150 mM KCl+50 mM Na-Acetate (pH 5.2)
4. 250 mM Inosine 5′-Monophosphate+150 mM NaCl+50 mM Na-Acetate (pH 5.2)
5. 200 mM Guanosine 5′-Monophosphate+150 mM KCl+50 mM Na-Acetate (pH 5.2)
6. −80° C.

Following dilution of each sample in each respective RNA storage environment, samples were stored in a thermal cycler at 60° C. for about 48 hours and then analyzed by denaturing agarose gel electrophoresis as shown in FIGS. 69 C and D.

Prior to analysis by agarose gel electrophoresis, sample 5 with guanosine-5′-monophosphate was diluted 2× with water and adjusted to pH 7 with 1M Tris-HCl (pH 7) to help improve analysis by gel electrophoresis.

The following substances in one or more compositions were purchased as follows: Uridine 5′-Monophosphate (Liftmode operated by Synaptent LLC, Chicago, IL), Inosine 5′-Monophosphate (Cayman Chemical, Product #18135), Guanosine 5′-Monophosphate (Cayman Chemical, Product #16957).

### Example 117

### Accelerated RNA Stability Testing at 60° C.

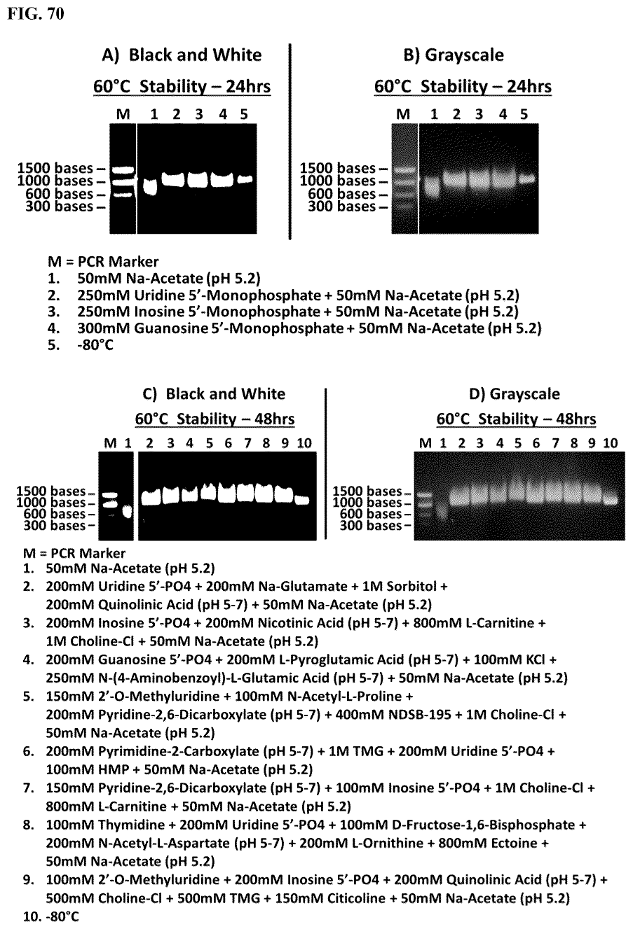

In vitro transcribed RNA was diluted at a ratio of about 1:10 (about 100-300 μg/mL) in different compositions containing 50 mM Na-Acetate (pH 5.2) or one or more compounds in compositions with 50 mM Na-Acetate (pH 5.2) as shown in FIGS. 70A and B.
M=PCR Marker
1. 50 mM Na-Acetate (pH 5.2)
2. 250 mM Uridine 5′-Monophosphate+50 mM Na-Acetate (pH 5.2)
3. 250 mM Inosine 5′-Monophosphate+50 mM Na-Acetate (pH 5.2)
4. 300 mM Guanosine 5′-Monophosphate+50 mM Na-Acetate (pH 5.2)
5. −80° C.

Following dilution of each sample in each respective RNA storage environment, samples were stored in a thermal cycler at 60° C. for about 24 hours and then analyzed by denaturing agarose gel electrophoresis as shown in FIGS. 70A and B.

Prior to analysis by agarose gel electrophoresis, sample 4 with guanosine-5′-monophosphate was diluted 2× with water and adjusted to pH 7 with 1M Tris-HCl (pH 7) to help improve analysis by gel electrophoresis.

### Example 118

### Accelerated RNA Stability Testing at 60° C.

In vitro transcribed RNA was diluted at a ratio of about 1:10 (about 100-300 μg/mL) in different compositions containing 50 mM Na-Acetate (pH 5.2) or one or more compounds in compositions with 50 mM Na-Acetate (pH 5.2) as shown in FIGS. 70 C and D.
M=PCR Marker
1. 50 mM Na-Acetate (pH 5.2)
2. 200 mM Uridine 5′-PO4+200 mM Na-Glutamate+1M Sorbitol+200 mM Quinolinic Acid (pH 5-7)+50 mM Na-Acetate (pH 5.2)
3. 200 mM Inosine 5′-PO4+200 mM Nicotinic Acid (pH 5-7)+800 mM L-Carnitine+1M Choline-Cl+50 mM Na-Acetate (pH 5.2)
4. 200 mM Guanosine 5′-PO4+200 mM L-Pyroglutamic Acid (pH 5-7)+100 mM KCl+250 mM N-(4-Aminobenzoyl)-L-Glutamic Acid (pH 5-7)+50 mM Na-Acetate (pH 5.2)
5. 150 mM 2′-O-Methyluridine+100 mM N-Acetyl-L-Proline+200 mM Pyridine-2,6-Dicarboxylate (pH 5-7)+400 mM NDSB-195+1M Choline-Cl+50 mM Na-Acetate (pH 5.2)
6. 200 mM Pyrimidine-2-Carboxylate (pH 5-7)+1M TMG+200 mM Uridine 5′-PO₄₊₁₀₀ mM HMP+50 mM Na-Acetate (pH 5.2)
7. 150 mM Pyridine-2,6-Dicarboxylate (pH 5-7)+100 mM Inosine 5′-PO4+1M Choline-Cl+800 mM L-Carnitine+50 mM Na-Acetate (pH 5.2)
8. 100 mM Thymidine+200 mM Uridine 5′-PO4+100 mM D-Fructose-1,6-Bisphosphate+200 mM N-Acetyl-L-Aspartate (pH 5-7)+200 mM L-Ornithine+800 mM Ectoine+50 mM Na-Acetate (pH 5.2)
9. 100 mM 2′-O-Methyluridine+200 mM Inosine 5′-PO4+200 mM Quinolinic Acid (pH 5-7)+500 mM Choline-Cl+500 mM TMG+150 mM Citicoline+50 mM Na-Acetate (pH 5.2)
10. −80° C.

Following dilution of each sample in each respective RNA storage environment, samples were stored in a thermal cycler at 60° C. for about 48 hours and then analyzed by denaturing agarose gel electrophoresis as shown in FIGS. 70 C and D.

Prior to analysis by agarose gel electrophoresis, samples were diluted 2× with water and samples with guanosine-5′-monophosphate were adjusted to pH 7 with 1M Tris-HCl (pH 7) to help improve analysis by gel electrophoresis.

The following substances in one or more compositions were purchased as follows: L-Pyroglutamic Acid (Nootropics Depot, Tempe, AZ)

### Example 119

### Accelerated RNA Stability Testing at 60° C.

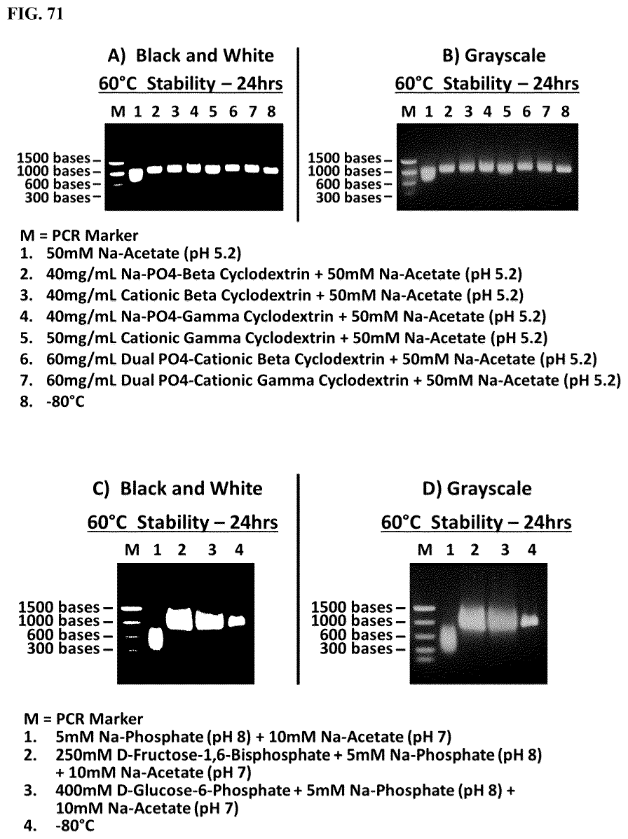

In vitro transcribed RNA was diluted at a ratio of about 1:10 (about 100-300 μg/mL) in different compositions containing 50 mM Na-Acetate (pH 5.2) or one or more compounds in compositions with 50 mM Na-Acetate (pH 5.2) as shown in FIGS. 71A and B.
M=PCR Marker
1. 50 mM Na-Acetate (pH 5.2)
2. 40 mg/mL Na—PO4-Beta Cyclodextrin+50 mM Na-Acetate (pH 5.2)
3. 40 mg/mL Cationic Beta Cyclodextrin+50 mM Na-Acetate (pH 5.2)
4. 40 mg/mL Na—PO4-Gamma Cyclodextrin+50 mM Na-Acetate (pH 5.2)
5. 50 mg/mL Cationic Gamma Cyclodextrin+50 mM Na-Acetate (pH 5.2)
6. 60 mg/mL Dual PO4-Cationic Beta Cyclodextrin+50 mM Na-Acetate (pH 5.2)
7. 60 mg/mL Dual PO4-Cationic Gamma Cyclodextrin+50 mM Na-Acetate (pH 5.2)
8. −80° C.

Following dilution of each sample in each respective RNA storage environment, samples were stored in a thermal cycler at 60° C. for about 24 hours and then analyzed by denaturing agarose gel electrophoresis as shown in FIGS. 71A and B.

Prior to analysis by agarose gel electrophoresis, samples 3 and 5 with cationic beta cyclodextrin and cationic gamma cyclodextrin were incubated with 2.5 mM PAA ˜8 kDa for 1 hr at room temperature to help improve analysis by agarose gel electrophoresis.

Na—PO4-Beta Cyclodextrin, Cationic Beta Cyclodextrin, Na—PO4-Gamma Cyclodextrin, Cationic Gamma Cyclodextrin, Dual PO4-Cationic Beta Cyclodextrin, Dual PO4-Cationic Gamma Cyclodextrin modified carbohydrates are described above in Example 5-A and Table 4-2, Table 5-2, and Table 5-3.

### Example 120

### Accelerated RNA Stability Testing at 60° C.

In vitro transcribed RNA was diluted at a ratio of about 1:10 (about 100-300 μg/mL) in different compositions containing 5 mM Na-Phosphate (pH 8)+10 mM Na-Acetate (pH 7) or one or more compounds in compositions with 5 mM Na-Phosphate (pH 8)+10 mM Na-Acetate (pH 7) as shown in FIGS. 71 C and D.
M=PCR Marker
2. 250 mM D-Fructose-1,6-Bisphosphate+5 mM Na-Phosphate (pH 8)+10 mM Na-Acetate (pH 7)
3. 400 mM D-Glucose-6-Phosphate+5 mM Na-Phosphate (pH 8)+10 mM Na-Acetate (pH 7)
4. −80° C.

Following dilution of each sample in each respective RNA storage environment, samples were stored in a thermal cycler at 60° C. for about 24 hours and then analyzed by denaturing agarose gel electrophoresis as shown in FIGS. 71 C and D.

The following substances in one or more compositions were purchased as follows: D-Fructose-1,6-Bisphosphate (Cayman Chemical, Product #20516), D-Glucose-6-Phosphate (Cayman Chemical, Product #20376).

### Example 121

### Accelerated RNA Stability Testing at 50° C.

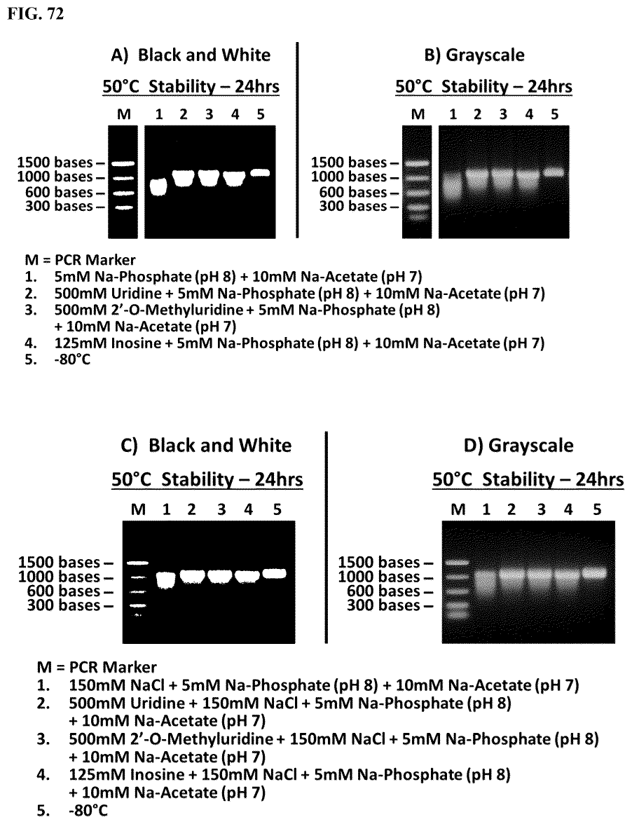

In vitro transcribed RNA was diluted at a ratio of about 1:10 (about 100-300 μg/mL) in different compositions containing 5 mM Na-Phosphate (pH 8)+10 mM Na-Acetate (pH 7) or one or more compounds in compositions with 5 mM Na-Phosphate (pH 8)+10 mM Na-Acetate (pH 7) as shown in FIGS. 72A and B.
M=PCR Marker
1. 5 mM Na-Phosphate (pH 8)+10 mM Na-Acetate (pH 7)
2. 500 mM Uridine+5 mM Na-Phosphate (pH 8)+10 mM Na-Acetate (pH 7)
3. 500 mM 2′-O-Methyluridine+5 mM Na-Phosphate (pH 8)+10 mM Na-Acetate (pH 7)
4. 125 mM Inosine+5 mM Na-Phosphate (pH 8)+10 mM Na-Acetate (pH 7)
5. −80° C.

Following dilution of each sample in each respective RNA storage environment, samples were stored in a thermal cycler at 50° C. for about 24 hours and then analyzed by denaturing agarose gel electrophoresis as shown in FIGS. 72A and B.

The following substances in one or more compositions were purchased as follows: Uridine (Cayman Chemical, Product #20300), 2′-O-Methyluridine (Cayman Chemical, Product #36520), 125 mM Inosine (Cayman Chemical, Product #34373).

### Example 122

### Accelerated RNA Stability Testing at 50° C.

In vitro transcribed RNA was diluted at a ratio of about 1:10 (about 100-300 μg/mL) in different compositions containing 150 mM NaCl+5 mM Na-Phosphate (pH 8)+10 mM Na-Acetate (pH 7) or one or more compounds in compositions with 150 mM NaCl+5 mM Na-Phosphate (pH 8)+10 mM Na-Acetate (pH 7) as shown in FIGS. 72 C and D.
M=PCR Marker
1. 150 mM NaCl+5 mM Na-Phosphate (pH 8)+10 mM Na-Acetate (pH 7)
2. 500 mM Uridine+150 mM NaCl+5 mM Na-Phosphate (pH 8)+10 mM Na-Acetate (pH 7)
3. 500 mM 2′-O-Methyluridine+150 mM NaCl+5 mM Na-Phosphate (pH 8)+10 mM Na-Acetate (pH 7)
4. 125 mM Inosine+150 mM NaCl+5 mM Na-Phosphate (pH 8)+10 mM Na-Acetate (pH 7)
5. −80° C.

Following dilution of each sample in each respective RNA storage environment, samples were stored in a thermal cycler at 50° C. for about 24 hours and then analyzed by denaturing agarose gel electrophoresis as shown in FIGS. 72 C and D.

### Example 123

### Accelerated RNA Stability Testing at 60° C.

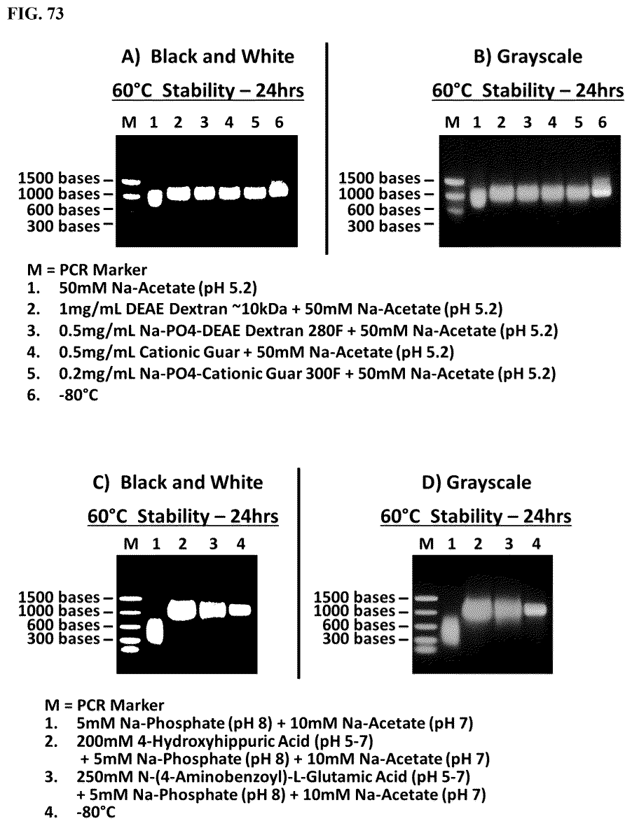

In vitro transcribed RNA was diluted at a ratio of about 1:10 (about 100-300 μg/mL) in different compositions containing 50 mM Na-Acetate (pH 5.2) or one or more compounds in compositions with 50 mM Na-Acetate (pH 5.2) as shown in FIGS. 73A and B.
M=PCR Marker
1. 50 mM Na-Acetate (pH 5.2)
2. 1 mg/mL DEAE Dextran ˜10 kDa+50 mM Na-Acetate (pH 5.2)
3. 0.5 mg/mL Na—PO4-DEAE Dextran 280F+50 mM Na-Acetate (pH 5.2)
4. 0.5 mg/mL Cationic Guar+50 mM Na-Acetate (pH 5.2)
5. 0.2 mg/mL Na—PO4-Cationic Guar 300F+50 mM Na-Acetate (pH 5.2)
6. −80° C.

Following dilution of each sample in each respective RNA storage environment, samples were stored in a thermal cycler at 60° C. for about 24 hours and then analyzed by denaturing agarose gel electrophoresis as shown in FIGS. 73A and B.

Prior to analysis by agarose gel electrophoresis, samples were incubated with 1.5 mM PAA ˜8 kDa for 1 hr at room temperature and then diluted 2× with water to help improve analysis by agarose gel electrophoresis.

Na—PO4-DEAE Dextran 280F and Na—PO4-Cationic Guar 300F modified carbohydrates are described above in Example 5-A and Table 4-2.

The following substances in one or more compositions were purchased as follows: Diethylaminoethyl-Dextran ˜10 kDa (DEAE-Dextran) (Sigma-Aldrich Product, #00898), Guar Hydroxypropyltrimonium Chloride (Cationic Guar, GuarCat) (Lotioncrafters, Eastsound, WA).

### Example 124

### Accelerated RNA Stability Testing at 60° C.

In vitro transcribed RNA was diluted at a ratio of about 1:10 (about 100-300 μg/mL) in different compositions containing 5 mM Na-Phosphate (pH 8)+10 mM Na-Acetate (pH 7) or one or more compounds in compositions with 5 mM Na-Phosphate (pH 8)+10 mM Na-Acetate (pH 7) as shown in FIGS. 73 C and D.
M=PCR Marker
1. 5 mM Na-Phosphate (pH 8)+10 mM Na-Acetate (pH 7)
2. 200 mM 4-Hydroxyhippuric Acid (pH 5-7)+5 mM Na-Phosphate (pH 8)+10 mM Na-Acetate (pH 7)
3. 250 mM N-(4-Aminobenzoyl)-L-Glutamic Acid (pH 5-7)+5 mM Na-Phosphate (pH 8)+10 mM Na-Acetate (pH 7)
4. −80° C.

Following dilution of each sample in each respective RNA storage environment, samples were stored in a thermal cycler at 60° C. for about 24 hours and then analyzed by denaturing agarose gel electrophoresis as shown in FIGS. 73 C and D.

Prior to analysis by agarose gel electrophoresis, sample 2 was buffer exchanged to remove 4-hydroxyhippuric acid (pH 5-7) to help improve analysis by agarose gel electrophoresis.

The following substances in one or more compositions were purchased as follows: 4-Hydroxyhippuric Acid (Cayman Chemical, Product #30115), N-(4-Aminobenzoyl)-L-Glutamic Acid (Cayman Chemical, Product #33919).

### Example 125

### Accelerated RNA Stability Testing at 50° C.

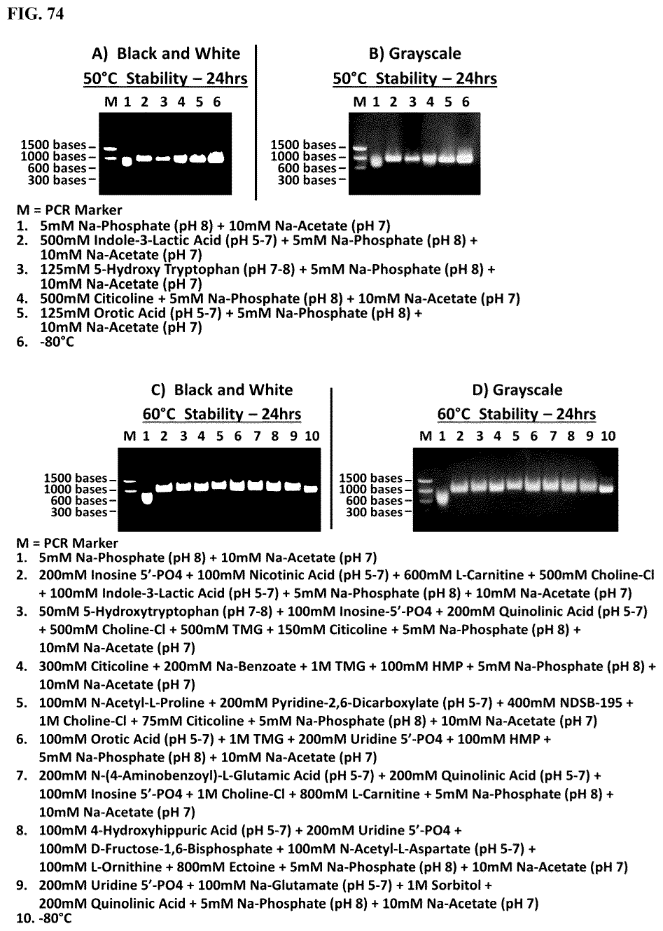

In vitro transcribed RNA was diluted at a ratio of about 1:10 (about 100-300 μg/mL) in different compositions containing 5 mM Na-Phosphate (pH 8)+10 mM Na-Acetate (pH 7) or one or more compounds in compositions with 5 mM Na-Phosphate (pH 8)+10 mM Na-Acetate (pH 7) as shown in FIGS. 74A and B.
M=PCR Marker
1. 5 mM Na-Phosphate (pH 8)+10 mM Na-Acetate (pH 7)
2. 500 mM Indole-3-Lactic Acid (pH 5-7)+5 mM Na-Phosphate (pH 8)+10 mM Na-Acetate (pH 7)
3. 125 mM 5-Hydroxy Tryptophan (pH 7-8)+5 mM Na-Phosphate (pH 8)+10 mM Na-Acetate (pH 7)
4. 500 mM Citicoline+5 mM Na-Phosphate (pH 8)+10 mM Na-Acetate (pH 7)
5. 125 mM Orotic Acid (pH 5-7)+5 mM Na-Phosphate (pH 8)+10 mM Na-Acetate (pH 7)
6. −80° C.

Following dilution of each sample in each respective RNA storage environment, samples were stored in a thermal cycler at 50° C. for about 24 hours and then analyzed by denaturing agarose gel electrophoresis as shown in FIGS. 74A and B.

Orotic acid was adjusted to (pH 5-7) with NaOH and added to the composition as a suspension of orotate in water at the specified concentration. Prior to analysis by agarose gel electrophoresis, samples were diluted 2× with water and sample 5 with orotic acid was centrifuged to remove residual undissolved material to help improve analysis by gel electrophoresis. The agarose gel was also imaged at about 470-490 nm to help improve sample analysis.

The following substances in one or more compositions were purchased as follows: Indole-3-Lactic Acid (Cayman Chemical, Product #37534), 5-Hydroxy Tryptophan (5-HTP) (Liftmode, operated by Synaptent, LLC), Citicoline (Liftmode, operated by Synaptent, LLC), Orotic Acid (Bulk Supplements, Henderson, NV, SKU: ORO500).

### Example 126

### Accelerated RNA Stability Testing at 60° C.

In vitro transcribed RNA was diluted at a ratio of about 1:10 (about 100-300 μg/mL) in different compositions containing 5 mM Na-Phosphate (pH 8)+10 mM Na-Acetate (pH 7) or one or more compounds in compositions with 5 mM Na-Phosphate (pH 8)+10 mM Na-Acetate (pH 7) as shown in FIGS. 74 C and D.
M=PCR Marker
1. 5 mM Na-Phosphate (pH 8)+10 mM Na-Acetate (pH 7)
2. 200 mM Inosine 5′-PO4+100 mM Nicotinic Acid (pH 5-7)+600 mM L-Carnitine+500 mM Choline-Cl+100 mM Indole-3-Lactic Acid (pH 5-7)+5 mM Na-Phosphate (pH 8)+10 mM Na-Acetate (pH 7)
3. 50 mM 5-Hydroxytryptophan (pH 7-8)+100 mM Inosine-5′-PO4+200 mM Quinolinic Acid (pH 5-7)+500 mM Choline-Cl+500 mM TMG+150 mM Citicoline+5 mM Na-Phosphate (pH 8)+10 mM Na-Acetate (pH 7)
4. 300 mM Citicoline+200 mM Na-Benzoate+1M TMG+100 mM HMP+5 mM Na-Phosphate (pH 8)+10 mM Na-Acetate (pH 7)
5. 100 mM N-Acetyl-L-Proline+200 mM Pyridine-2,6-Dicarboxylate (pH 5-7)+400 mM NDSB-195+1M Choline-Cl+75 mM Citicoline+5 mM Na-Phosphate (pH 8)+10 mM Na-Acetate (pH 7)
6. 100 mM Orotic Acid (pH 5-7)+1M TMG+200 mM Uridine 5′-PO4+100 mM HMP+5 mM Na-Phosphate (pH 8)+10 mM Na-Acetate (pH 7)
7. 200 mM N-(4-Aminobenzoyl)-L-Glutamic Acid (pH 5-7)+200 mM Quinolinic Acid (pH 5-7)+100 mM Inosine 5′-PO4+1M Choline-Cl+800 mM L-Carnitine+5 mM Na-Phosphate (pH 8)+10 mM Na-Acetate (pH 7)
8. 100 mM 4-Hydroxyhippuric Acid (pH 5-7)+200 mM Uridine 5′-PO4+100 mM D-Fructose-1,6-Bisphosphate+100 mM N-Acetyl-L-Aspartate (pH 5-7)+100 mM L-Ornithine+800 mM Ectoine+5 mM Na-Phosphate (pH 8)+10 mM Na-Acetate (pH 7)
9. 200 mM Uridine 5′-PO4+100 mM Na-Glutamate (pH 5-7)+1M Sorbitol+200 mM Quinolinic Acid+5 mM Na-Phosphate (pH 8)+10 mM Na-Acetate (pH 7)
10. −80° C.

Following dilution of each sample in each respective RNA storage environment, samples were stored in a thermal cycler at 60° C. for about 24 hours and then analyzed by denaturing agarose gel electrophoresis as shown in FIGS. 74 C and D.

Orotic acid was adjusted to (pH 5-7) with NaOH and added to the composition as a suspension of orotate in water at the specified concentration. Prior to analysis by agarose gel electrophoresis, samples were diluted 2× with water and sample 6 with orotic acid was centrifuged to remove residual undissolved material to help improve analysis by gel electrophoresis. The agarose gel was also imaged at about 470-490 nm to help improve sample analysis.

### Example 127

### Accelerated RNA Stability Testing at 60° C.

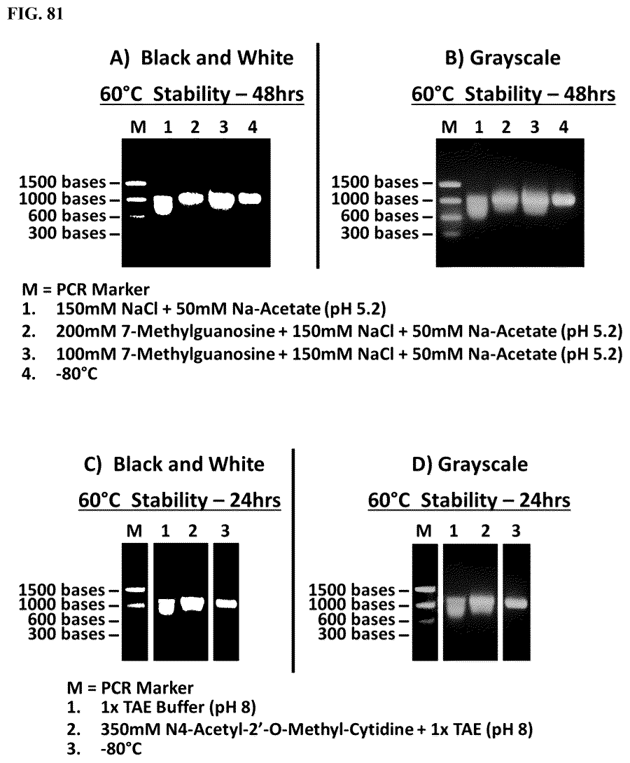

In vitro transcribed RNA was diluted at a ratio of about 1:10 (about 100-300 μg/mL) in different compositions containing 150 mM NaCl+50 mM Na-Acetate (pH 5.2) or one or more compounds in compositions with 150 mM NaCl+50 mM Na-Acetate (pH 5.2) as shown in FIGS. 81 A and B.
M=PCR Marker
1. 150 mM NaCl+50 mM Na-Acetate (pH 5.2)
2. 200 mM 7-Methylguanosine+150 mM NaCl+50 mM Na-Acetate (pH 5.2)
3. 100 mM 7-Methylguanosine+150 mM NaCl+50 mM Na-Acetate (pH 5.2)
4. −80° C.

Following dilution of each sample in each respective RNA storage environment, samples were stored in a thermal cycler at 60° C. for about 48 hours and then analyzed by denaturing agarose gel electrophoresis as shown in FIGS. 81A and B.

Prior to analysis by agarose gel electrophoresis, samples were diluted 2× with water, adjusted to pH 7 with 1M Tris-HCl (pH 7), and 7-Methylguanosine samples were centrifuged to remove residual undissolved material to help improve analysis by gel electrophoresis.

The following substances in one or more compositions were purchased as follows: 7-Methylguanosine (Cayman Chemical, Product #15988).

### Example 128

### Accelerated RNA Stability Testing at 60° C.

In vitro transcribed RNA was diluted at a ratio of about 1:10 (about 100-300 μg/mL) in different compositions containing 1×TAE Buffer (pH 8) or one or more compounds in compositions with 1×TAE Buffer (pH 8) as shown in FIGS. 81 C and D.
M=PCR Marker
1. 1×TAE Buffer (pH 8)
2. 350 mM N4-Acetyl-2′-O-Methyl-Cytidine+1×TAE (pH 8)
3. −80° C.

Following dilution of each sample in each respective RNA storage environment, samples were stored in a thermal cycler at 60° C. for about 24 hours and then analyzed by denaturing agarose gel electrophoresis as shown in FIGS. 81 C and D.

The following substances in one or more compositions were purchased as follows: N4-Acetyl-2′-O-Methyl-Cytidine (Cayman Chemical, Product #40285).

### Example 129

### Accelerated RNA Stability Testing at 50° C.

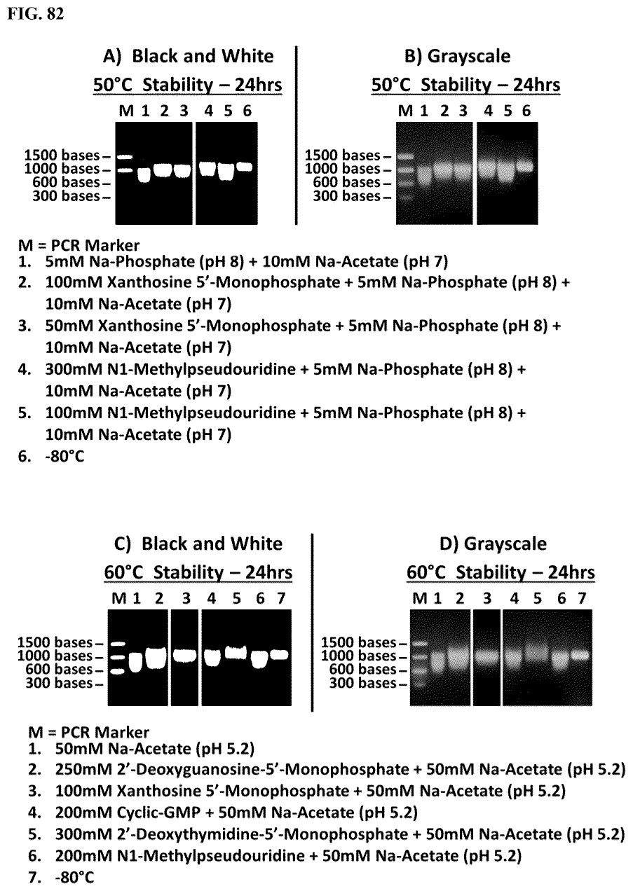

In vitro transcribed RNA was diluted at a ratio of about 1:10 (about 100-300 μg/mL) in different compositions containing 5 mM Na-Phosphate (pH 8)+10 mM Na-Acetate (pH 7) or one or more compounds in compositions with 5 mM Na-Phosphate (pH 8)+10 mM Na-Acetate (pH 7) as shown in FIGS. 82A and B.
M=PCR Marker
1. 5 mM Na-Phosphate (pH 8)+10 mM Na-Acetate (pH 7)
2. 100 mM Xanthosine 5′-Monophosphate+5 mM Na-Phosphate (pH 8)+10 mM Na-Acetate (pH 7)
3. 50 mM Xanthosine 5′-Monophosphate+5 mM Na-Phosphate (pH 8)+10 mM Na-Acetate (pH 7)
4. 300 mM N1-Methylpseudouridine+5 mM Na-Phosphate (pH 8)+10 mM Na-Acetate (pH 7)
5. 100 mM N1-Methylpseudouridine+5 mM Na-Phosphate (pH 8)+10 mM Na-Acetate (pH 7)
6. −80° C.

Following dilution of each sample in each respective RNA storage environment, samples were stored in a thermal cycler at 50° C. for about 24 hours and then analyzed by denaturing agarose gel electrophoresis as shown in FIGS. 82A and B.

The following substances in one or more compositions were purchased as follows: Xanthosine 5′-Monophosphate (Cayman Chemical, Product #18134), N1-Methylpseudouridine (Cayman Chemical, Product #35401).

### Example 130

### Accelerated RNA Stability Testing at 60° C.

In vitro transcribed RNA was diluted at a ratio of about 1:10 (about 100-300 μg/mL) in different compositions containing 50 mM Na-Acetate (pH 5.2) or one or more compounds in compositions with 50 mM Na-Acetate (pH 5.2) as shown in FIGS. 82 C and D.
M=PCR Marker
1. 50 mM Na-Acetate (pH 5.2)
2. 250 mM 2′-Deoxyguanosine-5′-Monophosphate+50 mM Na-Acetate (pH 5.2)
3. 100 mM Xanthosine 5′-Monophosphate+50 mM Na-Acetate (pH 5.2)
4. 200 mM Cyclic-GMP+50 mM Na-Acetate (pH 5.2)
5. 300 mM 2′-Deoxythymidine-5′-Monophosphate+50 mM Na-Acetate (pH 5.2)
6. 200 mM N1-Methylpseudouridine+50 mM Na-Acetate (pH 5.2)
7. −80° C.

Following dilution of each sample in each respective RNA storage environment, samples were stored in a thermal cycler at 60° C. for about 24 hours and then analyzed by denaturing agarose gel electrophoresis as shown in FIGS. 82 C and D.

Prior to analysis by agarose gel electrophoresis, samples were diluted 2× with water and adjusted to pH 7 with 1M Tris-HCl (pH 7) to help improve analysis by gel electrophoresis.

The following substances in one or more compositions were purchased as follows: 2′-Deoxyguanosine-5′-Monophosphate (Cayman Chemical, Product #16408), Cyclic Guanosine-Monophosphate (Cyclic-GMP) (Cayman Chemical, Product #18821), 2′-Deoxythymidine-5′-Monophosphate (Cayman Chemical, Product #33549).

### Example 131

### Accelerated RNA Stability Testing at 50° C.

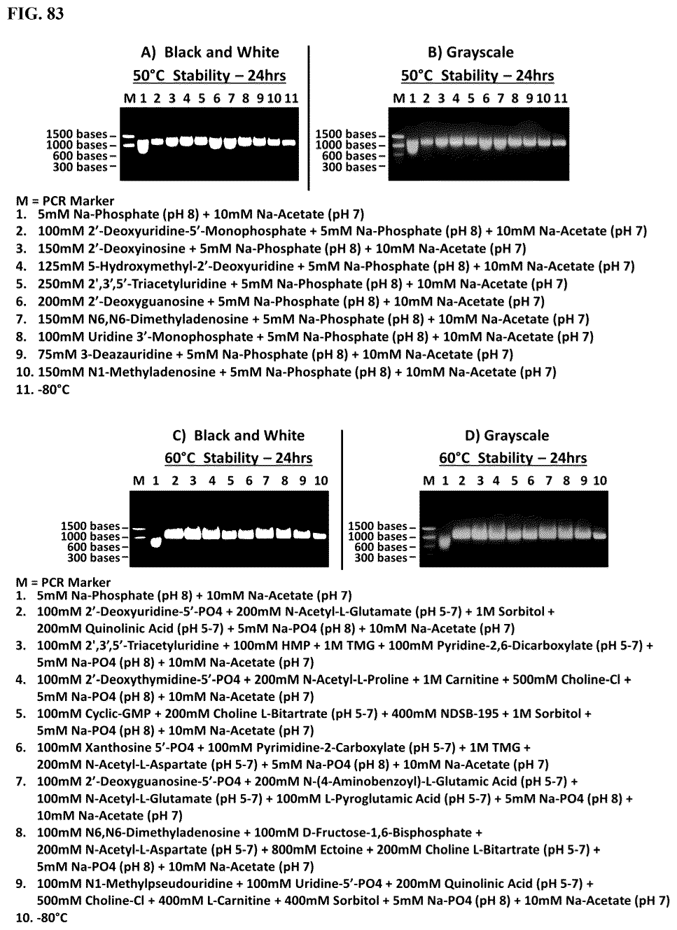

In vitro transcribed RNA was diluted at a ratio of about 1:10 (about 100-300 μg/mL) in different compositions containing 5 mM Na-Phosphate (pH 8)+10 mM Na-Acetate (pH 7) or one or more compounds in compositions with 5 mM Na-Phosphate (pH 8)+10 mM Na-Acetate (pH 7) as shown in FIGS. 83A and B.
M=PCR Marker
2. 100 mM 2′-Deoxyuridine-5′-Monophosphate+5 mM Na-Phosphate (pH 8)+10 mM Na-Acetate (pH 7)
3. 150 mM 2′-Deoxyinosine+5 mM Na-Phosphate (pH 8)+10 mM Na-Acetate (pH 7)
4. 125 mM 5-Hydroxymethyl-2′-Deoxyuridine+5 mM Na-Phosphate (pH 8)+10 mM Na-Acetate (pH 7)
5. 250 mM 2′,3′,5′-Triacetyluridine+5 mM Na-Phosphate (pH 8)+10 mM Na-Acetate (pH 7)
6. 200 mM 2′-Deoxyguanosine+5 mM Na-Phosphate (pH 8)+10 mM Na-Acetate (pH 7)
7. 150 mM N6,N6-Dimethyladenosine+5 mM Na-Phosphate (pH 8)+10 mM Na-Acetate (pH 7)
8. 100 mM Uridine 3′-Monophosphate+5 mM Na-Phosphate (pH 8)+10 mM Na-Acetate (pH 7)
9. 75 mM 3-Deazauridine+5 mM Na-Phosphate (pH 8)+10 mM Na-Acetate (pH 7)
10. 150 mM N1-Methyladenosine+5 mM Na-Phosphate (pH 8)+10 mM Na-Acetate (pH 7)
11. −80° C.

Following dilution of each sample in each respective RNA storage environment, samples were stored in a thermal cycler at 50° C. for about 24 hours and then analyzed by denaturing agarose gel electrophoresis as shown in FIGS. 83A and B.
2′-Deoxyuridine-5′-Monophosphate was heated and sonicated prior to being added to the composition to help improve solubility.

Prior to analysis by agarose gel electrophoresis, samples were diluted 2× with water, adjusted to pH 7 with 1M Tris-HCl (pH 7), and centrifuged to remove residual undissolved material to help improve analysis by gel electrophoresis.

The following substances in one or more compositions were purchased as follows: 2′-Deoxyuridine-5′-Monophosphate (Cayman Chemical, Product #34314), 2′-Deoxyinosine (Cayman Chemical, Product #31507), 5-Hydroxymethyl-2′-Deoxyuridine (Cayman Chemical, Product #23381), 2′,3′,5′-Triacetyluridine (Cayman Chemical, Product #27445), 2′-Deoxyguanosine (Cayman Chemical, Product #9002864), N6,N6-Dimethyladenosine (Cayman Chemical, Product #35348), Uridine 3′-Monophosphate (Cayman Chemical, Product #16774), 3-Deazauridine (Cayman Chemical, Product #23125), N1-Methyladenosine (Cayman Chemical, Product #16937).

### Example 132

### Accelerated RNA Stability Testing at 60° C.

In vitro transcribed RNA was diluted at a ratio of about 1:10 (about 100-300 μg/mL) in different compositions containing 5 mM Na-Phosphate (pH 8)+10 mM Na-Acetate (pH 7) or one or more compounds in compositions with 5 mM Na-Phosphate (pH 8)+10 mM Na-Acetate (pH 7) as shown in FIGS. 83 C and D.
M=PCR Marker
1. 5 mM Na-Phosphate (pH 8)+10 mM Na-Acetate (pH 7)
2. 100 mM 2′-Deoxyuridine-5′-PO4+200 mM N-Acetyl-L-Glutamate (pH 5-7)+1M Sorbitol+200 mM Quinolinic Acid (pH 5-7)+5 mM Na—PO4 (pH 8)+10 mM Na-Acetate (pH 7)
3. 100 mM 2′,3′,5′-Triacetyluridine+100 mM HMP+1M TMG+100 mM Pyridine-2,6-Dicarboxylate (pH 5-7)+5 mM Na—PO4 (pH 8)+10 mM Na-Acetate (pH 7)
4. 100 mM 2′-Deoxythymidine-5′-PO4+200 mM N-Acetyl-L-Proline+1M L-Carnitine+500 mM Choline-Cl+5 mM Na—PO4 (pH 8)+10 mM Na-Acetate (pH 7)
5. 100 mM Cyclic-GMP+200 mM Choline L-Bitartrate (pH 5-7)+400 mM NDSB-195+1M Sorbitol+5 mM Na—PO4 (pH 8)+10 mM Na-Acetate (pH 7)
6. 100 mM Xanthosine 5′-PO4+100 mM Pyrimidine-2-Carboxylate (pH 5-7)+1M TMG+200 mM N-Acetyl-L-Aspartate (pH 5-7)+5 mM Na—PO4 (pH 8)+10 mM Na-Acetate (pH 7)
7. 100 mM 2′-Deoxyguanosine-5′-PO4+200 mM N-(4-Aminobenzoyl)-L-Glutamic Acid (pH 5-7)+100 mM N-Acetyl-L-Glutamate (pH 5-7)+100 mM L-Pyroglutamic Acid (pH 5-7)+5 mM Na—PO4 (pH 8)+10 mM Na-Acetate (pH 7)
8. 100 mM N6,N6-Dimethyladenosine+100 mM D-Fructose-1,6-Bisphosphate+200 mM N-Acetyl-L-Aspartate (pH 5-7)+800 mM Ectoine+200 mM Choline L-Bitartrate (pH 5-7)+5 mM Na—PO4 (pH 8)+10 mM Na-Acetate (pH 7)
9. 100 mM N1-Methylpseudouridine+100 mM Uridine-5′-PO4+200 mM Quinolinic Acid (pH 5-7)+500 mM Choline-Cl+400 mM L-Carnitine+400 mM Sorbitol+5 mM Na—PO4 (pH 8)+10 mM Na-Acetate (pH 7)
10. −80° C.

Following dilution of each sample in each respective RNA storage environment, samples were stored in a thermal cycler at 60° C. for about 24 hours and then analyzed by denaturing agarose gel electrophoresis as shown in FIGS. 83 C and D.

2′-Deoxyuridine-5′-Monophosphate was heated and sonicated prior to being added to the composition to help improve solubility.

Prior to analysis by agarose gel electrophoresis, samples were diluted 2× with water, adjusted to pH 7 with 1M Tris-HCl (pH 7), and centrifuged to remove residual undissolved material to help improve analysis by gel electrophoresis.

The following substances in one or more compositions were purchased as follows: Choline L-Bitartrate (Liftmode, operated by Synaptent, LLC)

### Example 133-A

Synthesis of Additional Modified Carbohydrates and Polysaccharides with Selected Phosphate or Cationic Quaternary Ammonium Ratios

Additional modified carbohydrates were synthesized and used for RNA stability testing in Examples 133-B, 134, 135, and 136 below.

### Synthesis of Additional Phosphate Modified Carbohydrates

Protocol 1 in Example 1 above was used to produce variations of phosphate modified carbohydrates with selected ratios of sodium phosphate to carbohydrate as listed in Table 8-1.

Briefly, 0.2 g carbohydrate was mixed with 0.05-0.2 g sodium phosphate (pH 5-6) dissolved in 150 μL water to create a paste. The mixed paste was then incubated at about 280-300° F. for about 20-60 min depending on the carbohydrate. Carbohydrates were removed at the first sign of light brown discoloration, if any occurred. During heating, water was allowed to evaporate and the samples were reacted dry to semi-dry. After the reaction, each sample was resuspended in 200-500 μL molecular grade water, depending on viscosity, and dialyzed against about 100 mL of water for about 48-72 hrs at room temperature (about 20-30° C.). Starch samples were dialyzed using a 2,000 Da molecular weight cutoff membrane and sorbitol samples were dialyzed using a 200 Da molecular weight cutoff membrane. Following dialysis, samples were dried down, and the dry weight was measured on an analytical balance. The dried material was then resuspended in molecular grade water at the desired working concentration and the pH was adjusted to about 7 using either NaOH or HCl.

The type of carbohydrate used and selected time, temperature, and reactant variations are listed in Table 8-1.

TABLE 8-1
Phosphate Modified Carbohydrates Produced Using Protocol 1

| Carbohydrate | Reactant | Heating | Heating | | |
|---|---|---|---|---|---|
| (Carb) | Quantity/Ratios | Temperature | Time | | Reference Name |
| Starch | 0.2 g Carb + | 280-300° F. | 20 | min | Na—PO4 Starch |
| | 0.05 g Na—PO4 | | | | 1:4 Ratio |
| Starch | 0.2 g Carb + | 280-300° F. | 20 | min | Na—PO4 Starch |
| | 0.1 g Na—PO4 | | | | 1:2 Ratio |
| Starch | 0.2 g Carb + | 280-300° F. | 20 | min | Na—PO4 Starch |
| | 0.2 g Na—PO4 | | | | 1:1 Ratio |
| Zulkowsky Starch | 0.2 g Carb + | 280-300° F. | 20 | min | Na—PO4 Zulkowsky |
| | 0.05 g Na—PO4 | | | | Starch 1:4 Ratio |
| Sorbitol | 0.2 g Carb + | 280-300° F. | 1 | hr | Na—PO4 Sorbitol |
| | 0.05 g Na—PO4 | | | | 1:4 Ratio |
| Sorbitol | 0.2 g Carb + | 280-300° F. | 1 | hr | Na—PO4 Sorbitol |
| | 0.1 g Na—PO4 | | | | 1:2 Ratio |
| Sorbitol | 0.2 g Carb + | 280-300° F. | 1 | hr | Na—PO4 Sorbitol |
| | 0.2 g Na—PO4 | | | | 1:1 Ratio |

### Synthesis of Additional Cationic Quaternary Ammonium Modified Carbohydrates

Protocol 2 in Example 2 above was used to produce variations of cationic quaternary ammonium modified carbohydrates with selected ratios of betaine-HCl to carbohydrate as listed in Table 8-2.

Briefly, 0.2 g carbohydrate was mixed with 0.05-0.2 g betaine-HCl dissolved in 200 μL water supplemented with 1 μL 85% lactic acid solution to create a paste. The mixed paste was then incubated at about 200-220° F. for about 30-60 min depending on the carbohydrate. Carbohydrates were removed at the first sign of light brown discoloration, if any occurred. During heating, water was allowed to evaporate and the samples were reacted dry to semi-dry. After the reaction, each sample was resuspended in 200-500 μL molecular grade water, depending on viscosity, and the pH of the samples was adjusted to about 7 using NaOH. The samples were then dialyzed against about 100 mL of water for about 48-72 hrs at room temperature (about 20-30° C.). Starch samples were dialyzed using a 2,000 Da molecular weight cutoff membrane and sorbitol samples were dialyzed using a 200 Da molecular weight cutoff membrane. Following dialysis, samples were dried down, and the dry weight was measured on an analytical balance. The dried material was then resuspended in molecular grade water at the desired working concentration and the pH was adjusted to about 7 using either NaOH or HCl.

The type of carbohydrate used and selected, time, temperature, and reactant variations are listed in Table 8-2.

TABLE 8-2
Cationic Quaternary Ammonium Modified
Carbohydrates Produced Using Protocol 2

| Carbohydrate | Reactant | Heating | Heating | | |
|---|---|---|---|---|---|
| (Carb) | Quantity/Ratios | Temperature | Time | | Reference Name |
| Starch | 0.2 g Carb + | 200-220° F. | 30 | min | Cationic-Starch |
| | 0.05 g Betaine-HCl | | | | 1:4 Ratio |
| | 1 uL Lactic Acid | | | | |
| Starch | 0.2 g Carb + | 200-220° F. | 30 | min | Cationic-Starch |
| | 0.1 g Betaine-HCl | | | | 1:2 Ratio |
| | 1 uL Lactic Acid | | | | |
| Starch | 0.2 g Carb + | 200-220° F. | 30 | min | Cationic-Starch |
| | 0.2 g Betaine-HCl | | | | 1:1 Ratio |
| | 1 uL Lactic Acid | | | | |
| Zulkowsky Starch | 0.2 g Carb + | 200-220° F. | 30 | min | Cationic-Zulkowsky |
| | 0.05 g Betaine-HCl | | | | Starch 1:4 Ratio |
| | 1 uL Lactic Acid | | | | |
| Sorbitol | 0.2 g Carb + | 200-220° F. | 1 | hr | Cationic-Sorbitol |
| | 0.05 g Betaine-HCl | | | | 1:4 Ratio |
| | 1 uL Lactic Acid | | | | |
| Sorbitol | 0.2 g Carb + | 200-220° F. | 1 | hr | Cationic-Sorbitol |
| | 0.1 g Betaine-HCl | | | | 1:2 Ratio |
| | 1 uL Lactic Acid | | | | |
| Sorbitol | 0.2 g Carb + | 200-220° F. | 1 | hr | Cationic-Sorbitol |
| | 0.2 g Betaine-HCl | | | | 1:1 Ratio |
| | 1 uL Lactic Acid | | | | |

### Synthesis of Additional Dual Phosphate and Cationic Quaternary Ammonium Modified Carbohydrates

Protocol 4 in Example 4 above was used to produce variations of dual phosphate and cationic quaternary ammonium modified carbohydrates with selected ratios of phosphate modified carbohydrate to betaine-HCl as listed in Table 8-3.

Briefly, 0.1 g phosphate modified carbohydrate was mixed with 0.0125-0.05 g betaine-HCl dissolved in 100 μL water supplemented with 1 μL 85% lactic acid solution to create a paste. The mixed paste was then incubated at about 200-220° F. for about 30 min depending on the carbohydrate. Carbohydrates were removed at the first sign of light brown discoloration, if any occurred. During heating, water was allowed to evaporate and the samples were reacted dry to semi-dry. After the reaction, each sample was resuspended in 200-500 μL molecular grade water, depending on viscosity, and the pH of the samples was adjusted to about 7 using NaOH. The samples were then dialyzed against about 100 mL of water for about 48-72 hrs at room temperature (about 20-30° C.). Starch samples were dialyzed using a 2,000 Da molecular weight cutoff membrane and sorbitol samples were dialyzed using a 200 Da molecular weight cutoff membrane. Following dialysis, samples were dried down, and the dry weight was measured on an analytical balance. The dried material was then resuspended in molecular grade water at the desired working concentration and the pH was adjusted to about 7 using either NaOH or HCl.

The type of carbohydrate used and selected, time, temperature, and reactant variations are listed in Table 8-3.

TABLE 8-3
Dual Phosphate and Cationic Quaternary Ammonium
Modified Carbohydrates Produced Using Protocol 4

| Carbohydrate | Reactant | Heating | Heating | |
|---|---|---|---|---|
| (Carb) | Quantity/Ratios | Temperature | Time | Reference Name |
| Na—PO4 Starch | 0.1 g Carb + | 200-220° F. | 30 min | Dual Cationic-PO4 |
| 1:4 Ratio | 0.05 g Betaine-HCl | | | Starch 2x Cationic |
| | 1 uL Lactic Acid | | | (Cat1:2/1:4PO4) |
| Na—PO4 Starch | 0.1 g Carb + | 200-220° F. | 30 min | Dual Equal Ratio |
| 1:4 Ratio | 0.025 g Betaine-HCl | | | Cationic-PO4 Starch |
| | 1 uL Lactic Acid | | | (Cat1:4/1:4PO4) |
| Na—PO4 Starch | 0.1 g Carb + | 200-220° F. | 30 min | Dual PO4-Cationic |
| 1:4 Ratio | 0.0125 g Betaine-HCl | | | Starch 2x PO4 |
| | 1 uL Lactic Acid | | | (Cat1:8/1:4PO4) |
| Na—PO4 Starch | 0.1 g Carb + | 200-220° F. | 30 min | Dual PO4-Cationic |
| 1:2 Ratio | 0.025 g Betaine-HCl | | | Starch 2x PO4 |
| | 1 uL Lactic Acid | | | (Cat1:4/1:2PO4) |

### Synthesis of Additional Crosslinked Phosphate or Cationic Quaternary Ammonium Modified Carbohydrates

Protocol 5 in Example 5-B above was used to produce variations of crosslinked modified carbohydrates using citric acid and selected modified carbohydrates as listed in Table 8-4.

Briefly, 0.1 g of modified carbohydrate was mixed with 1.5-15 mg citric acid dissolved in 100 μL water to create a paste. The mixed paste was then incubated at about 280-300° F. for about 5-30 min depending on the carbohydrate. Carbohydrates were removed at the first sign of light brown discoloration, if any occurred. During heating, water was allowed to evaporate and the samples were reacted dry to semi-dry. After the reaction, each sample was resuspended in 200 μL-1 mL molecular grade water, depending on viscosity, and the pH of the samples was adjusted to about 7 using NaOH. The samples were then dialyzed against about 100 ml of water for about 48-72 hrs at room temperature (about 20-30° C.). Starch samples were dialyzed using a 2,000 Da molecular weight cutoff membrane and sorbitol samples were dialyzed using a 500 Da molecular weight cutoff membrane. Following dialysis, samples were dried down, and the dry weight was measured on an analytical balance. The dried material was then resuspended in molecular grade water at the desired working concentration and the pH was adjusted to about 7 using either NaOH or HCl.

In some cases, both a phosphate modified carbohydrate and a cationic quaternary ammonium modified carbohydrate were crosslinked together as listed in Table 8-4. The type of carbohydrate used and selected, time, temperature, and reactant variations are listed in Table 8-4

TABLE 8-4
Crosslinked Modified Carbohydrates Produced Using Protocol 5

| Carbohydrate | Reactant | Heating | Heating | | |
|---|---|---|---|---|---|
| (Carb) | Quantity/Ratios | Temperature | Time | | Reference Name |
| Na—PO4 Starch 1:4 | 0.1 g Carb + | 280-300° F. | 5-10 | min | Citric X-Link Na—PO4 |
| Ratio | 1.5 mg Citric Acid | | | | Starch 1:4 Ratio |
| Na—PO4 Starch 1:2 | 0.1 g Carb + | 280-300° F. | 5-10 | min | Citric X-Link Na—PO4 |
| Ratio | 1.5 mg Citric Acid | | | | Starch 1:2 Ratio |
| Na—PO4 Starch 1:1 | 0.1 g Carb + | 280-300° F. | 5-10 | min | Citric X-Link Na—PO4 |
| Ratio | 1.5 mg Citric Acid | | | | Starch 1:1 Ratio |
| Na—PO4 Zulkowsky | 0.1 g Carb + | 280-300° F. | 5-10 | min | Citric X-Link Na—PO4 |
| Starch 1:4 Ratio | 1.5 mg Citric Acid | | | | Zulkowsky |
| | | | | | Starch 1:4 Ratio |
| Cationic-Starch 1:4 | 0.1 g Carb + | 280-300° F. | 5-10 | min | Citric X-Link |
| Ratio | 1.5 mg Citric Acid | | | | Cationic Starch 1:4 |
| | | | | | Ratio |
| Cationic-Starch 1:2 | 0.1 g Carb + | 280-300° F. | 5-10 | min | Citric X-Link |
| Ratio | 1.5 mg Citric Acid | | | | Cationic Starch 1:2 |
| | | | | | Ratio |
| Cationic-Starch 1:1 | 0.1 g Carb + | 280-300° F. | 5-10 | min | Citric X-Link |
| Ratio | 1.5 mg Citric Acid | | | | Cationic Starch 1:1 |
| | | | | | Ratio |
| Cationic-Zulkowsky | 0.1 g Carb + | 280-300° F. | 5-10 | min | Citric X-Link |
| Starch 1:4 Ratio | 1.5 mg Citric Acid | | | | Cationic Zulkowsky |
| | | | | | Starch 1:4 Ratio |
| Na—PO4 Starch 1:4 | 0.05 g Each Carb + | 280-300° F. | 5-10 | min | Citric X-Link Combo |
| Ratio + Cationic- | 1.5 mg Citric Acid | | | | Cationic-PO4 Starch |
| Starch 1:4 Ratio | | | | | Equal Ratio 1:4 |
| Na—PO4 Starch 1:2 | 0.05 g Each Carb + | 280-300° F. | 5-10 | min | Citric X-Link Combo |
| Ratio + Cationic- | 1.5 mg Citric Acid | | | | Cationic-PO4 Starch |
| Starch 1:2 Ratio | | | | | Equal Ratio 1:2 |
| Na—PO4 Starch 1:1 | 0.05 g Each Carb + | 280-300° F. | 5-10 | min | Citric X-Link Combo |
| Ratio + Cationic- | 1.5 mg Citric Acid | | | | Cationic-PO4 Starch |
| Starch 1:1 Ratio | | | | | Equal Ratio 1:1 |
| Na—PO4 Zulkowsky | 0.05 g Each Carb + | 280-300° F. | 5-10 | min | Citric X-Link Combo |
| Starch 1:4 Ratio + | 1.5 mg Citric Acid | | | | Cationic-PO4 |
| Cationic-Zulkowsky | | | | | Zulkowsky Starch |
| Starch 1:4 Ratio | | | | | Equal Ratio 1:4 |
| Na—PO4 Sorbitol 1:4 | 0.1 g Carb + | 280-300° F. | 30 | min | Citric X-Link Na—PO4 |
| Ratio | 15 mg Citric Acid | | | | Sorbitol 1:4 Ratio |
| Na—PO4 Sorbitol 1:2 | 0.1 g Carb + | 280-300° F. | 30 | min | Citric X-Link Na—PO4 |
| Ratio | 15 mg Citric Acid | | | | Sorbitol 1:2 Ratio |
| Na—PO4 Sorbitol 1:1 | 0.1 g Carb + | 280-300° F. | 30 | min | Citric X-Link Na—PO4 |
| Ratio | 15 mg Citric Acid | | | | Sorbitol 1:1 Ratio |
| Cationic-Sorbitol | 0.1 g Carb + | 280-300° F. | 30 | min | Citric X-Link |
| 1:4 Ratio | 15 mg Citric Acid | | | | Cationic-Sorbitol 1:4 |
| | | | | | Ratio |
| Cationic-Sorbitol | 0.1 g Carb + | 280-300° F. | 30 | min | Citric X-Link |
| 1:2 Ratio | 15 mg Citric Acid | | | | Cationic-Sorbitol 1:2 |
| | | | | | Ratio |
| Cationic-Sorbitol | 0.1 g Carb + | 280-300° F. | 30 | min | Citric X-Link |
| 1:1 Ratio | 15 mg Citric Acid | | | | Cationic-Sorbitol 1:1 |
| | | | | | Ratio |
| Na—PO4 Sorbitol 1:4 | 0.05 g Each Carb + | 280-300° F. | 30 | min | Citric X-Link Combo |
| Ratio + Cationic- | 15 mg Citric Acid | | | | Sorbitol Equal Ratio |
| Sorbitol 1:4 Ratio | | | | | 1:4 |
| Na—PO4 Sorbitol 1:2 | 0.05 g Each Carb + | 280-300° F. | 30 | min | Citric X-Link Combo |
| Ratio + Cationic- | 15 mg Citric Acid | | | | Sorbitol Equal Ratio |
| Sorbitol 1:2 Ratio | | | | | 1:2 |
| Na—PO4 Sorbitol 1:1 | 0.05 g Each Carb + | 280-300° F. | 30 | min | Citric X-Link Combo |
| Ratio + Cationic- | 15 mg Citric Acid | | | | Sorbitol Equal Ratio |
| Sorbitol 1:1 Ratio | | | | | 1:1 |
| Na—PO4 Sorbitol 1:1 | 0.05 g Each Carb + | 280-300° F. | 30 | min | Citric X-Link Combo |
| Ratio + Cationic- | 15 mg Citric Acid | | | | 2x PO4 Sorbitol |
| Sorbitol 1:2 Ratio | | | | | 1:1/1:2 Ratio |
| Na—PO4 Sorbitol 1:2 | 0.05 g Each Carb + | 280-300° F. | 30 | min | Citric X-Link Combo |
| Ratio + Cationic- | 15 mg Citric Acid | | | | 2x Cationic Sorbitol |
| Sorbitol 1:1 Ratio | | | | | 1:1/1:2 Ratio |

### Example 133-B

The modified carbohydrates described above in example 133-A were used for RNA stability testing described as follows.

### Accelerated RNA Stability Testing at 60° C.

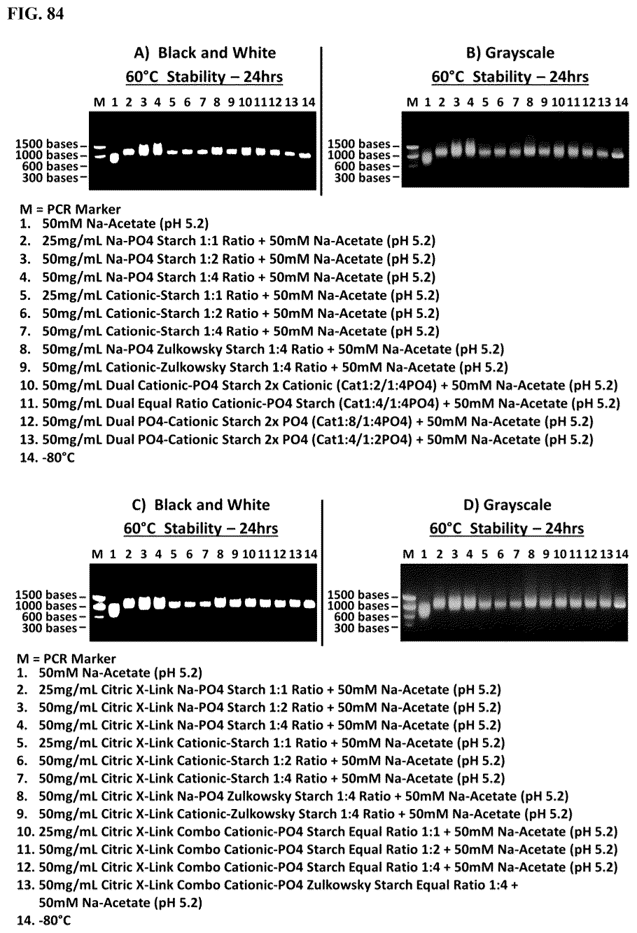

In vitro transcribed RNA was diluted at a ratio of about 1:10 (about 100-300 μg/mL) in different compositions containing 50 mM Na-Acetate (pH 5.2) or one or more compounds in compositions with 50 mM Na-Acetate (pH 5.2) as shown in FIGS. 84A and B.
M=PCR Marker
1. 50 mM Na-Acetate (pH 5.2)
2. 25 mg/mL Na—PO4 Starch 1:1 Ratio+50 mM Na-Acetate (pH 5.2)
3. 50 mg/mL Na—PO4 Starch 1:2 Ratio+50 mM Na-Acetate (pH 5.2)
4. 50 mg/mL Na—PO4 Starch 1:4 Ratio+50 mM Na-Acetate (pH 5.2)
5. 25 mg/mL Cationic-Starch 1:1 Ratio+50 mM Na-Acetate (pH 5.2)
6. 50 mg/mL Cationic-Starch 1:2 Ratio+50 mM Na-Acetate (pH 5.2)
7. 50 mg/mL Cationic-Starch 1:4 Ratio+50 mM Na-Acetate (pH 5.2)
8. 50 mg/mL Na—PO4 Zulkowsky Starch 1:4 Ratio+50 mM Na-Acetate (pH 5.2)
9. 50 mg/mL Cationic-Zulkowsky Starch 1:4 Ratio+50 mM Na-Acetate (pH 5.2)
10. 50 mg/mL Dual Cationic-PO4 Starch 2× Cationic (Cat1:2/1:4PO4)+50 mM Na-Acetate (pH 5.2)
11. 50 mg/mL Dual Equal Ratio Cationic-PO4 Starch (Cat1:4/1:4PO4)+50 mM Na-Acetate (pH 5.2)
12. 50 mg/mL Dual PO4-Cationic Starch 2× PO₄ (Cat1:8/1:4PO4)+50 mM Na-Acetate (pH 5.2)
13. 50 mg/mL Dual PO4-Cationic Starch 2× PO₄ (Cat1: 4/1: 2PO4)+50 mM Na-Acetate (pH 5.2)
14. −80° C.

Following dilution of each sample in each respective RNA storage environment, samples were stored in a thermal cycler at 60° C. for about 24 hours and then analyzed by denaturing agarose gel electrophoresis as shown in FIGS. 84A and B.

Prior to analysis by agarose gel electrophoresis, samples were incubated with 2.5 mM PAA ˜8 kDa for 1 hr at room temperature to help improve analysis by agarose gel electrophoresis.

### Example 134

The modified carbohydrates described above in example 133-A were used for RNA stability testing described as follows.

### Accelerated RNA Stability Testing at 60° C.

In vitro transcribed RNA was diluted at a ratio of about 1:10 (about 100-300 μg/mL) in different compositions containing 50 mM Na-Acetate (pH 5.2) or one or more compounds in compositions with 50 mM Na-Acetate (pH 5.2) as shown in FIGS. 84 C and D.
M=PCR Marker
1. 50 mM Na-Acetate (pH 5.2)
2. 25 mg/mL Citric X-Link Na—PO4 Starch 1:1 Ratio+50 mM Na-Acetate (pH 5.2)
3. 50 mg/mL Citric X-Link Na—PO4 Starch 1:2 Ratio+50 mM Na-Acetate (pH 5.2)
4. 50 mg/mL Citric X-Link Na—PO4 Starch 1:4 Ratio+50 mM Na-Acetate (pH 5.2)
5. 25 mg/mL Citric X-Link Cationic-Starch 1:1 Ratio+50 mM Na-Acetate (pH 5.2)
6. 50 mg/mL Citric X-Link Cationic-Starch 1:2 Ratio+50 mM Na-Acetate (pH 5.2)
7. 50 mg/mL Citric X-Link Cationic-Starch 1:4 Ratio+50 mM Na-Acetate (pH 5.2)
8. 50 mg/mL Citric X-Link Na—PO4 Zulkowsky Starch 1:4 Ratio+50 mM Na-Acetate (pH 5.2)
9. 50 mg/mL Citric X-Link Cationic-Zulkowsky Starch 1:4 Ratio+50 mM Na-Acetate (pH 5.2)
10. 25 mg/mL Citric X-Link Combo Cationic-PO4 Starch Equal Ratio 1:1+50 mM Na-Acetate (pH 5.2)
11. 50 mg/mL Citric X-Link Combo Cationic-PO4 Starch Equal Ratio 1:2+50 mM Na-Acetate (pH 5.2)
12. 50 mg/mL Citric X-Link Combo Cationic-PO4 Starch Equal Ratio 1:4+50 mM Na-Acetate (pH 5.2)
13. 50 mg/mL Citric X-Link Combo Cationic-PO4 Zulkowsky Starch Equal Ratio 1:4+50 mM Na-Acetate (pH 5.2)
14. −80° C.

Following dilution of each sample in each respective RNA storage environment, samples were stored in a thermal cycler at 60° C. for about 24 hours and then analyzed by denaturing agarose gel electrophoresis as shown in FIGS. 84 C and D.

Prior to analysis by agarose gel electrophoresis, samples were incubated with 2.5 mM PAA ˜8 kDa for 1 hr at room temperature to help improve analysis by agarose gel electrophoresis.

### Example 135

The modified carbohydrates described above in example 133-A were used for RNA stability testing described as follows.

### Accelerated RNA Stability Testing at 60° C.

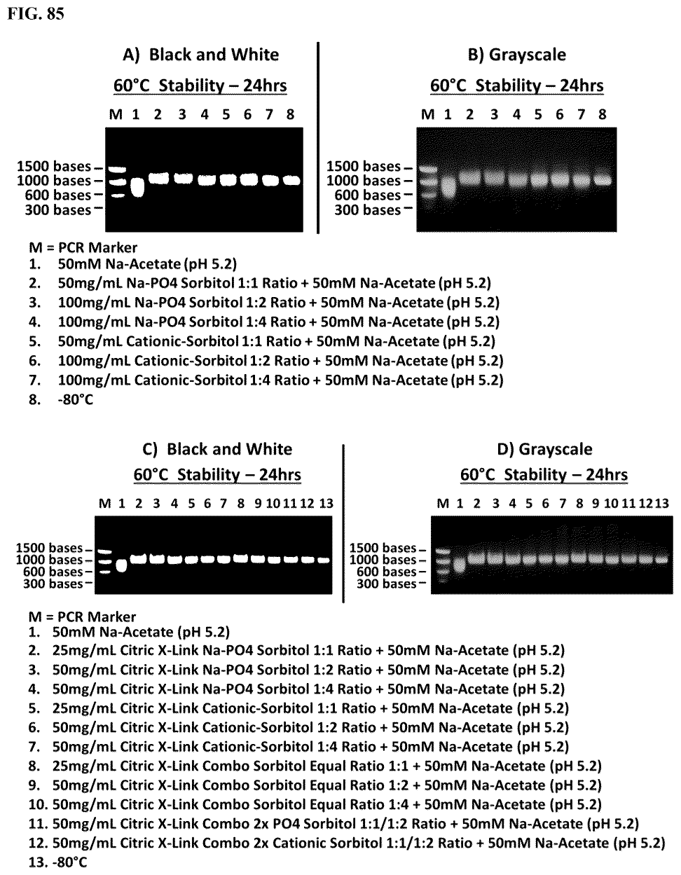

In vitro transcribed RNA was diluted at a ratio of about 1:10 (about 100-300 μg/mL) in different compositions containing 50 mM Na-Acetate (pH 5.2) or one or more compounds in compositions with 50 mM Na-Acetate (pH 5.2) as shown in FIGS. 85A and B.
M=PCR Marker
1. 50 mM Na-Acetate (pH 5.2)
2. 50 mg/mL Na—PO4 Sorbitol 1:1 Ratio+50 mM Na-Acetate (pH 5.2)
3. 100 mg/mL Na—PO4 Sorbitol 1:2 Ratio+50 mM Na-Acetate (pH 5.2)
4. 100 mg/mL Na—PO4 Sorbitol 1:4 Ratio+50 mM Na-Acetate (pH 5.2)
5. 50 mg/mL Cationic-Sorbitol 1:1 Ratio+50 mM Na-Acetate (pH 5.2)
6. 100 mg/mL Cationic-Sorbitol 1:2 Ratio+50 mM Na-Acetate (pH 5.2)
7. 100 mg/mL Cationic-Sorbitol 1:4 Ratio+50 mM Na-Acetate (pH 5.2)
8. −80° C.

Following dilution of each sample in each respective RNA storage environment, samples were stored in a thermal cycler at 60° C. for about 24 hours and then analyzed by denaturing agarose gel electrophoresis as shown in FIGS. 85A and B.

Prior to analysis by agarose gel electrophoresis, samples were incubated with 2.5 mM PAA ˜8 kDa for 1 hr at room temperature to help improve analysis by agarose gel electrophoresis.

### Example 136

The modified carbohydrates described above in example 133-A were used for RNA stability testing described as follows.

### Accelerated RNA Stability Testing at 60° C.

In vitro transcribed RNA was diluted at a ratio of about 1:10 (about 100-300 μg/mL) in different compositions containing 50 mM Na-Acetate (pH 5.2) or one or more compounds in compositions with 50 mM Na-Acetate (pH 5.2) as shown in FIGS. 85 C and D.
M=PCR Marker
1. 50 mM Na-Acetate (pH 5.2)
2. 25 mg/mL Citric X-Link Na—PO4 Sorbitol 1:1 Ratio+50 mM Na-Acetate (pH 5.2)
3. 50 mg/mL Citric X-Link Na—PO4 Sorbitol 1:2 Ratio+50 mM Na-Acetate (pH 5.2)
4. 50 mg/mL Citric X-Link Na—PO4 Sorbitol 1:4 Ratio+50 mM Na-Acetate (pH 5.2)
5. 25 mg/mL Citric X-Link Cationic-Sorbitol 1:1 Ratio+50 mM Na-Acetate (pH 5.2)
6. 50 mg/mL Citric X-Link Cationic-Sorbitol 1:2 Ratio+50 mM Na-Acetate (pH 5.2)
7. 50 mg/mL Citric X-Link Cationic-Sorbitol 1:4 Ratio+50 mM Na-Acetate (pH 5.2)
8. 25 mg/mL Citric X-Link Combo Sorbitol Equal Ratio 1:1+50 mM Na-Acetate (pH 5.2)
9. 50 mg/mL Citric X-Link Combo Sorbitol Equal Ratio 1:2+50 mM Na-Acetate (pH 5.2)
10. 50 mg/mL Citric X-Link Combo Sorbitol Equal Ratio 1:4+50 mM Na-Acetate (pH 5.2)
11. 50 mg/mL Citric X-Link Combo 2× PO4 Sorbitol 1:1/1:2 Ratio+50 mM Na-Acetate (pH 5.2)
12. 50 mg/mL Citric X-Link Combo 2× Cationic Sorbitol 1:1/1:2 Ratio+50 mM Na-Acetate (pH 5.2)
13. −80° C.

Following dilution of each sample in each respective RNA storage environment, samples were stored in a thermal cycler at 60° C. for about 24 hours and then analyzed by denaturing agarose gel electrophoresis as shown in FIGS. 85 C and D.

Prior to analysis by agarose gel electrophoresis, samples were incubated with 2.5 mM PAA ˜8 kDa for 1 hr at room temperature to help improve analysis by agarose gel electrophoresis.

### Example 137-A

### Multi-Nucleotide Synthesis for RNA Stability Testing

Synthesis of multi-nucleotides is known in the art and also commercially available. An example commercial vendor is Integrated DNA Technologies (Coralville, IA). The multi-nucleotides used in the following examples, were synthesized as follows.

Linear DNA sequences with a T7 RNA polymerase promoter followed by the sequence for desired multi-nucleotides were ordered from Twist Bioscience (South San Francisco, CA). HaeIII or DraI restriction endonuclease sites were included immediately downstream of the desired multi-nucleotide sequence to remove unwanted nucleotides following the T7 RNA polymerase promoter and the desired multi-nucleotide sequence. The specific endonuclease site was selected depending on the desired sequence. A general schematic of the sequence is as follows: DNA-[T7 promoter]-[Multi-Nucleotide]-[restriction site]-DNA

The linear DNA was restriction digested to remove downstream DNA using either HaeIII or DraI endonuclease (New England Biolabs, Product #R0108 and R0129) according to the manufacturer's instructions. Following restriction digestion, a DNA fragment of the correct size was confirmed by agarose gel electrophoresis. The resulting restriction digested DNA containing the T7 RNA polymerase promoter and desired downstream multi-nucleotide sequence was then gel purified using agarose gel electrophoresis by excising the band of interest and extracting the DNA. A general schematic of the restriction digested DNA sequence is as follows: DNA-[T7 promoter]-[Multi-Nucleotide]

The resulting gel purified DNA with T7 RNA polymerase promoter immediately followed by the multi-nucleotide sequence of interest was used to perform in vitro transcription using a T7 RNA polymerase and selected nucleotide triphosphates for each desired multi-nucleotide (New England Biolabs, Ipswich, MA; Product #M0251 and NO450). In vitro transcription to synthesize the multi-nucleotide of interest was carried out overnight (about 16-24 hrs) according to the manufacturer's directions, with the exception that unnecessary nucleotides not present in the multi-nucleotide of interest were not included in the reaction. Following in vitro transcription, the multi-nucleotides were analyzed by agarose gel electrophoresis and subsequently dialyzed for about 24 hrs against about 100 ml of water to remove residual mononucleotides. Following dialysis, the concentration of each multi-nucleotide was measured by absorbance at 260 nm using a Nanodrop ND-1000 (Thermo Fisher Scientific, Waltham, MA) and the samples were diluted to the desired working concentration for RNA stability testing.

Multi-Nucleotides used in RNA stability testing are listed in Table 9 below.

TABLE 9
Multi-Nucleotides Used in RNA Stability Testing

| | Reference | Restriction |
|---|---|---|
| Multi-Nucleotide | Name | Enzyme Used |
| GGGG | G4 | HaeIII |
| GAAAAGG | A4 | HaeIII |
| GGUUGGUUGG | G2U2 × 2 | HaeIII |
| GGUUUUGG | G2U4G2 | HaeIII |
| GUUUUUUUUUUUUGG | U12 | HaeIII |
| GGGUUGGGUUGGGUUGGG | G12U6 | HaeIII |
| GGGGGGGGGG | G10 | HaeIII |
| GUUUUUUGG | G1U6G2 | HaeIII |
| GUUUU | U4 | DraI |
| GAAAAUUUU | A4U4 | DraI |
| GGGUUU | G3U3 | DraI |

### Example 137-B

The synthesized multi-nucleotides described above in example 137-A were used for RNA stability testing described as follows.

### Accelerated RNA Stability Testing at 60° C.

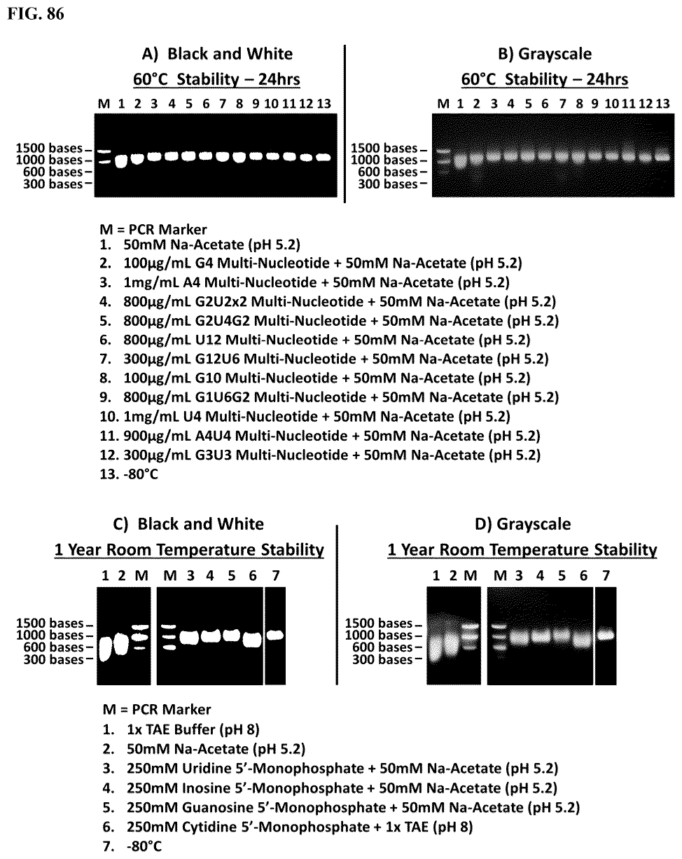

In vitro transcribed RNA was diluted at a ratio of about 1:10 (about 100-300 μg/mL) in different compositions containing 50 mM Na-Acetate (pH 5.2) or one or more compounds in compositions with 50 mM Na-Acetate (pH 5.2) as shown in FIGS. 86A and B.
M=PCR Marker
1. 50 mM Na-Acetate (pH 5.2)
2. 100 μg/mL G4 Multi-Nucleotide+50 mM Na-Acetate (pH 5.2)
3. 1 mg/mL A4 Multi-Nucleotide+50 mM Na-Acetate (pH 5.2)
4. 800 μg/mL G2U2×2 Multi-Nucleotide+50 mM Na-Acetate (pH 5.2)
5. 800 μg/mL G2U4G2 Multi-Nucleotide+50 mM Na-Acetate (pH 5.2)
6. 800 μg/mL U12 Multi-Nucleotide+50 mM Na-Acetate (pH 5.2)
7. 300 μg/mL G12U6 Multi-Nucleotide+50 mM Na-Acetate (pH 5.2)
8. 100 μg/mL G10 Multi-Nucleotide+50 mM Na-Acetate (pH 5.2)
9. 800 μg/mL G1U6G2 Multi-Nucleotide+50 mM Na-Acetate (pH 5.2)
10. 1 mg/mL U4 Multi-Nucleotide+50 mM Na-Acetate (pH 5.2)
11. 900 μg/mL A4U4 Multi-Nucleotide+50 mM Na-Acetate (pH 5.2)
12. 300 μg/mL G3U3 Multi-Nucleotide+50 mM Na-Acetate (pH 5.2)
13. −80° C.

Following dilution of each sample in each respective RNA storage environment, samples were stored in a thermal cycler at 60° C. for about 24 hours and then analyzed by denaturing agarose gel electrophoresis as shown in FIGS. 86A and B.

### Example 138

1 Year Room Temperature RNA Stability Testing (about 350-400 days)

In vitro transcribed RNA was diluted at a ratio of about 1:10 (about 100-300 μg/mL) in different compositions containing 1×TAE Buffer (pH 8) or 50 mM Na-Acetate (pH 5.2) or one or more compounds in compositions with 1×TAE Buffer (pH 8) or 50 mM Na-Acetate (pH 5.2) as shown in FIGS. 86 C and D.
M=PCR Marker
1. 1×TAE Buffer (pH 8)
2. 50 mM Na-Acetate (pH 5.2)
3. 250 mM Uridine 5′-Monophosphate+50 mM Na-Acetate (pH 5.2)
4. 250 mM Inosine 5′-Monophosphate+50 mM Na-Acetate (pH 5.2)
5. 250 mM Guanosine 5′-Monophosphate+50 mM Na-Acetate (pH 5.2)
6. 250 mM Cytidine 5′-Monophosphate+1×TAE (pH 8)
7. −80° C.

Following dilution of each sample in each respective RNA storage environment, samples were stored at room temperature (in a room with temperatures ranging from about 20° C. to 30° C.) over the course of about 1 year (about 350-400 days). Following storage for about 1 year, samples were then analyzed by denaturing agarose gel electrophoresis as shown in FIGS. 86 C and D.

Prior to analysis by agarose gel electrophoresis, samples were diluted 2× with water and adjusted to pH 7 with 1M Tris-HCl (pH 7) to help improve analysis by gel electrophoresis.

### Example 139

1 Year Room Temperature RNA Stability Testing (about 350-400 days)

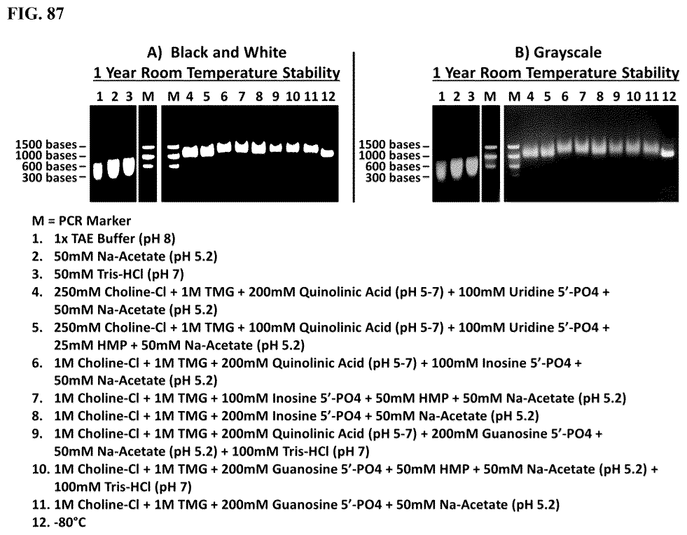

In vitro transcribed RNA was diluted at a ratio of about 1:10 (about 100-300 μg/mL) in different compositions containing 1×TAE Buffer (pH 8), 50 mM Na-Acetate (pH 5.2), or 50 mM Tris-HCl (pH 7) or various combinations of one or more RNA stabilizing substance with selected buffers as shown in FIGS. 87A and B.
M=PCR Marker
1. 1×TAE Buffer (pH 8)
2. 50 mM Na-Acetate (pH 5.2)
3. 50 mM Tris-HCl (pH 7)
4. 250 mM Choline-Cl+1M TMG+200 mM Quinolinic Acid (pH 5-7)+100 mM Uridine 5′-PO4+50 mM Na-Acetate (pH 5.2)
5. 250 mM Choline-Cl+1M TMG+100 mM Quinolinic Acid (pH 5-7)+100 mM Uridine 5′-PO4+25 mM HMP+50 mM Na-Acetate (pH 5.2)
6. 1M Choline-Cl+1M TMG+200 mM Quinolinic Acid (pH 5-7)+100 mM Inosine 5′-PO4 +50 mM Na-Acetate (pH 5.2)
7. 1M Choline-Cl+1M TMG+100 mM Inosine 5′-PO4+50 mM HMP+50 mM Na-Acetate (pH 5.2)
8. 1M Choline-Cl+1M TMG+200 mM Inosine 5′-PO4+50 mM Na-Acetate (pH 5.2)
9. 1M Choline-Cl+1M TMG+200 mM Quinolinic Acid (pH 5-7)+200 mM Guanosine 5′-PO4+50 mM Na-Acetate (pH 5.2)+100 mM Tris-HCl (pH 7)
10. 1M Choline-Cl+1M TMG+200 mM Guanosine 5′-PO4+50 mM HMP+50 mM Na-Acetate (pH 5.2)+100 mM Tris-HCl (pH 7)
11. 1M Choline-Cl+1M TMG+200 mM Guanosine 5′-PO4+50 mM Na-Acetate (pH 5.2)
12. −80° C.

Following dilution of each sample in each respective RNA storage environment, samples were stored at room temperature (in a room with temperatures ranging from about 20° C. to 30° C.) over the course of about 1 year (about 350-400 days). Following storage for about 1 year, samples were then analyzed by denaturing agarose gel electrophoresis as shown in FIGS. 87A and B.

Prior to analysis by agarose gel electrophoresis, samples were diluted 2× with water and adjusted to pH 7 with 1M Tris-HCl (pH 7) to help improve analysis by gel electrophoresis.

### Example 140

1 Year Room Temperature RNA Stability Testing (about 350-400 Days)

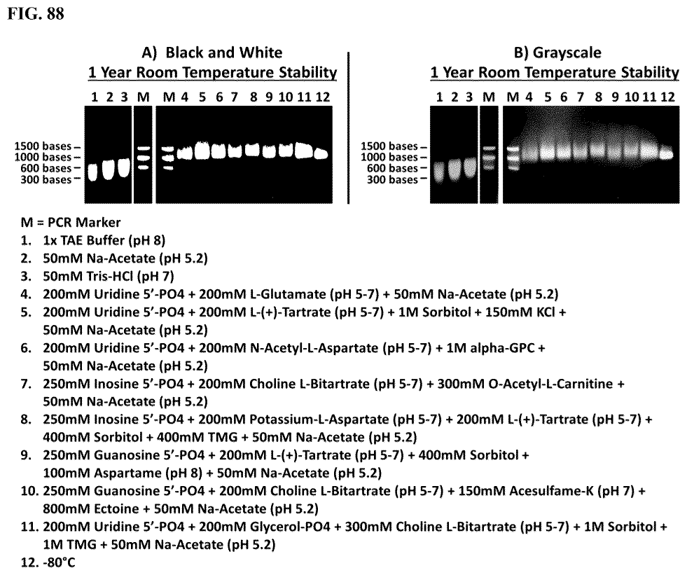

In vitro transcribed RNA was diluted at a ratio of about 1:10 (about 100-300 μg/mL) in different compositions containing 1×TAE Buffer (pH 8), 50 mM Na-Acetate (pH 5.2), or 50 mM Tris-HCl (pH 7) or various combinations of one or more RNA stabilizing substance with selected buffers as shown in FIGS. 88A and B.
M=PCR Marker
1. 1×TAE Buffer (pH 8)
2. 50 mM Na-Acetate (pH 5.2)
3. 50 mM Tris-HCl (pH 7)
4. 200 mM Uridine 5′-PO4+200 mM L-Glutamate (pH 5-7)+50 mM Na-Acetate (pH 5.2)
5. 200 mM Uridine 5′-PO4+200 mM L-(+)-Tartrate (pH 5-7)+1M Sorbitol+150 mM KCl+50 mM Na-Acetate (pH 5.2)
6. 200 mM Uridine 5′-PO4+200 mM N-Acetyl-L-Aspartate (pH 5-7)+1M alpha-GPC+50 mM Na-Acetate (pH 5.2)
7. 250 mM Inosine 5′-PO4+200 mM Choline L-Bitartrate (pH 5-7)+300 mM O-Acetyl-L-Carnitine (pH 5-7)+50 mM Na-Acetate (pH 5.2)
8. 250 mM Inosine 5′-PO4+200 mM Potassium-L-Aspartate (pH 5-7)+200 mM L-(+)-Tartrate (pH 5-7)+400 mM Sorbitol+400 mM TMG+50 mM Na-Acetate (pH 5.2)
9. 250 mM Guanosine 5′-PO4+200 mM L-(+)-Tartrate (pH 5-7)+400 mM Sorbitol+100 mM Aspartame (pH 8)+50 mM Na-Acetate (pH 5.2)
10. 250 mM Guanosine 5′-PO4+200 mM Choline L-Bitartrate (pH 5-7)+150 mM Acesulfame-K (pH 7)+800 mM Ectoine+50 mM Na-Acetate (pH 5.2)
11. 200 mM Uridine 5′-PO4+200 mM Glycerol-PO4+300 mM Choline L-Bitartrate (pH 5-7)+1M Sorbitol+1M TMG+50 mM Na-Acetate (pH 5.2)
12. −80° C.

Following dilution of each sample in each respective RNA storage environment, samples were stored at room temperature (in a room with temperatures ranging from about 20° C. to 30° C.) over the course of about 1 year (about 350-400 days). Following storage for about 1 year, samples were then analyzed by denaturing agarose gel electrophoresis as shown in FIGS. 88A and B.

Prior to analysis by agarose gel electrophoresis, samples were diluted 2× with water and adjusted to pH 7 with 1M Tris-HCl (pH 7) to help improve analysis by gel electrophoresis.

The pH of O-Acetyl-L-Carnitine was adjusted to about 5-7 using NaOH prior to being added to respective compositions.

The following substances in one or more compositions were purchased as follows: O-Acetyl-L-Carnitine (also known as Acetyl L-Carnitine) (NutriVita Shop (NVS), Lake Forest, CA)

### Example 141

1 Year Room Temperature RNA Stability Testing (about 350-400 days)

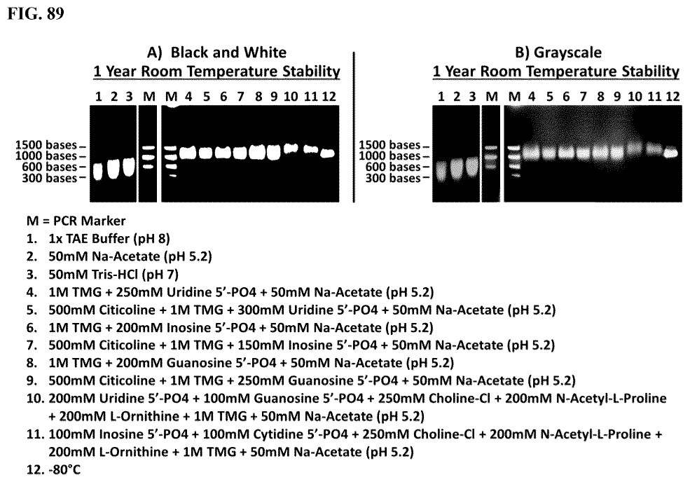

In vitro transcribed RNA was diluted at a ratio of about 1:10 (about 100-300 μg/mL) in different compositions containing 1×TAE Buffer (pH 8), 50 mM Na-Acetate (pH 5.2), or 50 mM Tris-HCl (pH 7) or various combinations of one or more RNA stabilizing substance with selected buffers as shown in FIGS. 89A and B.
M=PCR Marker
1. 1×TAE Buffer (pH 8)
2. 50 mM Na-Acetate (pH 5.2)
3. 50 mM Tris-HCl (pH 7)
4. 1M TMG+250 mM Uridine 5′-PO4+50 mM Na-Acetate (pH 5.2)
5. 500 mM Citicoline+1M TMG+300 mM Uridine 5′-PO4+50 mM Na-Acetate (pH 5.2)
6. 1M TMG+200 mM Inosine 5′-PO4+50 mM Na-Acetate (pH 5.2)
7. 500 mM Citicoline+1M TMG+150 mM Inosine 5′-PO4+50 mM Na-Acetate (pH 5.2)
8. 1M TMG+200 mM Guanosine 5′-PO4+50 mM Na-Acetate (pH 5.2)
9. 500 mM Citicoline+1M TMG+250 mM Guanosine 5′-PO4+50 mM Na-Acetate (pH 5.2)
10. 200 mM Uridine 5′-PO4+100 mM Guanosine 5′-PO4+250 mM Choline-Cl+200 mM N-Acetyl-L-Proline+200 mM L-Ornithine+1M TMG+50 mM Na-Acetate (pH 5.2)
11. 100 mM Inosine 5′-PO4+100 mM Cytidine 5′-PO4+250 mM Choline-Cl+200 mM N-Acetyl-L-Proline+200 mM L-Ornithine+1M TMG+50 mM Na-Acetate (pH 5.2)
12. −80° C.

Following dilution of each sample in each respective RNA storage environment, samples were stored at room temperature (in a room with temperatures ranging from about 20° C. to 30° C.) over the course of about 1 year (about 350-400 days). Following storage for about 1 year, samples were then analyzed by denaturing agarose gel electrophoresis as shown in FIGS. 89A and B.

Prior to analysis by agarose gel electrophoresis, samples were diluted 2× with water and adjusted to pH 7 with 1M Tris-HCl (pH 7) to help improve analysis by gel electrophoresis.

### Example 142

1 Year Room Temperature RNA Stability Testing (about 350-400 Days)

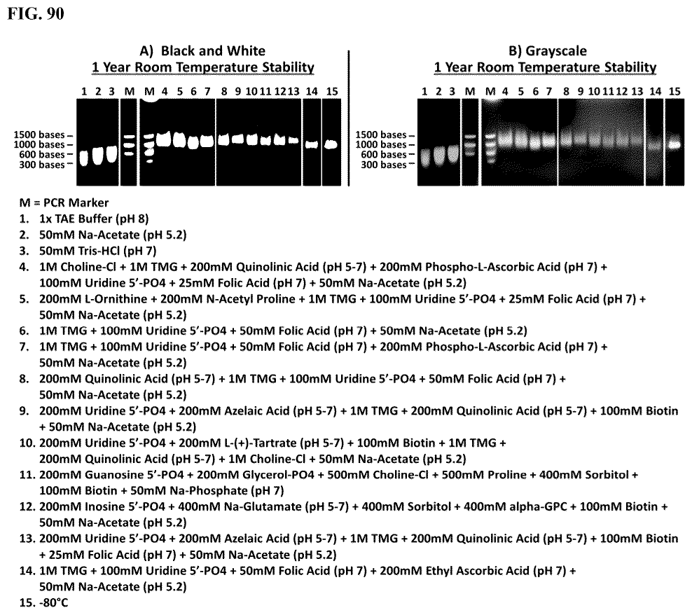

In vitro transcribed RNA was diluted at a ratio of about 1:10 (about 100-300 μg/mL) in different compositions containing 1×TAE Buffer (pH 8), 50 mM Na-Acetate (pH 5.2), or 50 mM Tris-HCl (pH 7) or various combinations of one or more RNA stabilizing substance with selected buffers as shown in FIGS. 90A and B.
M=PCR Marker
1. 1×TAE Buffer (pH 8)
2. 50 mM Na-Acetate (pH 5.2)
3. 50 mM Tris-HCl (pH 7)
4. 1M Choline-Cl+1M TMG+200 mM Quinolinic Acid (pH 5-7)+200 mM Phospho-L-Ascorbic Acid (pH 7)+100 mM Uridine 5′-PO4+25 mM Folic Acid (pH 7)+50 mM Na-Acetate (pH 5.2)
5. 200 mM L-Ornithine+200 mM N-Acetyl Proline+1M TMG+100 mM Uridine 5′-PO₄₊₂₅ mM Folic Acid (pH 7)+50 mM Na-Acetate (pH 5.2)
6. 1M TMG+100 mM Uridine 5′-PO4+50 mM Folic Acid (pH 7)+50 mM Na-Acetate (pH 5.2)
7. 1M TMG+100 mM Uridine 5′-PO4+50 mM Folic Acid (pH 7)+200 mM Phospho-L-Ascorbic Acid (pH 7)+50 mM Na-Acetate (pH 5.2)
8. 200 mM Quinolinic Acid (pH 5-7)+1M TMG+100 mM Uridine 5′-PO4+50 mM Folic Acid (pH 7)+50 mM Na-Acetate (pH 5.2)
9. 200 mM Uridine 5′-PO4+200 mM Azelaic Acid (pH 5-7)+1M TMG+200 mM Quinolinic Acid (pH 5-7)+100 mM Biotin+50 mM Na-Acetate (pH 5.2)
10. 200 mM Uridine 5′-PO4+200 mM L-(+)-Tartrate (pH 5-7)+100 mM Biotin+1M TMG+200 mM Quinolinic Acid (pH 5-7)+1M Choline-Cl+50 mM Na-Acetate (pH 5.2)
11. 200 mM Guanosine 5′-PO4+200 mM Glycerol-PO4+500 mM Choline-Cl+500 mM Proline+400 mM Sorbitol+100 mM Biotin+50 mM Na-Phosphate (pH 7)
12. 200 mM Inosine 5′-PO4+400 mM Na-Glutamate (pH 5-7)+400 mM Sorbitol+400 mM alpha-GPC+100 mM Biotin+50 mM Na-Acetate (pH 5.2)
13. 200 mM Uridine 5′-PO4+200 mM Azelaic Acid (pH 5-7)+1M TMG+200 mM Quinolinic Acid (pH 5-7)+100 mM Biotin+25 mM Folic Acid (pH 7)+50 mM Na-Acetate (pH 5.2)
14. 1M TMG+100 mM Uridine 5′-PO4+50 mM Folic Acid (pH 7)+200 mM Ethyl Ascorbic Acid (pH 7)+50 mM Na-Acetate (pH 5.2)
15. −80° C.

Following dilution of each sample in each respective RNA storage environment, samples were stored at room temperature (in a room with temperatures ranging from about 20° C. to 30° C.) over the course of about 1 year (about 350-400 days). Following storage for about 1 year, samples were then analyzed by denaturing agarose gel electrophoresis as shown in FIGS. 90A and B.

Prior to analysis by agarose gel electrophoresis, samples were diluted 2× with water, adjusted to pH 7 with 1M Tris-HCl (pH 7), and centrifuged to remove residual undissolved material to help improve analysis by gel electrophoresis. The agarose gel was also imaged at about 470-490 nm to help improve sample analysis.

### Example 143-A

Accelerated RNA Stability Testing with Lipid Nanoparticles at 60° C.

In vitro transcribed RNA was encapsulated in lipid nanoparticles by first preparing a 5 mM Na-Acetate (pH 5.2) RNA aqueous phase with an RNA concentration of about 0.5-1 mg/mL. Next, a lipid ethanol phase was prepared by mixing 25 mM ionizable lipid SM-102, 5 mM neutral lipid DSPC, 19.25 mM cholesterol, and 0.75 mM PEG-lipid DMG-PEG2000 in ethanol to produce a molar ratio of 50:10:38.5:1.5, respectively (ionizable-lipid:neutral-lipid:cholesterol:PEG-lipid). The prepared RNA aqueous phase (pH 5.2) and ethanol lipid phase were then rapidly mixed at a ratio of about 3:1 aqueous RNA phase to ethanol lipid phase (e.g. 300 μL aqueous RNA phase mixed with 100 μL ethanol lipid phase) resulting in an N/P ratio of about 4:1. The two phases were mixed at flow rate of about 2 mL/min using a syringe pump and “Y” shaped microfluidic mixer with an inner diameter of about 0.05 inches. Following mixing of the aqueous and ethanol phases, this RNA/LNP mixture was then used to prepare selected compositions for RNA stability testing.

The following substances used to form lipid nanoparticles as described above were purchased as follows: SM-102 (Cayman Chemical, Product #33474), 1,2-distearoyl-sn-glycero-3-phosphatidylcholine (DSPC) (Cayman Chemical, Product #15100), Cholesterol (Cayman Chemical, Product #9003100), 1,2-Dimyristoyl-rac-glycero-3-methoxypolyethylene glycol-2000 (DMG-PEG (2000)) (Cayman Chemical, Product #33945).

### Example 143-B

Accelerated RNA Stability Testing with Lipid Nanoparticles at 60° C.

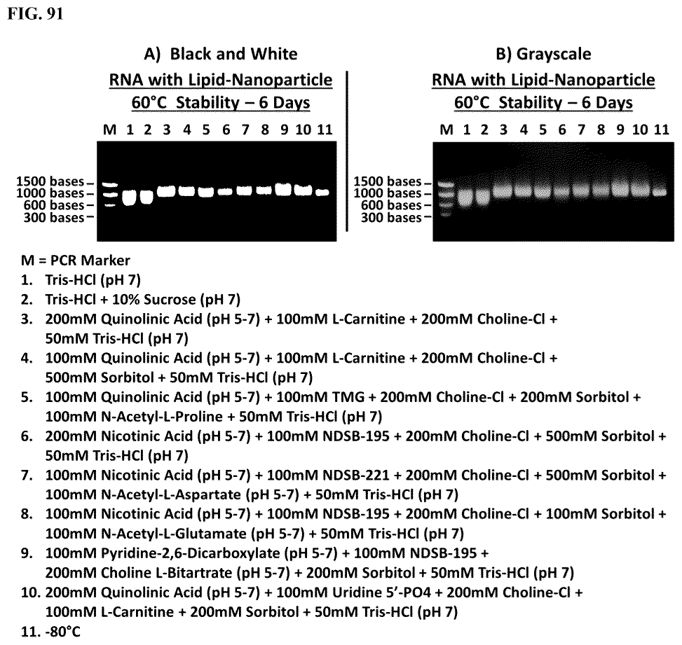

RNA/LNPs prepared as described above in Example 143-A were diluted at a ratio of about 1:5 (about 100-200 μg/mL RNA) in different compositions containing 50 mM Tris-HCl (pH 7) or 10% sucrose+50 mM Tris-HCl (pH 7) or one or more compounds in compositions with 50 mM Tris-HCl (pH 7) as shown in FIGS. 91A and B.
M=PCR Marker
1. Tris-HCl (pH 7)
2. Tris-HCl+10% Sucrose (pH 7)
3. 200 mM Quinolinic Acid (pH 5-7)+100 mM L-Carnitine+200 mM Choline-Cl+50 mM Tris-HCl (pH 7)
4. 100 mM Quinolinic Acid (pH 5-7)+100 mM L-Carnitine+200 mM Choline-Cl+500 mM Sorbitol+50 mM Tris-HCl (pH 7)
5. 100 mM Quinolinic Acid (pH 5-7)+100 mM TMG+200 mM Choline-Cl+200 mM Sorbitol+100 mM N-Acetyl-L-Proline+50 mM Tris-HCl (pH 7)
6. 200 mM Nicotinic Acid (pH 5-7)+100 mM NDSB-195+200 mM Choline-Cl+500 mM Sorbitol+50 mM Tris-HCl (pH 7)
7. 100 mM Nicotinic Acid (pH 5-7)+100 mM NDSB-221+200 mM Choline-Cl+500 mM Sorbitol+100 mM N-Acetyl-L-Aspartate (pH 5-7)+50 mM Tris-HCl (pH 7)
8. 100 mM Nicotinic Acid (pH 5-7)+100 mM NDSB-195+200 mM Choline-Cl+100 mM Sorbitol+100 mM N-Acetyl-L-Glutamate (pH 5-7)+50 mM Tris-HCl (pH 7)
9. 100 mM Pyridine-2,6-Dicarboxylate (pH 5-7)+100 mM NDSB-195+200 mM Choline L-Bitartrate (pH 5-7)+200 mM Sorbitol+50 mM Tris-HCl (pH 7)
10. 200 mM Quinolinic Acid (pH 5-7)+100 mM Uridine 5′-PO4+200 mM Choline-Cl+100 mM L-Carnitine+200 mM Sorbitol+50 mM Tris-HCl (pH 7)
11. −80° C.

Following dilution of each sample in each respective RNA/LNP storage environment, samples were stored in a thermal cycler at 60° C. for about 6 days and then analyzed by denaturing agarose gel electrophoresis as shown in FIGS. 91A and B.

Prior to analysis by agarose gel electrophoresis, samples were mixed at a 1:1 ratio with a 50% DMSO, 47.5% Formamide mixture supplemented with 0.4% SDS (resulting in a final sample concentration of about 25% DMSO, 23.75% Formamide, 0.2% SDS) and heated to 70° C. for about 2-4 min to dissociate the mRNA from LNPs. Following heating, samples were then centrifuged to remove residual undissolved material and analyzed by denaturing agarose gel electrophoresis.

### Example 144

Accelerated RNA Stability Testing with Lipid Nanoparticles at 60° C.

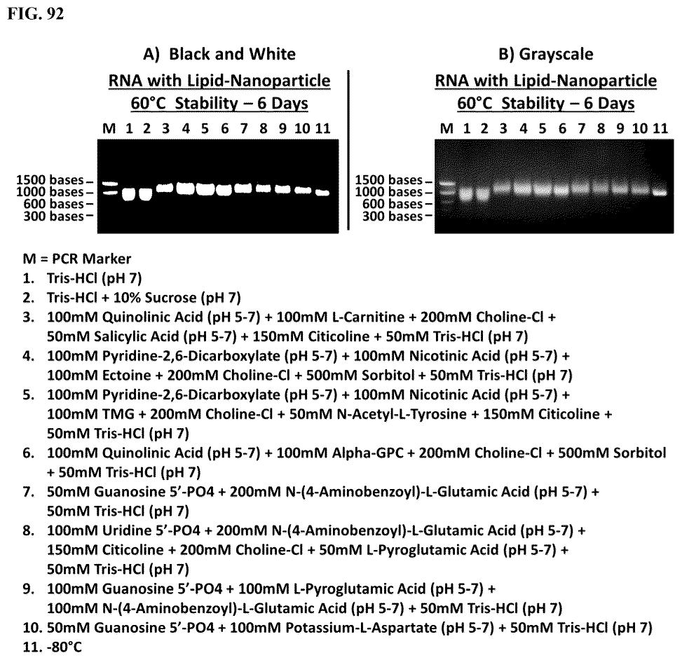

RNA/LNPs prepared as described above in Example 143-A were diluted at a ratio of about 1:5 (about 100-200 μg/mL RNA) in different compositions containing 50 mM Tris-HCl (pH 7) or 10% sucrose+50 mM Tris-HCl (pH 7) or one or more compounds in compositions with 50 mM Tris-HCl (pH 7) as shown in FIGS. 92A and B.
M=PCR Marker
1. Tris-HCl (pH 7)
2. Tris-HCl+10% Sucrose (pH 7)
3. 100 mM Quinolinic Acid (pH 5-7)+100 mM L-Carnitine+200 mM Choline-Cl+50 mM Salicylic Acid (pH 5-7)+150 mM Citicoline+50 mM Tris-HCl (pH 7)
4. 100 mM Pyridine-2,6-Dicarboxylate (pH 5-7)+100 mM Nicotinic Acid (pH 5-7)+100 mM Ectoine+200 mM Choline-Cl+500 mM Sorbitol+50 mM Tris-HCl (pH 7)
5. 100 mM Pyridine-2,6-Dicarboxylate (pH 5-7)+100 mM Nicotinic Acid (pH 5-7)+100 mM TMG+200 mM Choline-Cl+50 mM N-Acetyl-L-Tyrosine+150 mM Citicoline+50 mM Tris-HCl (pH 7)
6. 100 mM Quinolinic Acid (pH 5-7)+100 mM Alpha-GPC+200 mM Choline-Cl+500 mM Sorbitol+50 mM Tris-HCl (pH 7)
7. 50 mM Guanosine 5′-PO4+200 mM N-(4-Aminobenzoyl)-L-Glutamic Acid (pH 5-7)+50 mM Tris-HCl (pH 7)
8. 100 mM Uridine 5′-PO4+200 mM N-(4-Aminobenzoyl)-L-Glutamic Acid (pH 5-7)+150 mM Citicoline+200 mM Choline-Cl+50 mM L-Pyroglutamic Acid (pH 5-7)+50 mM Tris-HCl (pH 7)
9. 100 mM Guanosine 5′-PO4+100 mM L-Pyroglutamic Acid (pH 5-7)+100 mM N-(4-Aminobenzoyl)-L-Glutamic Acid (pH 5-7)+50 mM Tris-HCl (pH 7)
10. 50 mM Guanosine 5′-PO4+100 mM Potassium-L-Aspartate (pH 5-7)+50 mM Tris-HCl (pH 7)
11. −80° C.

Following dilution of each sample in each respective RNA/LNP storage environment, samples were stored in a thermal cycler at 60° C. for about 6 days and then analyzed by denaturing agarose gel electrophoresis as shown in FIGS. 92A and B.

Prior to analysis by agarose gel electrophoresis, samples were mixed at a 1:1 ratio with a 50% DMSO, 47.5% Formamide mixture supplemented with 0.4% SDS (resulting in a final sample concentration of about 25% DMSO, 23.75% Formamide, 0.2% SDS) and heated to 70° C. for about 2-4 min to dissociate the mRNA from LNPs. Following heating, samples were then centrifuged to remove residual undissolved material and analyzed by denaturing agarose gel electrophoresis. The agarose gel was also imaged at about 470-490 nm to help improve sample analysis.

### Example 145

Room Temperature RNA Stability Testing with Lipid Nanoparticles

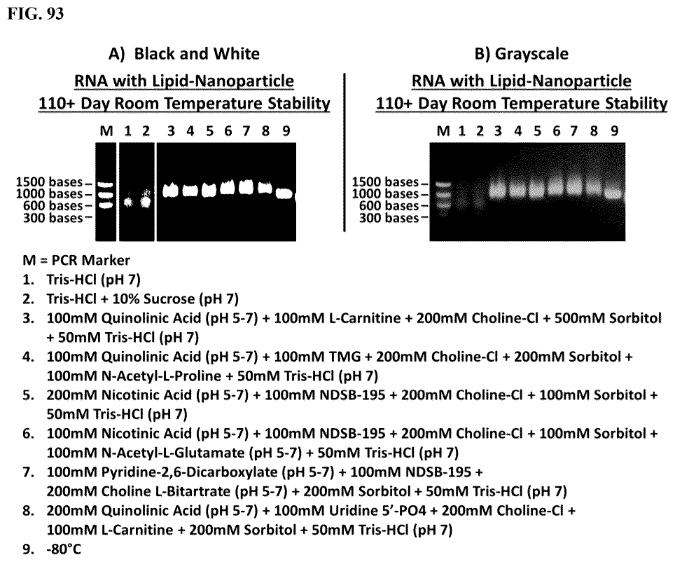

RNA/LNPs prepared as described above in Example 143-A were diluted at a ratio of about 1:5 (about 100-200 μg/mL RNA) in different compositions containing 50 mM Tris-HCl (pH 7) or 10% sucrose+50 mM Tris-HCl (pH 7) or one or more compounds in compositions with 50 mM Tris-HCl (pH 7) as shown in FIGS. 93A and B.
M=PCR Marker
1. Tris-HCl (pH 7)
2. Tris-HCl+10% Sucrose (pH 7)
3. 100 mM Quinolinic Acid (pH 5-7)+100 mM L-Carnitine+200 mM Choline-Cl+500 mM Sorbitol+50 mM Tris-HCl (pH 7)
4. 100 mM Quinolinic Acid (pH 5-7)+100 mM TMG+200 mM Choline-Cl+200 mM Sorbitol+100 mM N-Acetyl-L-Proline+50 mM Tris-HCl (pH 7)
5. 200 mM Nicotinic Acid (pH 5-7)+100 mM NDSB-195+200 mM Choline-Cl+100 mM Sorbitol+50 mM Tris-HCl (pH 7)
6. 100 mM Nicotinic Acid (pH 5-7)+100 mM NDSB-195+200 mM Choline-Cl+100 mM Sorbitol+100 mM N-Acetyl-L-Glutamate (pH 5-7)+50 mM Tris-HCl (pH 7)
7. 100 mM Pyridine-2,6-Dicarboxylate (pH 5-7)+100 mM NDSB-195+200 mM Choline L-Bitartrate (pH 5-7)+200 mM Sorbitol+50 mM Tris-HCl (pH 7)
8. 200 mM Quinolinic Acid (pH 5-7)+100 mM Uridine 5′-PO4+200 mM Choline-Cl+100 mM L-Carnitine+200 mM Sorbitol+50 mM Tris-HCl (pH 7)
9. −80° C.

Following dilution of each sample in each respective RNA/LNP storage environment, samples were stored at room temperature (in a room with temperatures ranging from about 20° C. to 30° C.) over the course of about 110-120 days. Following storage for about 110-120 days, samples were then analyzed by denaturing agarose gel electrophoresis as shown in FIGS. 93A and B.

Prior to analysis by agarose gel electrophoresis, samples were mixed at a 1:1 ratio with a 50% DMSO, 47.5% Formamide mixture supplemented with 0.4% SDS (resulting in a final sample concentration of about 25% DMSO, 23.75% Formamide, 0.2% SDS) and heated to 70° C. for about 2-4 min to dissociate the mRNA from LNPs. Following heating, samples were then centrifuged to remove residual undissolved material and analyzed by denaturing agarose gel electrophoresis.

### Example 146-A

### Synthesis of Codon Optimized GFP RNA for RNA Stability Testing

An additional RNA construct, herein referred to as “Codon Optimized GFP”, was synthesized as described in Example 6 above. Briefly, Codon Optimized GFP RNA was synthesized by in vitro transcription from a linear DNA construct with an upstream T7 RNA Polymerase promoter followed by the coding sequence for gene of interest. In vitro transcription was performed using the HiScribe T7 Quick High Yield RNA Synthesis Kit (New England Biolabs, Ipswich, MA; Product #E2050) according to the manufacturer's directions. Following in vitro transcription, the RNA was purified using a Monarch RNA Cleanup Kit (New England Biolabs, Ipswich, MA; Product #T2050) according to the manufacturer's directions. The purified RNA was then stored in molecular biology grade water at −80° C. and used for future RNA stability testing.

RNA concentration was measured by absorbance of the purified RNA at 260 nm using a Nanodrop ND-1000 (Thermo Fisher Scientific, Waltham, MA). Typical RNA concentration following in vitro transcription and purification was about 2-5 mg/mL.

### Example 146-B

### Codon Optimized GFP RNA Accelerated Stability Testing at 60° C.

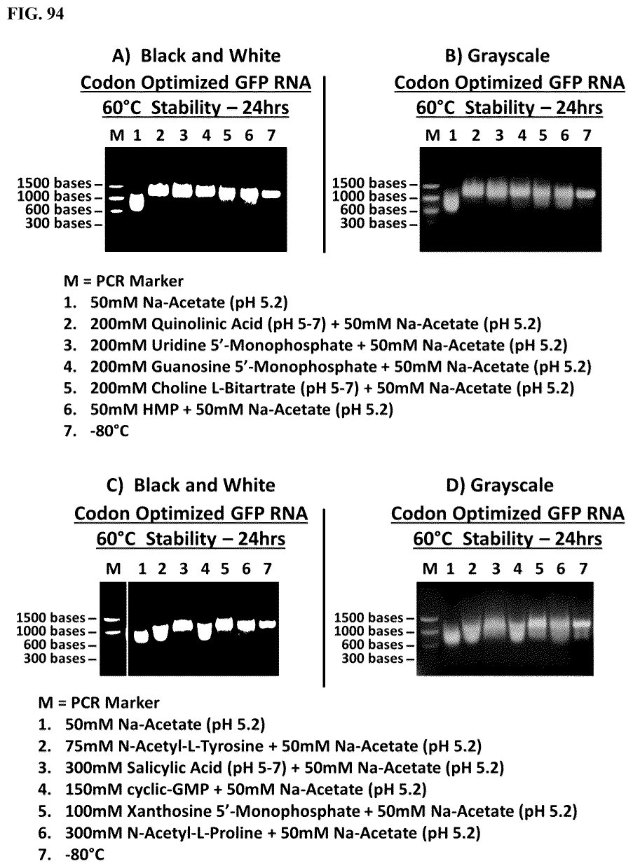

In vitro transcribed Codon Optimized GFP RNA as described in Example 146-A above was diluted at a ratio of about 1:5 (about 400-1000 μg/mL) in different compositions containing 50 mM Na-Acetate (pH 5.2) or one or more compounds in compositions with 50 mM Na-Acetate (pH 5.2) as shown in FIGS. 94A and B.
M=PCR Marker
1. 50 mM Na-Acetate (pH 5.2)
2. 200 mM Quinolinic Acid (pH 5-7)+50 mM Na-Acetate (pH 5.2)
3. 200 mM Uridine 5′-Monophosphate+50 mM Na-Acetate (pH 5.2)
4. 200 mM Guanosine 5′-Monophosphate+50 mM Na-Acetate (pH 5.2)
5. 200 mM Choline L-Bitartrate (pH 5-7)+50 mM Na-Acetate (pH 5.2)
6. 50 mM HMP+50 mM Na-Acetate (pH 5.2)
7. −80° C.

Following dilution of each sample in each respective RNA storage environment, samples were stored in a thermal cycler at 60° C. for about 24 hours and then analyzed by denaturing agarose gel electrophoresis as shown in FIGS. 94A and B.

### Example 147

### Codon Optimized GFP RNA Accelerated Stability Testing at 60° C.

In vitro transcribed Codon Optimized GFP RNA as described in Example 146-A above was diluted at a ratio of about 1:5 (about 400-1000 μg/mL) in different compositions containing 50 mM Na-Acetate (pH 5.2) or one or more compounds in compositions with 50 mM Na-Acetate (pH 5.2) as shown in FIGS. 94 C and D.
M=PCR Marker
1. 50 mM Na-Acetate (pH 5.2)
2. 75 mM N-Acetyl-L-Tyrosine+50 mM Na-Acetate (pH 5.2)
3. 300 mM Salicylic Acid (pH 5-7)+50 mM Na-Acetate (pH 5.2)
4. 150 mM cyclic-GMP+50 mM Na-Acetate (pH 5.2)
5. 100 mM Xanthosine 5′-Monophosphate+50 mM Na-Acetate (pH 5.2)
6. 300 mM N-Acetyl-L-Proline+50 mM Na-Acetate (pH 5.2)
7. −80° C.

Following dilution of each sample in each respective RNA storage environment, samples were stored in a thermal cycler at 60° C. for about 24 hours and then analyzed by denaturing agarose gel electrophoresis as shown in FIGS. 94 C and D.

Prior to analysis by agarose gel electrophoresis, samples were diluted 2× with water to help improve analysis by gel electrophoresis. The agarose gel was also imaged at about 470-490 nm to help improve sample analysis.

### Example 148

### Codon Optimized GFP RNA Accelerated Stability Testing at 60° C.

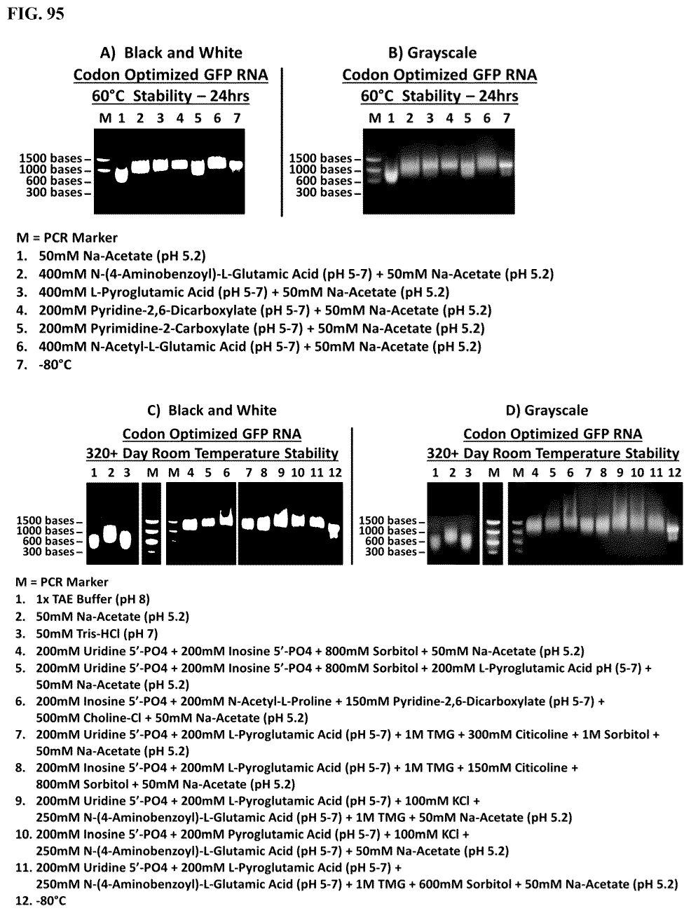

In vitro transcribed Codon Optimized GFP RNA as described in Example 146-A above was diluted at a ratio of about 1:5 (about 400-1000 μg/mL) in different compositions containing 50 mM Na-Acetate (pH 5.2) or one or more compounds in compositions with 50 mM Na-Acetate (pH 5.2) as shown in FIGS. 95A and B.
M=PCR Marker
1. 50 mM Na-Acetate (pH 5.2)
2. 400 mM N-(4-Aminobenzoyl)-L-Glutamic Acid (pH 5-7)+50 mM Na-Acetate (pH 5.2)
3. 400 mM L-Pyroglutamic Acid (pH 5-7)+50 mM Na-Acetate (pH 5.2)
4. 200 mM Pyridine-2,6-Dicarboxylate (pH 5-7)+50 mM Na-Acetate (pH 5.2)
5. 200 mM Pyrimidine-2-Carboxylate (pH 5-7)+50 mM Na-Acetate (pH 5.2)
6. 400 mM N-Acetyl-L-Glutamic Acid (pH 5-7)+50 mM Na-Acetate (pH 5.2)
7. −80° C.

Following dilution of each sample in each respective RNA storage environment, samples were stored in a thermal cycler at 60° C. for about 24 hours and then analyzed by denaturing agarose gel electrophoresis as shown in FIGS. 95A and B.

### Example 149

Room Temperature Codon Optimized GFP RNA Stability Testing (about 320-360 Days)

In vitro transcribed Codon Optimized GFP RNA as described in Example 146-A above was diluted at a ratio of about 1:5 (about 400-1000 μg/mL) in different compositions containing 1×TAE Buffer (pH 8), 50 mM Na-Acetate (pH 5.2), or 50 mM Tris-HCl (pH 7) or various combinations of one or more RNA stabilizing substance with selected buffers as shown in FIGS. 95 C and D.
M=PCR Marker
1. 1×TAE Buffer (pH 8)
2. 50 mM Na-Acetate (pH 5.2)
3. 50 mM Tris-HCl (pH 7)
4. 200 mM Uridine 5′-PO4+200 mM Inosine 5′-PO4+800 mM Sorbitol+50 mM Na-Acetate (pH 5.2)
5. 200 mM Uridine 5′-PO4+200 mM Inosine 5′-PO4+800 mM Sorbitol+200 mM L-Pyroglutamic Acid pH (5-7)+50 mM Na-Acetate (pH 5.2)
6. 200 mM Inosine 5′-PO4+200 mM N-Acetyl-L-Proline+150 mM Pyridine-2,6-Dicarboxylate (pH 5-7)+500 mM Choline-Cl+50 mM Na-Acetate (pH 5.2)
7. 200 mM Uridine 5′-PO4+200 mM L-Pyroglutamic Acid (pH 5-7)+1M TMG+300 mM Citicoline+1M Sorbitol+50 mM Na-Acetate (pH 5.2)
8. 200 mM Inosine 5′-PO4+200 mM L-Pyroglutamic Acid (pH 5-7)+1M TMG+150 mM Citicoline+800 mM Sorbitol+50 mM Na-Acetate (pH 5.2)
9. 200 mM Uridine 5′-PO4+200 mM L-Pyroglutamic Acid (pH5-7)+100 mM KCl+250 mM N-(4-Aminobenzoyl)-L-Glutamic Acid (pH 5-7)+1M TMG+50 mM Na-Acetate (pH 5.2)
10. 200 mM Inosine 5′-PO4+200 mM Pyroglutamic Acid (pH 5-7)+100 mM KCl+250 mM N-(4-Aminobenzoyl)-L-Glutamic Acid (pH 5-7)+50 mM Na-Acetate (pH 5.2)
11. 200 mM Uridine 5′-PO4+200 mM L-Pyroglutamic Acid (pH 5-7)+250 mM N-(4-Aminobenzoyl)-L-Glutamic Acid (pH 5-7)+1M TMG+600 mM Sorbitol+50 mM Na-Acetate (pH 5.2)
12. −80° C.

Following dilution of each sample in each respective RNA storage environment, samples were stored at room temperature (in a room with temperatures ranging from about 20° C. to 30° C.) over the course of about 320-360 days. Following storage for about 320-360 days, samples were then analyzed by denaturing agarose gel electrophoresis as shown in FIGS. 95 C and D.

Prior to analysis by agarose gel electrophoresis, samples were diluted 2× with water and adjusted to pH 7 with 1M Tris-HCl (pH 7) to help improve analysis by gel electrophoresis.

### Example 150

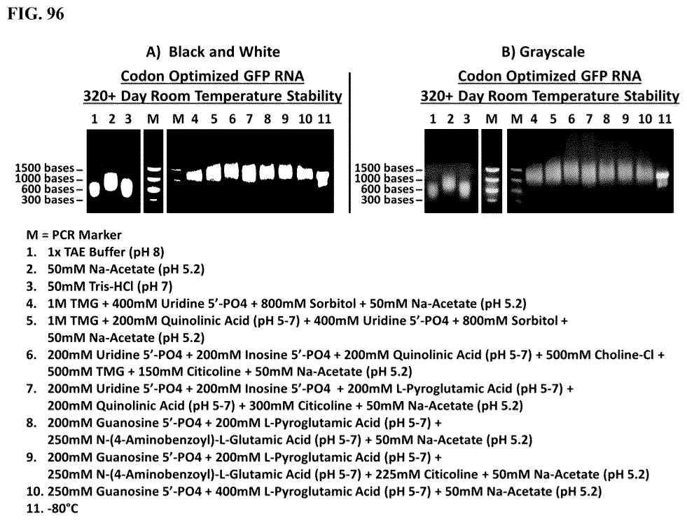

Room Temperature Codon Optimized GFP RNA Stability Testing (about 320-360 days) In vitro transcribed Codon Optimized GFP RNA as described in Example 146-A above was diluted at a ratio of about 1:5 (about 400-1000 μg/mL) in different compositions containing 1×TAE Buffer (pH 8), 50 mM Na-Acetate (pH 5.2), or 50 mM Tris-HCl (pH 7) or various combinations of one or more RNA stabilizing substance with selected buffers as shown in FIGS. 96 A and B.
M=PCR Marker
1. 1×TAE Buffer (pH 8)
2. 50 mM Na-Acetate (pH 5.2)
3. 50 mM Tris-HCl (pH 7)
4. 1M TMG+400 mM Uridine 5′-PO4+800 mM Sorbitol+50 mM Na-Acetate (pH 5.2)
5. 1M TMG+200 mM Quinolinic Acid (pH 5-7)+400 mM Uridine 5′-PO4+800 mM Sorbitol+50 mM Na-Acetate (pH 5.2)
6. 200 mM Uridine 5′-PO4+200 mM Inosine 5′-PO4+200 mM Quinolinic Acid (pH 5-7)+500 mM Choline-Cl+500 mM TMG+150 mM Citicoline+50 mM Na-Acetate (pH 5.2)
7. 200 mM Uridine 5′-PO4+200 mM Inosine 5′-PO4+200 mM L-Pyroglutamic Acid (pH 5-7)+200 mM Quinolinic Acid (pH 5-7)+300 mM Citicoline+50 mM Na-Acetate (pH 5.2)
8. 200 mM Guanosine 5′-PO4+200 mM L-Pyroglutamic Acid (pH 5-7)+250 mM N-(4-Aminobenzoyl)-L-Glutamic Acid (pH 5-7)+50 mM Na-Acetate (pH 5.2)
9. 200 mM Guanosine 5′-PO4+200 mM L-Pyroglutamic Acid (pH 5-7)+250 mM N-(4-Aminobenzoyl)-L-Glutamic Acid (pH 5-7)+225 mM Citicoline+50 mM Na-Acetate (pH 5.2)
10. 250 mM Guanosine 5′-PO4+400 mM L-Pyroglutamic Acid (pH 5-7)+50 mM Na-Acetate (pH 5.2)
11. −80° C.

Following dilution of each sample in each respective RNA storage environment, samples were stored at room temperature (in a room with temperatures ranging from about 20° C. to 30° C.) over the course of about 320-360 days. Following storage for about 320-360 days, samples were then analyzed by denaturing agarose gel electrophoresis as shown in FIGS. 96A and B.

Prior to analysis by agarose gel electrophoresis, samples were diluted 2× with water and adjusted to pH 7 with 1M Tris-HCl (pH 7) to help improve analysis by gel electrophoresis. The agarose gel was also imaged at about 470-490 nm to help improve sample analysis.

### Example 151-A

### Synthesis of Firefly Luciferase RNA for RNA Stability Testing

An additional RNA construct, herein referred to as “Firefly Luciferase”, was synthesized as described in Example 6 above. Briefly, Firefly Luciferase RNA was synthesized by in vitro transcription from a linear DNA construct (provided as part of the New England Biolabs HiScribe T7 Quick High Yield RNA Synthesis Kit, Product #E2050, Fluc control template DNA, kit component #N0426AVIAL) with an upstream T7 RNA Polymerase promoter followed by the coding sequence for the gene of interest. In vitro transcription was performed using the HiScribe T7 Quick High Yield RNA Synthesis Kit (New England Biolabs, Ipswich, MA; Product #E2050) according to the manufacturer's directions. Following in vitro transcription, the RNA was purified using a Monarch RNA Cleanup Kit (New England Biolabs, Ipswich, MA; Product #T2050) according to the manufacturer's directions. The purified RNA was then stored in molecular biology grade water at −80° C. and used for future RNA stability testing.

RNA concentration was measured by absorbance of the purified RNA at 260 nm using a Nanodrop ND-1000 (Thermo Fisher Scientific, Waltham, MA). Typical RNA concentration following in vitro transcription and purification was about 2-5 mg/mL.

### Example 151-B

### Firefly Luciferase RNA Accelerated Stability Testing at 50° C.

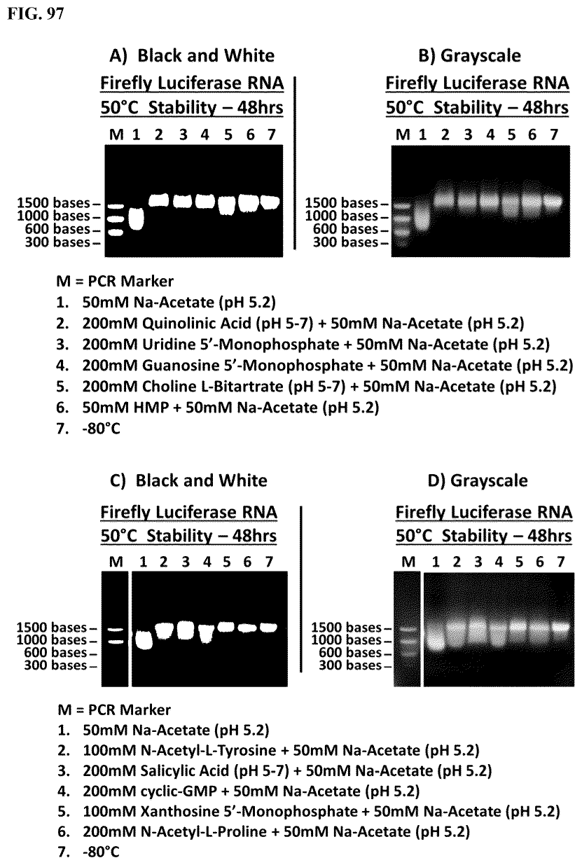

In vitro transcribed Firefly Luciferase RNA as described in Example 151-A above was diluted at a ratio of about 1:5 (about 400-1000 μg/mL) in different compositions containing 50 mM Na-Acetate (pH 5.2) or one or more compounds in compositions with 50 mM Na-Acetate (pH 5.2) as shown in FIGS. 97A and B.
M=PCR Marker
1. 50 mM Na-Acetate (pH 5.2)
2. 200 mM Quinolinic Acid (pH 5-7)+50 mM Na-Acetate (pH 5.2)
3. 200 mM Uridine 5′-Monophosphate+50 mM Na-Acetate (pH 5.2)
4. 200 mM Guanosine 5′-Monophosphate+50 mM Na-Acetate (pH 5.2)
5. 200 mM Choline L-Bitartrate (pH 5-7)+50 mM Na-Acetate (pH 5.2)
6. 50 mM HMP+50 mM Na-Acetate (pH 5.2)
7. −80° C.

Following dilution of each sample in each respective RNA storage environment, samples were stored in a thermal cycler at 50° C. for about 48 hours and then analyzed by denaturing agarose gel electrophoresis as shown in FIGS. 97A and B.

### Example 152

### Firefly Luciferase RNA Accelerated Stability Testing at 50° C.

In vitro transcribed Firefly Luciferase RNA as described in Example 151-A above was diluted at a ratio of about 1:5 (about 400-1000 μg/mL) in different compositions containing 50 mM Na-Acetate (pH 5.2) or one or more compounds in compositions with 50 mM Na-Acetate (pH 5.2) as shown in FIGS. 97 C and D.
M=PCR Marker
1. 50 mM Na-Acetate (pH 5.2)
2. 100 mM N-Acetyl-L-Tyrosine+50 mM Na-Acetate (pH 5.2)
3. 200 mM Salicylic Acid (pH 5-7)+50 mM Na-Acetate (pH 5.2)
4. 200 mM cyclic-GMP+50 mM Na-Acetate (pH 5.2)
5. 100 mM Xanthosine 5′-Monophosphate+50 mM Na-Acetate (pH 5.2)
6. 200 mM N-Acetyl-L-Proline+50 mM Na-Acetate (pH 5.2)
7. −80° C.

Following dilution of each sample in each respective RNA storage environment, samples were stored in a thermal cycler at 50° C. for about 48 hours and then analyzed by denaturing agarose gel electrophoresis as shown in FIGS. 97 C and D.

Prior to analysis by agarose gel electrophoresis, samples were diluted 2× with water to help improve analysis by gel electrophoresis. The agarose gel was also imaged at about 470-490 nm to help improve sample analysis.

### Example 153

### Firefly Luciferase RNA Accelerated Stability Testing at 50° C.

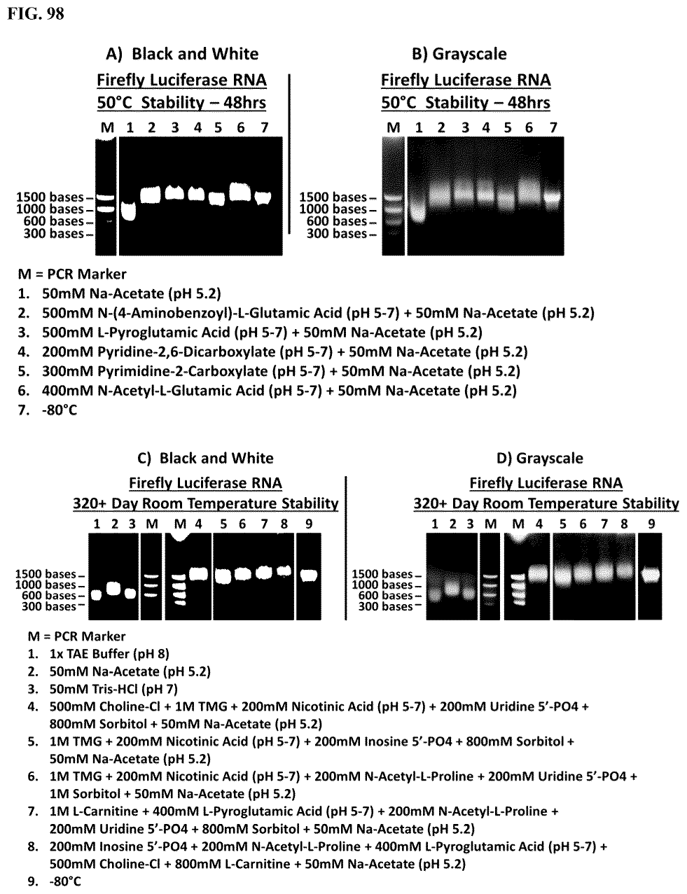

In vitro transcribed Firefly Luciferase RNA as described in Example 151-A above was diluted at a ratio of about 1:5 (about 400-1000 μg/mL) in different compositions containing 50 mM Na-Acetate (pH 5.2) or one or more compounds in compositions with 50 mM Na-Acetate (pH 5.2) as shown in FIGS. 98A and B.
M=PCR Marker
1. 50 mM Na-Acetate (pH 5.2)
2. 500 mM N-(4-Aminobenzoyl)-L-Glutamic Acid (pH 5-7)+50 mM Na-Acetate (pH 5.2)
3. 500 mM L-Pyroglutamic Acid (pH 5-7)+50 mM Na-Acetate (pH 5.2)
4. 200 mM Pyridine-2,6-Dicarboxylate (pH 5-7)+50 mM Na-Acetate (pH 5.2)
5. 300 mM Pyrimidine-2-Carboxylate (pH 5-7)+50 mM Na-Acetate (pH 5.2)
6. 400 mM N-Acetyl-L-Glutamic Acid (pH 5-7)+50 mM Na-Acetate (pH 5.2)
7. −80° C.

Following dilution of each sample in each respective RNA storage environment, samples were stored in a thermal cycler at 50° C. for about 48 hours and then analyzed by denaturing agarose gel electrophoresis as shown in FIGS. 98A and B.

### Example 154

Room Temperature Firefly Luciferase RNA Stability Testing (about 320-360 days)

In vitro transcribed Firefly Luciferase RNA as described in Example 151-A above was diluted at a ratio of about 1:5 (about 400-1000 μg/mL) in different compositions containing 1×TAE Buffer (pH 8), 50 mM Na-Acetate (pH 5.2), or 50 mM Tris-HCl (pH 7) or various combinations of one or more RNA stabilizing substance with selected buffers as shown in FIGS. 98 C and D.
M=PCR Marker
1. 1×TAE Buffer (pH 8)
2. 50 mM Na-Acetate (pH 5.2)
3. 50 mM Tris-HCl (pH 7)
4. 500 mM Choline-Cl+1M TMG+200 mM Nicotinic Acid (pH 5-7)+200 mM Uridine 5′-PO4+800 mM Sorbitol+50 mM Na-Acetate (pH 5.2)
5. 1M TMG+200 mM Nicotinic Acid (pH 5-7)+200 mM Inosine 5′-PO4+800 mM Sorbitol+50 mM Na-Acetate (pH 5.2)
6. 1M TMG+200 mM Nicotinic Acid (pH 5-7)+200 mM N-Acetyl-L-Proline+200 mM Uridine 5′-PO4+1M Sorbitol+50 mM Na-Acetate (pH 5.2)
7. 1M L-Carnitine+400 mM L-Pyroglutamic Acid (pH 5-7)+200 mM N-Acetyl-L-Proline+200 mM Uridine 5′-PO4+800 mM Sorbitol+50 mM Na-Acetate (pH 5.2)
8. 200 mM Inosine 5′-PO4+200 mM N-Acetyl-L-Proline+400 mM L-Pyroglutamic Acid (pH 5-7)+500 mM Choline-Cl+800 mM L-Carnitine+50 mM Na-Acetate (pH 5.2)
9. −80° C.

Following dilution of each sample in each respective RNA storage environment, samples were stored at room temperature (in a room with temperatures ranging from about 20° C. to 30° C.) over the course of about 320-360 days. Following storage for about 320-360 days, samples were then analyzed by denaturing agarose gel electrophoresis as shown in FIGS. 98 C and D.

Prior to analysis by agarose gel electrophoresis, samples were diluted 2× with water and adjusted to pH 7 with 1M Tris-HCl (pH 7) to help improve analysis by gel electrophoresis.

### Example 155

Room Temperature Firefly Luciferase RNA Stability Testing (about 320-360 Days)

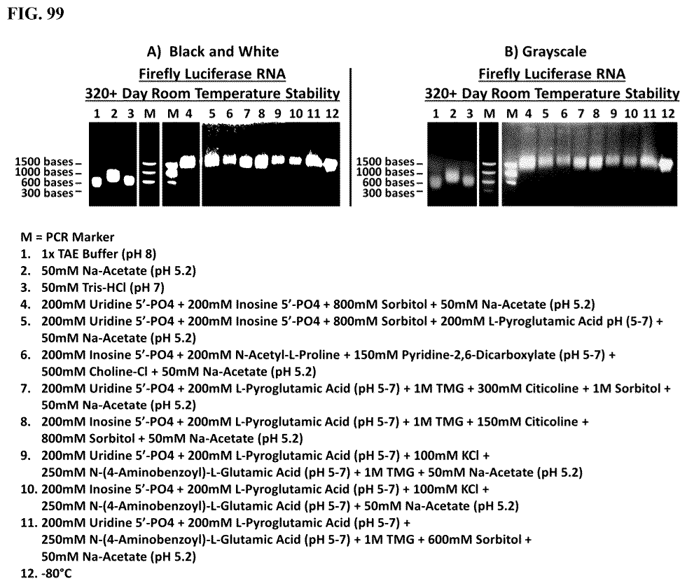

In vitro transcribed Firefly Luciferase RNA as described in Example 151-A above was diluted at a ratio of about 1:5 (about 400-1000 μg/mL) in different compositions containing 1×TAE Buffer (pH 8), 50 mM Na-Acetate (pH 5.2), or 50 mM Tris-HCl (pH 7) or various combinations of one or more RNA stabilizing substance with selected buffers as shown in FIGS. 99 A and B.
M=PCR Marker
1. 1×TAE Buffer (pH 8)
2. 50 mM Na-Acetate (pH 5.2)
3. 50 mM Tris-HCl (pH 7)
4. 200 mM Uridine 5′-PO4+200 mM Inosine 5′-PO4+800 mM Sorbitol+50 mM Na-Acetate (pH 5.2)
5. 200 mM Uridine 5′-PO4+200 mM Inosine 5′-PO4+800 mM Sorbitol+200 mM L-Pyroglutamic Acid pH (5-7)+50 mM Na-Acetate (pH 5.2)
6. 200 mM Inosine 5′-PO4+200 mM N-Acetyl-L-Proline+150 mM Pyridine-2,6-Dicarboxylate (pH 5-7)+500 mM Choline-Cl+50 mM Na-Acetate (pH 5.2)
7. 200 mM Uridine 5′-PO4+200 mM L-Pyroglutamic Acid (pH 5-7)+1M TMG+300 mM Citicoline+1M Sorbitol+50 mM Na-Acetate (pH 5.2)
8. 200 mM Inosine 5′-PO4+200 mM L-Pyroglutamic Acid (pH 5-7)+1M TMG+150 mM Citicoline+800 mM Sorbitol+50 mM Na-Acetate (pH 5.2)
9. 200 mM Uridine 5′-PO4+200 mM L-Pyroglutamic Acid (pH 5-7)+100 mM KCl+250 mM N-(4-Aminobenzoyl)-L-Glutamic Acid (pH 5-7)+1M TMG+50 mM Na-Acetate (pH 5.2)
10. 200 mM Inosine 5′-PO4+200 mM L-Pyroglutamic Acid (pH 5-7)+100 mM KCl+250 mM N-(4-Aminobenzoyl)-L-Glutamic Acid (pH 5-7)+50 mM Na-Acetate (pH 5.2)
11. 200 mM Uridine 5′-PO4+200 mM L-Pyroglutamic Acid (pH 5-7)+250 mM N-(4-Aminobenzoyl)-L-Glutamic Acid (pH 5-7)+1M TMG+600 mM Sorbitol+50 mM Na-Acetate (pH 5.2)
12. −80° C.

Following dilution of each sample in each respective RNA storage environment, samples were stored at room temperature (in a room with temperatures ranging from about 20° C. to 30° C.) over the course of about 320-360 days. Following storage for about 320-360 days, samples were then analyzed by denaturing agarose gel electrophoresis as shown in FIGS. 99A and B.

Prior to analysis by agarose gel electrophoresis, samples were diluted 2× with water and adjusted to pH 7 with 1M Tris-HCl (pH 7) to help improve analysis by gel electrophoresis.

1. A method of producing an RNA stabilizing composition comprising combining a stabilizing concentration of RNA stabilizing substance and an at least partially purified polymeric RNA substance that has undergone at least one purification step, wherein:
the RNA stabilizing substance comprises at least one of an exogenous mono-nucleotide substance of Formula 1, or conjugate acid, salt, or tautomer thereof: 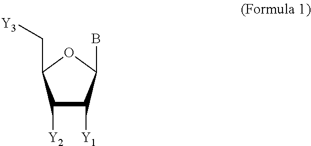
wherein:
Y₁, Y₂, and Y₃ are each selected from hydrogen (H), hydroxy (—OH), methoxy (—O—CH₃), ethoxy (—O—CH₂—CH₃), acetoxy (—O—(C═O)—CH₃), phosphate (—O—PO₃²⁻), diphosphate ((—O—PO₂⁻)—(O—PO₃²⁻)), and triphosphate ((—O—PO₂⁻)₂—(O—PO₃²⁻));
and at least one of Y₁, Y₂, or Y₃ is phosphate (—O—PO₃²⁻), diphosphate ((—O—PO₂⁻)—(O—PO₃²⁻)), or triphosphate ((—O—PO₂⁻)₂—(O—PO₃²⁻));
B is a nucleobase of the form A1 or A2: 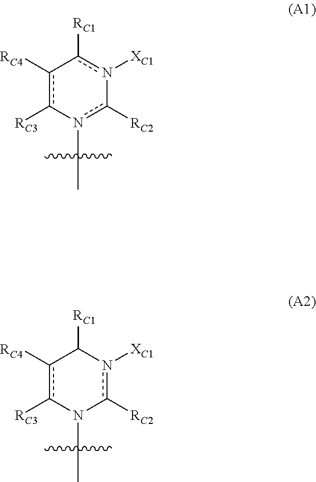
wherein: [symbol]
denotes a single or double bond; provided that if R_{C1} or R_{C2} is oxo, then the adjacent bond within the six membered ring is a single bond;
R_{C1} is selected from hydrogen (H), oxo (═O), amino (—NH₂), methylamino (—NH—CH₃), and dimethylamino (—N(CH₃)₂);
R_{C2} is selected from hydrogen (H), oxo (═O), amino (—NH₂), methylamino (—NH—CH₃), and dimethylamino (—N(CH₃)₂);
R_{C3} is selected from hydrogen (H) and carboxylate (—COO⁻);
R_{C4} is selected from hydrogen (H), methyl (—CH₃), hydroxymethyl (—CH₂—OH), and methoxy (—O—CH₃);
X_{C1} is absent or selected from hydrogen (H) and methyl (—CH₃); or
B is a nucleobase of the form: 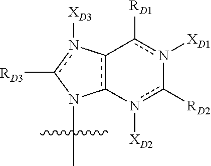
wherein: [symbol]
denotes a single or double bond; provided that if R_{P1}, R_{P2}, or R_{P3} is oxo, then the adjacent bond within the five or six membered ring is a single bond;
R_{P1} is selected from hydrogen (H), oxo (═O), amino (—NH₂), methylamino (—NH—CH₃), and dimethylamino (—N(CH₃)₂);
R_{P2} is selected from hydrogen (H), oxo (═O), amino (—NH₂), methylamino (—NH—CH₃), and dimethylamino (—N(CH₃)₂);
R_{P3} is selected from hydrogen (H), oxo (═O), and hydroxy (—OH);
X_{D1} is absent or selected from hydrogen (H) and methyl (—CH₃);
X_{D2} is absent or selected from hydrogen (H) and methyl (—CH₃);
X_{D3} is absent or selected from hydrogen (H) and methyl (—CH₃).

2. The method of claim 1, wherein the RNA stabilizing substance comprises at least one of an exogenous mono-nucleotide substance of Formula 1, wherein B is a nucleobase selected from uracil, thymine, cytosine, isocytosine, orotate, guanine, isoguanine, adenine, hypoxanthine, isohypoxanthine, xanthine, 2-pyrimidinone, and 4-pyrimidinone.

3. The method of claim 1, wherein the RNA stabilizing substance comprises at least one of an exogenous mono-nucleotide substance selected from orotidine-5′-monophosphate, 2′-deoxy-orotidine-5′-monophosphate, guanosine-5′-monophosphate, 2′-deoxyguanosine-5′-monophosphate, 8-oxo-guanosine-5′-monophosphate, 8-oxo-2′-deoxyguanosine-5′-monophosphate, isoguanosine-5′-monophosphate, 2′-deoxy-isoguanosine-5′-monophosphate, 8-oxo-isoguanosine-5′-monophosphate, 8-oxo-2′-deoxy-isoguanosine-5′-monophosphate, xanthosine-5′-monophosphate, 2 ‘-deoxyxanthosine-5’-monophosphate, 8-oxo-xanthosine-5′-monophosphate, 8-oxo-2′-deoxyxanthosine-5′-monophosphate, uridine-5′-monophosphate, 2′-deoxyuridine-5′-monophosphate, thymidine-5′-monophosphate, 5-methyl-uridine-5′-monophosphate, inosine-5′-monophosphate, 2′-deoxyinosine-5′-monophosphate, 8-oxo-inosine-5′-monophosphate, 8-oxo-2′-deoxyinosine-5′-monophosphate, cytidine-5′-monophosphate, 2′-deoxycytidine-5′-monophosphate, isoinosine-5′-monophosphate, 2′-deoxy-isoinosine-5′-monophosphate, 8-oxo-isoinosine-5′-monophosphate, 8-oxo-2′-deoxy-isoinosine-5′-monophosphate, isocytidine-5′-monophosphate, and 2′-deoxy-isocytidine-5′-monophosphate.

4. The method of claim 1, wherein the RNA stabilizing substance comprises at least one of an exogenous mono-nucleotide substance selected from orotidine-3′-monophosphate, 2′-deoxy-orotidine-3′-monophosphate, guanosine-3′-monophosphate, 2′-deoxyguanosine-3′-monophosphate, 8-oxo-guanosine-3′-monophosphate, 8-oxo-2′-deoxyguanosine-3′-monophosphate, isoguanosine-3′-monophosphate, 2′-deoxy-isoguanosine-3′-monophosphate, 8-oxo-isoguanosine-3′-monophosphate, 8-oxo-2′-deoxy-isoguanosine-3′-monophosphate, xanthosine-3′-monophosphate, 2 ‘-deoxyxanthosine-3’-monophosphate, 8-oxo-xanthosine-3′-monophosphate, 8-oxo-2′-deoxyxanthosine-3′-monophosphate, uridine-3′-monophosphate, 2′-deoxyuridine-3′-monophosphate, 5-methyl-uridine-3′-monophosphate, 5-methyl-2′-deoxyuridine-3′-monophosphate, inosine-3′-monophosphate, 2′-deoxyinosine-3′-monophosphate, 8-oxo-inosine-3′-monophosphate, 8-oxo-2′-deoxyinosine-3′-monophosphate, cytidine-3′-monophosphate, 2′-deoxycytidine-3′-monophosphate, isoinosine-3′-monophosphate, 2′-deoxy-isoinosine-3′-monophosphate, 8-oxo-isoinosine-3′-monophosphate, 8-oxo-2′-deoxy-isoinosine-3′-monophosphate, isocytidine-3′-monophosphate, and 2′-deoxy-isocytidine-3′-monophosphate.

5. The method of claim 1, wherein the RNA stabilizing substance is present in an amount of at least 25 micrograms.

6. The method of claim 1, further comprises combining a salt comprising an inorganic cation.

7. The method of claim 1, further comprises combining at least one cellular uptake agent comprising at least one of a lipid, a polymer, and a detergent.

8. The method of claim 1, wherein the at least partially purified polymeric RNA substance comprises at least one of a coding RNA and a non-coding RNA.

9. The method of claim 8 wherein the coding RNA comprises at least one of mRNA and self-amplifying RNA.

10. The method of claim 1, wherein the at least partially purified polymeric RNA substance is an active pharmaceutical ingredient in a pharmaceutical composition.

11. The method of claim 10, wherein the pharmaceutical composition is a medicament, a therapeutic agent, or a vaccine.

12. The method of claim 1, wherein the composition is contained in a hermetically sealed chamber.

13. The method of claim 12, wherein the hermetically sealed chamber is a syringe or a vial.

14. The method of claim 12, wherein the hermetically sealed chamber is stored at a temperature of at least 4° C. for a period of at least 7 days.

15. An RNA stabilizing composition comprising an at least partially purified polymeric RNA substance and a stabilizing concentration of RNA stabilizing substance,
wherein the RNA stabilizing substance comprises at least one of an exogenous mono-nucleotide substance of Formula 1, or conjugate acid, salt, or tautomer thereof: 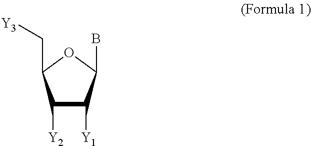
wherein:
Y₁, Y₂, and Y₃ are each selected from hydrogen (H), hydroxy (—OH), methoxy (—O—CH₃), ethoxy (—O—CH₂—CH₃), acetoxy (—O—(C═O)—CH₃), phosphate (—O—PO₃²⁻), and diphosphate ((—O—PO₂⁻)—(O—PO₃²⁻));
and at least one of Y₁, Y₂, or Y₃ is phosphate (—O—PO₃²⁻) or diphosphate ((—O—PO₂⁻)—(O—PO₃²⁻));
B is a nucleobase of the form A1 or A2: 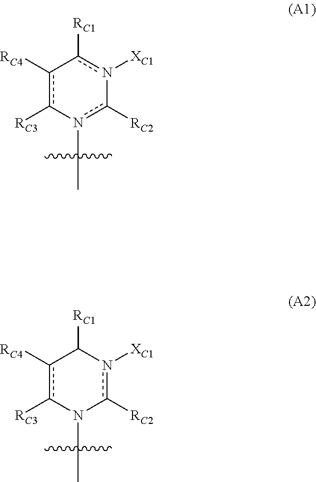
wherein: [symbol]
denotes a single or double bond; provided that if R_{C1} or R_{C2} is oxo, then the adjacent bond within the six membered ring is a single bond;
R_{C1} is selected from hydrogen (H), oxo (═O), amino (—NH₂), methylamino (—NH—CH₃), and dimethylamino (—N(CH₃)₂);
R_{C2} is selected from hydrogen (H), oxo (═O), amino (—NH₂), methylamino (—NH—CH₃), and dimethylamino (—N(CH₃)₂);
R_{C3} is selected from hydrogen (H) and carboxylate (—COO⁻);
R_{C4} is selected from hydrogen (H), methyl (—CH₃), hydroxymethyl (—CH₂—OH), and methoxy (—O—CH₃);
X_{C1} is absent or selected from hydrogen (H) and methyl (—CH₃); or
B is a nucleobase of the form: 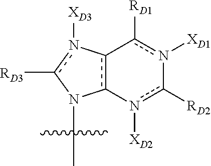
wherein: [symbol]
denotes a single or double bond; provided that if R_{P1}, R_{P2}, or R_{P3} is oxo, then the adjacent bond within the five or six membered ring is a single bond;
R_{P1} is selected from hydrogen (H), oxo (═O), amino (—NH₂), methylamino (—NH—CH₃), and dimethylamino (—N(CH₃)₂);
R_{P2} is selected from hydrogen (H), oxo (═O), amino (—NH₂), methylamino (—NH—CH₃), and dimethylamino (—N(CH₃)₂);
R_{P3} is selected from hydrogen (H), oxo (═O), and hydroxy (—OH);
X_{D1} is absent or selected from hydrogen (H) and methyl (—CH₃);
X_{D2} is absent or selected from hydrogen (H) and methyl (—CH₃);
X_{D3} is absent or selected from hydrogen (H) and methyl (—CH₃).

16. The composition of claim 15, wherein the RNA stabilizing substance comprises at least one of an exogenous mono-nucleotide substance of Formula 1, wherein B is a nucleobase selected from uracil, thymine, cytosine, isocytosine, orotate, guanine, isoguanine, adenine, hypoxanthine, isohypoxanthine, xanthine, 2-pyrimidinone, and 4-pyrimidinone.

17. The composition of claim 15, wherein the RNA stabilizing substance comprises at least one of an exogenous mono-nucleotide substance selected from orotidine-5′-monophosphate, 2′-deoxy-orotidine-5′-monophosphate, guanosine-5′-monophosphate, 2′-deoxyguanosine-5′-monophosphate, 8-oxo-guanosine-5′-monophosphate, 8-oxo-2′-deoxyguanosine-5′-monophosphate, isoguanosine-5′-monophosphate, 2′-deoxy-isoguanosine-5′-monophosphate, 8-oxo-isoguanosine-5′-monophosphate, 8-oxo-2′-deoxy-isoguanosine-5′-monophosphate, xanthosine-5′-monophosphate, 2′-deoxyxanthosine-5′-monophosphate, 8-oxo-xanthosine-5′-monophosphate, 8-oxo-2′-deoxyxanthosine-5′-monophosphate, uridine-5′-monophosphate, 2′-deoxyuridine-5′-monophosphate, thymidine-5′-monophosphate, 5-methyl-uridine-5′-monophosphate, inosine-5′-monophosphate, 2′-deoxyinosine-5′-monophosphate, 8-oxo-inosine-5′-monophosphate, 8-oxo-2′-deoxyinosine-5′-monophosphate, cytidine-5′-monophosphate, 2′-deoxycytidine-5′-monophosphate, isoinosine-5′-monophosphate, 2′-deoxy-isoinosine-5′-monophosphate, 8-oxo-isoinosine-5′-monophosphate, 8-oxo-2′-deoxy-isoinosine-5′-monophosphate, isocytidine-5′-monophosphate, and 2′-deoxy-isocytidine-5′-monophosphate.

18. The composition of claim 15, wherein the RNA stabilizing substance comprises at least one of an exogenous mono-nucleotide substance selected from orotidine-3′-monophosphate, 2′-deoxy-orotidine-3′-monophosphate, guanosine-3′-monophosphate, 2′-deoxyguanosine-3′-monophosphate, 8-oxo-guanosine-3′-monophosphate, 8-oxo-2′-deoxyguanosine-3′-monophosphate, isoguanosine-3′-monophosphate, 2′-deoxy-isoguanosine-3′-monophosphate, 8-oxo-isoguanosine-3′-monophosphate, 8-oxo-2′-deoxy-isoguanosine-3′-monophosphate, xanthosine-3′-monophosphate, 2′-deoxyxanthosine-3′-monophosphate, 8-oxo-xanthosine-3′-monophosphate, 8-oxo-2′-deoxyxanthosine-3′-monophosphate, uridine-3′-monophosphate, 2′-deoxyuridine-3′-monophosphate, 5-methyl-uridine-3′-monophosphate, 5-methyl-2′-deoxyuridine-3′-monophosphate, inosine-3′-monophosphate, 2′-deoxyinosine-3′-monophosphate, 8-oxo-inosine-3′-monophosphate, 8-oxo-2′-deoxyinosine-3′-monophosphate, cytidine-3′-monophosphate, 2′-deoxycytidine-3′-monophosphate, isoinosine-3′-monophosphate, 2′-deoxy-isoinosine-3′-monophosphate, 8-oxo-isoinosine-3′-monophosphate, 8-oxo-2′-deoxy-isoinosine-3′-monophosphate, isocytidine-3′-monophosphate, and 2′-deoxy-isocytidine-3′-monophosphate.

19. The composition of claim 15, wherein the RNA stabilizing substance comprises total mono-nucleotide weight in an amount of at least 1 mg.

20. The composition of claim 15, wherein the mono-nucleotides comprise at least 0.1% of the total composition by weight.

21. The composition of claim 15, further comprises at least one of an amino acid and an inorganic cation.

22. The composition of claim 15, further comprises at least one cellular uptake agent comprising at least one of a cationic lipid, an ionizable lipid, a polymer-conjugated lipid, and a neutral lipid.

23. The composition of claim 22, wherein the ratio of the at least partially purified polymeric RNA substance to the cellular uptake agent lipids in the composition is at least 1:10 on a weight-by-weight basis.

24. The composition of claim 15, wherein the at least partially purified polymeric RNA substance comprises at least one of a coding RNA and a non-coding RNA.

25. The composition of claim 24, wherein the coding RNA comprises at least one of mRNA and self-amplifying RNA.

26. The composition of claim 15, wherein the at least partially purified polymeric RNA substance is an active pharmaceutical ingredient in a pharmaceutical composition.

27. The composition of claim 26, wherein the pharmaceutical composition is a medicament, a therapeutic agent, or a vaccine.

28. The composition of claim 15, wherein the viscosity of the composition is at least 100 centipoise at 20° C.

29. A method of providing a pharmaceutical composition in a chamber comprising combining to said chamber a stabilizing concentration of RNA stabilizing substance, an RNA substance that is an active pharmaceutical ingredient, and at least one cellular uptake agent, wherein the RNA stabilizing substance comprises at least one of an exogenous mono-nucleotide substance of Formula 1, or conjugate acid, salt, or tautomer thereof: 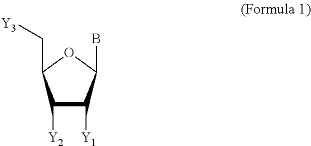
wherein:
Y₁, Y₂, and Y₃ are each selected from hydrogen (H), hydroxy (—OH), methoxy (—O—CH₃), ethoxy (—O—CH₂—CH₃), acetoxy (—O—(C═O)—CH₃), phosphate (—O—PO₃²⁻), diphosphate ((—O—PO₂⁻)—(O—PO₃²⁻)), and triphosphate ((—O—PO₂⁻)₂—(O—PO₃²⁻));
and at least one of Y₁, Y₂, or Y₃ is phosphate (—O—PO₃²⁻), diphosphate ((—O—PO₂⁻)—(O—PO₃²⁻)), or triphosphate ((—O—PO₂⁻)₂—(O—PO₃²⁻));
B is a nucleobase selected from a pyrimidine nucleobase or a purine nucleobase.

30. The method of claim 29, wherein the RNA stabilizing substance comprises at least one of an exogenous mono-nucleotide substance of Formula 1, wherein B is a purine nucleobase selected from guanine, isoguanine, adenine, hypoxanthine, isohypoxanthine, and xanthine.

31. The method of claim 29, wherein the RNA stabilizing substance comprises at least one of an exogenous mono-nucleotide substance of Formula 1, wherein B is a pyrimidine nucleobase selected from uracil, thymine, cytosine, isocytosine, orotate, 2-pyrimidinone, and 4-pyrimidinone.

32. The method of claim 29, wherein the RNA stabilizing substance comprises at least one of an exogenous mono-nucleotide substance selected from orotidine-5′-monophosphate, 2′-deoxy-orotidine-5′-monophosphate, guanosine-5′-monophosphate, 2′-deoxyguanosine-5′-monophosphate, 8-oxo-guanosine-5′-monophosphate, 8-oxo-2′-deoxyguanosine-5′-monophosphate, isoguanosine-5′-monophosphate, 2′-deoxy-isoguanosine-5′-monophosphate, 8-oxo-isoguanosine-5′-monophosphate, 8-oxo-2′-deoxy-isoguanosine-5′-monophosphate, xanthosine-5′-monophosphate, 2′-deoxyxanthosine-5′-monophosphate, 8-oxo-xanthosine-5′-monophosphate, 8-oxo-2′-deoxyxanthosine-5′-monophosphate, uridine-5′-monophosphate, 2′-deoxyuridine-5′-monophosphate, thymidine-5′-monophosphate, 5-methyl-uridine-5′-monophosphate, inosine-5′-monophosphate, 2′-deoxyinosine-5′-monophosphate, 8-oxo-inosine-5′-monophosphate, 8-oxo-2′-deoxyinosine-5′-monophosphate, cytidine-5′-monophosphate, 2′-deoxycytidine-5′-monophosphate, isoinosine-5′-monophosphate, 2′-deoxy-isoinosine-5′-monophosphate, 8-oxo-isoinosine-5′-monophosphate, 8-oxo-2′-deoxy-isoinosine-5′-monophosphate, isocytidine-5′-monophosphate, and 2′-deoxy-isocytidine-5′-monophosphate.

33. The method of claim 29, wherein the RNA stabilizing substance comprises at least one of an exogenous mono-nucleotide substance selected from orotidine-3′-monophosphate, 2′-deoxy-orotidine-3′-monophosphate, guanosine-3′-monophosphate, 2′-deoxyguanosine-3′-monophosphate, 8-oxo-guanosine-3′-monophosphate, 8-oxo-2′-deoxyguanosine-3′-monophosphate, isoguanosine-3′-monophosphate, 2′-deoxy-isoguanosine-3′-monophosphate, 8-oxo-isoguanosine-3′-monophosphate, 8-oxo-2′-deoxy-isoguanosine-3′-monophosphate, xanthosine-3′-monophosphate, 2′-deoxyxanthosine-3′-monophosphate, 8-oxo-xanthosine-3′-monophosphate, 8-oxo-2′-deoxyxanthosine-3′-monophosphate, uridine-3′-monophosphate, 2′-deoxyuridine-3′-monophosphate, 5-methyl-uridine-3′-monophosphate, 5-methyl-2′-deoxyuridine-3′-monophosphate, inosine-3′-monophosphate, 2′-deoxyinosine-3′-monophosphate, 8-oxo-inosine-3′-monophosphate, 8-oxo-2′-deoxyinosine-3′-monophosphate, cytidine-3′-monophosphate, 2′-deoxycytidine-3′-monophosphate, isoinosine-3′-monophosphate, 2′-deoxy-isoinosine-3′-monophosphate, 8-oxo-isoinosine-3′-monophosphate, 8-oxo-2′-deoxy-isoinosine-3′-monophosphate, isocytidine-3′-monophosphate, and 2′-deoxy-isocytidine-3′-monophosphate.

34. The method of claim 29, wherein the chamber is stored at a minimum temperature of at least 4° C. for at least 1 week.

35. The method of claim 29, wherein the chamber is at least one of packaged, transported, or shipped.

36. The method of claim 35, wherein the viscosity of the composition in the chamber is at least 100 centipoise at 20° C. when the chamber is at least one of packaged, transported, or shipped.

37. The method of claim 29, wherein the chamber is hermetically sealed.

38. The method of claim 37, wherein the hermetically sealed chamber is a syringe or a vial.

39. The method of claim 29, wherein the pharmaceutical composition is administered in an effective amount to a subject in need thereof.

40. The method of claim 29, wherein the pharmaceutical composition is a medicament, a therapeutic agent, or a vaccine.

41. The method of claim 29, wherein the at least one cellular uptake agent comprises at least one of lipid and polymer.

42-51. (canceled)

52. The method of claim 41, wherein the lipid comprises at least one of a cationic lipid, an ionizable lipid, a polymer-conjugated lipid, and a neutral lipid.

53. The method of claim 41, wherein the polymer comprises at least one of a cationic polymer, an ionizable polymer, and a zwitterionic polymer.
